(19) United States
(12) Patent Application Publication
(10) Pub. No.: US 2024/0293526 A1
(43) Pub. Date: Sep. 5, 2024

(54) EBOLAVIRUS AND MARBURGVIRUS VACCINES

(71) Applicant: CureVac SE, Tübingen (DE)

(72) Inventors: Susanne RAUCH, Tübingen (DE); Edith JASNY, Tübingen (DE)

(73) Assignee: CureVac SE, Tübingen (DE)

(21) Appl. No.: 18/427,737

(22) Filed: Jan. 30, 2024

Related U.S. Application Data
(63) Continuation of application No. 15/536,595, filed on Jun. 15, 2017, now abandoned.

(30) Foreign Application Priority Data
Dec. 16, 2014 (WO) PCT/EP2014/003371

(51) Int. Cl. A61K 39/12; A61K 39/00; C07K 14/005; C12N 7/00

(52) U.S. Cl. CPC A61K 39/12; C07K 14/005; C12N 7/00; A61K 2039/53; A61K 2039/54; A61K 2039/575; A61K 2039/70; C12N 2760/14134; C12N 2760/14234; C12N 2830/50

(57) ABSTRACT

The present invention relates to an mRNA sequence, comprising a coding region, encoding at least one antigenic peptide or protein derived from the glycoprotein (GP) and/or the matrix protein 40 (VP40) and/or the nucleoprotein (NP) of a virus of the genus Ebolavirus or Marburgvirus or a fragment, variant or derivative thereof. Additionally, the present invention relates to a composition comprising a plurality of mRNA sequences comprising a coding region, encoding at least one antigenic peptide or protein derived from the glycoprotein (GP) and/or the matrix protein 40 (VP40) and/or the nucleoprotein (NP) of a virus of the genus Ebolavirus or Marburgvirus or a fragment, variant or derivative thereof. Furthermore it also discloses the use of the mRNA sequence or the composition comprising a plurality of mRNA sequences for the preparation of a pharmaceutical composition, especially a vaccine, e.g. for use in the prophylaxis or treatment of Ebolavirus or Marburgvirus infections. The present invention further describes a method of treatment or prophylaxis of Ebolavirus or Marburgvirus infections using the mRNA sequence.

This application is a continuation of U.S. application Ser. No. 15/536,595, filed Jun. 15, 2017, which is a national phase application under 35 U.S.C. § 371 of International Application No. PCT/EP2015/080108, filed Dec. 16, 2015, which claims the benefit of international patent application PCT/EP2014/003371, filed Dec. 16, 2014, each of which is incorporated herein by reference in its entirety.

This application contains a Sequence Listing XML, which has been submitted electronically and is hereby incorporated by reference in its entirety. Said Sequence Listing XML, created on Jan. 26, 2024, is named CRVCP0170USC1.xml and is 671,392 bytes in size.

The present invention relates to mRNA sequences usable as RNA-based vaccines against infections with Ebolaviruses and Marburgviruses. Additionally, the present invention relates to a composition comprising a plurality of mRNA sequences and the use of the mRNA sequence or the composition for the preparation of a pharmaceutical composition, especially a vaccine, e.g. for use in the prophylaxis, postexposure prophylaxis or treatment of Ebolavirus or Marburgvirus infections. The present invention further describes a method of treatment, postexposure prophylaxis or prophylaxis of infections with Ebolavirus or Marburgvirus using the mRNA sequence.

Ebolaviruses and the genetically-related Marburgviruses are human pathogens that cause severe diseases. Ebolaviruses and Marburgviruses are filoviruses, which are enveloped viruses featuring a negative-stranded RNA genome. The family of Filoviridae comprises three genera: Ebolavirus, Marburgvirus and Cuevavirus. The genus of Cuevaviruses as well as Marburgviruses include only one species, i.e. Lloviu cuevavirus (Lloviu virus—LLOV) and Marburg marburgvirus, respectively, which is subdivided in Marburg virus (MARV) and Ravn virus (RAVV). The genus of Ebolaviruses comprises five known species, i.e. Bundibugyo ebolavirus (Bundibugyo virus—BDBV), Reston ebolavirus (Reston virus—RESTV), Sudan ebolavirus (Sudan virus—SUDV), Taï Forest ebolavirus (Taï Forest virus—TAFV) (=Côte d'Ivoire ebolavirus), and Zaire ebolavirus (Ebola virus—EBOV). While Cuevaviruses have been isolated from bats and their potential as a pathogen in humans remains unknown, both Ebolaviruses and Marburgviruses are human pathogens that cause Ebolavirus disease (EVD) and Marburgvirus disease, respectively, characterised by haemorrhagic fever and an extremely high mortality rate. Both virus genera have been the cause of large outbreaks: two outbreaks of Marburgvirus with >100 deaths and death rates >80% have been recorded so far in the Congo and Angola, respectively. Ebolaviruses have been the cause of regular outbreaks every 10-15 years with EBOV, SUDV and BDBV as the causative viral species. Outbreaks have greatly varied in size with the last large outbreak reported in 2000-2001 in Uganda with 425 cases (Okware S. I. et al. (2002), Tropical Medicine and International Health, vol. 7, no. 12, 1068-1075). The 2014 Ebolavirus epidemic is by far the largest in history, affecting multiple countries in West Africa with case reports in Europe and the USA. The WHO situation report from Dec. 11, 2014 specifies a total of 14,098 infected patients with 5160 reported deaths in 6 countries (Guinea, Liberia, Mali, Sierra Leone, Spain and the USA). Despite the unprecedented proportions of the 2014 outbreak, the epidemic features a comparable course of infection, incubation period and serial interval to previous outbreaks (WHO Ebola Response Team (2014), N ENGL J MED, vol. 371, no. 16, 1481-1495) indicative of factors other than the virus itself causing the large number of infections.

Counter measures at present include the isolation of patients, identification and isolation of contacts and ensuring safety measures during burials (Borchert M. et al. (2011), BMC Infec. Dis., vol. 11, 357), which can help to limit an EBOV outbreak (Okware S. I. et al. (2002), Tropical Medicine and International Health, vol. 7, no. 12, 1068-1075) but have been inefficient in the 2014 epidemic. Importantly, current treatment of infected patients is restricted to palliative care and no prophylactic and therapeutic treatments are licenced at present. The dramatic situation of the current outbreak and the high risk of future Ebolavirus and Marburgvirus outbreaks demonstrate that the development of an effective and prophylactic treatment is of paramount importance.

A multitude of classical, subunit, and virus-vectored approaches have been attempted for development of a vaccine to protect against lethal Ebola virus (EBOV) and Marburg virus (MARV) infections. Classic methods for vaccine development, including producing and testing attenuated and inactivated viral preparations, have been tried with moderate success; however, the risk of revertants or incomplete inactivation are unacceptable for future use of such vaccines in humans. Additionally, multiple vector-based approaches including replication-incompetent Venezuelan equine encephalitis virus replicons, replication-incompetent adenoviral vectors, vaccinia- and parainfluenza-vectored vaccines, and live, recombinant virus-based approaches using vesicular stomatitis virus (VSV) for vaccination have been explored, leading to nearly complete or complete protection against filovirus hemorrhagic fever in non-human primates. As an alternative to vector-based vaccine platforms, virus-like particles (VLPs) are emerging as promising vaccine candidates for filovirus hemorrhagic fever. The technology is based on assembly of filovirus GP, the main protective antigen, with matrix protein (VP40) into VLPs after coexpression in eukaryotic cells (reviewed in Warfield K. L. and Aman M. J. (2011), JID, 204 (Suppl 3)). Furthermore, the international patent application WO 99/32147 describes Ebolavirus DNA-based vaccines. The nucleic acid molecule encodes the transmembrane form of the viral glycoprotein (GP) or the secreted form of the viral glycoprotein (sGP) or the viral nucleoprotein (NP).

Nevertheless there is still a need for an effective and safe Ebolavirus and Marburgvirus vaccine. The vaccine should be deliverable at any time, therefore a very quick production should be possible. Furthermore due to the geographical distribution of Ebola- and Marburgvirus outbreaks, there is an urgent need for a temperature stable Ebolavirus and Marburgvirus vaccine which is not dependent on cooling (cold chain).

Furthermore, there is an unmet medical need to improve the effectiveness of Ebolavirus and Marburgvirus vaccine delivery and for the development of a safe and effective Ebolavirus and Marburgvirus vaccine that is affordable and can be manufactured rapidly.

Therefore, it is the object of the underlying invention to provide nucleotide sequences coding for antigenic peptides or proteins of a virus of the genus Ebolavirus or Marburgvirus for the use as a vaccine for prophylaxis or treatment of Ebolavirus or Marburgvirus infections, particularly for preexposure prophylaxis or postexposure prophylaxis. Furthermore, it is the object of the present invention to provide an effective Ebolavirus or Marburgvirus vaccine which can be stored without cold chain and which enables rapid and scalable vaccine production.

These objects are solved by the subject matter of the attached claims. Particularly, the objects underlying the present invention are solved according to a first aspect by an inventive mRNA sequence comprising a coding region, encoding at least one antigenic peptide or protein of a virus of the genus Ebolavirus or Marburgvirus or a fragment, variant or derivative thereof.

For the sake of clarity and readability, the following scientific background information and definitions are provided. Any technical features disclosed thereby can be part of each and every embodiment of the invention. Additional definitions and explanations can be provided in the context of this disclosure.

Immune system: The immune system may protect organisms from infection. If a pathogen breaks through a physical barrier of an organism and enters this organism, the innate immune system provides an immediate, but non-specific response. If pathogens evade this innate response, vertebrates possess a second layer of protection, the adaptive immune system. Here, the immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts contains so called humoral and cellular components.

Immune response: An immune response may typically either be a specific reaction of the adaptive immune system to a particular antigen (so called specific or adaptive immune response) or an unspecific reaction of the innate immune system (so called unspecific or innate immune response). The invention relates to the core to specific reactions (adaptive immune responses) of the adaptive immune system. Particularly, it relates to adaptive immune responses to infections by viruses like e.g. Ebolavirus or Marburgvirus. However, this specific response can be supported by an additional unspecific reaction (innate immune response). Therefore, the invention also relates to a compound for simultaneous stimulation of the innate and the adaptive immune system to evoke an efficient adaptive immune response.

Adaptive immune system: The adaptive immune system is composed of highly specialized, systemic cells and processes that eliminate or prevent pathogenic growth. The adaptive immune response provides the vertebrate immune system with the ability to recognize and remember specific pathogens (to generate immunity), and to mount stronger attacks each time the pathogen is encountered. The system is highly adaptable because of somatic hypermutation (a process of increased frequency of somatic mutations), and V(D)J recombination (an irreversible genetic recombination of antigen receptor gene segments). This mechanism allows a small number of genes to generate a vast number of different antigen receptors, which are then uniquely expressed on each individual lymphocyte. Because the gene rearrangement leads to an irreversible change in the DNA of each cell, all of the progeny (offspring) of that cell will then inherit genes encoding the same receptor specificity, including the Memory B cells and Memory T cells that are the keys to long-lived specific immunity. Immune network theory is a theory of how the adaptive immune system works, that is based on interactions between the variable regions of the receptors of T cells, B cells and of molecules made by T cells and B cells that have variable regions.

Adaptive immune response: The adaptive immune response is typically understood to be antigen-specific. Antigen specificity allows for the generation of responses that are tailored to specific antigens, pathogens or pathogen-infected cells. The ability to mount these tailored responses is maintained in the body by “memory cells”. Should a pathogen infect the body more than once, these specific memory cells are used to quickly eliminate it. In this context, the first step of an adaptive immune response is the activation of naïve antigen-specific T cells or different immune cells able to induce an antigen-specific immune response by antigen-presenting cells. This occurs in the lymphoid tissues and organs through which naïve T cells are constantly passing. Cell types that can serve as antigen-presenting cells are inter alia dendritic cells, macrophages, and B cells. Each of these cells has a distinct function in eliciting immune responses. Dendritic cells take up antigens by phagocytosis and macropinocytosis and are stimulated by contact with e.g. a foreign antigen to migrate to the local lymphoid tissue, where they differentiate into mature dendritic cells. Macrophages ingest particulate antigens such as bacteria and are induced by infectious agents or other appropriate stimuli to express MHC molecules. The unique ability of B cells to bind and internalize soluble protein antigens via their receptors may also be important to induce T cells. Presenting the antigen on MHC molecules leads to activation of T cells which induces their proliferation and differentiation into armed effector T cells. The most important function of effector T cells is the killing of infected cells by CD8+ cytotoxic T cells and the activation of macrophages by Th1 cells which together make up cell-mediated immunity, and the activation of B cells by both Th2 and Th1 cells to produce different classes of antibody, thus driving the humoral immune response. T cells recognize an antigen by their T cell receptors which do not recognize and bind antigen directly, but instead recognize short peptide fragments e.g. of pathogen-derived protein antigens, which are bound to MHC molecules on the surfaces of other cells.

Cellular immunity/cellular immune response: Cellular immunity relates typically to the activation of macrophages, natural killer cells (NK), antigen-specific cytotoxic T-lymphocytes, and the release of various cytokines in response to an antigen. In a more general way, cellular immunity is not related to antibodies but to the activation of cells of the immune system. A cellular immune response is characterized e.g. by activating antigen-specific cytotoxic T-lymphocytes that are able to induce apoptosis in body cells displaying epitopes of an antigen on their surface, such as virus-infected cells, cells with intracellular bacteria, and cancer cells displaying tumor antigens; activating macrophages and natural killer cells, enabling them to destroy pathogens; and stimulating cells to secrete a variety of cytokines that influence the function of other cells involved in adaptive immune responses and innate immune responses.

Humoral immunity/humoral immune response: Humoral immunity refers typically to antibody production and the accessory processes that may accompany it. A humoral immune response may be typically characterized, e.g., by Th2 activation and cytokine production, germinal center formation and isotype switching, affinity maturation and memory cell generation. Humoral immunity also typically may refer to the effector functions of antibodies, which include pathogen and toxin neutralization, classical complement activation, and opsonin promotion of phagocytosis and pathogen elimination.

Innate immune system: The innate immune system, also known as non-specific immune system, comprises the cells and mechanisms that defend the host from infection by other organisms in a non-specific manner. This means that the cells of the innate system recognize and respond to pathogens in a generic way, but unlike the adaptive immune system, it does not confer long-lasting or protective immunity to the host. The innate immune system may be e.g. activated by ligands of pathogen-associated molecular patterns (PAMP) receptors, e.g. Toll-like receptors (TLRs) or other auxiliary substances such as lipopolysaccharides, TNF-alpha, CD40 ligand, or cytokines, monokines, lymphokines, interleukins or chemokines, IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta, TNF-alpha, growth factors, and hGH, a ligand of human Toll-like receptor TLR1, TLR2, TLR3, TLR4, TLR5, TLR6, TLR7, TLR8, TLR9, TLR10, a ligand of murine Toll-like receptor TLR1, TLR2, TLR3, TLR4, TLR5, TLR6, TLR7, TLR8, TLR9, TLR10, TLR11, TLR12 or TLR13, a ligand of a NOD-like receptor, a ligand of a RIG-I like receptor, an immunostimulatory nucleic acid, an immunostimulatory RNA (isRNA), a CpG-DNA, an antibacterial agent, or an anti-viral agent. Typically a response of the innate immune system includes recruiting immune cells to sites of infection, through the production of chemical factors, including specialized chemical mediators, called cytokines; activation of the complement cascade; identification and removal of foreign substances present in organs, tissues, the blood and lymph, by specialized white blood cells; activation of the adaptive immune system through a process known as antigen presentation; and/or acting as a physical and chemical barrier to infectious agents.

Adjuvant/adjuvant component: An adjuvant or an adjuvant component in the broadest sense is typically a (e.g. pharmacological or immunological) agent or composition that may modify, e.g. enhance, the efficacy of other agents, such as a drug or vaccine. Conventionally the term refers in the context of the invention to a compound or composition that serves as a carrier or auxiliary substance for immunogens and/or other pharmaceutically active compounds. It is to be interpreted in a broad sense and refers to a broad spectrum of substances that are able to increase the immunogenicity of antigens incorporated into or co-administered with an adjuvant in question. In the context of the present invention an adjuvant will preferably enhance the specific immunogenic effect of the active agents of the present invention. Typically, “adjuvant” or “adjuvant component” has the same meaning and can be used mutually. Adjuvants may be divided, e.g., into immuno potentiators, antigenic delivery systems or even combinations thereof.

The term “adjuvant” is typically understood not to comprise agents which confer immunity by themselves. An adjuvant assists the immune system unspecifically to enhance the antigen-specific immune response by e.g. promoting presentation of an antigen to the immune system or induction of an unspecific innate immune response. Furthermore, an adjuvant may preferably e.g. modulate the antigen-specific immune response by e.g. shifting the dominating Th2-based antigen specific response to a more Th1-based antigen specific response or vice versa. Accordingly, an adjuvant may favourably modulate cytokine expression/secretion, antigen presentation, type of immune response etc.

Immunostimulatory RNA: An immunostimulatory RNA (isRNA) in the context of the invention may typically be an RNA that is able to induce an innate immune response itself. It usually does not have an open reading frame and thus does not provide a peptide-antigen or immunogen but elicits an innate immune response e.g. by binding to a specific kind of Toll-like-receptor (TLR) or other suitable receptors. However, of course also mRNAs having an open reading frame and coding for a peptide/protein (e.g. an antigenic function) may induce an innate immune response.

Antigen: According to the present invention, the term “antigen” refers typically to a substance which may be recognized by the immune system and may be capable of triggering an antigen-specific immune response, e.g. by formation of antibodies or antigen-specific T-cells as part of an adaptive immune response. An antigen may be a protein or peptide. In this context, the first step of an adaptive immune response is the activation of naïve antigen-specific T cells by antigen-presenting cells. This occurs in the lymphoid tissues and organs through which naïve T cells are constantly passing. The three cell types that can serve as antigen-presenting cells are dendritic cells, macrophages, and B cells. Each of these cells has a distinct function in eliciting immune responses. Tissue dendritic cells take up antigens by phagocytosis and macropinocytosis and are stimulated by infection to migrate to the local lymphoid tissue, where they differentiate into mature dendritic cells. Macrophages ingest particulate antigens such as bacteria and are induced by infectious agents to express MHC class II molecules. The unique ability of B cells to bind and internalize soluble protein antigens via their receptors may be important to induce T cells. By presenting the antigen on MHC molecules leads to activation of T cells which induces their proliferation and differentiation into armed effector T cells. The most important function of effector T cells is the killing of infected cells by CD8+ cytotoxic T cells and the activation of macrophages by Th1 cells which together make up cell-mediated immunity, and the activation of B cells by both Th2 and Th1 cells to produce different classes of antibody, thus driving the humoral immune response. T cells recognize an antigen by their T cell receptors which does not recognize and bind antigen directly, but instead recognize short peptide fragments e.g. of pathogens' protein antigens, which are bound to MHC molecules on the surfaces of other cells.

T cells fall into two major classes that have different effector functions. The two classes are distinguished by the expression of the cell-surface proteins CD4 and CD8. These two types of T cells differ in the class of MHC molecule that they recognize. There are two classes of MHC molecules—MHC class I and MHC class II molecules—which differ in their structure and expression pattern on tissues of the body. CD4+ T cells bind to a MHC class II molecule and CD8+ T cells to a MHC class I molecule. MHC class I and MHC class II molecules have distinct distributions among cells that reflect the different effector functions of the T cells that recognize them. MHC class I molecules present peptides of cytosolic and nuclear origin e.g. from pathogens, commonly viruses, to CD8+ T cells, which differentiate into cytotoxic T cells that are specialized to kill any cell that they specifically recognize. Almost all cells express MHC class I molecules, although the level of constitutive expression varies from one cell type to the next. But not only pathogenic peptides from viruses are presented by MHC class I molecules, also self-antigens like tumour antigens are presented by them. MHC class I molecules bind peptides from proteins degraded in the cytosol and transported in the endoplasmic reticulum. The CD8+ T cells that recognize MHC class I:peptide complexes at the surface of infected cells are specialized to kill any cells displaying foreign peptides and so rid the body of cells infected with viruses and other cytosolic pathogens.

The main function of CD4+ T cells (CD4+ helper T cells) that recognize MHC class II molecules is to activate other effector cells of the immune system. Thus MHC class II molecules are normally found on B lymphocytes, dendritic cells, and macrophages, cells that participate in immune responses, but not on other tissue cells. Macrophages, for example, are activated to kill the intravesicular pathogens they harbour, and B cells to secrete immunoglobulins against foreign molecules. MHC class II molecules are prevented from binding to peptides in the endoplasmic reticulum and thus MHC class II molecules bind peptides from proteins which are degraded in endosomes. They can capture peptides from pathogens that have entered the vesicular system of macrophages, or from antigens internalized by immature dendritic cells or the immunoglobulin receptors of B cells. Pathogens that accumulate in large numbers inside macrophage and dendritic cell vesicles tend to stimulate the differentiation of Th1 cells, whereas extracellular antigens tend to stimulate the production of Th2 cells. Th1 cells activate the microbicidal properties of macrophages and induce B cells to make IgG antibodies that are very effective of opsonising extracellular pathogens for ingestion by phagocytic cells, whereas Th2 cells initiate the humoral response by activating naïve B cells to secrete IgM, and induce the production of weakly opsonising antibodies such as IgG1 and IgG3 (mouse) and IgG2 and IgG4 (human) as well as IgA and IgE (mouse and human).

Epitope (also called “antigen determinant”): T cell epitopes or parts of the proteins in the context of the present invention may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T cells in form of a complex consisting of the peptide fragment and an MHC molecule.

B cell epitopes are typically fragments located on the outer surface of (native) protein or peptide antigens as defined herein, preferably having 5 to 15 amino acids, more preferably having 5 to 12 amino acids, even more preferably having 6 to 9 amino acids, which may be recognized by antibodies, i.e. in their native form.

Such epitopes of proteins or peptides may furthermore be selected from any of the herein mentioned variants of such proteins or peptides. In this context antigenic determinants can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain.

Vaccine: A vaccine is typically understood to be a prophylactic or therapeutic material providing at least one antigen or antigenic function. The antigen or antigenic function may stimulate the body's adaptive immune system to provide an adaptive immune response.

Antigen-providing mRNA: An antigen-providing mRNA in the context of the invention may typically be an mRNA, having at least one open reading frame that can be translated by a cell or an organism provided with that mRNA. The product of this translation is a peptide or protein that may act as an antigen, preferably as an immunogen. The product may also be a fusion protein composed of more than one immunogen, e.g. a fusion protein that consist of two or more epitopes, peptides or proteins derived from the same or different virus-proteins, wherein the epitopes, peptides or proteins may be linked by linker sequences.

Bi-/multicistronic mRNA: mRNA, that typically may have two (bicistronic) or more (multicistronic) open reading frames (ORF). An open reading frame in this context is a sequence of several nucleotide triplets (codons) that can be translated into a peptide or protein. Translation of such an mRNA yields two (bicistronic) or more (multicistronic) distinct translation products (provided the ORFs are not identical). For expression in eukaryotes such mRNAs may for example comprise an internal ribosomal entry site (IRES) sequence.

5′-CAP-Structure: A 5′-CAP is typically a modified nucleotide, particularly a guanine nucleotide, added to the 5′ end of an mRNA-molecule. Preferably, the 5′-CAP is added using a 5′-5′-triphosphate linkage (also named m7GpppN). Further examples of 5′-CAP structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. These modified 5′-CAP structures may be used in the context of the present invention to modify the inventive mRNA sequence. Further modified 5′-CAP structures which may be used in the context of the present invention are CAP1 (methylation of the ribose of the adjacent nucleotide of m7GpppN), CAP2 (methylation of the ribose of the 2^{nd} nucleotide downstream of the m7GpppN), CAP3 (methylation of the ribose of the 3^{rd} nucleotide downstream of the m7GpppN), CAP4 (methylation of the ribose of the 4^{th} nucleotide downstream of the m7GpppN), ARCA (anti-reverse CAP analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

Fragments of proteins: “Fragments” of proteins or peptides in the context of the present invention may, typically, comprise a sequence of a protein or peptide as defined herein, which is, with regard to its amino acid sequence (or its encoded nucleic acid molecule), N-terminally and/or C-terminally truncated compared to the amino acid sequence of the original (native) protein (or its encoded nucleic acid molecule). Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide.

Fragments of proteins or peptides in the context of the present invention may furthermore comprise a sequence of a protein or peptide as defined herein, which has a length of for example at least 5 amino acids, preferably a length of at least 6 amino acids, preferably at least 7 amino acids, more preferably at least 8 amino acids, even more preferably at least 9 amino acids; even more preferably at least 10 amino acids; even more preferably at least 11 amino acids; even more preferably at least 12 amino acids; even more preferably at least 13 amino acids; even more preferably at least 14 amino acids; even more preferably at least 15 amino acids; even more preferably at least 16 amino acids; even more preferably at least 17 amino acids; even more preferably at least 18 amino acids; even more preferably at least 19 amino acids; even more preferably at least 20 amino acids; even more preferably at least 25 amino acids; even more preferably at least 30 amino acids; even more preferably at least 35 amino acids; even more preferably at least 50 amino acids; or most preferably at least 100 amino acids. For example such fragment may have a length of about 6 to about 20 or even more amino acids, e.g. fragments as processed and presented by MHC class I molecules, preferably having a length of about 8 to about 10 amino acids, e.g. 8, 9, or 10, (or even 6, 7, 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T-cells in form of a complex consisting of the peptide fragment and an MHC molecule, i.e. the fragments are typically not recognized in their native form. Fragments of proteins or peptides may comprise at least one epitope of those proteins or peptides. Furthermore also domains of a protein, like the extracellular domain, the intracellular domain or the transmembrane domain and shortened or truncated versions of a protein may be understood to comprise a fragment of a protein.

Variants of proteins: “Variants” of proteins or peptides as defined in the context of the present invention may be generated, having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property. “Variants” of proteins or peptides as defined in the context of the present invention may comprise conservative amino acid substitution(s) compared to their native, i.e. non-mutated physiological, sequence. Those amino acid sequences as well as their encoding nucleotide sequences in particular fall under the term variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, for example, an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam).

A “variant” of a protein or peptide may have at least 70%, 75%, 80%, 85%, 90%, 95%, 98% or 99% amino acid identity over a stretch of 10, 20, 30, 50, 75 or 100 amino acids of such protein or peptide.

Furthermore, variants of proteins or peptides as defined herein, which may be encoded by a nucleic acid molecule, may also comprise those sequences, wherein nucleotides of the encoding nucleic acid sequence are exchanged according to the degeneration of the genetic code, without leading to an alteration of the respective amino acid sequence of the protein or peptide, i.e. the amino acid sequence or at least part thereof may not differ from the original sequence in one or more mutation(s) within the above meaning.

Identity of a sequence: In order to determine the percentage to which two sequences are identical, e.g. nucleic acid sequences or amino acid sequences as defined herein, preferably the amino acid sequences encoded by a nucleic acid sequence of the polymeric carrier as defined herein or the amino acid sequences themselves, the sequences can be aligned in order to be subsequently compared to one another. Therefore, e.g. a position of a first sequence may be compared with the corresponding position of the second sequence. If a position in the first sequence is occupied by the same component (residue) as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using a mathematical algorithm. A preferred, but not limiting, example of a mathematical algorithm which can be used is the algorithm of Karlin et al. (1993), PNAS USA, 90:5873-5877 or Altschul et al. (1997), Nucleic Acids Res., 25:3389-3402. Such an algorithm is integrated in the BLAST program. Sequences which are identical to the sequences of the present invention to a certain extent can be identified by this program.

Derivative of a protein or peptide: A derivative of a peptide or protein is typically understood to be a molecule that is derived from another molecule, such as said peptide or protein. A “derivative” of a peptide or protein also encompasses fusions comprising a peptide or protein used in the present invention. For example, the fusion comprises a label, such as, for example, an epitope, e.g., a FLAG epitope or a V5 epitope. For example, the epitope is a FLAG epitope. Such a tag is useful for, for example, purifying the fusion protein.

Monocistronic mRNA: A monocistronic mRNA may typically be an mRNA, that encodes only one open reading frame. An open reading frame in this context is a sequence of several nucleotide triplets (codons) that can be translated into a peptide or protein.

Nucleic acid: The term nucleic acid means any DNA- or RNA-molecule and is used synonymous with polynucleotide. Wherever herein reference is made to a nucleic acid or nucleic acid sequence encoding a particular protein and/or peptide, said nucleic acid or nucleic acid sequence, respectively, preferably also comprises regulatory sequences allowing in a suitable host, e.g. a human being, its expression, i.e. transcription and/or translation of the nucleic acid sequence encoding the particular protein or peptide.

Peptide: A peptide is a polymer of amino acid monomers. Usually the monomers are linked by peptide bonds. The term “peptide” does not limit the length of the polymer chain of amino acids. In some embodiments of the present invention a peptide may for example contain less than 50 monomer units. Longer peptides are also called polypeptides, typically having 50 to 600 monomeric units, more specifically 50 to 300 monomeric units.

Pharmaceutically effective amount: A pharmaceutically effective amount in the context of the invention is typically understood to be an amount that is sufficient to induce an immune response.

Protein: A protein typically consists of one or more peptides and/or polypeptides folded into 3-dimensional form, facilitating a biological function.

Poly (C) sequence: A poly-(C)-sequence is typically a long sequence of cytosine nucleotides, typically about 10 to about 200 cytosine nucleotides, preferably about 10 to about 100 cytosine nucleotides, more preferably about 10 to about 70 cytosine nucleotides or even more preferably about 20 to about 50 or even about 20 to about 30 cytosine nucleotides. A poly(C) sequence may preferably be located 3′ of the coding region comprised by a nucleic acid.

Poly-A-tail: A poly-A-tail also called “3′-poly(A) tail” is typically a long sequence of adenosine nucleotides of up to about 400 adenosine nucleotides, e.g. from about 25 to about 400, preferably from about 50 to about 400, more preferably from about 50 to about 300, even more preferably from about 50 to about 250, most preferably from about 60 to about 250 adenosine nucleotides, added to the 3′ end of a RNA.

Stabilized nucleic acid: A stabilized nucleic acid, typically, exhibits a modification increasing resistance to in vivo degradation (e.g. degradation by an exo- or endo-nuclease) and/or ex vivo degradation (e.g. by the manufacturing process prior to vaccine administration, e.g. in the course of the preparation of the vaccine solution to be administered). Stabilization of RNA can, e.g., be achieved by providing a 5′-CAP-Structure, a Poly-A-Tail, or any other UTR-modification. It can also be achieved by backbone-modification or modification of the G/C-content of the nucleic acid. Various other methods are known in the art and conceivable in the context of the invention.

Carrier/polymeric carrier: A carrier in the context of the invention may typically be a compound that facilitates transport and/or complexation of another compound. Said carrier may form a complex with said other compound. A polymeric carrier is a carrier that is formed of a polymer.

Cationic component: The term “cationic component” typically refers to a charged molecule, which is positively charged (cation) at a pH value of typically about 1 to 9, preferably of a pH value of or below 9 (e.g. 5 to 9), of or below 8 (e.g. 5 to 8), of or below 7 (e.g. 5 to 7), most preferably at physiological pH values, e.g. about 7.3 to 7.4. Accordingly, a cationic peptide, protein or polymer according to the present invention is positively charged under physiological conditions, particularly under physiological salt conditions of the cell in vivo. A cationic peptide or protein preferably contains a larger number of cationic amino acids, e.g. a larger number of Arg, His, Lys or Orn than other amino acid residues (in particular more cationic amino acids than anionic amino acid residues like Asp or Glu) or contains blocks predominantly formed by cationic amino acid residues. The definition “cationic” may also refer to “polycationic” components.

Vehicle: An agent, e.g. a carrier, that may typically be used within a pharmaceutical composition or vaccine for facilitating administering of the components of the pharmaceutical composition or vaccine to an individual.

3′-untranslated region (3′-UTR): A 3′-UTR is typically the part of an mRNA which is located between the protein coding region (i.e. the open reading frame) and the poly(A) sequence of the mRNA. A 3′-UTR of the mRNA is not translated into an amino acid sequence. The 3′-UTR sequence is generally encoded by the gene which is transcribed into the respective mRNA during the gene expression process. The genomic sequence is first transcribed into pre-mature mRNA, which comprises optional introns. The pre-mature mRNA is then further processed into mature mRNA in a maturation process. This maturation process comprises the steps of 5′-Capping, splicing the pre-mature mRNA to excise optional introns and modifications of the 3′-end, such as polyadenylation of the 3′-end of the pre-mature mRNA and optional endo- or exonuclease cleavages etc. In the context of the present invention, a 3′-UTR corresponds to the sequence of a mature mRNA which is located 3′ to the stop codon of the protein coding region, preferably immediately 3′ to the stop codon of the protein coding region, and which extends to the 5′-side of the poly(A) sequence, preferably to the nucleotide immediately 5′ to the poly(A) sequence. The term “corresponds to” means that the 3′-UTR sequence may be an RNA sequence, such as in the mRNA sequence used for defining the 3′-UTR sequence, or a DNA sequence which corresponds to such RNA sequence. In the context of the present invention, the term “a 3′-UTR of a gene”, such as “a 3′-UTR of an albumin gene”, is the sequence which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the RNA sequence of the 3′-UTR.

5′-untranslated region (5′-UTR): A 5′-UTR is typically understood to be a particular section of messenger RNA (mRNA). It is located 5′ of the open reading frame of the mRNA. Typically, the 5′-UTR starts with the transcriptional start site and ends one nucleotide before the start codon of the open reading frame. The 5′-UTR may comprise elements for controlling gene expression, also called regulatory elements. Such regulatory elements may be, for example, ribosomal binding sites or a 5′-Terminal Oligopyrimidine Tract. The 5′-UTR may be posttranscriptionally modified, for example by addition of a 5′-CAP. In the context of the present invention, a 5′-UTR corresponds to the sequence of a mature mRNA which is located between the 5′-CAP and the start codon. Preferably, the 5′-UTR corresponds to the sequence which extends from a nucleotide located 3′ to the 5′-CAP, preferably from the nucleotide located immediately 3′ to the 5′-CAP, to a nucleotide located 5′ to the start codon of the protein coding region, preferably to the nucleotide located immediately 5′ to the start codon of the protein coding region. The nucleotide located immediately 3′ to the 5′-CAP of a mature mRNA typically corresponds to the transcriptional start site. The term “corresponds to” means that the 5′-UTR sequence may be an RNA sequence, such as in the mRNA sequence used for defining the 5′-UTR sequence, or a DNA sequence which corresponds to such RNA sequence. In the context of the present invention, the term “a 5′-UTR of a gene”, such as “a 5′-UTR of a TOP gene”, is the sequence which corresponds to the 5′-UTR of the mature mRNA derived from this gene, i.e. the MRNA obtained by transcription of the gene and maturation of the pre-mature mRNA. The term “5′-UTR of a gene” encompasses the DNA sequence and the RNA sequence of the 5′-UTR.

5′Terminal Oligopyrimidine Tract (TOP): The 5′terminal oligopyrimidine tract (TOP) is typically a stretch of pyrimidine nucleotides located at the 5′ terminal region of a nucleic acid molecule, such as the 5′ terminal region of certain mRNA molecules or the 5′ terminal region of a functional entity, e.g. the transcribed region, of certain genes. The sequence starts with a cytidine, which usually corresponds to the transcriptional start site, and is followed by a stretch of usually about 3 to 30 pyrimidine nucleotides. For example, the TOP may comprise 3, 4, 5, 6, 7, 8, 9, 10, 11, 12, 13, 14, 15, 16, 17, 18, 19, 20, 21, 22, 23, 24, 25, 26, 27, 28, 29, 30 or even more nucleotides. The pyrimidine stretch and thus the 5′ TOP ends one nucleotide 5′ to the first purine nucleotide located downstream of the TOP. Messenger RNA that contains a 5′terminal oligopyrimidine tract is often referred to as TOP mRNA. Accordingly, genes that provide such messenger RNAs are referred to as TOP genes. TOP sequences have, for example, been found in genes and mRNAs encoding peptide elongation factors and ribosomal proteins.

TOP motif: In the context of the present invention, a TOP motif is a nucleic acid sequence which corresponds to a 5′TOP as defined above. Thus, a TOP motif in the context of the present invention is preferably a stretch of pyrimidine nucleotides having a length of 3-30 nucleotides. Preferably, the TOP-motif consists of at least 3 pyrimidine nucleotides, preferably at least 4 pyrimidine nucleotides, preferably at least 5 pyrimidine nucleotides, more preferably at least 6 nucleotides, more preferably at least 7 nucleotides, most preferably at least 8 pyrimidine nucleotides, wherein the stretch of pyrimidine nucleotides preferably starts at its 5′end with a cytosine nucleotide. In TOP genes and TOP mRNAs, the TOP-motif preferably starts at its 5′end with the transcriptional start site and ends one nucleotide 5′ to the first purin residue in said gene or mRNA. A TOP motif in the sense of the present invention is preferably located at the 5′end of a sequence which represents a 5′-UTR or at the 5′end of a sequence which codes for a 5′-UTR. Thus, preferably, a stretch of 3 or more pyrimidine nucleotides is called “TOP motif” in the sense of the present invention if this stretch is located at the 5′end of a respective sequence, such as the inventive mRNA, the 5′-UTR element of the inventive mRNA, or the nucleic acid sequence which is derived from the 5′-UTR of a TOP gene as described herein. In other words, a stretch of 3 or more pyrimidine nucleotides which is not located at the 5′-end of a 5′-UTR or a 5′-UTR element but anywhere within a 5′-UTR or a 5′-UTR element is preferably not referred to as “TOP motif”.

TOP gene: TOP genes are typically characterised by the presence of a 5′ terminal oligopyrimidine tract. Furthermore, most TOP genes are characterized by a growth-associated translational regulation. However, also TOP genes with a tissue specific translational regulation are known. As defined above, the 5′-UTR of a TOP gene corresponds to the sequence of a 5′-UTR of a mature mRNA derived from a TOP gene, which preferably extends from the nucleotide located 3′ to the 5′-CAP to the nucleotide located 5′ to the start codon. A 5′-UTR of a TOP gene typically does not comprise any start codons, preferably no upstream AUGs (uAUGs) or upstream open reading frames (uORFs). Therein, upstream AUGs and upstream open reading frames are typically understood to be AUGs and open reading frames that occur 5′ of the start codon (AUG) of the open reading frame that should be translated. The 5′-UTRs of TOP genes are generally rather short. The lengths of 5′-UTRs of TOP genes may vary between 20 nucleotides up to 500 nucleotides, and are typically less than about 200 nucleotides, preferably less than about 150 nucleotides, more preferably less than about 100 nucleotides. Exemplary 5′-UTRs of TOP genes in the sense of the present invention are the nucleic acid sequences extending from the nucleotide at position 5 to the nucleotide located immediately 5′ to the start codon (e.g. the ATG) in the sequences according to SEQ ID Nos. 1-1363, SEQ ID NO. 1395, SEQ ID NO. 1421 and SEQ ID NO. 1422 of the international patent application WO2013/143700 or homologs or variants thereof, whose disclosure is incorporated herewith by reference. In this context a particularly preferred fragment of a 5′-UTR of a TOP gene is a 5′-UTR of a TOP gene lacking the 5′TOP motif. The term ‘5’-UTR of a TOP gene′ preferably refers to the 5′-UTR of a naturally occurring TOP gene.

Fragment of a nucleic acid sequence, particularly an mRNA: A fragment of a nucleic acid sequence consists of a continuous stretch of nucleotides corresponding to a continuous stretch of nucleotides in the full-length nucleic acid sequence which is the basis for the nucleic acid sequence of the fragment, which represents at least 20%, preferably at least 30%, more preferably at least 40%, more preferably at least 50%, even more preferably at least 60%, even more preferably at least 70%, even more preferably at least 80%, and most preferably at least 90% of the full-length nucleic acid sequence. Such a fragment, in the sense of the present invention, is preferably a functional fragment of the full-length nucleic acid sequence.

Variant of a nucleic acid sequence, particularly an mRNA: A variant of a nucleic acid sequence refers to a variant of nucleic acid sequences which forms the basis of a nucleic acid sequence. For example, a variant nucleic acid sequence may exhibit one or more nucleotide deletions, insertions, additions and/or substitutions compared to the nucleic acid sequence from which the variant is derived. Preferably, a variant of a nucleic acid sequence is at least 40%, preferably at least 50%, more preferably at least 60%, more preferably at least 70%, even more preferably at least 80%, even more preferably at least 90%, most preferably at least 95% identical to the nucleic acid sequence the variant is derived from. Preferably, the variant is a functional variant. A “variant” of a nucleic acid sequence may have at least 70%, 75%, 80%, 85%, 90%, 95%, 98% or 99% nucleotide identity over a stretch of 10, 20, 30, 50, 75 or 100 nucleotide of such nucleic acid sequence.

Homolog of a nucleic acid sequence: The term “homolog” of a nucleic acid sequence refers to sequences of other species than the particular sequence. It is particular preferred that the nucleic acid sequence is of human origin and therefore it is preferred that the homolog is a homolog of a human nucleic acid sequence.

Jet injection: The term “jet injection”, as used herein, refers to a needle-free injection method, wherein a fluid containing at least one inventive mRNA sequence and, optionally, further suitable excipients is forced through an orifice, thus generating an ultra-fine liquid stream of high pressure that is capable of penetrating mammalian skin and, depending on the injection settings, subcutaneous tissue or muscle tissue. In principle, the liquid stream forms a hole in the skin, through which the liquid stream is pushed into the target tissue. Preferably, jet injection is used for intradermal, subcutaneous or intramuscular injection of the mRNA sequence according to the invention. In a preferred embodiment, jet injection is used for intramuscular injection of the mRNA sequence according to the invention. In a further preferred embodiment, jet injection is used for intradermal injection of the mRNA sequence according to the invention.

The present invention is based on the inventors' surprising finding that an mRNA sequence comprising a coding region, encoding at least one antigenic peptide or protein derived from the glycoprotein (GP) and/or the matrix protein 40 (VP40) and/or the nucleoprotein (NP) of a virus of the genus Ebolavirus or Marburgvirus induces efficiently antigen-specific immune responses against Ebolaviruses or Marburgviruses.

Furthermore, the inventors surprisingly found that mRNA-based vaccines, like the mRNA-based Ebolavirus or Marburgvirus vaccine, according to the invention was biologically active after storage at 40° C. for 6 months and even after storage at 60ºC for 1 month. Therefore, the mRNA-based Ebolavirus or Marburgvirus vaccine according to the invention would be an attractive vaccine in developing countries, since it can be stored at ambient temperature. In summary, the inventive mRNA sequence comprising a coding region encoding at least one antigenic peptide or protein derived from the glycoprotein (GP) and/or the matrix protein 40 (VP40) and/or the nucleoprotein (NP) of a virus of the genus Ebolavirus or Marburgvirus could contribute to provide affordable, readily available, temperature-stable Ebolavirus or Marburgvirus vaccines, particularly for preexposure and postexposure of Ebolavirus or Marburgvirus prophylaxis for the developed and developing world.

Additionally, the mRNA sequence according to the invention enables rapid and rational vaccine design with flexibility, speed and scalability of production probably exceeding those of current virus-based technologies.

According to an especially preferred embodiment of the invention, the inventive mRNA is modified and thus stabilized by modifying and increasing the G (guanosine)/C (cytosine) content of the mRNA of the coding region thereof. Therein, the G/C content of the inventive mRNA of the coding region is increased compared to the G/C content of the coding region of its particular wild type coding sequence, i.e. the unmodified mRNA. However, the encoded amino acid sequence of the inventive mRNA is preferably not modified compared to the coded amino acid sequence of the particular wild type/unmodified mRNA.

The modification of the G/C-content of the inventive mRNA is based on the fact that RNA sequences having an increased G (guanosine)/C (cytosine) content are more stable than RNA sequences having an increased A (adenosine)/U (uracil) content. The codons of a coding sequence or a whole RNA might therefore be varied compared to the wild type coding sequence or mRNA, such that they include an increased amount of G/C nucleotides while the translated amino acid sequence is retained. In respect to the fact that several codons code for one and the same amino acid (so-called degeneration of the genetic code), the most favourable codons for the stability can be determined (so-called alternative codon usage). Preferably, the G/C content of the coding region of the inventive mRNA according to the invention is increased by at least 7%, more preferably by at least 15%, particularly preferably by at least 20%, compared to the G/C content of the coding region of the wild type RNA. According to a specific embodiment at least 5%, 10%, 20%, 30%, 40%, 50%, 60%, more preferably at least 70%, even more preferably at least 80% and most preferably at least 90%, 95% or even 100% of the substitutable codons in the region coding for a protein or peptide as defined herein or its fragment or variant thereof or the whole sequence of the wild type mRNA sequence or coding sequence are substituted, thereby increasing the G/C content of said sequence. In this context, it is particularly preferable to increase the G/C content of the inventive mRNA to the maximum (i.e. 100% of the substitutable codons), in particular in the coding region, compared to the wild type sequence.

Prior vaccines against Ebolavirus disease or Marburgvirus disease that have been developed at this point rely on different ways of transferring the antigen. Promising results have been generated using protein-based vaccines. However, protein-based vaccines are extremely expensive and time consuming in production. Given the high variability and fast infection rates of potential new outbreaks of Marburgviruses and Ebolaviruses, fast adjustments of a vaccine are possible using vaccines based on the present invention. Furthermore, given that outbreaks have so far mostly been restricted to developing countries, high production costs of a potential vaccine according to prior approaches might be problematic. In contrast, vaccines based on the present invention may be produced in a cost-efficient manner. Moreover, the vaccines based on the invention are much safer than e.g. DNA-based vaccines since RNA-based vaccines cannot permanently be integrated in the genome.

In a particularly preferred embodiment of the first aspect of the invention the inventive mRNA sequence comprises a coding region, encoding at least one antigenic peptide or protein derived from the glycoprotein (GP) of a virus of the genus Ebolavirus or Marburgvirus or a fragment, variant or derivative thereof (GP mRNA sequence(s)). It is especially preferred to combine at least one mRNA sequence encoding at least one antigenic peptide or protein derived from the glycoprotein (GP) with mRNA sequence(s) encoding at least one antigenic peptide or protein derived from the matrix protein 40 (VP40) and/or the nucleoprotein (NP) or fragments, variants or derivatives thereof. In certain embodiments, a RNA molecule comprises a 5′ UTR, an ORF (e.g., encoding GP, VP40 or NP from Ebolavirus or Marburgvirus) and 3′ UTR sequence wherein the 5′ UTR sequence or the 3′ UTR sequence is heterologous relative the ORF of the mRNA (e.g., wherein the RNA does not comprise the 5′ UTR sequence and/or the 3′ UTR sequence of a wild type RNA encoding the ORF).

Thus, in one embodiment, there is provided a method of providing an immune response in a subject comprising: (a) obtaining a RNA molecule encoding a Ebolavirus or Marburgvirus antigen (e.g., GP, VP40 or NP antigen); (b) storing the molecule for at least 1 month (e.g., 2, 3, 4, 5, 6 or more months), without the use of refrigeration; and (c) administering the RNA to a subject, thereby providing an immune response in the subject. In certain embodiments, an RNA molecule is stored at ambient temperature, such as between about 10° C. and 40° C. In further embodiments, the RNA is comprised in an aqueous solution during storage or is lyophilized.

In this context, the amino acid sequence of the at least one antigenic peptide or protein may be selected from any peptide or protein derived from the glycoprotein GP, the matrix protein VP40 and the nucleoprotein NP of any Ebolavirus or Marburgvirus isolate or a fragment, variant or derivative thereof or from any synthetically engineered Ebolavirus or Marburgvirus peptide or protein.

Glycoprotein (GP) is a viral surface protein which generally functions in host cell attachment and fusion. This protein, which represents the primary component on the viral surface, was chosen for an mRNA-based vaccine in order to effectively induce an immune response against Ebolavirus and/or Marburgvirus infections.

In a preferred embodiment of the present invention the coding region of the wild type mRNA encoding at least one peptide or protein derived from the glycoprotein (GP) includes an editing site of seven consecutive adenosine residues (A stretch), wherein one further adenosine residue ist added. The modified sequence including eight adenosine residues is taken as inventive mRNA sequence, wherein preferably the modified sequence is taken as basis for the modification of the G/C content as described above. The resulting mRNA sequence leads to expression of full length GP which is surprisingly most effective in inducing an immune response. It is known that the gp gene in Ebolaviruses encodes for three glycoproteins: a full-length 676 residue protein, GP, that represents the structural surface glycoprotein as described above, as well as two smaller secreted forms, i.e. sGP (364 residues) and ssGP (298 residues). The different forms are produced via a transcriptional editing site, a template sequence of seven consecutive uridine (U) residues that can lead to slippage of the viral polymerase. Transcripts containing the unedited seven adenines (A) encode for sGP, while a frameshift induced by the addition of an A in the transcribed mRNA leads to the overriding of a stop codon and therefore to the expression of full length GP. ssGP is produced via another frameshifted transcript by two additional adenosine residues at the editing site which are inserted during the transcriptional process (de La Vega M. et al. (2014), VIRAL IMMUNOLOGY, Vol. 28, no. 1, 1-7). EBOV transcripts are produced in a ratio of 24:71:5 (GP:sGP:ssGP) in Vero cell culture (Mehedi M. et al. (2011), Journal of Virology, 5406-5414). It was already shown by Wong G. et al. (Sci Transl Med. 2012 Oct. 31; 4(158)) that Glycoprotein specific IgG levels is a meaningful correlate of protection against Zaire ebolavirus in an animal model.

By the inventive approach and especially by using the above mentioned modification of the mRNA as basis for an mRNA vaccine it is possible to enable translation of full length GP in the patient inducing effectively an immune response. Especially for Ebolavirus GP, RNA-based vaccines according to the invention offer the additional advantage that modification of the editing site via G/C-optimisation abolish the 8A stretch and thus prevent polymerase slippage and the production of frameshifted proteins that lead to inefficient immune responses. Since the unmodified editing site leads to polymerase slippage even in systems in which enzymes other than the viral polymerase is used (Volchkov V. E. et al. (1995), Virology, Vol. 214, 421-430), all prior approaches, regardless of how the antigen is produced, might suffer from the problem unless the nucleotide sequence is modified. This would also be true for in vitro transcription systems comprising viral polymerases as T3, T7 or Sp6. This problem is solved by the inventive approach wherein the editing site is modified by insertion of an additional adenosine residue within the editing site and in an especially effective embodiment by modification and optimisation of the C/G content of the mRNA and especially of the coding region. Thereby the editing site is altered and slippage of polymerase is prevented. This enables a particularly effective way translation of full-length GP.

In contrast to Ebolaviruses, the gp gene in viruses belonging to the Marburgvirus genus lacks this editing site and therefore only encodes for one surface glycoprotein that functions in receptor binding and viral entry. The above described modification of the mRNA, namely insertion of a further adenosine residue in the editing site, relates solely to viruses of the genus Ebolavirus.

According to a preferred embodiment of the invention the mRNA sequence or a composition of mRNA sequences usable as an Ebolavirus or Marburgvirus vaccine includes at least one mRNA sequence comprising a coding region, encoding at least one antigenic peptide or protein derived from the matrix protein VP40 of a virus of the genus Ebolavirus or Marburgvirus or a fragment, variant or derivative thereof (VP40 mRNA sequence(s)). The combination of GP mRNA sequences with VP40 mRNA sequences results in an especially effective vaccine formulation. The matrix protein VP40 is known to be a viral structural protein that functions in assembly and budding of Filoviridae. VP40 is the most abundant protein in viral particles (40% molecular weight) and provides the basis for VLP (virus-like particles) formation: the protein alone is able to induce the formation and release of VLPs and, in doing so, is able to incorporate additional viral proteins (reviewed in Warfield K. L. and Aman M. J. (2011), JID, 204 (Suppl 3)). Moreover, VP40 contains both T- and B-cell epitopes. IgGs in asymptomatic patients have been reported to be mainly reactive to VP40 (Becquart P. et al. (2014), PLOS One, Vol. 9, no. 6: e96360). Thus the combination of GP mRNA sequences and VP40 mRNA sequences according to the invention is very effective in triggering an immune response in the patient.

According to a further preferred embodiment of the invention the mRNA sequence or a composition of mRNA sequences usable as an Ebolavirus or Marburgvirus vaccine includes at least one mRNA sequence comprising a coding region, encoding at least one antigenic peptide or protein derived from the nucleoprotein (NP) of a virus of the genus Ebolavirus or Marburgvirus or a fragment, variant or derivative thereof (NP mRNA sequence(s)). The combination of GP mRNA sequences with NP mRNA sequences results in an especially effective vaccine formulation. Viral nucleoprotein (NP) functions in the protection of the viral genome. It is known that NP contains T- and B-cell epitopes, wherein IgGs in asymptomatic patients have been reported to be reactive to NP (Leroy E. M. et al. (2000), Lancet., Vol. 355 (9222): 2210-5). Thus the combination of GP mRNA sequences and NP mRNA sequences, preferably in combination with VP40 mRNA sequences, according to the invention is particularly effective in triggering an immune response in the patient.

Sequences employed according to the present invention may include GP mRNA sequences and/or VP40 mRNA sequences and/or NP mRNA sequences from more than one, preferably of several Ebolavirus and/or Marburgvirus strains. It is especially preferred to combine different mRNAs encoding different glycoproteins and/or matrix proteins 40 and/or nucleoproteins in a multivalent vaccine because the combination will be especially effective to fully protect against a potential Ebolavirus or Marburgvirus outbreak.

In a particularly preferred embodiment the full-length protein of the glycoprotein (GP) and/or the matrix protein 40 (VP40) and/or the nucleoprotein (NP) is encoded by the coding region(s) comprised in the inventive mRNA sequence(s) or mRNA composition. With regard to GP mRNA sequences of Ebolaviruses, the full-length transcription is preferably achieved by modification and optimisation of the editing site as described above.

In a further particularly preferred embodiment a fragment comprising at least one epitope of the glycoprotein (GP) and/or the matrix protein 40 (VP40) and/or the nucleoprotein (NP) is encoded by the coding region(s) comprised in the inventive mRNA sequence(s) or mRNA composition.

In a preferred embodiment of the present invention the employed GP mRNA sequences and/or VP40 mRNA sequences and/or NP mRNA sequences are derived from the species Ebola ebolavirus (EBOV) and/or Bundibugyo ebolavirus (BDBV) and/or Sudan ebolavirus (SUDV) and/or Taï Forest ebolavirus (TAFV) and/or Marburg marburgvirus (MARV). Preferably the GP mRNA sequences and/or VP40 mRNA sequences and/or NP mRNA sequences encode EBOV amino acid sequences isolated in an outbreak from 1976 as well as from 2014 and/or SUDV amino acid sequences and/or BDBV amino acid sequences and/or TAFV amino acid sequences and/or MARV amino acid sequences.

The following preferred amino acid sequences may form the basis for the inventive mRNA.

The first amino acid sequence (SEQ ID NO. 1) refers to a glycoprotein (GP) of an Ebolavirus strain EBOV isolated in an outbreak from 1976 in Mayinga, Zaire. The sequence derives from NCBI identification AAD14585.1, GI:4262350 from complete genome AF086833.2, GI:10141003.

EBOV GP, Mayinga, Zaire 1976
Amino acid sequence (SEQ ID NO. 1):
MGVTGILQLPRDRFKRTSFFLWVIILFQRTFSIPLGVIHNSTLQVSDVDK
LVCRDKLSSTNQLRSVGLNLEGNGVATDVPSATKRWGFRSGVPPKVVNYE
AGEWAENCYNLEIKKPDGSECLPAAPDGIRGFPRCRYVHKVSGTGPCAGD
FAFHKEGAFFLYDRLASTVIYRGTTFAEGVVAFLILPQAKKDFFSSHPLR
EPVNATEDPSSGYYSTTIRYQATGFGTNETEYLFEVDNLTYVQLESRFTP
QFLLQLNETIYTSGKRSNTTGKLIWKVNPEIDTTIGEWAFWETKKNLTRK
IRSEELSFTVVSNGAKNISGQSPARTSSDPGTNTTTEDHKIMASENSSAM
VQVHSQGREAAVSHLTTLATISTSPQSLTTKPGPDNSTHNTPVYKLDISE
ATQVEQHHRRTDNDSTASDTPSATTAAGPPKAENTNTSKSTDFLDPATTT
SPQNHSETAGNNNTHHQDTGEESASSGKLGLITNTIAGVAGLITGGRRTR
REAIVNAQPKCNPNLHYWTTQDEGAAIGLAWIPYFGPAAEGIYIEGLMHN
QDGLICGLRQLANETTQALQLFLRATTELRTFSILNRKAIDFLLQRWGGT
CHILGPDCCIEPHDWTKNITDKIDQIIHDFVDKTLPDQGDNDNWWTGWRQ
WIPAGIGVTGVIIAVIALFCICKFVF

The following preferred amino acid sequence (SEQ ID NO. 2) refers to a glycoprotein of an Ebolavirus strain EBOV isolated in an outbreak from 1976 in Sierra Leone. The sequence derives from NCBI identification AIG96616.1; GI:667853336 from complete sequence Genbank: KM233116.1, originally published in Science 12 Sep. 2014: vol. 345 no. 6202: 1369-1372.

EBOV GP, Sierra Leone 2014
Amino acid sequence (SEQ ID NO. 2):
MGVTGILQLPRDRFKRTSFFLWVIILFQRTFSIPLGVIHNSTLQVSDVDK
LVCRDKLSSTNQLRSVGLNLEGNGVATDVPSVTKRWGFRSGVPPKVVNYE
AGEWAENCYNLEIKKPDGSECLPAAPDGIRGFPRCRYVHKVSGTGPCAGD
FAFHKEGAFFLYDRLASTVIYRGTTFAEGVVAFLILPQAKKDFFSSHPLR
EPVNATEDPSSGYYSTTIRYQATGFGTNETEYLFEVDNLTYVQLESRFTP
QFLLQLNETIYASGKRSNTTGKLIWKVNPEIDTTIGEWAFWETKKNLTRK
IRSEELSFTAVSNGPKNISGQSPARTSSDPETNTTNEDHKIMASENSSAM
VQVHSQGRKAAVSHLTTLATISTSPQPPTTKTGPDNSTHNTPVYKLDISE
ATQVGQHHRRADNDSTASDTPPATTAAGPLKAENTNTSKSADSLDLATTT
SPQNYSETAGNNNTHHQDTGEESASSGKLGLITNTIAGVAGLITGGRRTR
REVIVNAQPKCNPNLHYWTTQDEGAAIGLAWIPYFGPAAEGIYTEGLMHN
QDGLICGLRQLANETTQALQLFLRATTELRTFSILNRKAIDFLLQRWGGT
CHILGPDCCIEPHDWTKNITDKIDQIIHDFVDKTLPDQGDNDNWWTGWRQ
WIPAGIGVTGVIIAVIALFCICKFVF

The following preferred amino acid sequence (SEQ ID NO. 3) refers to a glycoprotein of a Marburgvirus strain MARV isolated in Angola in 2005. The sequence derives from NCBI identification ABE27015.1; GI:91177683 from complete sequence GenBank: DQ447653.1.

MARV GP, Angola 2005
Amino acid sequence (SEQ ID NO. 3):
MKTTCLLISLILIQGVKTLPILEIASNIQPQNVDSVCSGTLQKTEDVHLM
GFTLSGQKVADSPLEASKRWAFRAGVPPKNVEYTEGEEAKTCYNISVTDP
SGKSLLLDPPTNIRDYPKCKTIHHIQGQNPHAQGIALHLWGAFFLYDRIA
STTMYRGKVFTEGNIAAMIVNKTVHKMIFSRQGQGYRHMNLTSTNKYWTS
SNGTQTNDTGCFGTLQEYNSTKNQTCAPSKKPLPLPTAHPEVKLTSTSTD
ATKLNTTDPNSDDEDLTTSGSGSGEQEPYTTSDAATKQGLSSTMPPTPSP
QPSTPQQGGNNTNHSQGVVTEPGKTNTTAQPSMPPHNTTTISTNNTSKHN
LSTPSVPIQNATNYNTQSTAPENEQTSAPSKTTLLPTENPTTAKSTNSTK
SPTTTVPNTTNKYSTSPSPTPNSTAQHLVYFRRKRNILWREGDMFPFLDG
LINAPIDFDPVPNTKTIFDESSSSGASAEEDQHASPNISLTLSYFPKVNE
NTAHSGENENDCDAELRIWSVQEDDLAAGLSWIPFFGPGIEGLYTAGLIK
NQNNLVCRLRRLANQTAKSLELLLRVTTEERTFSLINRHAIDFLLARWGG
TCKVLGPDCCIGIEDLSRNISEQIDQIKKDEQKEGTGWGLGGKWWTSDWG
VLTNLGILLLLSIAVLIALSCICRIFTKYIG

The following preferred amino acid sequence (SEQ ID NO. 4) refers to a glycoprotein of an Ebolavirus strain BDBV isolated in Uganda in 2007. The sequence derives from NCBI identification ACI28624.1, GI 208436390 from complete sequence FJ217161.1, GI:208436385.

BDBV GP, Uganda 2007
Amino acid sequence (SEQ ID NO. 4):
MVTSGILQLPRERFRKTSFFVWVIILFHKVFPIPLGWHNNTLQVSDIDKL
VCRDKLSSTSQLKSVGLNLEGNGVATDVPTATKRWGFRAGVPPKVVNYEA
GEWAENCYNLDIKKADGSECLPEAPEGVRGFPRCRYVHKVSGTGPCPEGY
AFHKEGAFFLYDRLASTIIYRSTTFSEGVVAFLILPETKKDFFQSPPLHE
PANMTTDPSSYYHTVTLNYVADNFGTNMTNFLFQVDHLTYVQLEPRFTPQ
FLVQLNETIYTNGRRSNTTGTLIWKVNPTVDTGVGEWAFWENKKNFTKTL
SSEELSVIFVPRAQDPGSNQKTKVTPTSFANNQTSKNHEDLVPEDPASVV
QVRDLQRENTVPTPPPDTVPTTLIPDTMEEQTTSHYEPPNISRNHQERNN
TAHPETLANNPPDNTTPSTPPQDGERTSSHTTPSPRPVPTSTIHPTTRET
HIPTTMTTSHDTDSNRPNPIDISESTEPGPLTNTTRGAANLLTGSRRTRR
EITLRTQAKCNPNLHYWTTQDEGAAIGLAWIPYFGPAAEGIYTEGIMHNQ
NGLICGLRQLANETTQALQLFLRATTELRTFSILNRKAIDFLLQRWGGTC
HILGPDCCIEPHDWTKNITDKIDQIIHDFIDKPLPDQTDNDNWWTGWRQW
VPAGIGITGVIIAVIALLCICKFLL

The following preferred amino acid sequence (SEQ ID NO. 5) refers to a glycoprotein of an Ebolavirus strain SUDV isolated in Uganda in 2000. The sequence derives from NCBI Identification Q7T9D9.1, GI:75559166 from complete sequence NC_006432.1, GI:55770807.

SUDV GP, Gulu, Uganda 2007
Amino acid sequence (SEQ ID NO. 5):
MGGLSLLQLPRDKFRKSSFFVWVIILFQKAFSMPLGVVTNSTLEVTEIDQ
LVCKDHLASTDQLKSVGLNLEGSGVSTDIPSATKRWGFRSGVPPKVVSYE
AGEWAENCYNLEIKKPDGSECLPPPPDGVRGFPRCRYVHKAQGTGPCPGD
YAFHKDGAFFLYDRLASTVIYRGVNFAEGVIAFLILAKPKETFLQSPPIR
EAVNYTENTSSYYATSYLEYEIENFGAQHSTTLFKIDNNTFVRLDRPHTP
QFLFQLNDTIHLHQQLSNTTGRLIWTLDANINADIGEWAFWENKKNLSEQ
LRGEELSFEALSLNETEDDDAASSRITKGRISDRATRKYSDLVPKNSPGM
VPLHIPEGETTLPSQNSTEGRRVGVNTQETITETAATIIGTNGNHMQIST
IGIRPSSSQIPSSSPTTAPSPEAQTPTTHTSGPSVMATEEPTTPPGSSPG
PTTEAPTLTTPENITTAVKTVLPQESTSNGLITSTVTGILGSLGLRKRSR
RQTNTKATGKCNPNLHYWTAQEQHNAAGIAWIPYFGPGAEGIYTEGLMHN
QNALVCGLRQLANETTQALQLFLRATTELRTYTILNRKAIDFLLRRWGGT
CRILGPDCCIEPHDWTKNITDKINQIIHDFIDNPLPNQDNDDNWWTGWRQ
WIPAGIGITGIIIAIIALLCVCKLLC

The following preferred amino acid sequence (SEQ ID NO. 6) refers to a glycoprotein of an Ebolavirus strain TAFV isolated in Cote d′Ivoire in 1994. The sequence derives from NCBI Identification YP_003815426.1. GI: 302315373 from complete sequence NC_014372.1, GI:302315369.

TAFV GP, Cote d'lvoire 1994
Amino acid sequence (SEQ ID NO. 6):
MGASGILQLPRERFRKTSFFVWVIILFHKVFSIPLGVVHNNTLQVSDIDK
FVCRDKLSSTSQLKSVGLNLEGNGVATDVPTATKRWGFRAGVPPKVVNCE
AGEWAENCYNLAIKKVDGSECLPEAPEGVRDFPRCRYVHKVSGTGPCPGG
LAFHKEGAFFLYDRLASTIIYRGTTFAEGVIAFLILPKARKDFFQSPPLH
EPANMTTDPSSYYHTTTINYVVDNFGTNTTEFLFQVDHLTYVQLEARFTP
QFLVLLNETIYSDNRRSNTTGKLIWKINPTVDTSMGEWAFWENKKNFTKT
LSSEELSFVPVPETQNQVLDTTATVSPPISAHNHAAEDHKELVSEDSTPV
VQMQNIKGKDTMPTTVTGVPTTTPSPFPINARNTDHTKSFIGLEGPQEDH
STTQPAKTTSQPTNSTESTTLNPTSEPSSRGTGPSSPTVPNTTESHAELG
KTTPTTLPEQHTAASAIPRAVHPDELSGPGFLTNTIRGVTNLLTGSRRKR
RDVTPNTQPKCNPNLHYWTALDEGAAIGLAWIPYFGPAAEGIYTEGIMEN
QNGLICGLRQLANETTQALQLFLRATTELRTFSILNRKAIDFLLQRWGGT
CHILGPDCCIEPQDWTKNITDKIDQIIHDFVDNNLPNQNDGSNWWTGWKQ
WVPAGIGITGVIIAIIALLCICKFML

The following preferred amino acid sequence (SEQ ID NO. 7) refers to a matrix protein VP40 of an Ebolavirus strain EBOV isolated in Mayinga, Zaire in 1976. The sequence derives from NCBI identification ID AAD14583.1, GI:4262348 from complete genome AF086833.2 GI:10141003.

EBOV VP40, Mayinga, Zaire 1976
Amino acid sequence (SEQ ID NO. 7):
MRRVILPTAPPEYMEAIYPVRSNSTIARGGNSNTGFLTPESVNGDTPSNP
LRPIADDTIDHASHTPGSVSSAFILEAMVNVISGPKVLMKQIPIWLPLGV
ADQKTYSFDSTTAAIMLASYTITHFGKATNPLVRVNRLGPGIPDHPLRLL
RIGNQAFLQEFVLPPVQLPQYFTFDLTALKLITQPLPAATWTDDTPTGSN
GALRPGISFHPKLRPILLPNKSGKKGNSADLTSPEKIQAIMTSLQDFKIV
PIDPTKNIMGIEVPETLVHKLTGKKVTSKNGQPIIPVLLPKYIGLDPVAP
GDLTMVITQDCDTCHSPASLPAVIEK

The following preferred amino acid sequence (SEQ ID NO. 8) refers to a matrix protein VP40 of an Ebolavirus strain EBOV isolated in Sierra Leone in 2014. The sequence derives from NCBI identification AIG96615.1, GI:667853335 from complete sequence GenBank: KM233116.1.

EBOV VP40, Sierra Leone 2014
Amino acid sequence (SEQ ID NO. 8):
MRRVILPTAPPEYMEAIYPARSNSTIARGGNSNTGFLTPESVNGDTPSNP
LRPIADDTIDHASHTPGSVSSAFILEAMVNVISGPKVLMKQIPIWLPLGV
ADQKTYSFDSTTAAIMLASYTITHFGKATNPLVRVNRLGPGIPDHPLRLL
RIGNQAFLQEFVLPPVQLPQYFTFDLTALKLITQPLPAATWTDDTPTGSN
GALRPGISFHPKLRPILLPNKSGKKGNSADLTSPEKIQAIMTSLQDFKIV
PIDPTKNIMGIEVPETLVHKLTGKKVTSKNGQPIIPVLLPKYIGLDPVAP
GDLTMVITQDCDTCHSPASLPAVVEK

The following preferred amino acid sequence (SEQ ID NO. 9) refers to a matrix protein VP40 of a Marburgvirus strain MARV isolated in Angola in 2005. The sequence derives from NCBI identification protein_id ABE27014.1, GI:91177682 from complete sequence GenBank:DQ447653.1.

MARV VP40, Angola 2005
Amino acid sequence (SEQ ID NO. 9):
MASSSNYNTYMQYLNPPPYADHGANQLIPADQLSNQQGITPNYVGDLNLD
DQFKGNVCHAFTLEAIIDISAYNERTVKGVPAWLPLGIMSNFEYPLAHTV
AALLTGSYTITQFTHNGQKFVRVNRLGTGIPAHPLRMLREGNQAFIQNMV
IPRNFSTNQFTYNLTNLVLSVQKLPDDAWRPSKDKLIGNTMHPAVSVHPN
LPPIVLPTVKKQAYRQHKNPNNGPLLAISGILHQLRVEKVPEKTSLFRIS
LPADMFSVKEGMMKKRGENSPVVYFQAPENFPLNGFNNRQVVLAYANPTL
SAV

The following preferred amino acid sequence (SEQ ID NO. 10) refers to a matrix protein VP40 of an Ebolavirus strain BDBV isolated in Uganda in 2007. The sequence derives from NCBI identification ACI28622.1, GI 208436388 from complete sequence FJ217161.1, GI:208436385.

BDBV VP40, Uganda 2007
Amino acid sequence (SEQ ID NO. 10):
MRRAILPTAPPEYIEAVYPMRTVSTSINSTASGPNFPAPDVMMSDTPSNS
LRPIADDNIDHPSHTPTSVSSAFILEAMVNVISGPKVLMKQIPIWLPLGV
ADQKTYSFDSTTAAIMLASYTITHFGKTSNPLVRINRLGPGIPDHPLRLL
RIGNQAFLQEFVLPPVQLPQYFTFDLTALKLITQPLPAATWTDDTPTGPT
GILRPGISFHPKLRPILLPGKTGKRGSSSDLTSPDKIQAIMNFLQDLKLV
PIDPAKNIMGIEVPELLVHRLTGKKITTKNGQPIIPILLPKYIGMDPISQ
GDLTMVITQDCDTCHSPASLPPVSEK

The following preferred amino acid sequence (SEQ ID NO. 11) refers to a matrix protein VP40 of an Ebolavirus strain SUDV isolated in Uganda in 2000. The sequence derives from NCBI identification YP_138522.1, GI: 55770810 from complete sequence NC_006432.1, GI:55770807.

SUDV VP40, Gulu, Uganda 2000
Amino acid sequence (SEQ ID NO. 11):
MRRVTVPTAPPAYADIGYPMSMLPIKSSRAVSGIQQKQEVLPGMDTPSNS
MRPVADDNIDHTSHTPNGVASAFILEATVNVISGPKVLMKQIPIWLPLGI
ADQKTYSFDSTTAAIMLASYTITHFGKANNPLVRVNRLGQGIPDHPLRLL
RMGNQAFLQEFVLPPVQLPQYFTFDLTALKLVTQPLPAATWTDETPSNLS
GALRPGLSFHPKLRPVLLPGKTGKKGHVSDLTAPDKIQTIVNLMQDFKIV
PIDPAKSIIGIEVPELLVHKLTGKKMSQKNGQPIIPVLLPKYIGLDPISP
GDLTMVITPDYDDCHSPASCSYLSEK

The following preferred amino acid sequence (SEQ ID NO. 12) refers to a matrix protein VP40 of an Ebolavirus strain TAFV isolated in Cote d′Ivoire in 1994. The sequence derives from NCBI identification YP_003815425.1, GI: 302315372 from complete sequence NC_014372.1 GI:302315369.

TAFV VP40, Cote d'lvoire 1994
Amino acid sequence (SEQ ID NO. 12):
MRRIILPTAPPEYMEAVYPMRTMNSGADNTASGPNYTTTGVMTNDTPSNS
LRPVADDNIDHPSHTPNSVASAFILEAMVNVISGPKVLMKQIPIWLPLGV
SDQKTYSFDSTTAAIMLASYTITHFGKTSNPLVRINRLGPGIPDHPLRLL
RIGNQAFLQEFVLPPVQLPQYFTFDLTALKLITQPLPAATWTDETPAVST
GTLRPGISFHPKLRPILLPGRAGKKGSNSDLTSPDKIQAIMNFLQDLKIV
PIDPTKNIMGIEVPELLVHRLTGKKTTTKNGQPIIPILLPKYIGLDPLSQ
GDLTMVITQDCDSCHSPASLPPVNEK

The following preferred amino acid sequence (SEQ ID NO. 13) refers to a nucleoprotein NP of an Ebolavirus strain EBOV isolated in Mayinga, Zaire in 1976. The sequence derives from NCBI identification AAD14590.1, GI:4262355 from complete genome AF086833.2 GI:10141003.

EBOV NP, Mayinga, Zaire 1976
Amino acid sequence (SEQ ID NO. 13):
MDSRPQKIWMAPSLTESDMDYHKILTAGLSVQQGIVRQRVIPVYQVNNLE
EICQLIIQAFEAGVDFQESADSFLLMLCLHHAYQGDYKLFLESGAVKYLE
GHGFRFEVKKRDGVKRLEELLPAVSSGKNIKRTLAAMPEEETTEANAGQF
LSFASLFLPKLVVGEKACLEKVQRQIQVHAEQGLIQYPTAWQSVGHMMVI
FRLMRTNFLIKFLLIHQGMHMVAGHDANDAVISNSVAQARFSGLLIVKTV
LDHILQKTERGVRLHPLARTAKVKNEVNSFKAALSSLAKHGEYAPFARLL
NLSGVNNLEHGLFPQLSAIALGVATAHGSTLAGVNVGEQYQQLREAATEA
EKQLQQYAESRELDHLGLDDQEKKILMNFHQKKNEISFQQTNAMVTLRKE
RLAKLTEAITAASLPKTSGHYDDDDDIPFPGPINDDDNPGHQDDDPTDSQ
DTTIPDVVVDPDDGSYGEYQSYSENGMNAPDDLVLFDLDEDDEDTKPVPN
RSTKGGQQKNSQKGQHIEGRQTQSRPIQNVPGPHRTIHHASAPLTDNDRR
NEPSGSTSPRMLTPINEEADPLDDADDETSSLPPLESDDEEQDRDGTSNR
TPTVAPPAPVYRDHSEKKELPQDEQQDQDHTQEARNQDSDNTQSEHSFEE
MYRHILRSQGPFDAVLYYHMMKDEPVVFSTSDGKEYTYPDSLEEEYPPWL
TEKEAMNEENRFVTLDGQQFYWPVMNHKNKFMAILQHHQ

The following preferred amino acid sequence (SEQ ID NO. 14) refers to a nucleoprotein NP of an Ebolavirus strain EBOV isolated in Sierra Leone in 2014. The sequence derives from NCBI identification AIG96613.1, GI:667853333 from complete sequence GenBank: KM233116.1.

EBOV NP, Sierra Leone 2014
Amino acid sequence (SEQ ID NO. 14):
MDSRPQKVWMTPSLTESDMDYHKILTAGLSVQQGIVRQRVIPVYQVNNLE
EICQLIIQAFEAGVDFQESADSFLLMLCLHHAYQGDYKLFLESGAVKYLE
GHGFRFEVKKCDGVKRLEELLPAVSSGRNIKRTLAAMPEEETTEANAGQF
LSFASLFLPKLVVGEKACLEKVQRQIQVHAEQGLIQYPTAWQSVGHMMVI
FRLMRTNFLIKFLLIHQGMHMVAGHDANDAVISNSVAQARFSGLLIVKTV
LDHILQKTERGVRLHPLARTAKVKNEVNSFKAALSSLAKHGEYAPFARLL
NLSGVNNLEHGLFPQLSAIALGVATAHGSTLAGVNVGEQYQQLREAATEA
EKQLQQYAESRELDHLGLDDQEKKILMNFHQKKNEISFQQTNAMVTLRKE
RLAKLTEAITAASLPKTSGHYDDDDDIPFPGPINDDDNPGHQDDDPTDSQ
DTTIPDVVVDPDDGGYGEYQSYSENGMSAPDDLVLFDLDEDDEDTKPVPN
RSTKGGQQKNSQKGQHTEGRQTQSTPTQNVTGPRRTIHHASAPLTDNDRR
NEPSGSTSPRMLTPINEEADPLDDADDETSSLPPLESDDEEQDRDGTSNR
TPTVAPPAPVYRDHSEKKELPQDEQQDQDHIQEARNQDSDNTQPEHSFEE
MYRHILRSQGPFDAVLYYHMMKDEPVVFSTSDGKEYTYPDSLEEEYPPWL
TEKEAMNDENRFVTLDGQQFYWPVMNHRNKFMAILQHHQ

The following preferred amino acid sequence (SEQ ID NO. 15) refers to a nucleoprotein NP of a Marburgvirus strain MARV isolated in Angola in 2005. The sequence derives from NCBI identification ABE27012.1, GI:91177680 from complete sequence GenBank:DQ447653.1.

MARV NP, Angola 2005
Amino acid sequence (SEQ ID NO. 15):
MDLHSLLELGTKPTAPHVRNKKVILFDTNHQVSICNQIIDAINSGIDLGD
LLEGGLLTLCVEHYYNSDKDKFNTSPIAKYLRDAGYEFDVIKNADATRFL
DVIPNEPHYSPLILALKTLESTESQRGRIGLFLSFCSLFLPKLVVGDRAS
IEKALRQVTVHQEQGIVTYPNHWLTTGHMKVIFGILRSSFILKFVLIHQG
VNLVTGHDAYDSIISNSVGQTRFSGLLIVKTVLEFILQKTDSGVTLHPLV
RTSKVKNEVASFKQALSNLARHGEYAPFARVLNLSGINNLEHGLYPQLSA
IALGVATAHGSTLAGVNVGEQYQQLREAAHDAEVKLQRRHEHQEIQAIAE
DDEERKILEQFHLQKTEITHSQTLAVLSQKREKLARLAAEIENNIVEDQG
FKQSQNRVSQSFLNDPTPVEVTVQARPINRPTALPPPVDSKIEHESTEDS
SSSSSFVDLNDPFALLNEDEDTLDDSVMIPSTTSREFQGIPEPPRQSQDI
DNSQGKQEDESTNLIKKPFLRYQELPPVQEDDESEYTTDSQESIDQPGSD
NEQGVDLPPPPLYAQEKRQDPIQHPAVSSQDPFGSIGDVNGDILEPIRSP
SSPSAPQEDTRAREAYELSPDFTNYEDNQQNWPQRVVTKKGRTFLYPNDL
LQTNPPESLITALVEEYQNPVSAKELQADWPDMSFDERRHVAMNL

The following preferred amino acid sequence (SEQ ID NO. 16) refers to a nucleoprotein NP of an Ebolavirus strain BDBV isolated in Uganda in 2007. The sequence derives from NCBI identification ACI28620.1, GI 208436386 from complete sequence FJ217161.1, GI:208436385.

BDBV NP, Uganda 2007
Amino acid sequence (SEQ ID NO. 16):
MDPRPIRTWMMHNTSEVEADYHKILTAGLSVQQGIVRQRIIPVYQISNLE
EVCQLIIQAFEAGVDFQDSADSFLLMLCLHHAYQGDYKQFLESNAVKYLE
GHGFRFEMKKKEGVKRLEELLPAASSGKNIKRTLAAMPEEETTEANAGQF
LSFASLFLPKLVVGEKACLEKVQRQIQVHAEQGLIQYPTSWQSVGHMMVI
FRLMRTNFLIKFLLIHQGMHMVAGHDANDAVIANSVAQARFSGLLIVKTV
LDHILQKTEHGVRLHPLARTAKVKNEVSSFKAALASLAQHGEYAPFARLL
NLSGVNNLEHGLFPQLSAIALGVATAHGSTLAGVNVGEQYQQLREAATEA
EKQLQKYAESRELDHLGLDDQEKKILKDFHQKKNEISFQQTTAMVTLRKE
RLAKLTEAITSTSILKTGRRYDDDNDIPFPGPINDNENSGQNDDDPTDSQ
DTTIPDVIIDPNDGGYNNYSDYANDAASAPDDLVLFDLEDEDDADNPAQN
TPEKNDRPATTKLRNGQDQDGNQGETASPRVAPNQYRDKPMPQVQDRSEN
HDQTLQTQSRVLTPISEEADPSDHNDGDNESIPPLESDDEGSTDTTAAET
KPATAPPAPVYRSISVDDSVPSENIPAQSNQTNNEDNVRNNAQSEQSIAE
MYQHILKTQGPFDAILYYHMMKEEPIIFSTSDGKEYTYPDSLEDEYPPWL
SEKEAMNEDNRFITMDGQQFYWPVMNHRNKFMAILQHHR

The following preferred amino acid sequence (SEQ ID NO. 17) refers to a nucleoprotein NP of an Ebolavirus strain SUDV isolated in Uganda in 2000. The sequence derives from NCBI identification YP_138520.1, GI 55770808 from complete sequence NC_006432.1, GI:55770807.

SUDV NP, Gulu, Uganda 2000
Amino acid sequence (SEQ ID NO. 17):
MDKRVRGSWALGGQSEVDLDYHKILTAGLSVQQGIVRQRVIPVYWSDLEG
ICQHIIQAFEAGVDFQDNADSFLLLLCLHHAYQGDHRLFLKSDAVQYLEG
HGFRFEVREKENVHRLDELLPNVTGGKNLRRTLAAMPEEETTEANAGQFL
SFASLFLPKLVVGEKACLEKVQRQIQVHAEQGLIQYPTSWQSVGHMMVIF
RLMRTNFLIKFLLIHQGMHMVAGHDANDTVISNSVAQARFSGLLIVKTVL
DHILQKTDLGVRLHPLARTAKVKNEVSSFKAALGSLAKHGEYAPFARLLN
LSGVNNLEHGLYPQLSAIALGVATAHGSTLAGVNVGEQYQQLREAATEAE
KQLQQYAETRELDNLGLDEQEKKILMSFHQKKNEISFQQTNAMVTLRKER
LAKLTEAITTASKIKVGDRYPDDNDIPFPGPIYDETHPNPSDDNPDDSRD
TTIPGGVVDPYDDESNNYPDYEDSAEGTTGDLDLFNLDDDDDDSQPGPPD
RGQSKERAARTHGLQDPTLDGAKKVPELTPGSHQPGNLHITKPGSNTNQP
QGNMSSTLQSMTPIQEESEPDDQKDDDDESLTSLDSEGDEDVESVSGENN
PTVAPPAPVYKDTGVDTNQQNGPSNAVDGQGSESEALPINPEKGSALEET
YYHLLKTQGPFEAINYYHLMSDEPIAFSTESGKEYIFPDSLEEAYPPWLS
EKEALEKENRYLVIDGQQFLWPVMSLQDKFLAVLQHD

The following preferred amino acid sequence (SEQ ID NO. 18) refers to a nucleoprotein NP of an Ebolavirus strain TAFV isolated in Cote d′Ivoire in 1994. The sequence derives from NCBI identification YP_003815423.1, GI: 302315370 from complete sequence NC_014372.1 GI:302315369.

TAFV NP, Cote d'lvoire 1994
Amino acid sequence (SEQ ID NO. 18):
MESRAHKAWMTHTASGFETDYHKILTAGLSVQQGIVRQRVIQVHQVTNLE
EICQLIIQAFEAGVDFQESADSFLLMLCLHHAYQGDYKQFLESNAVKYLE
GHGFRFEVRKKEGVKRLEELLPAASSGKSIRRTLAAMPEEETTEANAGQF
LSFASLFLPKLVVGEKACLEKVQRQIQVHSEQGLIQYPTAWQSVGHMMVI
FRLMRTNFLIKFLLIHQGMHMVAGHDANDAVIANSVAQARFSGLLIVKTV
LDHILQKTEHGVRLHPLARTAKVKNEVNSFKAALSSLAQHGEYAPFARLL
NLSGVNNLEHGLFPQLSAIALGVATAHGSTLAGVNVGEQYQQLREAATEA
EKQLQKYAESRELDHLGLDDQEKKILKDFHQKKNEISFQQTTAMVTLRKE
RLAKLTEAITSTSLLKTGKQYDDDNDIPFPGPINDNENSEQQDDDPTDSQ
DTTIPDIIVDPDDGRYNNYGDYPSETANAPEDLVLFDLEDGDEDDHRPSS
SSENNNKHSLTGTDSNKTSNWNRNPTNMPKKDSTQNNDNPAQRAQEYARD
NIQDTPTPHRALTPISEETGSNGHNEDDIDSIPPLESDEENNTETTITTT
KNTTAPPAPVYRSNSEKEPLPQEKSQKQPNQVSGSENTDNKPHSEQSVEE
MYRHILQTQGPFDAILYYYMMTEEPIVFSTSDGKEYVYPDSLEGEHPPWL
SEKEALNEDNRFITMDDQQFYWPVMNHRNKFMAILQHHK

In the context of the invention additionally to the here disclosed amino acid sequences according to SEQ ID Nos. 1-18 also amino acid sequences of different Ebolavirus or Marburgvirus isolates can be used according to the invention and are incorporated herewith. These different Ebolavirus or Marburgvirus isolates show preferably an identity of at least 70%, more preferably of at least 80% and most preferably of at least 90% with the amino acid sequences according to SEQ ID Nos. 1-18.

Furthermore, in this context the coding region encoding at least one antigenic peptide or protein derived from the glycoprotein (GP) and/or the matrix protein 40 (VP40) and/or the nucleoprotein (NP) of a virus of the genus Ebolavirus or Marburgvirus or a fragment, variant or derivative thereof, may be selected from any nucleic acid sequence comprising a coding region derived from any Ebolavirus or Marburgvirus isolate or a fragment or variant thereof.

Particularly preferred are the following nucleotide sequences encoding the glycoprotein (GP) and/or the matrix protein 40 (VP40) and/or the nucleoprotein (NP) of Ebolavirus or Marburgvirus species respectively the mRNA sequences corresponding to the following nucleotide sequences. With regard to GP mRNA sequences of Ebolavirus species, it is especially preferred to modify the sequences by insertion of an additional adenosine residue within the editing site of seven adenosine nucleotides of the wild type mRNA sequence resulting in a modified editing site of eight adenosine nucleotides. The following mRNA sequences according to SEQ ID NO. 20 and 21, which correspond to amino acid sequences according to SEQ ID Nos. 1 and 2, are modified in this way, wherein the information on the nucleotide sequence, derived from NCBI was amended by insertion of:

Insertion nucleotide sequence

(SEQ ID NO. 19)

ACCTCACTAGAAAAATTCGCAGTGAAGAGTTGTCTTTC

The insertion sequence was inserted in position 886-924 of the following sequences according to SEQ ID Nos. 20 and 21. The insertion sequence (SEQ ID NO. 19) is derived from a different EBOV sequence (AY354458.1). By this insertion the stretch of seven adenosine nucleotides (editing site) in position 880-886 is modified and comprises eight adenosine nucleotides (pos. 880-887). The resulting protein sequence (SEQ ID Nos. 1 and 2), namely full-length GP, remains unaltered. The resulting nucleotide sequences are termed modified wild type nucleotide sequences.

According to a preferred embodiment, the inventive mRNA comprises a coding region encoding at least one antigenic peptide or protein derived from a glycoprotein of Ebolavirus, preferably a glycoprotein comprising the amino acid sequence according to SEQ ID NO. 1 or 2, or a fragment, variant or derivative thereof, wherein an editing site, which preferably comprises seven adenosine nucleotides, was modified, preferably by insertion of an additional adenine residue, preferably immediately 5′ of a nuclei acid sequence comprising seven adenosine residues. According to a particularly preferred embodiment, a nucleic acid sequence corresponding to SEQ ID NO. 19 is inserted immediately 5′ of an editing site, preferably an editing site comprising seven adenosine nucleotides. According to one embodiment, the inventive mRNA thus comprises a coding region encoding a glycoprotein of Ebolavirus, preferably a glycoprotein comprising the amino acid sequence according to SEQ ID NO. 1 or 2, or a fragment, variant or derivative thereof, wherein the coding sequence comprises a nucleic acid sequence corresponding to SEQ ID NO. 19. More preferably, the inventive mRNA comprises a coding region encoding a glycoprotein of Ebolavirus, preferably a glycoprotein comprising the amino acid sequence according to SEQ ID NO. 1 or 2, or a fragment, variant or derivative thereof, wherein the coding sequence comprises a nucleic acid sequence corresponding to SEQ ID NO. 20, 21, 37, 38, 45, 46, 53, 54, 71, 72, 89, 90, 107, 108, 125, 126, 143, 144, 161, 162, 179, 180, 197, 198, 215 or 216. Alternatively, the inventive mRNA comprises a coding region encoding a glycoprotein of Ebolavirus, preferably a glycoprotein comprising the amino acid sequence according to SEQ ID NO. 1 or 2, or a fragment, variant or derivative thereof, wherein the coding sequence comprises a nucleic acid sequence having at least 80%, more preferably at least 85%, 90%, 95% or 99%, identity with SEQ ID NO. 20, 21, 37, 38, 45, 46, 53, 54, 71, 72, 89, 90, 107, 108, 125, 126, 143, 144, 161, 162, 179, 180, 197, 198, 215 or 216

The following modified wild type nucleotide sequence according to SEQ ID NO. 20 corresponds to the amino acid sequence according to SEQ ID NO. 1 and refers to the glycoprotein of an Ebolavirus strain EBOV isolated in an outbreak from 1976 in Mayinga, Zaire as described above. The insertion sequence according to SEQ ID NO. 19 is shown in italic, the modified editing site is shown in bold.

EBOV GP, Mayinga, Zaire 1976
Modified wild type nucleotide sequence of the
coding region (SEQ ID NO. 20):
ATGGGCGTTACAGGAATATTGCAGTTACCTCGTGATCGATTCAAGAGGAC
ATCATTCTTTCTTTGGGTAATTATCCTTTTCCAAAGAACATTTTCCATCC
CACTTGGAGTCATCCACAATAGCACATTACAGGTTAGTGATGTCGACAAA
CTAGTTTGTCGTGACAAACTGTCATCCACAAATCAATTGAGATCAGTTGG
ACTGAATCTCGAAGGGAATGGAGTGGCAACTGACGTGCCATCTGCAACTA
AAAGATGGGGCTTCAGGTCCGGTGTCCCACCAAAGGTGGTCAATTATGAA
GCTGGTGAATGGGCTGAAAACTGCTACAATCTTGAAATCAAAAAACCTGA
CGGGAGTGAGTGTCTACCAGCAGCGCCAGACGGGATTCGGGGCTTCCCCC
GGTGCCGGTATGTGCACAAAGTATCAGGAACGGGACCGTGTGCCGGAGAC
TTTGCCTTCCATAAAGAGGGTGCTTTCTTCCTGTATGATCGACTTGCTTC
CACAGTTATCTACCGAGGAACGACTTTCGCTGAAGGTGTCGTTGCATTTC
TGATACTGCCCCAAGCTAAGAAGGACTTCTTCAGCTCACACCCCTTGAGA
GAGCCGGTCAATGCAACGGAGGACCCGTCTAGTGGCTACTATTCTACCAC
AATTAGATATCAGGCTACCGGTTTTGGAACCAATGAGACAGAGTACTTGT
TCGAGGTTGACAATTTGACCTACGTCCAACTTGAATCAAGATTCACACCA
CAGTTTCTGCTCCAGCTGAATGAGACAATATATACAAGTGGGAAAAGGAG
CAATACCACGGGAAAACTAATTTGGAAGGTCAACCCCGAAATTGATACAA
CAATCGGGGAGTGGGCCTTCTGGGAAACTAAAAAAAACCTCACTAGAAAA
ATTCGCAGTGAAGAGTTGTCTTTCACAGTTGTATCAAACGGAGCCAAAAA
CATCAGTGGTCAGAGTCCGGCGCGAACTTCTTCCGACCCAGGGACCAACA
CAACAACTGAAGACCACAAAATCATGGCTTCAGAAAATTCCTCTGCAATG
GTTCAAGTGCACAGTCAAGGAAGGGAAGCTGCAGTGTCGCATCTAACAAC
CCTTGCCACAATCTCCACGAGTCCCCAATCCCTCACAACCAAACCAGGTC
CGGACAACAGCACCCATAATACACCCGTGTATAAACTTGACATCTCTGAG
GCAACTCAAGTTGAACAACATCACCGCAGAACAGACAACGACAGCACAGC
CTCCGACACTCCCTCTGCCACGACCGCAGCCGGACCCCCAAAAGCAGAGA
ACACCAACACGAGCAAGAGCACTGACTTCCTGGACCCCGCCACCACAACA
AGTCCCCAAAACCACAGCGAGACCGCTGGCAACAACAACACTCATCACCA
AGATACCGGAGAAGAGAGTGCCAGCAGCGGGAAGCTAGGCTTAATTACCA
ATACTATTGCTGGAGTCGCAGGACTGATCACAGGCGGGAGAAGAACTCGA
AGAGAAGCAATTGTCAATGCTCAACCCAAATGCAACCCTAATTTACATTA
CTGGACTACTCAGGATGAAGGTGCTGCAATCGGACTGGCCTGGATACCAT
ATTTCGGGCCAGCAGCCGAGGGAATTTACATAGAGGGGCTAATGCACAAT
CAAGATGGTTTAATCTGTGGGTTGAGACAGCTGGCCAACGAGACGACTCA
AGCTCTTCAACTGTTCCTGAGAGCCACAACTGAGCTACGCACCTTTTCAA
TCCTCAACCGTAAGGCAATTGATTTCTTGCTGCAGCGATGGGGCGGCACA
TGCCACATTCTGGGACCGGACTGCTGTATCGAACCACATGATTGGACCAA
GAACATAACAGACAAAATTGATCAGATTATTCATGATTTTGTTGATAAAA
CCCTTCCGGACCAGGGGGACAATGACAATTGGTGGACAGGATGGAGACAA
TGGATACCGGCAGGTATTGGAGTTACAGGCGTTATAATTGCAGTTATCGC
TTTATTCTGTATATGCAAATTTGTCTTTTAG

The following modified wild type nucleotide sequence according to SEQ ID NO. 21 corresponds to the amino acid sequence according to SEQ ID NO. 2 and refers to the glycoprotein of an Ebolavirus strain EBOV isolated in an outbreak from 2014 in Sierra Leone as described above. The insertion sequence according to SEQ ID NO. 19 is shown in italic, the modified editing site is shown in bold.

EBOV GP, Sierra Leone 2014
Modified wild type nucleotide sequence of the
coding region (SEQ ID NO. 21):
ATGGGTGTTACAGGAATATTGCAGTTACCTCGTGATCGATTCAAGAGGAC
ATCATTCTTTCTTTGGGTAATTATCCTTTTCCAAAGAACATTTTCCATCC
CGCTTGGAGTTATCCACAATAGTACATTACAGGTTAGTGATGTCGACAAA
CTAGTTTGTCGTGACAAACTGTCATCCACAAATCAATTGAGATCAGTTGG
ACTGAATCTCGAGGGGAATGGAGTGGCAACTGACGTGCCATCTGTGACTA
AAAGATGGGGCTTCAGGTCCGGTGTCCCACCAAAGGTGGTCAATTATGAA
GCTGGTGAATGGGCTGAAAACTGCTACAATCTTGAAATCAAAAAACCTGA
CGGGAGTGAGTGTCTACCAGCAGCGCCAGACGGGATTCGGGGCTTCCCCC
GGTGCCGGTATGTGCACAAAGTATCAGGAACGGGACCATGTGCCGGAGAC
TTTGCCTTCCACAAAGAGGGTGCTTTCTTCCTGTATGATCGACTTGCTTC
CACAGTTATCTACCGAGGAACGACTTTCGCTGAAGGTGTCGTTGCATTTC
TGATACTGCCCCAAGCTAAGAAGGACTTCTTCAGCTCACACCCCTTGAGA
GAGCCGGTCAATGCAACGGAGGACCCGTCGAGTGGCTATTATTCTACCAC
AATTAGATATCAGGCTACCGGTTTTGGAACTAATGAGACAGAGTACTTGT
TCGAGGTTGACAATTTGACCTACGTCCAACTTGAATCAAGATTCACACCA
CAGTTTCTGCTCCAGCTGAATGAGACAATATATGCAAGTGGGAAGAGGAG
CAACACCACGGGAAAACTAATTTGGAAGGTCAACCCCGAAATTGATACAA
CAATCGGGGAGTGGGCCTTCTGGGAAACTAAAAAAAACCTCACTAGAAAA
ATTCGCAGTGAAGAGTTGTCTTTCACAGCTGTATCAAACGGACCCAAAAA
CATCAGTGGTCAGAGTCCGGCGCGAACTTCTTCCGACCCAGAGACCAACA
CAACAAATGAAGACCACAAAATCATGGCTTCAGAAAATTCCTCTGCAATG
GTTCAAGTGCACAGTCAAGGAAGGAAAGCTGCAGTGTCGCATCTGACAAC
CCTTGCCACAATCTCCACGAGTCCTCAACCTCCCACAACCAAAACAGGTC
CGGACAACAGCACCCATAATACACCCGTGTATAAACTTGACATCTCTGAG
GCAACTCAAGTTGGACAACATCACCGTAGAGCAGACAACGACAGCACAGC
CTCCGACACTCCCCCCGCCACGACCGCAGCCGGACCCTTAAAAGCAGAGA
ACACCAACACGAGTAAGAGCGCTGACTCCCTGGACCTCGCCACCACGACA
AGCCCCCAAAACTACAGCGAGACTGCTGGCAACAACAACACTCATCACCA
AGATACCGGAGAAGAGAGTGCCAGCAGCGGGAAGCTAGGCTTAATTACCA
ATACTATTGCTGGAGTAGCAGGACTGATCACAGGCGGGAGAAGGACTCGA
AGAGAAGTAATTGTCAATGCTCAACCCAAATGCAACCCCAATTTACATTA
CTGGACTACTCAGGATGAAGGTGCTGCAATCGGATTGGCCTGGATACCAT
ATTTCGGGCCAGCAGCCGAAGGAATTTACACAGAGGGGCTAATGCACAAC
CAAGATGGTTTAATCTGTGGGTTGAGGCAGCTGGCCAACGAAACGACTCA
AGCTCTCCAACTGTTCCTGAGAGCCACAACTGAGCTGCGAACCTTTTCAA
TCCTCAACCGTAAGGCAATTGACTTCCTGCTGCAGCGATGGGGTGGCACA
TGCCACATTTTGGGACCGGACTGCTGTATCGAACCACATGATTGGACCAA
GAACATAACAGACAAAATTGATCAGATTATTCATGATTTTGTTGATAAAA
CCCTTCCGGACCAGGGGGACAATGACAATTGGTGGACAGGATGGAGACAA
TGGATACCGGCAGGTATTGGAGTTACAGGTGTTATAATTGCAGTTATCGC
TTTATTCTGTATATGCAAATTTGTCTTTTAG

The following wild type nucleotide sequence according to SEQ ID NO. 22 corresponds to the amino acid sequence according to SEQ ID NO. 3 and refers to the glycoprotein of a Marburgvirus strain MARV isolated in Angola in 2005 as described above.

MARV GP, Angola 2005
Wild type nucleotide sequence of the coding
region (SEQ ID NO. 22):
ATGAAAACCACATGTCTCCTTATCAGTCTTATCTTAATCCAAGGGGTAAA
AACTCTCCCTATTTTAGAGATAGCCAGTAACATTCAACCCCAAAATGTGG
ATTCAGTATGCTCCGGGACTCTCCAGAAGACAGAAGACGTTCATCTGATG
GGATTCACACTGAGCGGGCAAAAAGTTGCTGATTCCCCTTTAGAGGCATC
CAAACGATGGGCCTTCAGGGCAGGTGTACCTCCCAAGAATGTTGAGTATA
CAGAAGGGGAGGAAGCTAAAACATGTTACAATATAAGTGTAACGGATCCC
TCTGGAAAATCCTTGCTGTTAGATCCTCCTACCAACATCCGTGACTATCC
TAAATGCAAAACTATCCATCATATTCAAGGTCAAAACCCTCATGCACAGG
GGATCGCTCTCCATTTGTGGGGAGCATTTTTCTTGTATGATCGCATCGCC
TCCACAACGATGTATCGAGGCAAAGTCTTCACTGAAGGGAACATAGCAGC
TATGATTGTCAATAAGACAGTGCACAAAATGATTTTCTCGAGGCAAGGAC
AAGGGTACCGTCACATGAACCTAACTTCTACTAATAAATATTGGACAAGT
AGCAACGGAACGCAAACGAATGACACTGGATGCTTCGGTACTCTTCAAGA
ATATAATTCTACAAAGAACCAAACATGTGCTCCGTCCAAAAAACCTTTAC
CACTGCCCACAGCCCATCCGGAGGTCAAGCTCACTAGCACCTCAACTGAT
GCCACCAAACTCAATACCACAGACCCAAACAGTGATGATGAGGACCTCAC
AACATCTGGCTCAGGGTCTGGAGAACAGGAACCTTACACAACTTCTGACG
CAGCCACGAAGCAAGGGCTTTCATCAACAATGCCGCCCACTCCCTCACCA
CAACCAAGCACGCCACAGCAAGGAGGAAACAACACGAACCATTCCCAAGG
TGTTGTGACTGAACCCGGCAAAACCAACACAACTGCACAACCGTCCATGC
CCCCTCACAACACTACTACAATCTCTACTAACAACACCTCCAAGCACAAC
CTCAGCACTCCCTCTGTACCAATACAAAATGCCACTAATTACAACACACA
GAGCACGGCCCCTGAAAATGAGCAAACCAGTGCCCCCTCGAAAACAACCC
TGCTTCCAACAGAAAATCCTACAACAGCAAAGAGCACCAATAGTACAAAA
AGCCCCACTACAACAGTACCAAATACGACAAATAAGTATTCCACCAGTCC
CTCCCCCACCCCCAACTCGACTGCACAACATCTTGTATATTTCAGAAGGA
AACGAAATATTCTCTGGAGGGAAGGCGACATGTTCCCTTTTCTGGATGGG
TTAATAAATGCTCCGATTGATTTTGATCCGGTTCCAAATACAAAGACAAT
CTTTGATGAATCCTCTAGTTCTGGTGCTTCAGCTGAGGAAGATCAGCATG
CCTCCCCTAATATCAGTTTAACTTTATCTTACTTTCCTAAGGTAAATGAA
AACACTGCCCACTCTGGAGAAAATGAAAATGATTGTGATGCAGAGTTAAG
AATTTGGAGTGTTCAGGAGGACGACCTGGCAGCAGGACTCAGTTGGATAC
CGTTTTTTGGCCCTGGAATCGAAGGACTTTATACTGCTGGTTTAATTAAA
AATCAAAATAATTTGGTTTGCAGGTTGAGGCGTCTAGCCAATCAGACTGC
CAAATCCTTGGAACTCTTATTAAGAGTCACAACCGAGGAAAGAACATTTT
CCTTAATCAATAGACATGCCATTGATTTTTTACTCGCAAGGTGGGGAGGA
ACATGCAAAGTGCTTGGACCTGATTGTTGCATCGGAATAGAAGACTTGTC
CAGAAATATTTCAGAACAAATTGATCAAATCAAAAAGGACGAACAAAAAG
AGGGGACTGGTTGGGGTCTGGGTGGTAAATGGTGGACATCAGACTGGGGT
GTTCTTACTAACTTGGGCATCTTGCTACTACTGTCCATAGCTGTCTTAAT
TGCTCTGTCCTGTATTTGTCGTATTTTTACTAAATATATTGGATAA

The following wild type nucleotide sequence according to SEQ ID NO. 23 corresponds to the amino acid sequence according to SEQ ID NO. 7 and refers to the matrix protein VP40 of an Ebolavirus strain EBOV isolated in Zaire in 1976 as described above.

EBOV VP40, Mayinga, Zaire 1976
Wild type nucleotide sequence of the coding
region (SEQ ID NO. 23):
ATGAGGCGGGTTATATTGCCTACTGCTCCTCCTGAATATATGGAGGCCAT
ATACCCTGTCAGGTCAAATTCAACAATTGCTAGAGGTGGCAACAGCAATA
CAGGCTTCCTGACACCGGAGTCAGTCAATGGGGACACTCCATCGAATCCA
CTCAGGCCAATTGCCGATGACACCATCGACCATGCCAGCCACACACCAGG
CAGTGTGTCATCAGCATTCATCCTTGAAGCTATGGTGAATGTCATATCGG
GCCCCAAAGTGCTAATGAAGCAAATTCCAATTTGGCTTCCTCTAGGTGTC
GCTGATCAAAAGACCTACAGCTTTGACTCAACTACGGCCGCCATCATGCT
TGCTTCATACACTATCACCCATTTCGGCAAGGCAACCAATCCACTTGTCA
GAGTCAATCGGCTGGGTCCTGGAATCCCGGATCATCCCCTCAGGCTCCTG
CGAATTGGAAACCAGGCTTTCCTCCAGGAGTTCGTTCTTCCGCCAGTCCA
ACTACCCCAGTATTTCACCTTTGATTTGACAGCACTCAAACTGATCACCC
AACCACTGCCTGCTGCAACATGGACCGATGACACTCCAACAGGATCAAAT
GGAGCGTTGCGTCCAGGAATTTCATTTCATCCAAAACTTCGCCCCATTCT
TTTACCCAACAAAAGTGGGAAGAAGGGGAACAGTGCCGATCTAACATCTC
CGGAGAAAATCCAAGCAATAATGACTTCACTCCAGGACTTTAAGATCGTT
CCAATTGATCCAACCAAAAATATCATGGGAATCGAAGTGCCAGAAACTCT
GGTCCACAAGCTGACCGGTAAGAAGGTGACTTCTAAAAATGGACAACCAA
TCATCCCTGTTCTTTTGCCAAAGTACATTGGGTTGGACCCGGTGGCTCCA
GGAGACCTCACCATGGTAATCACACAGGATTGTGACACGTGTCATTCTCC
TGCAAGTCTTCCAGCTGTGATTGAGAAGTAA

The following wild type nucleotide sequence according to SEQ ID NO. 24 corresponds to the amino acid sequence according to SEQ ID NO. 8 and refers to the matrix protein VP40 of an Ebolavirus strain EBOV isolated in Sierra Leone in 2014 as described above.

EBOV VP40, Sierra Leone 2014
Wild type nucleotide sequence of the coding
region (SEQ ID NO. 24):
ATGAGGCGGGTTATATTGCCTACTGCTCCTCCTGAATATATGGAGGCCAT
ATACCCTGCCAGGTCAAATTCAACAATTGCTAGGGGTGGCAACAGCAATA
CAGGCTTCCTGACACCGGAGTCAGTCAATGGAGACACTCCATCGAATCCA
CTCAGGCCAATTGCTGATGACACCATCGACCATGCCAGCCACACACCAGG
CAGTGTGTCATCAGCATTCATCCTCGAAGCTATGGTGAATGTCATATCGG
GCCCCAAAGTGCTAATGAAGCAAATTCCAATTTGGCTTCCTCTAGGTGTC
GCTGATCAAAAGACCTACAGCTTTGACTCAACTACGGCCGCCATCATGCT
TGCTTCATATACTATCACCCATTTCGGCAAGGCAACCAATCCGCTTGTCA
GAGTCAATCGGCTGGGTCCTGGAATCCCGGATCACCCCCTCAGGCTCCTG
CGAATTGGAAACCAGGCTTTCCTCCAGGAGTTCGTTCTTCCACCAGTCCA
ACTACCCCAGTATTTCACCTTTGATTTGACAGCACTCAAACTGATCACTC
AACCACTGCCTGCTGCAACATGGACCGATGACACTCCAACTGGATCAAAT
GGAGCGTTGCGTCCAGGAATTTCATTTCATCCAAAACTTCGCCCCATTCT
TTTACCCAACAAAAGTGGGAAGAAGGGGAACAGTGCCGATCTAACATCTC
CGGAGAAAATCCAAGCAATAATGACTTCACTCCAGGACTTTAAGATCGTT
CCAATTGATCCAACCAAAAATATCATGGGTATCGAAGTGCCAGAAACTCT
GGTCCACAAGCTGACCGGTAAGAAGGTGACTTCCAAAAATGGACAACCAA
TCATCCCTGTTCTTTTGCCAAAGTACATTGGGTTGGACCCGGTGGCTCCA
GGAGACCTCACCATGGTAATCACACAGGATTGTGACACGTGTCATTCTCC
TGCAAGTCTTCCAGCTGTGGTTGAGAAGTAA

The following wild type nucleotide sequence according to SEQ ID NO. 25 corresponds to the amino acid sequence according to SEQ ID NO. 9 and refers to the matrix protein VP40 of a Marburgvirus strain MARV isolated in Angola in 2005 as described above.

MARV VP40, Angola 2005
Wild type nucleotide sequence of the coding
region (SEQ ID NO. 25):
ATGGCCAGTTCCAGCAATTACAATACATACATGCAATACCTTAACCCCCC
TCCTTATGCTGACCACGGTGCAAACCAGTTAATCCCGGCGGATCAGCTAT
CAAATCAGCAGGGTATAACTCCAAATTATGTGGGTGATTTAAACCTAGAT
GACCAGTTCAAAGGGAATGTCTGCCATGCTTTCACTTTAGAGGCAATAAT
TGACATATCTGCGTATAACGAACGAACAGTCAAAGGCGTTCCGGCATGGC
TGCCTCTTGGGATCATGAGCAATTTCGAATATCCTTTAGCCCATACAGTG
GCTGCGTTGCTCACAGGCAGCTATACAATCACCCAGTTTACTCATAATGG
GCAAAAATTCGTCCGTGTCAATCGACTCGGTACAGGAATCCCGGCACACC
CACTCAGGATGTTGCGTGAAGGAAATCAAGCTTTTATTCAGAATATGGTG
ATCCCCAGGAATTTTTCCACCAATCAATTCACCTACAATCTCACTAACTT
AGTATTGAGTGTGCAAAAACTTCCTGATGATGCCTGGCGTCCGTCCAAGG
ACAAATTAATTGGAAACACCATGCATCCTGCAGTCTCCGTTCACCCGAAT
TTACCGCCTATTGTTCTACCAACAGTCAAGAAGCAGGCTTATCGCCAGCA
CAAAAATCCCAACAATGGTCCACTGCTGGCCATATCTGGCATCCTTCATC
AACTGAGAGTCGAAAAAGTCCCAGAAAAGACAAGCCTGTTTAGGATTTCG
CTTCCTGCCGACATGTTCTCAGTAAAAGAGGGTATGATGAAGAAAAGAGG
AGAAAATTCCCCGGTAGTTTATTTTCAAGCACCTGAGAACTTCCCTTTGA
ATGGCTTCAACAACAGACAAGTTGTACTAGCGTATGCGAATCCAACACTC
AGCGCCGTTTAA

The following wild type nucleotide sequence according to SEQ ID NO. 26 corresponds to the amino acid sequence according to SEQ ID NO. 13 and refers to the nucleoprotein NP of an Ebolavirus strain EBOV isolated in Zaire in 1976 as described above.

EBOV NP, Zaire 1976
Wild type nucleotide sequence of the coding
region (SEQ ID NO. 26):
ATGGATTCTCGTCCTCAGAAAATCTGGATGGCGCCGAGTCTCACTGAATC
TGACATGGATTACCACAAGATCTTGACAGCAGGTCTGTCCGTTCAACAGG
GGATTGTTCGGCAAAGAGTCATCCCAGTGTATCAAGTAAACAATCTTGAA
GAAATTTGCCAACTTATCATACAGGCCTTTGAAGCAGGTGTTGATTTTCA
AGAGAGTGCGGACAGTTTCCTTCTCATGCTTTGTCTTCATCATGCGTACC
AGGGAGATTACAAACTTTTCTTGGAAAGTGGCGCAGTCAAGTATTTGGAA
GGGCACGGGTTCCGTTTTGAAGTCAAGAAGCGTGATGGAGTGAAGCGCCT
TGAGGAATTGCTGCCAGCAGTATCTAGTGGAAAAAACATTAAGAGAACAC
TTGCTGCCATGCCGGAAGAGGAGACAACTGAAGCTAATGCCGGTCAGTTT
CTCTCCTTTGCAAGTCTATTCCTTCCGAAATTGGTAGTAGGAGAAAAGGC
TTGCCTTGAGAAGGTTCAAAGGCAAATTCAAGTACATGCAGAGCAAGGAC
TGATACAATATCCAACAGCTTGGCAATCAGTAGGACACATGATGGTGATT
TTCCGTTTGATGCGAACAAATTTTCTGATCAAATTTCTCCTAATACACCA
AGGGATGCACATGGTTGCCGGGCATGATGCCAACGATGCTGTGATTTCAA
ATTCAGTGGCTCAAGCTCGTTTTTCAGGCTTATTGATTGTCAAAACAGTA
CTTGATCATATCCTACAAAAGACAGAACGAGGAGTTCGTCTCCATCCTCT
TGCAAGGACCGCCAAGGTAAAAAATGAGGTGAACTCCTTTAAGGCTGCAC
TCAGCTCCCTGGCCAAGCATGGAGAGTATGCTCCTTTCGCCCGACTTTTG
AACCTTTCTGGAGTAAATAATCTTGAGCATGGTCTTTTCCCTCAACTATC
GGCAATTGCACTCGGAGTCGCCACAGCACACGGGAGTACCCTCGCAGGAG
TAAATGTTGGAGAACAGTATCAACAACTCAGAGAGGCTGCCACTGAGGCT
GAGAAGCAACTCCAACAATATGCAGAGTCTCGCGAACTTGACCATCTTGG
ACTTGATGATCAGGAAAAGAAAATTCTTATGAACTTCCATCAGAAAAAGA
ACGAAATCAGCTTCCAGCAAACAAACGCTATGGTAACTCTAAGAAAAGAG
CGCCTGGCCAAGCTGACAGAAGCTATCACTGCTGCGTCACTGCCCAAAAC
AAGTGGACATTACGATGATGATGACGACATTCCCTTTCCAGGACCCATCA
ATGATGACGACAATCCTGGCCATCAAGATGATGATCCGACTGACTCACAG
GATACGACCATTCCCGATGTGGTGGTTGATCCCGATGATGGAAGCTACGG
CGAATACCAGAGTTACTCGGAAAACGGCATGAATGCACCAGATGACTTGG
TCCTATTCGATCTAGACGAGGACGACGAGGACACTAAGCCAGTGCCTAAT
AGATCGACCAAGGGTGGACAACAGAAGAACAGTCAAAAGGGCCAGCATAT
AGAGGGCAGACAGACACAATCCAGGCCAATTCAAAATGTCCCAGGCCCTC
ACAGAACAATCCACCACGCCAGTGCGCCACTCACGGACAATGACAGAAGA
AATGAACCCTCCGGCTCAACCAGCCCTCGCATGCTGACACCAATTAACGA
AGAGGCAGACCCACTGGACGATGCCGACGACGAGACGTCTAGCCTTCCGC
CCTTGGAGTCAGATGATGAAGAGCAGGACAGGGACGGAACTTCCAACCGC
ACACCCACTGTCGCCCCACCGGCTCCCGTATACAGAGATCACTCTGAAAA
GAAAGAACTCCCGCAAGACGAGCAACAAGATCAGGACCACACTCAAGAGG
CCAGGAACCAGGACAGTGACAACACCCAGTCAGAACACTCTTTTGAGGAG
ATGTATCGCCACATTCTAAGATCACAGGGGCCATTTGATGCTGTTTTGTA
TTATCATATGATGAAGGATGAGCCTGTAGTTTTCAGTACCAGTGATGGCA
AAGAGTACACGTATCCAGACTCCCTTGAAGAGGAATATCCACCATGGCTC
ACTGAAAAAGAGGCTATGAATGAAGAGAATAGATTTGTTACATTGGATGG
TCAACAATTTTATTGGCCGGTGATGAATCACAAGAATAAATTCATGGCAA
TCCTGCAACATCATCAGTGA

The following wild type nucleotide sequence according to SEQ ID NO. 27 corresponds to the amino acid sequence according to SEQ ID NO. 14 and refers to the nucleoprotein NP of an Ebolavirus strain EBOV isolated in Sierra Leone in 2014 as described above.

EBOV NP, Sierra Leone 2014
Wild type nucleotide sequence of the
coding region (SEQ ID NO. 27):
ATGGATTCTCGTCCTCAGAAAGTCTGGATGACGCCGAGTCTCACTGAATC
TGACATGGATTACCACAAGATCTTGACAGCAGGTCTGTCCGTTCAACAGG
GGATTGTTCGGCAAAGAGTCATCCCAGTGTATCAAGTAAACAATCTTGAG
GAAATTTGCCAACTTATCATACAGGCCTTTGAAGCTGGTGTTGATTTTCA
AGAGAGTGCGGACAGTTTCCTTCTCATGCTTTGTCTTCATCATGCGTACC
AAGGAGATTACAAACTTTTCTTGGAAAGTGGCGCAGTCAAGTATTTGGAA
GGGCACGGGTTCCGTTTTGAAGTCAAGAAGTGTGATGGAGTGAAGCGCCT
TGAGGAATTGCTGCCAGCAGTATCTAGTGGGAGAAACATTAAGAGAACAC
TTGCTGCCATGCCGGAAGAGGAGACGACTGAAGCTAATGCCGGTCAGTTC
CTCTCCTTTGCAAGTCTATTCCTTCCGAAATTGGTAGTAGGAGAAAAGGC
TTGCCTTGAGAAGGTTCAAAGGCAAATTCAAGTACATGCAGAGCAAGGAC
TGATACAATATCCAACAGCTTGGCAATCAGTAGGACACATGATGGTGATT
TTCCGTTTGATGCGAACAAATTTTTTGATCAAATTTCTTCTAATACACCA
AGGGATGCACATGGTTGCCGGACATGATGCCAACGATGCTGTGATTTCAA
ATTCAGTGGCTCAAGCTCGTTTTTCAGGTCTATTGATTGTCAAAACAGTA
CTTGATCATATCCTACAAAAGACAGAACGAGGAGTTCGTCTCCATCCTCT
TGCAAGGACCGCCAAGGTAAAAAATGAGGTGAACTCCTTCAAGGCTGCAC
TCAGCTCCCTGGCCAAGCATGGAGAGTATGCTCCTTTCGCCCGACTTTTG
AACCTTTCTGGAGTAAATAATCTTGAGCATGGTCTTTTCCCTCAACTGTC
GGCAATTGCACTCGGAGTCGCCACAGCCCACGGGAGCACCCTCGCAGGAG
TAAATGTTGGAGAACAGTATCAACAGCTCAGAGAGGCAGCCACTGAGGCT
GAGAAGCAACTCCAACAATATGCGGAGTCTCGTGAACTTGACCATCTTGG
ACTTGATGATCAGGAAAAGAAAATTCTTATGAACTTCCATCAGAAAAAGA
ACGAAATCAGCTTCCAGCAAACAAACGCGATGGTAACTCTAAGAAAAGAG
CGCCTGGCCAAGCTGACAGAAGCTATCACTGCTGCATCACTGCCCAAAAC
AAGTGGACATTACGATGATGATGACGACATTCCCTTTCCAGGACCCATCA
ATGATGACGACAATCCTGGCCATCAAGATGATGATCCGACTGACTCACAG
GATACGACCATTCCCGATGTGGTAGTTGACCCCGATGATGGAGGCTACGG
CGAATACCAAAGTTACTCGGAAAACGGCATGAGTGCACCAGATGACTTGG
TCCTATTCGATCTAGACGAGGACGACGAGGACACCAAGCCAGTGCCTAAC
AGATCGACCAAGGGTGGACAACAGAAAAACAGTCAAAAGGGCCAGCATAC
AGAGGGCAGACAGACACAATCCACGCCAACTCAAAACGTCACAGGCCCTC
GCAGAACAATCCACCATGCCAGTGCTCCACTCACGGACAATGACAGAAGA
AACGAACCCTCCGGCTCAACCAGCCCTCGCATGCTGACCCCAATCAACGA
AGAGGCAGACCCACTGGACGATGCCGACGACGAGACGTCTAGCCTTCCGC
CCTTAGAGTCAGATGATGAAGAACAGGACAGGGACGGAACTTCTAACCGC
ACACCCACTGTCGCCCCACCGGCTCCCGTATACAGAGATCACTCCGAAAA
GAAAGAACTCCCGCAAGATGAACAACAAGATCAGGACCACATTCAAGAGG
CCAGGAACCAAGACAGTGACAACACCCAGCCAGAACATTCTTTTGAGGAG
ATGTATCGCCACATTCTAAGATCACAGGGGCCATTTGATGCCGTTTTGTA
TTATCATATGATGAAGGATGAGCCTGTAGTTTTCAGTACCAGTGATGGTA
AAGAGTACACGTATCCGGACTCCCTTGAAGAGGAATATCCACCATGGCTC
ACTGAAAAAGAGGCCATGAATGATGAGAATAGATTTGTTACACTGGATGG
TCAACAATTTTATTGGCCAGTAATGAATCACAGGAATAAATTCATGGCAA
TCCTGCAACATCATCAGTGA

In the context of the invention additionally to the here disclosed nucleic acid sequences also nucleic acid sequences of different Ebolavirus or Marburgvirus isolates are incorporated herewith. These different virus isolates show preferably an identity of at least 50%, 60%, 70%, more preferably of at least 80% and most preferably of at least 90% with the nucleic acid sequences according to SEQ ID Nos. 20-27 or of fragments thereof.

The coding region of the inventive mRNA sequence according to the first aspect of the present invention may occur as a mono-, di-, or even multicistronic mRNA, i.e. an mRNA sequence which carries the coding sequences of one, two or more proteins or peptides. Such coding sequences in di-, or even multicistronic mRNAs may be separated by at least one internal ribosome entry site (IRES) sequence. For example, the internal ribosome entry site sequence may be derived vom EMCV (encephalomyocarditis virus) or from FMDV (Foot and mouth disease virus). Furthermore signal peptides may be used which induce the cleavage of the resulting polypeptide which comprises several proteins or peptides, e.g. a signal peptide sequence derived from F2A peptide from FMDV.

The following nucleotide sequence according to SEQ ID NO. 28 shows an example of an internal ribosome entry site of EMCV usable for the purposes of the present invention.

Nucleotide sequence of IRES of EMCV
(SEQ ID NO. 28)
TTGAAAGCCGGGGGTGGGAGATCCGGATTGCCAGTCTGCTCGATATCGCA
GGCTGGGTCCGTGACTACCCACTCCCCCTTTAATTCCGCCCCTCTCCCTC
CCCCCCCCCTAACGTTACTGGCCGAAGCCGCTTGGAATAAGGCCGGTGTG
CGTTTGTCTATATGTTATTTTCCACCATATTGCCGTCTTTTGGCAATGTG
AGGGCCCGGAAACCTGGCCCTGTCTTCTTGACGAGCATTCCTAGGGGTCT
TTCCCCTCTCGCCAAAGGAATGCAAGGTCTGTTGAATGTCGTGAAGGAAG
CAGTTCCTCTGGAAGCTTCTTGAAGACAAACAACGTCTGTAGCGACCCTT
TGCAGGCAGCGGAACCCCCCACCTGGCGACAGGTGCCTCTGCGGCCAAAA
GCCACGTGTATAAGATACACCTGCAAAGGCGGCACAACCCCAGTGCCACG
TTGTGAGTTGGATAGTTGTGGAAAGAGTCAAATGGCTCTCCTCAAGCGTA
TTCAACAAGGGGCTGAAGGATGCCCAGAAGGTACCCCATTGTATGGGATC
TGATCTGGGGCCTCGGTGCACATGCTTTACGTGTGTTTAGTCGAGGTTAA
AAAACGTCTAGGCCCCCCGAACCACGGGGACGTGGTTTTCCTTTGAAAAA
CACGATGATAATAGATCTACC

The following nucleotide sequence according to SEQ ID NO. 29 shows an example of an internal ribosome entry site of FMDV (GenBank: AJ133357.1, GI:6318187; 5′ UTR pos. 578-1038; point mutation 86 T→C from PMID: 8389904; point mutation 454 T→A; removal of first start codon pos. 454-456) usable for the purposes of the present invention.

Nucleotide sequence of IRES of FMDV
(SEQ ID NO. 29)
AGCAGGTTTCCCCAACTGACACAAAACGTGCAACTTGAAACTCCGCCTGG
TCTTTCCAGGTCTAGAGGGGTAACACTTTGTACTGCGTTTGGCTCCACGC
TCGATCCACTGGCGAGTGTTAGTAACAGCACTGTTGCTTCGTAGCGGAGC
ATGACGGCCGTGGGAACTCCTCCTTGGTAACAAGGACCCACGGGGCCAAA
AGCCACGCCCACACGGGCCCGTCATGTGTGCAACCCCAGCACGGCGACTT
TACTGCGAAACCCACTTTAAAGTGACATTGAAACTGGTACCCACACACTG
GTGACAGGCTAAGGATGCCCTTCAGGTACCCCGAGGTAACACGCGACACT
CGGGATCTGAGAAGGGGACTGGGGCTTCTATAAAAGCGCTCGGTTTAAAA
AGCTTCTATGCCTGAATAGGTGACCGGAGGTCGGCACCTTTCCTTTACAA
TTAAAGACCCT

The following nucleotide sequences according to SEQ ID Nos. 30 and 31 show examples of F2A peptides from FMDV that mediate cotranslational cleavage usable for the purposes of the present invention.

Nucleotide sequence of F2A peptide, version 1,
of FMDV
(SEQ ID NO. 30)
GTGAAGCAGACACTCAATTTCGACCTTCTGAAGTTGGCTGGAGATGTTGA
GTCTAACCCAGGCCCC
Nucleotide sequence of F2A peptide, version 2,
of FMDV
(SEQ ID NO. 31)
GTCAAACAGACCTTGAACTTCGACTTGCTCAAACTGGCCGGGGATGTGGA
GTCCAATCCTGGACCT

In a preferred embodiment, the mRNA sequence according to the invention does not comprise a reporter gene or a marker gene. Preferably, the mRNA sequence according to the invention does not encode, for instance, luciferase; green fluorescent protein (GFP) and its variants (such as eGFP, RFP or BFP); α-globin; hypoxanthine-guanine phosphoribosyltransferase (HGPRT); β-galactosidase; galactokinase; alkaline phosphatase; secreted embryonic alkaline phosphatase (SEAP) or a resistance gene (such as a resistance gene against neomycin, puromycin, hygromycin and zeocin). In a preferred embodiment, the mRNA sequence according to the invention does not encode luciferase. In another embodiment, the mRNA sequence according to the invention does not encode GFP or a variant thereof.

In a further preferred embodiment, the mRNA sequence according to the invention does not encode a protein (or a fragment of a protein) derived from a virus belonging to the family of Orthomyxoviridae. Preferably the mRNA sequence does not encode a protein that is derived from an influenza virus, more preferably an influenza A virus. Preferably, the mRNA sequence according to the invention does not encode an influenza A protein selected from the group consisting of hemagglutinin (HA), neuraminidase (NA), nucleoprotein (NP), M1, M2, NS1, NS2 (NEP: nuclear export protein), PA, PB1 (polymerase basic 1), PB1-F2 and PB2. In another preferred embodiment, the mRNA sequence according to the invention does not encode ovalbumin (OVA) or a fragment thereof. Preferably, the mRNA sequence according to the invention does not encode an influenza A protein or ovalbumin.

By a further embodiment, the inventive mRNA sequence preferably comprises at least one of the following structural elements: a 5′- and/or 3′-untranslated region element (UTR element), particularly a 5′-UTR element which comprises or consists of a nucleic acid sequence which is derived from the 5′-UTR of a TOP gene or from a fragment, homolog or a variant thereof, or a 5′- and/or 3′-UTR element which may be derivable from a gene that provides a stable mRNA or from a homolog, fragment or variant thereof; a histone-stem-loop structure, preferably a histone-stem-loop in its 3′ untranslated region; a 5′-CAP structure; a poly-A tail; or a poly(C) sequence.

In a further embodiment, there is provided a composition comprising a plurality of RNA molecules of the embodiments (e.g., encoding an Ebolavirus or Marburgvirus antigen) in pharmaceutically acceptable carrier, wherein at least about 90%, 91%, 92%, 93%, 94%, 95%, 96%, 97%, 98%, 99% or greater of the RNA in the composition comprises a poly(A) sequence that differs in length by no more than 10 nucleotides. In a preferred embodiment at least about 90%, 91%, 92%, 93%, 94%, 95%, 96%, 97%, 98%, 99% or greater of the RNA in the composition comprises a poly(A) sequence of identical length. In certain embodiments, the poly(A) sequence is positioned at the 3′ end of the RNA, with no other nucleotides positioned 3′ relative the poly(A) sequence. In still a further embodiment, there is provided a composition comprising a plurality of RNA molecules of the embodiments in pharmaceutically acceptable carrier, wherein said plurality of RNA molecules comprise both capped and uncapped RNAs. For example, in some aspects, a composition comprises a plurality of RNA molecules wherein no more than 95%, 90%, 80%, 70% or 60% of the RNAs comprise a cap and the remaining RNA molecules are uncapped.

In a preferred embodiment of the first aspect of the present invention the mRNA sequence comprises at least one 5′- or 3′-UTR element. In this context an UTR element comprises or consists of a nucleic acid sequence which is derived from the 5′- or 3′-UTR of any naturally occurring gene or which is derived from a fragment, a homolog or a variant of the 5′- or 3′-UTR of a gene. Preferably the 5′- or 3′-UTR element used according to the present invention is heterologous to the coding region of the inventive mRNA sequence. Even if 5′- or 3′-UTR elements derived from naturally occurring genes are preferred, also synthetically engineered UTR elements may be used in the context of the present invention.

In a particularly preferred embodiment of the first aspect of the present invention the mRNA sequence comprises at least one 5′-untranslated region element (5′-UTR element) which comprises or consists of a nucleic acid sequence which is derived from the 5′-UTR of a TOP gene or which is derived from a fragment, homolog or variant of the 5′-UTR of a TOP gene.

It is particularly preferred that the 5′-UTR element does not comprise a TOP-motif or a 5′TOP, as defined above.

In some embodiments, the nucleic acid sequence of the 5′-UTR element which is derived from a 5′-UTR of a TOP gene terminates at its 3′-end with a nucleotide located at position 1, 2, 3, 4, 5, 6, 7, 8, 9 or 10 upstream of the start codon (e.g. A(U/T)G) of the gene or mRNA it is derived from. Thus, the 5′-UTR element does not comprise any part of the protein coding region. Thus, preferably, the only protein coding part of the inventive mRNA sequence is provided by the coding region.

The nucleic acid sequence which is derived from the 5′-UTR of a TOP gene is preferably derived from a eukaryotic TOP gene, preferably a plant or animal TOP gene, more preferably a chordate TOP gene, even more preferably a vertebrate TOP gene, most preferably a mammalian TOP gene, such as a human TOP gene.

For example, the 5′-UTR element is preferably selected from 5′-UTR elements comprising or consisting of a nucleic acid sequence which is derived from a nucleic acid sequence selected from the group consisting of SEQ ID Nos. 1-1363, SEQ ID NO. 1395, SEQ ID NO. 1421 and SEQ ID NO. 1422 of the patent application WO2013/143700, whose disclosure is incorporated herein by reference, from the homologs of SEQ ID Nos. 1-1363, SEQ ID NO. 1395, SEQ ID NO. 1421 and SEQ ID NO. 1422 of the patent application WO2013/143700, from a variant thereof, or preferably from a corresponding RNA sequence. The term “homologs of SEQ ID Nos. 1-1363, SEQ ID NO. 1395, SEQ ID NO. 1421 and SEQ ID NO. 1422 of the patent application WO2013/143700” refers to sequences of other species than Homo sapiens, which are homologous to the sequences according to SEQ ID Nos. 1-1363, SEQ ID NO. 1395, SEQ ID NO. 1421 and SEQ ID NO. 1422 of the patent application WO2013/143700.

In a preferred embodiment, the 5′-UTR element comprises or consists of a nucleic acid sequence which is derived from a nucleic acid sequence extending from nucleotide position 5 (i.e. the nucleotide that is located at position 5 in the sequence) to the nucleotide position immediately 5′ to the start codon (located at the 3′ end of the sequences), e.g. the nucleotide position immediately 5′ to the ATG sequence, of a nucleic acid sequence selected from SEQ ID Nos. 1-1363, SEQ ID NO. 1395, SEQ ID NO. 1421 and SEQ ID NO. 1422 of the patent application WO2013/143700, from the homologs of SEQ ID Nos. 1-1363, SEQ ID NO. 1395, SEQ ID NO. 1421 and SEQ ID NO. 1422 of the patent application WO2013/143700 from a variant thereof, or a corresponding RNA sequence. It is particularly preferred that the 5′ UTR element is derived from a nucleic acid sequence extending from the nucleotide position immediately 3′ to the 5′TOP to the nucleotide position immediately 5′ to the start codon (located at the 3′ end of the sequences), e.g. the nucleotide position immediately 5′ to the ATG sequence, of a nucleic acid sequence selected from SEQ ID Nos. 1-1363, SEQ ID NO. 1395, SEQ ID NO. 1421 and SEQ ID NO. 1422 of the patent application WO2013/143700, from the homologs of SEQ ID Nos. 1-1363, SEQ ID NO. 1395, SEQ ID NO. 1421 and SEQ ID NO. 1422 of the patent application WO2013/143700, from a variant thereof, or a corresponding RNA sequence.

In a particularly preferred embodiment, the 5′-UTR element comprises or consists of a nucleic acid sequence which is derived from a 5′-UTR of a TOP gene encoding a ribosomal protein or from a variant of a 5′-UTR of a TOP gene encoding a ribosomal protein. For example, the 5′-UTR element comprises or consists of a nucleic acid sequence which is derived from a 5′-UTR of a nucleic acid sequence according to any of SEQ ID NOs: 67, 170, 193, 244, 259, 554, 650, 675, 700, 721, 913, 1016, 1063, 1120, 1138, and 1284-1360 of the patent application WO2013/143700, a corresponding RNA sequence, a homolog thereof, or a variant thereof as described herein, preferably lacking the 5′TOP motif. As described above, the sequence extending from position 5 to the nucleotide immediately 5′ to the ATG (which is located at the 3′end of the sequences) corresponds to the 5′-UTR of said sequences.

Preferably, the 5′-UTR element comprises or consists of a nucleic acid sequence which is derived from a 5′-UTR of a TOP gene encoding a ribosomal Large protein (RPL) or from a homolog or variant of a 5′-UTR of a TOP gene encoding a ribosomal Large protein (RPL). For example, the 5′-UTR element comprises or consists of a nucleic acid sequence which is derived from a 5′-UTR of a nucleic acid sequence according to any of SEQ ID NOs: 67, 259, 1284-1318, 1344, 1346, 1348-1354, 1357, 1358, 1421 and 1422 of the patent application WO2013/143700, a corresponding RNA sequence, a homolog thereof, or a variant thereof as described herein, preferably lacking the 5′TOP motif.

In a particularly preferred embodiment, the 5′-UTR element comprises or consists of a nucleic acid sequence which is derived from the 5′-UTR of a ribosomal protein Large 32 gene, preferably from a vertebrate ribosomal protein Large 32 (L32) gene, more preferably from a mammalian ribosomal protein Large 32 (L32) gene, most preferably from a human ribosomal protein Large 32 (L32) gene, or from a variant of the 5′-UTR of a ribosomal protein Large 32 gene, preferably from a vertebrate ribosomal protein Large 32 (L32) gene, more preferably from a mammalian ribosomal protein Large 32 (L32) gene, most preferably from a human ribosomal protein Large 32 (L32) gene, wherein preferably the 5′-UTR element does not comprise the 5′TOP of said gene.

A preferred sequence for a 5′-UTR element corresponds to SEQ ID NO. 1368 of the patent application WO2013/143700 and reads as follows:

Nucleotide sequence for 5′-UTR element

(SEQ ID NO. 32)

GGCGCTGCCTACGGAGGTGGCAGCCATCTCCTTCTCGGCATC

Accordingly, in a particularly preferred embodiment, the 5′-UTR element comprises or consists of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO. 1368 of the patent application WO2013/143700 (5′-UTR of human ribosomal protein Large 32 lacking the 5′ terminal oligopyrimidine tract, SEQ ID NO. 32) or preferably to a corresponding RNA sequence, or wherein the at least one 5′-UTR element comprises or consists of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO. 31 or more preferably to a corresponding RNA sequence, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

In some embodiments, the inventive mRNA sequence comprises a 5′-UTR element which comprises or consists of a nucleic acid sequence which is derived from the 5′-UTR of a vertebrate TOP gene, such as a mammalian, e.g. a human TOP gene, selected from RPSA, RPS2, RPS3, RPS3A, RPS4, RPS5, RPS6, RPS7, RPS8, RPS9, RPS10, RPS11, RPS12, RPS13, RPS14, RPS15, RPS15A, RPS16, RPS17, RPS18, RPS19, RPS20, RPS21, RPS23, RPS24, RPS25, RPS26, RPS27, RPS27A, RPS28, RPS29, RPS30, RPL3, RPL4, RPL5, RPL6, RPL7, RPL7A, RPL8, RPL9, RPL10, RPL10A, RPL11, RPL12, RPL13, RPL13A, RPL14, RPL15, RPL17, RPL18, RPL18A, RPL19, RPL21, RPL22, RPL23, RPL23A, RPL24, RPL26, RPL27, RPL27A, RPL28, RPL29, RPL30, RPL31, RPL32, RPL34, RPL35, RPL35A, RPL36, RPL36A, RPL37, RPL37A, RPL38, RPL39, RPL40, RPL41, RPLP0, RPLP1, RPLP2, RPLP3, RPLP0, RPLP1, RPLP2, EEF1A1, EEF1B2, EEF1D, EEF1G, EEF2, EIF3E, EIF3F, EIF3H, EIF2S3, EIF3C, EIF3K, EIF3EIP, EIF4A2, PABPC1, HNRNPA1, TPT1, TUBB1, UBA52, NPM1, ATP5G2, GNB2L1, NME2, UQCRB, or from a homolog or variant thereof, wherein preferably the 5′-UTR element does not comprise a TOP-motif or the 5′TOP of said genes, and wherein optionally the 5′-UTR element starts at its 5′-end with a nucleotide located at position 1, 2, 3, 4, 5, 6, 7, 8, 9 or 10 downstream of the 5′terminal oligopyrimidine tract (TOP) and wherein further optionally the 5′-UTR element which is derived from a 5′-UTR of a TOP gene terminates at its 3′-end with a nucleotide located at position 1, 2, 3, 4, 5, 6, 7, 8, 9 or 10 upstream of the start codon (A(U/T)G) of the gene it is derived from.

In further particularly preferred embodiments, the 5′-UTR element comprises or consists of a nucleic acid sequence which is derived from the 5′-UTR of a ribosomal protein Large 32 gene (RPL32), a ribosomal protein Large 35 gene (RPL35), a ribosomal protein Large 21 gene (RPL21), an ATP synthase, H+ transporting, mitochondrial F1 complex, alpha subunit 1, cardiac muscle (ATP5A1) gene, an hydroxysteroid (17-beta) dehydrogenase 4 gene (HSD17B4), an androgen-induced 1 gene (AIG1), cytochrome c oxidase subunit Vlc gene (COX6C), or a N-acylsphingosine amidohydrolase (acid ceramidase) 1 gene (ASAH1) or from a variant thereof, preferably from a vertebrate ribosomal protein Large 32 gene (RPL32), a vertebrate ribosomal protein Large 35 gene (RPL35), a vertebrate ribosomal protein Large 21 gene (RPL21), a vertebrate ATP synthase, H+ transporting, mitochondrial F1 complex, alpha subunit 1, cardiac muscle (ATP5A1) gene, a vertebrate hydroxysteroid (17-beta) dehydrogenase 4 gene (HSD17B4), a vertebrate androgen-induced 1 gene (AIG1), a vertebrate cytochrome c oxidase subunit Vlc gene (COX6C), or a vertebrate N-acylsphingosine amidohydrolase (acid ceramidase) 1 gene (ASAH1) or from a variant thereof, more preferably from a mammalian ribosomal protein Large 32 gene (RPL32), a ribosomal protein Large 35 gene (RPL35), a ribosomal protein Large 21 gene (RPL21), a mammalian ATP synthase, H+ transporting, mitochondrial F1 complex, alpha subunit 1, cardiac muscle (ATP5A1) gene, a mammalian hydroxysteroid (17-beta) dehydrogenase 4 gene (HSD17B4), a mammalian androgen-induced 1 gene (AIG1), a mammalian cytochrome c oxidase subunit Vlc gene (COX6C), or a mammalian N-acylsphingosine amidohydrolase (acid ceramidase) 1 gene (ASAH1) or from a variant thereof, most preferably from a human ribosomal protein Large 32 gene (RPL32), a human ribosomal protein Large 35 gene (RPL35), a human ribosomal protein Large 21 gene (RPL21), a human ATP synthase, H+ transporting, mitochondrial F1 complex, alpha subunit 1, cardiac muscle (ATP5A1) gene, a human hydroxysteroid (17-beta) dehydrogenase 4 gene (HSD17B4), a human androgen-induced 1 gene (AIG1), a human cytochrome c oxidase subunit Vlc gene (COX6C), or a human N-acylsphingosine amidohydrolase (acid ceramidase) 1 gene (ASAH1) or from a variant thereof, wherein preferably the 5′-UTR element does not comprise the 5′TOP of said gene.

Accordingly, in a particularly preferred embodiment, the 5′-UTR element comprises or consists of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO. 1368, or SEQ ID NOs 1412-1420 of the patent application WO2013/143700, or a corresponding RNA sequence, or wherein the at least one 5′-UTR element comprises or consists of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO. 1368, or SEQ ID NOs 1412-1420 of the patent application WO2013/143700, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

Accordingly, in a particularly preferred embodiment, the 5′-UTR element comprises or consists of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according SEQ ID NO. 1414 of the patent application WO2013/143700 (5′-UTR of ATP5A1 lacking the 5′ terminal oligopyrimidine tract) or preferably to a corresponding RNA sequence, or wherein the at least one 5′-UTR element comprises or consists of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO. 1414 of the patent application WO2013/143700 or more preferably to a corresponding RNA sequence, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

In a further preferred embodiment, the inventive mRNA sequence further comprises at least one 3′-UTR element which comprises or consists of a nucleic acid sequence derived from the 3′-UTR of a chordate gene, preferably a vertebrate gene, more preferably a mammalian gene, most preferably a human gene, or from a variant of the 3′-UTR of a chordate gene, preferably a vertebrate gene, more preferably a mammalian gene, most preferably a human gene.

The term ‘3′-UTR element’ refers to a nucleic acid sequence which comprises or consists of a nucleic acid sequence that is derived from a 3′-UTR or from a variant of a 3′-UTR. A 3′-UTR element in the sense of the present invention may represent the 3′-UTR of an mRNA. Thus, in the sense of the present invention, preferably, a 3′-UTR element may be the 3′-UTR of an mRNA, preferably of an artificial mRNA, or it may be the transcription template for a 3′-UTR of an mRNA. Thus, a 3′-UTR element preferably is a nucleic acid sequence which corresponds to the 3′-UTR of an mRNA, preferably to the 3′-UTR of an artificial mRNA, such as an mRNA obtained by transcription of a genetically engineered vector construct. Preferably, the 3′-UTR element fulfils the function of a 3′-UTR or encodes a sequence which fulfils the function of a 3′-UTR.

Preferably, the inventive mRNA sequence comprises a 3′-UTR element which may be derivable from a gene that relates to an mRNA with an enhanced half-life (that provides a stable mRNA), for example a 3′-UTR element as defined and described below.

In a particularly preferred embodiment, the 3′-UTR element comprises or consists of a nucleic acid sequence which is derived from a 3′-UTR of a gene selected from the group consisting of an albumin gene, an α-globin gene, a β-globin gene, a tyrosine hydroxylase gene, a lipoxygenase gene, and a collagen alpha gene, such as a collagen alpha 1 (I) gene, or from a variant of a 3′-UTR of a gene selected from the group consisting of an albumin gene, an α-globin gene, a β-globin gene, a tyrosine hydroxylase gene, a lipoxygenase gene, and a collagen alpha gene, such as a collagen alpha 1(I) gene according to SEQ ID NO. 1369-1390 of the patent application WO2013/143700 whose disclosure is incorporated herein by reference. In a particularly preferred embodiment, the 3′-UTR element comprises or consists of a nucleic acid sequence which is derived from a 3′-UTR of an albumin gene, preferably a vertebrate albumin gene, more preferably a mammalian albumin gene, most preferably a human albumin gene according SEQ ID No: 1369 of the patent application WO2013/143700. The mRNA sequence may comprise or consist of a nucleic acid sequence which is derived from the 3′-UTR of the human albumin gene according to GenBank Accession number NM_000477.5, or from a fragment or variant thereof.

In this context it is particularly preferred that the inventive mRNA sequence comprises a 3′-UTR element comprising a corresponding RNA sequence derived from the nucleic acid sequences according to SEQ ID NO. 1369-1390 of the patent application WO2013/143700 or a fragment, homolog or variant thereof.

Most preferably the 3′-UTR element comprises the nucleic acid sequence derived from a fragment of the human albumin gene according to SEQ ID No: 1376 of the patent application WO2013/143700, in the following referred to as SEQ ID NO. 33.

Nucleotide sequence of 3′-UTR element of human
albumin gene
(SEQ ID NO. 33)
CATCACATTTAAAAGCATCTCAGCCTACCATGAGAATAAGAGAAAGAAAA
TGAAGATCAATAGCTTATTCATCTCTTTTTCTTTTTCGTTGGTGTAAAGC
CAACACCCTGTCTAAAAAACATAAATTTCTTTAATCATTTTGCCTCTTTT
CTCTGTGCTTCAATTAATAAAAAATGGAAAGAACCT

In another particularly preferred embodiment, the 3′-UTR element comprises or consists of a nucleic acid sequence which is derived from a 3′-UTR of an α-globin gene, preferably a vertebrate α- or β-globin gene, more preferably a mammalian α- or β-globin gene, most preferably a human α- or β-globin gene according to SEQ ID NO. 1370 of the patent application WO2013/143700 (3′-UTR of Homo sapiens hemoglobin, alpha 1 (HBA1)), or according to SEQ ID NO. 1371 of the patent application WO2013/143700 (3′-UTR of Homo sapiens hemoglobin, alpha 2 (HBA2)), or according to SEQ ID NO. 1372 of the patent application WO2013/143700 (3′-UTR of Homo sapiens hemoglobin, beta (HBB)).

For example, the 3′-UTR element may comprise or consist of the center, α-complex-binding portion of the 3′-UTR of an α-globin gene, such as of a human «-globin gene, preferably according to SEQ ID NO. 34 (corresponding to SEQ ID NO. 1393 of the patent application WO2013/143700).

Nucleotide sequence of 3′ UTR element of an
α-globin gene

(SEQ ID NO. 34)

GCCCGATGGGCCTCCCAACGGGCCCTCCTCCCCTCCTTGCACCG

In this context it is particularly preferred that the 3′-UTR element of the inventive mRNA comprises or consists of a corresponding RNA sequence of the nucleic acid sequence according to the above or a homolog, a fragment or variant thereof.

The term ‘a nucleic acid sequence which is derived from the 3′-UTR of a [ . . . ] gene’ preferably refers to a nucleic acid sequence which is based on the 3′-UTR sequence of a [ . . . ] gene or on a part thereof, such as on the 3′-UTR of an albumin gene, an α-globin gene, a β-globin gene, a tyrosine hydroxylase gene, a lipoxygenase gene, or a collagen alpha gene, such as a collagen alpha 1(I) gene, preferably of an albumin gene or on a part thereof. This term includes sequences corresponding to the entire 3′-UTR sequence, i.e. the full length 3′-UTR sequence of a gene, and sequences corresponding to a fragment of the 3′-UTR sequence of a gene, such as an albumin gene, α-globin gene, β-globin gene, tyrosine hydroxylase gene, lipoxygenase gene, or collagen alpha gene, such as a collagen alpha 1(I) gene, preferably of an albumin gene.

The term ‘a nucleic acid sequence which is derived from a variant of the 3′-UTR of a [ . . . ] gene’ preferably refers to a nucleic acid sequence which is based on a variant of the 3′-UTR sequence of a gene, such as on a variant of the 3′-UTR of an albumin gene, an α-globin gene, a β-globin gene, a tyrosine hydroxylase gene, a lipoxygenase gene, or a collagen alpha gene, such as a collagen alpha 1(I) gene, or on a part thereof as described above. This term includes sequences corresponding to the entire sequence of the variant of the 3′-UTR of a gene, i.e. the full length variant 3′-UTR sequence of a gene, and sequences corresponding to a fragment of the variant 3′-UTR sequence of a gene. A fragment in this context preferably consists of a continuous stretch of nucleotides corresponding to a continuous stretch of nucleotides in the full-length variant 3′-UTR, which represents at least 20%, preferably at least 30%, more preferably at least 40%, more preferably at least 50%, even more preferably at least 60%, even more preferably at least 70%, even more preferably at least 80%, and most preferably at least 90% of the full-length variant 3′-UTR. Such a fragment of a variant, in the sense of the present invention, is preferably a functional fragment of a variant as described herein.

Preferably, the at least one 5′-UTR element and the at least one 3′-UTR element act synergistically to increase protein production from the inventive mRNA sequence as described above.

In a particularly preferred embodiment, the inventive mRNA sequence comprising a coding region, encoding at least one antigenic peptide or protein of a virus of Ebolavirus or Marburvirus or a fragment, variant or derivative thereof according to the above, comprises a histone stem-loop sequence/structure. Such histone stem-loop sequences are preferably selected from histone stem-loop sequences as disclosed in WO 2012/019780, whose disclosure is incorporated herewith by reference.

A histone stem-loop sequence, suitable to be used within the present invention, is preferably selected from at least one of the following formulae (I) or (II):

(Stem-Loop Sequence without Stem Bordering Elements):

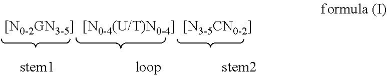

(Stem-Loop Sequence with Stem Bordering Elements):

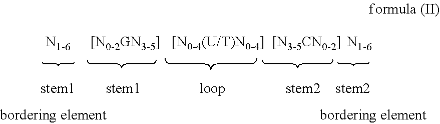

wherein:

| | |
|---|---|
| stem1 or stem2 | is a consecutive sequence of 1 to 6, preferably |
| bordering | of 2 to 6, more preferably of 2 to 5, even more |
| elements N₁₋₆ | preferably of 3 to 5, most preferably of 4 to 5 or |
| | 5 N, wherein each N is independently from |
| | another selected from a nucleotide selected from |
| | A, U, T, G and C, or a nucleotide analogue |
| | thereof; |
| stem1 [N₀₋₂GN₃₋₅] | is reverse complementary or partially reverse |
| | complementary with element stem2, and is a |
| | consecutive sequence between of 5 to 7 |
| | nucleotides; |
| | wherein N₀₋₂ is a consecutive sequence of 0 to |
| | 2, preferably of 0 to 1, more preferably of 1 N, |
| | wherein each N is independently from another |
| | selected from a nucleotide selected from A, U, |
| | T, G and C or a nucleotide analogue thereof; |
| | wherein N₃₋₅ is a consecutive sequence of 3 to |
| | 5, preferably of 4 to 5, more preferably of 4 N, |
| | wherein each N is independently from another |
| | selected from a nucleotide selected from A, U, |
| | T, G and C or a nucleotide analogue thereof, and |
| | wherein G is guanosine or an analogue thereof, |
| | and may be optionally replaced by a cytidine or |
| | an analogue thereof, provided that its |
| | complementary nucleotide cytidine in stem2 is |
| | replaced by guanosine; |
| loop sequence | is located between elements stem1 and stem2, |
| [N₀₋₄(U/T)N₀₋₄] | and is a consecutive sequence of 3 to 5 |
| | nucleotides, more preferably of 4 nucleotides; |
| | wherein each No-4 is independent from another |
| | a consecutive sequence of 0 to 4, preferably of |
| | 1 to 3, more preferably of 1 to 2 N, wherein each |
| | N is independently from another selected from a |
| | nucleotide selected from A, U, T, G and C or a |
| | nucleotide analogue thereof; and |
| | wherein U/T represents uridine, or optionally |
| | thymidine; |
| stem2 [N₃₋₅CN₀₋₂] | is reverse complementary or partially reverse |
| | complementary with element stem1, and is a |
| | consecutive sequence between of 5 to 7 |
| | nucleotides; |
| | wherein N₃₋₅ is a consecutive sequence of 3 to |
| | 5, preferably of 4 to 5, more preferably of 4 N, |
| | wherein each N is independently from another |
| | selected from a nucleotide selected from A, U, |
| | T, G and C or a nucleotide analogue thereof; |
| | wherein N₀₋₂ is a consecutive sequence of 0 to |
| | 2, preferably of 0 to 1, more preferably of 1 N, |
| | wherein each N is independently from another |
| | selected from a nucleotide selected from A, U, |
| | T, G or C or a nucleotide analogue thereof; and |
| | wherein C is cytidine or an analogue thereof, |
| | and may be optionally replaced by a guanosine |
| | or an analogue thereof provided that its |
| | complementary nucleoside guanosine in stem1 |
| | is replaced by cytidine; |

wherein
stem1 and stem2 are capable of base pairing with each other forming a reverse complementary sequence, wherein base pairing may occur between stem1 and stem2, e.g. by Watson-Crick base pairing of nucleotides A and U/T or G and C or by non-Watson-Crick base pairing e.g. wobble base pairing, reverse Watson-Crick base pairing, Hoogsteen base pairing, reverse Hoogsteen base pairing or are capable of base pairing with each other forming a partially reverse complementary sequence, wherein an incomplete base pairing may occur between stem1 and stem2, on the basis that one ore more bases in one stem do not have a complementary base in the reverse complementary sequence of the other stem.

According to a further preferred embodiment of the first inventive aspect, the inventive mRNA sequence may comprise at least one histone stem-loop sequence according to at least one of the following specific formulae (Ia) or (IIa):

(Stem-Loop Sequence without Stem Bordering Elements):

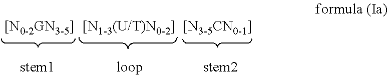

(Stem-Loop Sequence with Stem Bordering Elements):

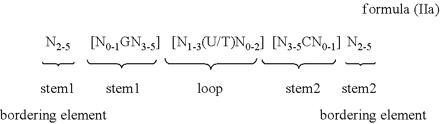

wherein:
N, C, G, T and U are as defined above.

According to a further more particularly preferred embodiment of the first aspect, the inventive mRNA sequence may comprise at least one histone stem-loop sequence according to at least one of the following specific formulae (Ib) or (IIb):

(Stem-Loop Sequence without Stem Bordering Elements):

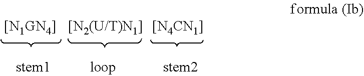

(Stem-Loop Sequence with Stem Bordering Elements):

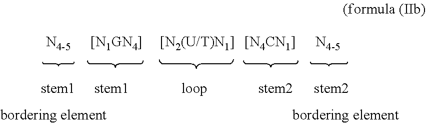

wherein:
N, C, G, T and U are as defined above.

A particular preferred histone stem-loop sequence is the nucleic acid sequence according to SEQ ID NO. 35.

Histone stem-loop nucleotide sequence

(SEQ ID NO. 35)

CAAAGGCTCTTTTCAGAGCCACCA

More preferably the stem-loop sequence is the corresponding RNA sequence of the nucleic acid sequence according to SEQ ID NO. 36

Histone stem-loop RNA sequence

(SEQ ID NO. 36)

CAAAGGCUCUUUUCAGAGCCACCA

In a particularly preferred embodiment of the first aspect of the present invention, the inventive mRNA sequence comprises additionally to the coding region encoding at least one antigenic peptide or protein of Ebolavirus or Marburgvirus as outlined above or a fragment, variant or derivative thereof, a poly(A) sequence, also called poly-A-tail, preferably at the 3′-terminus of the inventive mRNA sequence. When present, such a poly(A) sequence comprises a sequence of about 25 to about 400 adenosine nucleotides, preferably a sequence of about 50 to about 400 adenosine nucleotides, more preferably a sequence of about 50 to about 300 adenosine nucleotides, even more preferably a sequence of about 50 to about 250 adenosine nucleotides, most preferably a sequence of about 60 to about 250 adenosine nucleotides. In this context the term “about” refers to a deviation of +10% of the value(s) it is attached to. This poly(A) sequence is preferably located 3′ of the coding region comprised in the inventive mRNA sequence according to the first aspect of the present invention.

According to a further preferred embodiment the inventive mRNA sequence can be modified by a sequence of at least 10 cytosines, preferably at least 20 cytosines, more preferably at least 30 cytosines (so-called “poly(C) sequence”). Particularly, the inventive mRNA sequence may contain a poly(C) sequence of typically about 10 to 200 cytosine nucleotides, preferably about 10 to 100 cytosine nucleotides, more preferably about 10 to 70 cytosine nucleotides or even more preferably about 20 to 50 or even 20 to 30 cytosine nucleotides. This poly(C) sequence is preferably located 3′ of the coding region, more preferably 3′ of an optional poly(A) sequence comprised in the inventive mRNA sequence according to the first aspect of the present invention.

In this context the inventive mRNA sequence may comprise in a specific embodiment:
a.) a 5′-CAP structure, preferably m7GpppN;
b.) a coding region encoding at least one antigenic peptide or protein of a virus of the genus Ebolavirus or Marburgvirus, wherein the peptide or protein is derived from the glycoprotein (GP) and/or the matrix protein 40 (VP40) and/or the nucleoprotein (NP) or a virus of the genus Ebolavirus or Marburgvirus;
c.) a poly(A) sequence preferably comprising 64 adenosines; and
d.) optionally, a poly(C) sequence, preferably comprising 30 cytosines.

In a particularly preferred embodiment of the first aspect of the present invention the inventive mRNA sequence comprising a coding region encoding at least one antigenic peptide or protein of Ebolavirus or Marburgvirus or a fragment, variant or derivative thereof, comprises preferably in 5′- to 3′-direction:
a.) a 5′-CAP structure, preferably m7GpppN;
b.) a coding region encoding at least one antigenic peptide or protein of a virus of the genus Ebolavirus or Marburgvirus, wherein the peptide or protein is derived from the glycoprotein (GP) and/or the matrix protein 40 (VP40) and/or the nucleoprotein (NP) or a virus of the genus Ebolavirus or Marburgvirus;
c.) a poly(A) sequence preferably comprising 64 adenosines;
d.) optionally, a poly(C) sequence, preferably comprising 30 cytosines; and
e.) a histone-stem-loop, preferably comprising the corresponding RNA sequence of the nucleic acid sequence according to SEQ ID NO. 35.

In a further particularly preferred embodiment of the first aspect of the present invention the inventive mRNA sequence comprising a coding region encoding at least one antigenic peptide or protein of Ebolavirus or Marburgvirus or a fragment, variant or derivative thereof, comprises preferably in 5′- to 3′-direction:
a.) a 5′-CAP structure, preferably m7GpppN;
b.) a coding region encoding at least one antigenic peptide or protein of a virus of the genus Ebolavirus or Marburgvirus, wherein the peptide or protein is derived from the glycoprotein (GP) and/or the matrix protein 40 (VP40) and/or the nucleoprotein (NP) or a virus of the genus Ebolavirus or Marburgvirus;
c.) optionally, a 3′-UTR element derived from an alpha globin gene, preferably comprising the corresponding RNA sequence of the nucleic acid sequence according to SEQ ID NO. 34, a homolog, a fragment, or a variant thereof;
d.) a poly(A) sequence preferably comprising 64 adenosines;
e.) optionally, a poly(C) sequence, preferably comprising 30 cytosines; and
f.) a histone-stem-loop, preferably comprising the corresponding RNA sequence of the nucleic acid sequence according to SEQ ID NO. 35.

In another particular preferred embodiment the inventive mRNA sequence encoding at least one antigenic peptide or protein of Ebolavirus or Marburgvirus or a fragment, variant or derivative thereof, comprises preferably in 5′- to 3′-direction:
a.) a 5′-CAP structure, preferably m7GpppN;
b.) optionally, a 5′-UTR element derived from a TOP gene, preferably derived from the corresponding RNA sequence of the nucleic acid sequence according to SEQ ID NO. 32, a homolog, a fragment, or a variant thereof;
c.) a coding region encoding at least one antigenic peptide or protein of a virus of the genus Ebolavirus or Marburgvirus, wherein the peptide or protein is derived from the glycoprotein (GP) and/or the matrix protein 40 (VP40) and/or the nucleoprotein (NP) or a virus of the genus Ebolavirus or Marburgvirus;
d.) optionally, a 3′-UTR element derived of a gene providing a stable mRNA, preferably derived from the corresponding RNA sequence of a nucleic acid sequence according to SEQ ID NO. 33, a homolog, a fragment, or a variant thereof;
e.) a poly(A) sequence preferably comprising 64 adenosines;
f.) optionally, a poly(C) sequence, preferably comprising 30 cytosines; and
g.) a histone-stem-loop, preferably comprising the corresponding RNA sequence of the nucleic acid sequence according to SEQ ID NO. 35.

The coding region might encode at least partially one of the amino acid sequences according to SEQ ID Nos. 1-18 or fragments, variants or derivatives thereof. Furthermore the coding region of the inventive mRNA sequence may encode a combination of at least two of these amino acid sequences or a combination of fragments, variants or derivatives thereof.

Additionally the coding region might be or might comprise at least partially one of the sequences according to SEQ ID Nos 20-27, preferably the corresponding RNA sequences, or fragments, homologs or variants thereof. Furthermore, the mRNA might comprise a combination of at least two of these sequences preferably the corresponding RNA sequences, or a combination of fragments, homologs or variants thereof.

As outlined above in an especially preferred embodiment of the invention the mRNA sequences are optimised for the purposes of the invention, wherein the G/C content of the coding region is increased compared with the G/C content of the coding region of the wild type mRNA. In this context the modified wild type nucleotide sequence which include the modified editing site of a stretch of eight adenosine nucleotides as defined above is to be understood as wild type mRNA respectively as basis for the optimisation.

For further improvement of the resistance to e.g. in vivo degradation (e.g. by an exo- or endo-nuclease), the inventive mRNA sequence is provided as a stabilized nucleic acid, e.g. in the form of a modified nucleic acid. In this context the G/C content is preferably increased as outlined above. According to a further embodiment of the invention it is therefore preferred that the inventive mRNA sequence is further stabilized, preferably by backbone modifications, sugar modifications and/or base modifications. All of these modifications may be introduced into the inventive mRNA sequence without impairing the mRNA's function to be translated into the antigenic function derived from the Ebolavirus or Marburgvirus peptide or protein.

A backbone modification in the context of the present invention is preferably a modification in which phosphates of the backbone of the nucleotides contained in the inventive mRNA sequence are chemically modified, e.g. anionic internucleoside linkage, N3′→P5′ modifications, replacement of non-bridging oxygen atoms by boranes, neutral internucleoside linkage, amide linkage of the nucleosides, methylene(methylimino) linkages, formacetal and thioformacetal linkages, introduction of sulfonyl groups, or the like.

A sugar modification in the context of the present invention is preferably a chemical modification of the sugar of the nucleotides of the inventive mRNA sequence, e.g. methylation of the ribose residue or the like.

A base modification in the context of the present invention is preferably a chemical modification of the base moiety of the nucleotides of the inventive RNA sequence. In this context, nucleotide analogues or modifications are preferably selected from nucleotide analogues, which are applicable for transcription and/or translation.

### Sugar Modifications:

The modified nucleosides and nucleotides, which may be incorporated into a modified mRNA as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R=H, alkyl, cycloalkyl, aryl, aralkyl, heteroaryl or sugar); polyethyleneglycols (PEG), —O(CH₂CH₂O)nCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O.

The sugar group can also contain one or more carbons that possess the opposite stereochemical configuration than that of the corresponding carbon in ribose. Thus, a modified mRNA can include nucleotides containing, for instance, arabinose as the sugar.

### Backbone Modifications:

The phosphate backbone may further be modified in the modified nucleosides and nucleotides, which may be incorporated into a modified mRNA as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

### Base Modifications:

The modified nucleosides and nucleotides, which may be incorporated into a modified mRNA as described herein can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particularly preferred embodiments of the present invention, the nucleotide analogues/modifications are selected from base modifications, which are preferably selected from 2-amino-6-chloropurineriboside-5′-triphosphate, 2-aminopurine-riboside-5′-triphosphate; 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate.

In some embodiments, modified nucleosides include pyridine-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyluridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-I-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, I-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group.

In specific embodiments, a modified nucleoside is 5′-O-(1-thiophosphate)-adenosine, 5′-O-(1-thiophosphate)-cytidine, 5′-O-(1-thiophosphate)-guanosine, 5′-O-(1-thiophosphate)-uridine or 5′-O-(1-thiophosphate)-pseudouridine.

In further specific embodiments, a modified RNA may comprise nucleoside modifications selected from 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, pyrrolo-cytidine, inosine, α-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, α-thio-adenosine, 8-azido-adenosine, 7-deaza-adenosine. Further nucleotide analogues are such as those disclosed in WO2013/052523.

### Lipid Modification:

According to a further embodiment, a modified mRNA as defined herein can contain a lipid modification. Such a lipid-modified mRNA typically comprises an mRNA as defined herein. Such a lipid-modified mRNA as defined herein typically further comprises at least one linker covalently linked with that mRNA, and at least one lipid covalently linked with the respective linker. Alternatively, the lipid-modified mRNA comprises at least one mRNA as defined herein and at least one (bifunctional) lipid covalently linked (without a linker) with that mRNA. According to a third alternative, the lipid-modified mRNA comprises an mRNA as defined herein, at least one linker covalently linked with that mRNA, and at least one lipid covalently linked with the respective linker, and also at least one (bifunctional) lipid covalently linked (without a linker) with that mRNA. In this context, it is particularly preferred that the lipid modification is present at the terminal ends of a linear mRNA sequence.

Modification of the 5′-End of a Modified mRNA:

According to another preferred embodiment of the invention, a modified mRNA as defined herein, can be modified by the addition of a so-called “5′ CAP” structure.

A 5′-cap is an entity, typically a modified nucleotide entity, which generally “caps” the 5′-end of a mature mRNA. A 5′-cap may typically be formed by a modified nucleotide, particularly by a derivative of a guanine nucleotide. Preferably, the 5′-cap is linked to the 5′-terminus via a 5′-5′-triphosphate linkage. A 5′-cap may be methylated, e.g. m7GpppN, wherein N is the terminal 5′ nucleotide of the nucleic acid carrying the 5′-cap, typically the 5′-end of an RNA. m7GpppN is the 5′-CAP structure which naturally occurs in mRNA transcribed by polymerase II and is therefore not considered as modification comprised in a modified RNA in this context. Accordingly, a modified RNA of the present invention may comprise a m7GpppN as 5′-CAP, but additionally the modified RNA comprises at least one further modification as defined herein.

Further examples of 5′cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. These modified 5′-CAP structures are regarded as at least one modification in this context.

Particularly preferred modified 5′-CAP structures are CAP1 (methylation of the ribose of the adjacent nucleotide of m7G), CAP2 (methylation of the ribose of the 2nd nucleotide downstream of the m7G), CAP3 (methylation of the ribose of the 3rd nucleotide downstream of the m7G), CAP4 (methylation of the ribose of the 4th nucleotide downstream of the m7G), ARCA (anti-reverse CAP analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

According to a further preferred embodiment of the invention, the inventive mRNA sequence is optimized for translation, preferably optimized for translation by replacing codons for less frequent tRNAs of a given amino acid by codons for more frequently occurring tRNAs of the respective amino acid. This is based on the finding that the translation efficiency is also determined by a different frequency in the occurrence of tRNAs in cells. Thus, if so-called “less frequent codons” are present in the inventive mRNA sequence to an increased extent, the corresponding modified RNA sequence is translated to a significantly poorer degree than in the case where codons coding for more frequent tRNAs are present. Preferably, the coding region of the inventive mRNA sequence is modified compared to the corresponding region of the wild type mRNA sequence or coding sequence such that at least one codon of the wild type sequence which codes for a tRNA which is relatively rare or less frequent in the cell is exchanged for a codon which codes for a tRNA which is more or most frequent in the cell and carries the same amino acid as the relatively rare or less frequent tRNA. By this modification, the sequences of the inventive mRNA sequence can be modified such that codons for which more frequently occurring tRNAs are available are inserted. In other words, according to the invention, by this modification all codons of the wild type sequence which code for a tRNA which is relatively rare in the cell can in each case be exchanged for a codon which codes for a respective tRNA which is relatively frequent in the cell and which, in each case, carries the same amino acid as the relatively rare tRNA. Furthermore, it is particularly preferable to link the sequential G/C content which is increased, in particular maximized, in the inventive mRNA sequence with the “frequent” codons without modifying the amino acid sequence of the protein encoded by the coding region of the inventive mRNA sequence or of the coding region. This preferred embodiment allows provision of a particularly efficiently translated and stabilized (modified) inventive mRNA sequence.

Substitutions, additions or eliminations of bases are preferably carried out using a DNA matrix for preparation of the nucleic acid molecule by techniques of the well known site directed mutagenesis or with an oligonucleotide ligation. In such a process, for preparation of the inventive mRNA sequence as defined herein a corresponding DNA molecule may be transcribed in vitro. This DNA matrix preferably comprises a suitable promoter, e.g. a T7 or SP6 promoter, for in vitro transcription, which is followed by the desired nucleotide sequence for the inventive mRNA sequence to be prepared and a termination signal for in vitro transcription. The DNA molecule, which forms the matrix of the mRNA of interest, may be prepared by fermentative proliferation and subsequent isolation as part of a plasmid which can be replicated in bacteria. Plasmids which may be mentioned as suitable for the present invention are e.g. the plasmids pT7 Ts (GenBank accession number AB255037.1; Lai et al., Development 1995, 121: 2349 to 2360), pGEM® series, e.g. pGEM®-1 (GenBank accession number X65300; from Promega) and pSP64 (GenBank accession number X65327); cf. also Mezei and Storts, Purification of PCR Products, in: Griffin and Griffin (ed.), PCR Technology: Current Innovation, CRC Press, Boca Raton, F L, 2001.

According to a preferred embodiment, the inventive mRNA comprises a coding region encoding at least one antigenic peptide or protein derived from the glycoprotein (GP) and/or the matrix protein 40 (VP40) and/or the nucleoprotein (NP) of a virus of the genus Ebolavirus or Marburgvirus or a fragment, variant or derivative thereof, preferably as defined herein, wherein the coding region comprises a wild type nucleic acid sequence or a modified wild type nucleic acid sequence, preferably as defined herein. Most preferably, the coding region comprises a nucleic acid sequence corresponding to at least one of SEQ ID NO. 20 to 27 or SEQ ID NO. 53 to 70, or a nucleic acid sequence having at least 80%, more preferably at least 85%, 90%, 95% or 99%, identity with SEQ ID NO. 20 to 27 or SEQ ID NO. 53 to 70.

Alternatively, the inventive mRNA may also comprise a coding region encoding at least one antigenic peptide or protein derived from the glycoprotein (GP) and/or the matrix protein 40 (VP40) and/or the nucleoprotein (NP) of a virus of the genus Ebolavirus or Marburgvirus or a fragment, variant or derivative thereof, preferably as defined herein, wherein the G/C content of the coding region is increased in comparison to the G/C content of the respect wild type mRNA and wherein the amino acid sequence encoded by the coding region is preferably not modified compared to the amino acid sequence encoded by the respective wild type coding region.

According to a preferred embodiment, the inventive mRNA comprises a coding region encoding at least one antigenic peptide or protein derived from the glycoprotein (GP) and/or the matrix protein 40 (VP40) and/or the nucleoprotein (NP) of a virus of the genus Ebolavirus or Marburgvirus or a fragment, variant or derivative thereof, preferably as defined herein, wherein the coding region comprises at least one nucleic acid sequence selected from SEQ ID NO. 71 to 88, SEQ ID NO. 89 to 106, SEQ ID NO. 107 to 124, SEQ ID NO. 125 to 142, SEQ ID NO. 143 to 160, SEQ ID NO. 161 to 178, SEQ ID NO. 179 to 196, SEQ ID NO. 197 to 214, or from SEQ ID NO. 215 to 232.

Alternatively, the inventive mRNA comprises a coding region encoding at least one antigenic peptide or protein derived from the glycoprotein (GP) and/or the matrix protein 40 (VP40) and/or the nucleoprotein (NP) of a virus of the genus Ebolavirus or Marburgvirus or a fragment, variant or derivative thereof, preferably as defined herein, wherein the coding region comprises at least one nucleic acid sequence having at least 80%, more preferably at least 85%, 90%, 95% or 99%, identity with a nucleic acid sequence selected from SEQ ID NO. 71 to 88, SEQ ID NO. 89 to 106, SEQ ID NO. 107 to 124, SEQ ID NO. 125 to 142, SEQ ID NO. 143 to 160, SEQ ID NO. 161 to 178, SEQ ID NO. 179 to 196, SEQ ID NO. 197 to 214, or from SEQ ID NO. 215 to 232.

In a preferred embodiment, the inventive mRNA comprises a coding region encoding at least one antigenic peptide or protein derived from the glycoprotein (GP) of a virus of the genus Ebolavirus or Marburgvirus, or a fragment, variant or derivative thereof, wherein the virus is preferably selected from the species Ebola ebolavirus (EBOV), Bundibugyo ebolavirus (BDBV), Sudan ebolavirus (SUDV), Taï Forest ebolavirus (TAFV) and Marburg marburgvirus (MARV), and wherein the glycoprotein preferably comprises an amino acid sequence according to SEQ ID NO. 1, 2, 3, 4, 5 or 6.

In another embodiment, the inventive mRNA comprises a coding region encoding at least one antigenic peptide or protein derived from the glycoprotein (GP) of a virus of the genus Ebolavirus or Marburgvirus, or a fragment, variant or derivative thereof, wherein the glycoprotein preferably comprises an amino acid sequence according to SEQ ID NO. 1, 2, 3, 4, 5 or 6, and wherein the G/C content of the coding region is increased in comparison to the G/C content of the respect wild type mRNA and wherein the amino acid sequence encoded by the coding region is preferably not modified compared to the amino acid sequence encoded by the respective wild type coding region.

In a further embodiment, the inventive mRNA comprises a coding region encoding at least one antigenic peptide or protein derived from the glycoprotein (GP) of a virus of the genus Ebolavirus or Marburgvirus, or a fragment, variant or derivative thereof, wherein the coding region preferably comprises a nucleic acid sequence corresponding to any one of SEQ ID NO. 53 to 55, 61 to 63, 71 to 73, 79 to 81, 89 to 91, 97 to 99, 107 to 109, 115 to 117, 125 to 127, 133 to 135, 143 to 145, 151 to 153, 161 to 163, 169 to 171, 179 to 181, 187 to 189, 197 to 199, 205 to 207, 215 to 217, or 223 to 225. More preferably, the inventive mRNA comprises a coding region encoding at least one antigenic peptide or protein derived from the glycoprotein (GP) of a virus of the genus Ebolavirus or Marburgvirus, or a fragment, variant or derivative thereof, wherein the coding region preferably comprises at least one nucleic acid sequence having at least 80%, more preferably at least 85%, 90%, 95% or 99%, identity with a nucleic acid sequence selected from any one of SEQ ID NO. 53 to 55, 61 to 63, 71 to 73, 79 to 81, 89 to 91, 97 to 99, 107 to 109, 115 to 117, 125 to 127, 133 to 135, 143 to 145, 151 to 153, 161 to 163, 169 to 171, 179 to 181, 187 to 189, 197 to 199, 205 to 207, 215 to 217, or 223 to 225.

According to a preferred embodiment, the inventive mRNA comprises a coding region encoding at least one antigenic peptide or protein derived from the matrix protein 40 (VP40) of a virus of the genus Ebolavirus or Marburgvirus, or a fragment, variant or derivative thereof, wherein the virus is preferably selected from the species Ebola ebolavirus (EBOV), Bundibugyo ebolavirus (BDBV), Sudan ebolavirus (SUDV), Taï Forest ebolavirus (TAFV) and Marburg marburgvirus (MARV), and wherein the glycoprotein preferably comprises an amino acid sequence according to SEQ ID NO. 7, 8, 9, 10, 11 or 12.

In another embodiment, the inventive mRNA comprises a coding region encoding at least one antigenic peptide or protein derived from the matrix protein 40 (VP40) of a virus of the genus Ebolavirus or Marburgvirus, or a fragment, variant or derivative thereof, wherein the matrix protein 40 (VP40) preferably comprises an amino acid sequence according to SEQ ID NO. 7, 8, 9, 10, 11 or 12, and wherein the G/C content of the coding region is increased in comparison to the G/C content of the respect wild type mRNA and wherein the amino acid sequence encoded by the coding region is preferably not modified compared to the amino acid sequence encoded by the respective wild type coding region.

According to a further embodiment, the inventive mRNA comprises a coding region encoding at least one antigenic peptide or protein derived from the matrix protein 40 (VP40) of a virus of the genus Ebolavirus or Marburgvirus, or a fragment, variant or derivative thereof, wherein the coding region preferably comprises a nucleic acid sequence corresponding to any one of SEQ ID NO. 56 to 58, 64 to 66, 74 to 76, 82 to 84, 92 to 94, 100 to 102, 110 to 112, 118 to 120, 128 to 130, 136 to 138, 146 to 148, 154 to 156, 164 to 166, 172 to 174, 182 to 184, 190 to 192, 200 to 202, 208 to 210, 218 to 220 or 226 to 228. More preferably, the inventive mRNA comprises a coding region encoding at least one antigenic peptide or protein derived from the matrix protein 40 (VP40) of a virus of the genus Ebolavirus or Marburgvirus, or a fragment, variant or derivative thereof, wherein the coding region preferably comprises at least one nucleic acid sequence having at least 80%, more preferably at least 85%, 90%, 95% or 99%, identity with a nucleic acid sequence selected from any one of SEQ ID NO. 56 to 58, 64 to 66, 74 to 76, 82 to 84, 92 to 94, 100 to 102, 110 to 112, 118 to 120, 128 to 130, 136 to 138, 146 to 148, 154 to 156, 164 to 166, 172 to 174, 182 to 184, 190 to 192, 200 to 202, 208 to 210, 218 to 220 or 226 to 228.

In a preferred embodiment, the inventive mRNA comprises a coding region encoding at least one antigenic peptide or protein derived from the nucleoprotein (NP) of a virus of the genus Ebolavirus or Marburgvirus, or a fragment, variant or derivative thereof, wherein the virus is preferably selected from the species Ebola ebolavirus (EBOV), Bundibugyo ebolavirus (BDBV), Sudan ebolavirus (SUDV), Taï Forest ebolavirus (TAFV) and Marburg marburgvirus (MARV), and wherein the nucleoprotein (NP) preferably comprises an amino acid sequence according to SEQ ID NO. 13, 14, 15, 16, 17 or 18.

According to another embodiment, the inventive mRNA comprises a coding region encoding at least one antigenic peptide or protein derived from the nucleoprotein (NP) of a virus of the genus Ebolavirus or Marburgvirus, or a fragment, variant or derivative thereof, wherein the nucleoprotein (NP) preferably comprises an amino acid sequence according to SEQ ID NO. 13, 14, 15, 16, 17 or 18, and wherein the G/C content of the coding region is increased in comparison to the G/C content of the respect wild type mRNA and wherein the amino acid sequence encoded by the coding region is preferably not modified compared to the amino acid sequence encoded by the respective wild type coding region.

In a further embodiment, the inventive mRNA comprises a coding region encoding at least one antigenic peptide or protein derived from the nucleoprotein (NP) of a virus of the genus Ebolavirus or Marburgvirus, or a fragment, variant or derivative thereof, wherein the coding region preferably comprises a nucleic acid sequence corresponding to any one of SEQ ID NO. 59, 60, 67 to 70, 77, 78, 85 to 88, 95, 96, 103 to 106, 113, 114, 121 to 124, 131, 132, 139 to 142, 149, 150, 157 to 160, 167, 168, 175 to 178, 185, 186, 193 to 196, 203, 204, 211 to 214, 221, 222 or 229 to 232. More preferably, the inventive mRNA comprises a coding region encoding at least one antigenic peptide or protein derived from the nucleoprotein (NP) of a virus of the genus Ebolavirus or Marburgvirus, or a fragment, variant or derivative thereof, wherein the coding region preferably comprises at least one nucleic acid sequence having at least 80%, more preferably at least 85%, 90%, 95% or 99%, identity with a nucleic acid sequence selected from any one of SEQ ID NO. 59, 60, 67 to 70, 77, 78, 85 to 88, 95, 96, 103 to 106, 113, 114, 121 to 124, 131, 132, 139 to 142, 149, 150, 157 to 160, 167, 168, 175 to 178, 185, 186, 193 to 196, 203, 204, 211 to 214, 221, 222 or 229 to 232.

According to certain embodiments, the invention provides an mRNA suitable for use in treatment or prophylaxis of an infection with a virus of the species Ebola ebolavirus (EBOV), in particular for use as a vaccine.

Preferably, the inventive mRNA comprises a coding region encoding at least one antigenic peptide or protein derived from the glycoprotein (GP), the matrix protein 40 (VP40), and/or the nucleoprotein (NP) of a virus of the species Ebola ebolavirus (EBOV), or a fragment, variant or derivative thereof, wherein the glycoprotein (GP), the matrix protein 40 (VP40), and/or the nucleoprotein (NP) preferably comprises an amino acid sequence according to SEQ ID NO. 1, 2, 7, 8, 13 or 14, and wherein the coding region preferably comprises a nucleic acid sequence according to any one of SEQ ID NO. 53 to 60. Alternatively, the coding region comprises a nucleic acid sequence having at least 80%, more preferably at least 85%, 90%, 95% or 99%, identity with a nucleic acid sequence according to any one of SEQ ID NO. 53 to 60.

More preferably, the inventive mRNA comprises a coding region encoding at least one antigenic peptide or protein derived from the glycoprotein (GP), the matrix protein 40 (VP40), and/or the nucleoprotein (NP) of a virus of the species Ebola ebolavirus (EBOV), or a fragment, variant or derivative thereof, wherein the glycoprotein (GP), the matrix protein 40 (VP40), and/or the nucleoprotein (NP) preferably comprises an amino acid sequence according to SEQ ID NO. 1, 2, 7, 8, 13 or 14, and wherein the G/C content of the coding region is increased in comparison to the G/C content of the respect wild type mRNA and wherein the amino acid sequence encoded by the coding region is preferably not modified compared to the amino acid sequence encoded by the respective wild type coding region.

According to a particularly preferred embodiment, the inventive mRNA comprises a coding region encoding at least one antigenic peptide or protein derived from the glycoprotein (GP), the matrix protein 40 (VP40), and/or the nucleoprotein (NP) of a virus of the species Ebola ebolavirus (EBOV), wherein the coding region preferably comprises a nucleic acid sequence corresponding to any one of SEQ ID NO. 71, 72, 74, 75, 77, 78, 89, 90, 92, 93, 95, 96, 107, 108, 110, 111, 113, 114, 125, 126, 128, 129, 131, 132, 143, 144, 146, 147, 149, 150, 161, 162, 164, 165, 167, 168, 179, 180, 182, 183, 185, 186, 197, 198, 200, 201, 203, 204, 215, 216, 218, 219, 221 or 222. More preferably, the inventive mRNA comprises a coding region encoding at least one antigenic peptide or protein derived from the glycoprotein (GP), the matrix protein 40 (VP40), and/or the nucleoprotein (NP) of a virus of the species Ebola ebolavirus (EBOV), wherein the coding region preferably comprises at least one nucleic acid sequence having at least 80%, more preferably at least 85%, 90%, 95% or 99%, identity with a nucleic acid sequence selected from any one of SEQ ID NO. 71, 72, 74, 75, 77, 78, 89, 90, 92, 93, 95, 96, 107, 108, 110, 111, 113, 114, 125, 126, 128, 129, 131, 132, 143, 144, 146, 147, 149, 150, 161, 162, 164, 165, 167, 168, 179, 180, 182, 183, 185, 186, 197, 198, 200, 201, 203, 204, 215, 216, 218, 219, 221 or 222.

According to a preferred embodiment, the inventive mRNA comprises a coding region encoding at least one antigenic peptide or protein derived from the glycoprotein (GP) of a virus of the species Ebola ebolavirus (EBOV), or a fragment, variant or derivative thereof, wherein the glycoprotein (GP) preferably comprises an amino acid sequence according to SEQ ID NO. 1 or 2, and wherein the coding region preferably comprises a nucleic acid sequence according to any one of SEQ ID NO. 53 or 54. Alternatively, the coding region comprises a nucleic acid sequence having at least 80%, more preferably at least 85%, 90%, 95% or 99%, identity with a nucleic acid sequence according to any one of SEQ ID NO. 53 or 54.

More preferably, the inventive mRNA comprises a coding region encoding at least one antigenic peptide or protein derived from the glycoprotein (GP) of a virus of the species Ebola ebolavirus (EBOV), or a fragment, variant or derivative thereof, wherein the glycoprotein (GP) preferably comprises an amino acid sequence according to SEQ ID NO. 1 or 2 and wherein the G/C content of the coding region is increased in comparison to the G/C content of the respect wild type mRNA and wherein the amino acid sequence encoded by the coding region is preferably not modified compared to the amino acid sequence encoded by the respective wild type coding region.

According to a particularly preferred embodiment, the inventive mRNA comprises a coding region encoding at least one antigenic peptide or protein derived from the glycoprotein (GP) of a virus of the species Ebola ebolavirus (EBOV), wherein the coding region preferably comprises a nucleic acid sequence corresponding to any one of SEQ ID NO. 71, 72, 89, 90, 107, 108, 125, 126, 143, 144, 161, 162, 179, 180, 197, 198, 215 or 216. More preferably, the inventive mRNA comprises a coding region encoding at least one antigenic peptide or protein derived from the glycoprotein (GP) of a virus of the species Ebola ebolavirus (EBOV), wherein the coding region preferably comprises at least one nucleic acid sequence having at least 80%, more preferably at least 85%, 90%, 95% or 99%, identity with a nucleic acid sequence selected from any one of SEQ ID NO. 71, 72, 89, 90, 107, 108, 125, 126, 143, 144, 161, 162, 179, 180, 197, 198, 215 or 216.

Preferably, the inventive mRNA comprises the nucleic acid sequence according to SEQ ID NO. 45 or 46.

In other embodiments, the invention provides an mRNA suitable for use in treatment or prophylaxis of an infection with a virus of the species Bundibugyo ebolavirus (BDBV), in particular for use as a vaccine.

Preferably, the inventive mRNA comprises a coding region encoding at least one antigenic peptide or protein derived from the glycoprotein (GP), the matrix protein 40 (VP40), and/or the nucleoprotein (NP) of a virus of the species Bundibugyo ebolavirus (BDBV), or a fragment, variant or derivative thereof, wherein the glycoprotein (GP), the matrix protein 40 (VP40), and/or the nucleoprotein (NP) preferably comprises an amino acid sequence according to SEQ ID NO. 4, 10 or 16, and wherein the coding region preferably comprises a nucleic acid sequence according to any one of SEQ ID NO. 61, 64 or 68. Alternatively, the coding region comprises a nucleic acid sequence having at least 80%, more preferably at least 85%, 90%, 95% or 99%, identity with a nucleic acid sequence according to any one of SEQ ID NO. 61, 64 or 68.

More preferably, the inventive mRNA comprises a coding region encoding at least one antigenic peptide or protein derived from the glycoprotein (GP), the matrix protein 40 (VP40), and/or the nucleoprotein (NP) of a virus of the species Bundibugyo ebolavirus (BDBV), or a fragment, variant or derivative thereof, wherein the glycoprotein (GP), the matrix protein 40 (VP40), and/or the nucleoprotein (NP) preferably comprises an amino acid sequence according to SEQ ID NO. 4, 10 or 16, and wherein the G/C content of the coding region is increased in comparison to the G/C content of the respect wild type mRNA and wherein the amino acid sequence encoded by the coding region is preferably not modified compared to the amino acid sequence encoded by the respective wild type coding region.

According to a particularly preferred embodiment, the inventive mRNA comprises a coding region encoding at least one antigenic peptide or protein derived from the glycoprotein (GP), the matrix protein 40 (VP40), and/or the nucleoprotein (NP) of a virus of the species Bundibugyo ebolavirus (BDBV), wherein the coding region preferably comprises a nucleic acid sequence corresponding to any one of SEQ ID NO. 79, 82, 86, 97, 100, 104, 115, 118, 122, 133, 136, 140, 151, 154, 158, 169, 172, 176, 187, 190, 194, 205, 208, 212, 223, 226 or 230. More preferably, the inventive mRNA comprises a coding region encoding at least one antigenic peptide or protein derived from the glycoprotein (GP), the matrix protein 40 (VP40), and/or the nucleoprotein (NP) of a virus of the species Bundibugyo ebolavirus (BDBV), wherein the coding region preferably comprises at least one nucleic acid sequence having at least 80%, more preferably at least 85%, 90%, 95% or 99%, identity with a nucleic acid sequence selected from any one of SEQ ID NO. 79, 82, 86, 97, 100, 104, 115, 118, 122, 133, 136, 140, 151, 154, 158, 169, 172, 176, 187, 190, 194, 205, 208, 212, 223, 226 or 230.

According to certain embodiments, the invention provides an mRNA suitable for use in treatment or prophylaxis of an infection with a virus of the species Sudan ebolavirus (SUDV), in particular for use as a vaccine.

Preferably, the inventive mRNA comprises a coding region encoding at least one antigenic peptide or protein derived from the glycoprotein (GP), the matrix protein 40 (VP40), and/or the nucleoprotein (NP) of a virus of the species Sudan ebolavirus (SUDV), or a fragment, variant or derivative thereof, wherein the glycoprotein (GP), the matrix protein 40 (VP40), and/or the nucleoprotein (NP) preferably comprises an amino acid sequence according to SEQ ID NO. 5, 11 or 17, and wherein the coding region preferably comprises a nucleic acid sequence according to any one of SEQ ID NO. 62, 65 or 69. Alternatively, the coding region comprises a nucleic acid sequence having at least 80%, more preferably at least 85%, 90%, 95% or 99%, identity with a nucleic acid sequence according to any one of SEQ ID NO. 62, 65 or 69.

More preferably, the inventive mRNA comprises a coding region encoding at least one antigenic peptide or protein derived from the glycoprotein (GP), the matrix protein 40 (VP40), and/or the nucleoprotein (NP) of a virus of the species Sudan ebolavirus (SUDV), or a fragment, variant or derivative thereof, wherein the glycoprotein (GP), the matrix protein 40 (VP40), and/or the nucleoprotein (NP) preferably comprises an amino acid sequence according to SEQ ID NO. 5, 11 or 17, and wherein the G/C content of the coding region is increased in comparison to the G/C content of the respect wild type mRNA and wherein the amino acid sequence encoded by the coding region is preferably not modified compared to the amino acid sequence encoded by the respective wild type coding region.

According to a particularly preferred embodiment, the inventive mRNA comprises a coding region encoding at least one antigenic peptide or protein derived from the glycoprotein (GP), the matrix protein 40 (VP40), and/or the nucleoprotein (NP) of a virus of the species Sudan ebolavirus (SUDV), wherein the coding region preferably comprises a nucleic acid sequence corresponding to any one of SEQ ID NO. 80, 83, 87, 98, 101, 105, 116, 119, 123, 134, 137, 141, 152, 155, 159, 170, 173, 177, 188, 191, 195, 206, 209, 213, 224, 227 or 231. More preferably, the inventive mRNA comprises a coding region encoding at least one antigenic peptide or protein derived from the glycoprotein (GP), the matrix protein 40 (VP40), and/or the nucleoprotein (NP) of a virus of the species Sudan ebolavirus (SUDV), wherein the coding region preferably comprises at least one nucleic acid sequence having at least 80%, more preferably at least 85%, 90%, 95% or 99%, identity with a nucleic acid sequence selected from any one of SEQ ID NO. 80, 83, 87, 98, 101, 105, 116, 119, 123, 134, 137, 141, 152, 155, 159, 170, 173, 177, 188, 191, 195, 206, 209, 213, 224, 227 or 231.

According to further embodiments, the invention provides an mRNA suitable for use in treatment or prophylaxis of an infection with a virus of the species Taï Forest ebolavirus (TAFV), in particular for use as a vaccine.

Preferably, the inventive mRNA comprises a coding region encoding at least one antigenic peptide or protein derived from the glycoprotein (GP), the matrix protein 40 (VP40), and/or the nucleoprotein (NP) of a virus of the species Taï Forest ebolavirus (TAFV), or a fragment, variant or derivative thereof, wherein the glycoprotein (GP), the matrix protein 40 (VP40), and/or the nucleoprotein (NP) preferably comprises an amino acid sequence according to SEQ ID NO. 6, 12 or 18, and wherein the coding region preferably comprises a nucleic acid sequence according to any one of SEQ ID NO. 63, 66 or 70. Alternatively, the coding region comprises a nucleic acid sequence having at least 80%, more preferably at least 85%, 90%, 95% or 99%, identity with a nucleic acid sequence according to any one of SEQ ID NO. 63, 66 or 70.

More preferably, the inventive mRNA comprises a coding region encoding at least one antigenic peptide or protein derived from the glycoprotein (GP), the matrix protein 40 (VP40), and/or the nucleoprotein (NP) of a virus of the species Taï Forest ebolavirus (TAFV), or a fragment, variant or derivative thereof, wherein the glycoprotein (GP), the matrix protein 40 (VP40), and/or the nucleoprotein (NP) preferably comprises an amino acid sequence according to SEQ ID NO. 6, 12 or 18, and wherein the G/C content of the coding region is increased in comparison to the G/C content of the respect wild type mRNA and wherein the amino acid sequence encoded by the coding region is preferably not modified compared to the amino acid sequence encoded by the respective wild type coding region.

According to a particularly preferred embodiment, the inventive mRNA comprises a coding region encoding at least one antigenic peptide or protein derived from the glycoprotein (GP), the matrix protein 40 (VP40), and/or the nucleoprotein (NP) of a virus of the species Taï Forest ebolavirus (TAFV), wherein the coding region preferably comprises a nucleic acid sequence corresponding to any one of SEQ ID NO. 81, 84, 88, 99, 102, 106, 117, 120, 124, 135, 138, 142, 153, 156, 160, 171, 174, 178, 189, 192, 196, 207, 210, 214, 225, 228 or 232. More preferably, the inventive mRNA comprises a coding region encoding at least one antigenic peptide or protein derived from the glycoprotein (GP), the matrix protein 40 (VP40), and/or the nucleoprotein (NP) of a virus of the species Taï Forest ebolavirus (TAFV), wherein the coding region preferably comprises at least one nucleic acid sequence having at least 80%, more preferably at least 85%, 90%, 95% or 99%, identity with a nucleic acid sequence selected from any one of SEQ ID NO. 81, 84, 88, 99, 102, 106, 117, 120, 124, 135, 138, 142, 153, 156, 160, 171, 174, 178, 189, 192, 196, 207, 210, 214, 225, 228 or 232.

In other embodiments, the invention provides an mRNA suitable for use in treatment or prophylaxis of an infection with a virus of the species Marburg marburgvirus (MARV), in particular for use as a vaccine.

Preferably, the inventive mRNA comprises a coding region encoding at least one antigenic peptide or protein derived from the glycoprotein (GP), the matrix protein 40 (VP40), and/or the nucleoprotein (NP) of a virus of the species Marburg marburgvirus (MARV), or a fragment, variant or derivative thereof, wherein the glycoprotein (GP), the matrix protein 40 (VP40), and/or the nucleoprotein (NP) preferably comprises an amino acid sequence according to SEQ ID NO. 3, 9 or 15, and wherein the coding region preferably comprises a nucleic acid sequence according to any one of SEQ ID NO. 55, 58 or 67. Alternatively, the coding region comprises a nucleic acid sequence having at least 80%, more preferably at least 85%, 90%, 95% or 99%, identity with a nucleic acid sequence according to any one of SEQ ID NO. 55, 58 or 67.

More preferably, the inventive mRNA comprises a coding region encoding at least one antigenic peptide or protein derived from the glycoprotein (GP), the matrix protein 40 (VP40), and/or the nucleoprotein (NP) of a virus of the species Marburg marburgvirus (MARV), or a fragment, variant or derivative thereof, wherein the glycoprotein (GP), the matrix protein 40 (VP40), and/or the nucleoprotein (NP) preferably comprises an amino acid sequence according to SEQ ID NO. 3, 9 or 15, and wherein the G/C content of the coding region is increased in comparison to the G/C content of the respect wild type mRNA and wherein the amino acid sequence encoded by the coding region is preferably not modified compared to the amino acid sequence encoded by the respective wild type coding region.

According to a particularly preferred embodiment, the inventive mRNA comprises a coding region encoding at least one antigenic peptide or protein derived from the glycoprotein (GP), the matrix protein 40 (VP40), and/or the nucleoprotein (NP) of a virus of the species Marburg marburgvirus (MARV), wherein the coding region preferably comprises a nucleic acid sequence corresponding to any one of SEQ ID NO. 73, 76, 85, 91, 94, 103, 109, 112, 121, 127, 130, 139, 145, 148, 157, 163, 166, 175, 181, 184, 193, 199, 202, 211, 217, 220 or 229. More preferably, the inventive mRNA comprises a coding region encoding at least one antigenic peptide or protein derived from the glycoprotein (GP), the matrix protein 40 (VP40), and/or the nucleoprotein (NP) of a virus of the species Marburg marburgvirus (MARV), wherein the coding region preferably comprises at least one nucleic acid sequence having at least 80%, more preferably at least 85%, 90%, 95% or 99%, identity with a nucleic acid sequence selected from any one of SEQ ID NO. 73, 76, 85, 91, 94, 103, 109, 112, 121, 127, 130, 139, 145, 148, 157, 163, 166, 175, 181, 184, 193, 199, 202, 211, 217, 220 or 229.

In a particularly preferred embodiment, the inventive mRNA sequence according to the first aspect of the present invention comprises, preferably in 5′- to 3′-direction:
a) a 5′-CAP structure, as defined herein, preferably m7GpppN;
b) a coding region with an increased or even maximized G/C content compared with the G/C content of the coding region of the wild type mRNA, encoding at least one antigenic peptide or protein derived the glycoprotein (GP) and/or the matrix protein 40 (VP40) and/or the nucleoprotein (NP) of a virus of the genus Ebolavirus or Marburgvirus or a fragment, variant or derivative thereof;
c) a 3′-UTR element as defined herein, preferably derived of a gene providing a stable mRNA, most preferably the corresponding RNA sequence of the nucleic acid sequence according to SEQ ID NO. 33, or a homolog, a fragment or variant thereof;
d) a poly(A) sequence, preferably consisting of 64 adenosines
e) optionally a poly(C) sequence, preferably consisting of 30 cytosines.
f) at least one histone stem-loop sequence, preferably the corresponding RNA sequence of the nucleic acid sequence according to SEQ ID NO. 35.

In a further particularly preferred embodiment, the inventive mRNA sequence according to the first aspect of the present invention comprises preferably in 5′ to 3′ direction:
a) a 5′-CAP structure, as defined herein, preferably m7GpppN;
b) a 5′-UTR element as defined herein, preferably a 5′-UTR element which comprises or consists of a nucleic acid sequence which is derived from the 5′-UTR of a TOP gene, preferably the 5′-UTR of human ribosomal protein Large 32 lacking the 5′ terminal oligopyrimidine tract according to SEQ ID NO. 32 or the corresponding RNA sequence; or a fragment, homolog or variant thereof;
c) a coding region, preferably with an increased or even maximized G/C content compared with the G/C content of the coding region of the wild type mRNA, encoding at least one antigenic peptide or protein derived from the glycoprotein (GP) and/or the matrix protein 40 (VP40) and/or the nucleoprotein (NP) of a virus of the genus Ebolavirus or Marburgvirus or a fragment, variant or derivative thereof;
d) a 3′-UTR element, preferably the 3′-UTR element of human albumin according to SEQ ID NO. 33 or the corresponding RNA, or a homolog, a fragment or a variant thereof;
e) a poly(A) sequence, preferably consisting of 64 adenosines
f) optionally a poly(C) sequence, preferably consisting of 30 cytosines.
g) at least one histone stem-loop sequence, preferably the corresponding RNA sequence of the nucleic acid sequence according to SEQ ID NO. 35.

Most preferably, the inventive mRNA sequence comprises or consists of corresponding mRNA sequences of the following optimised nucleotide sequences (GC optimised nucleotide sequence with UTRs 5′-UTR: 32L TOP UTR, 3′-UTR: albumin7-A64-N5-C30-histoneSL-N5).

The following optimised nucleotide sequence (corresponding to the optimized mRNA sequence according to the invention) according to SEQ ID NO. 37 corresponds to the amino acid sequence according to SEQ ID NO. 1 and refers to the glycoprotein of an Ebolavirus strain EBOV isolated in an outbreak from 1976 in Mayinga, Zaire as described above.

EBOV GP, Mayinga, Zaire 1976
Optimised nucleotide sequence (SEQ ID NO. 37):
GGGGCGCTGCCTACGGAGGTGGCAGCCATCTCCTTCTCGGCATCAAGCTT
ACCATGGGCGTGACCGGGATCCTGCAGCTCCCCCGCGACCGGTTCAAGCG
CACCAGCTTCTTCCTGTGGGTCATCATCCTGTTCCAGCGGACGTTCTCCA
TCCCGCTCGGCGTGATCCACAACAGCACCCTGCAGGTGTCCGACGTCGAC
AAGCTGGTGTGCCGCGACAAGCTCAGCTCCACCAACCAGCTGCGGAGCGT
GGGGCTGAACCTCGAGGGCAACGGGGTCGCCACCGACGTGCCCTCCGCCA
CGAAGCGCTGGGGCTTCCGGAGCGGCGTGCCGCCCAAGGTCGTGAACTAC
GAGGCGGGGGAGTGGGCCGAGAACTGCTACAACCTGGAGATCAAGAAGCC
CGACGGCTCCGAGTGCCTGCCCGCCGCCCCCGACGGGATCCGCGGCTTCC
CCCGGTGCCGCTACGTGCACAAGGTCAGCGGGACCGGCCCGTGCGCCGGC
GACTTCGCGTTCCACAAGGAGGGGGCCTTCTTCCTCTACGACCGGCTGGC
CTCCACCGTGATCTACCGCGGCACCACGTTCGCCGAGGGGGTGGTCGCGT
TCCTGATCCTCCCCCAGGCCAAGAAGGACTTCTTCAGCTCCCACCCCCTG
CGGGAGCCCGTGAACGCCACCGAGGACCCGAGCTCCGGCTACTACAGCAC
CACCATCCGCTACCAGGCCACGGGCTTCGGGACCAACGAGACCGAGTACC
TGTTCGAGGTGGACAACCTCACCTACGTCCAGCTGGAGTCCCGGTTCACG
CCCCAGTTCCTGCTCCAGCTGAACGAGACCATCTACACCAGCGGCAAGCG
CTCCAACACCACGGGGAAGCTGATCTGGAAGGTGAACCCCGAGATCGACA
CCACCATCGGCGAGTGGGCCTTCTGGGAGACCAAGAAGAACCTCACGCGG
AAGATCCGCAGCGAGGAGCTGAGCTTCACCGTGGTCTCCAACGGGGCGAA
GAACATCAGCGGCCAGTCCCCCGCCCGGACCAGCTCCGACCCGGGCACCA
ACACGACCACCGAGGACCACAAGATCATGGCCAGCGAGAACTCCAGCGCC
ATGGTGCAGGTGCACTCCCAGGGGCGCGAGGCCGCGGTCAGCCACCTGAC
CACGCTCGCCACCATCTCCACCAGCCCCCAGTCCCTGACCACGAAGCCCG
GCCCCGACAACAGCACCCACAACACCCCGGTGTACAAGCTGGACATCTCC
GAGGCCACCCAGGTCGAGCAGCACCACCGGCGCACCGACAACGACAGCAC
GGCCTCCGACACCCCCAGCGCCACCACCGCGGCCGGGCCGCCCAAGGCCG
AGAACACGAACACCTCCAAGAGCACCGACTTCCTCGACCCCGCCACCACG
ACCAGCCCCCAGAACCACTCCGAGACCGCCGGCAACAACAACACCCACCA
CCAGGACACGGGGGAGGAGAGCGCGTCCAGCGGCAAGCTGGGCCTGATCA
CCAACACCATCGCCGGGGTGGCCGGCCTCATCACCGGGGGCCGCCGGACG
CGCCGGGAGGCCATCGTGAACGCGCAGCCCAAGTGCAACCCCAACCTGCA
CTACTGGACCACCCAGGACGAGGGGGCCGCCATCGGCCTGGCCTGGATCC
CGTACTTCGGCCCCGCCGCGGAGGGGATCTACATCGAGGGCCTCATGCAC
AACCAGGACGGGCTGATCTGCGGCCTGCGCCAGCTCGCCAACGAGACCAC
GCAGGCCCTGCAGCTGTTCCTCCGGGCCACCACCGAGCTGCGCACCTTCT
CCATCCTGAACCGGAAGGCCATCGACTTCCTCCTGCAGCGCTGGGGCGGG
ACGTGCCACATCCTGGGCCCCGACTGCTGCATCGAGCCGCACGACTGGAC
CAAGAACATCACCGACAAGATCGACCAGATCATCCACGACTTCGTCGACA
AGACCCTGCCCGACCAGGGGGACAACGACAACTGGTGGACGGGCTGGCGG
CAGTGGATCCCCGCGGGGATCGGCGTGACCGGCGTGATCATCGCCGTCAT
CGCCCTCTTCTGCATCTGCAAGTTCGTGTTCTGAGGACTAGTGCATCACA
TTTAAAAGCATCTCAGCCTACCATGAGAATAAGAGAAAGAAAATGAAGAT
CAATAGCTTATTCATCTCTTTTTCTTTTTCGTTGGTGTAAAGCCAACACC
CTGTCTAAAAAACATAAATTTCTTTAATCATTTTGCCTCTTTTCTCTGTG
CTTCAATTAATAAAAAATGGAAAGAACCTAGATCTAAAAAAAAAAAAAAA
AAAAAAAAAAAAAAAAAAAAAAAAAAAAAAAAAAAAAAAAAAAAAAAAAT
GCATCCCCCCCCCCCCCCCCCCCCCCCCCCCCCCCAAAGGCTCTTTTCAG
AGCCACCAGAATT

The RNA sequence corresponding to SEQ ID NO. 37 is defined by SEQ ID NO. 45.

The following optimised nucleotide sequence (corresponding to the optimized mRNA sequence according to the invention) according to SEQ ID NO. 38 corresponds to the amino acid sequence according to SEQ ID NO. 2 and refers to the glycoprotein of an Ebolavirus strain EBOV isolated in an outbreak from 2014 in Sierra Leone as described above.

EBOV GP, Sierra Leone 2014
Optimised nucleotide sequence (SEQ ID NO. 38):
GGGGCGCTGCCTACGGAGGTGGCAGCCATCTCCTTCTCGGCATCAAGCTT
ACCATGGGCGTGACCGGGATCCTGCAGCTCCCCCGCGACCGGTTCAAGCG
CACCAGCTTCTTCCTGTGGGTCATCATCCTGTTCCAGCGGACGTTCTCCA
TCCCGCTCGGCGTGATCCACAACAGCACCCTGCAGGTGTCCGACGTCGAC
AAGCTGGTGTGCCGCGACAAGCTCAGCTCCACCAACCAGCTGCGGAGCGT
GGGGCTGAACCTCGAGGGCAACGGGGTCGCCACCGACGTGCCCTCCGTGA
CGAAGCGCTGGGGCTTCCGGAGCGGCGTCCCGCCCAAGGTGGTGAACTAC
GAGGCCGGGGAGTGGGCGGAGAACTGCTACAACCTGGAGATCAAGAAGCC
CGACGGCTCCGAGTGCCTGCCCGCCGCCCCCGACGGGATCCGCGGCTTCC
CCCGGTGCCGCTACGTCCACAAGGTGAGCGGGACCGGCCCGTGCGCCGGC
GACTTCGCCTTCCACAAGGAGGGGGCGTTCTTCCTCTACGACCGGCTGGC
CTCCACCGTGATCTACCGCGGCACCACGTTCGCCGAGGGGGTCGTGGCCT
TCCTGATCCTCCCCCAGGCGAAGAAGGACTTCTTCAGCTCCCACCCCCTG
CGGGAGCCCGTGAACGCCACCGAGGACCCGAGCTCCGGCTACTACAGCAC
CACCATCCGCTACCAGGCCACGGGCTTCGGGACCAACGAGACCGAGTACC
TGTTCGAGGTCGACAACCTCACCTACGTGCAGCTGGAGTCCCGGTTCACG
CCCCAGTTCCTGCTCCAGCTGAACGAGACCATCTACGCCAGCGGCAAGCG
CTCCAACACCACCGGGAAGCTGATCTGGAAGGTGAACCCCGAGATCGACA
CGACCATCGGCGAGTGGGCCTTCTGGGAGACCAAGAAGAACCTCACCCGG
AAGATCCGCAGCGAGGAGCTGAGCTTCACGGCGGTCTCCAACGGGCCCAA
GAACATCAGCGGCCAGTCCCCGGCCCGGACCAGCTCCGACCCCGAGACCA
ACACCACGAACGAGGACCACAAGATCATGGCCAGCGAGAACTCCAGCGCC
ATGGTGCAGGTGCACTCCCAGGGCCGCAAGGCCGCGGTCAGCCACCTGAC
CACCCTCGCCACCATCTCCACGAGCCCCCAGCCCCCGACCACCAAGACCG
GGCCCGACAACTCCACGCACAACACCCCCGTGTACAAGCTGGACATCAGC
GAGGCCACCCAGGTCGGCCAGCACCACCGGCGCGCCGACAACGACTCCAC
CGCCAGCGACACCCCGCCGGCGACGACCGCCGCCGGGCCCCTGAAGGCCG
AGAACACCAACACCTCCAAGAGCGCCGACTCCCTCGACCTGGCGACGACC
ACCAGCCCCCAGAACTACAGCGAGACCGCCGGCAACAACAACACGCACCA
CCAGGACACCGGGGAGGAGTCCGCCAGCTCCGGCAAGCTGGGCCTCATCA
CCAACACCATCGCCGGGGTGGCGGGCCTGATCACGGGCGGGCGCCGGACC
CGCCGGGAGGTGATCGTCAACGCCCAGCCCAAGTGCAACCCGAACCTGCA
CTACTGGACCACCCAGGACGAGGGGGCCGCCATCGGCCTCGCCTGGATCC
CCTACTTCGGCCCCGCGGCCGAGGGGATCTACACGGAGGGCCTGATGCAC
AACCAGGACGGGCTGATCTGCGGCCTCCGCCAGCTGGCCAACGAGACCAC
CCAGGCCCTGCAGCTCTTCCTGCGGGCCACCACGGAGCTGCGCACCTTCA
GCATCCTCAACCGGAAGGCGATCGACTTCCTGCTGCAGCGCTGGGGCGGG
ACCTGCCACATCCTGGGCCCGGACTGCTGCATCGAGCCCCACGACTGGAC
CAAGAACATCACGGACAAGATCGACCAGATCATCCACGACTTCGTGGACA
AGACCCTCCCCGACCAGGGGGACAACGACAACTGGTGGACCGGCTGGCGG
CAGTGGATCCCCGCCGGGATCGGCGTGACCGGCGTCATCATCGCCGTGAT
CGCCCTGTTCTGCATCTGCAAGTTCGTGTTCTGAGGACTAGTGCATCACA
TTTAAAAGCATCTCAGCCTACCATGAGAATAAGAGAAAGAAAATGAAGAT
CAATAGCTTATTCATCTCTTTTTCTTTTTCGTTGGTGTAAAGCCAACACC
CTGTCTAAAAAACATAAATTTCTTTAATCATTTTGCCTCTTTTCTCTGTG
CTTCAATTAATAAAAAATGGAAAGAACCTAGATCTAAAAAAAAAAAAAAA
AAAAAAAAAAAAAAAAAAAAAAAAAAAAAAAAAAAAAAAAAAAAAAAAAT
GCATCCCCCCCCCCCCCCCCCCCCCCCCCCCCCCCAAAGGCTCTTTTCAG
AGCCACCAGAATT

The RNA sequence corresponding to SEQ ID NO. 38 is defined by SEQ ID NO. 46.

The following optimised nucleotide sequence (corresponding to the optimized mRNA sequence according to the invention) according to SEQ ID NO. 39 corresponds to the amino acid sequence according to SEQ ID NO. 3 and refers to the glycoprotein of a Marburgvirus strain MARV isolated in Angola in 2005 as described above.

MARV GP, Angola 2005
Optimised nucleotide sequence (SEQ ID NO. 39):
GGGGCGCTGCCTACGGAGGTGGCAGCCATCTCCTTCTCGGCATCAAGCTT
ACCATGAAGACCACCTGCCTGCTCATCAGCCTGATCCTGATCCAGGGCGT
GAAGACGCTCCCCATCCTGGAGATCGCCTCCAACATCCAGCCCCAGAACG
TCGACAGCGTGTGCTCCGGGACCCTGCAGAAGACCGAGGACGTGCACCTC
ATGGGCTTCACCCTGAGCGGGCAGAAGGTCGCCGACTCCCCGCTGGAGGC
GAGCAAGCGCTGGGCCTTCCGGGCCGGCGTGCCGCCCAAGAACGTGGAGT
ACACGGAGGGGGAGGAGGCCAAGACCTGCTACAACATCTCCGTCACCGAC
CCCAGCGGCAAGTCCCTCCTGCTGGACCCGCCCACCAACATCCGCGACTA
CCCCAAGTGCAAGACGATCCACCACATCCAGGGCCAGAACCCGCACGCCC
AGGGGATCGCGCTCCACCTGTGGGGCGCCTTCTTCCTGTACGACCGGATC
GCCAGCACCACCATGTACCGCGGGAAGGTGTTCACCGAGGGCAACATCGC
CGCGATGATCGTGAACAAGACGGTCCACAAGATGATCTTCTCCCGGCAGG
GGCAGGGCTACCGCCACATGAACCTCACCAGCACCAACAAGTACTGGACC
TCCAGCAACGGCACGCAGACCAACGACACCGGGTGCTTCGGCACCCTGCA
GGAGTACAACTCCACGAAGAACCAGACCTGCGCCCCCAGCAAGAAGCCCC
TGCCCCTCCCGACCGCCCACCCCGAGGTGAAGCTGACCTCCACGAGCACC
GACGCCACCAAGCTGAACACCACGGACCCCAACTCCGACGACGAGGACCT
CACCACCAGCGGGAGCGGCTCCGGCGAGCAGGAGCCCTACACCACGAGCG
ACGCCGCGACCAAGCAGGGGCTGTCCAGCACCATGCCGCCCACCCCGTCC
CCGCAGCCCAGCACGCCCCAGCAGGGCGGGAACAACACCAACCACTCCCA
GGGCGTGGTCACCGAGCCCGGGAAGACCAACACCACGGCCCAGCCCAGCA
TGCCGCCCCACAACACCACCACCATCTCCACGAACAACACCAGCAAGCAC
AACCTGTCCACCCCCAGCGTGCCCATCCAGAACGCCACCAACTACAACAC
GCAGTCCACCGCCCCGGAGAACGAGCAGACCAGCGCCCCCTCCAAGACCA
CGCTCCTGCCCACCGAGAACCCGACCACCGCGAAGAGCACGAACTCCACC
AAGAGCCCCACCACCACGGTGCCCAACACCACCAACAAGTACTCCACCAG
CCCCAGCCCGACGCCCAACTCCACCGCCCAGCACCTGGTCTACTTCCGGC
GCAAGCGGAACATCCTCTGGCGCGAGGGCGACATGTTCCCCTTCCTGGAC
GGCCTGATCAACGCCCCCATCGACTTCGACCCGGTGCCCAACACCAAGAC
CATCTTCGACGAGAGCTCCAGCTCCGGGGCCAGCGCCGAGGAGGACCAGC
ACGCGTCCCCCAACATCAGCCTCACGCTGTCCTACTTCCCCAAGGTGAAC
GAGAACACCGCCCACAGCGGCGAGAACGAGAACGACTGCGACGCCGAGCT
GCGGATCTGGTCCGTCCAGGAGGACGACCTCGCCGCCGGGCTGAGCTGGA
TCCCGTTCTTCGGCCCCGGGATCGAGGGCCTGTACACCGCGGGCCTCATC
AAGAACCAGAACAACCTGGTGTGCCGCCTGCGGCGCCTCGCCAACCAGAC
CGCCAAGTCCCTGGAGCTGCTCCTGCGGGTGACGACCGAGGAGCGCACCT
TCAGCCTGATCAACCGGCACGCCATCGACTTCCTCCTGGCGCGCTGGGGC
GGGACCTGCAAGGTCCTGGGGCCCGACTGCTGCATCGGCATCGAGGACCT
GTCCCGGAACATCAGCGAGCAGATCGACCAGATCAAGAAGGACGAGCAGA
AGGAGGGGACGGGCTGGGGCCTCGGGGGCAAGTGGTGGACCTCCGACTGG
GGCGTGCTGACCAACCTGGGGATCCTCCTGCTGCTCAGCATCGCCGTGCT
GATCGCCCTGAGCTGCATCTGCCGCATCTTCACCAAGTACATCGGCTGAG
GACTAGTGCATCACATTTAAAAGCATCTCAGCCTACCATGAGAATAAGAG
AAAGAAAATGAAGATCAATAGCTTATTCATCTCTTTTTCTTTTTCGTTGG
TGTAAAGCCAACACCCTGTCTAAAAAACATAAATTTCTTTAATCATTTTG
CCTCTTTTCTCTGTGCTTCAATTAATAAAAAATGGAAAGAACCTAGATCT
AAAAAAAAAAAAAAAAAAAAAAAAAAAAAAAAAAAAAAAAAAAAAAAAAA
AAAAAAAAAAAAAATGCATCCCCCCCCCCCCCCCCCCCCCCCCCCCCCCC
AAAGGCTCTTTTCAGAGCCACCAGAATT

The RNA sequence corresponding to SEQ ID NO. 39 is defined by SEQ ID NO. 47.

The following optimised nucleotide sequence (corresponding to the optimized mRNA sequence according to the invention) according to SEQ ID NO. 40 corresponds to the amino acid sequence according to SEQ ID NO. 7 and refers to the matrix protein VP40 of an Ebolavirus strain EBOV isolated in Zaire in 1976 as described above.

EBOV VP40, Mayinga, Zaire 1976
Optimised nucleotide sequence (SEQ ID NO. 40):
GGGGCGCTGCCTACGGAGGTGGCAGCCATCTCCTTCTCGGCATCAAGCTT
ACCATGCGCCGGGTGATCCTGCCCACCGCCCCGCCCGAGTACATGGAGGC
CATCTACCCCGTCCGCAGCAACTCCACCATCGCGCGGGGCGGGAACAGCA
ACACGGGCTTCCTCACCCCCGAGTCCGTGAACGGGGACACCCCGAGCAAC
CCCCTGCGCCCCATCGCCGACGACACCATCGACCACGCCTCCCACACGCC
CGGCAGCGTGTCCAGCGCCTTCATCCTGGAGGCCATGGTCAACGTGATCT
CCGGGCCGAAGGTGCTCATGAAGCAGATCCCCATCTGGCTGCCCCTGGGC
GTCGCGGACCAGAAGACCTACAGCTTCGACTCCACCACCGCCGCCATCAT
GCTCGCCAGCTACACGATCACCCACTTCGGCAAGGCGACCAACCCCCTGG
TGCGGGTGAACCGCCTGGGGCCGGGCATCCCCGACCACCCCCTCCGGCTG
CTGCGCATCGGGAACCAGGCCTTCCTCCAGGAGTTCGTCCTGCCCCCGGT
GCAGCTGCCCCAGTACTTCACCTTCGACCTCACGGCCCTGAAGCTGATCA
CCCAGCCCCTCCCCGCCGCCACCTGGACCGACGACACGCCGACCGGCTCC
AACGGGGCGCTGCGGCCCGGCATCAGCTTCCACCCCAAGCTGCGCCCCAT
CCTCCTGCCGAACAAGTCCGGCAAGAAGGGGAACAGCGCCGACCTGACCT
CCCCCGAGAAGATCCAGGCCATCATGACCAGCCTCCAGGACTTCAAGATC
GTGCCCATCGACCCCACGAAGAACATCATGGGCATCGAGGTCCCGGAGAC
CCTGGTGCACAAGCTGACCGGGAAGAAGGTGACCTCCAAGAACGGCCAGC
CCATCATCCCCGTCCTCCTGCCGAAGTACATCGGCCTGGACCCCGTGGCC
CCCGGGGACCTCACGATGGTGATCACCCAGGACTGCGACACCTGCCACAG
CCCCGCCAGCCTGCCGGCGGTCATCGAGAAGTGAGGACTAGTGCATCACA
TTTAAAAGCATCTCAGCCTACCATGAGAATAAGAGAAAGAAAATGAAGAT
CAATAGCTTATTCATCTCTTTTTCTTTTTCGTTGGTGTAAAGCCAACACC
CTGTCTAAAAAACATAAATTTCTTTAATCATTTTGCCTCTTTTCTCTGTG
CTTCAATTAATAAAAAATGGAAAGAACCTAGATCTAAAAAAAAAAAAAAA
AAAAAAAAAAAAAAAAAAAAAAAAAAAAAAAAAAAAAAAAAAAAAAAAAT
GCATCCCCCCCCCCCCCCCCCCCCCCCCCCCCCCCAAAGGCTCTTTTCAG
AGCCACCAGAATT

The RNA sequence corresponding to SEQ ID NO. 40 is defined by SEQ ID NO. 48.

The following optimised nucleotide sequence (corresponding to the optimized mRNA sequence according to the invention) according to SEQ ID NO. 41 corresponds to the amino acid sequence according to SEQ ID NO. 8 and refers to the matrix protein VP40 of an Ebolavirus strain EBOV isolated in Sierra Leone in 2014 as described above.

EBOV VP40, Sierra Leone 2014
Optimised nucleotide sequence (SEQ ID NO. 41):
GGGGCGCTGCCTACGGAGGTGGCAGCCATCTCCTTCTCGGCATCAAGCTT
ACCATGCGCCGGGTGATCCTGCCCACCGCCCCGCCCGAGTACATGGAGGC
CATCTACCCCGCGCGCAGCAACTCCACCATCGCCCGGGGCGGGAACAGCA
ACACGGGCTTCCTCACCCCCGAGTCCGTCAACGGGGACACCCCGAGCAAC
CCCCTGCGCCCCATCGCCGACGACACCATCGACCACGCCTCCCACACGCC
CGGCAGCGTGTCCAGCGCCTTCATCCTGGAGGCGATGGTGAACGTCATCT
CCGGGCCGAAGGTGCTCATGAAGCAGATCCCCATCTGGCTGCCCCTGGGC
GTGGCCGACCAGAAGACCTACAGCTTCGACTCCACCACCGCCGCCATCAT
GCTCGCGAGCTACACGATCACCCACTTCGGCAAGGCCACCAACCCCCTGG
TCCGGGTGAACCGCCTGGGGCCGGGCATCCCCGACCACCCCCTCCGGCTG
CTGCGCATCGGGAACCAGGCCTTCCTCCAGGAGTTCGTGCTGCCCCCGGT
CCAGCTGCCCCAGTACTTCACCTTCGACCTCACGGCCCTGAAGCTGATCA
CCCAGCCCCTCCCCGCCGCGACCTGGACCGACGACACGCCGACCGGCTCC
AACGGGGCCCTGCGGCCCGGCATCAGCTTCCACCCCAAGCTGCGCCCCAT
CCTCCTGCCGAACAAGTCCGGCAAGAAGGGGAACAGCGCCGACCTGACCT
CCCCCGAGAAGATCCAGGCCATCATGACCAGCCTCCAGGACTTCAAGATC
GTGCCCATCGACCCCACGAAGAACATCATGGGCATCGAGGTGCCGGAGAC
CCTGGTCCACAAGCTGACCGGGAAGAAGGTGACCTCCAAGAACGGCCAGC
CCATCATCCCCGTGCTCCTGCCGAAGTACATCGGCCTGGACCCCGTCGCC
CCCGGGGACCTCACGATGGTGATCACCCAGGACTGCGACACCTGCCACAG
CCCCGCGAGCCTGCCGGCCGTGGTCGAGAAGTGAGGACTAGTGCATCACA
TTTAAAAGCATCTCAGCCTACCATGAGAATAAGAGAAAGAAAATGAAGAT
CAATAGCTTATTCATCTCTTTTTCTTTTTCGTTGGTGTAAAGCCAACACC
CTGTCTAAAAAACATAAATTTCTTTAATCATTTTGCCTCTTTTCTCTGTG
CTTCAATTAATAAAAAATGGAAAGAACCTAGATCTAAAAAAAAAAAAAAA
AAAAAAAAAAAAAAAAAAAAAAAAAAAAAAAAAAAAAAAAAAAAAAAAAT
GCATCCCCCCCCCCCCCCCCCCCCCCCCCCCCCCCAAAGGCTCTTTTCAG
AGCCACCAGAATT

The RNA sequence corresponding to SEQ ID NO. 41 is defined by SEQ ID NO. 49.

The following optimised nucleotide sequence (corresponding to the optimized mRNA sequence according to the invention) according to SEQ ID NO. 42 corresponds to the amino acid sequence according to SEQ ID NO. 9 and refers to the matrix protein VP40 of a Marburgvirus strain MARV isolated in Angola in 2005 as described above.

MARV VP40, Angola 2005
Optimised nucleotide sequence (SEQ ID NO. 42):
GGGGCGCTGCCTACGGAGGTGGCAGCCATCTCCTTCTCGGCATCAAGCTT
ACCATGGCCAGCTCCAGCAACTACAACACCTACATGCAGTACCTGAACCC
GCCGCCCTACGCCGACCACGGCGCGAACCAGCTCATCCCCGCCGACCAGC
TGTCCAACCAGCAGGGGATCACCCCCAACTACGTGGGCGACCTGAACCTC
GACGACCAGTTCAAGGGGAACGTCTGCCACGCCTTCACGCTGGAGGCCAT
CATCGACATCAGCGCCTACAACGAGCGCACCGTGAAGGGCGTGCCGGCGT
GGCTGCCCCTCGGGATCATGTCCAACTTCGAGTACCCCCTGGCCCACACC
GTCGCCGCCCTGCTCACCGGCAGCTACACGATCACCCAGTTCACCCACAA
CGGCCAGAAGTTCGTGCGGGTGAACCGCCTGGGGACCGGCATCCCCGCGC
ACCCGCTGCGGATGCTCCGCGAGGGGAACCAGGCCTTCATCCAGAACATG
GTCATCCCCCGGAACTTCTCCACGAACCAGTTCACCTACAACCTGACCAA
CCTGGTGCTCAGCGTGCAGAAGCTGCCCGACGACGCCTGGCGCCCCTCCA
AGGACAAGCTGATCGGCAACACCATGCACCCCGCCGTCAGCGTGCACCCC
AACCTCCCGCCCATCGTGCTGCCGACGGTCAAGAAGCAGGCCTACCGGCA
GCACAAGAACCCCAACAACGGGCCCCTGCTCGCGATCTCCGGCATCCTGC
ACCAGCTGCGCGTGGAGAAGGTGCCCGAGAAGACCAGCCTCTTCCGGATC
TCCCTGCCGGCCGACATGTTCAGCGTCAAGGAGGGCATGATGAAGAAGCG
CGGGGAGAACTCCCCCGTGGTGTACTTCCAGGCCCCCGAGAACTTCCCCC
TGAACGGCTTCAACAACCGGCAGGTCGTGCTCGCCTACGCCAACCCGACC
CTGAGCGCGGTGTGAGGACTAGTGCATCACATTTAAAAGCATCTCAGCCT
ACCATGAGAATAAGAGAAAGAAAATGAAGATCAATAGCTTATTCATCTCT
TTTTCTTTTTCGTTGGTGTAAAGCCAACACCCTGTCTAAAAAACATAAAT
TTCTTTAATCATTTTGCCTCTTTTCTCTGTGCTTCAATTAATAAAAAATG
GAAAGAACCTAGATCTAAAAAAAAAAAAAAAAAAAAAAAAAAAAAAAAAA
AAAAAAAAAAAAAAAAAAAAAAAAAAAAAATGCATCCCCCCCCCCCCCCC
CCCCCCCCCCCCCCCCAAAGGCTCTTTTCAGAGCCACCAGAATT

The RNA sequence corresponding to SEQ ID NO. 42 is defined by SEQ ID NO. 50.

The following optimised nucleotide sequence (corresponding to the optimized mRNA sequence according to the invention) according to SEQ ID NO. 43 corresponds to the amino acid sequence according to SEQ ID NO. 13 and refers to the nucleoprotein NP of an Ebolavirus strain EBOV isolated in Zaire in 1976 as described above.

EBOV NP, Zaire 1976
Optimised nucleotide sequence (SEQ ID NO. 43):
GGGGCGCTGCCTACGGAGGTGGCAGCCATCTCCTTCTCGGCATCAAGCTT
ACCATGGACAGCCGCCCCCAGAAGATCTGGATGGCCCCGTCCCTGACCGA
GAGCGACATGGACTACCACAAGATCCTCACCGCCGGCCTGTCCGTGCAGC
AGGGGATCGTCCGGCAGCGCGTGATCCCCGTGTACCAGGTCAACAACCTG
GAGGAGATCTGCCAGCTCATCATCCAGGCGTTCGAGGCCGGCGTGGACTT
CCAGGAGAGCGCCGACTCCTTCCTGCTGATGCTCTGCCTGCACCACGCCT
ACCAGGGGGACTACAAGCTGTTCCTCGAGAGCGGCGCCGTGAAGTACCTG
GAGGGGCACGGCTTCCGGTTCGAGGTCAAGAAGCGCGACGGCGTGAAGCG
GCTGGAGGAGCTCCTGCCCGCGGTGTCCAGCGGGAAGAACATCAAGCGCA
CGCTGGCCGCCATGCCCGAGGAGGAGACCACCGAGGCCAACGCGGGCCAG
TTCCTCTCCTTCGCCAGCCTGTTCCTGCCGAAGCTCGTCGTGGGGGAGAA
GGCCTGCCTGGAGAAGGTGCAGCGGCAGATCCAGGTCCACGCCGAGCAGG
GCCTGATCCAGTACCCCACCGCCTGGCAGTCCGTGGGGCACATGATGGTG
ATCTTCCGCCTCATGCGGACGAACTTCCTGATCAAGTTCCTGCTCATCCA
CCAGGGCATGCACATGGTCGCGGGCCACGACGCCAACGACGCCGTGATCA
GCAACTCCGTGGCCCAGGCCCGCTTCAGCGGGCTGCTGATCGTCAAGACC
GTGCTCGACCACATCCTGCAGAAGACCGAGCGGGGCGTGCGCCTGCACCC
CCTCGCGCGGACCGCCAAGGTCAAGAACGAGGTGAACTCCTTCAAGGCCG
CCCTGAGCTCCCTGGCCAAGCACGGGGAGTACGCGCCCTTCGCCCGCCTC
CTGAACCTGAGCGGCGTGAACAACCTCGAGCACGGCCTGTTCCCGCAGCT
GTCCGCCATCGCCCTCGGGGTCGCCACGGCGCACGGCAGCACCCTGGCCG
GGGTGAACGTCGGCGAGCAGTACCAGCAGCTGCGGGAGGCCGCCACCGAG
GCGGAGAAGCAGCTCCAGCAGTACGCCGAGAGCCGCGAGCTGGACCACCT
GGGGCTCGACGACCAGGAGAAGAAGATCCTGATGAACTTCCACCAGAAGA
AGAACGAGATCTCCTTCCAGCAGACCAACGCCATGGTGACGCTGCGGAAG
GAGCGCCTGGCCAAGCTCACCGAGGCCATCACCGCGGCCAGCCTGCCCAA
GACCTCCGGCCACTACGACGACGACGACGACATCCCCTTCCCCGGCCCGA
TCAACGACGACGACAACCCCGGGCACCAGGACGACGACCCCACGGACAGC
CAGGACACCACCATCCCCGACGTGGTCGTGGACCCGGACGACGGCTCCTA
CGGGGAGTACCAGAGCTACTCCGAGAACGGCATGAACGCCCCCGACGACC
TGGTGCTCTTCGACCTGGACGAGGACGACGAGGACACCAAGCCCGTCCCC
AACCGGAGCACGAAGGGCGGGCAGCAGAAGAACTCCCAGAAGGGCCAGCA
CATCGAGGGGCGCCAGACCCAGAGCCGGCCGATCCAGAACGTGCCCGGCC
CCCACCGCACCATCCACCACGCCTCCGCCCCGCTGACCGACAACGACCGC
CGGAACGAGCCCAGCGGGTCCACGAGCCCCCGCATGCTCACCCCCATCAA
CGAGGAGGCGGACCCCCTGGACGACGCCGACGACGAGACCTCCAGCCTGC
CGCCCCTCGAGTCCGACGACGAGGAGCAGGACCGGGACGGCACCAGCAAC
CGCACGCCCACCGTGGCCCCGCCCGCCCCCGTCTACCGGGACCACTCCGA
GAAGAAGGAGCTGCCCCAGGACGAGCAGCAGGACCAGGACCACACCCAGG
AGGCCCGCAACCAGGACAGCGACAACACCCAGAGCGAGCACTCCTTCGAG
GAGATGTACCGGCACATCCTGCGCAGCCAGGGGCCGTTCGACGCGGTGCT
CTACTACCACATGATGAAGGACGAGCCCGTGGTCTTCTCCACGAGCGACG
GCAAGGAGTACACCTACCCCGACTCCCTGGAGGAGGAGTACCCGCCGTGG
CTGACCGAGAAGGAGGCCATGAACGAGGAGAACCGGTTCGTGACCCTCGA
CGGCCAGCAGTTCTACTGGCCCGTGATGAACCACAAGAACAAGTTCATGG
CCATCCTGCAGCACCACCAGTGAGGACTAGTGCATCACATTTAAAAGCAT
CTCAGCCTACCATGAGAATAAGAGAAAGAAAATGAAGATCAATAGCTTAT
TCATCTCTTTTTCTTTTTCGTTGGTGTAAAGCCAACACCCTGTCTAAAAA
ACATAAATTTCTTTAATCATTTTGCCTCTTTTCTCTGTGCTTCAATTAAT
AAAAAATGGAAAGAACCTAGATCTAAAAAAAAAAAAAAAAAAAAAAAAAA
AAAAAAAAAAAAAAAAAAAAAAAAAAAAAAAAAAAAAATGCATCCCCCCC
CCCCCCCCCCCCCCCCCCCCCCCCAAAGGCTCTTTTCAGAGCCACCAGAA
TT

The RNA sequence corresponding to SEQ ID NO. 43 is defined by SEQ ID NO. 51.

The following optimised nucleotide sequence (corresponding to the optimized mRNA sequence according to the invention) according to SEQ ID NO. 44 corresponds to the amino acid sequence according to SEQ ID NO. 14 and refers to the nucleoprotein NP of an Ebolavirus strain EBOV isolated in Sierra Leone in 2014 as described above.

EBOV NP, Sierra Leone 2014
Optimised nucleotide sequence (SEQ ID NO. 44):
GGGGCGCTGCCTACGGAGGTGGCAGCCATCTCCTTCTCGGCATCAAGCTT
ACCATGGACAGCCGCCCCCAGAAGGTGTGGATGACCCCGTCCCTGACCGA
GAGCGACATGGACTACCACAAGATCCTCACGGCCGGCCTGTCCGTCCAGC
AGGGGATCGTGCGGCAGCGCGTGATCCCCGTCTACCAGGTGAACAACCTG
GAGGAGATCTGCCAGCTCATCATCCAGGCCTTCGAGGCGGGCGTGGACTT
CCAGGAGAGCGCCGACTCCTTCCTGCTGATGCTCTGCCTGCACCACGCCT
ACCAGGGGGACTACAAGCTGTTCCTCGAGAGCGGCGCCGTCAAGTACCTG
GAGGGGCACGGCTTCCGGTTCGAGGTGAAGAAGTGCGACGGCGTGAAGCG
CCTGGAGGAGCTCCTGCCCGCCGTCTCCAGCGGGCGGAACATCAAGCGCA
CCCTGGCGGCCATGCCCGAGGAGGAGACCACCGAGGCCAACGCCGGCCAG
TTCCTCTCCTTCGCGAGCCTGTTCCTGCCGAAGCTCGTGGTGGGGGAGAA
GGCCTGCCTGGAGAAGGTCCAGCGGCAGATCCAGGTGCACGCCGAGCAGG
GCCTGATCCAGTACCCCACGGCCTGGCAGTCCGTGGGGCACATGATGGTC
ATCTTCCGCCTCATGCGGACCAACTTCCTGATCAAGTTCCTGCTCATCCA
CCAGGGCATGCACATGGTGGCCGGCCACGACGCGAACGACGCCGTGATCA
GCAACTCCGTCGCCCAGGCCCGCTTCAGCGGGCTGCTGATCGTGAAGACC
GTGCTCGACCACATCCTGCAGAAGACCGAGCGGGGCGTCCGCCTGCACCC
CCTCGCCCGGACGGCGAAGGTGAAGAACGAGGTGAACTCCTTCAAGGCCG
CCCTGAGCTCCCTGGCCAAGCACGGGGAGTACGCCCCCTTCGCGCGCCTC
CTGAACCTGAGCGGCGTCAACAACCTCGAGCACGGCCTGTTCCCGCAGCT
GTCCGCCATCGCCCTCGGGGTGGCCACCGCCCACGGCAGCACCCTGGCGG
GGGTCAACGTGGGCGAGCAGTACCAGCAGCTGCGGGAGGCCGCCACCGAG
GCCGAGAAGCAGCTCCAGCAGTACGCGGAGAGCCGCGAGCTGGACCACCT
GGGGCTCGACGACCAGGAGAAGAAGATCCTGATGAACTTCCACCAGAAGA
AGAACGAGATCTCCTTCCAGCAGACGAACGCCATGGTGACCCTGCGGAAG
GAGCGCCTGGCCAAGCTCACCGAGGCCATCACCGCCGCGAGCCTGCCCAA
GACGTCCGGCCACTACGACGACGACGACGACATCCCCTTCCCCGGCCCGA
TCAACGACGACGACAACCCCGGGCACCAGGACGACGACCCCACCGACAGC
CAGGACACCACCATCCCCGACGTCGTGGTGGACCCGGACGACGGCGGGTA
CGGCGAGTACCAGTCCTACAGCGAGAACGGGATGTCCGCCCCCGACGACC
TGGTCCTCTTCGACCTGGACGAGGACGACGAGGACACGAAGCCCGTGCCC
AACCGGAGCACCAAGGGCGGCCAGCAGAAGAACTCCCAGAAGGGGCAGCA
CACCGAGGGCCGCCAGACCCAGAGCACGCCGACCCAGAACGTGACCGGGC
CCCGGCGCACCATCCACCACGCCTCCGCCCCGCTGACGGACAACGACCGC
CGGAACGAGCCCAGCGGCTCCACCAGCCCGCGCATGCTCACCCCCATCAA
CGAGGAGGCCGACCCCCTGGACGACGCGGACGACGAGACCTCCAGCCTGC
CCCCGCTCGAGTCCGACGACGAGGAGCAGGACCGGGACGGGACGAGCAAC
CGCACCCCCACCGTCGCCCCGCCCGCCCCCGTGTACCGGGACCACTCCGA
GAAGAAGGAGCTGCCCCAGGACGAGCAGCAGGACCAGGACCACATCCAGG
AGGCCCGCAACCAGGACAGCGACAACACCCAGCCCGAGCACAGCTTCGAG
GAGATGTACCGGCACATCCTGCGCTCCCAGGGCCCGTTCGACGCCGTGCT
CTACTACCACATGATGAAGGACGAGCCCGTCGTGTTCAGCACGTCCGACG
GCAAGGAGTACACCTACCCCGACAGCCTGGAGGAGGAGTACCCGCCGTGG
CTGACCGAGAAGGAGGCGATGAACGACGAGAACCGGTTCGTGACCCTCGA
CGGGCAGCAGTTCTACTGGCCCGTCATGAACCACCGCAACAAGTTCATGG
CCATCCTGCAGCACCACCAGTGAGGACTAGTGCATCACATTTAAAAGCAT
CTCAGCCTACCATGAGAATAAGAGAAAGAAAATGAAGATCAATAGCTTAT
TCATCTCTTTTTCTTTTTCGTTGGTGTAAAGCCAACACCCTGTCTAAAAA
ACATAAATTTCTTTAATCATTTTGCCTCTTTTCTCTGTGCTTCAATTAAT
AAAAAATGGAAAGAACCTAGATCTAAAAAAAAAAAAAAAAAAAAAAAAAA
AAAAAAAAAAAAAAAAAAAAAAAAAAAAAAAAAAAAAATGCATCCCCCCC
CCCCCCCCCCCCCCCCCCCCCCCCAAAGGCTCTTTTCAGAGCCACCAGAA
TT

The RNA sequence corresponding to SEQ ID NO. 44 is defined by SEQ ID NO. 52.

In further specific embodiments, the mRNA sequence according to the invention may further comprise one or more internal ribosome entry site (IRES) sequences or IRES-motifs, which may separate several open reading frames, for example if the inventive mRNA sequence encodes for two or more antigenic peptides or proteins. An IRES-sequence may be particularly helpful if the mRNA is a bi- or multicistronic mRNA. Particularly preferred are IRES sequences according to SEQ ID NO. 28 and SEQ ID NO. 29.

Additionally, the inventive mRNA sequence may be prepared using any method known in the art, including synthetic methods such as e.g. solid phase synthesis, as well as in vitro methods, such as in vitro transcription reactions.

According to one embodiment of the present invention the mRNA sequence comprising a coding region, encoding at least one antigenic peptide or protein of Ebolavirus or Marburgvirus as outlined above or a fragment, variant or derivative thereof may be administered naked without being associated with any further vehicle, transfection or complexation agent for increasing the transfection efficiency and/or the immunostimulatory properties of the inventive mRNA sequence or of further comprised nucleic acid.

In a preferred embodiment, the inventive mRNA sequence may be formulated together with a cationic or polycationic compound and/or with a polymeric carrier. Accordingly, in a further embodiment of the invention it is preferred that the inventive mRNA sequence or any other nucleic acid comprised in the inventive pharmaceutical composition or vaccine is associated with or complexed with a cationic or polycationic compound or a polymeric carrier, optionally in a weight ratio selected from a range of about 6:1 (w/w) to about 0.25:1 (w/w), more preferably from about 5:1 (w/w) to about 0.5:1 (w/w), even more preferably of about 4:1 (w/w) to about 1:1 (w/w) or of about 3:1 (w/w) to about 1:1 (w/w), and most preferably a ratio of about 3:1 (w/w) to about 2:1 (w/w) of mRNA or nucleic acid to cationic or polycationic compound and/or with a polymeric carrier; or optionally in a nitrogen/phosphate ratio of mRNA or nucleic acid to cationic or polycationic compound and/or polymeric carrier in the range of about 0.1-10, preferably in a range of about 0.3-4 or 0.3-1, and most preferably in a range of about 0.5-1 or 0.7-1, and even most preferably in a range of about 0.3-0.9 or 0.5-0.9.

Thereby, the inventive mRNA sequence or any other nucleic acid comprised in the inventive pharmaceutical composition or vaccine can also be associated with a vehicle, transfection or complexation agent for increasing the transfection efficiency and/or the immunostimulatory properties of the inventive mRNA or of optionally comprised further included nucleic acids.

Cationic or polycationic compounds, being particularly preferred agents in this context include protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, pIsl, FGF, Lactoferrin, Transportan, Buforin-2, Bac715-24, SynB, SynB(1), pVEC, hCT-derived peptides, SAP, or histones.

In this context protamine is particularly preferred.

Additionally, preferred cationic or polycationic proteins or peptides may be selected from the following proteins or peptides having the following total formula (III):

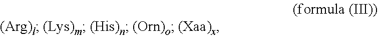

wherein l+m+n+0+x=8-15, and l, m, n or o independently of each other may be any number selected from 0, 1, 2, 3, 4, 5, 6, 7, 8, 9, 10, 11, 12, 13, 14 or 15, provided that the overall content of Arg, Lys, His and Orn represents at least 50% of all amino acids of the oligopeptide; and Xaa may be any amino acid selected from native (=naturally occurring) or non-native amino acids except of Arg, Lys, His or Orn; and x may be any number selected from 0, 1, 2, 3 or 4, provided, that the overall content of Xaa does not exceed 50% of all amino acids of the oligopeptide. Particularly preferred cationic peptides in this context are e.g. Arg₇, Arg₈, Arg₉, H₃R₉, R₉H₃, H₃R₉H₃, YSSR₉SSY, (RKH)₄, Y(RKH)₂R, etc. In this context the disclosure of WO 2009/030481 is incorporated herewith by reference.

Further preferred cationic or polycationic compounds, which can be used as transfection or complexation agent may include cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, DOTAP: dioleoyloxy-3-(trimethylammonio)propane, DC-6-14: O,O-ditetradecanoyl-N-(α-trimethylammonioacetyl)diethanolamine chloride, CLIP1: rac-[(2,3-dioctadecyloxypropyl)(2-hydroxyethyl)]-dimethylammonium chloride, CLIP6: rac-[2(2,3-dihexadecyloxypropyl-oxymethyloxy)ethyl]trimethylammonium, CLIP9: rac-[2(2,3-dihexadecyloxypropyl-oxysuccinyloxy)ethyl]-trimethylammonium, oligofectamine, or cationic or polycationic polymers, e.g. modified polyaminoacids, such as β-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

A polymeric carrier used according to the invention might be a polymeric carrier formed by disulfide-crosslinked cationic components. The disulfide-crosslinked cationic components may be the same or different from each other. The polymeric carrier can also contain further components. It is also particularly preferred that the polymeric carrier used according to the present invention comprises mixtures of cationic peptides, proteins or polymers and optionally further components as defined herein, which are crosslinked by disulfide bonds as described herein. In this context the disclosure of WO 2012/013326 is incorporated herewith by reference.

In this context the cationic components, which form basis for the polymeric carrier by disulfide-crosslinkage, are typically selected from any suitable cationic or polycationic peptide, protein or polymer suitable for this purpose, particular any cationic or polycationic peptide, protein or polymer capable to complex an mRNA or a nucleic acid as defined according to the present invention, and thereby preferably condensing the mRNA or the nucleic acid. The cationic or polycationic peptide, protein or polymer, is preferably a linear molecule, however, branched cationic or polycationic peptides, proteins or polymers may also be used.

Every disulfide-crosslinking cationic or polycationic protein, peptide or polymer of the polymeric carrier, which may be used to complex the inventive mRNA or any further nucleic acid comprised in the inventive pharmaceutical composition or vaccine contains at least one —SH moiety, most preferably at least one cysteine residue or any further chemical group exhibiting an —SH moiety, capable to form a disulfide linkage upon condensation with at least one further cationic or polycationic protein, peptide or polymer as cationic component of the polymeric carrier as mentioned herein.

As defined above, the polymeric carrier, which may be used to complex the inventive mRNA sequence or any further nucleic acid comprised in the inventive pharmaceutical composition or vaccine may be formed by disulfide-crosslinked cationic (or polycationic) components.

Preferably, such cationic or polycationic peptides or proteins or polymers of the polymeric carrier, which comprise or are additionally modified to comprise at least one —SH moiety, are selected from, proteins, peptides and polymers as defined above for complexation agent.

In a further particular embodiment, the polymeric carrier which may be used to complex the inventive mRNA sequence or any further nucleic acid comprised in the inventive pharmaceutical composition or vaccine may be selected from a polymeric carrier molecule according to generic formula (IV):

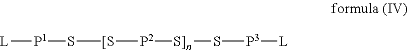

wherein,
P¹ and P³ are different or identical to each other and represent a linear or branched hydrophilic polymer chain, each P¹ and P³ exhibiting at least one —SH-moiety, capable to form a disulfide linkage upon condensation with component P², or alternatively with (AA), (AA)ₓ, or [(AA)ₓ]_{z} if such components are used as a linker between P¹ and P² or P³ and P²) and/or with further components (e.g. (AA), (AA)ₓ, [(AA)ₓ]_{z} or L), the linear or branched hydrophilic polymer chain selected independent from each other from polyethylene glycol (PEG), poly-N-(2-hydroxypropyl)methacrylamide, poly-2-(methacryloyloxy)ethyl phosphorylcholines, poly(hydroxyalkyl L-asparagine), poly(2-phosphorylcholine), hydroxyethylstarch or (methacryloyloxy)ethyl poly(hydroxyalkyl L-glutamine), wherein the hydrophilic polymer chain exhibits a molecular weight of about 1 kDa to about 100 kDa, preferably of about 2 kDa to about 25 kDa; or more preferably of about 2 kDa to about 10 kDa, e.g. about 5 kDa to about 25 kDa or 5 kDa to about 10 kDa;
P² is a cationic or polycationic peptide or protein, e.g. as defined above for the polymeric carrier formed by disulfide-crosslinked cationic components, and preferably having a length of about 3 to about 100 amino acids, more preferably having a length of about 3 to about 50 amino acids, even more preferably having a length of about 3 to about 25 amino acids, e.g. a length of about 3 to 10, 5 to 15, 10 to 20 or 15 to 25 amino acids, more preferably a length of about 5 to about 20 and even more preferably a length of about 10 to about 20; or
   is a cationic or polycationic polymer, e.g. as defined above for the polymeric carrier formed by disulfide-crosslinked cationic components, typically having a molecular weight of about 0.5 kDa to about 30 kDa, including a molecular weight of about 1 kDa to about 20 kDa, even more preferably of about 1.5 kDa to about 10 kDa, or having a molecular weight of about 0.5 kDa to about 100 kDa, including a molecular weight of about 10 kDa to about 50 kDa, even more preferably of about 10 kDa to about 30 kDa;
   each P² exhibiting at least two —SH-moieties, capable to form a disulfide linkage upon condensation with further components P² or component(s) P¹ and/or P³ or alternatively with further components (e.g. (AA), (AA)ₓ, or [(AA)ₓ]_{z});
—S—S— is a (reversible) disulfide bond (the brackets are omitted for better readability), wherein S preferably represents sulphur or a —SH carrying moiety, which has formed a (reversible) disulfide bond. The (reversible) disulfide bond is preferably formed by condensation of —SH-moieties of either components P¹ and P², P² and P², or P² and P³, or optionally of further components as defined herein (e.g. L, (AA), (AA)ₓ, [(AA)ₓ]_{z}, etc); The —SH-moiety may be part of the structure of these components or added by a modification as defined below;
L is an optional ligand, which may be present or not, and may be selected independent from the other from RGD, Transferrin, Folate, a signal peptide or signal sequence, a localization signal or sequence, a nuclear localization signal or sequence (NLS), an antibody, a cell penetrating peptide, (e.g. TAT or KALA), a ligand of a receptor (e.g. cytokines, hormones, growth factors etc), small molecules (e.g. carbohydrates like mannose or galactose or synthetic ligands), small molecule agonists, inhibitors or antagonists of receptors (e.g. RGD peptidomimetic analogues), or any further protein as defined herein, etc.;
n is an integer, typically selected from a range of about 1 to 50, preferably from a range of about 1, 2 or 3 to 30, more preferably from a range of about 1, 2, 3, 4, or 5 to 25, or a range of about 1, 2, 3, 4, or 5 to 20, or a range of about 1, 2, 3, 4, or 5 to 15, or a range of about 1, 2, 3, 4, or 5 to 10, including e.g. a range of about 4 to 9, 4 to 10, 3 to 20, 4 to 20, 5 to 20, or 10 to 20, or a range of about 3 to 15, 4 to 15, 5 to 15, or 10 to 15, or a range of about 6 to 11 or 7 to 10. Most preferably, n is in a range of about 1, 2, 3, 4, or 5 to 10, more preferably in a range of about 1, 2, 3, or 4 to 9, in a range of about 1, 2, 3, or 4 to 8, or in a range of about 1, 2, or 3 to 7.

In this context the disclosure of WO 2011/026641 is incorporated herewith by reference. Each of hydrophilic polymers P¹ and P³ typically exhibits at least one —SH-moiety, wherein the at least one —SH-moiety is capable to form a disulfide linkage upon reaction with component P² or with component (AA) or (AA)ₓ, if used as linker between P¹ and P² or P³ and P² as defined below and optionally with a further component, e.g. L and/or (AA) or (AA)ₓ, e.g. if two or more —SH-moieties are contained. The following subformulae “P¹—S—S—P²” and “P²—S—S—P³” within generic formula (V) above (the brackets are omitted for better readability), wherein any of S, P¹ and P³ are as defined herein, typically represent a situation, wherein one-SH-moiety of hydrophilic polymers P¹ and P³ was condensed with one —SH-moiety of component P² of generic formula (V) above, wherein both sulphurs of these —SH-moieties form a disulfide bond —S—S— as defined herein in formula (V). These —SH-moieties are typically provided by each of the hydrophilic polymers P¹ and P³, e.g. via an internal cysteine or any further (modified) amino acid or compound which carries a —SH moiety. Accordingly, the subformulae “P¹—S—S—P²” and “P²—S—S—P³” may also be written as “P¹-Cys-Cys-P²” and “P²-Cys-Cys-P³”, if the —SH— moiety is provided by a cysteine, wherein the term Cys-Cys represents two cysteines coupled via a disulfide bond, not via a peptide bond. In this case, the term “—S—S—” in these formulae may also be written as “—S-Cys”, as “-Cys-S” or as “-Cys-Cys-”. In this context, the term “-Cys-Cys-” does not represent a peptide bond but a linkage of two cysteines via their —SH-moieties to form a disulfide bond. Accordingly, the term “-Cys-Cys-” also may be understood generally as “-(Cys-S)—(S-Cys)-”, wherein in this specific case S indicates the sulphur of the —SH-moiety of cysteine. Likewise, the terms “—S-Cys” and “—Cys-S” indicate a disulfide bond between a —SH containing moiety and a cysteine, which may also be written as “—S—(S-Cys)” and “-(Cys-S)—S”. Alternatively, the hydrophilic polymers P¹ and P³ may be modified with a —SH moiety, preferably via a chemical reaction with a compound carrying a —SH moiety, such that each of the hydrophilic polymers P¹ and P³ carries at least one such —SH moiety. Such a compound carrying a —SH moiety may be e.g. an (additional) cysteine or any further (modified) amino acid, which carries a —SH moiety. Such a compound may also be any non-amino compound or moiety, which contains or allows to introduce a —SH moiety into hydrophilic polymers P¹ and P³ as defined herein. Such non-amino compounds may be attached to the hydrophilic polymers P¹ and P³ of formula (VI) of the polymeric carrier according to the present invention via chemical reactions or binding of compounds, e.g. by binding of a 3-thio propionic acid or thioimolane, by amide formation (e.g. carboxylic acids, sulphonic acids, amines, etc), by Michael addition (e.g maleinimide moieties, α, β unsatured carbonyls, etc), by click chemistry (e.g. azides or alkines), by alkene/alkine methatesis (e.g. alkenes or alkines), imine or hydrozone formation (aldehydes or ketons, hydrazins, hydroxylamins, amines), complexation reactions (avidin, biotin, protein G) or components which allow Sn-type substitution reactions (e.g halogenalkans, thiols, alcohols, amines, hydrazines, hydrazides, sulphonic acid esters, oxyphosphonium salts) or other chemical moieties which can be utilized in the attachment of further components. A particularly preferred PEG derivate in this context is alpha-Methoxy-omega-mercapto poly(ethylene glycol). In each case, the SH-moiety, e.g. of a cysteine or of any further (modified) amino acid or compound, may be present at the terminal ends or internally at any position of hydrophilic polymers P¹ and P³. As defined herein, each of hydrophilic polymers P¹ and P³ typically exhibits at least one —SH-moiety preferably at one terminal end, but may also contain two or even more —SH-moieties, which may be used to additionally attach further components as defined herein, preferably further functional peptides or proteins e.g. a ligand, an amino acid component (AA) or (AA)ₓ, antibodies, cell penetrating peptides or enhancer peptides (e.g. TAT, KALA), etc.

In this context it is particularly preferred that the inventive mRNA sequence is complexed at least partially with a cationic or polycationic compound and/or a polymeric carrier, preferably cationic proteins or peptides. In this context the disclosure of WO 2010/037539 and WO 2012/113513 is incorporated herewith by reference. Partially means that only a part of the inventive mRNA sequence is complexed with a cationic compound and that the rest of the inventive mRNA sequence is (comprised in the inventive pharmaceutical composition or vaccine) in uncomplexed form (“free”). Preferably the ratio of complexed mRNA to:free mRNA (in the inventive pharmaceutical composition or vaccine) is selected from a range of about 5:1 (w/w) to about 1:10 (w/w), more preferably from a range of about 4:1 (w/w) to about 1:8 (w/w), even more preferably from a range of about 3:1 (w/w) to about 1:5 (w/w) or 1:3 (w/w), and most preferably the ratio of complexed mRNA to free mRNA in the inventive pharmaceutical composition or vaccine is selected from a ratio of about 1:1 (w/w).

The complexed mRNA in the inventive pharmaceutical composition or vaccine, is preferably prepared according to a first step by complexing the inventive mRNA sequence with a cationic or polycationic compound and/or with a polymeric carrier, preferably as defined herein, in a specific ratio to form a stable complex. In this context, it is highly preferable, that no free cationic or polycationic compound or polymeric carrier or only a negligibly small amount thereof remains in the component of the complexed mRNA after complexing the mRNA. Accordingly, the ratio of the mRNA and the cationic or polycationic compound and/or the polymeric carrier in the component of the complexed mRNA is typically selected in a range that the mRNA is entirely complexed and no free cationic or polycationic compound or polymeric carrier or only a negligibly small amount thereof remains in the composition.

Preferably the ratio of the mRNA to the cationic or polycationic compound and/or the polymeric carrier, preferably as defined herein, is selected from a range of about 6:1 (w/w) to about 0.25:1 (w/w), more preferably from about 5:1 (w/w) to about 0.5:1 (w/w), even more preferably of about 4:1 (w/w) to about 1:1 (w/w) or of about 3:1 (w/w) to about 1:1 (w/w), and most preferably a ratio of about 3:1 (w/w) to about 2:1 (w/w). Alternatively, the ratio of the mRNA to the cationic or polycationic compound and/or the polymeric carrier, preferably as defined herein, in the component of the complexed mRNA, may also be calculated on the basis of the nitrogen/phosphate ratio (N/P-ratio) of the entire complex. In the context of the present invention, an N/P-ratio is preferably in the range of about 0.1-10, preferably in a range of about 0.3-4 and most preferably in a range of about 0.5-2 or 0.7-2 regarding the ratio of mRNA:cationic or polycationic compound and/or polymeric carrier, preferably as defined herein, in the complex, and most preferably in a range of about 0.7-1.5, 0.5-1 or 0.7-1, and even most preferably in a range of about 0.3-0.9 or 0.5-0.9, preferably provided that the cationic or polycationic compound in the complex is a cationic or polycationic cationic or polycationic protein or peptide and/or the polymeric carrier as defined above. In this specific embodiment the complexed mRNA is also encompassed in the term “adjuvant component”.

In certain embodiments of the invention, the mRNA as defined herein may also be replaced by another nucleic acid molecule having the respective structural characteristics and/or functional properties as defined herein. Exemplary nucleic acids envisaged in the ambit of the invention include, but are not limited to, any type of ribonucleic acid (RNA), deoxyribonucleic acid (DNA), threose nucleic acid (TNA), glycol nucleic acid (GNA), peptide nucleic acid (PNA), locked nucleic acids (LNA, including LNA having a β-D-ribo configuration, a-LNA having an α-L-ribo configuration (a diastereomer of LNA), 2′-amino-LNA having a 2′-amino functionalization, and 2′-amino-a-LNA having a 2′-amino functionalization), ethylene nucleic acids (ENA), cyclohexenyl nucleic acids (CeNA) or hybrids or combinations thereof. In particular, the present invention comprises any DNA molecule (such as a DNA vector) encoding the inventive mRNA as described herein.

In a further aspect the invention provides for a composition comprising a plurality or more than one, preferably 2 to 10, more preferably 2 to 5, most preferably 2 to 4 of the inventive mRNA sequences as defined herein. These inventive compositions comprise more than one inventive mRNA sequences, preferably encoding different peptides or proteins which comprise preferably different pathogenic antigens or fragments, variants or derivatives thereof. Particularly preferred in this context is that at least one mRNA sequence encodes at least one antigenic peptide or protein derived from glycoprotein (GP) of a virus of the genus Ebolavirus or Marburgvirus and that at least one mRNA sequence encodes at least one antigenic peptide or protein derived from another antigen of a virus of the genus Ebolavirus or Marburgvirus, particularly of matrix protein 40 (VP40) and/or nucleoprotein (NP).

Accordingly, in a further particular preferred aspect, the present invention also provides a pharmaceutical composition, comprising at least one inventive mRNA sequence as defined herein or an inventive composition comprising a plurality of inventive mRNA sequences as defined herein and optionally a pharmaceutically acceptable carrier and/or vehicle.

As a first ingredient, the inventive pharmaceutical composition comprises at least one inventive mRNA sequence as defined herein.

As a second ingredient the inventive pharmaceutical composition may optional comprise at least one additional pharmaceutically active component. A pharmaceutically active component in this connection is a compound that has a therapeutic effect to heal, ameliorate or prevent a particular indication or disease as mentioned herein, preferably Ebolavirus or Marburgvirus disease or infections. Such compounds include, without implying any limitation, peptides or proteins, preferably as defined herein, nucleic acids, preferably as defined herein, (therapeutically active) low molecular weight organic or inorganic compounds (molecular weight less than 5000, preferably less than 1000), sugars, antigens or antibodies, preferably as defined herein, therapeutic agents already known in the prior art, antigenic cells, antigenic cellular fragments, cellular fractions; cell wall components (e.g. polysaccharides), modified, attenuated or de-activated (e.g. chemically or by irradiation) pathogens (virus, bacteria etc.), adjuvants, preferably as defined herein, etc.

The inventive pharmaceutical composition may be administered orally, parenterally, by inhalation spray, topically, rectally, nasally, buccally, vaginally or via an implanted reservoir. The term parenteral as used herein includes subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, and sublingual injection or infusion techniques.

Particularly preferred is intradermal and intramuscular injection. Sterile injectable forms of the inventive pharmaceutical compositions may be aqueous or oleaginous suspension. These suspensions may be formulated according to techniques known in the art using suitable dispersing or wetting agents and suspending agents.

Preferably, the inventive pharmaceutical composition may be administered by conventional needle injection or needle-free jet injection. In a preferred embodiment the inventive pharmaceutical composition may be administered by jet injection as defined herein, preferably intramuscularly or intradermally, more preferably intradermally.

According to a specific embodiment, the inventive pharmaceutical composition may comprise an adjuvant. In this context, an adjuvant may be understood as any compound, which is suitable to initiate or increase an immune response of the innate immune system, i.e. a non-specific immune response. With other words, when administered, the inventive pharmaceutical composition preferably elicits an innate immune response due to the adjuvant, optionally contained therein. Preferably, such an adjuvant may be selected from an adjuvant known to a skilled person and suitable for the present case, i.e. supporting the induction of an innate immune response in a mammal, e.g. an adjuvant protein as defined above or an adjuvant as defined in the following.

Particularly preferred as adjuvants suitable for depot and delivery are cationic or polycationic compounds as defined above for the inventive mRNA sequence as vehicle, transfection or complexation agent.

Furthermore, the inventive pharmaceutical composition may comprise one or more additional adjuvants which are suitable to initiate or increase an immune response of the innate immune system, i.e. a non-specific immune response, particularly by binding to pathogen-associated molecular patterns (PAMPs). With other words, when administered, the pharmaceutical composition or vaccine preferably elicits an innate immune response due to the adjuvant, optionally contained therein. Preferably, such an adjuvant may be selected from an adjuvant known to a skilled person and suitable for the present case, i.e. supporting the induction of an innate immune response in a mammal, e.g. an adjuvant protein as defined above or an adjuvant as defined in the following. According to one embodiment such an adjuvant may be selected from an adjuvant as defined above.

Also such an adjuvant may be selected from any adjuvant known to a skilled person and suitable for the present case, i.e. supporting the induction of an innate immune response in a mammal and/or suitable for depot and delivery of the components of the inventive pharmaceutical composition or vaccine. Preferred as adjuvants suitable for depot and delivery are cationic or polycationic compounds as defined above. Likewise, the adjuvant may be selected from the group consisting of, e.g., cationic or polycationic compounds as defined above, from chitosan, TDM, MDP, muramyl dipeptide, pluronics, alum solution, aluminium hydroxide, ADJUMER™ (polyphosphazene); aluminium phosphate gel; glucans from algae; algammulin; aluminium hydroxide gel (alum); highly protein-adsorbing aluminium hydroxide gel; low viscosity aluminium hydroxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005™ ((N-(2-deoxy-2-L-leucylaminob-D-glucopyranosyl)-N-octadecyl-dodecanoyl-amide hydroacetate); CALCITRIOL™ (1-alpha, 25-dihydroxy-vitamin D3); calcium phosphate gel; CAP™ (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of: i) N-acetylglucosaminyl-(P1-4)-N-acetylmuramyl-L-alanyl-D35 glutamine (GMDP), ii) dimethyldioctadecylammonium chloride (DDA), iii) zinc-L-proline salt complex (ZnPro-8); GM-CSF); GMDP (N-acetylglucosaminyl-(b1-4)-N-acetylmuramyl-L47 alanyl-D-isoglutamine); imiquimod (1-(2-methypropyl)-1H-imidazo[4,5-c]quinoline-4-amine); ImmTher™ (N-acetylglucosaminyl-N-acetylmuramyl-L-Ala-D-isoGlu-L-Ala-glycerol dipalmitate); DRVs (immunoliposomes prepared from dehydration-rehydration vesicles); interferongamma; interleukin-1beta; interleukin-2; interleukin-7; interleukin-12; ISCOMS™; ISCOPREP 7.0.3.™; liposomes; LOXORIBINE™ (7-allyl-8-oxoguanosine); LT 5 oral adjuvant (E. coli labile enterotoxin-protoxin); microspheres and microparticles of any composition; MF59™; (squalenewater emulsion); MONTANIDE ISA 51™ (purified incomplete Freund's adjuvant); MONTANIDE ISA 720™ (metabolisable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt); MURAMETIDE™ (Nac-Mur-L-Ala-D-Gln-OCH3); MURAPALMITINE™ and DMURAPALMITINE™ (Nac-Mur-L-Thr-D-isoGln-sn-glyceroldipalmitoyl); NAGO (neuraminidase-galactose oxidase); nanospheres or nanoparticles of any composition; NISVs (non-ionic surfactant vesicles); PLEURANT (β-glucan); PLGA, PGA and PLA (homo- and co-polymers of lactic acid and glycolic acid; microspheres/nanospheres); PLURONIC L121™ PMMA (polymethylmethacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, AL); STIMULON™ (QS-21); Quil-A (Quil-A saponin); S-28463 (4-amino-otec-dimethyl-2-ethoxymethyl-1H-imidazo[4,5-c]quinoline-1-ethanol); SAF-1™ (“Syntex adjuvant formulation”); Sendai proteoliposomes and Sendai containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid® (N-acetylglucosaminyl-N-acetylmuramyl-L-Ala-D-isoGlu-L-Aladipalmitoxypropylamide); Theronyl-MDP (Termurtide™ or [thr 1]-MDP; N-acetylmuramyl-Lthreonyl-D-isoglutamine); Ty particles (Ty-VLPs or virus-like particles); Walter-Reed liposomes (liposomes containing lipid A adsorbed on aluminium hydroxide), and lipopeptides, including Pam3Cys, in particular aluminium salts, such as Adju-phos, Alhydrogel, Rehydragel; emulsions, including CFA, SAF, IFA, MF59, Provax, TiterMax, Montanide, Vaxfectin; copolymers, including Optivax (CRL1005), L121, Poloaxmer4010), etc.; liposomes, including Stealth, cochleates, including BIORAL; plant derived adjuvants, including QS21, Quil A, Iscomatrix, ISCOM; adjuvants suitable for costimulation including Tomatine, biopolymers, including PLG, PMM, Inulin, microbe derived adjuvants, including Romurtide, DETOX, MPL, CWS, Mannose, CpG nucleic acid sequences, CpG7909, ligands of human TLR 1-10, ligands of murine TLR 1-13, ISS-1018, 35 IC31, Imidazoquinolines, Ampligen, Ribi529, IMOxine, IRIVs, VLPs, cholera toxin, heat-labile toxin, Pam3Cys, Flagellin, GPI anchor, LNFPIII/Lewis X, antimicrobial peptides, UC-1V150, RSV fusion protein, cdiGMP; and adjuvants suitable as antagonists including CGRP neuropeptide.

Particularly preferred, an adjuvant may be selected from adjuvants, which support induction of a Th1-immune response or maturation of naïve T-cells, such as GM-CSF, IL-12, IFNg, any immunostimulatory nucleic acid as defined above, preferably an immunostimulatory RNA, CpG DNA, etc.

In a further preferred embodiment it is also possible that the inventive pharmaceutical composition contains besides the antigen-providing mRNA further components which are selected from the group comprising: further antigens or further antigen-providing nucleic acids; a further immunotherapeutic agent; one or more auxiliary substances; or any further compound, which is known to be immunostimulating due to its binding affinity (as ligands) to human Toll-like receptors; and/or an adjuvant nucleic acid, preferably an immunostimulatory RNA (isRNA).

The inventive pharmaceutical composition can additionally contain one or more auxiliary substances in order to increase its immunogenicity or immunostimulatory capacity, if desired. A synergistic action of the inventive mRNA sequence as defined herein and of an auxiliary substance, which may be optionally contained in the inventive pharmaceutical composition, is preferably achieved thereby. Depending on the various types of auxiliary substances, various mechanisms can come into consideration in this respect. For example, compounds that permit the maturation of dendritic cells (DCs), for example lipopolysaccharides, TNF-alpha or CD40 ligand, form a first class of suitable auxiliary substances. In general, it is possible to use as auxiliary substance any agent that influences the immune system in the manner of a “danger signal” (LPS, GP96, etc.) or cytokines, such as GM-CFS, which allow an immune response to be enhanced and/or influenced in a targeted manner. Particularly preferred auxiliary substances are cytokines, such as monokines, lymphokines, interleukins or chemokines, that further promote the innate immune response, such as IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta or TNF-alpha, growth factors, such as hGH.

Further additives which may be included in the inventive pharmaceutical composition are emulsifiers, such as, for example, Tween3; wetting agents, such as, for example, sodium lauryl sulfate; colouring agents; taste-imparting agents, pharmaceutical carriers; tablet-forming agents; stabilizers; antioxidants; preservatives.

The inventive pharmaceutical composition can also additionally contain any further compound, which is known to be immunostimulating due to its binding affinity (as ligands) to human Toll-like receptors TLR1, TLR2, TLR3, TLR4, TLR5, TLR6, TLR7, TLR8, TLR9, TLR10, or due to its binding affinity (as ligands) to murine Toll-like receptors TLR1, TLR2, TLR3, TLR4, TLR5, TLR6, TLR7, TLR8, TLR9, TLR10, TLR11, TLR12 or TLR13.

In this context it is particularly preferred that the optionally comprised adjuvant component comprises the same inventive mRNA sequence as comprised in the inventive pharmaceutical composition as antigen-providing mRNA e.g. mRNA coding for an antigenic peptide or protein of Ebolavirus or Marburgvirus or fragments, variants or derivatives thereof.

Despite, the inventive pharmaceutical composition may comprise further components for facilitating administration and uptake of components of the pharmaceutical composition. Such further components may be an appropriate carrier or vehicle, additional adjuvants for supporting any immune response, antibacterial and/or antiviral agents.

Accordingly, in a further embodiment, the inventive pharmaceutical composition furthermore comprises a pharmaceutically acceptable carrier and/or vehicle.

Such a pharmaceutically acceptable carrier typically includes the liquid or non-liquid basis of a composition comprising the components of the inventive pharmaceutical composition. If the composition is provided in liquid form, the carrier will typically be pyrogen-free water; isotonic saline or buffered (aqueous) solutions, e.g. phosphate, citrate etc. buffered solutions. The injection buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of cells due to osmosis or other concentration effects. Reference media are e.g. liquids occurring in “in vivo” methods, such as blood, lymph, cytosolic liquids, or other body liquids, or e.g. liquids, which may be used as reference media in “in vitro” methods, such as common buffers or liquids. Such common buffers or liquids are known to a skilled person. Ringer-Lactate solution is particularly preferred as a liquid basis.

However, one or more compatible solid or liquid fillers or diluents or encapsulating compounds, which are suitable for administration to a patient to be treated, may be used as well for the pharmaceutical composition according to the invention. The term “compatible” as used here means that these constituents of the inventive pharmaceutical composition are capable of being mixed with the components of the inventive pharmaceutical composition in such a manner that no interaction occurs which would substantially reduce the pharmaceutical effectiveness of the pharmaceutical composition under typical use conditions.

A further component of the inventive pharmaceutical composition may be an immunotherapeutic agent that can be selected from immunoglobulins, preferably IgGs, monoclonal or polyclonal antibodies, polyclonal serum or sera, etc, most preferably immunoglobulins directed against Ebolavirus or Marburgvirus. Preferably, such a further immunotherapeutic agent may be provided as a peptide/protein or may be encoded by a nucleic acid, preferably by a DNA or an RNA, more preferably an mRNA. Such an immunotherapeutic agent allows providing passive vaccination additional to active vaccination triggered by the inventive antigen-providing mRNA.

Furthermore, in a specific embodiment, additionally to the antigen-providing mRNA further antigens can be included in the inventive pharmaceutical composition and are typically substances such as cells, cell lysates, viruses, attenuated viruses, inactivated viruses, proteins, peptides, nucleic acids or other bio- or macromolecules or fragments thereof. Preferably, antigens may be proteins and peptides or fragments thereof, such as epitopes of those proteins or peptides, preferably having 5 to 15, more preferably 6 to 9, amino acids. Particularly, said proteins, peptides or epitopes may be derived from glycoprotein (GP) and/or matrix protein 40 (VP40) and/or nucleoprotein (NP) of a virus of the genus Ebolavirus or Marburgvirus or from fragments, variants or derivatives thereof. Further, antigens may also comprise any other biomolecule, e.g., lipids, carbohydrates, etc. Preferably, the antigen is a protein or (poly-) peptide antigen, a nucleic acid, a nucleic acid encoding a protein or (poly-) peptide antigen, a polysaccharide antigen, a polysaccharide conjugate antigen, a lipid antigen, a glycolipid antigen, a carbohydrate antigen, a bacterium, a cell (vaccine), or killed or attenuated viruses. Particularly preferred in this context is the addition of Ebolavirus or Marburgvirus vaccines comprising inactivated virus.

The inventive pharmaceutical composition or vaccine as defined herein may furthermore comprise further additives or additional compounds. Further additives which may be included in the pharmaceutical composition are emulsifiers, such as, for example, Tween3; wetting agents, such as, for example, sodium lauryl sulfate; colouring agents; taste-imparting agents, pharmaceutical carriers; tablet-forming agents; stabilizers; antioxidants; preservatives, RNase inhibitors and/or an anti-bacterial agent or an anti-viral agent. Additionally the inventive pharmaceutical composition may comprise small interfering RNA (siRNA) directed against genes of Ebolavirus or Marburvirus, e.g. siRNA directed against the gene encoding glycoprotein (GP) or matrix protein 40 (VP40) or the nucleoprotein (NP) of a virus of the genus Ebolavirus or Marburgvirus.

The inventive pharmaceutical composition typically comprises a “safe and effective amount” of the components of the inventive pharmaceutical composition, particularly of the inventive mRNA sequence(s) as defined herein. As used herein, a “safe and effective amount” means an amount of the inventive mRNA sequence(s) as defined herein as such that is sufficient to significantly induce a positive modification of a disease or disorder or to prevent a disease, preferably Ebolavirus or Marburgvirus disease as defined herein. At the same time, however, a “safe and effective amount” is small enough to avoid serious side-effects and to permit a sensible relationship between advantage and risk. The determination of these limits typically lies within the scope of sensible medical judgment.

The inventive pharmaceutical composition may be used for human and also for veterinary medical purposes, preferably for human medical purposes, as a pharmaceutical composition in general or as a vaccine.

According to another particularly preferred aspect, the inventive pharmaceutical composition (or the inventive mRNA sequence as defined herein or the inventive composition comprising a plurality of inventive mRNA sequences as defined herein) may be provided or used as a vaccine. Typically, such a vaccine is as defined above for pharmaceutical compositions. Additionally, such a vaccine typically contains the inventive mRNA sequence as defined herein or the inventive composition comprising a plurality of inventive mRNA sequences as defined herein.

The inventive vaccine may also comprise a pharmaceutically acceptable carrier, adjuvant, and/or vehicle as defined herein for the inventive pharmaceutical composition. In the specific context of the inventive vaccine, the choice of a pharmaceutically acceptable carrier is determined in principle by the manner in which the inventive vaccine is administered. The inventive vaccine can be administered, for example, systemically or locally. Routes for systemic administration in general include, for example, transdermal, oral, parenteral routes, including subcutaneous, intravenous, intramuscular, intraarterial, intradermal and intraperitoneal injections and/or intranasal administration routes. Routes for local administration in general include, for example, topical administration routes but also intradermal, transdermal, subcutaneous, or intramuscular injections or intralesional, intracranial, intrapulmonal, intracardial, and sublingual injections. More preferably, vaccines may be administered by an intradermal, subcutaneous, or intramuscular route. Inventive vaccines are therefore preferably formulated in liquid (or sometimes in solid) form. Preferably, the inventive vaccine may be administered by conventional needle injection or needle-free jet injection. In a preferred embodiment the inventive vaccine may be administered by jet injection as defined herein, preferably intramuscularly or intradermally, more preferably intradermally. Particular approaches, methods and features of the administration of an mRNA comprising composition which may be incorporated as certain further embodiments of the present invention are disclosed in WO2015/024667, the description of which is incorporated herein by reference.

The inventive vaccine can additionally contain one or more auxiliary substances in order to increase its immunogenicity or immunostimulatory capacity, if desired. Particularly preferred are adjuvants as auxiliary substances or additives as defined for the pharmaceutical composition.

In a further aspect, the invention is directed to a kit or kit of parts comprising the components of the inventive mRNA sequence, the inventive composition comprising a plurality of inventive mRNA sequences, the inventive pharmaceutical composition or vaccine and optionally technical instructions with information on the administration and dosage of the components.

Beside the components of the inventive mRNA sequence, the inventive composition comprising a plurality of inventive mRNA sequences, the inventive pharmaceutical composition or vaccine the kit may additionally contain a pharmaceutically acceptable vehicle, an adjuvant and at least one further component as defined herein, as well as means for administration and technical instructions. The components of the inventive mRNA sequence, the inventive composition comprising a plurality of inventive mRNA sequences, the inventive pharmaceutical composition or vaccine and e.g. the adjuvant may be provided in lyophilized form. In a preferred embodiment, prior to use of the kit for vaccination, the provided vehicle is than added to the lyophilized components in a predetermined amount as written e.g. in the provided technical instructions. By doing so the inventive mRNA sequence, the inventive composition comprising a plurality of inventive mRNA sequences, the inventive pharmaceutical composition or vaccine, according to the above described aspects of the invention is provided that can afterwards be used in a method as described above, also.

The present invention furthermore provides several applications and uses of the inventive mRNA sequence as defined herein, of the inventive composition comprising a plurality of inventive mRNA sequences as defined herein, of the inventive pharmaceutical composition, of the inventive vaccine, all comprising the inventive mRNA sequence as defined herein or of kits comprising same.

In a further aspect, the invention provides an mRNA sequence encoding at least one antigenic peptide or protein of Ebolavirus or Marburgvirus as outlined above, or a fragment, variant or derivative thereof, and a composition, a pharmaceutical composition, a vaccine and a kit, all comprising the mRNA sequence for use in a method of prophylactic (preexposure prophylaxis or post-exposure prophylaxis) and/or therapeutic treatment of Ebolavirus or Marburgvirus infections. Consequently, in a further aspect, the present invention is directed to the first medical use of the inventive mRNA sequence, the inventive composition comprising a plurality of inventive mRNA sequences, the inventive pharmaceutical composition, the inventive vaccine, and the inventive kit as defined herein as a medicament. Particularly, the invention provides the use of an mRNA sequence encoding at least one antigenic peptide or protein of Ebolavirus or Marburgvirus, or a fragment, variant or derivative thereof as defined above for the preparation of a medicament.

According to another aspect, the present invention is directed to the second medical use of the mRNA sequence encoding at least one antigenic peptide or protein of Ebolavirus or Marburgvirus, or a fragment, variant or derivative thereof, as defined herein, optionally in form of a composition comprising a plurality of inventive mRNA sequences, a pharmaceutical composition or vaccine, kit or kit of parts, for the treatment of Ebolavirus or Marburgvirus infections as defined herein. Particularly, the mRNA sequence encoding at least one antigenic peptide or protein of Ebolavirus or Marburgvirus, or a fragment, variant or derivative thereof to be used in a method as said above is a mRNA sequence formulated together with a pharmaceutically acceptable vehicle and an optionally additional adjuvant and an optionally additional further component as defined above e.g. a further antigen or a Ebolavirus or Marburgvirus disease immune globuline.

In this context the mRNA sequence used for post-exposure treatment of Ebolavirus or Marburgvirus infections according to the invention can be combined with administration of Ebolavirus or Marburgvirus disease immune globuline.

The inventive mRNA sequence may alternatively be provided such that it is administered for preventing or treating Ebolavirus or Marburgvirus infections by several doses, each dose containing the inventive mRNA sequence encoding at least one antigenic peptide or protein of a Ebolavirus or Marburgvirus, or a fragment, variant or derivative thereof as defined above, e.g. the first dose containing at least one mRNA sequence encoding at least one antigenic peptide or protein derived from the glycoprotein (GP) or fragments, variants or derivatives thereof and the second dose containing at least one mRNA sequence encoding at least one antigenic peptide or protein derived from a different antigen of Ebolavirus or Marburgvirus, preferably from the matrix protein 40 (VP40) and/or the nucleoprotein (NP) (or fragments, variants or derivatives thereof). By that embodiment, both doses are administered in a staggered way, i.e. subsequently, shortly one after the other, e.g. within less than 10 minutes, preferably less than 2 minutes, and at the same site of the body to achieve the same immunological effect as for administration of one single composition containing both, e.g. the mRNA sequence encoding the glycoprotein (GP) and the mRNA sequence encoding the matrix protein 40 (VP40) and/or the nucleoprotein (NP).

According to a specific embodiment, the inventive mRNA sequence, or the inventive pharmaceutical composition or vaccine may be administered to the patient as a single dose or as at least one single dose, respectively. In certain embodiments, the inventive mRNA sequence or the inventive pharmaceutical composition or vaccine may be administered to a patient as a single dose followed by a second dose later and optionally even a third, fourth (or more) dose subsequent thereto etc. In accordance with this embodiment, booster inoculations with the inventive mRNA sequence or the inventive pharmaceutical composition or vaccine may be administered to a patient at specific time intervals, preferably as defined below, following the second (or third, fourth, etc.) inoculation.

Preferably, at least one dose of the inventive mRNA sequence, pharmaceutical composition or vaccine is administered, preferably from 1 to 10 doses, more preferably from 2 to 7 doses, even more preferably from 2 to 5 doses and most preferably from 3 to 5 doses. In a particularly preferred embodiment, 3 doses are administered. In another embodiment 2 doses are administered. In this context, it is particularly preferred that several doses comprise the same mRNA sequence encoding the same antigenic peptide or protein of Ebolavirus or Marburgvirus, e.g. glycoprotein (GP). In that embodiment, the doses are given in a specific time period, e.g. 20-30 or 20-60 days. The interval between the administration of two or more doses is preferably from 5 to 120 days, more preferably from 7 to 15 days or 15 to 30 days. In a preferred embodiment, the interval between the administration of two or more doses is at least 7 days, more preferably 28 days. For example, for post-exposure prophylaxis at least 5 doses of the inventive mRNA sequence or inventive pharmaceutical composition or vaccine can be administered within 20-30 days. As an example, for prophylactic treatment without exposure to the Ebolavirus or Marburgvirus at least 3 doses of the inventive mRNA sequence or the inventive pharmaceutical composition or vaccine can be administered in 20-60 days.

In a preferred embodiment, a single dose of the inventive mRNA sequence, composition or vaccine comprises a specific amount of the mRNA sequence according to the invention. Preferably, the inventive mRNA sequence is provided in an amount of at least 40 μg per dose, preferably in an amount of from 40 to 700 μg per dose, more preferably in an amount of from 80 to 400 μg per dose. More specifically, in the case of intradermal injection, which is preferably carried out by using a conventional needle, the amount of the inventive mRNA sequence comprised in a single dose is typically at least 200 μg, preferably from 200 μg to 1.000 μg, more preferably from 300 μg to 850 μg, even more preferably from 300 μg to 700 μg. In the case of intradermal injection, which is preferably carried out via jet injection (e.g. using a Tropis device; PharmaJet Inc, Boulder CO, US), the amount of the inventive mRNA sequence comprised in a single dose is typically at least 80 μg, preferably from 80 μg to 700 μg, more preferably from 80 μg to 400 μg. Moreover, in the case of intramuscular injection, which is preferably carried out by using a conventional needle or via jet injection, the amount of the inventive mRNA sequence comprised in a single dose is typically at least 80 μg, preferably from 80 μg to 1.000 μg, more preferably from 80 μg to 850 μg, even more preferably from 80 μg to 700 μg.

More specifically, the following specific embodiments are particularly preferred:
the inventive mRNA sequence, or the inventive pharmaceutical composition or vaccine is administered to the patient, preferably intradermally, in three doses (40 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intradermally, in three doses (80 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intradermally, in three doses (160 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intradermally, in three doses (320 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intradermally by jet injection, in three doses (40 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intradermally by jet injection, in three doses (80 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intradermally by jet injection, in three doses (160 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intradermally by jet injection, in three doses (320 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intramuscularly, in three doses (40 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intramuscularly in three doses (80 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intramuscularly, in three doses (160 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intramuscularly, in three doses (320 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intramuscularly, in three doses (640 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intramuscularly by jet injection, in three doses (40 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intramuscularly by jet injection, in three doses (80 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intramuscularly by jet injection, in three doses (160 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intramuscularly by jet injection, in three doses (320 μg/dose), preferably within 20-60 days, e.g. on day 0, 7 and 28 or on day 0, 28 and 56 of the treatment.
the inventive mRNA sequence, or the inventive pharmaceutical composition or vaccine is administered to the patient, preferably intramuscularly by jet injection, in three doses (640 μg/dose), preferably within 20-60 days, e.g. on day 0, 7 and 28 or on day 0, 28 and 56 of the treatment.

In certain embodiments, such booster inoculations with the inventive mRNA sequence or inventive pharmaceutical composition or vaccine as disclosed above (second, third etc. vaccination) may utilize an additional compound or component as defined for the inventive mRNA sequence or inventive pharmaceutical composition or vaccine as defined herein.

According to a further aspect, the present invention also provides a method for expression of an encoded antigenic peptide or protein derived from glycoprotein (GP) and/or matrix protein 40 (VP40) and/or nucleoprotein (NP) of a virus of the genus Ebolavirus or Marburgvirus comprising the steps, e.g. a) providing the inventive mRNA sequence as defined herein or the inventive composition comprising a plurality of inventive mRNA sequences as defined herein, b) applying or administering the inventive mRNA sequence as defined herein or the inventive composition comprising a plurality of inventive mRNA sequences as defined herein to an expression system, e.g. to a cell-free expression system, a cell (e.g. an expression host cell or a somatic cell), a tissue or an organism. The method may be applied for laboratory, for research, for diagnostic, for commercial production of peptides or proteins and/or for therapeutic purposes. In this context, typically after preparing the inventive mRNA sequence as defined herein or of the inventive composition comprising a plurality of inventive mRNA sequences as defined herein, it is typically applied or administered to a cell-free expression system, a cell (e.g. an expression host cell or a somatic cell), a tissue or an organism, e.g. in naked or complexed form or as a pharmaceutical composition or vaccine as described herein, preferably via transfection or by using any of the administration modes as described herein. The method may be carried out in vitro, in vivo or ex vivo. The method may furthermore be carried out in the context of the treatment of a specific disease, particularly in the treatment of infectious diseases, preferably Ebolavirus or Marburgvirus infections as defined herein.

In this context, in vitro is defined herein as transfection or transduction of the inventive mRNA as defined herein or of the inventive composition comprising a plurality of inventive mRNA sequences as defined herein into cells in culture outside of an organism; in vivo is defined herein as transfection or transduction of the inventive mRNA or of the inventive composition comprising a plurality of inventive mRNA sequences into cells by application of the inventive mRNA or of the inventive composition to the whole organism or individual and ex vivo is defined herein as transfection or transduction of the inventive mRNA or of the inventive composition comprising a plurality of inventive mRNA sequences into cells outside of an organism or individual and subsequent application of the transfected cells to the organism or individual.

Likewise, according to another aspect, the present invention also provides the use of the inventive mRNA sequence as defined herein or of the inventive composition comprising a plurality of inventive mRNA sequences as defined herein, preferably for diagnostic or therapeutic purposes, for expression of an encoded antigenic peptide or protein, e.g. by applying or administering the inventive mRNA sequence as defined herein or of the inventive composition comprising a plurality of inventive mRNA sequences as defined herein, e.g. to a cell-free expression system, a cell (e.g. an expression host cell or a somatic cell), a tissue or an organism. The use may be applied for laboratory, for research, for diagnostic for commercial production of peptides or proteins and/or for therapeutic purposes. In this context, typically after preparing the inventive mRNA sequence as defined herein or of the inventive composition comprising a plurality of inventive mRNA sequences as defined herein, it is typically applied or administered to a cell-free expression system, a cell (e.g. an expression host cell or a somatic cell), a tissue or an organism, preferably in naked form or complexed form, or as a pharmaceutical composition or vaccine as described herein, preferably via transfection or by using any of the administration modes as described herein. The use may be carried out in vitro, in vivo or ex vivo. The use may furthermore be carried out in the context of the treatment of a specific disease, particularly in the treatment of Ebolavirus or Marburgvirus infections.

In a further aspect the invention provides a method of treatment or prophylaxis of Ebolavirus or Marburgvirus infections comprising the steps:
a) providing the inventive mRNA sequence, the composition comprising a plurality of inventive mRNA sequences, the pharmaceutical composition or the kit or kit of parts comprising the inventive mRNA sequence as defined above;
b) applying or administering the mRNA sequence, the composition, the pharmaceutical composition or the kit or kit of parts to a tissue or an organism;
c) optionally administering Ebolavirus or Marburgvirus disease immune globuline.

Taken together the invention provides in a certain aspect an mRNA sequence comprising a coding region encoding at least one antigenic peptide or protein of Ebolavirus or Marburgvirus virus. The inventive mRNA sequence is for use in a method of prophylactic and/or therapeutic treatment of infections caused by Ebolaviruses or Marburgviruses. Accordingly, the invention relates to an mRNA sequence as defined herein for use in a method of prophylactic and/or therapeutic treatment of Ebolavirus or Marburgvirus infections.

In the present invention, if not otherwise indicated, different features of alternatives and embodiments may be combined with each other, where suitable. Furthermore, the term “comprising” shall not be narrowly construed as being limited to “consisting of” only, if not specifically mentioned. Rather, in the context of the present invention, “consisting of” is an embodiment specifically contemplated by the inventors to fall under the scope of “comprising”, wherever “comprising” is used herein.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### FIGURES

The figures shown in the following are merely illustrative and shall describe the present invention in a further way. These figures shall not be construed to limit the present invention thereto.

FIG. 1: shows the DNA sequence 32L-EBOV GP, Mayinga, Zaire 1976 (GC)-albumin7-A64-N5-C30-histoneSL-N5 according to SEQ ID NO. 37, comprising a G/C optimized coding region coding for Ebola virus glycoprotein (GP), the 32L TOP 5′-UTR element according to SEQ ID NO: 32, the 3′-UTR element albumin7 according to SEQ ID NO. 33, a poly (A) sequence consisting of 64 adenosines, a poly(C) sequence consisting of 30 cytosines and a histone stem-loop sequence according to SEQ ID NO. 35, corresponding to the inventive mRNA sequence coding for the glycoprotein (GP) of Ebola virus, Mayinga Zaire 1976.

FIG. 2: shows the DNA sequence 32L-EBOV GP, Sierra Leone 2014 (GC)-albumin7-A64-N5-C30-histoneSL-N5 according to SEQ ID NO. 38, comprising a G/C optimized coding region coding for Ebola virus glycoprotein (GP), the 32L TOP 5′-UTR element according to SEQ ID NO: 32, the 3′-UTR element albumin7 according to SEQ ID NO. 33, a poly (A) sequence consisting of 64 adenosines, a poly(C) sequence consisting of 30 cytosines and a histone stem-loop sequence according to SEQ ID NO. 35, corresponding to the inventive mRNA sequence coding for the glycoprotein (GP) of Ebola virus, Sierra Leone 2014.

FIG. 3: shows the DNA sequence 32L-MARV GP, Angola 2005 (GC)-albumin7-A64-N5-C30-histoneSL-N5 according to SEQ ID NO. 39, comprising a G/C optimized coding region coding for Marburg virus glycoprotein (GP), the 32L TOP 5′-UTR element according to SEQ ID NO: 32, the 3′-UTR element albumin7 according to SEQ ID NO. 33, a poly (A) sequence consisting of 64 adenosines, a poly(C) sequence consisting of 30 cytosines and a histone stem-loop sequence according to SEQ ID NO. 35, corresponding to the inventive mRNA sequence coding for the glycoprotein (GP) of Marburg virus, Angola 2005.

FIG. 4: shows the DNA sequence 32L-EBOV VP40, Mayinga, Zaire 1976 (GC)-albumin7-A64-N5-C30-histoneSL-N5 according to SEQ ID NO. 40, comprising a G/C optimized coding region coding for Ebola virus VP40 protein, the 32L TOP 5′-UTR element according to SEQ ID NO: 32, the 3′-UTR element albumin7 according to SEQ ID NO. 33, a poly (A) sequence consisting of 64 adenosines, a poly(C) sequence consisting of 30 cytosines and a histone stem-loop sequence according to SEQ ID NO. 35, corresponding to the inventive mRNA sequence coding for the VP40 protein of Ebola virus, Mayinga, Zaire 1976.

FIG. 5: shows the DNA sequence 32L-EBOV VP40, Sierra Leone 2014 (GC)-albumin7-A64-N5-C30-histoneSL-N5 according to SEQ ID NO. 41, comprising a G/C optimized coding region coding for Ebola virus VP40 protein, the 32L TOP 5′-UTR element according to SEQ ID NO: 32, the 3′-UTR element albumin7 according to SEQ ID NO. 33, a poly (A) sequence consisting of 64 adenosines, a poly(C) sequence consisting of 30 cytosines and a histone stem-loop sequence according to SEQ ID NO. 35, corresponding to the inventive mRNA sequence coding for the VP40 protein of Ebola virus, Sierra Leone 2014.

FIG. 6: shows the DNA sequence 32L-MARV VP40, Angola 2005 (GC)-albumin7-A64-N5-C30-histoneSL-N5 according to SEQ ID NO. 42, comprising a G/C optimized coding region coding for Marburg virus VP40 protein, the 32L TOP 5′-UTR element according to SEQ ID NO: 32, the 3′-UTR element albumin7 according to SEQ ID NO. 33, a poly (A) sequence consisting of 64 adenosines, a poly(C) sequence consisting of 30 cytosines and a histone stem-loop sequence according to SEQ ID NO. 35, corresponding to the inventive mRNA sequence coding for the VP40 protein of Marburg virus, Angola 2005.

FIG. 7: shows the DNA sequence 32L-EBOV NP, Zaire 1976 (GC)-albumin7-A64-N5-C30-histoneSL-N5 according to SEQ ID NO. 43, comprising a G/C optimized coding region coding for Ebola virus nucleoprotein (NP), the 32L TOP 5′-UTR element according to SEQ ID NO: 32, the 3′-UTR element albumin7 according to SEQ ID NO. 33, a poly (A) sequence consisting of 64 adenosines, a poly(C) sequence consisting of 30 cytosines and a histone stem-loop sequence according to SEQ ID NO. 35, corresponding to the inventive mRNA sequence coding for the nucleoprotein (NP) of Ebola virus, Zaire 1976.

FIG. 8: shows the DNA sequence 32L-EBOV NP, Sierra Leone 2014 (GC)-albumin7-A64-N5-C30-histoneSL-N5 according to SEQ ID NO. 44, comprising a G/C optimized coding region coding for Ebola virus nucleoprotein (NP), the 32L TOP 5′-UTR element according to SEQ ID NO: 32, the 3′-UTR element albumin7 according to SEQ ID NO. 33, a poly (A) sequence consisting of 64 adenosines, a poly(C) sequence consisting of 30 cytosines and a histone stem-loop sequence according to SEQ ID NO. 35, corresponding to the inventive mRNA sequence coding for the nucleoprotein (NP) of Ebola virus, Sierra Leone 2014.

FIG. 9: shows that that upon transfection of Hela cells with the mRNAs encoding Ebola virus glycoprotein (EBOV GP), expression of the glycoprotein can be detected on the surface of the transfected cells. The transfected cells were stained with an EBOV GP-specific antibody followed by a FITC labeled secondary antibody and analyzed by FACS. R3874 construct (SEQ ID NO: 45) encodes EBOV GP Mayinga-Zaire 1976, R3876 construct (SEQ ID NO: 46) encodes EBOV GP wt-SLE-2014 ManoRiver-NM042 Sierra Leone 2014. R2630 (SEQ ID NO: 233) encoding the influenza HA protein, served as a negative control. Geometric mean fluorescence (GMFI) of the surface expression is shown.

FIG. 10: shows that upon transfection of Hela cells with the mRNAs encoding Ebola virus glycoprotein (EBOV GP), expression of the full length GP1.2 protein can be detected by western blot in cell lysates and cell culture supernatants. R3874 construct (SEQ ID NO: 45) encodes EBOV GP Mayinga-Zaire 1976, R3876 construct (SEQ ID NO: 46) encodes EBOV GP wt-SLE-2014 ManoRiver-NM042 Sierra Leone 2014. The transfected cells were stained with mouse anti-EBOV GPdTM monoclonal antibody followed by secondary goat anti-mouse IgG (H+L) IRDye 800CW. Moreover, the presence of β-actin was analyzed as control for cellular contamination of the supernatants in combination with secondary goat anti-rabbit IgG(H+L) IRDye 680RD. Cells transfected with water for injection (WFI) were used as a negative control. Recombinant EBOV GPdTM protein was used an additional control. MM, molecular weight marker.

FIG. 11: shows humoral immune responses induced upon immunization of mice with mRNA vaccines encoding EBOV GP. Mice were immunized i.d. with 80 μg of the respective formulated RNA vaccine encoding EBOV GP Mayinga-Zaire 1976 (R3874; SEQ ID NO: 45) and EBOV GP wt-SLE-2014 ManoRiver-NM042 Sierra Leone 2014 (R3876; SEQ ID NO: 46) administered in a prime/boost/boost regimen on day 0, 21 and 42. RiLa buffer treated mice were used as control. EBOV GP-specific specific IgG1 (A) and IgG2a (B) titers were determined on day 56 by ELISA using recombinant EBOV GPdTM for coating. The horizontal bar indicates the median.

### EXAMPLES

The examples shown in the following are merely illustrative and shall describe the present invention in a further way. These examples shall not be construed to limit the present invention thereto.

### Example 1: Preparation of the Ebola and/or Marburg Virus mRNA Vaccine

1. Preparation of DNA and mRNA Constructs

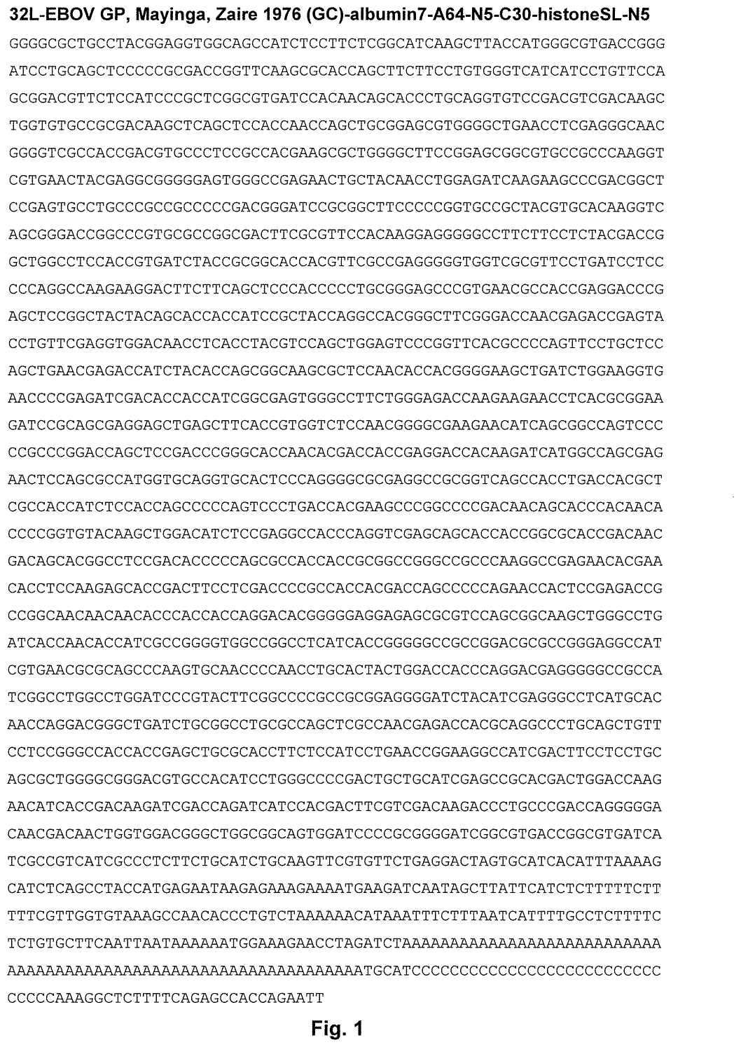

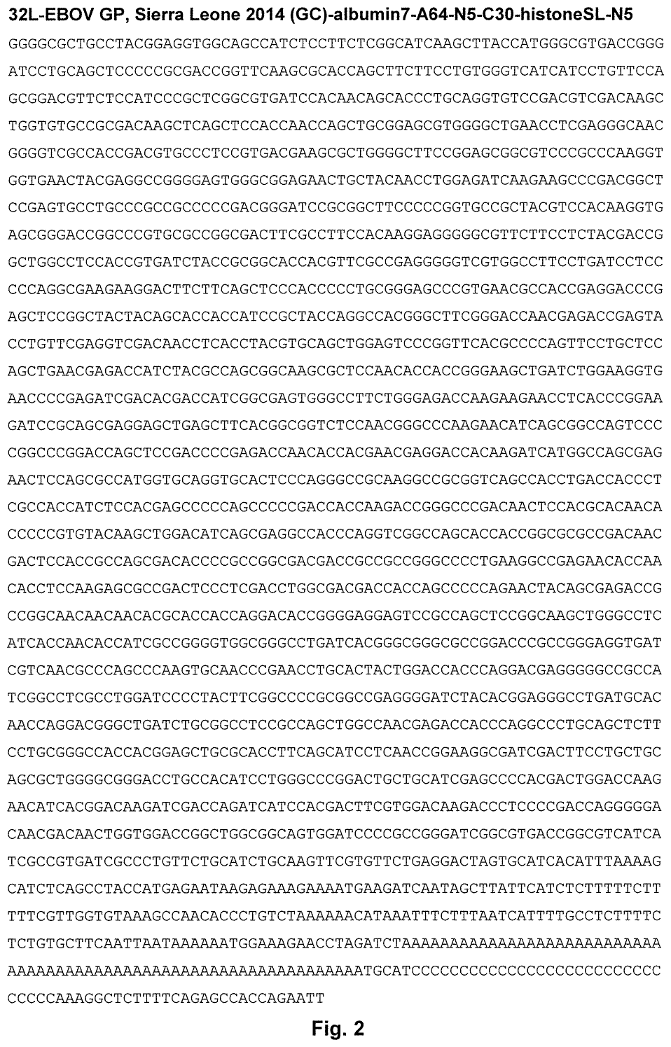

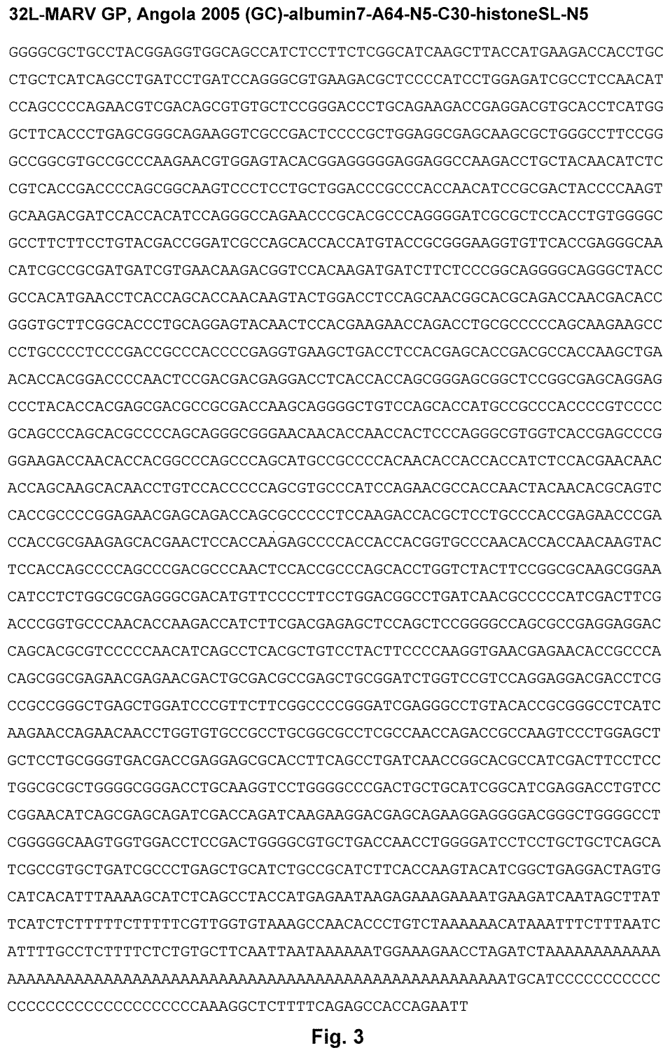

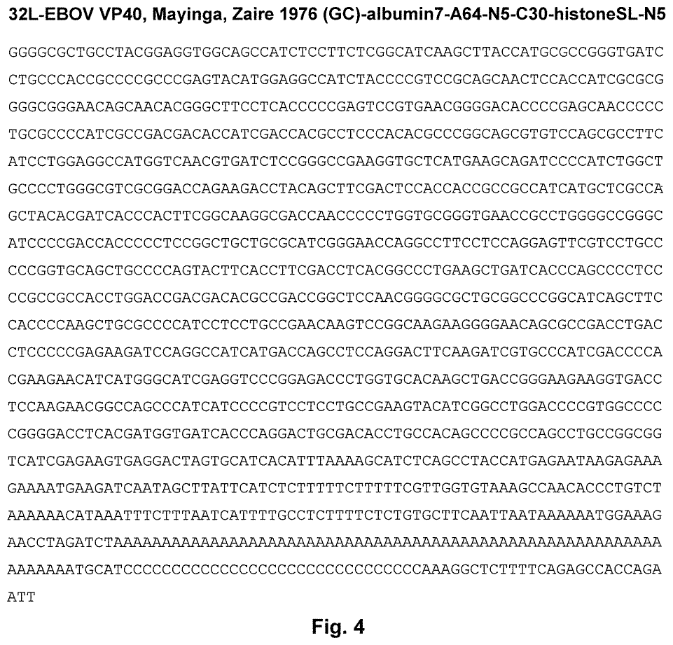

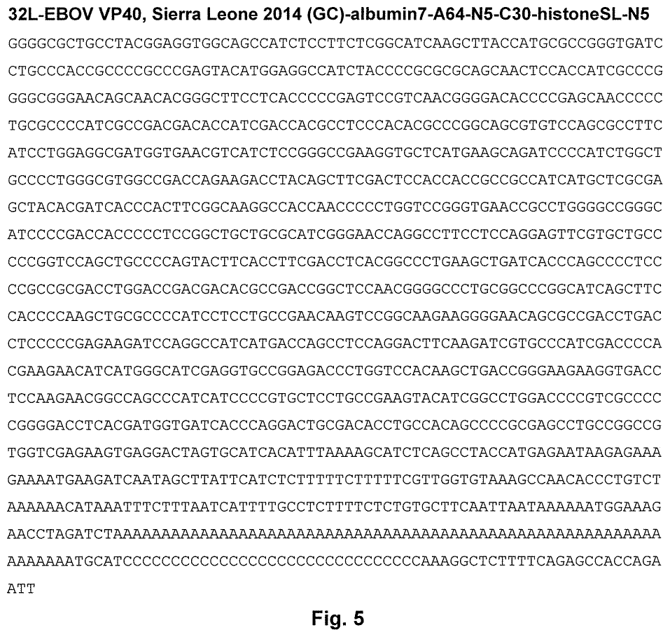

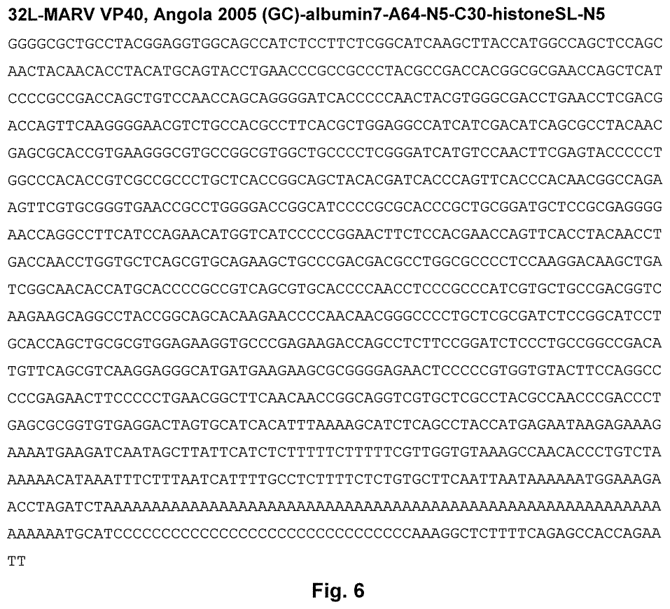

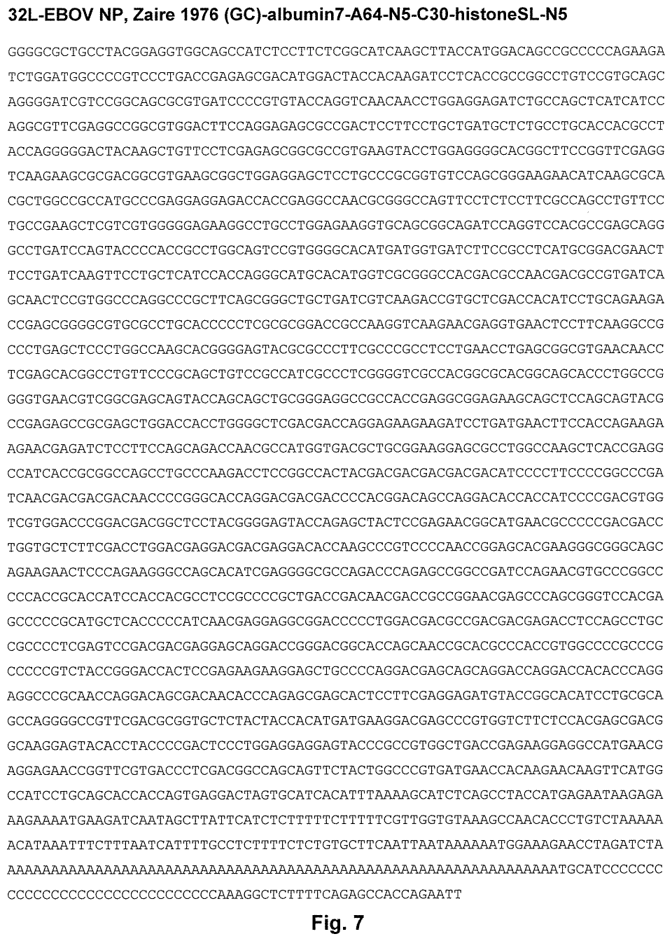

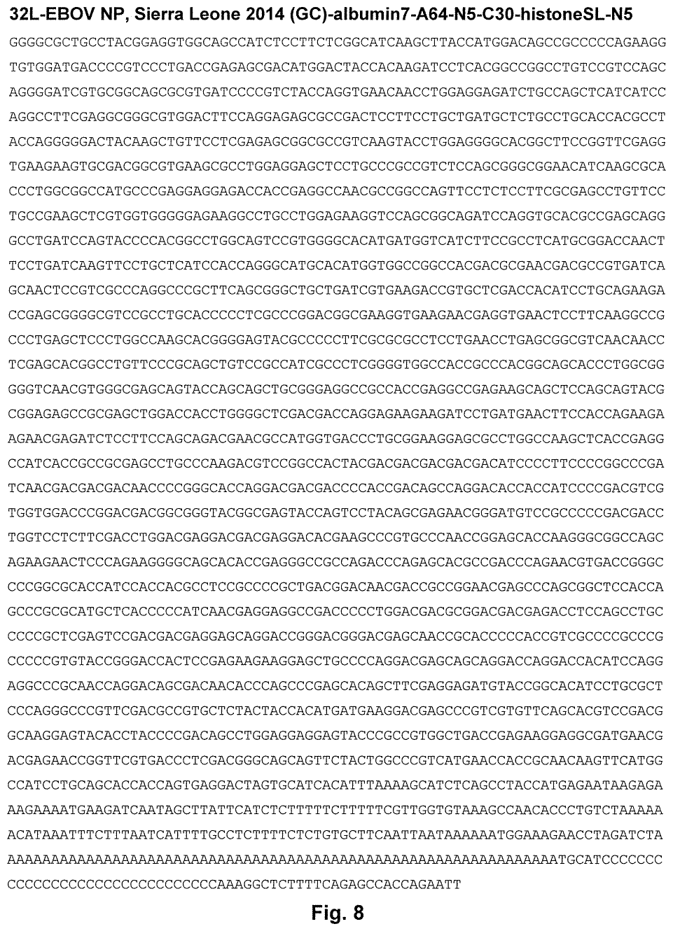

For the present examples DNA sequences, encoding glycoprotein (GP), matrix protein 40 (VP40) and/or nucleoprotein (NP) of different strains of Ebola virus and/or Marburg virus were prepared and used for subsequent in vitro transcription. The corresponding DNA sequences are shown in FIGS. 1 to 8 according to SEQ. ID No. 37 to 44

### 2. In Vitro Transcription

The respective DNA plasmids prepared according to paragraph 1 were transcribed in vitro using T7 polymerase in the presence of a CAP analogue (m⁷GpppG). Subsequently the mRNA was purified using PureMessenger® (CureVac, Tübingen, Germany; WO 2008/077592A1).

The mRNA sequences comprise in 5′- to 3′-direction:
a.) a 5′-CAP structure, consisting of m7GpppN;
b.) a 5′-UTR element comprising the corresponding RNA sequence of the nucleic acid sequence according to SEQ ID NO. 32;
c.) a G/C-maximized coding region encoding the full-length protein
d.) a 3′-UTR element comprising the corresponding RNA sequence of a nucleic acid sequence according to SEQ ID NO. 33;
e.) a poly(A) sequence, comprising 64 adenosines;
f.) a poly(C) sequence, comprising 30 cytosines; and
g.) a histone-stem-loop structure, comprising the RNA sequence according to SEQ ID No 35.

### 3. Reagents

Complexation Reagent: protamine

### 4. Preparation of the Vaccine

The mRNA sequences are complexed with protamine by addition of protamine to the mRNA in the ratio (1:2) (w/w) (adjuvant component). After incubation for 10 min, the same amount of free mRNA used as antigen-providing mRNA is added.

### Example 2: In Vitro Characterization of mRNA Encoding GP, VP40 and NP

Hela cells are seeded in a 6-well plate at a density of 400000 cells/well in cell culture medium (RPMI, 10% FCS, 1% L-Glutamine, 1% Pen/Strep) 24 h prior to transfection. HeLa cells are transfected with 1 or 2 μg of GP, VP40 or NP encoding mRNA with a buffer transfected sample as negative control using Lipofectamine 2000 (Invitrogen) and stained 24 hours post transfection with antigen specific antibodies and fluorescence labelled secondary antibody and analysed by flow cytometry (FACS). The flow cytometry data are evaluated quantitatively by FlowJo software.

For analysis of GP protein size and VLP formation induced by VP40, transfected cells are lysed and analysed for protein expression via western blotting using antigen specific antibodies.

### Example 3: Induction of a Humoral Immune Response by Ebola- and Marburgvirus Vaccines

### Immunization

On day zero, BALB/c mice are injected with mRNA vaccines comprising mRNA coding for GP, VP40 or NP alone or in combination. Mice are boosted twice on d21 and d42, respectively. Animals are analysed for antigen specific CD4+ and CD8+ T-cell responses 7 day post last boost as well as for antibody responses up to d70 post last boost.

TABLE 1
Animal groups

| | | Strain | | Vaccination |
|---|---|---|---|---|
| Group | Vaccine | sex | Number of mice | schedule |
| 1 | EBOV GP 1976 | BALB/c female | 8/8 | d0, d21, d42 |
| 2 | EBOV GP 2014 | BALB/c female | 8/8 | d0, d21, d42 |
| 3 | MARV GP | BALB/c female | 8/8 | d0, d21, d42 |
| 4 | EBOV GP 1976 + VP40 | BALB/c female | 8/8 | d0, d21, d42 |
| 5 | EBOV GP 2014 + VP40 | BALB/c female | 8/8 | d0, d21, d42 |
| 6 | MARV GP + VP40 | BALB/c female | 8/8 | d0, d21, d42 |
| 7 | EBOV GP 1976 + VP40 + NP | BALB/c female | 8/8 | d0, d21, d42 |
| 8 | EBOV GP 2014 + VP40 + NP | BALB/c female | 8/8 | d0, d21, d42 |
| 9 | EBOV GP 1976/VP40/NP | BALB/c female | 8/8 | d0, d21, d42 |
| | polycistronic IRES | | | |
| 9 | EBOV GP 1976/VP40/NP | BALB/c female | 8/8 | d0, d21, d42 |
| | polycistronic F2A | | | |
| 10 | EBOV GP 2014/VP40/NP | BALB/c female | 8/8 | d0, d21, d42 |
| | polycistronic IRES | | | |
| 11 | EBOV GP 2014/VP40/NP | BALB/c female | 8/8 | d0, d21, d42 |
| | polycistronic F2A | | | |
| 12 | MARV GP/VP40 bicistronic | BALB/c female | 8/8 | d0, d21, d42 |
| | IRES | | | |
| 13 | RiLa | BALB/c female | 8/8 | d0, d21, d42 |

### Example 4: Expression of Ebola Virus Glycoprotein—FACS Analysis

### 1. Cell Transfection

24 h prior to transfection Hela cells were seeded in a 6-well plate at a density of 4×10⁵ cells/well in cell culture medium (RPMI, 10% FCS, 1% L-Glutamine, 1% Pen/Strep). Hela cells were transfected with 1 and 2 μg formulated mRNA using Lipofectamine 2000 (Invitrogen). As a negative control, an irrelevant RNA (R2630; SEQ ID NO: 233) encoding the influenza HA protein or water for injection (WFI) was used.

### 2. FACS

Flow cytometric staining was performed 20-24 hours day post transfection using a mouse anti-EBOV GPdTM monoclonal antibody (Clone 4F3) followed by a secondary anti-mouse FITC-conjugated antibody (Sigma Aldrich). The samples were subsequently analyzed by flow cytometry (FACS) on BD FACS Canto II using the FACS Diva software. Quantitative analysis of the fluorescent FITC signal was performed using FlowJo software (Tree Star, Inc.).

### Results:

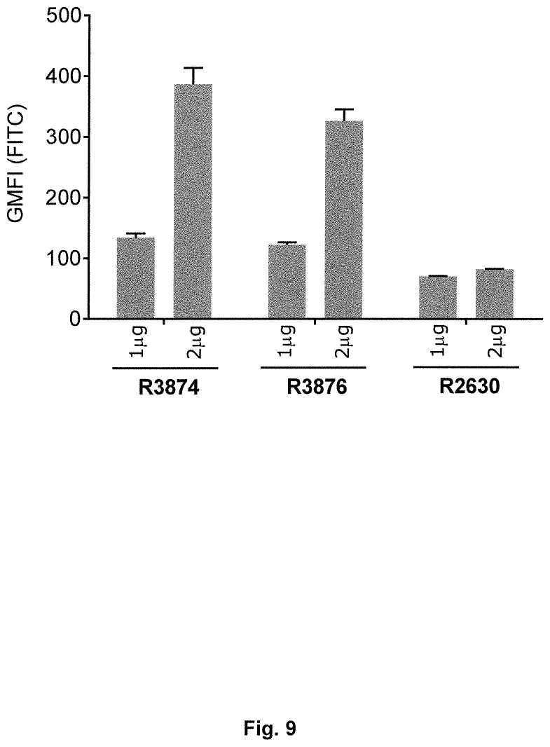

For both of the tested mRNA constructs encoding the glycoprotein from Ebola virus stain Mayinga-Zaire 1976 (R3874; SEQ ID NO: 45) or wt-SLE-2014 ManoRiver-NM042 Sierra Leone 2014 (R3876; SEQ ID NO: 46) EBOV GP expression was detectable by FACS analysis on the surface of the transfected Hela cells (see FIG. 9).

### Example 5: Expression of Ebola Virus Glycoprotein—Western Blot Analysis

### 1. Cell Transfection

24 h prior to transfection Hela cells were seeded in a 6-well plate at a density of 4×10⁵ cells/well in cell culture medium (RPMI, 10% FCS, 1% L-Glutamine, 1% Pen/Strep). Hela cells were transfected with 1 and 2 μg formulated mRNA using Lipofectamine 2000 (Invitrogen). As a negative control, an irrelevant RNA (R2630; SEQ ID NO: 233) encoding the influenza HA protein or water for injection (WFI) was used.

### 2. Western Blot

20-24 hours post transfection, cell culture supernatants were harvested, Hela cells were washed with PBS, detached by trypsin-free/EDTA buffer and cell pellets were lysed. Cell lysates and supernatants were subjected to SDS-PAGE under denaturating and reducing conditions. Western blot detection was carried out using mouse anti-EBOV GPdTM monoclonal antibody (Clone 4F3, IBT Bioservices) followed by goat anti-mouse IgG (H+L) IRDye 800CW (LI-COR Biosciences). The presence of β-actin was analyzed as control for cellular contamination of the supernatants using rabbit anti-β-actin antibody (cell Signalling Technology) in combination with secondary goat anti-rabbit IgG (H+L) IRDye 680RD (LI-COR Biosciences). Detection was carried out using an Odyssey CLx image system (LI-COR Biosciences).

### Results:

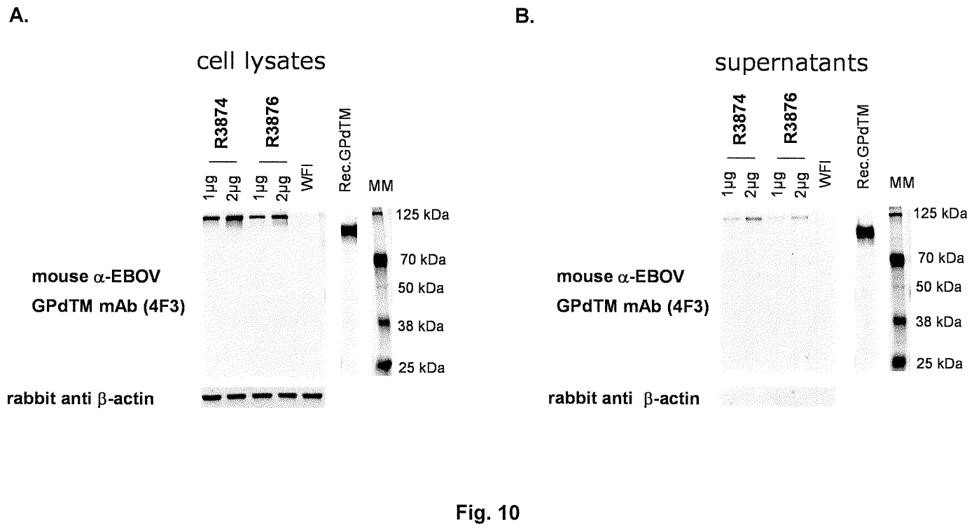

For both of the tested mRNA constructs encoding the glycoprotein from Ebola virus stain Mayinga-Zaire 1976 (R3874; SEQ ID NO: 45) or wt-SLE-2014 ManoRiver-NM042 Sierra Leone 2014 (R3876; SEQ ID NO: 46) expression of the full length GP1.2 protein was detectable in cell lysates (see FIG. 10A). In the cell culture supernatants only one band indicating trace amounts of full length GP1.2 protein was detected (FIG. 10B). Bands at lower molecular weight indicating smaller secreted forms, i.e. sGP and ssGP were not observed in the cell culture supernatants of Hela cells transfected with mRNA constructs encoding EBOV GP.

### Example 6: Humoral Immune Responses Induced Upon i.d. Immunization of Mice with mRNA Vaccines Encoding EBOV GP

### 1. Immunization

Female BALB/c mice (n=8/group) were injected via the intradermal route (i.d.) on day 0, 21 and 42 with 80 μg formulated mRNA vaccines encoding EBOV GP proteins. As a negative control, one group of mice was vaccinated with buffer (ringer lactate). Blood samples were collected at several time points post vaccination for determination of antibody titers.

### 2. Determination of Anti-EBOV GP Antibodies by ELISA:

EBOV GP-specific IgG1 and IgG2a antibody responses were analyzed by ELISA. The ELISA was established using recombinant EBOV GPdTM (IBT Bioservices) for coating. Coated plates were incubated using respective serum dilutions, and binding of specific antibodies to EBOV GP antigen was detected using biotinylated isotype specific anti-mouse antibodies in combination with streptavidin-HRP with amplex substrate.

### Results:

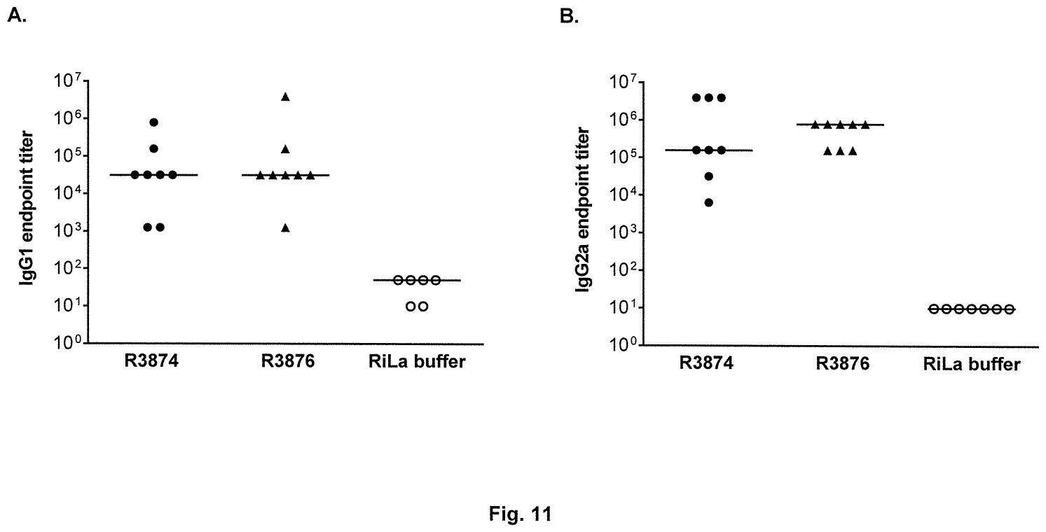

Assessment of the humoral immune response after intradermal immunizations revealed that 80 μg of the respective EBOV GP mRNA vaccines (R3874 (SEQ ID NO: 45) and R3876 (SEQ ID NO: 46)) induced comparable levels of EBOV GP-specific IgG1 and IgG2a antibody titers (see FIG. 11).

1. A mRNA comprising a coding region, encoding at least one antigenic peptide or protein derived from the glycoprotein (GP), the matrix protein 40 (VP40), or the nucleoprotein (NP) of a virus of the genus Ebolavirus or Marburgvirus or a fragment.

2. A pharmaceutical composition comprising the mRNA sequence of claim 1.

3. The mRNA of claim 1, wherein the coding region encodes the full-length protein of glycoprotein (GP), the matrix protein 40 (VP40), or the nucleoprotein (NP) of a virus of the genus Ebolavirus or Marburgvirus.

4. The mRNA claim 1, wherein the coding region encodes the full-length protein of glycoprotein (GP) of a virus of the genus Ebolavirus and wherein the coding region includes an editing site of seven consecutive adenosine residues and wherein one further adenosine residue is added to the editing site.

5. (canceled)

6. The mRNA of claim 1, wherein the antigenic peptide or protein is derived from the species Ebola ebolavirus (EBOV), Bundibugyo ebolavirus (BDBV), Sudan ebolavirus (SUDV), Taï Forest ebolavirus (TAFV), or Marburg marburgvirus (MARV).

7. The mRNA of claim 1, comprising additionally
a) a 5′-CAP structure,
b) a poly(A) sequence,
c) and optionally a poly (C) sequence.

8. The mRNA according to claim 7, wherein the poly(A) sequence comprises a sequence of about 25 to about 400 adenosine nucleotides.

9. The mRNA of claim 1, comprising additionally at least one histone stem-loop.

10. The mRNA of claim 1, comprising additionally a 3′-UTR element.

11. The mRNA according to claim 10, wherein the at least one 3′-UTR element comprises or consists of a nucleic acid sequence which is derived from a 3′-UTR of a gene providing a stable mRNA.

12. The mRNA according to claim 11, wherein the 3′-UTR element comprises a nucleic acid sequence derived from a 3′-UTR of a gene selected from the group consisting of an albumin gene, an α-globin gene, a β-globin gene, a tyrosine hydroxylase gene, a lipoxygenase gene, and a collagen alpha gene.

13. The mRNA of claim 10, wherein the 3′-UTR element is derived from a nucleic acid sequence according to SEQ ID NO. 33 or SEQ ID NO. 34.

14. The mRNA of claim 1, wherein the mRNA sequence comprises, in 5′- to 3′-direction:
a.) a 5′-CAP structure;
b.) a coding region encoding at least one antigenic peptide or protein of a virus of the genus Ebolavirus or Marburgvirus, wherein the peptide or protein is derived from the glycoprotein (GP), the matrix protein 40 (VP40), or the nucleoprotein (NP) of a virus of the genus Ebolavirus or Marburgvirus;
c.) a 3′-UTR element comprising or consisting of a nucleic acid sequence which is derived from an alpha globin gene, comprising the corresponding RNA sequence of the nucleic acid sequence according to SEQ ID NO. 34;
d.) optionally, a poly(A) sequence, preferably comprising 64 adenosines;
e.) optionally, a poly(C) sequence, preferably comprising 30 cytosines; and
f.) optionally, a histone-stem-loop, preferably comprising the corresponding RNA sequence to the nucleic acid sequence according to SEQ ID NO. 35.

15. The mRNA of claim 11, comprising additionally a 5′-UTR element which comprises a nucleic acid sequence which is derived from the 5′-UTR of a TOP gene lacking the 5′TOP motif.

16. (canceled)

17. The mRNA according to claim 15, wherein the 5′-UTR element comprises a nucleic acid sequence which is derived from a 5′-UTR of a TOP gene encoding a ribosomal Large protein (RPL), the nucleic acid sequence according to SEQ ID NO. 32.

18. (canceled)

19. The mRNA of claim 1, wherein the mRNA sequence is any of SEQ ID NOs: 37 to 44.

20. The mRNA of claim 1, wherein the mRNA sequence is associated with or complexed with a cationic or polycationic compound or a polymeric carrier.

21. The mRNA according to claim 20, wherein the mRNA sequence is associated or complexed with a cationic protein or peptide.

22. (canceled)

23. A pharmaceutical composition comprising an mRNA sequence of claim 1, and a pharmaceutically acceptable carrier.

24-31. (canceled)

32. A method of treatment or prophylaxis of Ebolavirus infections or Marburgvirus infections comprising the steps:
a) providing the mRNA sequence of claim 1; and
b) administering the mRNA sequence to a tissue or an organism.

33-34. (canceled)


SEQUENCE LISTING (233 sequences)

SEQ ID NO: 1 - protein, 676 aa, synthetic construct
  REGION 1..676: note = EBOV GP, Mayinga, Zaire 1976
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 2 - protein, 676 aa, synthetic construct
  REGION 1..676: note = EBOV GP, Sierra Leone 2014
MGVTGILQLPRDRFKRTSFFLWVIILFQRTFSIPLGVIHNSTLQVSDVDKLVCRDKLSSTNQLRSVGLNLEGNGVATDVPSVTKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYASGKRSNTTGKLIWKVNPEIDTTIGEWAFWETKKNLTRKIRSEELSFTAVSNGPKNISGQSPARTSSDPETNTTNEDHKIMASENSSAMVQVHSQGRKAAVSHLTTLATISTSPQPPTTKTGPDNSTHNTPVYKLDISEATQVGQHHRRADNDSTASDTPPATTAAGPLKAENTNTSKSADSLDLATTTSPQNYSETAGNNNTHHQDTGEESASSGKLGLITNTIAGVAGLITGGRRTRREVIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 3 - protein, 681 aa, synthetic construct
  REGION 1..681: note = MARV GP, Angola 2005
MKTTCLLISLILIQGVKTLPILEIASNIQPQNVDSVCSGTLQKTEDVHLMGFTLSGQKVADSPLEASKRWAFRAGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGTLQEYNSTKNQTCAPSKKPLPLPTAHPEVKLTSTSTDATKLNTTDPNSDDEDLTTSGSGSGEQEPYTTSDAATKQGLSSTMPPTPSPQPSTPQQGGNNTNHSQGVVTEPGKTNTTAQPSMPPHNTTTISTNNTSKHNLSTPSVPIQNATNYNTQSTAPENEQTSAPSKTTLLPTENPTTAKSTNSTKSPTTTVPNTTNKYSTSPSPTPNSTAQHLVYFRRKRNILWREGDMFPFLDGLINAPIDFDPVPNTKTIFDESSSSGASAEEDQHASPNISLTLSYFPKVNENTAHSGENENDCDAELRIWSVQEDDLAAGLSWIPFFGPGIEGLYTAGLIKNQNNLVCRLRRLANQTAKSLELLLRVTTEERTFSLINRHAIDFLLARWGGTCKVLGPDCCIGIEDLSRNISEQIDQIKKDEQKEGTGWGLGGKWWTSDWGVLTNLGILLLLSIAVLIALSCICRIFTKYIG

SEQ ID NO: 4 - protein, 676 aa, synthetic construct
  REGION 1..676: note = BDBV GP, Uganda 2007
MVTSGILQLPRERFRKTSFFVWVIILFHKVFPIPLGVVHNNTLQVSDIDKLVCRDKLSSTSQLKSVGLNLEGNGVATDVPTATKRWGFRAGVPPKVVNYEAGEWAENCYNLDIKKADGSECLPEAPEGVRGFPRCRYVHKVSGTGPCPEGYAFHKEGAFFLYDRLASTIIYRSTTFSEGVVAFLILPETKKDFFQSPPLHEPANMTTDPSSYYHTVTLNYVADNFGTNMTNFLFQVDHLTYVQLEPRFTPQFLVQLNETIYTNGRRSNTTGTLIWKVNPTVDTGVGEWAFWENKKNFTKTLSSEELSVIFVPRAQDPGSNQKTKVTPTSFANNQTSKNHEDLVPEDPASVVQVRDLQRENTVPTPPPDTVPTTLIPDTMEEQTTSHYEPPNISRNHQERNNTAHPETLANNPPDNTTPSTPPQDGERTSSHTTPSPRPVPTSTIHPTTRETHIPTTMTTSHDTDSNRPNPIDISESTEPGPLTNTTRGAANLLTGSRRTRREITLRTQAKCNPNLHYWTTQDEGAAIGLAWIPYFGPAAEGIYTEGIMHNQNGLICGLRQLANETTQALQLFLRATTELRTFSILNRKAIDFLLQRWGGTCHILGPDCCIEPHDWTKNITDKIDQIIHDFIDKPLPDQTDNDNWWTGWRQWVPAGIGITGVIIAVIALLCICKFLL

SEQ ID NO: 5 - protein, 676 aa, synthetic construct
  REGION 1..676: note = SUDV GP, Gulu, Uganda 2007
MGGLSLLQLPRDKFRKSSFFVWVIILFQKAFSMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKIDNNTFVRLDRPHTPQFLFQLNDTIHLHQQLSNTTGRLIWTLDANINADIGEWAFWENKKNLSEQLRGEELSFEALSLNETEDDDAASSRITKGRISDRATRKYSDLVPKNSPGMVPLHIPEGETTLPSQNSTEGRRVGVNTQETITETAATIIGTNGNHMQISTIGIRPSSSQIPSSSPTTAPSPEAQTPTTHTSGPSVMATEEPTTPPGSSPGPTTEAPTLTTPENITTAVKTVLPQESTSNGLITSTVTGILGSLGLRKRSRRQTNTKATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLC

SEQ ID NO: 6 - protein, 676 aa, synthetic construct
  REGION 1..676: note = TAFV GP, Cote dIvoire 1994
MGASGILQLPRERFRKTSFFVWVIILFHKVFSIPLGVVHNNTLQVSDIDKFVCRDKLSSTSQLKSVGLNLEGNGVATDVPTATKRWGFRAGVPPKVVNCEAGEWAENCYNLAIKKVDGSECLPEAPEGVRDFPRCRYVHKVSGTGPCPGGLAFHKEGAFFLYDRLASTIIYRGTTFAEGVIAFLILPKARKDFFQSPPLHEPANMTTDPSSYYHTTTINYVVDNFGTNTTEFLFQVDHLTYVQLEARFTPQFLVLLNETIYSDNRRSNTTGKLIWKINPTVDTSMGEWAFWENKKNFTKTLSSEELSFVPVPETQNQVLDTTATVSPPISAHNHAAEDHKELVSEDSTPVVQMQNIKGKDTMPTTVTGVPTTTPSPFPINARNTDHTKSFIGLEGPQEDHSTTQPAKTTSQPTNSTESTTLNPTSEPSSRGTGPSSPTVPNTTESHAELGKTTPTTLPEQHTAASAIPRAVHPDELSGPGFLTNTIRGVTNLLTGSRRKRRDVTPNTQPKCNPNLHYWTALDEGAAIGLAWIPYFGPAAEGIYTEGIMENQNGLICGLRQLANETTQALQLFLRATTELRTFSILNRKAIDFLLQRWGGTCHILGPDCCIEPQDWTKNITDKIDQIIHDFVDNNLPNQNDGSNWWTGWKQWVPAGIGITGVIIAIIALLCICKFML

SEQ ID NO: 7 - protein, 326 aa, synthetic construct
  REGION 1..326: note = EBOV VP40, Mayinga, Zaire 1976
MRRVILPTAPPEYMEAIYPVRSNSTIARGGNSNTGFLTPESVNGDTPSNPLRPIADDTIDHASHTPGSVSSAFILEAMVNVISGPKVLMKQIPIWLPLGVADQKTYSFDSTTAAIMLASYTITHFGKATNPLVRVNRLGPGIPDHPLRLLRIGNQAFLQEFVLPPVQLPQYFTFDLTALKLITQPLPAATWTDDTPTGSNGALRPGISFHPKLRPILLPNKSGKKGNSADLTSPEKIQAIMTSLQDFKIVPIDPTKNIMGIEVPETLVHKLTGKKVTSKNGQPIIPVLLPKYIGLDPVAPGDLTMVITQDCDTCHSPASLPAVIEK

SEQ ID NO: 8 - protein, 326 aa, synthetic construct
  REGION 1..326: note = EBOV VP40, Sierra Leone 2014
MRRVILPTAPPEYMEAIYPARSNSTIARGGNSNTGFLTPESVNGDTPSNPLRPIADDTIDHASHTPGSVSSAFILEAMVNVISGPKVLMKQIPIWLPLGVADQKTYSFDSTTAAIMLASYTITHFGKATNPLVRVNRLGPGIPDHPLRLLRIGNQAFLQEFVLPPVQLPQYFTFDLTALKLITQPLPAATWTDDTPTGSNGALRPGISFHPKLRPILLPNKSGKKGNSADLTSPEKIQAIMTSLQDFKIVPIDPTKNIMGIEVPETLVHKLTGKKVTSKNGQPIIPVLLPKYIGLDPVAPGDLTMVITQDCDTCHSPASLPAVVEK

SEQ ID NO: 9 - protein, 303 aa, synthetic construct
  REGION 1..303: note = MARV VP40, Angola 2005
MASSSNYNTYMQYLNPPPYADHGANQLIPADQLSNQQGITPNYVGDLNLDDQFKGNVCHAFTLEAIIDISAYNERTVKGVPAWLPLGIMSNFEYPLAHTVAALLTGSYTITQFTHNGQKFVRVNRLGTGIPAHPLRMLREGNQAFIQNMVIPRNFSTNQFTYNLTNLVLSVQKLPDDAWRPSKDKLIGNTMHPAVSVHPNLPPIVLPTVKKQAYRQHKNPNNGPLLAISGILHQLRVEKVPEKTSLFRISLPADMFSVKEGMMKKRGENSPVVYFQAPENFPLNGFNNRQVVLAYANPTLSAV

SEQ ID NO: 10 - protein, 326 aa, synthetic construct
  REGION 1..326: note = BDBV VP40, Uganda 2007
MRRAILPTAPPEYIEAVYPMRTVSTSINSTASGPNFPAPDVMMSDTPSNSLRPIADDNIDHPSHTPTSVSSAFILEAMVNVISGPKVLMKQIPIWLPLGVADQKTYSFDSTTAAIMLASYTITHFGKTSNPLVRINRLGPGIPDHPLRLLRIGNQAFLQEFVLPPVQLPQYFTFDLTALKLITQPLPAATWTDDTPTGPTGILRPGISFHPKLRPILLPGKTGKRGSSSDLTSPDKIQAIMNFLQDLKLVPIDPAKNIMGIEVPELLVHRLTGKKITTKNGQPIIPILLPKYIGMDPISQGDLTMVITQDCDTCHSPASLPPVSEK

SEQ ID NO: 11 - protein, 326 aa, synthetic construct
  REGION 1..326: note = SUDV VP40, Gulu, Uganda 2000
MRRVTVPTAPPAYADIGYPMSMLPIKSSRAVSGIQQKQEVLPGMDTPSNSMRPVADDNIDHTSHTPNGVASAFILEATVNVISGPKVLMKQIPIWLPLGIADQKTYSFDSTTAAIMLASYTITHFGKANNPLVRVNRLGQGIPDHPLRLLRMGNQAFLQEFVLPPVQLPQYFTFDLTALKLVTQPLPAATWTDETPSNLSGALRPGLSFHPKLRPVLLPGKTGKKGHVSDLTAPDKIQTIVNLMQDFKIVPIDPAKSIIGIEVPELLVHKLTGKKMSQKNGQPIIPVLLPKYIGLDPISPGDLTMVITPDYDDCHSPASCSYLSEK

SEQ ID NO: 12 - protein, 326 aa, synthetic construct
  REGION 1..326: note = TAFV VP40, Cote dIvoire 1994
MRRIILPTAPPEYMEAVYPMRTMNSGADNTASGPNYTTTGVMTNDTPSNSLRPVADDNIDHPSHTPNSVASAFILEAMVNVISGPKVLMKQIPIWLPLGVSDQKTYSFDSTTAAIMLASYTITHFGKTSNPLVRINRLGPGIPDHPLRLLRIGNQAFLQEFVLPPVQLPQYFTFDLTALKLITQPLPAATWTDETPAVSTGTLRPGISFHPKLRPILLPGRAGKKGSNSDLTSPDKIQAIMNFLQDLKIVPIDPTKNIMGIEVPELLVHRLTGKKTTTKNGQPIIPILLPKYIGLDPLSQGDLTMVITQDCDSCHSPASLPPVNEK

SEQ ID NO: 13 - protein, 739 aa, synthetic construct
  REGION 1..739: note = EBOV NP, Mayinga, Zaire 1976
MDSRPQKIWMAPSLTESDMDYHKILTAGLSVQQGIVRQRVIPVYQVNNLEEICQLIIQAFEAGVDFQESADSFLLMLCLHHAYQGDYKLFLESGAVKYLEGHGFRFEVKKRDGVKRLEELLPAVSSGKNIKRTLAAMPEEETTEANAGQFLSFASLFLPKLVVGEKACLEKVQRQIQVHAEQGLIQYPTAWQSVGHMMVIFRLMRTNFLIKFLLIHQGMHMVAGHDANDAVISNSVAQARFSGLLIVKTVLDHILQKTERGVRLHPLARTAKVKNEVNSFKAALSSLAKHGEYAPFARLLNLSGVNNLEHGLFPQLSAIALGVATAHGSTLAGVNVGEQYQQLREAATEAEKQLQQYAESRELDHLGLDDQEKKILMNFHQKKNEISFQQTNAMVTLRKERLAKLTEAITAASLPKTSGHYDDDDDIPFPGPINDDDNPGHQDDDPTDSQDTTIPDVVVDPDDGSYGEYQSYSENGMNAPDDLVLFDLDEDDEDTKPVPNRSTKGGQQKNSQKGQHIEGRQTQSRPIQNVPGPHRTIHHASAPLTDNDRRNEPSGSTSPRMLTPINEEADPLDDADDETSSLPPLESDDEEQDRDGTSNRTPTVAPPAPVYRDHSEKKELPQDEQQDQDHTQEARNQDSDNTQSEHSFEEMYRHILRSQGPFDAVLYYHMMKDEPVVFSTSDGKEYTYPDSLEEEYPPWLTEKEAMNEENRFVTLDGQQFYWPVMNHKNKFMAILQHHQ

SEQ ID NO: 14 - protein, 739 aa, synthetic construct
  REGION 1..739: note = EBOV NP, Sierra Leone 2014
MDSRPQKVWMTPSLTESDMDYHKILTAGLSVQQGIVRQRVIPVYQVNNLEEICQLIIQAFEAGVDFQESADSFLLMLCLHHAYQGDYKLFLESGAVKYLEGHGFRFEVKKCDGVKRLEELLPAVSSGRNIKRTLAAMPEEETTEANAGQFLSFASLFLPKLVVGEKACLEKVQRQIQVHAEQGLIQYPTAWQSVGHMMVIFRLMRTNFLIKFLLIHQGMHMVAGHDANDAVISNSVAQARFSGLLIVKTVLDHILQKTERGVRLHPLARTAKVKNEVNSFKAALSSLAKHGEYAPFARLLNLSGVNNLEHGLFPQLSAIALGVATAHGSTLAGVNVGEQYQQLREAATEAEKQLQQYAESRELDHLGLDDQEKKILMNFHQKKNEISFQQTNAMVTLRKERLAKLTEAITAASLPKTSGHYDDDDDIPFPGPINDDDNPGHQDDDPTDSQDTTIPDVVVDPDDGGYGEYQSYSENGMSAPDDLVLFDLDEDDEDTKPVPNRSTKGGQQKNSQKGQHTEGRQTQSTPTQNVTGPRRTIHHASAPLTDNDRRNEPSGSTSPRMLTPINEEADPLDDADDETSSLPPLESDDEEQDRDGTSNRTPTVAPPAPVYRDHSEKKELPQDEQQDQDHIQEARNQDSDNTQPEHSFEEMYRHILRSQGPFDAVLYYHMMKDEPVVFSTSDGKEYTYPDSLEEEYPPWLTEKEAMNDENRFVTLDGQQFYWPVMNHRNKFMAILQHHQ

SEQ ID NO: 15 - protein, 695 aa, synthetic construct
  REGION 1..695: note = MARV NP, Angola 2005
MDLHSLLELGTKPTAPHVRNKKVILFDTNHQVSICNQIIDAINSGIDLGDLLEGGLLTLCVEHYYNSDKDKFNTSPIAKYLRDAGYEFDVIKNADATRFLDVIPNEPHYSPLILALKTLESTESQRGRIGLFLSFCSLFLPKLVVGDRASIEKALRQVTVHQEQGIVTYPNHWLTTGHMKVIFGILRSSFILKFVLIHQGVNLVTGHDAYDSIISNSVGQTRFSGLLIVKTVLEFILQKTDSGVTLHPLVRTSKVKNEVASFKQALSNLARHGEYAPFARVLNLSGINNLEHGLYPQLSAIALGVATAHGSTLAGVNVGEQYQQLREAAHDAEVKLQRRHEHQEIQAIAEDDEERKILEQFHLQKTEITHSQTLAVLSQKREKLARLAAEIENNIVEDQGFKQSQNRVSQSFLNDPTPVEVTVQARPINRPTALPPPVDSKIEHESTEDSSSSSSFVDLNDPFALLNEDEDTLDDSVMIPSTTSREFQGIPEPPRQSQDIDNSQGKQEDESTNLIKKPFLRYQELPPVQEDDESEYTTDSQESIDQPGSDNEQGVDLPPPPLYAQEKRQDPIQHPAVSSQDPFGSIGDVNGDILEPIRSPSSPSAPQEDTRAREAYELSPDFTNYEDNQQNWPQRVVTKKGRTFLYPNDLLQTNPPESLITALVEEYQNPVSAKELQADWPDMSFDERRHVAMNL

SEQ ID NO: 16 - protein, 739 aa, synthetic construct
  REGION 1..739: note = BDBV NP, Uganda 2007
MDPRPIRTWMMHNTSEVEADYHKILTAGLSVQQGIVRQRIIPVYQISNLEEVCQLIIQAFEAGVDFQDSADSFLLMLCLHHAYQGDYKQFLESNAVKYLEGHGFRFEMKKKEGVKRLEELLPAASSGKNIKRTLAAMPEEETTEANAGQFLSFASLFLPKLVVGEKACLEKVQRQIQVHAEQGLIQYPTSWQSVGHMMVIFRLMRTNFLIKFLLIHQGMHMVAGHDANDAVIANSVAQARFSGLLIVKTVLDHILQKTEHGVRLHPLARTAKVKNEVSSFKAALASLAQHGEYAPFARLLNLSGVNNLEHGLFPQLSAIALGVATAHGSTLAGVNVGEQYQQLREAATEAEKQLQKYAESRELDHLGLDDQEKKILKDFHQKKNEISFQQTTAMVTLRKERLAKLTEAITSTSILKTGRRYDDDNDIPFPGPINDNENSGQNDDDPTDSQDTTIPDVIIDPNDGGYNNYSDYANDAASAPDDLVLFDLEDEDDADNPAQNTPEKNDRPATTKLRNGQDQDGNQGETASPRVAPNQYRDKPMPQVQDRSENHDQTLQTQSRVLTPISEEADPSDHNDGDNESIPPLESDDEGSTDTTAAETKPATAPPAPVYRSISVDDSVPSENIPAQSNQTNNEDNVRNNAQSEQSIAEMYQHILKTQGPFDAILYYHMMKEEPIIFSTSDGKEYTYPDSLEDEYPPWLSEKEAMNEDNRFITMDGQQFYWPVMNHRNKFMAILQHHR

SEQ ID NO: 17 - protein, 738 aa, synthetic construct
  REGION 1..738: note = SUDV NP, Gulu, Uganda 2000
MDKRVRGSWALGGQSEVDLDYHKILTAGLSVQQGIVRQRVIPVYVVSDLEGICQHIIQAFEAGVDFQDNADSFLLLLCLHHAYQGDHRLFLKSDAVQYLEGHGFRFEVREKENVHRLDELLPNVTGGKNLRRTLAAMPEEETTEANAGQFLSFASLFLPKLVVGEKACLEKVQRQIQVHAEQGLIQYPTSWQSVGHMMVIFRLMRTNFLIKFLLIHQGMHMVAGHDANDTVISNSVAQARFSGLLIVKTVLDHILQKTDLGVRLHPLARTAKVKNEVSSFKAALGSLAKHGEYAPFARLLNLSGVNNLEHGLYPQLSAIALGVATAHGSTLAGVNVGEQYQQLREAATEAEKQLQQYAETRELDNLGLDEQEKKILMSFHQKKNEISFQQTNAMVTLRKERLAKLTEAITTASKIKVGDRYPDDNDIPFPGPIYDETHPNPSDDNPDDSRDTTIPGGVVDPYDDESNNYPDYEDSAEGTTGDLDLFNLDDDDDDSQPGPPDRGQSKERAARTHGLQDPTLDGAKKVPELTPGSHQPGNLHITKPGSNTNQPQGNMSSTLQSMTPIQEESEPDDQKDDDDESLTSLDSEGDEDVESVSGENNPTVAPPAPVYKDTGVDTNQQNGPSNAVDGQGSESEALPINPEKGSALEETYYHLLKTQGPFEAINYYHLMSDEPIAFSTESGKEYIFPDSLEEAYPPWLSEKEALEKENRYLVIDGQQFLWPVMSLQDKFLAVLQHD

SEQ ID NO: 18 - protein, 739 aa, synthetic construct
  REGION 1..739: note = TAFV NP, Cote dIvoire 1994
MESRAHKAWMTHTASGFETDYHKILTAGLSVQQGIVRQRVIQVHQVTNLEEICQLIIQAFEAGVDFQESADSFLLMLCLHHAYQGDYKQFLESNAVKYLEGHGFRFEVRKKEGVKRLEELLPAASSGKSIRRTLAAMPEEETTEANAGQFLSFASLFLPKLVVGEKACLEKVQRQIQVHSEQGLIQYPTAWQSVGHMMVIFRLMRTNFLIKFLLIHQGMHMVAGHDANDAVIANSVAQARFSGLLIVKTVLDHILQKTEHGVRLHPLARTAKVKNEVNSFKAALSSLAQHGEYAPFARLLNLSGVNNLEHGLFPQLSAIALGVATAHGSTLAGVNVGEQYQQLREAATEAEKQLQKYAESRELDHLGLDDQEKKILKDFHQKKNEISFQQTTAMVTLRKERLAKLTEAITSTSLLKTGKQYDDDNDIPFPGPINDNENSEQQDDDPTDSQDTTIPDIIVDPDDGRYNNYGDYPSETANAPEDLVLFDLEDGDEDDHRPSSSSENNNKHSLTGTDSNKTSNWNRNPTNMPKKDSTQNNDNPAQRAQEYARDNIQDTPTPHRALTPISEETGSNGHNEDDIDSIPPLESDEENNTETTITTTKNTTAPPAPVYRSNSEKEPLPQEKSQKQPNQVSGSENTDNKPHSEQSVEEMYRHILQTQGPFDAILYYYMMTEEPIVFSTSDGKEYVYPDSLEGEHPPWLSEKEALNEDNRFITMDDQQFYWPVMNHRNKFMAILQHHK

SEQ ID NO: 19 - DNA, 38 bp, synthetic construct
  misc_feature 1..38: note = Insertion nucleotide sequence (SEQ ID NO. 19)
  source 1..38: mol_type = other DNA
acctcactagaaaaattcgcagtgaagagttgtctttc

SEQ ID NO: 20 - DNA, 2031 bp, synthetic construct
  misc_feature 1..2031: note = EBOV GP, Mayinga, Zaire 1976, Modified wild type nucleotide sequence of the coding region
  source 1..2031: mol_type = other DNA
atgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttag

SEQ ID NO: 21 - DNA, 2031 bp, synthetic construct
  misc_feature 1..2031: note = EBOV GP, Sierra Leone 2014, Modified wild type nucleotide sequence of the coding region
  source 1..2031: mol_type = other DNA
atgggtgttacaggaatattgcagttacctcgtgatcgattcaagaggacatcattctttctttgggtaattatccttttccaaagaacattttccatcccgcttggagttatccacaatagtacattacaggttagtgatgtcgacaaactagtttgtcgtgacaaactgtcatccacaaatcaattgagatcagttggactgaatctcgaggggaatggagtggcaactgacgtgccatctgtgactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccatgtgccggagactttgccttccacaaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtcgagtggctattattctaccacaattagatatcaggctaccggttttggaactaatgagacagagtacttgttcgaggttgacaatttgacctacgtccaacttgaatcaagattcacaccacagtttctgctccagctgaatgagacaatatatgcaagtgggaagaggagcaacaccacgggaaaactaatttggaaggtcaaccccgaaattgatacaacaatcggggagtgggccttctgggaaactaaaaaaaacctcactagaaaaattcgcagtgaagagttgtctttcacagctgtatcaaacggacccaaaaacatcagtggtcagagtccggcgcgaacttcttccgacccagagaccaacacaacaaatgaagaccacaaaatcatggcttcagaaaattcctctgcaatggttcaagtgcacagtcaaggaaggaaagctgcagtgtcgcatctgacaacccttgccacaatctccacgagtcctcaacctcccacaaccaaaacaggtccggacaacagcacccataatacacccgtgtataaacttgacatctctgaggcaactcaagttggacaacatcaccgtagagcagacaacgacagcacagcctccgacactccccccgccacgaccgcagccggacccttaaaagcagagaacaccaacacgagtaagagcgctgactccctggacctcgccaccacgacaagcccccaaaactacagcgagactgctggcaacaacaacactcatcaccaagataccggagaagagagtgccagcagcgggaagctaggcttaattaccaatactattgctggagtagcaggactgatcacaggcgggagaaggactcgaagagaagtaattgtcaatgctcaacccaaatgcaaccccaatttacattactggactactcaggatgaaggtgctgcaatcggattggcctggataccatatttcgggccagcagccgaaggaatttacacagaggggctaatgcacaaccaagatggtttaatctgtgggttgaggcagctggccaacgaaacgactcaagctctccaactgttcctgagagccacaactgagctgcgaaccttttcaatcctcaaccgtaaggcaattgacttcctgctgcagcgatggggtggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggtgttataattgcagttatcgctttattctgtatatgcaaatttgtcttttag

SEQ ID NO: 22 - DNA, 2046 bp, synthetic construct
  misc_feature 1..2046: note = MARV GP, Angola 2005, Wild type nucleotide sequence of the coding region
  source 1..2046: mol_type = other DNA
atgaaaaccacatgtctccttatcagtcttatcttaatccaaggggtaaaaactctccctattttagagatagccagtaacattcaaccccaaaatgtggattcagtatgctccgggactctccagaagacagaagacgttcatctgatgggattcacactgagcgggcaaaaagttgctgattcccctttagaggcatccaaacgatgggccttcagggcaggtgtacctcccaagaatgttgagtatacagaaggggaggaagctaaaacatgttacaatataagtgtaacggatccctctggaaaatccttgctgttagatcctcctaccaacatccgtgactatcctaaatgcaaaactatccatcatattcaaggtcaaaaccctcatgcacaggggatcgctctccatttgtggggagcatttttcttgtatgatcgcatcgcctccacaacgatgtatcgaggcaaagtcttcactgaagggaacatagcagctatgattgtcaataagacagtgcacaaaatgattttctcgaggcaaggacaagggtaccgtcacatgaacctaacttctactaataaatattggacaagtagcaacggaacgcaaacgaatgacactggatgcttcggtactcttcaagaatataattctacaaagaaccaaacatgtgctccgtccaaaaaacctttaccactgcccacagcccatccggaggtcaagctcactagcacctcaactgatgccaccaaactcaataccacagacccaaacagtgatgatgaggacctcacaacatctggctcagggtctggagaacaggaaccttacacaacttctgacgcagccacgaagcaagggctttcatcaacaatgccgcccactccctcaccacaaccaagcacgccacagcaaggaggaaacaacacgaaccattcccaaggtgttgtgactgaacccggcaaaaccaacacaactgcacaaccgtccatgccccctcacaacactactacaatctctactaacaacacctccaagcacaacctcagcactccctctgtaccaatacaaaatgccactaattacaacacacagagcacggcccctgaaaatgagcaaaccagtgccccctcgaaaacaaccctgcttccaacagaaaatcctacaacagcaaagagcaccaatagtacaaaaagccccactacaacagtaccaaatacgacaaataagtattccaccagtccctcccccacccccaactcgactgcacaacatcttgtatatttcagaaggaaacgaaatattctctggagggaaggcgacatgttcccttttctggatgggttaataaatgctccgattgattttgatccggttccaaatacaaagacaatctttgatgaatcctctagttctggtgcttcagctgaggaagatcagcatgcctcccctaatatcagtttaactttatcttactttcctaaggtaaatgaaaacactgcccactctggagaaaatgaaaatgattgtgatgcagagttaagaatttggagtgttcaggaggacgacctggcagcaggactcagttggataccgttttttggccctggaatcgaaggactttatactgctggtttaattaaaaatcaaaataatttggtttgcaggttgaggcgtctagccaatcagactgccaaatccttggaactcttattaagagtcacaaccgaggaaagaacattttccttaatcaatagacatgccattgattttttactcgcaaggtggggaggaacatgcaaagtgcttggacctgattgttgcatcggaatagaagacttgtccagaaatatttcagaacaaattgatcaaatcaaaaaggacgaacaaaaagaggggactggttggggtctgggtggtaaatggtggacatcagactggggtgttcttactaacttgggcatcttgctactactgtccatagctgtcttaattgctctgtcctgtatttgtcgtatttttactaaatatattggataa

SEQ ID NO: 23 - DNA, 981 bp, synthetic construct
  misc_feature 1..981: note = EBOV VP40, Mayinga, Zaire 1976, Wild type nucleotide sequence of the coding region
  source 1..981: mol_type = other DNA
atgaggcgggttatattgcctactgctcctcctgaatatatggaggccatataccctgtcaggtcaaattcaacaattgctagaggtggcaacagcaatacaggcttcctgacaccggagtcagtcaatggggacactccatcgaatccactcaggccaattgccgatgacaccatcgaccatgccagccacacaccaggcagtgtgtcatcagcattcatccttgaagctatggtgaatgtcatatcgggccccaaagtgctaatgaagcaaattccaatttggcttcctctaggtgtcgctgatcaaaagacctacagctttgactcaactacggccgccatcatgcttgcttcatacactatcacccatttcggcaaggcaaccaatccacttgtcagagtcaatcggctgggtcctggaatcccggatcatcccctcaggctcctgcgaattggaaaccaggctttcctccaggagttcgttcttccgccagtccaactaccccagtatttcacctttgatttgacagcactcaaactgatcacccaaccactgcctgctgcaacatggaccgatgacactccaacaggatcaaatggagcgttgcgtccaggaatttcatttcatccaaaacttcgccccattcttttacccaacaaaagtgggaagaaggggaacagtgccgatctaacatctccggagaaaatccaagcaataatgacttcactccaggactttaagatcgttccaattgatccaaccaaaaatatcatgggaatcgaagtgccagaaactctggtccacaagctgaccggtaagaaggtgacttctaaaaatggacaaccaatcatccctgttcttttgccaaagtacattgggttggacccggtggctccaggagacctcaccatggtaatcacacaggattgtgacacgtgtcattctcctgcaagtcttccagctgtgattgagaagtaa

SEQ ID NO: 24 - DNA, 981 bp, synthetic construct
  misc_feature 1..981: note = EBOV VP40, Sierra Leone 2014, Wild type nucleotide sequence of the coding region
  source 1..981: mol_type = other DNA
atgaggcgggttatattgcctactgctcctcctgaatatatggaggccatataccctgccaggtcaaattcaacaattgctaggggtggcaacagcaatacaggcttcctgacaccggagtcagtcaatggagacactccatcgaatccactcaggccaattgctgatgacaccatcgaccatgccagccacacaccaggcagtgtgtcatcagcattcatcctcgaagctatggtgaatgtcatatcgggccccaaagtgctaatgaagcaaattccaatttggcttcctctaggtgtcgctgatcaaaagacctacagctttgactcaactacggccgccatcatgcttgcttcatatactatcacccatttcggcaaggcaaccaatccgcttgtcagagtcaatcggctgggtcctggaatcccggatcaccccctcaggctcctgcgaattggaaaccaggctttcctccaggagttcgttcttccaccagtccaactaccccagtatttcacctttgatttgacagcactcaaactgatcactcaaccactgcctgctgcaacatggaccgatgacactccaactggatcaaatggagcgttgcgtccaggaatttcatttcatccaaaacttcgccccattcttttacccaacaaaagtgggaagaaggggaacagtgccgatctaacatctccggagaaaatccaagcaataatgacttcactccaggactttaagatcgttccaattgatccaaccaaaaatatcatgggtatcgaagtgccagaaactctggtccacaagctgaccggtaagaaggtgacttccaaaaatggacaaccaatcatccctgttcttttgccaaagtacattgggttggacccggtggctccaggagacctcaccatggtaatcacacaggattgtgacacgtgtcattctcctgcaagtcttccagctgtggttgagaagtaa

SEQ ID NO: 25 - DNA, 912 bp, synthetic construct
  misc_feature 1..912: note = MARV VP40, Angola 2005, Wild type nucleotide sequence of the coding region
  source 1..912: mol_type = other DNA
atggccagttccagcaattacaatacatacatgcaataccttaacccccctccttatgctgaccacggtgcaaaccagttaatcccggcggatcagctatcaaatcagcagggtataactccaaattatgtgggtgatttaaacctagatgaccagttcaaagggaatgtctgccatgctttcactttagaggcaataattgacatatctgcgtataacgaacgaacagtcaaaggcgttccggcatggctgcctcttgggatcatgagcaatttcgaatatcctttagcccatacagtggctgcgttgctcacaggcagctatacaatcacccagtttactcataatgggcaaaaattcgtccgtgtcaatcgactcggtacaggaatcccggcacacccactcaggatgttgcgtgaaggaaatcaagcttttattcagaatatggtgatccccaggaatttttccaccaatcaattcacctacaatctcactaacttagtattgagtgtgcaaaaacttcctgatgatgcctggcgtccgtccaaggacaaattaattggaaacaccatgcatcctgcagtctccgttcacccgaatttaccgcctattgttctaccaacagtcaagaagcaggcttatcgccagcacaaaaatcccaacaatggtccactgctggccatatctggcatccttcatcaactgagagtcgaaaaagtcccagaaaagacaagcctgtttaggatttcgcttcctgccgacatgttctcagtaaaagagggtatgatgaagaaaagaggagaaaattccccggtagtttattttcaagcacctgagaacttccctttgaatggcttcaacaacagacaagttgtactagcgtatgcgaatccaacactcagcgccgtttaa

SEQ ID NO: 26 - DNA, 2220 bp, synthetic construct
  misc_feature 1..2220: note = EBOV NP, Zaire 1976, Wild type nucleotide sequence of the coding region
  source 1..2220: mol_type = other DNA
atggattctcgtcctcagaaaatctggatggcgccgagtctcactgaatctgacatggattaccacaagatcttgacagcaggtctgtccgttcaacaggggattgttcggcaaagagtcatcccagtgtatcaagtaaacaatcttgaagaaatttgccaacttatcatacaggcctttgaagcaggtgttgattttcaagagagtgcggacagtttccttctcatgctttgtcttcatcatgcgtaccagggagattacaaacttttcttggaaagtggcgcagtcaagtatttggaagggcacgggttccgttttgaagtcaagaagcgtgatggagtgaagcgccttgaggaattgctgccagcagtatctagtggaaaaaacattaagagaacacttgctgccatgccggaagaggagacaactgaagctaatgccggtcagtttctctcctttgcaagtctattccttccgaaattggtagtaggagaaaaggcttgccttgagaaggttcaaaggcaaattcaagtacatgcagagcaaggactgatacaatatccaacagcttggcaatcagtaggacacatgatggtgattttccgtttgatgcgaacaaattttctgatcaaatttctcctaatacaccaagggatgcacatggttgccgggcatgatgccaacgatgctgtgatttcaaattcagtggctcaagctcgtttttcaggcttattgattgtcaaaacagtacttgatcatatcctacaaaagacagaacgaggagttcgtctccatcctcttgcaaggaccgccaaggtaaaaaatgaggtgaactcctttaaggctgcactcagctccctggccaagcatggagagtatgctcctttcgcccgacttttgaacctttctggagtaaataatcttgagcatggtcttttccctcaactatcggcaattgcactcggagtcgccacagcacacgggagtaccctcgcaggagtaaatgttggagaacagtatcaacaactcagagaggctgccactgaggctgagaagcaactccaacaatatgcagagtctcgcgaacttgaccatcttggacttgatgatcaggaaaagaaaattcttatgaacttccatcagaaaaagaacgaaatcagcttccagcaaacaaacgctatggtaactctaagaaaagagcgcctggccaagctgacagaagctatcactgctgcgtcactgcccaaaacaagtggacattacgatgatgatgacgacattccctttccaggacccatcaatgatgacgacaatcctggccatcaagatgatgatccgactgactcacaggatacgaccattcccgatgtggtggttgatcccgatgatggaagctacggcgaataccagagttactcggaaaacggcatgaatgcaccagatgacttggtcctattcgatctagacgaggacgacgaggacactaagccagtgcctaatagatcgaccaagggtggacaacagaagaacagtcaaaagggccagcatatagagggcagacagacacaatccaggccaattcaaaatgtcccaggccctcacagaacaatccaccacgccagtgcgccactcacggacaatgacagaagaaatgaaccctccggctcaaccagccctcgcatgctgacaccaattaacgaagaggcagacccactggacgatgccgacgacgagacgtctagccttccgcccttggagtcagatgatgaagagcaggacagggacggaacttccaaccgcacacccactgtcgccccaccggctcccgtatacagagatcactctgaaaagaaagaactcccgcaagacgagcaacaagatcaggaccacactcaagaggccaggaaccaggacagtgacaacacccagtcagaacactcttttgaggagatgtatcgccacattctaagatcacaggggccatttgatgctgttttgtattatcatatgatgaaggatgagcctgtagttttcagtaccagtgatggcaaagagtacacgtatccagactcccttgaagaggaatatccaccatggctcactgaaaaagaggctatgaatgaagagaatagatttgttacattggatggtcaacaattttattggccggtgatgaatcacaagaataaattcatggcaatcctgcaacatcatcagtga

SEQ ID NO: 27 - DNA, 2220 bp, synthetic construct
  misc_feature 1..2220: note = EBOV NP, Sierra Leone 2014, Wild type nucleotide sequence of the coding region
  source 1..2220: mol_type = other DNA
atggattctcgtcctcagaaagtctggatgacgccgagtctcactgaatctgacatggattaccacaagatcttgacagcaggtctgtccgttcaacaggggattgttcggcaaagagtcatcccagtgtatcaagtaaacaatcttgaggaaatttgccaacttatcatacaggcctttgaagctggtgttgattttcaagagagtgcggacagtttccttctcatgctttgtcttcatcatgcgtaccaaggagattacaaacttttcttggaaagtggcgcagtcaagtatttggaagggcacgggttccgttttgaagtcaagaagtgtgatggagtgaagcgccttgaggaattgctgccagcagtatctagtgggagaaacattaagagaacacttgctgccatgccggaagaggagacgactgaagctaatgccggtcagttcctctcctttgcaagtctattccttccgaaattggtagtaggagaaaaggcttgccttgagaaggttcaaaggcaaattcaagtacatgcagagcaaggactgatacaatatccaacagcttggcaatcagtaggacacatgatggtgattttccgtttgatgcgaacaaattttttgatcaaatttcttctaatacaccaagggatgcacatggttgccggacatgatgccaacgatgctgtgatttcaaattcagtggctcaagctcgtttttcaggtctattgattgtcaaaacagtacttgatcatatcctacaaaagacagaacgaggagttcgtctccatcctcttgcaaggaccgccaaggtaaaaaatgaggtgaactccttcaaggctgcactcagctccctggccaagcatggagagtatgctcctttcgcccgacttttgaacctttctggagtaaataatcttgagcatggtcttttccctcaactgtcggcaattgcactcggagtcgccacagcccacgggagcaccctcgcaggagtaaatgttggagaacagtatcaacagctcagagaggcagccactgaggctgagaagcaactccaacaatatgcggagtctcgtgaacttgaccatcttggacttgatgatcaggaaaagaaaattcttatgaacttccatcagaaaaagaacgaaatcagcttccagcaaacaaacgcgatggtaactctaagaaaagagcgcctggccaagctgacagaagctatcactgctgcatcactgcccaaaacaagtggacattacgatgatgatgacgacattccctttccaggacccatcaatgatgacgacaatcctggccatcaagatgatgatccgactgactcacaggatacgaccattcccgatgtggtagttgaccccgatgatggaggctacggcgaataccaaagttactcggaaaacggcatgagtgcaccagatgacttggtcctattcgatctagacgaggacgacgaggacaccaagccagtgcctaacagatcgaccaagggtggacaacagaaaaacagtcaaaagggccagcatacagagggcagacagacacaatccacgccaactcaaaacgtcacaggccctcgcagaacaatccaccatgccagtgctccactcacggacaatgacagaagaaacgaaccctccggctcaaccagccctcgcatgctgaccccaatcaacgaagaggcagacccactggacgatgccgacgacgagacgtctagccttccgcccttagagtcagatgatgaagaacaggacagggacggaacttctaaccgcacacccactgtcgccccaccggctcccgtatacagagatcactccgaaaagaaagaactcccgcaagatgaacaacaagatcaggaccacattcaagaggccaggaaccaagacagtgacaacacccagccagaacattcttttgaggagatgtatcgccacattctaagatcacaggggccatttgatgccgttttgtattatcatatgatgaaggatgagcctgtagttttcagtaccagtgatggtaaagagtacacgtatccggactcccttgaagaggaatatccaccatggctcactgaaaaagaggccatgaatgatgagaatagatttgttacactggatggtcaacaattttattggccagtaatgaatcacaggaataaattcatggcaatcctgcaacatcatcagtga

SEQ ID NO: 28 - DNA, 671 bp, synthetic construct
  misc_feature 1..671: note = Nucleotide sequence of IRES of EMCV
  source 1..671: mol_type = other DNA
ttgaaagccgggggtgggagatccggattgccagtctgctcgatatcgcaggctgggtccgtgactacccactccccctttaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatagatctacc

SEQ ID NO: 29 - DNA, 461 bp, synthetic construct
  misc_feature 1..461: note = Nucleotide sequence of IRES of FMDV
  source 1..461: mol_type = other DNA
agcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttacaattaaagaccct

SEQ ID NO: 30 - DNA, 66 bp, synthetic construct
  misc_feature 1..66: note = Nucleotide sequence of F2A peptide, version 1, of FMDV
  source 1..66: mol_type = other DNA
gtgaagcagacactcaatttcgaccttctgaagttggctggagatgttgagtctaacccaggcccc

SEQ ID NO: 31 - DNA, 66 bp, synthetic construct
  misc_feature 1..66: note = Nucleotide sequence of F2A peptide, version 2, of FMDV
  source 1..66: mol_type = other DNA
gtcaaacagaccttgaacttcgacttgctcaaactggccggggatgtggagtccaatcctggacct

SEQ ID NO: 32 - DNA, 42 bp, synthetic construct
  misc_feature 1..42: note = Nucleotide sequence for 5-UTR element
  source 1..42: mol_type = other DNA
ggcgctgcctacggaggtggcagccatctccttctcggcatc

SEQ ID NO: 33 - DNA, 186 bp, synthetic construct
  misc_feature 1..186: note = Nucleotide sequence of 3-UTR element of human albumin gene
  source 1..186: mol_type = other DNA
catcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 34 - DNA, 44 bp, synthetic construct
  misc_feature 1..44: note = Nucleotide sequence of 3 UTR element of an a-globin gene
  source 1..44: mol_type = other DNA
gcccgatgggcctcccaacgggccctcctcccctccttgcaccg

SEQ ID NO: 35 - DNA, 24 bp, synthetic construct
  misc_feature 1..24: note = Histone stem-loop nucleotide sequence
  source 1..24: mol_type = other DNA
caaaggctcttttcagagccacca

SEQ ID NO: 36 - RNA, 24 nt, synthetic construct
  misc_feature 1..24: note = Histone stem-loop RNA sequence
  source 1..24: mol_type = other RNA
caaaggctcttttcagagccacca

SEQ ID NO: 37 - DNA, 2413 bp, synthetic construct
  misc_feature 1..2413: note = EBOV GP, Mayinga, Zaire 1976, optimized nucleotide sequence
  source 1..2413: mol_type = other DNA
ggggcgctgcctacggaggtggcagccatctccttctcggcatcaagcttaccatgggcgtgaccgggatcctgcagctcccccgcgaccggttcaagcgcaccagcttcttcctgtgggtcatcatcctgttccagcggacgttctccatcccgctcggcgtgatccacaacagcaccctgcaggtgtccgacgtcgacaagctggtgtgccgcgacaagctcagctccaccaaccagctgcggagcgtggggctgaacctcgagggcaacggggtcgccaccgacgtgccctccgccacgaagcgctggggcttccggagcggcgtgccgcccaaggtcgtgaactacgaggcgggggagtgggccgagaactgctacaacctggagatcaagaagcccgacggctccgagtgcctgcccgccgcccccgacgggatccgcggcttcccccggtgccgctacgtgcacaaggtcagcgggaccggcccgtgcgccggcgacttcgcgttccacaaggagggggccttcttcctctacgaccggctggcctccaccgtgatctaccgcggcaccacgttcgccgagggggtggtcgcgttcctgatcctcccccaggccaagaaggacttcttcagctcccaccccctgcgggagcccgtgaacgccaccgaggacccgagctccggctactacagcaccaccatccgctaccaggccacgggcttcgggaccaacgagaccgagtacctgttcgaggtggacaacctcacctacgtccagctggagtcccggttcacgccccagttcctgctccagctgaacgagaccatctacaccagcggcaagcgctccaacaccacggggaagctgatctggaaggtgaaccccgagatcgacaccaccatcggcgagtgggccttctgggagaccaagaagaacctcacgcggaagatccgcagcgaggagctgagcttcaccgtggtctccaacggggcgaagaacatcagcggccagtcccccgcccggaccagctccgacccgggcaccaacacgaccaccgaggaccacaagatcatggccagcgagaactccagcgccatggtgcaggtgcactcccaggggcgcgaggccgcggtcagccacctgaccacgctcgccaccatctccaccagcccccagtccctgaccacgaagcccggccccgacaacagcacccacaacaccccggtgtacaagctggacatctccgaggccacccaggtcgagcagcaccaccggcgcaccgacaacgacagcacggcctccgacacccccagcgccaccaccgcggccgggccgcccaaggccgagaacacgaacacctccaagagcaccgacttcctcgaccccgccaccacgaccagcccccagaaccactccgagaccgccggcaacaacaacacccaccaccaggacacgggggaggagagcgcgtccagcggcaagctgggcctgatcaccaacaccatcgccggggtggccggcctcatcaccgggggccgccggacgcgccgggaggccatcgtgaacgcgcagcccaagtgcaaccccaacctgcactactggaccacccaggacgagggggccgccatcggcctggcctggatcccgtacttcggccccgccgcggaggggatctacatcgagggcctcatgcacaaccaggacgggctgatctgcggcctgcgccagctcgccaacgagaccacgcaggccctgcagctgttcctccgggccaccaccgagctgcgcaccttctccatcctgaaccggaaggccatcgacttcctcctgcagcgctggggcgggacgtgccacatcctgggccccgactgctgcatcgagccgcacgactggaccaagaacatcaccgacaagatcgaccagatcatccacgacttcgtcgacaagaccctgcccgaccagggggacaacgacaactggtggacgggctggcggcagtggatccccgcggggatcggcgtgaccggcgtgatcatcgccgtcatcgccctcttctgcatctgcaagttcgtgttctgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 38 - DNA, 2413 bp, synthetic construct
  misc_feature 1..2413: note = EBOV GP, Sierra Leone 2014, optimized nucleotide sequence
  source 1..2413: mol_type = other DNA
ggggcgctgcctacggaggtggcagccatctccttctcggcatcaagcttaccatgggcgtgaccgggatcctgcagctcccccgcgaccggttcaagcgcaccagcttcttcctgtgggtcatcatcctgttccagcggacgttctccatcccgctcggcgtgatccacaacagcaccctgcaggtgtccgacgtcgacaagctggtgtgccgcgacaagctcagctccaccaaccagctgcggagcgtggggctgaacctcgagggcaacggggtcgccaccgacgtgccctccgtgacgaagcgctggggcttccggagcggcgtcccgcccaaggtggtgaactacgaggccggggagtgggcggagaactgctacaacctggagatcaagaagcccgacggctccgagtgcctgcccgccgcccccgacgggatccgcggcttcccccggtgccgctacgtccacaaggtgagcgggaccggcccgtgcgccggcgacttcgccttccacaaggagggggcgttcttcctctacgaccggctggcctccaccgtgatctaccgcggcaccacgttcgccgagggggtcgtggccttcctgatcctcccccaggcgaagaaggacttcttcagctcccaccccctgcgggagcccgtgaacgccaccgaggacccgagctccggctactacagcaccaccatccgctaccaggccacgggcttcgggaccaacgagaccgagtacctgttcgaggtcgacaacctcacctacgtgcagctggagtcccggttcacgccccagttcctgctccagctgaacgagaccatctacgccagcggcaagcgctccaacaccaccgggaagctgatctggaaggtgaaccccgagatcgacacgaccatcggcgagtgggccttctgggagaccaagaagaacctcacccggaagatccgcagcgaggagctgagcttcacggcggtctccaacgggcccaagaacatcagcggccagtccccggcccggaccagctccgaccccgagaccaacaccacgaacgaggaccacaagatcatggccagcgagaactccagcgccatggtgcaggtgcactcccagggccgcaaggccgcggtcagccacctgaccaccctcgccaccatctccacgagcccccagcccccgaccaccaagaccgggcccgacaactccacgcacaacacccccgtgtacaagctggacatcagcgaggccacccaggtcggccagcaccaccggcgcgccgacaacgactccaccgccagcgacaccccgccggcgacgaccgccgccgggcccctgaaggccgagaacaccaacacctccaagagcgccgactccctcgacctggcgacgaccaccagcccccagaactacagcgagaccgccggcaacaacaacacgcaccaccaggacaccggggaggagtccgccagctccggcaagctgggcctcatcaccaacaccatcgccggggtggcgggcctgatcacgggcgggcgccggacccgccgggaggtgatcgtcaacgcccagcccaagtgcaacccgaacctgcactactggaccacccaggacgagggggccgccatcggcctcgcctggatcccctacttcggccccgcggccgaggggatctacacggagggcctgatgcacaaccaggacgggctgatctgcggcctccgccagctggccaacgagaccacccaggccctgcagctcttcctgcgggccaccacggagctgcgcaccttcagcatcctcaaccggaaggcgatcgacttcctgctgcagcgctggggcgggacctgccacatcctgggcccggactgctgcatcgagccccacgactggaccaagaacatcacggacaagatcgaccagatcatccacgacttcgtggacaagaccctccccgaccagggggacaacgacaactggtggaccggctggcggcagtggatccccgccgggatcggcgtgaccggcgtcatcatcgccgtgatcgccctgttctgcatctgcaagttcgtgttctgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 39 - DNA, 2428 bp, synthetic construct
  misc_feature 1..2428: note = MARV GP, Angola 2005, optimized nucleotide sequence
  source 1..2428: mol_type = other DNA
ggggcgctgcctacggaggtggcagccatctccttctcggcatcaagcttaccatgaagaccacctgcctgctcatcagcctgatcctgatccagggcgtgaagacgctccccatcctggagatcgcctccaacatccagccccagaacgtcgacagcgtgtgctccgggaccctgcagaagaccgaggacgtgcacctcatgggcttcaccctgagcgggcagaaggtcgccgactccccgctggaggcgagcaagcgctgggccttccgggccggcgtgccgcccaagaacgtggagtacacggagggggaggaggccaagacctgctacaacatctccgtcaccgaccccagcggcaagtccctcctgctggacccgcccaccaacatccgcgactaccccaagtgcaagacgatccaccacatccagggccagaacccgcacgcccaggggatcgcgctccacctgtggggcgccttcttcctgtacgaccggatcgccagcaccaccatgtaccgcgggaaggtgttcaccgagggcaacatcgccgcgatgatcgtgaacaagacggtccacaagatgatcttctcccggcaggggcagggctaccgccacatgaacctcaccagcaccaacaagtactggacctccagcaacggcacgcagaccaacgacaccgggtgcttcggcaccctgcaggagtacaactccacgaagaaccagacctgcgcccccagcaagaagcccctgcccctcccgaccgcccaccccgaggtgaagctgacctccacgagcaccgacgccaccaagctgaacaccacggaccccaactccgacgacgaggacctcaccaccagcgggagcggctccggcgagcaggagccctacaccacgagcgacgccgcgaccaagcaggggctgtccagcaccatgccgcccaccccgtccccgcagcccagcacgccccagcagggcgggaacaacaccaaccactcccagggcgtggtcaccgagcccgggaagaccaacaccacggcccagcccagcatgccgccccacaacaccaccaccatctccacgaacaacaccagcaagcacaacctgtccacccccagcgtgcccatccagaacgccaccaactacaacacgcagtccaccgccccggagaacgagcagaccagcgccccctccaagaccacgctcctgcccaccgagaacccgaccaccgcgaagagcacgaactccaccaagagccccaccaccacggtgcccaacaccaccaacaagtactccaccagccccagcccgacgcccaactccaccgcccagcacctggtctacttccggcgcaagcggaacatcctctggcgcgagggcgacatgttccccttcctggacggcctgatcaacgcccccatcgacttcgacccggtgcccaacaccaagaccatcttcgacgagagctccagctccggggccagcgccgaggaggaccagcacgcgtcccccaacatcagcctcacgctgtcctacttccccaaggtgaacgagaacaccgcccacagcggcgagaacgagaacgactgcgacgccgagctgcggatctggtccgtccaggaggacgacctcgccgccgggctgagctggatcccgttcttcggccccgggatcgagggcctgtacaccgcgggcctcatcaagaaccagaacaacctggtgtgccgcctgcggcgcctcgccaaccagaccgccaagtccctggagctgctcctgcgggtgacgaccgaggagcgcaccttcagcctgatcaaccggcacgccatcgacttcctcctggcgcgctggggcgggacctgcaaggtcctggggcccgactgctgcatcggcatcgaggacctgtcccggaacatcagcgagcagatcgaccagatcaagaaggacgagcagaaggaggggacgggctggggcctcgggggcaagtggtggacctccgactggggcgtgctgaccaacctggggatcctcctgctgctcagcatcgccgtgctgatcgccctgagctgcatctgccgcatcttcaccaagtacatcggctgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 40 - DNA, 1363 bp, synthetic construct
  misc_feature 1..1363: note = EBOV VP40, Mayinga, Zaire 1976, optimized nucleotide sequence
  source 1..1363: mol_type = other DNA
ggggcgctgcctacggaggtggcagccatctccttctcggcatcaagcttaccatgcgccgggtgatcctgcccaccgccccgcccgagtacatggaggccatctaccccgtccgcagcaactccaccatcgcgcggggcgggaacagcaacacgggcttcctcacccccgagtccgtgaacggggacaccccgagcaaccccctgcgccccatcgccgacgacaccatcgaccacgcctcccacacgcccggcagcgtgtccagcgccttcatcctggaggccatggtcaacgtgatctccgggccgaaggtgctcatgaagcagatccccatctggctgcccctgggcgtcgcggaccagaagacctacagcttcgactccaccaccgccgccatcatgctcgccagctacacgatcacccacttcggcaaggcgaccaaccccctggtgcgggtgaaccgcctggggccgggcatccccgaccaccccctccggctgctgcgcatcgggaaccaggccttcctccaggagttcgtcctgcccccggtgcagctgccccagtacttcaccttcgacctcacggccctgaagctgatcacccagcccctccccgccgccacctggaccgacgacacgccgaccggctccaacggggcgctgcggcccggcatcagcttccaccccaagctgcgccccatcctcctgccgaacaagtccggcaagaaggggaacagcgccgacctgacctcccccgagaagatccaggccatcatgaccagcctccaggacttcaagatcgtgcccatcgaccccacgaagaacatcatgggcatcgaggtcccggagaccctggtgcacaagctgaccgggaagaaggtgacctccaagaacggccagcccatcatccccgtcctcctgccgaagtacatcggcctggaccccgtggcccccggggacctcacgatggtgatcacccaggactgcgacacctgccacagccccgccagcctgccggcggtcatcgagaagtgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 41 - DNA, 1363 bp, synthetic construct
  misc_feature 1..1363: note = EBOV VP40, Sierra Leone 2014, optimized nucleotide sequence
  source 1..1363: mol_type = other DNA
ggggcgctgcctacggaggtggcagccatctccttctcggcatcaagcttaccatgcgccgggtgatcctgcccaccgccccgcccgagtacatggaggccatctaccccgcgcgcagcaactccaccatcgcccggggcgggaacagcaacacgggcttcctcacccccgagtccgtcaacggggacaccccgagcaaccccctgcgccccatcgccgacgacaccatcgaccacgcctcccacacgcccggcagcgtgtccagcgccttcatcctggaggcgatggtgaacgtcatctccgggccgaaggtgctcatgaagcagatccccatctggctgcccctgggcgtggccgaccagaagacctacagcttcgactccaccaccgccgccatcatgctcgcgagctacacgatcacccacttcggcaaggccaccaaccccctggtccgggtgaaccgcctggggccgggcatccccgaccaccccctccggctgctgcgcatcgggaaccaggccttcctccaggagttcgtgctgcccccggtccagctgccccagtacttcaccttcgacctcacggccctgaagctgatcacccagcccctccccgccgcgacctggaccgacgacacgccgaccggctccaacggggccctgcggcccggcatcagcttccaccccaagctgcgccccatcctcctgccgaacaagtccggcaagaaggggaacagcgccgacctgacctcccccgagaagatccaggccatcatgaccagcctccaggacttcaagatcgtgcccatcgaccccacgaagaacatcatgggcatcgaggtgccggagaccctggtccacaagctgaccgggaagaaggtgacctccaagaacggccagcccatcatccccgtgctcctgccgaagtacatcggcctggaccccgtcgcccccggggacctcacgatggtgatcacccaggactgcgacacctgccacagccccgcgagcctgccggccgtggtcgagaagtgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 42 - DNA, 1294 bp, synthetic construct
  misc_feature 1..1294: note = MARV VP40, Angola 2005, optimized nucleotide sequence
  source 1..1294: mol_type = other DNA
ggggcgctgcctacggaggtggcagccatctccttctcggcatcaagcttaccatggccagctccagcaactacaacacctacatgcagtacctgaacccgccgccctacgccgaccacggcgcgaaccagctcatccccgccgaccagctgtccaaccagcaggggatcacccccaactacgtgggcgacctgaacctcgacgaccagttcaaggggaacgtctgccacgccttcacgctggaggccatcatcgacatcagcgcctacaacgagcgcaccgtgaagggcgtgccggcgtggctgcccctcgggatcatgtccaacttcgagtaccccctggcccacaccgtcgccgccctgctcaccggcagctacacgatcacccagttcacccacaacggccagaagttcgtgcgggtgaaccgcctggggaccggcatccccgcgcacccgctgcggatgctccgcgaggggaaccaggccttcatccagaacatggtcatcccccggaacttctccacgaaccagttcacctacaacctgaccaacctggtgctcagcgtgcagaagctgcccgacgacgcctggcgcccctccaaggacaagctgatcggcaacaccatgcaccccgccgtcagcgtgcaccccaacctcccgcccatcgtgctgccgacggtcaagaagcaggcctaccggcagcacaagaaccccaacaacgggcccctgctcgcgatctccggcatcctgcaccagctgcgcgtggagaaggtgcccgagaagaccagcctcttccggatctccctgccggccgacatgttcagcgtcaaggagggcatgatgaagaagcgcggggagaactcccccgtggtgtacttccaggcccccgagaacttccccctgaacggcttcaacaaccggcaggtcgtgctcgcctacgccaacccgaccctgagcgcggtgtgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 43 - DNA, 2602 bp, synthetic construct
  misc_feature 1..2602: note = EBOV NP, Zaire 1976, optimized nucleotide sequence
  source 1..2602: mol_type = other DNA
ggggcgctgcctacggaggtggcagccatctccttctcggcatcaagcttaccatggacagccgcccccagaagatctggatggccccgtccctgaccgagagcgacatggactaccacaagatcctcaccgccggcctgtccgtgcagcaggggatcgtccggcagcgcgtgatccccgtgtaccaggtcaacaacctggaggagatctgccagctcatcatccaggcgttcgaggccggcgtggacttccaggagagcgccgactccttcctgctgatgctctgcctgcaccacgcctaccagggggactacaagctgttcctcgagagcggcgccgtgaagtacctggaggggcacggcttccggttcgaggtcaagaagcgcgacggcgtgaagcggctggaggagctcctgcccgcggtgtccagcgggaagaacatcaagcgcacgctggccgccatgcccgaggaggagaccaccgaggccaacgcgggccagttcctctccttcgccagcctgttcctgccgaagctcgtcgtgggggagaaggcctgcctggagaaggtgcagcggcagatccaggtccacgccgagcagggcctgatccagtaccccaccgcctggcagtccgtggggcacatgatggtgatcttccgcctcatgcggacgaacttcctgatcaagttcctgctcatccaccagggcatgcacatggtcgcgggccacgacgccaacgacgccgtgatcagcaactccgtggcccaggcccgcttcagcgggctgctgatcgtcaagaccgtgctcgaccacatcctgcagaagaccgagcggggcgtgcgcctgcaccccctcgcgcggaccgccaaggtcaagaacgaggtgaactccttcaaggccgccctgagctccctggccaagcacggggagtacgcgcccttcgcccgcctcctgaacctgagcggcgtgaacaacctcgagcacggcctgttcccgcagctgtccgccatcgccctcggggtcgccacggcgcacggcagcaccctggccggggtgaacgtcggcgagcagtaccagcagctgcgggaggccgccaccgaggcggagaagcagctccagcagtacgccgagagccgcgagctggaccacctggggctcgacgaccaggagaagaagatcctgatgaacttccaccagaagaagaacgagatctccttccagcagaccaacgccatggtgacgctgcggaaggagcgcctggccaagctcaccgaggccatcaccgcggccagcctgcccaagacctccggccactacgacgacgacgacgacatccccttccccggcccgatcaacgacgacgacaaccccgggcaccaggacgacgaccccacggacagccaggacaccaccatccccgacgtggtcgtggacccggacgacggctcctacggggagtaccagagctactccgagaacggcatgaacgcccccgacgacctggtgctcttcgacctggacgaggacgacgaggacaccaagcccgtccccaaccggagcacgaagggcgggcagcagaagaactcccagaagggccagcacatcgaggggcgccagacccagagccggccgatccagaacgtgcccggcccccaccgcaccatccaccacgcctccgccccgctgaccgacaacgaccgccggaacgagcccagcgggtccacgagcccccgcatgctcacccccatcaacgaggaggcggaccccctggacgacgccgacgacgagacctccagcctgccgcccctcgagtccgacgacgaggagcaggaccgggacggcaccagcaaccgcacgcccaccgtggccccgcccgcccccgtctaccgggaccactccgagaagaaggagctgccccaggacgagcagcaggaccaggaccacacccaggaggcccgcaaccaggacagcgacaacacccagagcgagcactccttcgaggagatgtaccggcacatcctgcgcagccaggggccgttcgacgcggtgctctactaccacatgatgaaggacgagcccgtggtcttctccacgagcgacggcaaggagtacacctaccccgactccctggaggaggagtacccgccgtggctgaccgagaaggaggccatgaacgaggagaaccggttcgtgaccctcgacggccagcagttctactggcccgtgatgaaccacaagaacaagttcatggccatcctgcagcaccaccagtgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 44 - DNA, 2602 bp, synthetic construct
  misc_feature 1..2602: note = EBOV NP, Sierra Leone 2014, optimized nucleotide sequence
  source 1..2602: mol_type = other DNA
ggggcgctgcctacggaggtggcagccatctccttctcggcatcaagcttaccatggacagccgcccccagaaggtgtggatgaccccgtccctgaccgagagcgacatggactaccacaagatcctcacggccggcctgtccgtccagcaggggatcgtgcggcagcgcgtgatccccgtctaccaggtgaacaacctggaggagatctgccagctcatcatccaggccttcgaggcgggcgtggacttccaggagagcgccgactccttcctgctgatgctctgcctgcaccacgcctaccagggggactacaagctgttcctcgagagcggcgccgtcaagtacctggaggggcacggcttccggttcgaggtgaagaagtgcgacggcgtgaagcgcctggaggagctcctgcccgccgtctccagcgggcggaacatcaagcgcaccctggcggccatgcccgaggaggagaccaccgaggccaacgccggccagttcctctccttcgcgagcctgttcctgccgaagctcgtggtgggggagaaggcctgcctggagaaggtccagcggcagatccaggtgcacgccgagcagggcctgatccagtaccccacggcctggcagtccgtggggcacatgatggtcatcttccgcctcatgcggaccaacttcctgatcaagttcctgctcatccaccagggcatgcacatggtggccggccacgacgcgaacgacgccgtgatcagcaactccgtcgcccaggcccgcttcagcgggctgctgatcgtgaagaccgtgctcgaccacatcctgcagaagaccgagcggggcgtccgcctgcaccccctcgcccggacggcgaaggtgaagaacgaggtgaactccttcaaggccgccctgagctccctggccaagcacggggagtacgcccccttcgcgcgcctcctgaacctgagcggcgtcaacaacctcgagcacggcctgttcccgcagctgtccgccatcgccctcggggtggccaccgcccacggcagcaccctggcgggggtcaacgtgggcgagcagtaccagcagctgcgggaggccgccaccgaggccgagaagcagctccagcagtacgcggagagccgcgagctggaccacctggggctcgacgaccaggagaagaagatcctgatgaacttccaccagaagaagaacgagatctccttccagcagacgaacgccatggtgaccctgcggaaggagcgcctggccaagctcaccgaggccatcaccgccgcgagcctgcccaagacgtccggccactacgacgacgacgacgacatccccttccccggcccgatcaacgacgacgacaaccccgggcaccaggacgacgaccccaccgacagccaggacaccaccatccccgacgtcgtggtggacccggacgacggcgggtacggcgagtaccagtcctacagcgagaacgggatgtccgcccccgacgacctggtcctcttcgacctggacgaggacgacgaggacacgaagcccgtgcccaaccggagcaccaagggcggccagcagaagaactcccagaaggggcagcacaccgagggccgccagacccagagcacgccgacccagaacgtgaccgggccccggcgcaccatccaccacgcctccgccccgctgacggacaacgaccgccggaacgagcccagcggctccaccagcccgcgcatgctcacccccatcaacgaggaggccgaccccctggacgacgcggacgacgagacctccagcctgcccccgctcgagtccgacgacgaggagcaggaccgggacgggacgagcaaccgcacccccaccgtcgccccgcccgcccccgtgtaccgggaccactccgagaagaaggagctgccccaggacgagcagcaggaccaggaccacatccaggaggcccgcaaccaggacagcgacaacacccagcccgagcacagcttcgaggagatgtaccggcacatcctgcgctcccagggcccgttcgacgccgtgctctactaccacatgatgaaggacgagcccgtcgtgttcagcacgtccgacggcaaggagtacacctaccccgacagcctggaggaggagtacccgccgtggctgaccgagaaggaggcgatgaacgacgagaaccggttcgtgaccctcgacgggcagcagttctactggcccgtcatgaaccaccgcaacaagttcatggccatcctgcagcaccaccagtgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 45 - RNA, 2413 nt, synthetic construct
  misc_feature 1..2413: note = EBOV GP, Mayinga, Zaire 1976, optimized nucleotide sequence
  source 1..2413: mol_type = other RNA
ggggcgctgcctacggaggtggcagccatctccttctcggcatcaagcttaccatgggcgtgaccgggatcctgcagctcccccgcgaccggttcaagcgcaccagcttcttcctgtgggtcatcatcctgttccagcggacgttctccatcccgctcggcgtgatccacaacagcaccctgcaggtgtccgacgtcgacaagctggtgtgccgcgacaagctcagctccaccaaccagctgcggagcgtggggctgaacctcgagggcaacggggtcgccaccgacgtgccctccgccacgaagcgctggggcttccggagcggcgtgccgcccaaggtcgtgaactacgaggcgggggagtgggccgagaactgctacaacctggagatcaagaagcccgacggctccgagtgcctgcccgccgcccccgacgggatccgcggcttcccccggtgccgctacgtgcacaaggtcagcgggaccggcccgtgcgccggcgacttcgcgttccacaaggagggggccttcttcctctacgaccggctggcctccaccgtgatctaccgcggcaccacgttcgccgagggggtggtcgcgttcctgatcctcccccaggccaagaaggacttcttcagctcccaccccctgcgggagcccgtgaacgccaccgaggacccgagctccggctactacagcaccaccatccgctaccaggccacgggcttcgggaccaacgagaccgagtacctgttcgaggtggacaacctcacctacgtccagctggagtcccggttcacgccccagttcctgctccagctgaacgagaccatctacaccagcggcaagcgctccaacaccacggggaagctgatctggaaggtgaaccccgagatcgacaccaccatcggcgagtgggccttctgggagaccaagaagaacctcacgcggaagatccgcagcgaggagctgagcttcaccgtggtctccaacggggcgaagaacatcagcggccagtcccccgcccggaccagctccgacccgggcaccaacacgaccaccgaggaccacaagatcatggccagcgagaactccagcgccatggtgcaggtgcactcccaggggcgcgaggccgcggtcagccacctgaccacgctcgccaccatctccaccagcccccagtccctgaccacgaagcccggccccgacaacagcacccacaacaccccggtgtacaagctggacatctccgaggccacccaggtcgagcagcaccaccggcgcaccgacaacgacagcacggcctccgacacccccagcgccaccaccgcggccgggccgcccaaggccgagaacacgaacacctccaagagcaccgacttcctcgaccccgccaccacgaccagcccccagaaccactccgagaccgccggcaacaacaacacccaccaccaggacacgggggaggagagcgcgtccagcggcaagctgggcctgatcaccaacaccatcgccggggtggccggcctcatcaccgggggccgccggacgcgccgggaggccatcgtgaacgcgcagcccaagtgcaaccccaacctgcactactggaccacccaggacgagggggccgccatcggcctggcctggatcccgtacttcggccccgccgcggaggggatctacatcgagggcctcatgcacaaccaggacgggctgatctgcggcctgcgccagctcgccaacgagaccacgcaggccctgcagctgttcctccgggccaccaccgagctgcgcaccttctccatcctgaaccggaaggccatcgacttcctcctgcagcgctggggcgggacgtgccacatcctgggccccgactgctgcatcgagccgcacgactggaccaagaacatcaccgacaagatcgaccagatcatccacgacttcgtcgacaagaccctgcccgaccagggggacaacgacaactggtggacgggctggcggcagtggatccccgcggggatcggcgtgaccggcgtgatcatcgccgtcatcgccctcttctgcatctgcaagttcgtgttctgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 46 - RNA, 2413 nt, synthetic construct
  misc_feature 1..2413: note = EBOV GP, Sierra Leone 2014, optimized nucleotide sequence
  source 1..2413: mol_type = other RNA
ggggcgctgcctacggaggtggcagccatctccttctcggcatcaagcttaccatgggcgtgaccgggatcctgcagctcccccgcgaccggttcaagcgcaccagcttcttcctgtgggtcatcatcctgttccagcggacgttctccatcccgctcggcgtgatccacaacagcaccctgcaggtgtccgacgtcgacaagctggtgtgccgcgacaagctcagctccaccaaccagctgcggagcgtggggctgaacctcgagggcaacggggtcgccaccgacgtgccctccgtgacgaagcgctggggcttccggagcggcgtcccgcccaaggtggtgaactacgaggccggggagtgggcggagaactgctacaacctggagatcaagaagcccgacggctccgagtgcctgcccgccgcccccgacgggatccgcggcttcccccggtgccgctacgtccacaaggtgagcgggaccggcccgtgcgccggcgacttcgccttccacaaggagggggcgttcttcctctacgaccggctggcctccaccgtgatctaccgcggcaccacgttcgccgagggggtcgtggccttcctgatcctcccccaggcgaagaaggacttcttcagctcccaccccctgcgggagcccgtgaacgccaccgaggacccgagctccggctactacagcaccaccatccgctaccaggccacgggcttcgggaccaacgagaccgagtacctgttcgaggtcgacaacctcacctacgtgcagctggagtcccggttcacgccccagttcctgctccagctgaacgagaccatctacgccagcggcaagcgctccaacaccaccgggaagctgatctggaaggtgaaccccgagatcgacacgaccatcggcgagtgggccttctgggagaccaagaagaacctcacccggaagatccgcagcgaggagctgagcttcacggcggtctccaacgggcccaagaacatcagcggccagtccccggcccggaccagctccgaccccgagaccaacaccacgaacgaggaccacaagatcatggccagcgagaactccagcgccatggtgcaggtgcactcccagggccgcaaggccgcggtcagccacctgaccaccctcgccaccatctccacgagcccccagcccccgaccaccaagaccgggcccgacaactccacgcacaacacccccgtgtacaagctggacatcagcgaggccacccaggtcggccagcaccaccggcgcgccgacaacgactccaccgccagcgacaccccgccggcgacgaccgccgccgggcccctgaaggccgagaacaccaacacctccaagagcgccgactccctcgacctggcgacgaccaccagcccccagaactacagcgagaccgccggcaacaacaacacgcaccaccaggacaccggggaggagtccgccagctccggcaagctgggcctcatcaccaacaccatcgccggggtggcgggcctgatcacgggcgggcgccggacccgccgggaggtgatcgtcaacgcccagcccaagtgcaacccgaacctgcactactggaccacccaggacgagggggccgccatcggcctcgcctggatcccctacttcggccccgcggccgaggggatctacacggagggcctgatgcacaaccaggacgggctgatctgcggcctccgccagctggccaacgagaccacccaggccctgcagctcttcctgcgggccaccacggagctgcgcaccttcagcatcctcaaccggaaggcgatcgacttcctgctgcagcgctggggcgggacctgccacatcctgggcccggactgctgcatcgagccccacgactggaccaagaacatcacggacaagatcgaccagatcatccacgacttcgtggacaagaccctccccgaccagggggacaacgacaactggtggaccggctggcggcagtggatccccgccgggatcggcgtgaccggcgtcatcatcgccgtgatcgccctgttctgcatctgcaagttcgtgttctgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 47 - RNA, 2428 nt, synthetic construct
  misc_feature 1..2428: note = MARV GP, Angola 2005, optimized nucleotide sequence
  source 1..2428: mol_type = other RNA
ggggcgctgcctacggaggtggcagccatctccttctcggcatcaagcttaccatgaagaccacctgcctgctcatcagcctgatcctgatccagggcgtgaagacgctccccatcctggagatcgcctccaacatccagccccagaacgtcgacagcgtgtgctccgggaccctgcagaagaccgaggacgtgcacctcatgggcttcaccctgagcgggcagaaggtcgccgactccccgctggaggcgagcaagcgctgggccttccgggccggcgtgccgcccaagaacgtggagtacacggagggggaggaggccaagacctgctacaacatctccgtcaccgaccccagcggcaagtccctcctgctggacccgcccaccaacatccgcgactaccccaagtgcaagacgatccaccacatccagggccagaacccgcacgcccaggggatcgcgctccacctgtggggcgccttcttcctgtacgaccggatcgccagcaccaccatgtaccgcgggaaggtgttcaccgagggcaacatcgccgcgatgatcgtgaacaagacggtccacaagatgatcttctcccggcaggggcagggctaccgccacatgaacctcaccagcaccaacaagtactggacctccagcaacggcacgcagaccaacgacaccgggtgcttcggcaccctgcaggagtacaactccacgaagaaccagacctgcgcccccagcaagaagcccctgcccctcccgaccgcccaccccgaggtgaagctgacctccacgagcaccgacgccaccaagctgaacaccacggaccccaactccgacgacgaggacctcaccaccagcgggagcggctccggcgagcaggagccctacaccacgagcgacgccgcgaccaagcaggggctgtccagcaccatgccgcccaccccgtccccgcagcccagcacgccccagcagggcgggaacaacaccaaccactcccagggcgtggtcaccgagcccgggaagaccaacaccacggcccagcccagcatgccgccccacaacaccaccaccatctccacgaacaacaccagcaagcacaacctgtccacccccagcgtgcccatccagaacgccaccaactacaacacgcagtccaccgccccggagaacgagcagaccagcgccccctccaagaccacgctcctgcccaccgagaacccgaccaccgcgaagagcacgaactccaccaagagccccaccaccacggtgcccaacaccaccaacaagtactccaccagccccagcccgacgcccaactccaccgcccagcacctggtctacttccggcgcaagcggaacatcctctggcgcgagggcgacatgttccccttcctggacggcctgatcaacgcccccatcgacttcgacccggtgcccaacaccaagaccatcttcgacgagagctccagctccggggccagcgccgaggaggaccagcacgcgtcccccaacatcagcctcacgctgtcctacttccccaaggtgaacgagaacaccgcccacagcggcgagaacgagaacgactgcgacgccgagctgcggatctggtccgtccaggaggacgacctcgccgccgggctgagctggatcccgttcttcggccccgggatcgagggcctgtacaccgcgggcctcatcaagaaccagaacaacctggtgtgccgcctgcggcgcctcgccaaccagaccgccaagtccctggagctgctcctgcgggtgacgaccgaggagcgcaccttcagcctgatcaaccggcacgccatcgacttcctcctggcgcgctggggcgggacctgcaaggtcctggggcccgactgctgcatcggcatcgaggacctgtcccggaacatcagcgagcagatcgaccagatcaagaaggacgagcagaaggaggggacgggctggggcctcgggggcaagtggtggacctccgactggggcgtgctgaccaacctggggatcctcctgctgctcagcatcgccgtgctgatcgccctgagctgcatctgccgcatcttcaccaagtacatcggctgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 48 - RNA, 1363 nt, synthetic construct
  misc_feature 1..1363: note = EBOV VP40, Mayinga, Zaire 1976, optimized nucleotide sequence
  source 1..1363: mol_type = other RNA
ggggcgctgcctacggaggtggcagccatctccttctcggcatcaagcttaccatgcgccgggtgatcctgcccaccgccccgcccgagtacatggaggccatctaccccgtccgcagcaactccaccatcgcgcggggcgggaacagcaacacgggcttcctcacccccgagtccgtgaacggggacaccccgagcaaccccctgcgccccatcgccgacgacaccatcgaccacgcctcccacacgcccggcagcgtgtccagcgccttcatcctggaggccatggtcaacgtgatctccgggccgaaggtgctcatgaagcagatccccatctggctgcccctgggcgtcgcggaccagaagacctacagcttcgactccaccaccgccgccatcatgctcgccagctacacgatcacccacttcggcaaggcgaccaaccccctggtgcgggtgaaccgcctggggccgggcatccccgaccaccccctccggctgctgcgcatcgggaaccaggccttcctccaggagttcgtcctgcccccggtgcagctgccccagtacttcaccttcgacctcacggccctgaagctgatcacccagcccctccccgccgccacctggaccgacgacacgccgaccggctccaacggggcgctgcggcccggcatcagcttccaccccaagctgcgccccatcctcctgccgaacaagtccggcaagaaggggaacagcgccgacctgacctcccccgagaagatccaggccatcatgaccagcctccaggacttcaagatcgtgcccatcgaccccacgaagaacatcatgggcatcgaggtcccggagaccctggtgcacaagctgaccgggaagaaggtgacctccaagaacggccagcccatcatccccgtcctcctgccgaagtacatcggcctggaccccgtggcccccggggacctcacgatggtgatcacccaggactgcgacacctgccacagccccgccagcctgccggcggtcatcgagaagtgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 49 - RNA, 1363 nt, synthetic construct
  misc_feature 1..1363: note = EBOV VP40, Sierra Leone 2014, optimized nucleotide sequence
  source 1..1363: mol_type = other RNA
ggggcgctgcctacggaggtggcagccatctccttctcggcatcaagcttaccatgcgccgggtgatcctgcccaccgccccgcccgagtacatggaggccatctaccccgcgcgcagcaactccaccatcgcccggggcgggaacagcaacacgggcttcctcacccccgagtccgtcaacggggacaccccgagcaaccccctgcgccccatcgccgacgacaccatcgaccacgcctcccacacgcccggcagcgtgtccagcgccttcatcctggaggcgatggtgaacgtcatctccgggccgaaggtgctcatgaagcagatccccatctggctgcccctgggcgtggccgaccagaagacctacagcttcgactccaccaccgccgccatcatgctcgcgagctacacgatcacccacttcggcaaggccaccaaccccctggtccgggtgaaccgcctggggccgggcatccccgaccaccccctccggctgctgcgcatcgggaaccaggccttcctccaggagttcgtgctgcccccggtccagctgccccagtacttcaccttcgacctcacggccctgaagctgatcacccagcccctccccgccgcgacctggaccgacgacacgccgaccggctccaacggggccctgcggcccggcatcagcttccaccccaagctgcgccccatcctcctgccgaacaagtccggcaagaaggggaacagcgccgacctgacctcccccgagaagatccaggccatcatgaccagcctccaggacttcaagatcgtgcccatcgaccccacgaagaacatcatgggcatcgaggtgccggagaccctggtccacaagctgaccgggaagaaggtgacctccaagaacggccagcccatcatccccgtgctcctgccgaagtacatcggcctggaccccgtcgcccccggggacctcacgatggtgatcacccaggactgcgacacctgccacagccccgcgagcctgccggccgtggtcgagaagtgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 50 - RNA, 1294 nt, synthetic construct
  misc_feature 1..1294: note = MARV VP40, Angola 2005, optimized nucleotide sequence
  source 1..1294: mol_type = other RNA
ggggcgctgcctacggaggtggcagccatctccttctcggcatcaagcttaccatggccagctccagcaactacaacacctacatgcagtacctgaacccgccgccctacgccgaccacggcgcgaaccagctcatccccgccgaccagctgtccaaccagcaggggatcacccccaactacgtgggcgacctgaacctcgacgaccagttcaaggggaacgtctgccacgccttcacgctggaggccatcatcgacatcagcgcctacaacgagcgcaccgtgaagggcgtgccggcgtggctgcccctcgggatcatgtccaacttcgagtaccccctggcccacaccgtcgccgccctgctcaccggcagctacacgatcacccagttcacccacaacggccagaagttcgtgcgggtgaaccgcctggggaccggcatccccgcgcacccgctgcggatgctccgcgaggggaaccaggccttcatccagaacatggtcatcccccggaacttctccacgaaccagttcacctacaacctgaccaacctggtgctcagcgtgcagaagctgcccgacgacgcctggcgcccctccaaggacaagctgatcggcaacaccatgcaccccgccgtcagcgtgcaccccaacctcccgcccatcgtgctgccgacggtcaagaagcaggcctaccggcagcacaagaaccccaacaacgggcccctgctcgcgatctccggcatcctgcaccagctgcgcgtggagaaggtgcccgagaagaccagcctcttccggatctccctgccggccgacatgttcagcgtcaaggagggcatgatgaagaagcgcggggagaactcccccgtggtgtacttccaggcccccgagaacttccccctgaacggcttcaacaaccggcaggtcgtgctcgcctacgccaacccgaccctgagcgcggtgtgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 51 - RNA, 2602 nt, synthetic construct
  misc_feature 1..2602: note = EBOV NP, Zaire 1976, optimized nucleotide sequence
  source 1..2602: mol_type = other RNA
ggggcgctgcctacggaggtggcagccatctccttctcggcatcaagcttaccatggacagccgcccccagaagatctggatggccccgtccctgaccgagagcgacatggactaccacaagatcctcaccgccggcctgtccgtgcagcaggggatcgtccggcagcgcgtgatccccgtgtaccaggtcaacaacctggaggagatctgccagctcatcatccaggcgttcgaggccggcgtggacttccaggagagcgccgactccttcctgctgatgctctgcctgcaccacgcctaccagggggactacaagctgttcctcgagagcggcgccgtgaagtacctggaggggcacggcttccggttcgaggtcaagaagcgcgacggcgtgaagcggctggaggagctcctgcccgcggtgtccagcgggaagaacatcaagcgcacgctggccgccatgcccgaggaggagaccaccgaggccaacgcgggccagttcctctccttcgccagcctgttcctgccgaagctcgtcgtgggggagaaggcctgcctggagaaggtgcagcggcagatccaggtccacgccgagcagggcctgatccagtaccccaccgcctggcagtccgtggggcacatgatggtgatcttccgcctcatgcggacgaacttcctgatcaagttcctgctcatccaccagggcatgcacatggtcgcgggccacgacgccaacgacgccgtgatcagcaactccgtggcccaggcccgcttcagcgggctgctgatcgtcaagaccgtgctcgaccacatcctgcagaagaccgagcggggcgtgcgcctgcaccccctcgcgcggaccgccaaggtcaagaacgaggtgaactccttcaaggccgccctgagctccctggccaagcacggggagtacgcgcccttcgcccgcctcctgaacctgagcggcgtgaacaacctcgagcacggcctgttcccgcagctgtccgccatcgccctcggggtcgccacggcgcacggcagcaccctggccggggtgaacgtcggcgagcagtaccagcagctgcgggaggccgccaccgaggcggagaagcagctccagcagtacgccgagagccgcgagctggaccacctggggctcgacgaccaggagaagaagatcctgatgaacttccaccagaagaagaacgagatctccttccagcagaccaacgccatggtgacgctgcggaaggagcgcctggccaagctcaccgaggccatcaccgcggccagcctgcccaagacctccggccactacgacgacgacgacgacatccccttccccggcccgatcaacgacgacgacaaccccgggcaccaggacgacgaccccacggacagccaggacaccaccatccccgacgtggtcgtggacccggacgacggctcctacggggagtaccagagctactccgagaacggcatgaacgcccccgacgacctggtgctcttcgacctggacgaggacgacgaggacaccaagcccgtccccaaccggagcacgaagggcgggcagcagaagaactcccagaagggccagcacatcgaggggcgccagacccagagccggccgatccagaacgtgcccggcccccaccgcaccatccaccacgcctccgccccgctgaccgacaacgaccgccggaacgagcccagcgggtccacgagcccccgcatgctcacccccatcaacgaggaggcggaccccctggacgacgccgacgacgagacctccagcctgccgcccctcgagtccgacgacgaggagcaggaccgggacggcaccagcaaccgcacgcccaccgtggccccgcccgcccccgtctaccgggaccactccgagaagaaggagctgccccaggacgagcagcaggaccaggaccacacccaggaggcccgcaaccaggacagcgacaacacccagagcgagcactccttcgaggagatgtaccggcacatcctgcgcagccaggggccgttcgacgcggtgctctactaccacatgatgaaggacgagcccgtggtcttctccacgagcgacggcaaggagtacacctaccccgactccctggaggaggagtacccgccgtggctgaccgagaaggaggccatgaacgaggagaaccggttcgtgaccctcgacggccagcagttctactggcccgtgatgaaccacaagaacaagttcatggccatcctgcagcaccaccagtgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 52 - RNA, 2602 nt, synthetic construct
  misc_feature 1..2602: note = EBOV NP, Sierra Leone 2014, optimized nucleotide sequence
  source 1..2602: mol_type = other RNA
ggggcgctgcctacggaggtggcagccatctccttctcggcatcaagcttaccatggacagccgcccccagaaggtgtggatgaccccgtccctgaccgagagcgacatggactaccacaagatcctcacggccggcctgtccgtccagcaggggatcgtgcggcagcgcgtgatccccgtctaccaggtgaacaacctggaggagatctgccagctcatcatccaggccttcgaggcgggcgtggacttccaggagagcgccgactccttcctgctgatgctctgcctgcaccacgcctaccagggggactacaagctgttcctcgagagcggcgccgtcaagtacctggaggggcacggcttccggttcgaggtgaagaagtgcgacggcgtgaagcgcctggaggagctcctgcccgccgtctccagcgggcggaacatcaagcgcaccctggcggccatgcccgaggaggagaccaccgaggccaacgccggccagttcctctccttcgcgagcctgttcctgccgaagctcgtggtgggggagaaggcctgcctggagaaggtccagcggcagatccaggtgcacgccgagcagggcctgatccagtaccccacggcctggcagtccgtggggcacatgatggtcatcttccgcctcatgcggaccaacttcctgatcaagttcctgctcatccaccagggcatgcacatggtggccggccacgacgcgaacgacgccgtgatcagcaactccgtcgcccaggcccgcttcagcgggctgctgatcgtgaagaccgtgctcgaccacatcctgcagaagaccgagcggggcgtccgcctgcaccccctcgcccggacggcgaaggtgaagaacgaggtgaactccttcaaggccgccctgagctccctggccaagcacggggagtacgcccccttcgcgcgcctcctgaacctgagcggcgtcaacaacctcgagcacggcctgttcccgcagctgtccgccatcgccctcggggtggccaccgcccacggcagcaccctggcgggggtcaacgtgggcgagcagtaccagcagctgcgggaggccgccaccgaggccgagaagcagctccagcagtacgcggagagccgcgagctggaccacctggggctcgacgaccaggagaagaagatcctgatgaacttccaccagaagaagaacgagatctccttccagcagacgaacgccatggtgaccctgcggaaggagcgcctggccaagctcaccgaggccatcaccgccgcgagcctgcccaagacgtccggccactacgacgacgacgacgacatccccttccccggcccgatcaacgacgacgacaaccccgggcaccaggacgacgaccccaccgacagccaggacaccaccatccccgacgtcgtggtggacccggacgacggcgggtacggcgagtaccagtcctacagcgagaacgggatgtccgcccccgacgacctggtcctcttcgacctggacgaggacgacgaggacacgaagcccgtgcccaaccggagcaccaagggcggccagcagaagaactcccagaaggggcagcacaccgagggccgccagacccagagcacgccgacccagaacgtgaccgggccccggcgcaccatccaccacgcctccgccccgctgacggacaacgaccgccggaacgagcccagcggctccaccagcccgcgcatgctcacccccatcaacgaggaggccgaccccctggacgacgcggacgacgagacctccagcctgcccccgctcgagtccgacgacgaggagcaggaccgggacgggacgagcaaccgcacccccaccgtcgccccgcccgcccccgtgtaccgggaccactccgagaagaaggagctgccccaggacgagcagcaggaccaggaccacatccaggaggcccgcaaccaggacagcgacaacacccagcccgagcacagcttcgaggagatgtaccggcacatcctgcgctcccagggcccgttcgacgccgtgctctactaccacatgatgaaggacgagcccgtcgtgttcagcacgtccgacggcaaggagtacacctaccccgacagcctggaggaggagtacccgccgtggctgaccgagaaggaggcgatgaacgacgagaaccggttcgtgaccctcgacgggcagcagttctactggcccgtcatgaaccaccgcaacaagttcatggccatcctgcagcaccaccagtgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 53 - RNA, 2031 nt, synthetic construct
  misc_feature 1..2031: note = EBOV GP, Mayinga, Zaire 1976, CDS mRNA wild type
  source 1..2031: mol_type = other RNA
atgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttag

SEQ ID NO: 54 - RNA, 2031 nt, synthetic construct
  misc_feature 1..2031: note = EBOV GP, Sierra Leone 2014, CDS mRNA wild type
  source 1..2031: mol_type = other RNA
atgggtgttacaggaatattgcagttacctcgtgatcgattcaagaggacatcattctttctttgggtaattatccttttccaaagaacattttccatcccgcttggagttatccacaatagtacattacaggttagtgatgtcgacaaactagtttgtcgtgacaaactgtcatccacaaatcaattgagatcagttggactgaatctcgaggggaatggagtggcaactgacgtgccatctgtgactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccatgtgccggagactttgccttccacaaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtcgagtggctattattctaccacaattagatatcaggctaccggttttggaactaatgagacagagtacttgttcgaggttgacaatttgacctacgtccaacttgaatcaagattcacaccacagtttctgctccagctgaatgagacaatatatgcaagtgggaagaggagcaacaccacgggaaaactaatttggaaggtcaaccccgaaattgatacaacaatcggggagtgggccttctgggaaactaaaaaaaacctcactagaaaaattcgcagtgaagagttgtctttcacagctgtatcaaacggacccaaaaacatcagtggtcagagtccggcgcgaacttcttccgacccagagaccaacacaacaaatgaagaccacaaaatcatggcttcagaaaattcctctgcaatggttcaagtgcacagtcaaggaaggaaagctgcagtgtcgcatctgacaacccttgccacaatctccacgagtcctcaacctcccacaaccaaaacaggtccggacaacagcacccataatacacccgtgtataaacttgacatctctgaggcaactcaagttggacaacatcaccgtagagcagacaacgacagcacagcctccgacactccccccgccacgaccgcagccggacccttaaaagcagagaacaccaacacgagtaagagcgctgactccctggacctcgccaccacgacaagcccccaaaactacagcgagactgctggcaacaacaacactcatcaccaagataccggagaagagagtgccagcagcgggaagctaggcttaattaccaatactattgctggagtagcaggactgatcacaggcgggagaaggactcgaagagaagtaattgtcaatgctcaacccaaatgcaaccccaatttacattactggactactcaggatgaaggtgctgcaatcggattggcctggataccatatttcgggccagcagccgaaggaatttacacagaggggctaatgcacaaccaagatggtttaatctgtgggttgaggcagctggccaacgaaacgactcaagctctccaactgttcctgagagccacaactgagctgcgaaccttttcaatcctcaaccgtaaggcaattgacttcctgctgcagcgatggggtggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggtgttataattgcagttatcgctttattctgtatatgcaaatttgtcttttag

SEQ ID NO: 55 - RNA, 2046 nt, synthetic construct
  misc_feature 1..2046: note = MARV GP, Angola 2005, CDS mRNA wild type
  source 1..2046: mol_type = other RNA
atgaaaaccacatgtctccttatcagtcttatcttaatccaaggggtaaaaactctccctattttagagatagccagtaacattcaaccccaaaatgtggattcagtatgctccgggactctccagaagacagaagacgttcatctgatgggattcacactgagcgggcaaaaagttgctgattcccctttagaggcatccaaacgatgggccttcagggcaggtgtacctcccaagaatgttgagtatacagaaggggaggaagctaaaacatgttacaatataagtgtaacggatccctctggaaaatccttgctgttagatcctcctaccaacatccgtgactatcctaaatgcaaaactatccatcatattcaaggtcaaaaccctcatgcacaggggatcgctctccatttgtggggagcatttttcttgtatgatcgcatcgcctccacaacgatgtatcgaggcaaagtcttcactgaagggaacatagcagctatgattgtcaataagacagtgcacaaaatgattttctcgaggcaaggacaagggtaccgtcacatgaacctaacttctactaataaatattggacaagtagcaacggaacgcaaacgaatgacactggatgcttcggtactcttcaagaatataattctacaaagaaccaaacatgtgctccgtccaaaaaacctttaccactgcccacagcccatccggaggtcaagctcactagcacctcaactgatgccaccaaactcaataccacagacccaaacagtgatgatgaggacctcacaacatctggctcagggtctggagaacaggaaccttacacaacttctgacgcagccacgaagcaagggctttcatcaacaatgccgcccactccctcaccacaaccaagcacgccacagcaaggaggaaacaacacgaaccattcccaaggtgttgtgactgaacccggcaaaaccaacacaactgcacaaccgtccatgccccctcacaacactactacaatctctactaacaacacctccaagcacaacctcagcactccctctgtaccaatacaaaatgccactaattacaacacacagagcacggcccctgaaaatgagcaaaccagtgccccctcgaaaacaaccctgcttccaacagaaaatcctacaacagcaaagagcaccaatagtacaaaaagccccactacaacagtaccaaatacgacaaataagtattccaccagtccctcccccacccccaactcgactgcacaacatcttgtatatttcagaaggaaacgaaatattctctggagggaaggcgacatgttcccttttctggatgggttaataaatgctccgattgattttgatccggttccaaatacaaagacaatctttgatgaatcctctagttctggtgcttcagctgaggaagatcagcatgcctcccctaatatcagtttaactttatcttactttcctaaggtaaatgaaaacactgcccactctggagaaaatgaaaatgattgtgatgcagagttaagaatttggagtgttcaggaggacgacctggcagcaggactcagttggataccgttttttggccctggaatcgaaggactttatactgctggtttaattaaaaatcaaaataatttggtttgcaggttgaggcgtctagccaatcagactgccaaatccttggaactcttattaagagtcacaaccgaggaaagaacattttccttaatcaatagacatgccattgattttttactcgcaaggtggggaggaacatgcaaagtgcttggacctgattgttgcatcggaatagaagacttgtccagaaatatttcagaacaaattgatcaaatcaaaaaggacgaacaaaaagaggggactggttggggtctgggtggtaaatggtggacatcagactggggtgttcttactaacttgggcatcttgctactactgtccatagctgtcttaattgctctgtcctgtatttgtcgtatttttactaaatatattggataa

SEQ ID NO: 56 - RNA, 981 nt, synthetic construct
  misc_feature 1..981: note = EBOV VP40, Mayinga, Zaire 1976, CDS mRNA wild type
  source 1..981: mol_type = other RNA
atgaggcgggttatattgcctactgctcctcctgaatatatggaggccatataccctgtcaggtcaaattcaacaattgctagaggtggcaacagcaatacaggcttcctgacaccggagtcagtcaatggggacactccatcgaatccactcaggccaattgccgatgacaccatcgaccatgccagccacacaccaggcagtgtgtcatcagcattcatccttgaagctatggtgaatgtcatatcgggccccaaagtgctaatgaagcaaattccaatttggcttcctctaggtgtcgctgatcaaaagacctacagctttgactcaactacggccgccatcatgcttgcttcatacactatcacccatttcggcaaggcaaccaatccacttgtcagagtcaatcggctgggtcctggaatcccggatcatcccctcaggctcctgcgaattggaaaccaggctttcctccaggagttcgttcttccgccagtccaactaccccagtatttcacctttgatttgacagcactcaaactgatcacccaaccactgcctgctgcaacatggaccgatgacactccaacaggatcaaatggagcgttgcgtccaggaatttcatttcatccaaaacttcgccccattcttttacccaacaaaagtgggaagaaggggaacagtgccgatctaacatctccggagaaaatccaagcaataatgacttcactccaggactttaagatcgttccaattgatccaaccaaaaatatcatgggaatcgaagtgccagaaactctggtccacaagctgaccggtaagaaggtgacttctaaaaatggacaaccaatcatccctgttcttttgccaaagtacattgggttggacccggtggctccaggagacctcaccatggtaatcacacaggattgtgacacgtgtcattctcctgcaagtcttccagctgtgattgagaagtaa

SEQ ID NO: 57 - RNA, 981 nt, synthetic construct
  misc_feature 1..981: note = EBOV VP40, Sierra Leone 2014, CDS mRNA wild type
  source 1..981: mol_type = other RNA
atgaggcgggttatattgcctactgctcctcctgaatatatggaggccatataccctgccaggtcaaattcaacaattgctaggggtggcaacagcaatacaggcttcctgacaccggagtcagtcaatggagacactccatcgaatccactcaggccaattgctgatgacaccatcgaccatgccagccacacaccaggcagtgtgtcatcagcattcatcctcgaagctatggtgaatgtcatatcgggccccaaagtgctaatgaagcaaattccaatttggcttcctctaggtgtcgctgatcaaaagacctacagctttgactcaactacggccgccatcatgcttgcttcatatactatcacccatttcggcaaggcaaccaatccgcttgtcagagtcaatcggctgggtcctggaatcccggatcaccccctcaggctcctgcgaattggaaaccaggctttcctccaggagttcgttcttccaccagtccaactaccccagtatttcacctttgatttgacagcactcaaactgatcactcaaccactgcctgctgcaacatggaccgatgacactccaactggatcaaatggagcgttgcgtccaggaatttcatttcatccaaaacttcgccccattcttttacccaacaaaagtgggaagaaggggaacagtgccgatctaacatctccggagaaaatccaagcaataatgacttcactccaggactttaagatcgttccaattgatccaaccaaaaatatcatgggtatcgaagtgccagaaactctggtccacaagctgaccggtaagaaggtgacttccaaaaatggacaaccaatcatccctgttcttttgccaaagtacattgggttggacccggtggctccaggagacctcaccatggtaatcacacaggattgtgacacgtgtcattctcctgcaagtcttccagctgtggttgagaagtaa

SEQ ID NO: 58 - RNA, 912 nt, synthetic construct
  misc_feature 1..912: note = MARV VP40, Angola 2005, CDS mRNA wild type
  source 1..912: mol_type = other RNA
atggccagttccagcaattacaatacatacatgcaataccttaacccccctccttatgctgaccacggtgcaaaccagttaatcccggcggatcagctatcaaatcagcagggtataactccaaattatgtgggtgatttaaacctagatgaccagttcaaagggaatgtctgccatgctttcactttagaggcaataattgacatatctgcgtataacgaacgaacagtcaaaggcgttccggcatggctgcctcttgggatcatgagcaatttcgaatatcctttagcccatacagtggctgcgttgctcacaggcagctatacaatcacccagtttactcataatgggcaaaaattcgtccgtgtcaatcgactcggtacaggaatcccggcacacccactcaggatgttgcgtgaaggaaatcaagcttttattcagaatatggtgatccccaggaatttttccaccaatcaattcacctacaatctcactaacttagtattgagtgtgcaaaaacttcctgatgatgcctggcgtccgtccaaggacaaattaattggaaacaccatgcatcctgcagtctccgttcacccgaatttaccgcctattgttctaccaacagtcaagaagcaggcttatcgccagcacaaaaatcccaacaatggtccactgctggccatatctggcatccttcatcaactgagagtcgaaaaagtcccagaaaagacaagcctgtttaggatttcgcttcctgccgacatgttctcagtaaaagagggtatgatgaagaaaagaggagaaaattccccggtagtttattttcaagcacctgagaacttccctttgaatggcttcaacaacagacaagttgtactagcgtatgcgaatccaacactcagcgccgtttaa

SEQ ID NO: 59 - RNA, 2220 nt, synthetic construct
  misc_feature 1..2220: note = EBOV NP, Zaire 1976, CDS mRNA wild type
  source 1..2220: mol_type = other RNA
atggattctcgtcctcagaaaatctggatggcgccgagtctcactgaatctgacatggattaccacaagatcttgacagcaggtctgtccgttcaacaggggattgttcggcaaagagtcatcccagtgtatcaagtaaacaatcttgaagaaatttgccaacttatcatacaggcctttgaagcaggtgttgattttcaagagagtgcggacagtttccttctcatgctttgtcttcatcatgcgtaccagggagattacaaacttttcttggaaagtggcgcagtcaagtatttggaagggcacgggttccgttttgaagtcaagaagcgtgatggagtgaagcgccttgaggaattgctgccagcagtatctagtggaaaaaacattaagagaacacttgctgccatgccggaagaggagacaactgaagctaatgccggtcagtttctctcctttgcaagtctattccttccgaaattggtagtaggagaaaaggcttgccttgagaaggttcaaaggcaaattcaagtacatgcagagcaaggactgatacaatatccaacagcttggcaatcagtaggacacatgatggtgattttccgtttgatgcgaacaaattttctgatcaaatttctcctaatacaccaagggatgcacatggttgccgggcatgatgccaacgatgctgtgatttcaaattcagtggctcaagctcgtttttcaggcttattgattgtcaaaacagtacttgatcatatcctacaaaagacagaacgaggagttcgtctccatcctcttgcaaggaccgccaaggtaaaaaatgaggtgaactcctttaaggctgcactcagctccctggccaagcatggagagtatgctcctttcgcccgacttttgaacctttctggagtaaataatcttgagcatggtcttttccctcaactatcggcaattgcactcggagtcgccacagcacacgggagtaccctcgcaggagtaaatgttggagaacagtatcaacaactcagagaggctgccactgaggctgagaagcaactccaacaatatgcagagtctcgcgaacttgaccatcttggacttgatgatcaggaaaagaaaattcttatgaacttccatcagaaaaagaacgaaatcagcttccagcaaacaaacgctatggtaactctaagaaaagagcgcctggccaagctgacagaagctatcactgctgcgtcactgcccaaaacaagtggacattacgatgatgatgacgacattccctttccaggacccatcaatgatgacgacaatcctggccatcaagatgatgatccgactgactcacaggatacgaccattcccgatgtggtggttgatcccgatgatggaagctacggcgaataccagagttactcggaaaacggcatgaatgcaccagatgacttggtcctattcgatctagacgaggacgacgaggacactaagccagtgcctaatagatcgaccaagggtggacaacagaagaacagtcaaaagggccagcatatagagggcagacagacacaatccaggccaattcaaaatgtcccaggccctcacagaacaatccaccacgccagtgcgccactcacggacaatgacagaagaaatgaaccctccggctcaaccagccctcgcatgctgacaccaattaacgaagaggcagacccactggacgatgccgacgacgagacgtctagccttccgcccttggagtcagatgatgaagagcaggacagggacggaacttccaaccgcacacccactgtcgccccaccggctcccgtatacagagatcactctgaaaagaaagaactcccgcaagacgagcaacaagatcaggaccacactcaagaggccaggaaccaggacagtgacaacacccagtcagaacactcttttgaggagatgtatcgccacattctaagatcacaggggccatttgatgctgttttgtattatcatatgatgaaggatgagcctgtagttttcagtaccagtgatggcaaagagtacacgtatccagactcccttgaagaggaatatccaccatggctcactgaaaaagaggctatgaatgaagagaatagatttgttacattggatggtcaacaattttattggccggtgatgaatcacaagaataaattcatggcaatcctgcaacatcatcagtga

SEQ ID NO: 60 - RNA, 2220 nt, synthetic construct
  misc_feature 1..2220: note = EBOV NP, Sierra Leone 2014, CDS mRNA wild type
  source 1..2220: mol_type = other RNA
atggattctcgtcctcagaaagtctggatgacgccgagtctcactgaatctgacatggattaccacaagatcttgacagcaggtctgtccgttcaacaggggattgttcggcaaagagtcatcccagtgtatcaagtaaacaatcttgaggaaatttgccaacttatcatacaggcctttgaagctggtgttgattttcaagagagtgcggacagtttccttctcatgctttgtcttcatcatgcgtaccaaggagattacaaacttttcttggaaagtggcgcagtcaagtatttggaagggcacgggttccgttttgaagtcaagaagtgtgatggagtgaagcgccttgaggaattgctgccagcagtatctagtgggagaaacattaagagaacacttgctgccatgccggaagaggagacgactgaagctaatgccggtcagttcctctcctttgcaagtctattccttccgaaattggtagtaggagaaaaggcttgccttgagaaggttcaaaggcaaattcaagtacatgcagagcaaggactgatacaatatccaacagcttggcaatcagtaggacacatgatggtgattttccgtttgatgcgaacaaattttttgatcaaatttcttctaatacaccaagggatgcacatggttgccggacatgatgccaacgatgctgtgatttcaaattcagtggctcaagctcgtttttcaggtctattgattgtcaaaacagtacttgatcatatcctacaaaagacagaacgaggagttcgtctccatcctcttgcaaggaccgccaaggtaaaaaatgaggtgaactccttcaaggctgcactcagctccctggccaagcatggagagtatgctcctttcgcccgacttttgaacctttctggagtaaataatcttgagcatggtcttttccctcaactgtcggcaattgcactcggagtcgccacagcccacgggagcaccctcgcaggagtaaatgttggagaacagtatcaacagctcagagaggcagccactgaggctgagaagcaactccaacaatatgcggagtctcgtgaacttgaccatcttggacttgatgatcaggaaaagaaaattcttatgaacttccatcagaaaaagaacgaaatcagcttccagcaaacaaacgcgatggtaactctaagaaaagagcgcctggccaagctgacagaagctatcactgctgcatcactgcccaaaacaagtggacattacgatgatgatgacgacattccctttccaggacccatcaatgatgacgacaatcctggccatcaagatgatgatccgactgactcacaggatacgaccattcccgatgtggtagttgaccccgatgatggaggctacggcgaataccaaagttactcggaaaacggcatgagtgcaccagatgacttggtcctattcgatctagacgaggacgacgaggacaccaagccagtgcctaacagatcgaccaagggtggacaacagaaaaacagtcaaaagggccagcatacagagggcagacagacacaatccacgccaactcaaaacgtcacaggccctcgcagaacaatccaccatgccagtgctccactcacggacaatgacagaagaaacgaaccctccggctcaaccagccctcgcatgctgaccccaatcaacgaagaggcagacccactggacgatgccgacgacgagacgtctagccttccgcccttagagtcagatgatgaagaacaggacagggacggaacttctaaccgcacacccactgtcgccccaccggctcccgtatacagagatcactccgaaaagaaagaactcccgcaagatgaacaacaagatcaggaccacattcaagaggccaggaaccaagacagtgacaacacccagccagaacattcttttgaggagatgtatcgccacattctaagatcacaggggccatttgatgccgttttgtattatcatatgatgaaggatgagcctgtagttttcagtaccagtgatggtaaagagtacacgtatccggactcccttgaagaggaatatccaccatggctcactgaaaaagaggccatgaatgatgagaatagatttgttacactggatggtcaacaattttattggccagtaatgaatcacaggaataaattcatggcaatcctgcaacatcatcagtga

SEQ ID NO: 61 - RNA, 2031 nt, synthetic construct
  misc_feature 1..2031: note = BDBV GP, Uganda 2007, CDS mRNA wild type
  source 1..2031: mol_type = other RNA
atggttacatcaggaattctacaattgccccgtgaacgcttcagaaaaacatcattttttgtttgggtaataatcctatttcacaaagttttccctatcccattgggcgtagttcacaacaacactctccaggtaagtgatatagataaattggtgtgccgggataaactttcctccacaagtcagctgaaatcggtcgggcttaatctagaaggtaatggagttgccacagatgtaccaacagcaacgaagagatggggattccgagctggtgttccacccaaagtggtgaactacgaagctggggagtgggctgaaaactgctacaacctggacatcaagaaagcagatggtagcgaatgcctacctgaagcccctgagggtgtaagaggcttccctcgctgccgttatgtgcacaaggtttctggaacagggccgtgccctgaaggttacgctttccacaaagaaggcgctttcttcctgtatgatcgactggcatcaacaatcatctatcgaagcaccacgttttcagaaggtgttgtggctttcttgatcctccccgaaactaaaaaggactttttccaatcgccaccactacatgaaccggccaatatgacaacagacccatccagctactaccacacagtcacacttaattatgtggctgacaattttgggaccaatatgactaactttctgtttcaagtggatcatctaacttatgtgcaacttgaaccaagattcacaccacaatttcttgtccaactcaatgagaccatttatactaatgggcgtcgcagcaacaccacaggaacactaatttggaaagtaaatcctactgttgacaccggcgtaggtgaatgggccttctgggaaaataaaaaaaacttcacaaaaaccctttcaagtgaagagctgtctgtcatatttgtaccaagagcccaggatccaggcagcaaccagaagacgaaggtcactcccaccagcttcgccaacaaccaaacctccaagaaccacgaagacttggttccagaggatcccgcttcagtggttcaagtgcgagacctccagagggaaaacacagtgccgaccccacccccagacacagtccccacaactctgatccccgacacaatggaggaacaaaccaccagccactacgaaccaccaaacatttccagaaaccatcaagagaggaacaacaccgcacaccccgaaactctcgccaacaatcccccagacaacacaaccccgtcgacaccacctcaagacggtgagcggacaagttcccacacaacaccctccccccgcccagtcccaaccagcacaatccatcccaccacacgagagactcacattcccaccacaatgacaacaagccatgacaccgacagcaatcgacccaacccaattgacatcagcgagtctacagagccaggaccactcaccaacaccacaagaggggctgcaaatctgctgacaggctcaagaagaacccgaagggaaatcaccctgagaacacaagccaaatgcaacccaaacctacactattggacaacccaagatgaaggggctgccattggtttagcctggataccttacttcgggcccgcagcagagggaatttatacggaagggataatgcacaatcaaaatgggctaatttgcgggttgaggcagctagcaaatgagacgactcaagccctacagttattcttgcgtgctaccacggaattgcgcactttctctatattgaatcgaaaagccatcgactttttactccaaagatggggaggaacgtgccacatcttaggcccagattgctgtattgagccccatgattggactaagaacattactgacaaaatagatcaaatcattcatgatttcattgataaacctctaccagatcaaacagataatgacaattggtggacagggtggaggcaatgggttcctgccgggatcgggatcacgggggtaataatcgcagttatagcactgctgtgtatttgcaaatttctactctaa

SEQ ID NO: 62 - RNA, 2031 nt, synthetic construct
  misc_feature 1..2031: note = SUDV GP, Gulu, Uganda 2007, CDS mRNA wild type
  source 1..2031: mol_type = other RNA
atggggggtcttagcctactccaattgcccagggacaaatttcggaaaagctctttctttgtttgggtcatcatcttattccaaaaggccttttccatgcctttgggtgttgtgactaacagcactttagaagtaacagagattgaccagctagtctgcaaggatcatcttgcatctactgaccagctgaaatcagttggtctcaacctcgaggggagcggagtatctactgatatcccatctgcaacaaagcgttggggcttcagatctggtgttcctcccaaggtggtcagctatgaagcgggagaatgggctgaaaattgctacaatcttgaaataaagaagccggacgggagcgaatgcttacccccaccgccagatggtgtcagaggctttccaaggtgccgctatgttcacaaagcccaaggaaccgggccctgcccaggtgactacgcctttcacaaggatggagctttcttcctctatgacaggctggcttcaactgtaatttacagaggagtcaattttgctgagggggtaattgcattcttgatattggctaaaccaaaagaaacgttccttcagtcaccccccattcgagaggcagtaaactacactgaaaatacatcaagttattatgccacatcctacttggagtatgaaatcgaaaattttggtgctcaacactccacgacccttttcaaaattgacaataatacttttgttcgtctggacaggccccacacgcctcagttccttttccagctgaatgataccattcaccttcaccaacagttgagtaatacaactgggagactaatttggacactagatgctaatatcaatgctgatattggtgaatgggctttttgggaaaataaaaaaaatctctccgaacaactacgtggagaagagctgtctttcgaagctttatcgctcaacgagacagaagacgatgatgcggcatcgtcgagaattacaaagggaagaatctccgaccgggccaccaggaagtattcggacctggttccaaagaattcccctgggatggttccattgcacataccagaaggggaaacaacattgccgtctcagaattcgacagaaggtcgaagagtaggtgtgaacactcaggagaccattacagagacagctgcaacaattataggcactaacggcaaccatatgcagatctccaccatcgggataagaccgagctccagccaaatcccgagttcctcaccgaccacggcaccaagccctgaggctcagacccccacaacccacacatcaggtccatcagtgatggccaccgaggaaccaacaacaccaccgggaagctcccccggcccaacaacagaagcacccactctcaccaccccagaaaatataacaacagcggttaaaactgtcctgccacaggagtccacaagcaacggtctaataacttcaacagtaacagggattcttgggagtcttgggcttcgaaaacgcagcagaagacaaactaacaccaaagccacgggtaagtgcaatcccaacttacactactggactgcacaagaacaacataatgctgctgggattgcctggatcccgtactttggaccgggtgcggaaggcatatacactgaaggcctgatgcataaccaaaatgccttagtctgtggacttaggcaacttgcaaatgaaacaactcaagctctgcagcttttcttaagagccacaacggagctgcggacatataccatactcaataggaaggccatagatttccttctgcgacgatggggcgggacatgcaggatcctgggaccagattgttgcattgagccacatgattggacaaaaaacatcactgataaaatcaaccaaatcatccatgatttcatcgacaaccccttacctaatcaggataatgatgataattggtggacgggctggagacagtggatccctgcaggaataggcattactggaattattattgcaattattgctcttctttgcgtttgcaagctgctttgctga

SEQ ID NO: 63 - RNA, 2031 nt, synthetic construct
  misc_feature 1..2031: note = TAFV GP, Cote dIvoire 1994, CDS mRNA wild type
  source 1..2031: mol_type = other RNA
atgggagcgtcagggattctgcaattgccccgtgagcgcttcaggaaaacatctttctttgtttgggtaataatcctattccataaagtcttttcaatcccgttgggggttgtacacaacaataccctacaagtgagtgatattgacaagtttgtgtgccgagacaaactctcttcaactagccaattgaagtcagtcgggttgaacttggagggcaatggagtagcaactgatgtaccaacggcaaccaaaagatggggttttcgagctggtgttccaccaaaggtggtaaattgcgaagctggagaatgggctgagaactgttataacctggctataaagaaagttgatggtagtgagtgcctaccagaagcccctgagggagtgagggattttccccgttgccgctatgtacacaaagtctcaggaactggaccatgcccaggaggactcgcctttcacaaagaaggagccttcttcctgtatgaccgactcgcatcaacaatcatttatcggggtacaacctttgccgaaggagttattgcatttctgatcttgcctaaggcgcgaaaggattttttccagtctcctccattgcatgagcctgccaacatgaccacggatccctccagttactatcacacgacaacaataaactacgtggttgataattttggaaccaacaccacagagtttctgttccaagtcgatcatttgacgtatgtgcagctcgaggcaagattcacaccacaattccttgtcctcctaaatgaaaccatctactctgataaccgcagaagtaacacaacaggaaaactaatctggaaaataaatcccactgttgataccagcatgggtgagtgggctttctgggaaaataaaaaaaacttcacaaaaaccctttcaagtgaagagttgtctttcgtacctgtaccagaaacccagaaccaggtccttgacacgacagcgacggtctctcctcccatctccgcccacaaccacgcagccgaagaccacaaagaattggtttcagaggattccactccagtggttcagatgcaaaacatcaagggaaaggacacaatgccaaccacagtgacgggtgtaccaacaaccacaccctctccatttccaatcaatgctcgcaacactgatcataccaaatcatttatcggcctggaggggccccaagaagaccacagcaccacacagcctgccaagaccaccagccaaccaaccaacagcacagaatcgacgacactaaacccaacatcagagccctccagtagaggcacgggaccatccagccccacggtccccaacaccacagaaagccacgccgaacttggcaagacaaccccaaccacactcccagaacagcacactgccgccagtgccattccaagagccgtgcaccccgacgaactcagtggacctggcttcctgacgaacacaatacggggggttacaaatctcctgacaggatccagaagaaagcgaagggatgtcactcccaatacacaacccaaatgcaacccaaacctgcactattggacagccttggatgagggtgctgccataggtttagcctggataccatacttcgggccagcagctgagggaatttacactgaaggcataatggagaatcaaaatggattgatctgtggattgaggcagctggccaacgaaacgacacaagctcttcaattgttcttaagggcaactactgagttgcgtacattctctatactaaatcggaaagcaatagacttcttgctccaaagatggggaggaacatgtcacattctagggcctgattgttgcattgaaccccaagattggaccaaaaatatcactgataaaattgatcaaataatccatgactttgtcgataataatcttccaaatcagaatgatggcagcaactggtggactggatggaaacaatgggttcctgctggaataggaatcacaggagtaatcattgctattattgctttgctgtgcatttgcaaattcatgctttga

SEQ ID NO: 64 - RNA, 981 nt, synthetic construct
  misc_feature 1..981: note = BDBV VP40, Uganda 2007, CDS mRNA wild type
  source 1..981: mol_type = other RNA
atgaggagggcaattctacctactgcaccgccagaatacatagaggctgtctacccaatgagaacggttagtactagtatcaacagtactgccagtggtccgaactttccagcaccggatgtaatgatgagtgatacaccctccaactcactccgaccaattgctgatgataacatcgatcatccaagtcatacaccaaccagtgtttcatcagcctttatactcgaggcaatggtgaatgtgatatcggggccgaaggtactaatgaagcaaattcctatatggctccccttgggtgttgctgatcaaaaaacatatagttttgactcaactacagctgcaattatgctcgcatcgtacaccatcactcactttggcaaaacctccaatccgcttgtgagaatcaatcgacttggtcctgggatccccgatcacccgttgcggcttctaagaataggaaatcaagccttcttgcaagagtttgtgctgcctccagttcaattgccgcagtatttcacttttgacctgacggctctaaagctgatcactcaacctctcccggcagcaacctggacggatgatactccgaccggtcctacaggaatacttcgtcctggaatttcctttcatcccaaactgagacctatcctattgccagggaagaccgggaaaagaggatccagctccgatcttacttctcctgataaaatacaagcaataatgaactttctccaagacctcaaactcgtgccgattgatccagccaagaacattatgggtattgaagtgccggaactcttggtccacagactaactggaaagaaaatcacaacaaaaaatggtcaaccaataattcctattcttctaccaaagtatattggcatggatcccatttctcagggagacctcacaatggtcatcactcaagactgtgacacttgccattctcctgctagtcttcctccagtcagcgagaaatga

SEQ ID NO: 65 - RNA, 981 nt, synthetic construct
  misc_feature 1..981: note = SUDV VP40, Gulu, Uganda 2000, CDS mRNA wild type
  source 1..981: mol_type = other RNA
atgagaagggtcactgtgccgactgcaccacctgcctatgctgacattggctatcctatgagcatgcttcccatcaagtcaagcagggctgtgagtggaattcaacagaaacaagaggtccttcctggaatggatacaccatcaaattctatgagacctgttgctgatgataacattgatcatacaagtcataccccgaacggagtggcctcagcattcatcttggaggcaactgtcaatgtgatctcggggcccaaagtcctcatgaaacaaatccctatttggttgccactcggaattgctgaccaaaaaacgtacagttttgactcaacaacagcagcaattatgctcgcatcttatacgatcacccattttggaaaggccaacaaccccctcgttagagtgaatcgacttggtcagggaataccggatcacccactcagattgctcaggatggggaaccaggctttccttcaagagtttgtgctaccaccagttcaactgccgcaatatttcacttttgatctgactgcactcaaactagtgacacagcctctccctgctgcaacatggacagatgagactccgagcaacctttcaggagcccttcgtcccgggctttcatttcacccaaagctgagacccgttctacttccaggcaagacgggaaagaaagggcatgtttctgatctgactgccccagacaaaattcagacaattgtgaacctgatgcaagatttcaaaatcgtgccaattgatccagctaagagtatcattgggatcgaggttccagaattgctggtccacaagctcactgggaagaaaatgagtcagaagaatggacagcctataattcctgtcttactcccaaaatacattgggctagatccaatctcacctggagacctgactatggtcataacaccagattatgatgattgtcattcacctgccagttgctcttatctcagtgaaaagtga

SEQ ID NO: 66 - RNA, 981 nt, synthetic construct
  misc_feature 1..981: note = TAFV VP40, Cote dIvoire 1994, CDS mRNA wild type
  source 1..981: mol_type = other RNA
atgaggagaatcatcctacccacggcaccacctgaatacatggaggctgtttacccaatgagaacaatgaattctggtgcagacaacactgccagtggccctaattacacaacaactggtgtgatgacaaatgatactccctctaattcactccgaccagttgcagatgataatattgatcatccgagccacacgcctaacagtgttgcctctgcatttatattggaagctatggtgaatgtaatatctggcccgaaagtgctgatgaagcaaatcccaatctggcttcctctgggtgtctctgaccagaagacatatagctttgattcaaccactgctgccattatgctagcatcatataccatcactcattttggcaaaacctcaaatccccttgtgagaatcaaccgacttggtcctggcatacctgatcacccactacgactcctaagaataggaaatcaagccttcctacaagagtttgtgctacctcctgtacaactgccacaatacttcacttttgatctgacagcgctgaagctgatcacccagccactcccagcggcaacctggacagatgaaactccagctgtgtcaactggcacgctccgcccagggatctcattccatcccaaattaaggcctatcctgctaccaggaagagctggaaagaagggctccaactccgatctaacatctcctgacaaaatccaggctataatgaatttcctacaagacctcaaaattgtaccaatcgatccaaccaagaatatcatgggtattgaagtgccagaactcctggttcacaggctgactgggaagaagacaactaccaagaatggtcaaccaatcattccaattctgctaccaaagtacattggtcttgatcctctatctcaaggtgatctcacaatggtgatcactcaggactgtgattcctgccactccccggccagtcttcccccagtcaatgaaaaatga

SEQ ID NO: 67 - RNA, 2088 nt, synthetic construct
  misc_feature 1..2088: note = MARV NP, Angola 2005, CDS mRNA wild type
  source 1..2088: mol_type = other RNA
atggatttacatagtttgttagaattgggtacaaaacctactgctcctcatgttcgtaataagaaggtgatattattcgacacaaatcatcaggttagtatctgtaatcagataatagatgcaataaactcagggattgatcttggagatcttctagaagggggtttgctgacgttgtgtgttgaacactactataattccgacaaagataaattcaacacaagtcctatcgcaaaatacttgcgtgatgcgggttatgaatttgatgtcatcaagaatgcagatgcaacccgctttctggatgtgatccccaatgaacctcattacagccctttaattctggcccttaaaacattagaaagcactgaatctcagagggggaggattgggctctttttgtcattctgcagtctttttctcccaaaacttgtcgtcggagaccgggccagtatcgaaaaggccttaagacaagtgacagtacatcaagaacagggaattgtcacataccctaatcattggcttactacaggtcacatgaaagtaatttttgggatcttgaggtctagctttattttaaagtttgtgttgattcatcaaggagtaaatttggtgacaggtcatgatgcatatgacagtatcattagcaattcagtaggtcaaactagattctcaggacttcttattgtgaaaacagttctcgagttcatcttgcaaaaaactgattcaggggtgacactacatcctttggtgcggacctccaaagtgaaaaacgaagttgctagtttcaagcaggcgttgagcaacttagcccgacacggagagtacgcaccatttgcacgggttttgaatttatcagggattaacaatctcgaacatggactctatcctcagctctcagcaattgcgctgggtgtagcgacagcacatggcagtacattggctggtgtcaatgtcggcgaacaatatcaacagctacgagaggcagcacatgatgcagaagtaaaactacaaagacgacatgaacatcaggaaattcaagctattgccgaagatgacgaggaaaggaagatattagaacaattccaccttcagaaaactgaaatcacacacagtcagacactcgccgtcctcagccagaaacgagaaaaattagcccgccttgctgcagaaattgaaaacaatattgtggaagatcagggatttaaacaatcacagaatcgggtgtcacagtcgtttttgaatgaccccacacctgtggaagtaacggttcaagccaggcccataaatcgaccaactgctctgcctcctccagtcgacagcaaaatcgagcatgaatctacagaagatagctcttcttcaagtagctttgttgacttgaatgatccatttgcactgctgaatgaggacgaagacactcttgatgatagtgtcatgatcccgagcacaacatcgagagaatttcaagggattcctgaaccgccaagacaatctcaggacatcgataacagccaaggaaagcaagaagatgaatcaacaaacctgattaagaaaccgtttttgcgatatcaagaactgcctcctgtccaggaggatgatgaatcggaatacacaactgactctcaggaaagtattgaccagccaggatccgacaatgaacaaggggttgatcttccacctcctccgttgtacgctcaggaaaagagacaggacccaatacagcacccagcagtaagctctcaggatccctttggcagtattggtgatgtaaatggtgatatcttagaacccataaggtcaccctcttcaccgtctgctcctcaggaagacacaagggcaagagaagcctatgaattgtcacctgatttcacaaattatgaggataatcaacagaattggccacaaagagtggtgacaaagaagggtaggactttcctttatcctaatgatcttctgcagacaaatcctccagaatcacttataacagcccttgttgaggaataccaaaatcctgtctcagccaaagagcttcaagcagattggcccgacatgtcatttgatgaaaggagacatgttgctatgaacttgtaa

SEQ ID NO: 68 - RNA, 2220 nt, synthetic construct
  misc_feature 1..2220: note = BDBV NP, Uganda 2007, CDS mRNA wild type
  source 1..2220: mol_type = other RNA
atggatcctcgtccaatcagaacctggatgatgcataacacatctgaagttgaagcagactaccataagattctaactgccggattgtccgtccagcaaggcattgtgagacaaagaatcattcctgtttaccaaatctcaaacctggaggaagtatgtcaactcatcatacaggcattcgaggctggcgtcgacttccaggatagtgcagatagctttttgttaatgctatgtctgcatcatgcctatcaaggggattataaacaatttttggaaagtaatgcggtaaaataccttgaaggtcatggattccgttttgagatgaagaaaaaggaaggtgtcaagcgcctggaggaactactccctgctgcctcgagtggaaagaacatcaagagaacattggctgcaatgcccgaggaggaaacaacagaagcaaatgctggacaatttctttcatttgctagtctgtttctcccaaaattggttgtcggagaaaaggcctgtctggagaaggttcaacgacaaatccaagtgcacgcagaacaaggtctgattcaatacccgacatcttggcaatcggtgggacatatgatggtcatcttcagactaatgcgaaccaacttcctgattaagttcctcctaatacatcaaggaatgcatatggttgcagggcatgatgctaatgatgccgtcattgccaactctgtagctcaagctcgtttctccggattgttgatagtcaaaacagtgcttgatcatatcctccaaaaaacagagcacggagttcgcctgcatcccttggcgcgaacagccaaagtcaaaaatgaggtgagctcttttaaggccgctttagcctcactagcacaacatggagaatatgccccgtttgctcgtctgctgaatctatctggggttaataatcttgagcatgggcttttccctcaactttctgcaattgctttgggagtagcaactgcacatgggagcactctggctggagtcaatgtaggagagcaataccaacaactgcgagaagcagccactgaggccgaaaagcagttgcagaaatatgctgaatctcgtgaacttgatcacctaggtcttgatgatcaggaaaagaaaatcctaaaagacttccatcagaaaaagaatgagatcagcttccagcagacgacagccatggtcacactgcggaaagagagattggccaaattgaccgaagctattacttccacctctatcctcaaaacaggaaggcggtatgatgatgacaatgacataccctttccagggccaatcaatgataacgagaactctggtcagaacgatgacgatccaacagactcccaggataccacaatcccggatgtaataatcgatccaaacgatggtgggtataataattacagcgattatgcaaatgatgctgcaagtgctcctgatgacctagttctttttgaccttgaggacgaggatgatgctgataacccggctcaaaacacgccagaaaaaaatgatagaccagcaacaacaaagctgagaaatggacaggaccaggatggaaaccaaggcgaaactgcatccccacgggtagcccccaaccaatacagagacaagccaatgccacaagtacaggacagatccgaaaatcatgaccaaacccttcaaacacagtccagggttttgactcctatcagcgaggaagcagaccccagcgaccacaacgatggtgacaatgaaagcattcctcccctggaatcagacgacgagggtagcactgatactactgcagcagaaacaaagcctgccactgcacctcccgctcccgtctaccgaagtatctccgtagatgattctgtcccctcagagaacattcccgcacagtccaatcaaacgaacaatgaggacaatgtcaggaacaatgctcagtcggagcaatccattgcagaaatgtatcaacatatcttgaaaacacaaggaccttttgatgccatcctttactaccatatgatgaaagaagagcccatcattttcagcactagtgatgggaaggagtatacatatccagactctcttgaagatgagtatccaccctggctcagcgagaaggaagccatgaacgaagacaatagattcataaccatggatggtcagcagttttactggcctgtgatgaatcatagaaataaattcatggcaatcctccagcatcacaggtga

SEQ ID NO: 69 - RNA, 2217 nt, synthetic construct
  misc_feature 1..2217: note = SUDV NP, Gulu, Uganda 2000, CDS mRNA wild type
  source 1..2217: mol_type = other RNA
atggataaacgggtgagaggttcatgggccctgggaggacaatctgaagttgatcttgactaccacaaaatattaacagccgggctttcggtccaacaagggattgtgcgacaaagagtcatcccggtatatgttgtgagtgatcttgagggtatttgtcaacatatcattcaggcctttgaagcaggcgtagatttccaagataatgctgacagcttccttttacttttatgtttacatcatgcttaccaaggagatcataggctcttcctcaaaagtgatgcagttcaatacttagagggccatggtttcaggtttgaggtccgagaaaaggagaatgtgcaccgtctggatgaattgttgcccaatgtcaccggtggaaaaaatcttaggagaacattggctgcaatgcctgaagaggagacaacagaagctaatgctggtcagtttttatcctttgccagtttgtttctacccaaacttgtcgttggggagaaagcgtgtctggaaaaagtacaaaggcagattcaggtccatgcagaacaagggctcattcaatatccaacttcctggcaatcagttggacacatgatggtgatcttccgtttgatgagaacaaactttttaatcaagttcctactaatacatcaggggatgcacatggtcgcaggccatgatgcgaatgacacagtaatatctaattctgttgcccaagcaaggttctctggtcttctgattgtaaagactgttctggaccacatcctacaaaaaacagatcttggagtacgacttcatccactggccaggacagcaaaagtcaagaatgaggtcagttcattcaaggcagctcttggctcacttgccaagcatggagaatatgctccatttgcacgtctcctcaatctttctggagtcaacaacttggaacatgggctttatccacaactctcagccattgctttgggtgttgcaactgcccacgggagcacgctggctggtgttaatgtaggggagcaatatcagcaactgcgtgaggctgctactgaagctgaaaagcaactccaacaatatgctgaaacacgtgagttggacaaccttgggcttgatgaacaggaaaagaagattctcatgagcttccaccagaagaagaatgagatcagcttccagcaaactaacgcaatggtaaccctgaggaaagagcggctggccaaactgaccgaagccatcacgactgcatcaaagatcaaggttggagatcgttatcctgatgacaatgatattccatttcccgggccgatctatgatgaaacccaccccaacccttctgatgataatcctgatgattcacgtgatacaactatcccaggtggtgttgttgacccgtatgatgatgagagtaataattatcctgactacgaggattcggctgaaggcaccacaggagatcttgatctcttcaatttggacgacgacgatgacgacagccaaccaggaccaccagacagggggcagagcaaggaaagagcggctcggacacatggcctccaagatccgaccttggacggagcgaaaaaggtgccggagttgaccccaggttcccaccaaccaggcaacctccacatcaccaagccgggttcaaacaccaaccaaccacaaggcaatatgtcatctactctccagagtatgacccctatacaggaagaatcagagcccgatgatcagaaagatgatgatgacgagagtctcacatcccttgactctgaaggtgacgaagatgttgagagcgtatcaggggagaacaacccaactgtagctccaccagcaccagtctacaaagatactggagtagacactaatcagcaaaatggaccaagcaatgctgtagatggtcaaggttctgaaagtgaagctctcccaatcaaccccgaaaagggatctgcactggaagaaacatattatcatctcctaaaaacacagggtccatttgaggcaatcaattattatcacctaatgagtgatgagcccattgcttttagcactgaaagtggcaaggaatatatcttcccagattctcttgaagaagcctacccgccttggttgagtgagaaggaggccttagagaaagaaaatcgttatctggtcattgatggccagcaattcctctggccagtaatgagcctacaggacaagttccttgctgttcttcaacatgactga

SEQ ID NO: 70 - RNA, 2220 nt, synthetic construct
  misc_feature 1..2220: note = TAFV NP, Cote dIvoire 1994, CDS mRNA wild type
  source 1..2220: mol_type = other RNA
atggagagtcgggcccacaaagcatggatgacgcacaccgcatcaggtttcgaaacagattaccataagattttaacagcaggattgtcagtccaacaaggcattgtgagacaacgggtcattcaagtccaccaggttacaaacctagaagaaatatgccaattgatcattcaagcctttgaagctggtgttgattttcaagagagtgcagacagtttcttgctgatgctatgtttacatcatgcttatcagggtgactacaagcaattcttggaaagcaatgcagtcaagtaccttgagggtcatggctttcgctttgaggtcaggaaaaaggaaggagtcaagcgactcgaagaattgcttcctgctgcatccagtggcaagagcatcaggagaacactggctgcaatgcctgaagaggagacaacagaagcaaatgccggacagttcctctcttttgctagcttatttcttcctaagctagttgtcggagaaaaagcctgtctagaaaaggtgcagcggcaaattcaagttcattctgagcagggattgatccaataccccacagcctggcagtcagttggacacatgatggtcattttcagactgatgagaacaaattttctaattaagttcctccttatacatcaagggatgcatatggtagcaggacacgatgctaacgatgctgtcatcgcaaactctgtagctcaagcacgtttttcaggattattgatcgttaaaacagtgctagatcacatccttcagaaaacagagcacggagtgcgtcttcatcctttggcaagaactgctaaggtcaagaacgaagtaaattcctttaaggctgcccttagctcgctagcacaacatggagagtatgctccttttgctcgcttgctgaatctttctggagtcaacaatctcgagcacggactgtttcctcagctttctgcaattgccctaggtgtcgcaacggcacacggcagtaccctggcaggagtaaatgtgggggaacagtatcagcaactacgagaagcagccactgaggcagaaaaacaattgcagaaatacgctgaatctcgcgagcttgaccatctaggtctcgatgatcaagagaagaagatcttgaaagacttccatcagaagaaaaatgaaatcagcttccagcagacaacagccatggtcacactacggaaggaaaggctagccaagctcactgaggcaatcacctccacatcccttctcaagacaggaaaacagtatgatgatgacaacgatatcccctttcctgggcccatcaatgataacgaaaactcagaacagcaagacgatgatccaacagattctcaggacactaccatccctgatatcattgttgacccggatgatggcagatacaacaattatggagactatcctagtgagacggcgaatgcccctgaagaccttgttctttttgaccttgaagatggtgacgaggatgatcaccgaccgtcaagttcatcagagaacaacaacaaacacagtcttacaggaactgacagtaacaaaacaagtaactggaatcgaaacccgactaatatgccaaagaaagactccacacaaaacaatgacaatcctgcacagcgggctcaagaatacgccagggataacatccaggatacaccaacaccccatcgagctctaactcccatcagcgaagaaaccggctccaatggtcacaatgaagatgacattgatagcatccctcctttggaatcagacgaagaaaacaacactgagacaaccattaccaccacaaaaaataccactgctccaccagcacctgtttatcggagtaattcagaaaaggagcccctcccgcaagaaaaatcccagaagcaaccaaaccaagtgagtggtagtgagaataccgacaataaacctcactcagagcaatcagtggaagaaatgtatcgacacatcctccaaacacaaggaccatttgatgccatcctatactattacatgatgacggaggagccgattgtctttagcactagtgatgggaaagaatacgtataccctgattctcttgaaggggagcatccaccgtggctcagtgaaaaagaggccttgaatgaggacaataggtttatcacaatggatgatcaacaattctactggcctgtaatgaatcacaggaacaaattcatggctatccttcagcaccacaagtaa

SEQ ID NO: 71 - RNA, 2031 nt, synthetic construct
  misc_feature 1..2031: note = EBOV GP, Mayinga, Zaire 1976, optimized mRNA Sequence
  source 1..2031: mol_type = other RNA
atgggcgtgaccgggatcctgcagctcccccgcgaccggttcaagcgcaccagcttcttcctgtgggtcatcatcctgttccagcggacgttctccatcccgctcggcgtgatccacaacagcaccctgcaggtgtccgacgtcgacaagctggtgtgccgcgacaagctcagctccaccaaccagctgcggagcgtggggctgaacctcgagggcaacggggtcgccaccgacgtgccctccgccacgaagcgctggggcttccggagcggcgtgccgcccaaggtcgtgaactacgaggcgggggagtgggccgagaactgctacaacctggagatcaagaagcccgacggctccgagtgcctgcccgccgcccccgacgggatccgcggcttcccccggtgccgctacgtgcacaaggtcagcgggaccggcccgtgcgccggcgacttcgcgttccacaaggagggggccttcttcctctacgaccggctggcctccaccgtgatctaccgcggcaccacgttcgccgagggggtggtcgcgttcctgatcctcccccaggccaagaaggacttcttcagctcccaccccctgcgggagcccgtgaacgccaccgaggacccgagctccggctactacagcaccaccatccgctaccaggccacgggcttcgggaccaacgagaccgagtacctgttcgaggtggacaacctcacctacgtccagctggagtcccggttcacgccccagttcctgctccagctgaacgagaccatctacaccagcggcaagcgctccaacaccacggggaagctgatctggaaggtgaaccccgagatcgacaccaccatcggcgagtgggccttctgggagaccaagaagaacctcacgcggaagatccgcagcgaggagctgagcttcaccgtggtctccaacggggcgaagaacatcagcggccagtcccccgcccggaccagctccgacccgggcaccaacacgaccaccgaggaccacaagatcatggccagcgagaactccagcgccatggtgcaggtgcactcccaggggcgcgaggccgcggtcagccacctgaccacgctcgccaccatctccaccagcccccagtccctgaccacgaagcccggccccgacaacagcacccacaacaccccggtgtacaagctggacatctccgaggccacccaggtcgagcagcaccaccggcgcaccgacaacgacagcacggcctccgacacccccagcgccaccaccgcggccgggccgcccaaggccgagaacacgaacacctccaagagcaccgacttcctcgaccccgccaccacgaccagcccccagaaccactccgagaccgccggcaacaacaacacccaccaccaggacacgggggaggagagcgcgtccagcggcaagctgggcctgatcaccaacaccatcgccggggtggccggcctcatcaccgggggccgccggacgcgccgggaggccatcgtgaacgcgcagcccaagtgcaaccccaacctgcactactggaccacccaggacgagggggccgccatcggcctggcctggatcccgtacttcggccccgccgcggaggggatctacatcgagggcctcatgcacaaccaggacgggctgatctgcggcctgcgccagctcgccaacgagaccacgcaggccctgcagctgttcctccgggccaccaccgagctgcgcaccttctccatcctgaaccggaaggccatcgacttcctcctgcagcgctggggcgggacgtgccacatcctgggccccgactgctgcatcgagccgcacgactggaccaagaacatcaccgacaagatcgaccagatcatccacgacttcgtcgacaagaccctgcccgaccagggggacaacgacaactggtggacgggctggcggcagtggatccccgcggggatcggcgtgaccggcgtgatcatcgccgtcatcgccctcttctgcatctgcaagttcgtgttctga

SEQ ID NO: 72 - RNA, 2031 nt, synthetic construct
  misc_feature 1..2031: note = EBOV GP, Sierra Leone 2014, optimized mRNA Sequence
  source 1..2031: mol_type = other RNA
atgggcgtgaccgggatcctgcagctcccccgcgaccggttcaagcgcaccagcttcttcctgtgggtcatcatcctgttccagcggacgttctccatcccgctcggcgtgatccacaacagcaccctgcaggtgtccgacgtcgacaagctggtgtgccgcgacaagctcagctccaccaaccagctgcggagcgtggggctgaacctcgagggcaacggggtcgccaccgacgtgccctccgtgacgaagcgctggggcttccggagcggcgtcccgcccaaggtggtgaactacgaggccggggagtgggcggagaactgctacaacctggagatcaagaagcccgacggctccgagtgcctgcccgccgcccccgacgggatccgcggcttcccccggtgccgctacgtccacaaggtgagcgggaccggcccgtgcgccggcgacttcgccttccacaaggagggggcgttcttcctctacgaccggctggcctccaccgtgatctaccgcggcaccacgttcgccgagggggtcgtggccttcctgatcctcccccaggcgaagaaggacttcttcagctcccaccccctgcgggagcccgtgaacgccaccgaggacccgagctccggctactacagcaccaccatccgctaccaggccacgggcttcgggaccaacgagaccgagtacctgttcgaggtcgacaacctcacctacgtgcagctggagtcccggttcacgccccagttcctgctccagctgaacgagaccatctacgccagcggcaagcgctccaacaccaccgggaagctgatctggaaggtgaaccccgagatcgacacgaccatcggcgagtgggccttctgggagaccaagaagaacctcacccggaagatccgcagcgaggagctgagcttcacggcggtctccaacgggcccaagaacatcagcggccagtccccggcccggaccagctccgaccccgagaccaacaccacgaacgaggaccacaagatcatggccagcgagaactccagcgccatggtgcaggtgcactcccagggccgcaaggccgcggtcagccacctgaccaccctcgccaccatctccacgagcccccagcccccgaccaccaagaccgggcccgacaactccacgcacaacacccccgtgtacaagctggacatcagcgaggccacccaggtcggccagcaccaccggcgcgccgacaacgactccaccgccagcgacaccccgccggcgacgaccgccgccgggcccctgaaggccgagaacaccaacacctccaagagcgccgactccctcgacctggcgacgaccaccagcccccagaactacagcgagaccgccggcaacaacaacacgcaccaccaggacaccggggaggagtccgccagctccggcaagctgggcctcatcaccaacaccatcgccggggtggcgggcctgatcacgggcgggcgccggacccgccgggaggtgatcgtcaacgcccagcccaagtgcaacccgaacctgcactactggaccacccaggacgagggggccgccatcggcctcgcctggatcccctacttcggccccgcggccgaggggatctacacggagggcctgatgcacaaccaggacgggctgatctgcggcctccgccagctggccaacgagaccacccaggccctgcagctcttcctgcgggccaccacggagctgcgcaccttcagcatcctcaaccggaaggcgatcgacttcctgctgcagcgctggggcgggacctgccacatcctgggcccggactgctgcatcgagccccacgactggaccaagaacatcacggacaagatcgaccagatcatccacgacttcgtggacaagaccctccccgaccagggggacaacgacaactggtggaccggctggcggcagtggatccccgccgggatcggcgtgaccggcgtcatcatcgccgtgatcgccctgttctgcatctgcaagttcgtgttctga

SEQ ID NO: 73 - RNA, 2046 nt, synthetic construct
  misc_feature 1..2046: note = MARV GP, Angola 2005, optimized mRNA Sequence
  source 1..2046: mol_type = other RNA
atgaagaccacctgcctgctcatcagcctgatcctgatccagggcgtgaagacgctccccatcctggagatcgcctccaacatccagccccagaacgtcgacagcgtgtgctccgggaccctgcagaagaccgaggacgtgcacctcatgggcttcaccctgagcgggcagaaggtcgccgactccccgctggaggcgagcaagcgctgggccttccgggccggcgtgccgcccaagaacgtggagtacacggagggggaggaggccaagacctgctacaacatctccgtcaccgaccccagcggcaagtccctcctgctggacccgcccaccaacatccgcgactaccccaagtgcaagacgatccaccacatccagggccagaacccgcacgcccaggggatcgcgctccacctgtggggcgccttcttcctgtacgaccggatcgccagcaccaccatgtaccgcgggaaggtgttcaccgagggcaacatcgccgcgatgatcgtgaacaagacggtccacaagatgatcttctcccggcaggggcagggctaccgccacatgaacctcaccagcaccaacaagtactggacctccagcaacggcacgcagaccaacgacaccgggtgcttcggcaccctgcaggagtacaactccacgaagaaccagacctgcgcccccagcaagaagcccctgcccctcccgaccgcccaccccgaggtgaagctgacctccacgagcaccgacgccaccaagctgaacaccacggaccccaactccgacgacgaggacctcaccaccagcgggagcggctccggcgagcaggagccctacaccacgagcgacgccgcgaccaagcaggggctgtccagcaccatgccgcccaccccgtccccgcagcccagcacgccccagcagggcgggaacaacaccaaccactcccagggcgtggtcaccgagcccgggaagaccaacaccacggcccagcccagcatgccgccccacaacaccaccaccatctccacgaacaacaccagcaagcacaacctgtccacccccagcgtgcccatccagaacgccaccaactacaacacgcagtccaccgccccggagaacgagcagaccagcgccccctccaagaccacgctcctgcccaccgagaacccgaccaccgcgaagagcacgaactccaccaagagccccaccaccacggtgcccaacaccaccaacaagtactccaccagccccagcccgacgcccaactccaccgcccagcacctggtctacttccggcgcaagcggaacatcctctggcgcgagggcgacatgttccccttcctggacggcctgatcaacgcccccatcgacttcgacccggtgcccaacaccaagaccatcttcgacgagagctccagctccggggccagcgccgaggaggaccagcacgcgtcccccaacatcagcctcacgctgtcctacttccccaaggtgaacgagaacaccgcccacagcggcgagaacgagaacgactgcgacgccgagctgcggatctggtccgtccaggaggacgacctcgccgccgggctgagctggatcccgttcttcggccccgggatcgagggcctgtacaccgcgggcctcatcaagaaccagaacaacctggtgtgccgcctgcggcgcctcgccaaccagaccgccaagtccctggagctgctcctgcgggtgacgaccgaggagcgcaccttcagcctgatcaaccggcacgccatcgacttcctcctggcgcgctggggcgggacctgcaaggtcctggggcccgactgctgcatcggcatcgaggacctgtcccggaacatcagcgagcagatcgaccagatcaagaaggacgagcagaaggaggggacgggctggggcctcgggggcaagtggtggacctccgactggggcgtgctgaccaacctggggatcctcctgctgctcagcatcgccgtgctgatcgccctgagctgcatctgccgcatcttcaccaagtacatcggctga

SEQ ID NO: 74 - RNA, 981 nt, synthetic construct
  misc_feature 1..981: note = EBOV VP40, Mayinga, Zaire 1976, optimized mRNA Sequence
  source 1..981: mol_type = other RNA
atgcgccgggtgatcctgcccaccgccccgcccgagtacatggaggccatctaccccgtccgcagcaactccaccatcgcgcggggcgggaacagcaacacgggcttcctcacccccgagtccgtgaacggggacaccccgagcaaccccctgcgccccatcgccgacgacaccatcgaccacgcctcccacacgcccggcagcgtgtccagcgccttcatcctggaggccatggtcaacgtgatctccgggccgaaggtgctcatgaagcagatccccatctggctgcccctgggcgtcgcggaccagaagacctacagcttcgactccaccaccgccgccatcatgctcgccagctacacgatcacccacttcggcaaggcgaccaaccccctggtgcgggtgaaccgcctggggccgggcatccccgaccaccccctccggctgctgcgcatcgggaaccaggccttcctccaggagttcgtcctgcccccggtgcagctgccccagtacttcaccttcgacctcacggccctgaagctgatcacccagcccctccccgccgccacctggaccgacgacacgccgaccggctccaacggggcgctgcggcccggcatcagcttccaccccaagctgcgccccatcctcctgccgaacaagtccggcaagaaggggaacagcgccgacctgacctcccccgagaagatccaggccatcatgaccagcctccaggacttcaagatcgtgcccatcgaccccacgaagaacatcatgggcatcgaggtcccggagaccctggtgcacaagctgaccgggaagaaggtgacctccaagaacggccagcccatcatccccgtcctcctgccgaagtacatcggcctggaccccgtggcccccggggacctcacgatggtgatcacccaggactgcgacacctgccacagccccgccagcctgccggcggtcatcgagaagtga

SEQ ID NO: 75 - RNA, 981 nt, synthetic construct
  misc_feature 1..981: note = EBOV VP40, Sierra Leone 2014,optimized mRNA Sequence
  source 1..981: mol_type = other RNA
atgcgccgggtgatcctgcccaccgccccgcccgagtacatggaggccatctaccccgcgcgcagcaactccaccatcgcccggggcgggaacagcaacacgggcttcctcacccccgagtccgtcaacggggacaccccgagcaaccccctgcgccccatcgccgacgacaccatcgaccacgcctcccacacgcccggcagcgtgtccagcgccttcatcctggaggcgatggtgaacgtcatctccgggccgaaggtgctcatgaagcagatccccatctggctgcccctgggcgtggccgaccagaagacctacagcttcgactccaccaccgccgccatcatgctcgcgagctacacgatcacccacttcggcaaggccaccaaccccctggtccgggtgaaccgcctggggccgggcatccccgaccaccccctccggctgctgcgcatcgggaaccaggccttcctccaggagttcgtgctgcccccggtccagctgccccagtacttcaccttcgacctcacggccctgaagctgatcacccagcccctccccgccgcgacctggaccgacgacacgccgaccggctccaacggggccctgcggcccggcatcagcttccaccccaagctgcgccccatcctcctgccgaacaagtccggcaagaaggggaacagcgccgacctgacctcccccgagaagatccaggccatcatgaccagcctccaggacttcaagatcgtgcccatcgaccccacgaagaacatcatgggcatcgaggtgccggagaccctggtccacaagctgaccgggaagaaggtgacctccaagaacggccagcccatcatccccgtgctcctgccgaagtacatcggcctggaccccgtcgcccccggggacctcacgatggtgatcacccaggactgcgacacctgccacagccccgcgagcctgccggccgtggtcgagaagtga

SEQ ID NO: 76 - RNA, 912 nt, synthetic construct
  misc_feature 1..912: note = MARV VP40, Angola 2005,optimized mRNA Sequence
  source 1..912: mol_type = other RNA
atggccagctccagcaactacaacacctacatgcagtacctgaacccgccgccctacgccgaccacggcgcgaaccagctcatccccgccgaccagctgtccaaccagcaggggatcacccccaactacgtgggcgacctgaacctcgacgaccagttcaaggggaacgtctgccacgccttcacgctggaggccatcatcgacatcagcgcctacaacgagcgcaccgtgaagggcgtgccggcgtggctgcccctcgggatcatgtccaacttcgagtaccccctggcccacaccgtcgccgccctgctcaccggcagctacacgatcacccagttcacccacaacggccagaagttcgtgcgggtgaaccgcctggggaccggcatccccgcgcacccgctgcggatgctccgcgaggggaaccaggccttcatccagaacatggtcatcccccggaacttctccacgaaccagttcacctacaacctgaccaacctggtgctcagcgtgcagaagctgcccgacgacgcctggcgcccctccaaggacaagctgatcggcaacaccatgcaccccgccgtcagcgtgcaccccaacctcccgcccatcgtgctgccgacggtcaagaagcaggcctaccggcagcacaagaaccccaacaacgggcccctgctcgcgatctccggcatcctgcaccagctgcgcgtggagaaggtgcccgagaagaccagcctcttccggatctccctgccggccgacatgttcagcgtcaaggagggcatgatgaagaagcgcggggagaactcccccgtggtgtacttccaggcccccgagaacttccccctgaacggcttcaacaaccggcaggtcgtgctcgcctacgccaacccgaccctgagcgcggtgtga

SEQ ID NO: 77 - RNA, 2220 nt, synthetic construct
  misc_feature 1..2220: note = EBOV NP, Zaire 1976, optimized mRNA Sequence
  source 1..2220: mol_type = other RNA
atggacagccgcccccagaagatctggatggccccgtccctgaccgagagcgacatggactaccacaagatcctcaccgccggcctgtccgtgcagcaggggatcgtccggcagcgcgtgatccccgtgtaccaggtcaacaacctggaggagatctgccagctcatcatccaggcgttcgaggccggcgtggacttccaggagagcgccgactccttcctgctgatgctctgcctgcaccacgcctaccagggggactacaagctgttcctcgagagcggcgccgtgaagtacctggaggggcacggcttccggttcgaggtcaagaagcgcgacggcgtgaagcggctggaggagctcctgcccgcggtgtccagcgggaagaacatcaagcgcacgctggccgccatgcccgaggaggagaccaccgaggccaacgcgggccagttcctctccttcgccagcctgttcctgccgaagctcgtcgtgggggagaaggcctgcctggagaaggtgcagcggcagatccaggtccacgccgagcagggcctgatccagtaccccaccgcctggcagtccgtggggcacatgatggtgatcttccgcctcatgcggacgaacttcctgatcaagttcctgctcatccaccagggcatgcacatggtcgcgggccacgacgccaacgacgccgtgatcagcaactccgtggcccaggcccgcttcagcgggctgctgatcgtcaagaccgtgctcgaccacatcctgcagaagaccgagcggggcgtgcgcctgcaccccctcgcgcggaccgccaaggtcaagaacgaggtgaactccttcaaggccgccctgagctccctggccaagcacggggagtacgcgcccttcgcccgcctcctgaacctgagcggcgtgaacaacctcgagcacggcctgttcccgcagctgtccgccatcgccctcggggtcgccacggcgcacggcagcaccctggccggggtgaacgtcggcgagcagtaccagcagctgcgggaggccgccaccgaggcggagaagcagctccagcagtacgccgagagccgcgagctggaccacctggggctcgacgaccaggagaagaagatcctgatgaacttccaccagaagaagaacgagatctccttccagcagaccaacgccatggtgacgctgcggaaggagcgcctggccaagctcaccgaggccatcaccgcggccagcctgcccaagacctccggccactacgacgacgacgacgacatccccttccccggcccgatcaacgacgacgacaaccccgggcaccaggacgacgaccccacggacagccaggacaccaccatccccgacgtggtcgtggacccggacgacggctcctacggggagtaccagagctactccgagaacggcatgaacgcccccgacgacctggtgctcttcgacctggacgaggacgacgaggacaccaagcccgtccccaaccggagcacgaagggcgggcagcagaagaactcccagaagggccagcacatcgaggggcgccagacccagagccggccgatccagaacgtgcccggcccccaccgcaccatccaccacgcctccgccccgctgaccgacaacgaccgccggaacgagcccagcgggtccacgagcccccgcatgctcacccccatcaacgaggaggcggaccccctggacgacgccgacgacgagacctccagcctgccgcccctcgagtccgacgacgaggagcaggaccgggacggcaccagcaaccgcacgcccaccgtggccccgcccgcccccgtctaccgggaccactccgagaagaaggagctgccccaggacgagcagcaggaccaggaccacacccaggaggcccgcaaccaggacagcgacaacacccagagcgagcactccttcgaggagatgtaccggcacatcctgcgcagccaggggccgttcgacgcggtgctctactaccacatgatgaaggacgagcccgtggtcttctccacgagcgacggcaaggagtacacctaccccgactccctggaggaggagtacccgccgtggctgaccgagaaggaggccatgaacgaggagaaccggttcgtgaccctcgacggccagcagttctactggcccgtgatgaaccacaagaacaagttcatggccatcctgcagcaccaccagtga

SEQ ID NO: 78 - RNA, 2220 nt, synthetic construct
  misc_feature 1..2220: note = EBOV NP, Sierra Leone 2014, optimized mRNA Sequence
  source 1..2220: mol_type = other RNA
atggacagccgcccccagaaggtgtggatgaccccgtccctgaccgagagcgacatggactaccacaagatcctcacggccggcctgtccgtccagcaggggatcgtgcggcagcgcgtgatccccgtctaccaggtgaacaacctggaggagatctgccagctcatcatccaggccttcgaggcgggcgtggacttccaggagagcgccgactccttcctgctgatgctctgcctgcaccacgcctaccagggggactacaagctgttcctcgagagcggcgccgtcaagtacctggaggggcacggcttccggttcgaggtgaagaagtgcgacggcgtgaagcgcctggaggagctcctgcccgccgtctccagcgggcggaacatcaagcgcaccctggcggccatgcccgaggaggagaccaccgaggccaacgccggccagttcctctccttcgcgagcctgttcctgccgaagctcgtggtgggggagaaggcctgcctggagaaggtccagcggcagatccaggtgcacgccgagcagggcctgatccagtaccccacggcctggcagtccgtggggcacatgatggtcatcttccgcctcatgcggaccaacttcctgatcaagttcctgctcatccaccagggcatgcacatggtggccggccacgacgcgaacgacgccgtgatcagcaactccgtcgcccaggcccgcttcagcgggctgctgatcgtgaagaccgtgctcgaccacatcctgcagaagaccgagcggggcgtccgcctgcaccccctcgcccggacggcgaaggtgaagaacgaggtgaactccttcaaggccgccctgagctccctggccaagcacggggagtacgcccccttcgcgcgcctcctgaacctgagcggcgtcaacaacctcgagcacggcctgttcccgcagctgtccgccatcgccctcggggtggccaccgcccacggcagcaccctggcgggggtcaacgtgggcgagcagtaccagcagctgcgggaggccgccaccgaggccgagaagcagctccagcagtacgcggagagccgcgagctggaccacctggggctcgacgaccaggagaagaagatcctgatgaacttccaccagaagaagaacgagatctccttccagcagacgaacgccatggtgaccctgcggaaggagcgcctggccaagctcaccgaggccatcaccgccgcgagcctgcccaagacgtccggccactacgacgacgacgacgacatccccttccccggcccgatcaacgacgacgacaaccccgggcaccaggacgacgaccccaccgacagccaggacaccaccatccccgacgtcgtggtggacccggacgacggcgggtacggcgagtaccagtcctacagcgagaacgggatgtccgcccccgacgacctggtcctcttcgacctggacgaggacgacgaggacacgaagcccgtgcccaaccggagcaccaagggcggccagcagaagaactcccagaaggggcagcacaccgagggccgccagacccagagcacgccgacccagaacgtgaccgggccccggcgcaccatccaccacgcctccgccccgctgacggacaacgaccgccggaacgagcccagcggctccaccagcccgcgcatgctcacccccatcaacgaggaggccgaccccctggacgacgcggacgacgagacctccagcctgcccccgctcgagtccgacgacgaggagcaggaccgggacgggacgagcaaccgcacccccaccgtcgccccgcccgcccccgtgtaccgggaccactccgagaagaaggagctgccccaggacgagcagcaggaccaggaccacatccaggaggcccgcaaccaggacagcgacaacacccagcccgagcacagcttcgaggagatgtaccggcacatcctgcgctcccagggcccgttcgacgccgtgctctactaccacatgatgaaggacgagcccgtcgtgttcagcacgtccgacggcaaggagtacacctaccccgacagcctggaggaggagtacccgccgtggctgaccgagaaggaggcgatgaacgacgagaaccggttcgtgaccctcgacgggcagcagttctactggcccgtcatgaaccaccgcaacaagttcatggccatcctgcagcaccaccagtga

SEQ ID NO: 79 - RNA, 2031 nt, synthetic construct
  misc_feature 1..2031: note = BDBV GP, Uganda 2007,optimized mRNA Sequence
  source 1..2031: mol_type = other RNA
atggtgaccagcggcatcctgcagctcccccgcgagcggttccgcaagacctccttcttcgtctgggtgatcatcctgttccacaaggtgttcccgatccccctgggggtcgtgcacaacaacacgctccaggtgagcgacatcgacaagctggtctgccgggacaagctgtccagcacctcccagctcaagagcgtgggcctgaacctggaggggaacggcgtggccaccgacgtccccaccgccacgaagcgctgggggttccgggcgggcgtgcccccgaaggtggtcaactacgaggccggcgagtgggccgagaactgctacaacctcgacatcaagaaggccgacgggtccgagtgcctgcccgaggcccccgagggcgtgcgcgggttcccccggtgccgctacgtgcacaaggtcagcggcaccgggccgtgccccgagggctacgcgttccacaaggagggcgccttcttcctgtacgaccggctcgcctccaccatcatctaccgcagcaccacgttctccgagggggtggtggccttcctgatcctgcccgagaccaagaaggacttcttccagagcccgcccctccacgagcccgcgaacatgaccaccgacccctccagctactaccacacggtcaccctgaactacgtggccgacaacttcggcaccaacatgaccaacttcctgttccaggtggaccacctcacgtacgtccagctggagccccggttcaccccgcagttcctggtgcagctcaacgagaccatctacaccaacgggcgccggtccaacacgaccggcaccctgatctggaaggtgaaccccaccgtcgacacgggcgtgggggagtgggccttctgggagaacaagaagaacttcaccaagaccctgagctccgaggagctcagcgtcatcttcgtgccccgcgcccaggaccccggctccaaccagaagaccaaggtgacgccgaccagcttcgccaacaaccagaccagcaagaaccacgaggacctggtccccgaggaccccgcgtccgtggtgcaggtccgggacctgcagcgcgagaacaccgtgcccacgcccccgcccgacaccgtgcccaccaccctcatcccggacacgatggaggagcagaccaccagccactacgagccgcccaacatctcccggaaccaccaggagcgcaacaacaccgcccaccccgagaccctggccaacaacccgcccgacaacacgacccccagcaccccgccccaggacggggagcggacctccagccacacgaccccgtccccgcgccccgtccccaccagcaccatccaccccacgacccgggagacccacatccccaccacgatgaccacctcccacgacaccgacagcaaccgcccgaaccccatcgacatctccgagagcacggagcccggccccctgaccaacaccacccgcggggccgccaacctcctgacgggctcccggcgcacccggcgcgagatcaccctgcggacccaggcgaagtgcaacccgaacctccactactggacgacccaggacgagggcgccgccatcgggctggcctggatcccctacttcggccccgccgcggaggggatctacaccgagggcatcatgcacaaccagaacgggctgatctgcggcctccgccagctggccaacgagaccacgcaggccctgcagctcttcctgcgggccaccaccgagctgcgcaccttcagcatcctcaaccggaaggccatcgacttcctgctgcagcgctggggcgggacgtgccacatcctcggccccgactgctgcatcgagccgcacgactggaccaagaacatcaccgacaagatcgaccagatcatccacgacttcatcgacaagcccctgcccgaccagaccgacaacgacaactggtggacggggtggcggcagtgggtgcccgcgggcatcgggatcaccggcgtgatcatcgccgtcatcgccctgctgtgcatctgcaagttcctcctgtga

SEQ ID NO: 80 - RNA, 2031 nt, synthetic construct
  misc_feature 1..2031: note = SUDV GP, Gulu, Uganda 2007, optimized mRNA Sequence
  source 1..2031: mol_type = other RNA
atgggcgggctgagcctcctgcagctgccccgcgacaagttccggaagtccagcttcttcgtgtgggtcatcatcctcttccagaaggccttctccatgccgctgggcgtggtgaccaacagcaccctggaggtcacggagatcgaccagctcgtgtgcaaggaccacctggcctccaccgaccagctgaagagcgtggggctcaacctggagggctccggggtcagcaccgacatcccctccgcgaccaagcgctggggcttccggagcggcgtgccgcccaaggtggtctcctacgaggccggggagtgggccgagaactgctacaacctggagatcaagaagcccgacggcagcgagtgcctcccgccgcccccggacggggtgcgcggcttcccccggtgccgctacgtgcacaaggcccaggggacgggcccctgccccggcgactacgccttccacaaggacggggcgttcttcctgtacgaccggctggcctccaccgtcatctaccgcggcgtgaacttcgccgagggggtgatcgccttcctcatcctggcgaagcccaaggagaccttcctgcagagcccgcccatccgggaggccgtcaactacaccgagaacacgtccagctactacgccacctcctacctcgagtacgagatcgagaacttcggcgcccagcacagcaccaccctgttcaagatcgacaacaacacgttcgtgcgcctggaccggccccacaccccgcagttcctcttccagctgaacgacaccatccacctgcaccagcagctctccaacaccacgggccgcctgatctggaccctggacgccaacatcaacgcggacatcggggagtgggccttctgggagaacaagaagaacctcagcgagcagctgcggggcgaggagctgagcttcgaggccctctccctgaacgagaccgaggacgacgacgccgccagctcccgcatcaccaaggggcggatcagcgaccgcgcgacgcggaagtactccgacctggtgcccaagaacagccccggcatggtccccctccacatcccggagggggagaccaccctgccctcccagaacagcaccgagggccgccgggtgggcgtgaacacgcaggagaccatcaccgagaccgccgccacgatcatcgggaccaacggcaaccacatgcagatctccaccatcgggatccgccccagctccagccagatcccctccagctccccgaccacggcccccagccccgaggcccagacccccaccacccacacgtccggccccagcgtcatggcgaccgaggagcccaccaccccgccggggtccagccccggccccaccacggaggcccccaccctgaccaccccggagaacatcacgaccgccgtgaagaccgtgctcccccaggagagcacctccaacggcctgatcacgagcaccgtcaccgggatcctgggctccctcgggctgcggaagcgcagccgccggcagaccaacacgaaggccaccggcaagtgcaaccccaacctgcactactggaccgcccaggagcagcacaacgcggccgggatcgcctggatcccctacttcggcccgggcgccgaggggatctacaccgagggcctcatgcacaaccagaacgcgctggtgtgcgggctgcgccagctcgccaacgagacgacccaggccctgcagctgttcctgcgggccaccaccgagctccgcacgtacaccatcctgaaccggaaggccatcgacttcctgctccgccggtggggcggcacctgccgcatcctggggcccgactgctgcatcgagccccacgactggaccaagaacatcacggacaagatcaaccagatcatccacgacttcatcgacaaccccctgccgaaccaggacaacgacgacaactggtggaccggctggcggcagtggatccccgcggggatcggcatcaccgggatcatcatcgccatcatcgccctcctgtgcgtctgcaagctgctctgctga

SEQ ID NO: 81 - RNA, 2031 nt, synthetic construct
  misc_feature 1..2031: note = TAFV GP, Cote dIvoire 1994, optimized mRNA Sequence
  source 1..2031: mol_type = other RNA
atgggcgccagcgggatcctgcagctcccccgcgagcggttccgcaagacctccttcttcgtgtgggtcatcatcctgttccacaaggtgttcagcatcccgctgggcgtggtccacaacaacaccctccaggtgtccgacatcgacaagttcgtgtgccgggacaagctgagctccacgagccagctgaagtccgtcgggctcaacctggagggcaacggggtggccaccgacgtgcccaccgcgaccaagcgctggggcttccgggccggcgtcccgcccaaggtggtgaactgcgaggccggggagtgggccgagaactgctacaacctggccatcaagaaggtcgacggcagcgagtgcctccccgaggcgcccgagggggtgcgcgacttcccccggtgccgctacgtgcacaaggtctccggcacggggccctgcccgggcggcctggccttccacaaggagggggccttcttcctgtacgaccggctcgccagcaccatcatctaccgcggcaccaccttcgcggagggggtgatcgccttcctgatcctgcccaaggcccggaaggacttcttccagtccccgcccctccacgagccggccaacatgacgaccgaccccagctcctactaccacaccaccacgatcaactacgtggtcgacaacttcggcaccaacaccaccgagttcctgttccaggtggaccacctgacgtacgtgcagctcgaggcccgcttcaccccgcagttcctggtcctgctcaacgagaccatctacagcgacaaccggcgctccaacaccacgggcaagctgatctggaagatcaaccccaccgtggacaccagcatgggggagtgggcgttctgggagaacaagaagaacttcaccaagacgctgtccagcgaggagctctccttcgtgcccgtccccgagacccagaaccaggtgctggacaccaccgccacggtcagcccgcccatcagcgcccacaaccacgccgccgaggaccacaaggagctggtgtccgaggacagcaccccggtggtccagatgcagaacatcaagggcaaggacaccatgcccaccacggtgaccggggtgcccaccaccacgccctccccgttccccatcaacgcgcggaacaccgaccacaccaagagcttcatcggcctcgaggggccccaggaggaccactccaccacccagcccgccaagacgaccagccagccgaccaactccaccgagagcacgaccctgaaccccacctccgagcccagctcccgcggcaccggcccgagctcccccacggtccccaacaccaccgagagccacgccgagctggggaagaccacgcccaccaccctcccggagcagcacaccgccgcctccgcgatcccccgggccgtgcaccccgacgagctgagcggccccgggttcctgacgaacaccatccgcggcgtgaccaacctcctgaccgggtcccggcgcaagcggcgcgacgtcacgccgaacacccagcccaagtgcaaccccaacctgcactactggaccgccctcgacgagggcgccgccatcggcctggcgtggatcccctacttcgggccggccgccgagggcatctacaccgaggggatcatggagaaccagaacggcctgatctgcgggctccgccagctggccaacgagacgacccaggcgctgcagctcttcctgcgggccaccaccgagctgcgcacgttcagcatcctcaaccggaaggccatcgacttcctgctgcagcgctggggcggcacctgccacatcctggggcccgactgctgcatcgagccccaggactggaccaagaacatcaccgacaagatcgaccagatcatccacgacttcgtggacaacaacctccccaaccagaacgacggctccaactggtggacggggtggaagcagtgggtgccggccggcatcggcatcaccggggtcatcatcgccatcatcgcgctgctgtgcatctgcaagttcatgctctga

SEQ ID NO: 82 - RNA, 981 nt, synthetic construct
  misc_feature 1..981: note = BDBV VP40, Uganda 2007, optimized mRNA Sequence
  source 1..981: mol_type = other RNA
atgcgccgggccatcctgcccaccgccccgcccgagtacatcgaggcggtgtaccccatgcgcaccgtcagcacgtccatcaacagcaccgcctccggccccaacttcccggcccccgacgtgatgatgagcgacaccccctccaacagcctccggcccatcgccgacgacaacatcgaccacccgtcccacacccccacgagcgtgtccagcgccttcatcctggaggcgatggtcaacgtgatctccgggcccaaggtgctgatgaagcagatccccatctggctcccgctgggcgtcgccgaccagaagacctacagcttcgactccaccaccgccgccatcatgctggcgagctacacgatcacccacttcgggaagacctccaaccccctcgtgcgcatcaaccggctgggccccgggatcccggaccacccgctgcgcctcctgcggatcggcaaccaggccttcctgcaggagttcgtgctcccgcccgtccagctgccccagtacttcaccttcgacctgacggccctcaagctgatcacccagcccctgcccgccgccacctggaccgacgacacgcccaccggccccaccgggatcctccgcccgggcatcagcttccaccccaagctgcggcccatcctgctccccgggaagaccggcaagcgcgggtccagctccgacctgacgagcccggacaagatccaggcgatcatgaacttcctgcaggacctcaagctggtgcccatcgaccccgccaagaacatcatgggcatcgaggtgccggagctgctcgtccaccggctgaccggcaagaagatcaccaccaagaacgggcagcccatcatccccatcctgctccccaagtacatcggcatggaccccatcagccagggggacctgacgatggtgatcacccaggactgcgacacctgccactcccccgccagcctgccgcccgtgtccgagaagtga

SEQ ID NO: 83 - RNA, 981 nt, synthetic construct
  misc_feature 1..981: note = SUDV VP40, Gulu, Uganda 2000, optimized mRNA Sequence
  source 1..981: mol_type = other RNA
atgcgccgggtgaccgtccccaccgccccgcccgcctacgcggacatcggctaccccatgagcatgctgcccatcaagtccagccgcgccgtgtccgggatccagcagaagcaggaggtgctcccgggcatggacacgcccagcaactccatgcggcccgtcgccgacgacaacatcgaccacaccagccacacccccaacggggtggcctccgccttcatcctggaggcgaccgtgaacgtcatcagcggcccgaaggtgctgatgaagcagatccccatctggctccccctggggatcgccgaccagaagacgtactccttcgacagcaccaccgccgccatcatgctggcgtcctacaccatcacgcacttcggcaaggccaacaaccccctcgtgcgcgtcaaccggctgggccaggggatcccggaccacccgctgcgcctcctgcggatgggcaaccaggccttcctgcaggagttcgtgctcccgcccgtgcagctgccccagtacttcaccttcgacctgaccgccctcaagctggtcacccagcccctgcccgccgcgacgtggaccgacgagacccccagcaacctctccggggccctgcgcccgggcctgagcttccaccccaagctccggcccgtgctgctgcccgggaagaccggcaagaagggccacgtgtccgacctcacggccccggacaagatccagaccatcgtcaacctgatgcaggacttcaagatcgtgcccatcgaccccgccaagagcatcatcgggatcgaggtgcccgagctgctcgtccacaagctgaccggcaagaagatgtcccagaagaacgggcagccgatcatccccgtgctgctccccaagtacatcggcctggacccgatcagccccggcgacctgaccatggtgatcacgcccgactacgacgactgccactcccccgccagctgcagctacctctccgagaagtga

SEQ ID NO: 84 - RNA, 981 nt, synthetic construct
  misc_feature 1..981: note = TAFV VP40, Cote dIvoire 1994, optimized mRNA Sequence
  source 1..981: mol_type = other RNA
atgcgccggatcatcctgcccaccgccccgcccgagtacatggaggccgtgtaccccatgcgcaccatgaacagcggcgcggacaacacggcctccgggcccaactacaccaccaccggcgtcatgacgaacgacaccccgagcaactccctccggcccgtggccgacgacaacatcgaccaccccagccacacccccaactccgtggccagcgccttcatcctggaggcgatggtcaacgtgatctccgggccgaaggtgctgatgaagcagatccccatctggctccccctgggcgtcagcgaccagaagacctactccttcgacagcacgaccgccgccatcatgctggcctcctacaccatcacccacttcgggaagacgagcaaccccctcgtgcgcatcaaccggctgggcccgggcatccccgaccaccccctgcgcctcctgcggatcgggaaccaggcgttcctgcaggagttcgtgctcccgcccgtccagctgccccagtacttcaccttcgacctgaccgccctcaagctgatcacccagcccctgcccgccgccacgtggaccgacgagaccccggccgtgtccaccggcacgctccgccccgggatcagcttccaccccaagctgcggcccatcctgctcccgggccgcgcggggaagaagggctccaacagcgacctgacctcccccgacaagatccaggccatcatgaacttcctgcaggacctcaagatcgtgcccatcgaccccaccaagaacatcatgggcatcgaggtcccggagctgctggtgcaccggctcaccgggaagaagacgaccaccaagaacggccagcccatcatccccatcctgctgccgaagtacatcgggctcgaccccctgagccagggcgacctgaccatggtgatcacgcaggactgcgactcctgccacagccccgccagcctcccgccggtcaacgagaagtga

SEQ ID NO: 85 - RNA, 2088 nt, synthetic construct
  misc_feature 1..2088: note = MARV NP, Angola 2005, optimized mRNA Sequence
  source 1..2088: mol_type = other RNA
atggacctgcacagcctcctggagctgggcaccaagcccaccgccccgcacgtgcgcaacaagaaggtcatcctcttcgacacgaaccaccaggtgtccatctgcaaccagatcatcgacgccatcaacagcgggatcgacctgggcgacctgctcgagggcgggctgctgaccctctgcgtggagcactactacaactccgacaaggacaagttcaacaccagccccatcgcgaagtacctgcgggacgccgggtacgagttcgacgtcatcaagaacgccgacgccacccgcttcctggacgtgatccccaacgagccccactactccccgctcatcctggccctgaagacgctcgagagcaccgagtcccagcggggccgcatcggcctgttcctgagcttctgctccctcttcctgcccaagctggtggtcggggaccgggcgagcatcgagaaggccctccgccaggtgaccgtgcaccaggagcagggcatcgtcacctaccccaaccactggctgacgaccgggcacatgaaggtgatcttcggcatcctgcggtccagcttcatcctcaagttcgtgctgatccaccagggggtcaacctggtgaccggccacgacgcctacgactccatcatcagcaactccgtgggccagacccgcttcagcgggctcctgatcgtcaagacggtgctggagttcatcctccagaagaccgactccggcgtgaccctgcaccccctggtccggaccagcaaggtgaagaacgaggtggcctccttcaagcaggcgctcagcaacctggcccgccacggggagtacgccccgttcgcccgggtcctgaacctcagcggcatcaacaacctggagcacggcctgtacccccagctctccgccatcgcgctgggggtggccacggcccacggcagcaccctggccggggtgaacgtcggcgagcagtaccagcagctccgcgaggccgcgcacgacgccgaggtgaagctgcagcggcgccacgagcaccaggagatccaggccatcgccgaggacgacgaggagcggaagatcctggagcagttccacctccagaagaccgagatcacccactcccagacgctggccgtcctgagccagaagcgcgagaagctggcgcggctcgccgccgagatcgagaacaacatcgtggaggaccaggggttcaagcagtcccagaaccgcgtgagccagtccttcctgaacgaccccacccccgtcgaggtgaccgtgcaggcccggccgatcaaccgccccaccgccctgcccccgcccgtcgacagcaagatcgagcacgagtccacggaggacagctccagctccagctccttcgtggacctcaacgaccccttcgcgctgctgaacgaggacgaggacaccctcgacgacagcgtgatgatcccctccaccaccagccgggagttccagggcatccccgagccgccccgccagtcccaggacatcgacaacagccagggcaagcaggaggacgagagcacgaacctgatcaagaagcccttcctgcgctaccaggagctcccgccggtccaggaggacgacgagtccgagtacaccaccgacagccaggagtccatcgaccagcccgggagcgacaacgagcagggcgtggacctgccgcccccgcccctgtacgcccaggagaagcggcaggaccccatccagcacccggccgtgtccagccaggaccccttcgggtccatcggcgacgtcaacggggacatcctcgagcccatccgcagcccctccagcccctccgccccgcaggaggacacccgggcgcgcgaggcctacgagctgagccccgacttcacgaactacgaggacaaccagcagaactggccgcagcgggtggtgaccaagaagggccgcaccttcctgtacccgaacgacctcctgcagaccaacccgcccgagtccctgatcacggccctcgtcgaggagtaccagaaccccgtgagcgccaaggagctgcaggccgactggcccgacatgtccttcgacgagcggcgccacgtggcgatgaacctgtga

SEQ ID NO: 86 - RNA, 2220 nt, synthetic construct
  misc_feature 1..2220: note = BDBV NP, Uganda 2007, optimized mRNA Sequence
  source 1..2220: mol_type = other RNA
atggacccccgcccgatccggacctggatgatgcacaacaccagcgaggtggaggccgactaccacaagatcctgacggccggcctctccgtccagcaggggatcgtgcgccagcggatcatccccgtgtaccagatcagcaacctggaggaggtctgccagctgatcatccaggcgttcgaggccggcgtggacttccaggactccgccgacagcttcctcctgatgctgtgcctccaccacgcctaccagggggactacaagcagttcctggagtccaacgccgtgaagtacctggagggccacgggttccgcttcgagatgaagaagaaggagggcgtcaagcggctcgaggagctgctgcccgcggccagctccggcaagaacatcaagcgcaccctcgccgccatgcccgaggaggagaccaccgaggcgaacgccgggcagttcctgagcttcgcctccctgttcctcccgaagctggtggtgggcgagaaggcctgcctggagaaggtccagcggcagatccaggtgcacgccgagcaggggctcatccagtaccccacgagctggcagtccgtgggccacatgatggtcatcttccgcctgatgcggaccaacttcctgatcaagttcctcctgatccaccaggggatgcacatggtggcgggccacgacgccaacgacgccgtgatcgccaacagcgtcgcccaggcgcgcttctccggcctgctcatcgtgaagaccgtgctggaccacatcctgcagaagaccgagcacggggtccggctccaccccctggcccgcacggccaaggtgaagaacgaggtgagctccttcaaggccgccctggcgagcctcgcccagcacggcgagtacgcccccttcgcccggctgctgaacctctccggggtcaacaacctggagcacggcctgttcccgcagctcagcgccatcgcgctgggcgtggccaccgcccacgggtccaccctggccggcgtgaacgtcggggagcagtaccagcagctccgcgaggcggccaccgaggccgagaagcagctgcagaagtacgccgagagccgggagctggaccacctcggcctggacgaccaggagaagaagatcctgaaggacttccaccagaagaagaacgagatcagcttccagcagacgaccgccatggtgaccctccgcaaggagcggctggcgaagctgaccgaggccatcacgtccaccagcatcctgaagaccgggcgccggtacgacgacgacaacgacatccccttccccggccccatcaacgacaacgagaactccggccagaacgacgacgacccgaccgacagccaggacacgaccatccccgacgtcatcatcgaccccaacgacgggggctacaacaactactccgactacgccaacgacgccgccagcgcgcccgacgacctcgtgctgttcgacctggaggacgaggacgacgccgacaacccggcccagaacacccccgagaagaacgaccgccccgccaccacgaagctccggaacgggcaggaccaggacggcaaccagggggagaccgcctccccgcgcgtggcgcccaaccagtaccgcgacaagcccatgccccaggtccaggaccggagcgagaaccacgaccagaccctgcagacccagtcccgcgtgctgacgcccatcagcgaggaggccgacccgtccgaccacaacgacggcgacaacgagagcatcccgcccctcgagtccgacgacgagggcagcaccgacaccaccgccgccgagacgaagcccgcgaccgccccgcccgcccccgtgtaccggtccatcagcgtcgacgactccgtgccgagcgagaacatccccgcccagtccaaccagaccaacaacgaggacaacgtgcgcaacaacgcccagagcgagcagagcatcgcggagatgtaccagcacatcctgaagacccaggggcccttcgacgccatcctgtactaccacatgatgaaggaggagccgatcatcttctccacgagcgacggcaaggagtacacctaccccgactccctcgaggacgagtacccgccctggctgagcgagaaggaggccatgaacgaggacaaccggttcatcaccatggacgggcagcagttctactggcccgtcatgaaccaccgcaacaagttcatggccatcctgcagcaccaccggtga

SEQ ID NO: 87 - RNA, 2217 nt, synthetic construct
  misc_feature 1..2217: note = SUDV NP, Gulu, Uganda 2000,optimized mRNA Sequence
  source 1..2217: mol_type = other RNA
atggacaagcgcgtgcggggcagctgggccctgggcgggcagtccgaggtcgacctcgactaccacaagatcctgaccgccgggctgagcgtgcagcagggcatcgtgcgccagcgggtcatccccgtgtacgtggtctccgacctcgaggggatctgccagcacatcatccaggcgttcgaggccggcgtggacttccaggacaacgccgacagcttcctgctgctcctgtgcctgcaccacgcctaccagggcgaccaccgcctcttcctgaagtccgacgccgtgcagtacctggaggggcacggcttccggttcgaggtccgcgagaaggagaacgtgcaccggctcgacgagctgctgccgaacgtgaccgggggcaagaacctccgccggacgctggcggccatgcccgaggaggagaccaccgaggccaacgccgggcagttcctgagcttcgcgtccctcttcctgcccaagctggtcgtgggcgagaaggcctgcctcgagaaggtgcagcgccagatccaggtccacgccgagcagggcctgatccagtaccccaccagctggcagtccgtggggcacatgatggtgatcttccggctgatgcgcacgaacttcctcatcaagttcctgctgatccaccagggcatgcacatggtcgccgggcacgacgccaacgacaccgtgatcagcaactccgtggcgcaggcccggttcagcggcctcctgatcgtcaagaccgtgctggaccacatcctccagaagaccgacctgggcgtgcgcctgcacccgctcgcccggacggccaaggtcaagaacgaggtgtccagcttcaaggccgcgctggggtccctggccaagcacggcgagtacgcccccttcgcccgcctcctgaacctgagcggggtcaacaacctcgagcacggcctgtacccccagctgtccgccatcgcgctcggggtggccaccgcccacggcagcaccctggccggcgtgaacgtcggggagcagtaccagcagctgcgggaggccgcgaccgaggccgagaagcagctccagcagtacgccgagacgcgcgagctggacaacctgggcctggacgagcaggagaagaagatcctcatgtccttccaccagaagaagaacgagatcagcttccagcagaccaacgccatggtgaccctgcggaaggagcgcctggcgaagctcaccgaggccatcacgaccgccagcaagatcaaggtgggggaccggtaccccgacgacaacgacatcccgttccccggccccatctacgacgagacccaccccaacccgtccgacgacaaccccgacgacagccgcgacaccacgatccccgggggcgtcgtggacccctacgacgacgagtccaacaactacccggactacgaggacagcgccgagggcaccaccggggacctggacctgttcaacctcgacgacgacgacgacgactcccagcccggcccgcccgaccgcgggcagagcaaggagcgggccgcgcgcacccacggcctgcaggaccccacgctggacggggccaagaaggtgcccgagctcacccccggctcccaccagcccggcaacctgcacatcaccaagccggggagcaacaccaaccagccccagggcaacatgtccagcacgctgcagtccatgacccccatccaggaggagagcgagcccgacgaccagaaggacgacgacgacgagtccctcaccagcctggactccgagggggacgaggacgtcgagagcgtgtccggcgagaacaacccgaccgtggccccgcccgccccggtctacaaggacacgggcgtggacaccaaccagcagaacgggcccagcaacgccgtggacggccaggggtccgagagcgaggcgctgcccatcaaccccgagaagggcagcgccctcgaggagacctactaccacctgctgaagacccaggggccgttcgaggccatcaactactaccacctcatgtccgacgagcccatcgccttcagcacggagtccggcaaggagtacatcttcccggacagcctggaggaggcctacccgccctggctgtccgagaaggaggcgctcgagaaggagaaccggtacctggtcatcgacggccagcagttcctgtggcccgtgatgagcctccaggacaagttcctggccgtgctgcagcacgactga

SEQ ID NO: 88 - RNA, 2220 nt, synthetic construct
  misc_feature 1..2220: note = TAFV NP, Cote dIvoire 1994,optimized mRNA Sequence
  source 1..2220: mol_type = other RNA
atggagagccgcgcccacaaggcctggatgacccacaccgcgtccggcttcgagacggactaccacaagatcctgaccgccgggctcagcgtgcagcagggcatcgtccggcagcgcgtgatccaggtgcaccaggtcaccaacctggaggagatctgccagctgatcatccaggccttcgaggccggggtggacttccaggagtccgccgacagcttcctcctgatgctgtgcctccaccacgcgtaccagggcgactacaagcagttcctggagtccaacgccgtgaagtacctggaggggcacggcttccggttcgaggtccgcaagaaggagggcgtgaagcggctcgaggagctgctgcccgccgccagctccgggaagagcatccgccggaccctcgcggccatgccggaggaggagacgaccgaggccaacgccggccagttcctgtccttcgccagcctgttcctccccaagctggtggtcggggagaaggcgtgcctggagaaggtgcagcgccagatccaggtgcactccgagcagggcctcatccagtaccccaccgcctggcagagcgtcgggcacatgatggtgatcttccggctgatgcgcaccaacttcctgatcaagttcctcctgatccaccagggcatgcacatggtggccggccacgacgccaacgacgccgtcatcgcgaactccgtggcccaggcccggttcagcgggctgctcatcgtgaagacggtcctggaccacatcctgcagaagaccgagcacggcgtgcgcctccaccccctggcccggaccgccaaggtgaagaacgaggtcaactccttcaaggcggccctgagctccctcgcccagcacggggagtacgccccgttcgcccgcctgctgaacctcagcggcgtgaacaacctggagcacggcctgttcccccagctctccgcgatcgccctgggggtggccaccgcccacggcagcacgctggcgggggtcaacgtgggcgagcagtaccagcagctccgggaggccgccaccgaggccgagaagcagctgcagaagtacgccgagagccgcgagctggaccacctcgggctggacgaccaggagaagaagatcctgaaggacttccaccagaagaagaacgagatctccttccagcagaccaccgcgatggtcacgctccggaaggagcgcctggccaagctgaccgaggccatcaccagcacctccctgctcaagacgggcaagcagtacgacgacgacaacgacatccccttccccggcccgatcaacgacaacgagaacagcgagcagcaggacgacgaccccaccgactcccaggacaccaccatccccgacatcatcgtggaccccgacgacgggcggtacaacaactacggcgactacccgagcgagacggccaacgcccccgaggacctggtgctgttcgacctcgaggacggggacgaggacgaccaccgcccctccagctccagcgagaacaacaacaagcactccctgaccggcaccgacagcaacaagacctccaactggaaccggaaccccacgaacatgccgaagaaggacagcacccagaacaacgacaaccccgcgcagcgcgcccaggagtacgcccgcgacaacatccaggacacccccaccccgcaccgggccctgacgcccatctccgaggagaccgggagcaacggccacaacgaggacgacatcgactccatcccgcccctcgagagcgacgaggagaacaacaccgagaccacgatcaccaccaccaagaacaccacggccccgcccgcgcccgtctaccgctccaacagcgagaaggagcccctgccccaggagaagagccagaagcagccgaaccaggtgtccggcagcgagaacaccgacaacaagccccactccgagcagagcgtggaggagatgtaccggcacatcctgcagacccaggggcccttcgacgccatcctctactactacatgatgaccgaggagccgatcgtcttctccacgagcgacggcaaggagtacgtgtaccccgactccctggagggggagcacccgccctggctgagcgagaaggaggccctcaacgaggacaaccgcttcatcaccatggacgaccagcagttctactggccggtgatgaaccaccggaacaagttcatggccatcctgcagcaccacaagtga

SEQ ID NO: 89 - RNA, 2031 nt, synthetic construct
  misc_feature 1..2031: note = EBOV GP, Mayinga, Zaire 1976, optimized mRNA Sequence
  source 1..2031: mol_type = other RNA
atgggcgtgacaggcatcctgcagctgccccgggaccggttcaagcggaccagcttcttcctgtgggtcatcatcctgttccagcggaccttcagcatccccctgggcgtgatccacaacagcaccctgcaggtgtccgacgtggacaagctcgtgtgccgggacaagctgagcagcaccaaccagctgagaagcgtgggcctgaacctggaaggcaatggcgtggccaccgatgtgcctagcgccaccaagagatggggcttcagatccggcgtgccccccaaggtcgtgaattatgaggccggcgagtgggccgagaactgctacaacctggaaatcaagaagcccgacggcagcgagtgcctgcctgctgctcctgatggcatcagaggcttcccccggtgcagatacgtgcacaaggtgtccggcacaggcccctgcgctggcgatttcgcctttcacaaagagggcgcctttttcctgtacgaccggctggcctccaccgtgatctacagaggcaccacctttgccgagggcgtggtggcctttctgatcctgcctcaggccaagaaggacttcttcagcagccaccccctgcgcgagcctgtgaatgccacagaggatcccagcagcggctactacagcaccaccatcagataccaggccaccggcttcggcaccaacgagacagagtacctgttcgaggtggacaacctgacctacgtgcagctggaaagccggttcaccccccagtttctgctgcagctgaacgagacaatctacaccagcggcaagcggagcaataccaccggcaagctgatctggaaagtgaaccccgagatcgataccacaatcggagagtgggccttctgggagacaaagaagaacctgacccggaagatcagaagcgaggaactgagcttcaccgtggtgtccaacggcgccaagaacatcagcggacagagccccgccagaaccagcagcgatcctggcaccaacaccaccaccgaggaccacaagatcatggccagcgagaacagcagcgccatggtgcaggtgcacagccagggaagagaagccgccgtgtctcacctgaccaccctggccacaatcagcacaagcccccagagcctgaccacaaagcctggccccgacaactccacccacaacacccccgtgtacaagctggacatcagcgaggccacccaggtggaacagcaccatcggagaaccgacaacgacagcaccgccagcgataccccatctgccacaactgccgccggacctcccaaggccgagaataccaacacctccaagagcaccgactttctggaccccgccaccacaaccagccctcagaaccactctgagacagccggcaacaacaatacccaccaccaggataccggcgaggaaagcgccagctctggcaagctgggactgatcaccaacacaatcgccggcgtggccggcctgattacaggcggcagaagaaccagacgcgaggccatcgtgaacgcccagcccaagtgcaaccccaacctgcactactggaccacccaggatgagggcgctgctatcggactggcctggatcccttactttggccctgccgccgagggcatctacatcgagggcctgatgcacaaccaggacggcctgatctgcggactgaggcagctggccaacgaaaccacacaggccctgcagctgttcctgcgggccaccacagagctgagaaccttctccatcctgaatcggaaggctatcgacttcctgctgcagcgctggggcggcacctgtcacattctgggccctgactgctgcatcgagccccacgactggaccaagaatatcaccgacaagatcgaccagatcatccacgactttgtggacaagaccctgcccgaccagggcgacaacgataactggtggacaggctggcggcagtggatccctgctggcatcggagtgaccggcgtgatcattgccgtgatcgccctgttctgcatctgcaagttcgtgttctga

SEQ ID NO: 90 - RNA, 2031 nt, synthetic construct
  misc_feature 1..2031: note = EBOV GP, Sierra Leone 2014, optimized mRNA Sequence
  source 1..2031: mol_type = other RNA
atgggcgtgacaggcatcctgcagctgccccgggaccggttcaagcggaccagcttcttcctgtgggtcatcatcctgttccagcggaccttcagcatccccctgggcgtgatccacaacagcaccctgcaggtgtccgacgtggacaagctcgtgtgccgggacaagctgagcagcaccaaccagctgagaagcgtgggcctgaacctggaaggcaatggcgtggccaccgatgtgcccagcgtgaccaagagatggggcttcagatccggcgtgccccccaaggtcgtgaattatgaggccggcgagtgggccgagaactgctacaacctggaaatcaagaagcccgacggcagcgagtgcctgcctgctgctcctgatggcatcagaggcttcccccggtgcagatacgtgcacaaggtgtccggcacaggcccctgcgctggcgatttcgcctttcacaaagagggcgcctttttcctgtacgaccggctggcctccaccgtgatctacagaggcaccacctttgccgagggcgtggtggcctttctgatcctgcctcaggccaagaaggacttcttcagcagccaccccctgcgcgagcctgtgaatgccacagaggatcccagcagcggctactacagcaccaccatcagataccaggccaccggcttcggcaccaacgagacagagtacctgttcgaggtggacaacctgacctacgtgcagctggaaagccggttcaccccccagtttctgctgcagctgaacgagacaatctacgccagcggcaagcggagcaacaccaccggcaagctgatctggaaagtgaaccccgagatcgataccacaatcggagagtgggccttctgggagacaaagaagaacctgacccggaagatcagaagcgaggaactgagcttcaccgccgtgtccaacggccccaagaacatcagcggacagagccccgccagaaccagcagcgaccccgagacaaacaccaccaatgaggaccacaagatcatggccagcgagaacagcagcgccatggtgcaggtgcacagccagggaagaaaggccgctgtgtcccacctgaccaccctggccacaatctccaccagccctcagccccccaccaccaagaccggccctgataactccacccacaacacccccgtgtacaagctggacatcagcgaggccacacaagtgggccagcaccacagaagggccgacaacgatagcaccgccagcgatacccctccagccacaactgctgccggacctctgaaggccgagaataccaacaccagcaagagcgccgacagcctggatctggccaccaccacaagcccccagaactactctgagacagccggcaacaacaacacccatcaccaggataccggcgaggaaagcgccagctctggcaagctgggactgatcaccaacacaatcgccggcgtggccggcctgattaccggggggagaagaaccagacgggaagtgatcgtgaacgcccagcccaagtgcaaccccaacctgcactactggaccacccaggatgagggcgctgctatcggactggcctggatcccttactttggccctgccgccgagggcatctacaccgagggactgatgcacaaccaggacggcctgatctgcggactgaggcagctggccaacgaaaccacacaggccctgcagctgttcctgagagccaccaccgagctgaggaccttctccatcctgaacagaaaggctatcgacttcctgctgcagcgctggggcggcacctgtcacattctgggccctgactgctgcatcgagccccacgactggaccaagaatatcaccgacaagatcgaccagatcatccacgactttgtggacaagaccctgcccgaccagggcgacaatgacaactggtggacaggctggcggcagtggattcctgccggcattggagtgaccggcgtgatcattgccgtgatcgccctgttctgcatctgcaagttcgtgttctga

SEQ ID NO: 91 - RNA, 2046 nt, synthetic construct
  misc_feature 1..2046: note = MARV GP, Angola 2005, optimized mRNA Sequence
  source 1..2046: mol_type = other RNA
atgaagaccacctgtctgctgatctccctgatcctgatccagggcgtgaaaaccctgcccatcctggaaatcgccagcaacatccagccccagaacgtggacagcgtgtgcagcggcaccctgcagaaaaccgaggacgtgcacctgatgggcttcaccctgagcggacagaaggtggccgacagccctctggaagccagcaagagatgggccttcagagccggcgtgccccccaagaatgtggagtacacagagggcgaggaagccaagacctgctacaacatcagcgtgaccgaccccagcggcaagagcctgctgctggacccccccaccaacatcagagactaccccaagtgcaagaccatccaccacatccagggccagaacccccacgcccagggaattgctctgcacctgtggggcgcctttttcctgtacgaccggatcgcctccaccaccatgtaccggggcaaggtgttcaccgagggcaatatcgccgccatgatcgtgaacaagaccgtgcacaagatgatcttcagcaggcagggccagggctaccggcacatgaatctgaccagcaccaacaagtactggaccagcagcaacggcacccagaccaacgacaccggctgcttcggaaccctgcaggagtacaacagcaccaagaaccagacctgcgcccccagcaagaagcccctgcctctgcctacagcccaccccgaagtgaagctgacctccaccagcaccgacgccaccaagctgaacaccaccgaccctaacagcgacgacgaggacctgaccacaagcggctctggatccggcgagcaggaaccctacaccacctctgacgccgccacaaagcagggcctgagcagcaccatgcctcccacacctagccctcagcctagcacacctcagcagggcggcaacaacaccaaccactctcagggcgtcgtgaccgagcccggcaagaccaataccacagcccagcccagcatgcccccccacaataccaccacaatctccaccaacaataccagcaagcacaacctgagcacccccagcgtgcccatccagaatgccaccaactacaacacccagagcaccgcccctgagaacgagcagacaagcgccccttccaagaccaccctgctgcccaccgagaatcctaccaccgccaagagcaccaatagcaccaagtcccccaccaccaccgtgcccaacaccacaaacaagtacagcacctcccccagccccaccccaaattctaccgcccagcacctggtgtacttccggcggaagcggaacatcctgtggcgcgagggcgatatgttccccttcctggacggcctgatcaacgcccccatcgacttcgaccccgtgcctaacacaaagaccatcttcgacgagagcagcagcagcggcgccagcgccgaagaagatcagcacgccagccccaatatcagcctgaccctgagctacttccccaaagtgaacgagaacaccgcccacagcggcgagaacgagaatgactgtgacgccgagctgcggatttggagcgtgcaggaagatgacctggccgctggcctgagctggatcccttttttcggccctggcatcgagggcctgtacaccgccggactgatcaagaatcagaacaacctcgtgtgccggctgcggcggctggccaatcagacagccaagtccctggaactgctgctgagagtgaccaccgaggaacggaccttctctctgatcaaccggcacgccatcgattttctgctggccagatggggcggcacatgcaaagtgctgggccccgattgctgcatcggcattgaggacctgtcccggaacatctccgagcagatcgaccagatcaagaaggacgagcagaaagagggcaccggctggggactgggcggaaagtggtggacatctgactggggagtgctgaccaacctgggcattcttctgctgctgtctatcgccgtgctgattgccctgagctgcatctgccggatcttcaccaagtacatcggctga

SEQ ID NO: 92 - RNA, 981 nt, synthetic construct
  misc_feature 1..981: note = EBOV VP40, Mayinga, Zaire 1976, optimized mRNA Sequence
  source 1..981: mol_type = other RNA
atgcggagagtgatcctgcctaccgcccctcccgagtacatggaagccatctaccccgtgcggagcaacagcacaatcgccagaggcggcaacagcaataccggcttcctgacccccgagagcgtgaacggcgatacccccagcaatcccctgaggcctatcgccgacgacaccatcgatcacgccagccacacacctggcagcgtgtccagcgccttcatcctggaagctatggtcaacgtgatcagcggccccaaggtgctgatgaagcagatccccatctggctgcctctgggcgtggccgaccagaaaacctacagcttcgacagcaccaccgccgccatcatgctggcctcttacaccatcacccacttcggcaaggccaccaaccccctggtgcgcgtgaacagactgggccctggaatccccgaccaccccctgagactgctgagaatcggcaaccaggcctttctgcaggaatttgtgctgccccccgtgcagctgccccagtacttcacctttgacctgaccgccctgaagctgatcacccagcctctgcctgccgccacctggaccgatgatacacccacaggcagcaacggcgctctgaggcctggcatcagcttccaccctaagctgcggcccatcctgctgcccaacaagagcggcaagaagggcaacagcgccgacctgacctcccccgagaagatccaggccatcatgaccagcctgcaggacttcaagatcgtgcccatcgaccccaccaagaacatcatgggcatcgaggtgcccgagacactggtgcacaagctgaccggcaagaaagtgaccagcaagaacggccagcccatcatccctgtgctgctgcctaagtacatcggcctggaccctgtggcccctggcgatctgaccatggtcatcacccaggactgcgatacctgccacagccctgcttctctgcccgccgtgatcgagaagtga

SEQ ID NO: 93 - RNA, 981 nt, synthetic construct
  misc_feature 1..981: note = EBOV VP40, Sierra Leone 2014,optimized mRNA Sequence
  source 1..981: mol_type = other RNA
atgcggagagtgatcctgcctaccgcccctcccgagtacatggaagccatctaccccgccagaagcaacagcacaatcgccagaggcggcaacagcaataccggcttcctgacccccgagagcgtgaacggcgatacccccagcaatcccctgaggcctatcgccgacgacaccatcgatcacgccagccacacacctggcagcgtgtccagcgccttcatcctggaagctatggtcaacgtgatcagcggccccaaggtgctgatgaagcagatccccatctggctgcctctgggcgtggccgaccagaaaacctacagcttcgacagcaccaccgccgccatcatgctggcctcttacaccatcacccacttcggcaaggccaccaaccccctggtgcgcgtgaacagactgggccctggaatccccgaccaccccctgagactgctgagaatcggcaaccaggcctttctgcaggaatttgtgctgccccccgtgcagctgccccagtacttcacctttgacctgaccgccctgaagctgatcacccagcctctgcctgccgccacctggaccgatgatacacccacaggcagcaacggcgctctgaggcctggcatcagcttccaccctaagctgcggcccatcctgctgcccaacaagagcggcaagaagggcaacagcgccgacctgacctcccccgagaagatccaggccatcatgaccagcctgcaggacttcaagatcgtgcccatcgaccccaccaagaacatcatgggcatcgaggtgcccgagacactggtgcacaagctgaccggcaagaaagtgaccagcaagaacggccagcccatcatccctgtgctgctgcctaagtacatcggcctggaccctgtggcccctggcgatctgaccatggtcatcacccaggactgcgatacctgccacagccctgcttctctgcccgccgtggtggaaaagtga

SEQ ID NO: 94 - RNA, 912 nt, synthetic construct
  misc_feature 1..912: note = MARV VP40, Angola 2005,optimized mRNA Sequence
  source 1..912: mol_type = other RNA
atggccagcagcagcaactacaacacctacatgcagtacctgaacccccctccctacgccgaccatggcgccaaccagctgattcccgccgaccagctgtccaaccagcagggcatcacccccaactacgtgggcgacctgaacctggacgaccagttcaagggcaacgtgtgccacgccttcaccctggaagccatcatcgacatcagcgcctacaacgagcggaccgtgaaaggcgtgccagcctggctgcctctgggcatcatgagcaacttcgagtaccccctggcccacacagtggccgctctgctgacaggcagctacaccatcacccagttcacccacaacggccagaaattcgtcagagtgaaccggctgggcaccggcattccagcccaccctctgagaatgctgagagagggcaaccaggccttcatccagaacatggtcatcccccggaacttcagcaccaatcagtttacctacaacctgaccaacctggtgctgagcgtgcagaagctgcctgacgacgcttggaggcccagcaaggacaagctgatcggcaacaccatgcaccccgccgtgtccgtgcaccctaatctgcctccaatcgtgctgcccaccgtgaagaagcaggcctaccggcagcacaagaaccccaacaacggacccctgctggctatcagcggcatcctgcaccagctgcgggtggaaaaggtgcccgagaaaaccagcctgttccggatcagcctgcccgccgacatgttcagcgtgaaagagggcatgatgaagaagcggggcgagaacagccccgtggtgtattttcaagcccccgagaatttccccctgaacggcttcaacaacagacaggtggtgctggcctacgccaaccctacactgagcgccgtgtga

SEQ ID NO: 95 - RNA, 2220 nt, synthetic construct
  misc_feature 1..2220: note = EBOV NP, Zaire 1976, optimized mRNA Sequence
  source 1..2220: mol_type = other RNA
atggacagcagaccccagaaaatctggatggcccccagcctgaccgagagcgacatggactaccacaagatcctgacagccggcctgagcgtgcagcagggaattgtgcggcagcgcgtgatccccgtgtaccaagtgaacaacctggaagagatctgccagctgatcatccaggccttcgaggccggcgtggacttccaggaaagcgccgatagcttcctgctgatgctgtgcctgcaccacgcctaccagggcgactacaagctgttcctggaatctggcgccgtgaagtacctggaaggacacggcttcagattcgaagtgaagaaacgggacggcgtgaagcggctggaagaactgctgcctgccgtgtccagcggcaagaacatcaagagaaccctggccgccatgcccgaggaagagacaacagaggccaacgccggccagttcctgagcttcgccagcctgttcctgcccaagctggtcgtgggagagaaggcctgcctggaaaaggtgcagcggcagatccaggtgcacgccgagcagggcctgatccagtatcctacagcctggcagagcgtgggccacatgatggtcatcttccggctgatgaggaccaacttcctgatcaagtttctgctgatccaccagggcatgcacatggtggccggacacgacgccaacgacgccgtgatcagcaattctgtggcccaggccagattcagcggcctgctgatcgtgaaaaccgtgctggaccacatcctgcagaaaaccgagagaggcgtgcggctgcacccactggccagaaccgccaaagtgaaaaacgaagtgaacagcttcaaggccgccctgagcagcctggccaagcacggcgaatatgcccccttcgccagactgctgaacctgagcggcgtgaacaatctggaacacggcctgtttccccagctgagcgccattgctctgggagtggccacagcccacggaagcacactggctggcgtgaacgtgggcgagcagtaccagcagctgagagaggccgccacagaggccgagaaacagctgcagcagtacgccgagagcagagagctggatcacctgggcctggacgaccaggaaaagaaaatcctgatgaacttccaccagaagaagaacgagatcagcttccagcagaccaacgccatggtcaccctgcggaaagagcggctggccaaactgaccgaggccatcacagccgcctccctgcctaagacaagcggccactacgacgacgatgacgacatccccttccctggccccatcaatgacgacgacaacccaggccaccaggacgacgaccctaccgacagccaggacaccaccatccccgacgtggtggtggaccctgacgatggcagctacggcgagtaccagagctacagcgagaacggcatgaacgcccccgacgacctggtgctgttcgacctggacgaggacgatgaggataccaagcccgtgcccaacagaagcaccaagggcggccagcagaagaacagccagaagggccagcacatcgagggcagacagacccagagccggcccatccagaatgtgcctggcccccacagaaccatccaccatgcctctgcccccctgaccgacaacgacagaagaaacgagcccagcggcagcaccagccccagaatgctgacccccatcaacgaggaagccgaccccctggacgatgccgacgacgagacatctagcctgccccccctggaatccgatgacgaggaacaggacagggacggcaccagcaacagaacccctacagtggcccctcccgcccctgtgtacagagatcactccgagaagaaagagctgccccaggacgagcagcaggaccaggatcacacccaggaagcccggaaccaggacagcgacaacacccagagcgagcacagcttcgaggaaatgtaccggcacatcctgagatcccagggccccttcgatgccgtgctgtactatcacatgatgaaggacgagcccgtggtgttcagcacctccgacggcaaagagtacacctaccccgacagcctggaagaagagtaccccccttggctgacagagaaagaagccatgaacgaagagaatcgcttcgtgaccctggacggacagcagttttactggcccgtgatgaaccacaagaacaagttcatggccattctgcagcaccaccagtga

SEQ ID NO: 96 - RNA, 2220 nt, synthetic construct
  misc_feature 1..2220: note = EBOV NP, Sierra Leone 2014, optimized mRNA Sequence
  source 1..2220: mol_type = other RNA
atggacagcagaccccagaaagtgtggatgacccccagcctgaccgagagcgacatggactaccacaagatcctgacagccggcctgagcgtgcagcagggaattgtgcggcagcgcgtgatccccgtgtaccaagtgaacaacctggaagagatctgccagctgatcatccaggccttcgaggccggcgtggacttccaggaaagcgccgatagcttcctgctgatgctgtgcctgcaccacgcctaccagggcgactacaagctgttcctggaatctggcgccgtgaagtacctggaaggacacggcttcagattcgaagtgaagaaatgcgacggcgtgaagcggctggaagaactgctgcctgccgtgtccagcggccggaacatcaagagaacactggccgccatgcccgaggaagagacaaccgaagccaacgccggccagttcctgagcttcgccagcctgttcctgcccaagctggtcgtgggagagaaggcctgcctggaaaaggtgcagcggcagatccaggtgcacgccgagcagggcctgatccagtatcctacagcctggcagagcgtgggccacatgatggtcatcttccggctgatgaggaccaacttcctgatcaagtttctgctgatccaccagggcatgcacatggtggccggacacgacgccaacgacgccgtgatcagcaattctgtggcccaggccagattcagcggcctgctgatcgtgaaaaccgtgctggaccacatcctgcagaaaaccgagagaggcgtgcggctgcacccactggccagaaccgccaaagtgaaaaacgaagtgaacagcttcaaggccgccctgagcagcctggccaagcacggcgaatatgcccccttcgccagactgctgaacctgagcggcgtgaacaatctggaacacggcctgtttccccagctgagcgccattgctctgggagtggccacagcccacggaagcacactggctggcgtgaacgtgggcgagcagtaccagcagctgagagaggccgccacagaggccgagaaacagctgcagcagtacgccgagagcagagagctggatcacctgggcctggacgaccaggaaaagaaaatcctgatgaacttccaccagaagaagaacgagatcagcttccagcagaccaacgccatggtcaccctgcggaaagagcggctggccaaactgaccgaggccatcacagccgcctccctgcctaagacaagcggccactacgacgacgatgacgacatccccttccctggccccatcaatgacgacgacaaccctggccaccaggacgacgaccctaccgacagccaggacaccaccatccccgacgtggtggtggaccctgatgatggcggctacggcgagtaccagagctacagcgagaacggcatgagcgcccctgacgacctggtgctgttcgacctggacgaggacgatgaggataccaagcccgtgcccaacagaagcaccaagggcggccagcagaagaacagccagaagggccagcacaccgagggcagacagacccagagcacccccacccagaatgtgaccggccccagaagaaccatccaccacgcaagcgcccctctgaccgacaacgacagaagaaacgagcccagcggcagcaccagccccaggatgctgacccccattaacgaggaagccgaccccctggacgatgccgacgacgagacatctagcctgccccccctggaatccgatgacgaggaacaggacagggacggcaccagcaacagaacccctacagtggcccctcccgcccctgtgtacagagatcactccgagaagaaagagctgccccaggacgagcagcaggaccaggatcacatccaggaagcccggaaccaggacagcgacaacacccagcccgagcacagcttcgaggaaatgtaccggcacatcctgagatcccagggccccttcgatgccgtgctgtactatcacatgatgaaggacgagcccgtggtgttcagcacctccgacggcaaagagtacacctaccccgacagcctggaagaagagtaccccccttggctgacagagaaagaagccatgaacgacgagaaccgcttcgtgaccctggatggccagcagttttactggcccgtgatgaaccaccggaacaagttcatggccattctgcagcaccaccagtga

SEQ ID NO: 97 - RNA, 2031 nt, synthetic construct
  misc_feature 1..2031: note = BDBV GP, Uganda 2007,optimized mRNA Sequence
  source 1..2031: mol_type = other RNA
atggtcaccagcggcatcctgcagctgcccagagagcggttcagaaagaccagcttcttcgtgtgggtcatcatcctgttccacaaggtgttccccatccccctgggcgtggtgcacaacaacacactgcaggtgtccgacatcgacaagctcgtgtgccgggacaagctgagcagcaccagccagctgaagtccgtgggcctgaacctggaaggcaatggcgtggccaccgatgtgcctaccgccaccaagagatggggcttcagagctggcgtgccccccaaggtcgtgaattatgaggccggcgagtgggccgagaactgctacaacctggacatcaagaaggccgacggcagcgagtgtctgcctgaggctcctgaaggcgtgcggggcttccccagatgcagatacgtgcacaaagtgtccggcaccggcccttgccctgagggctacgcctttcacaaagagggcgcctttttcctgtacgaccggctggcctccaccatcatctacagaagcaccaccttcagcgagggggtggtggccttcctgatcctgcccgagacaaagaaggacttcttccagagccccccactgcacgagcccgccaacatgaccacagaccccagcagctactaccacaccgtgaccctgaactacgtggccgacaacttcgggaccaatatgaccaacttcctgtttcaagtggaccacctgacctacgtgcagctggaaccccggttcaccccccagtttctggtgcagctgaacgagacaatctacaccaacggcaggcggagcaacaccaccggcaccctgatctggaaagtgaaccccaccgtggacaccggcgtgggagaatgggccttctgggagaacaagaagaacttcaccaagaccctgagcagcgaggaactgagcgtgatcttcgtgcccagagcccaggaccccggcagcaaccagaaaacaaaagtgacccctaccagcttcgccaacaaccagaccagcaagaaccacgaggacctggtgcccgaggatcctgcctctgtggtgcaagtgcgggacctgcagcgcgagaataccgtgcctaccccccctcccgatactgtgcccaccacactgatccccgacaccatggaagaacagaccaccagccactacgagccccctaacatcagccggaaccaccaggaacggaacaacaccgcccaccccgagacactggccaacaatccccccgacaataccacccccagcacccctccacaggacggcgagagaacaagcagccacaccacccctagccccagacctgtgccaaccagcaccatccaccccaccaccagagagacacacatccctaccaccatgaccacctcccacgacaccgacagcaaccggcccaaccccatcgacatcagcgagagcacagagcctggccccctgaccaacaccacaagaggcgccgctaatctgctgaccggctccagacggaccagaagagagatcaccctgcggacccaggccaagtgcaaccccaacctgcactactggaccacccaggatgagggcgctgctatcggactggcctggatcccttactttggccctgccgccgagggcatctacacagagggcatcatgcacaaccagaacggcctgatctgcggcctgaggcagctggccaatgagacaacacaggctctgcagctgttcctgagagccaccaccgagctgcggaccttcagcatcctgaacagaaaggccatcgacttcctgctgcagcgctggggcggcacctgtcacattctgggccctgactgctgcatcgagccccacgactggaccaagaacatcaccgacaagatcgaccagatcatccacgacttcatcgataagcccctgcccgaccagaccgacaacgacaattggtggacaggctggcggcagtgggtgccagccggaatcggaatcacaggcgtgatcattgccgtgatcgctctgctgtgcatctgcaagtttctgctgtga

SEQ ID NO: 98 - RNA, 2031 nt, synthetic construct
  misc_feature 1..2031: note = SUDV GP, Gulu, Uganda 2007, optimized mRNA Sequence
  source 1..2031: mol_type = other RNA
atgggcggactgtctctgctgcagctgccccgggacaagttccggaagtccagcttcttcgtgtgggtcatcatcctgttccagaaagccttcagcatgcccctgggcgtcgtgaccaacagcaccctggaagtgaccgagatcgatcagctcgtgtgcaaggaccacctggccagcaccgaccagctgaagtccgtgggcctgaacctggaaggcagcggcgtgtccaccgatatccctagcgccaccaagagatggggcttcagatccggcgtgccccccaaggtggtgtcttatgaagccggcgagtgggccgagaactgctacaacctggaaatcaagaagcccgacggcagcgagtgcctgccccctcctcctgatggcgtgcggggcttccctagatgcagatacgtgcacaaggcccagggcaccggaccttgccctggcgattacgccttccacaaggacggcgcctttttcctgtacgaccggctggcctccaccgtgatctaccggggagtgaatttcgccgagggcgtgatcgccttcctgatcctggccaagcccaaagagacattcctgcagagcccccccatccgcgaggccgtgaactacaccgagaacaccagcagctactacgccaccagctacctggaatacgagatcgagaacttcggcgcccagcacagcaccaccctgttcaagatcgacaacaacaccttcgtgcggctggacagaccccacaccccccagtttctgttccagctgaacgacaccatccatctgcatcagcagctgagcaacaccaccggcagactgatctggaccctggacgccaacatcaacgccgatattggagagtgggccttctgggagaacaagaagaacctgagcgagcagctgcggggcgaggaactgtcttttgaggccctgagcctgaacgagacagaggacgatgatgccgccagcagccggatcaccaagggcagaatcagcgaccgggccacccggaagtacagcgacctggtgcccaagaacagccccggcatggtgcctctgcacatccctgagggcgagacaaccctgcccagccagaatagcacagaggggcgcagagtgggcgtgaacacccaggaaaccatcaccgagacagccgccaccatcatcggcaccaacggcaaccacatgcagatcagcaccatcggcatccggcccagcagcagccagatccctagcagctctcctaccaccgcccctagccctgaagcccagacccctaccacacacacaagcggcccaagcgtgatggccaccgaggaacctacaacccctcctggctctagccctggccctacaacagaggcccctacactgaccacccccgagaacatcaccaccgccgtgaaaaccgtgctgccccaggaaagcaccagcaacggcctgatcaccagcaccgtgacaggcatcctgggcagcctgggcctgcggaagagaagcagaaggcagaccaacaccaaggccaccggcaagtgcaaccccaacctgcactactggaccgcccaggaacagcataacgccgctggaatcgcctggatcccctactttggacctggcgccgagggaatctacacagagggcctgatgcacaaccagaacgccctcgtgtgcggcctgagacagctggccaacgagacaacacaggccctgcagctgtttctgcgggccaccaccgagctgcggacctacaccatcctgaacagaaaggccatcgactttctgctgagaagatggggcggcacctgtcggatcctgggccctgattgctgcatcgagccccacgactggaccaagaatatcaccgacaagatcaaccagatcatccacgacttcatcgacaaccccctgcccaatcaggacaacgacgacaactggtggaccggatggcggcagtggattcctgccggcatcggaatcaccggcatcattatcgccatcattgccctgctgtgcgtgtgcaaactgctgtgttga

SEQ ID NO: 99 - RNA, 2031 nt, synthetic construct
  misc_feature 1..2031: note = TAFV GP, Cote dIvoire 1994, optimized mRNA Sequence
  source 1..2031: mol_type = other RNA
atgggcgcctctggaatcctgcagctgcccagagagcggttcagaaagaccagcttcttcgtgtgggtcatcatcctgttccacaaggtgttcagcatccccctgggcgtggtgcacaacaacaccctgcaggtgtccgacatcgacaagttcgtgtgccgggacaagctgagcagcaccagccagctgaagtccgtgggcctgaacctggaaggcaatggcgtggccaccgatgtgcctaccgccaccaagagatggggcttcagagctggcgtgccccccaaggtcgtgaattgtgaagccggcgagtgggccgagaactgctacaacctggccatcaagaaggtggacggcagcgagtgtctgcccgaagctcctgaaggcgtgcgggacttcccccggtgtagatacgtgcacaaagtgtccggcaccggcccttgtcctggcggactggcctttcacaaagagggcgcctttttcctgtacgaccggctggcctccaccatcatctaccggggcaccacatttgccgagggcgtgatcgccttcctgatcctgcccaaggcccggaaggacttcttccagagccctccactgcacgagcccgccaacatgaccaccgaccccagcagctactaccacaccaccaccatcaattacgtggtggacaacttcggcaccaacaccaccgagtttctgtttcaagtggaccacctgacctacgtgcagctggaagcccggttcaccccccagtttctggtgctgctgaacgagacaatctacagcgacaaccggcggagcaataccaccggcaagctgatctggaagatcaaccccaccgtggacacctctatgggagagtgggccttctgggagaacaagaagaacttcaccaagaccctgagcagcgaggaactgagcttcgtgcccgtgcccgagacacagaaccaggtgctggataccaccgccaccgtgtccccacctatcagcgcccataatcacgccgccgaggaccacaaagaactggtgtccgaggacagcacccccgtggtgcagatgcagaacatcaagggcaaggacaccatgcccaccaccgtgaccggcgtgccaaccacaacccctagccccttccctatcaacgcccggaacaccgaccacaccaagagcttcatcggcctggaaggaccccaggaagatcactccaccacccagcctgccaagaccacaagccagcccaccaatagcaccgagagcaccaccctgaaccccaccagcgagcctagctctagaggcacaggccctagcagccctaccgtgcccaataccacagagagccacgccgagctgggcaagaccacccctacaacactgcccgaacagcacacagccgccagcgccattcctagagccgtgcaccctgatgagctgagcggccctggcttcctgaccaataccatccggggcgtgaccaacctgctgaccggctctagaagaaagcggcgggacgtgacccccaacacccagcccaagtgcaaccccaacctgcactactggaccgccctggatgagggcgctgctatcggactggcttggatcccctactttggccctgccgccgaaggcatctacacagagggcatcatggaaaaccagaacggcctgatctgcggcctgcggcagctggccaatgagacaacacaggctctgcagctgttcctgcgggccaccacagagctgcggaccttctccatcctgaacagaaaggccatcgacttcctgctgcagcgctggggcggcacctgtcacattctgggccctgactgctgcatcgagccccaggactggaccaagaacatcaccgacaagatcgaccagatcatccacgacttcgtggacaacaacctgcccaaccagaatgacggcagcaactggtggacaggctggaagcagtgggtgccagccggcatcggaatcaccggcgtgatcattgccattatcgccctgctgtgtatctgcaagttcatgctgtga

SEQ ID NO: 100 - RNA, 981 nt, synthetic construct
  misc_feature 1..981: note = BDBV VP40, Uganda 2007, optimized mRNA Sequence
  source 1..981: mol_type = other RNA
atgcggagagccatcctgcctacagccccccctgagtacatcgaggccgtgtaccccatgcggaccgtgtccaccagcatcaacagcacagccagcggccccaacttccctgcccccgatgtgatgatgagcgacacccccagcaacagcctgaggcctatcgccgacgacaacatcgaccaccccagccacacccctaccagcgtgtccagcgccttcatcctggaagccatggtcaacgtgatctccggccccaaggtgctgatgaagcagatccccatctggctgcctctgggcgtggccgaccagaaaacctacagcttcgacagcaccaccgccgccatcatgctggcctcttacaccatcacccacttcggcaagaccagcaaccccctcgtgcggatcaacagactgggccctggcatccccgaccaccctctgagactgctgagaatcggcaaccaggcctttctgcaggaatttgtgctgccccccgtgcagctgccccagtacttcacctttgacctgaccgccctgaagctgatcacccagcctctgcctgccgccacctggaccgatgatacacctacaggccccaccggcatcctgaggcccggaatcagcttccaccccaagctgaggcccatcctgctgcctggcaagacaggcaagagaggcagcagcagcgatctgaccagccccgacaagatccaggccatcatgaattttctgcaggacctgaagctggtgcccatcgaccccgccaagaacatcatgggcatcgaggtgcccgagctgctggtgcacagactgaccggcaagaagatcaccaccaagaacggccagcccatcatccctattctgctgcccaagtacatcggcatggaccccatcagccagggcgacctgaccatggtcatcacccaggactgcgatacctgccactcccctgcttctctgcccccagtgtccgagaagtga

SEQ ID NO: 101 - RNA, 981 nt, synthetic construct
  misc_feature 1..981: note = SUDV VP40, Gulu, Uganda 2000, optimized mRNA Sequence
  source 1..981: mol_type = other RNA
atgcggagagtgaccgtgcctacagcccctcctgcctatgccgacatcggctaccccatgagcatgctgcccatcaagagcagcagagccgtgtccggcatccagcagaaacaggaagtgctgcccggcatggacacccccagcaactctatgaggcccgtggccgacgacaacatcgaccacaccagccacacccccaatggcgtggccagcgcctttatcctggaagccaccgtgaacgtgatcagcggccccaaggtgctgatgaagcagatccccatctggctgcccctgggaatcgccgaccagaaaacctacagcttcgacagcaccaccgccgccatcatgctggcctcttacaccatcacccacttcggcaaggccaacaaccccctggtgcgcgtgaacagactgggccagggaatccccgatcaccccctgagactgctgcggatgggcaaccaggcctttctgcaggaatttgtgctgccccccgtgcagctgccccagtacttcacctttgacctgaccgccctgaagctcgtgacccagcctctgcctgccgccacctggacagatgagacacccagcaatctgtctggcgccctgaggcctggcctgagctttcaccctaagctgcggcctgtgctgctgcctggcaagaccggcaagaaaggccacgtgtccgacctgacagcccccgacaagatccagaccatcgtgaacctgatgcaggacttcaagatcgtgcccatcgaccccgccaagagcatcatcggcatcgaggtgccagagctgctggtgcacaagctgacaggcaagaagatgagccagaagaacggccagcccatcatccccgtgctgctgccaaagtacatcggcctggaccccatcagccctggcgacctgaccatggtcatcacccccgactacgacgactgccacagccctgccagctgctcctacctgagcgagaagtga

SEQ ID NO: 102 - RNA, 981 nt, synthetic construct
  misc_feature 1..981: note = TAFV VP40, Cote dIvoire 1994, optimized mRNA Sequence
  source 1..981: mol_type = other RNA
atgcggagaatcatcctgcctaccgcccctcccgagtacatggaagccgtgtaccccatgcggaccatgaactctggcgccgacaataccgccagcggccccaattacaccaccaccggcgtgatgaccaacgacacccccagcaacagcctgaggcccgtggccgacgacaacatcgatcaccctagccacacccccaacagcgtggccagcgcctttatcctggaagccatggtcaacgtgatctccggccccaaggtgctgatgaagcagatccccatctggctgcccctgggcgtgtccgaccagaaaacctacagcttcgacagcaccaccgccgccatcatgctggcctcttacaccatcacccacttcggcaagaccagcaaccccctcgtgcggatcaacagactgggccctggcatccctgaccaccccctgagactgctgagaatcggcaaccaggcctttctgcaggaatttgtgctgccccccgtgcagctgccccagtacttcacctttgacctgaccgccctgaagctgatcacccagcctctgcctgccgccacctggaccgatgagactcctgccgtgtctaccggcacactgaggcctggcatcagcttccaccccaagctgaggcccatcctgctgcctggcagagccggcaagaagggcagcaatagcgatctgaccagccccgacaagatccaggccatcatgaattttctgcaggacctgaagatcgtgcccatcgaccccaccaagaacatcatgggcatcgaggtgcccgagctgctggtgcacagactgaccggcaaaaagaccaccacaaagaacggccagcccatcatccctattctgctgcccaagtacatcggcctggaccccctgtctcagggcgacctgaccatggtcatcacccaggactgcgacagctgccactctcctgctagcctgccccctgtgaacgagaagtga

SEQ ID NO: 103 - RNA, 2088 nt, synthetic construct
  misc_feature 1..2088: note = MARV NP, Angola 2005, optimized mRNA Sequence
  source 1..2088: mol_type = other RNA
atggacctgcacagcctgctggaactgggcaccaagcctacagcccctcacgtgcggaacaagaaagtgatcctgttcgacaccaaccaccaggtgtccatctgcaaccagatcatcgacgccatcaacagcggcatcgacctgggcgatctgctggaaggcggactgctgaccctgtgcgtggaacactactacaacagcgacaaggacaagttcaacaccagccctatcgccaagtacctgcgggacgccggctacgagttcgacgtgatcaagaacgccgacgccacccggtttctggacgtgatccccaacgagccccactactccccactgatcctggccctgaaaaccctggaaagcaccgagagccagcggggcagaatcggcctgttcctgagcttctgcagcctgtttctgcccaagctggtcgtgggcgacagagcctctatcgagaaggccctgagacaagtgaccgtgcaccaggaacagggcatcgtgacctaccccaaccactggctgaccaccggccacatgaaagtgattttcggcatcctgcggagcagcttcatcctgaagttcgtgctgatccaccagggcgtgaacctcgtgacaggccacgacgcctacgacagcatcatcagcaacagcgtgggccagacccggttcagcggcctgctgatcgtgaaaaccgtgctggagttcatcctgcagaaaaccgacagcggcgtgaccctgcatcctctcgtgcggacctccaaagtgaagaacgaggtggccagcttcaagcaggccctgtccaacctggccagacacggcgagtatgcccccttcgccagagtgctgaacctgagcggcatcaacaacctggaacacggcctgtacccccagctgagcgccattgctctgggagtggccacagcccacggatctacactggctggcgtgaacgtgggcgagcagtaccagcagctgagagaggctgcccacgacgctgaagtgaagctgcagcggagacacgaacaccaggaaatccaggccattgccgaggacgacgaggaacggaagatcctggaacagttccatctgcagaaaacagagatcacccacagccagaccctggccgtgctgagccagaagagagagaaactggccaggctggccgccgagatcgagaacaacatcgtggaagatcaggggttcaagcagtcccagaacagagtgtcccagagcttcctgaacgaccccacccccgtggaagtgacagtgcaggccagacctatcaaccggcctaccgctctgccccctccagtggactctaagatcgagcacgagagcaccgaggacagcagcagctccagcagcttcgtggacctgaacgatcccttcgccctgctgaacgaggacgaggacaccctggacgacagcgtgatgatccccagcaccaccagcagagagttccagggcatccccgagccccctagacagagccaggacatcgacaacagccagggcaagcaggaagatgagagcaccaacctgattaagaagcccttcctgcgctaccaggaactgccccccgtgcaggaagatgacgagagcgagtacaccaccgacagccaggaatccatcgaccagcccggcagcgacaatgagcagggggtggacctgcctccccctccactgtatgcccaggaaaagcggcaggaccccatccagcatcctgccgtgtcaagccaggacccctttggctctatcggcgacgtgaacggcgacattctggaacccatccggtcccccagctctccaagtgcccctcaggaagatacccgggccagagaggcctacgagctgagccccgacttcaccaactacgaggacaaccagcagaactggccccagcgggtcgtgaccaagaagggcagaaccttcctgtaccctaacgacctgctgcagaccaacccccccgagagcctgattaccgcactggtggaagagtaccagaaccccgtgtccgccaaagagctgcaggccgactggcccgacatgagcttcgacgagagaaggcacgtggccatgaacctgtga

SEQ ID NO: 104 - RNA, 2220 nt, synthetic construct
  misc_feature 1..2220: note = BDBV NP, Uganda 2007, optimized mRNA Sequence
  source 1..2220: mol_type = other RNA
atggaccccagacccatccggacctggatgatgcacaacaccagcgaggtggaagccgactaccacaagatcctgacagccggcctgagcgtgcagcagggaattgtgcggcagcggatcatccccgtgtaccagatcagcaacctggaagaagtgtgccagctgatcattcaggccttcgaggccggcgtggacttccaggatagcgccgatagcttcctgctgatgctgtgcctgcaccacgcctaccagggcgactacaagcagttcctggaaagcaacgccgtgaagtacctggaaggacacggcttcagattcgagatgaagaaaaaagaaggcgtgaagcggctggaagaactgctgcctgccgccagcagcggcaagaacatcaagagaaccctggccgccatgcccgaggaagagacaacagaggccaacgccggccagttcctgagcttcgccagcctgttcctgcccaagctggtcgtgggagagaaggcctgcctggaaaaggtgcagcggcagatccaggtgcacgccgagcagggcctgatccagtaccctacaagctggcagagcgtgggccacatgatggtcatcttccggctgatgaggaccaacttcctgatcaagtttctgctgatccaccagggcatgcacatggtggccggacacgacgccaacgatgccgtgatcgccaattctgtggcccaggccagattcagcggcctgctgatcgtgaaaaccgtgctggaccacatcctgcagaaaaccgagcacggcgtgcggctgcatccactggccagaaccgccaaagtgaagaacgaggtgtccagcttcaaggccgccctggcctctctggctcagcacggcgaatatgcccccttcgccagactgctgaacctgagcggcgtgaacaatctggaacacggcctgtttccccagctgagcgccattgctctgggagtggccacagcccacggaagcacactggctggcgtgaacgtgggcgagcagtaccagcagctgagagaggccgccacagaggccgagaagcagctgcagaagtacgccgagagcagagagctggatcacctgggcctggacgaccaggaaaagaagattctgaaggacttccaccagaagaagaacgaaatcagcttccagcagaccaccgccatggtcaccctgcggaaagagagactggccaagctgaccgaggccatcaccagcaccagcatcctgaaaaccggcagaagatacgacgacgacaacgacatccccttccctggccccatcaacgacaatgagaacagcggccagaacgacgatgaccccaccgacagccaggacaccaccatccccgacgtgatcatcgaccccaacgacggcggctacaacaactacagcgactacgccaatgacgccgccagcgcccctgatgacctggtgctgttcgatctggaagatgaggacgacgccgacaatcccgcccagaacacccccgagaagaatgacagacccgccaccaccaagctgcggaacggccaggatcaggacggcaatcagggcgagacagccagccctagagtggcccccaaccagtacagagacaagcccatgccccaggtgcaggacagaagcgagaaccacgatcagaccctgcagacccagagcagggtgctgacccccatcagcgaagaggccgaccccagcgaccacaacgatggcgacaacgagagcatcccccccctggaatccgacgatgagggcagcaccgataccacagccgccgagacaaaacctgccactgctcctcccgcccctgtgtacagatccatcagcgtggacgacagcgtgcccagcgagaacatccccgcccagtccaaccagaccaacaacgaggacaacgtgcggaacaacgcccagagcgagcagtctatcgccgagatgtaccagcacattctgaaaacccagggccccttcgacgccatcctgtactatcacatgatgaaggaagaacccatcatcttcagcacctccgacggcaaagagtacacctaccccgactccctggaagatgaataccccccctggctgagcgagaaagaagccatgaacgaggataaccggttcatcaccatggacggccagcagttttactggcccgtgatgaatcaccggaacaagttcatggccattctgcagcaccaccggtga

SEQ ID NO: 105 - RNA, 2217 nt, synthetic construct
  misc_feature 1..2217: note = SUDV NP, Gulu, Uganda 2000,optimized mRNA Sequence
  source 1..2217: mol_type = other RNA
atggacaagagagtgcggggctcttgggccctgggaggacagtctgaagtggacctggactaccacaagatcctgacagccggcctgagcgtgcagcagggaattgtgcggcagcgcgtgatccccgtgtacgtggtgtctgacctggaaggcatctgccagcacatcatccaggccttcgaagccggcgtggacttccaggacaacgccgacagcttcctgctgctgctgtgtctgcaccacgcctaccagggcgaccaccggctgtttctgaagtccgatgccgtgcagtatctggaaggccacggcttcagattcgaagtgcgcgagaaagaaaacgtgcaccggctggacgagctgctgcccaatgtgaccggcggcaagaacctgagaagaaccctggccgccatgcccgaggaagagacaacagaggccaacgccggccagttcctgagcttcgccagcctgttcctgcccaagctggtcgtgggagagaaggcctgcctggaaaaggtgcagcggcagatccaggtgcacgccgagcagggcctgatccagtaccctacaagctggcagagcgtgggccacatgatggtcatcttccggctgatgcggaccaacttcctgatcaagttcctgctgatccaccagggcatgcacatggtggccggacacgacgccaacgacaccgtgatcagcaactctgtggcccaggccagattcagcggactgctgatcgtgaaaaccgtgctggaccacatcctgcagaaaaccgacctgggcgtgcggctgcatccactggccagaaccgccaaagtgaagaacgaggtgtccagcttcaaggccgccctgggctctctggccaagcacggcgaatatgcccccttcgccagactgctgaacctgagcggcgtgaacaacctggaacacggcctgtacccccagctgagcgccattgctctgggagtggccacagcccacggaagcacactggctggcgtgaacgtgggcgagcagtaccagcagctgagagaggccgccacagaggccgagaaacagctgcagcagtacgccgaaaccagagagctggacaacctgggcctggacgaacaggaaaagaaaatcctgatgagcttccaccagaagaagaacgaaatcagcttccagcagaccaacgccatggtcaccctgcggaaagagcggctggccaaactgaccgaggccatcaccaccgccagcaagatcaaagtgggcgacagataccccgacgacaacgatatccccttccctggccccatctacgacgagacacaccccaaccccagcgacgacaatcccgacgactcccgggataccacaatccctggcggcgtggtggacccctacgacgacgagagcaacaactaccccgactacgaggacagcgccgagggcacaacaggcgacctggacctgttcaacctggacgacgatgacgacgacagccagcctggccctcctgatagaggccagagcaaagaaagagccgccagaacccacggcctgcaggaccctacactggacggcgccaagaaggtgccagagctgacacctggcagccaccagccaggcaacctgcacatcacaaagcccggcagcaacaccaaccagccccagggcaatatgagcagcaccctgcagagcatgacccccatccaggaagagagcgagcccgacgaccagaaagacgatgatgatgagagcctgaccagcctggacagcgagggcgacgaggatgtggaatccgtgtctggcgagaacaaccccaccgtggctcctccagcccctgtgtacaaggacacaggcgtggacaccaatcagcagaacggccccagcaacgccgtggatggacagggctctgagtctgaggccctgcccatcaaccctgagaagggcagcgccctggaagaaacctactaccatctgctgaaaacacagggccccttcgaggccattaactactaccacctgatgtccgacgagcctatcgccttcagcaccgagagcggcaaagagtacatcttccccgacagcctggaagaggcctaccccccttggctgtccgagaaagaggccctggaaaaagaaaaccgctacctcgtgatcgacggccagcagttcctgtggcccgtgatgtccctgcaggataagttcctggccgtgctgcagcatgactga

SEQ ID NO: 106 - RNA, 2220 nt, synthetic construct
  misc_feature 1..2220: note = TAFV NP, Cote dIvoire 1994,optimized mRNA Sequence
  source 1..2220: mol_type = other RNA
atggaaagcagagcccacaaggcctggatgacccacaccgccagcggcttcgagacagactaccacaagatcctgacagccggcctgagcgtgcagcagggaattgtgcggcagcgcgtgatccaggtgcaccaagtgaccaacctggaagagatctgccagctgatcattcaggccttcgaggccggcgtggacttccaggaaagcgccgatagcttcctgctgatgctgtgcctgcaccacgcctaccagggcgactacaagcagttcctggaaagcaacgccgtgaagtacctggaaggacacggcttcagattcgaagtgcggaagaaagaaggcgtgaagcggctggaagaactgctgcctgccgccagcagcggcaagagcattagaagaaccctggccgccatgcccgaggaagagacaacagaggccaacgccggccagttcctgagcttcgccagcctgttcctgcccaagctggtcgtgggagagaaggcctgcctggaaaaggtgcagcggcagattcaggtgcacagcgagcagggcctgatccagtaccctacagcctggcagagcgtgggccacatgatggtcatcttccggctgatgaggaccaacttcctgatcaagtttctgctgatccaccagggcatgcacatggtggccggacacgacgccaacgatgccgtgatcgccaattctgtggcccaggccagattcagcggcctgctgatcgtgaaaaccgtgctggaccacatcctgcagaaaaccgagcacggcgtgcggctgcatccactggccagaaccgccaaagtgaagaacgaagtgaacagcttcaaggccgccctgagcagcctggcccagcatggcgaatatgcccccttcgccagactgctgaacctgagcggcgtgaacaatctggaacacggcctgtttccccagctgagcgccattgctctgggagtggccacagcccacggaagcacactggctggcgtgaacgtgggcgagcagtaccagcagctgagagaggccgccacagaggccgagaagcagctgcagaagtacgccgagagcagagagctggatcacctgggcctggacgaccaggaaaagaagattctgaaggacttccaccagaagaagaacgagatcagcttccagcagaccaccgccatggtcaccctgcggaaagagagactggccaagctgaccgaggccatcaccagcaccagcctgctgaaaacaggcaagcagtacgacgacgacaacgacatccccttccctggccccatcaacgacaatgagaactccgagcagcaggacgacgatcccaccgacagccaggacaccaccatccccgacatcatcgtggaccccgacgacggccggtacaacaactacggcgactaccccagcgagacagccaacgcccctgaggacctggtgctgttcgatctggaagatggcgacgaggacgaccacagacccagcagcagctccgagaacaacaacaagcacagcctgaccggcaccgactccaacaagaccagcaactggaaccggaaccccaccaacatgcccaagaaggacagcacccagaacaacgacaaccctgcccagcgggcccaggaatacgccagggacaacatccaggatacccccacccctcacagagccctgacccccatctctgaggaaaccggcagcaacggccacaacgaggatgacatcgacagcatcccccccctggaatccgacgaggaaaacaacaccgagacaaccatcaccaccacaaagaacaccaccgcccctcccgcccctgtgtacagaagcaacagcgagaaagagcccctgcctcaggaaaagtcccagaaacagcccaaccaggtgtccggcagcgagaacaccgataacaagccccacagcgaacagtccgtggaagaaatgtaccggcacattctgcagacccagggccccttcgacgccatcctgtactactacatgatgaccgaggaacccatcgtgttcagcacctccgacggcaaagaatacgtgtaccccgacagcctggaaggcgagcaccctccatggctgtctgagaaagaagccctgaacgaggacaaccggttcatcaccatggatgaccagcagttttactggcccgtgatgaatcaccggaacaagttcatggctatcctgcagcaccacaagtga

SEQ ID NO: 107 - RNA, 2031 nt, synthetic construct
  misc_feature 1..2031: note = EBOV GP, Mayinga, Zaire 1976, optimized mRNA Sequence
  source 1..2031: mol_type = other RNA
atgggcgtgaccggcatcctgcagctgcccagagacagattcaagagaaccagcttcttcctgtgggtgatcatcctgttccagagaaccttcagcatccccctgggcgtgatccacaacagcaccctgcaggtgagcgacgtggacaagctggtgtgcagagacaagctgagcagcaccaaccagctgagaagcgtgggcctgaacctggagggcaacggcgtggccaccgacgtgcccagcgccaccaagagatggggcttcagaagcggcgtgccccccaaggtggtgaactacgaggccggcgagtgggccgagaactgctacaacctggagatcaagaagcccgacggcagcgagtgcctgcccgccgcccccgacggcatcagaggcttccccagatgcagatacgtgcacaaggtgagcggcaccggcccctgcgccggcgacttcgccttccacaaggagggcgccttcttcctgtacgacagactggccagcaccgtgatctacagaggcaccaccttcgccgagggcgtggtggccttcctgatcctgccccaggccaagaaggacttcttcagcagccaccccctgagagagcccgtgaacgccaccgaggaccccagcagcggctactacagcaccaccatcagataccaggccaccggcttcggcaccaacgagaccgagtacctgttcgaggtggacaacctgacctacgtgcagctggagagcagattcaccccccagttcctgctgcagctgaacgagaccatctacaccagcggcaagagaagcaacaccaccggcaagctgatctggaaggtgaaccccgagatcgacaccaccatcggcgagtgggccttctgggagaccaagaagaacctgaccagaaagatcagaagcgaggagctgagcttcaccgtggtgagcaacggcgccaagaacatcagcggccagagccccgccagaaccagcagcgaccccggcaccaacaccaccaccgaggaccacaagatcatggccagcgagaacagcagcgccatggtgcaggtgcacagccagggcagagaggccgccgtgagccacctgaccaccctggccaccatcagcaccagcccccagagcctgaccaccaagcccggccccgacaacagcacccacaacacccccgtgtacaagctggacatcagcgaggccacccaggtggagcagcaccacagaagaaccgacaacgacagcaccgccagcgacacccccagcgccaccaccgccgccggcccccccaaggccgagaacaccaacaccagcaagagcaccgacttcctggaccccgccaccaccaccagcccccagaaccacagcgagaccgccggcaacaacaacacccaccaccaggacaccggcgaggagagcgccagcagcggcaagctgggcctgatcaccaacaccatcgccggcgtggccggcctgatcaccggcggcagaagaaccagaagagaggccatcgtgaacgcccagcccaagtgcaaccccaacctgcactactggaccacccaggacgagggcgccgccatcggcctggcctggatcccctacttcggccccgccgccgagggcatctacatcgagggcctgatgcacaaccaggacggcctgatctgcggcctgagacagctggccaacgagaccacccaggccctgcagctgttcctgagagccaccaccgagctgagaaccttcagcatcctgaacagaaaggccatcgacttcctgctgcagagatggggcggcacctgccacatcctgggccccgactgctgcatcgagccccacgactggaccaagaacatcaccgacaagatcgaccagatcatccacgacttcgtggacaagaccctgcccgaccagggcgacaacgacaactggtggaccggctggagacagtggatccccgccggcatcggcgtgaccggcgtgatcatcgccgtgatcgccctgttctgcatctgcaagttcgtgttctga

SEQ ID NO: 108 - RNA, 2031 nt, synthetic construct
  misc_feature 1..2031: note = EBOV GP, Sierra Leone 2014, optimized mRNA Sequence
  source 1..2031: mol_type = other RNA
atgggcgtgaccggcatcctgcagctgcccagagacagattcaagagaaccagcttcttcctgtgggtgatcatcctgttccagagaaccttcagcatccccctgggcgtgatccacaacagcaccctgcaggtgagcgacgtggacaagctggtgtgcagagacaagctgagcagcaccaaccagctgagaagcgtgggcctgaacctggagggcaacggcgtggccaccgacgtgcccagcgtgaccaagagatggggcttcagaagcggcgtgccccccaaggtggtgaactacgaggccggcgagtgggccgagaactgctacaacctggagatcaagaagcccgacggcagcgagtgcctgcccgccgcccccgacggcatcagaggcttccccagatgcagatacgtgcacaaggtgagcggcaccggcccctgcgccggcgacttcgccttccacaaggagggcgccttcttcctgtacgacagactggccagcaccgtgatctacagaggcaccaccttcgccgagggcgtggtggccttcctgatcctgccccaggccaagaaggacttcttcagcagccaccccctgagagagcccgtgaacgccaccgaggaccccagcagcggctactacagcaccaccatcagataccaggccaccggcttcggcaccaacgagaccgagtacctgttcgaggtggacaacctgacctacgtgcagctggagagcagattcaccccccagttcctgctgcagctgaacgagaccatctacgccagcggcaagagaagcaacaccaccggcaagctgatctggaaggtgaaccccgagatcgacaccaccatcggcgagtgggccttctgggagaccaagaagaacctgaccagaaagatcagaagcgaggagctgagcttcaccgccgtgagcaacggccccaagaacatcagcggccagagccccgccagaaccagcagcgaccccgagaccaacaccaccaacgaggaccacaagatcatggccagcgagaacagcagcgccatggtgcaggtgcacagccagggcagaaaggccgccgtgagccacctgaccaccctggccaccatcagcaccagcccccagccccccaccaccaagaccggccccgacaacagcacccacaacacccccgtgtacaagctggacatcagcgaggccacccaggtgggccagcaccacagaagagccgacaacgacagcaccgccagcgacaccccccccgccaccaccgccgccggccccctgaaggccgagaacaccaacaccagcaagagcgccgacagcctggacctggccaccaccaccagcccccagaactacagcgagaccgccggcaacaacaacacccaccaccaggacaccggcgaggagagcgccagcagcggcaagctgggcctgatcaccaacaccatcgccggcgtggccggcctgatcaccggcggcagaagaaccagaagagaggtgatcgtgaacgcccagcccaagtgcaaccccaacctgcactactggaccacccaggacgagggcgccgccatcggcctggcctggatcccctacttcggccccgccgccgagggcatctacaccgagggcctgatgcacaaccaggacggcctgatctgcggcctgagacagctggccaacgagaccacccaggccctgcagctgttcctgagagccaccaccgagctgagaaccttcagcatcctgaacagaaaggccatcgacttcctgctgcagagatggggcggcacctgccacatcctgggccccgactgctgcatcgagccccacgactggaccaagaacatcaccgacaagatcgaccagatcatccacgacttcgtggacaagaccctgcccgaccagggcgacaacgacaactggtggaccggctggagacagtggatccccgccggcatcggcgtgaccggcgtgatcatcgccgtgatcgccctgttctgcatctgcaagttcgtgttctga

SEQ ID NO: 109 - RNA, 2046 nt, synthetic construct
  misc_feature 1..2046: note = MARV GP, Angola 2005, optimized mRNA Sequence
  source 1..2046: mol_type = other RNA
atgaagaccacctgcctgctgatcagcctgatcctgatccagggcgtgaagaccctgcccatcctggagatcgccagcaacatccagccccagaacgtggacagcgtgtgcagcggcaccctgcagaagaccgaggacgtgcacctgatgggcttcaccctgagcggccagaaggtggccgacagccccctggaggccagcaagagatgggccttcagagccggcgtgccccccaagaacgtggagtacaccgagggcgaggaggccaagacctgctacaacatcagcgtgaccgaccccagcggcaagagcctgctgctggacccccccaccaacatcagagactaccccaagtgcaagaccatccaccacatccagggccagaacccccacgcccagggcatcgccctgcacctgtggggcgccttcttcctgtacgacagaatcgccagcaccaccatgtacagaggcaaggtgttcaccgagggcaacatcgccgccatgatcgtgaacaagaccgtgcacaagatgatcttcagcagacagggccagggctacagacacatgaacctgaccagcaccaacaagtactggaccagcagcaacggcacccagaccaacgacaccggctgcttcggcaccctgcaggagtacaacagcaccaagaaccagacctgcgcccccagcaagaagcccctgcccctgcccaccgcccaccccgaggtgaagctgaccagcaccagcaccgacgccaccaagctgaacaccaccgaccccaacagcgacgacgaggacctgaccaccagcggcagcggcagcggcgagcaggagccctacaccaccagcgacgccgccaccaagcagggcctgagcagcaccatgccccccacccccagcccccagcccagcaccccccagcagggcggcaacaacaccaaccacagccagggcgtggtgaccgagcccggcaagaccaacaccaccgcccagcccagcatgcccccccacaacaccaccaccatcagcaccaacaacaccagcaagcacaacctgagcacccccagcgtgcccatccagaacgccaccaactacaacacccagagcaccgcccccgagaacgagcagaccagcgcccccagcaagaccaccctgctgcccaccgagaaccccaccaccgccaagagcaccaacagcaccaagagccccaccaccaccgtgcccaacaccaccaacaagtacagcaccagccccagccccacccccaacagcaccgcccagcacctggtgtacttcagaagaaagagaaacatcctgtggagagagggcgacatgttccccttcctggacggcctgatcaacgcccccatcgacttcgaccccgtgcccaacaccaagaccatcttcgacgagagcagcagcagcggcgccagcgccgaggaggaccagcacgccagccccaacatcagcctgaccctgagctacttccccaaggtgaacgagaacaccgcccacagcggcgagaacgagaacgactgcgacgccgagctgagaatctggagcgtgcaggaggacgacctggccgccggcctgagctggatccccttcttcggccccggcatcgagggcctgtacaccgccggcctgatcaagaaccagaacaacctggtgtgcagactgagaagactggccaaccagaccgccaagagcctggagctgctgctgagagtgaccaccgaggagagaaccttcagcctgatcaacagacacgccatcgacttcctgctggccagatggggcggcacctgcaaggtgctgggccccgactgctgcatcggcatcgaggacctgagcagaaacatcagcgagcagatcgaccagatcaagaaggacgagcagaaggagggcaccggctggggcctgggcggcaagtggtggaccagcgactggggcgtgctgaccaacctgggcatcctgctgctgctgagcatcgccgtgctgatcgccctgagctgcatctgcagaatcttcaccaagtacatcggctga

SEQ ID NO: 110 - RNA, 981 nt, synthetic construct
  misc_feature 1..981: note = EBOV VP40, Mayinga, Zaire 1976, optimized mRNA Sequence
  source 1..981: mol_type = other RNA
atgagaagagtgatcctgcccaccgccccccccgagtacatggaggccatctaccccgtgagaagcaacagcaccatcgccagaggcggcaacagcaacaccggcttcctgacccccgagagcgtgaacggcgacacccccagcaaccccctgagacccatcgccgacgacaccatcgaccacgccagccacacccccggcagcgtgagcagcgccttcatcctggaggccatggtgaacgtgatcagcggccccaaggtgctgatgaagcagatccccatctggctgcccctgggcgtggccgaccagaagacctacagcttcgacagcaccaccgccgccatcatgctggccagctacaccatcacccacttcggcaaggccaccaaccccctggtgagagtgaacagactgggccccggcatccccgaccaccccctgagactgctgagaatcggcaaccaggccttcctgcaggagttcgtgctgccccccgtgcagctgccccagtacttcaccttcgacctgaccgccctgaagctgatcacccagcccctgcccgccgccacctggaccgacgacacccccaccggcagcaacggcgccctgagacccggcatcagcttccaccccaagctgagacccatcctgctgcccaacaagagcggcaagaagggcaacagcgccgacctgaccagccccgagaagatccaggccatcatgaccagcctgcaggacttcaagatcgtgcccatcgaccccaccaagaacatcatgggcatcgaggtgcccgagaccctggtgcacaagctgaccggcaagaaggtgaccagcaagaacggccagcccatcatccccgtgctgctgcccaagtacatcggcctggaccccgtggcccccggcgacctgaccatggtgatcacccaggactgcgacacctgccacagccccgccagcctgcccgccgtgatcgagaagtga

SEQ ID NO: 111 - RNA, 981 nt, synthetic construct
  misc_feature 1..981: note = EBOV VP40, Sierra Leone 2014,optimized mRNA Sequence
  source 1..981: mol_type = other RNA
atgagaagagtgatcctgcccaccgccccccccgagtacatggaggccatctaccccgccagaagcaacagcaccatcgccagaggcggcaacagcaacaccggcttcctgacccccgagagcgtgaacggcgacacccccagcaaccccctgagacccatcgccgacgacaccatcgaccacgccagccacacccccggcagcgtgagcagcgccttcatcctggaggccatggtgaacgtgatcagcggccccaaggtgctgatgaagcagatccccatctggctgcccctgggcgtggccgaccagaagacctacagcttcgacagcaccaccgccgccatcatgctggccagctacaccatcacccacttcggcaaggccaccaaccccctggtgagagtgaacagactgggccccggcatccccgaccaccccctgagactgctgagaatcggcaaccaggccttcctgcaggagttcgtgctgccccccgtgcagctgccccagtacttcaccttcgacctgaccgccctgaagctgatcacccagcccctgcccgccgccacctggaccgacgacacccccaccggcagcaacggcgccctgagacccggcatcagcttccaccccaagctgagacccatcctgctgcccaacaagagcggcaagaagggcaacagcgccgacctgaccagccccgagaagatccaggccatcatgaccagcctgcaggacttcaagatcgtgcccatcgaccccaccaagaacatcatgggcatcgaggtgcccgagaccctggtgcacaagctgaccggcaagaaggtgaccagcaagaacggccagcccatcatccccgtgctgctgcccaagtacatcggcctggaccccgtggcccccggcgacctgaccatggtgatcacccaggactgcgacacctgccacagccccgccagcctgcccgccgtggtggagaagtga

SEQ ID NO: 112 - RNA, 912 nt, synthetic construct
  misc_feature 1..912: note = MARV VP40, Angola 2005,optimized mRNA Sequence
  source 1..912: mol_type = other RNA
atggccagcagcagcaactacaacacctacatgcagtacctgaacccccccccctacgccgaccacggcgccaaccagctgatccccgccgaccagctgagcaaccagcagggcatcacccccaactacgtgggcgacctgaacctggacgaccagttcaagggcaacgtgtgccacgccttcaccctggaggccatcatcgacatcagcgcctacaacgagagaaccgtgaagggcgtgcccgcctggctgcccctgggcatcatgagcaacttcgagtaccccctggcccacaccgtggccgccctgctgaccggcagctacaccatcacccagttcacccacaacggccagaagttcgtgagagtgaacagactgggcaccggcatccccgcccaccccctgagaatgctgagagagggcaaccaggccttcatccagaacatggtgatccccagaaacttcagcaccaaccagttcacctacaacctgaccaacctggtgctgagcgtgcagaagctgcccgacgacgcctggagacccagcaaggacaagctgatcggcaacaccatgcaccccgccgtgagcgtgcaccccaacctgccccccatcgtgctgcccaccgtgaagaagcaggcctacagacagcacaagaaccccaacaacggccccctgctggccatcagcggcatcctgcaccagctgagagtggagaaggtgcccgagaagaccagcctgttcagaatcagcctgcccgccgacatgttcagcgtgaaggagggcatgatgaagaagagaggcgagaacagccccgtggtgtacttccaggcccccgagaacttccccctgaacggcttcaacaacagacaggtggtgctggcctacgccaaccccaccctgagcgccgtgtga

SEQ ID NO: 113 - RNA, 2220 nt, synthetic construct
  misc_feature 1..2220: note = EBOV NP, Zaire 1976, optimized mRNA Sequence
  source 1..2220: mol_type = other RNA
atggacagcagaccccagaagatctggatggcccccagcctgaccgagagcgacatggactaccacaagatcctgaccgccggcctgagcgtgcagcagggcatcgtgagacagagagtgatccccgtgtaccaggtgaacaacctggaggagatctgccagctgatcatccaggccttcgaggccggcgtggacttccaggagagcgccgacagcttcctgctgatgctgtgcctgcaccacgcctaccagggcgactacaagctgttcctggagagcggcgccgtgaagtacctggagggccacggcttcagattcgaggtgaagaagagagacggcgtgaagagactggaggagctgctgcccgccgtgagcagcggcaagaacatcaagagaaccctggccgccatgcccgaggaggagaccaccgaggccaacgccggccagttcctgagcttcgccagcctgttcctgcccaagctggtggtgggcgagaaggcctgcctggagaaggtgcagagacagatccaggtgcacgccgagcagggcctgatccagtaccccaccgcctggcagagcgtgggccacatgatggtgatcttcagactgatgagaaccaacttcctgatcaagttcctgctgatccaccagggcatgcacatggtggccggccacgacgccaacgacgccgtgatcagcaacagcgtggcccaggccagattcagcggcctgctgatcgtgaagaccgtgctggaccacatcctgcagaagaccgagagaggcgtgagactgcaccccctggccagaaccgccaaggtgaagaacgaggtgaacagcttcaaggccgccctgagcagcctggccaagcacggcgagtacgcccccttcgccagactgctgaacctgagcggcgtgaacaacctggagcacggcctgttcccccagctgagcgccatcgccctgggcgtggccaccgcccacggcagcaccctggccggcgtgaacgtgggcgagcagtaccagcagctgagagaggccgccaccgaggccgagaagcagctgcagcagtacgccgagagcagagagctggaccacctgggcctggacgaccaggagaagaagatcctgatgaacttccaccagaagaagaacgagatcagcttccagcagaccaacgccatggtgaccctgagaaaggagagactggccaagctgaccgaggccatcaccgccgccagcctgcccaagaccagcggccactacgacgacgacgacgacatccccttccccggccccatcaacgacgacgacaaccccggccaccaggacgacgaccccaccgacagccaggacaccaccatccccgacgtggtggtggaccccgacgacggcagctacggcgagtaccagagctacagcgagaacggcatgaacgcccccgacgacctggtgctgttcgacctggacgaggacgacgaggacaccaagcccgtgcccaacagaagcaccaagggcggccagcagaagaacagccagaagggccagcacatcgagggcagacagacccagagcagacccatccagaacgtgcccggcccccacagaaccatccaccacgccagcgcccccctgaccgacaacgacagaagaaacgagcccagcggcagcaccagccccagaatgctgacccccatcaacgaggaggccgaccccctggacgacgccgacgacgagaccagcagcctgccccccctggagagcgacgacgaggagcaggacagagacggcaccagcaacagaacccccaccgtggccccccccgcccccgtgtacagagaccacagcgagaagaaggagctgccccaggacgagcagcaggaccaggaccacacccaggaggccagaaaccaggacagcgacaacacccagagcgagcacagcttcgaggagatgtacagacacatcctgagaagccagggccccttcgacgccgtgctgtactaccacatgatgaaggacgagcccgtggtgttcagcaccagcgacggcaaggagtacacctaccccgacagcctggaggaggagtaccccccctggctgaccgagaaggaggccatgaacgaggagaacagattcgtgaccctggacggccagcagttctactggcccgtgatgaaccacaagaacaagttcatggccatcctgcagcaccaccagtga

SEQ ID NO: 114 - RNA, 2220 nt, synthetic construct
  misc_feature 1..2220: note = EBOV NP, Sierra Leone 2014, optimized mRNA Sequence
  source 1..2220: mol_type = other RNA
atggacagcagaccccagaaggtgtggatgacccccagcctgaccgagagcgacatggactaccacaagatcctgaccgccggcctgagcgtgcagcagggcatcgtgagacagagagtgatccccgtgtaccaggtgaacaacctggaggagatctgccagctgatcatccaggccttcgaggccggcgtggacttccaggagagcgccgacagcttcctgctgatgctgtgcctgcaccacgcctaccagggcgactacaagctgttcctggagagcggcgccgtgaagtacctggagggccacggcttcagattcgaggtgaagaagtgcgacggcgtgaagagactggaggagctgctgcccgccgtgagcagcggcagaaacatcaagagaaccctggccgccatgcccgaggaggagaccaccgaggccaacgccggccagttcctgagcttcgccagcctgttcctgcccaagctggtggtgggcgagaaggcctgcctggagaaggtgcagagacagatccaggtgcacgccgagcagggcctgatccagtaccccaccgcctggcagagcgtgggccacatgatggtgatcttcagactgatgagaaccaacttcctgatcaagttcctgctgatccaccagggcatgcacatggtggccggccacgacgccaacgacgccgtgatcagcaacagcgtggcccaggccagattcagcggcctgctgatcgtgaagaccgtgctggaccacatcctgcagaagaccgagagaggcgtgagactgcaccccctggccagaaccgccaaggtgaagaacgaggtgaacagcttcaaggccgccctgagcagcctggccaagcacggcgagtacgcccccttcgccagactgctgaacctgagcggcgtgaacaacctggagcacggcctgttcccccagctgagcgccatcgccctgggcgtggccaccgcccacggcagcaccctggccggcgtgaacgtgggcgagcagtaccagcagctgagagaggccgccaccgaggccgagaagcagctgcagcagtacgccgagagcagagagctggaccacctgggcctggacgaccaggagaagaagatcctgatgaacttccaccagaagaagaacgagatcagcttccagcagaccaacgccatggtgaccctgagaaaggagagactggccaagctgaccgaggccatcaccgccgccagcctgcccaagaccagcggccactacgacgacgacgacgacatccccttccccggccccatcaacgacgacgacaaccccggccaccaggacgacgaccccaccgacagccaggacaccaccatccccgacgtggtggtggaccccgacgacggcggctacggcgagtaccagagctacagcgagaacggcatgagcgcccccgacgacctggtgctgttcgacctggacgaggacgacgaggacaccaagcccgtgcccaacagaagcaccaagggcggccagcagaagaacagccagaagggccagcacaccgagggcagacagacccagagcacccccacccagaacgtgaccggccccagaagaaccatccaccacgccagcgcccccctgaccgacaacgacagaagaaacgagcccagcggcagcaccagccccagaatgctgacccccatcaacgaggaggccgaccccctggacgacgccgacgacgagaccagcagcctgccccccctggagagcgacgacgaggagcaggacagagacggcaccagcaacagaacccccaccgtggccccccccgcccccgtgtacagagaccacagcgagaagaaggagctgccccaggacgagcagcaggaccaggaccacatccaggaggccagaaaccaggacagcgacaacacccagcccgagcacagcttcgaggagatgtacagacacatcctgagaagccagggccccttcgacgccgtgctgtactaccacatgatgaaggacgagcccgtggtgttcagcaccagcgacggcaaggagtacacctaccccgacagcctggaggaggagtaccccccctggctgaccgagaaggaggccatgaacgacgagaacagattcgtgaccctggacggccagcagttctactggcccgtgatgaaccacagaaacaagttcatggccatcctgcagcaccaccagtga

SEQ ID NO: 115 - RNA, 2031 nt, synthetic construct
  misc_feature 1..2031: note = BDBV GP, Uganda 2007,optimized mRNA Sequence
  source 1..2031: mol_type = other RNA
atggtgaccagcggcatcctgcagctgcccagagagagattcagaaagaccagcttcttcgtgtgggtgatcatcctgttccacaaggtgttccccatccccctgggcgtggtgcacaacaacaccctgcaggtgagcgacatcgacaagctggtgtgcagagacaagctgagcagcaccagccagctgaagagcgtgggcctgaacctggagggcaacggcgtggccaccgacgtgcccaccgccaccaagagatggggcttcagagccggcgtgccccccaaggtggtgaactacgaggccggcgagtgggccgagaactgctacaacctggacatcaagaaggccgacggcagcgagtgcctgcccgaggcccccgagggcgtgagaggcttccccagatgcagatacgtgcacaaggtgagcggcaccggcccctgccccgagggctacgccttccacaaggagggcgccttcttcctgtacgacagactggccagcaccatcatctacagaagcaccaccttcagcgagggcgtggtggccttcctgatcctgcccgagaccaagaaggacttcttccagagcccccccctgcacgagcccgccaacatgaccaccgaccccagcagctactaccacaccgtgaccctgaactacgtggccgacaacttcggcaccaacatgaccaacttcctgttccaggtggaccacctgacctacgtgcagctggagcccagattcaccccccagttcctggtgcagctgaacgagaccatctacaccaacggcagaagaagcaacaccaccggcaccctgatctggaaggtgaaccccaccgtggacaccggcgtgggcgagtgggccttctgggagaacaagaagaacttcaccaagaccctgagcagcgaggagctgagcgtgatcttcgtgcccagagcccaggaccccggcagcaaccagaagaccaaggtgacccccaccagcttcgccaacaaccagaccagcaagaaccacgaggacctggtgcccgaggaccccgccagcgtggtgcaggtgagagacctgcagagagagaacaccgtgcccacccccccccccgacaccgtgcccaccaccctgatccccgacaccatggaggagcagaccaccagccactacgagccccccaacatcagcagaaaccaccaggagagaaacaacaccgcccaccccgagaccctggccaacaacccccccgacaacaccacccccagcacccccccccaggacggcgagagaaccagcagccacaccacccccagccccagacccgtgcccaccagcaccatccaccccaccaccagagagacccacatccccaccaccatgaccaccagccacgacaccgacagcaacagacccaaccccatcgacatcagcgagagcaccgagcccggccccctgaccaacaccaccagaggcgccgccaacctgctgaccggcagcagaagaaccagaagagagatcaccctgagaacccaggccaagtgcaaccccaacctgcactactggaccacccaggacgagggcgccgccatcggcctggcctggatcccctacttcggccccgccgccgagggcatctacaccgagggcatcatgcacaaccagaacggcctgatctgcggcctgagacagctggccaacgagaccacccaggccctgcagctgttcctgagagccaccaccgagctgagaaccttcagcatcctgaacagaaaggccatcgacttcctgctgcagagatggggcggcacctgccacatcctgggccccgactgctgcatcgagccccacgactggaccaagaacatcaccgacaagatcgaccagatcatccacgacttcatcgacaagcccctgcccgaccagaccgacaacgacaactggtggaccggctggagacagtgggtgcccgccggcatcggcatcaccggcgtgatcatcgccgtgatcgccctgctgtgcatctgcaagttcctgctgtga

SEQ ID NO: 116 - RNA, 2031 nt, synthetic construct
  misc_feature 1..2031: note = SUDV GP, Gulu, Uganda 2007, optimized mRNA Sequence
  source 1..2031: mol_type = other RNA
atgggcggcctgagcctgctgcagctgcccagagacaagttcagaaagagcagcttcttcgtgtgggtgatcatcctgttccagaaggccttcagcatgcccctgggcgtggtgaccaacagcaccctggaggtgaccgagatcgaccagctggtgtgcaaggaccacctggccagcaccgaccagctgaagagcgtgggcctgaacctggagggcagcggcgtgagcaccgacatccccagcgccaccaagagatggggcttcagaagcggcgtgccccccaaggtggtgagctacgaggccggcgagtgggccgagaactgctacaacctggagatcaagaagcccgacggcagcgagtgcctgccccccccccccgacggcgtgagaggcttccccagatgcagatacgtgcacaaggcccagggcaccggcccctgccccggcgactacgccttccacaaggacggcgccttcttcctgtacgacagactggccagcaccgtgatctacagaggcgtgaacttcgccgagggcgtgatcgccttcctgatcctggccaagcccaaggagaccttcctgcagagcccccccatcagagaggccgtgaactacaccgagaacaccagcagctactacgccaccagctacctggagtacgagatcgagaacttcggcgcccagcacagcaccaccctgttcaagatcgacaacaacaccttcgtgagactggacagaccccacaccccccagttcctgttccagctgaacgacaccatccacctgcaccagcagctgagcaacaccaccggcagactgatctggaccctggacgccaacatcaacgccgacatcggcgagtgggccttctgggagaacaagaagaacctgagcgagcagctgagaggcgaggagctgagcttcgaggccctgagcctgaacgagaccgaggacgacgacgccgccagcagcagaatcaccaagggcagaatcagcgacagagccaccagaaagtacagcgacctggtgcccaagaacagccccggcatggtgcccctgcacatccccgagggcgagaccaccctgcccagccagaacagcaccgagggcagaagagtgggcgtgaacacccaggagaccatcaccgagaccgccgccaccatcatcggcaccaacggcaaccacatgcagatcagcaccatcggcatcagacccagcagcagccagatccccagcagcagccccaccaccgcccccagccccgaggcccagacccccaccacccacaccagcggccccagcgtgatggccaccgaggagcccaccaccccccccggcagcagccccggccccaccaccgaggcccccaccctgaccacccccgagaacatcaccaccgccgtgaagaccgtgctgccccaggagagcaccagcaacggcctgatcaccagcaccgtgaccggcatcctgggcagcctgggcctgagaaagagaagcagaagacagaccaacaccaaggccaccggcaagtgcaaccccaacctgcactactggaccgcccaggagcagcacaacgccgccggcatcgcctggatcccctacttcggccccggcgccgagggcatctacaccgagggcctgatgcacaaccagaacgccctggtgtgcggcctgagacagctggccaacgagaccacccaggccctgcagctgttcctgagagccaccaccgagctgagaacctacaccatcctgaacagaaaggccatcgacttcctgctgagaagatggggcggcacctgcagaatcctgggccccgactgctgcatcgagccccacgactggaccaagaacatcaccgacaagatcaaccagatcatccacgacttcatcgacaaccccctgcccaaccaggacaacgacgacaactggtggaccggctggagacagtggatccccgccggcatcggcatcaccggcatcatcatcgccatcatcgccctgctgtgcgtgtgcaagctgctgtgctga

SEQ ID NO: 117 - RNA, 2031 nt, synthetic construct
  misc_feature 1..2031: note = TAFV GP, Cote dIvoire 1994, optimized mRNA Sequence
  source 1..2031: mol_type = other RNA
atgggcgccagcggcatcctgcagctgcccagagagagattcagaaagaccagcttcttcgtgtgggtgatcatcctgttccacaaggtgttcagcatccccctgggcgtggtgcacaacaacaccctgcaggtgagcgacatcgacaagttcgtgtgcagagacaagctgagcagcaccagccagctgaagagcgtgggcctgaacctggagggcaacggcgtggccaccgacgtgcccaccgccaccaagagatggggcttcagagccggcgtgccccccaaggtggtgaactgcgaggccggcgagtgggccgagaactgctacaacctggccatcaagaaggtggacggcagcgagtgcctgcccgaggcccccgagggcgtgagagacttccccagatgcagatacgtgcacaaggtgagcggcaccggcccctgccccggcggcctggccttccacaaggagggcgccttcttcctgtacgacagactggccagcaccatcatctacagaggcaccaccttcgccgagggcgtgatcgccttcctgatcctgcccaaggccagaaaggacttcttccagagcccccccctgcacgagcccgccaacatgaccaccgaccccagcagctactaccacaccaccaccatcaactacgtggtggacaacttcggcaccaacaccaccgagttcctgttccaggtggaccacctgacctacgtgcagctggaggccagattcaccccccagttcctggtgctgctgaacgagaccatctacagcgacaacagaagaagcaacaccaccggcaagctgatctggaagatcaaccccaccgtggacaccagcatgggcgagtgggccttctgggagaacaagaagaacttcaccaagaccctgagcagcgaggagctgagcttcgtgcccgtgcccgagacccagaaccaggtgctggacaccaccgccaccgtgagcccccccatcagcgcccacaaccacgccgccgaggaccacaaggagctggtgagcgaggacagcacccccgtggtgcagatgcagaacatcaagggcaaggacaccatgcccaccaccgtgaccggcgtgcccaccaccacccccagccccttccccatcaacgccagaaacaccgaccacaccaagagcttcatcggcctggagggcccccaggaggaccacagcaccacccagcccgccaagaccaccagccagcccaccaacagcaccgagagcaccaccctgaaccccaccagcgagcccagcagcagaggcaccggccccagcagccccaccgtgcccaacaccaccgagagccacgccgagctgggcaagaccacccccaccaccctgcccgagcagcacaccgccgccagcgccatccccagagccgtgcaccccgacgagctgagcggccccggcttcctgaccaacaccatcagaggcgtgaccaacctgctgaccggcagcagaagaaagagaagagacgtgacccccaacacccagcccaagtgcaaccccaacctgcactactggaccgccctggacgagggcgccgccatcggcctggcctggatcccctacttcggccccgccgccgagggcatctacaccgagggcatcatggagaaccagaacggcctgatctgcggcctgagacagctggccaacgagaccacccaggccctgcagctgttcctgagagccaccaccgagctgagaaccttcagcatcctgaacagaaaggccatcgacttcctgctgcagagatggggcggcacctgccacatcctgggccccgactgctgcatcgagccccaggactggaccaagaacatcaccgacaagatcgaccagatcatccacgacttcgtggacaacaacctgcccaaccagaacgacggcagcaactggtggaccggctggaagcagtgggtgcccgccggcatcggcatcaccggcgtgatcatcgccatcatcgccctgctgtgcatctgcaagttcatgctgtga

SEQ ID NO: 118 - RNA, 981 nt, synthetic construct
  misc_feature 1..981: note = BDBV VP40, Uganda 2007, optimized mRNA Sequence
  source 1..981: mol_type = other RNA
atgagaagagccatcctgcccaccgccccccccgagtacatcgaggccgtgtaccccatgagaaccgtgagcaccagcatcaacagcaccgccagcggccccaacttccccgcccccgacgtgatgatgagcgacacccccagcaacagcctgagacccatcgccgacgacaacatcgaccaccccagccacacccccaccagcgtgagcagcgccttcatcctggaggccatggtgaacgtgatcagcggccccaaggtgctgatgaagcagatccccatctggctgcccctgggcgtggccgaccagaagacctacagcttcgacagcaccaccgccgccatcatgctggccagctacaccatcacccacttcggcaagaccagcaaccccctggtgagaatcaacagactgggccccggcatccccgaccaccccctgagactgctgagaatcggcaaccaggccttcctgcaggagttcgtgctgccccccgtgcagctgccccagtacttcaccttcgacctgaccgccctgaagctgatcacccagcccctgcccgccgccacctggaccgacgacacccccaccggccccaccggcatcctgagacccggcatcagcttccaccccaagctgagacccatcctgctgcccggcaagaccggcaagagaggcagcagcagcgacctgaccagccccgacaagatccaggccatcatgaacttcctgcaggacctgaagctggtgcccatcgaccccgccaagaacatcatgggcatcgaggtgcccgagctgctggtgcacagactgaccggcaagaagatcaccaccaagaacggccagcccatcatccccatcctgctgcccaagtacatcggcatggaccccatcagccagggcgacctgaccatggtgatcacccaggactgcgacacctgccacagccccgccagcctgccccccgtgagcgagaagtga

SEQ ID NO: 119 - RNA, 981 nt, synthetic construct
  misc_feature 1..981: note = SUDV VP40, Gulu, Uganda 2000, optimized mRNA Sequence
  source 1..981: mol_type = other RNA
atgagaagagtgaccgtgcccaccgccccccccgcctacgccgacatcggctaccccatgagcatgctgcccatcaagagcagcagagccgtgagcggcatccagcagaagcaggaggtgctgcccggcatggacacccccagcaacagcatgagacccgtggccgacgacaacatcgaccacaccagccacacccccaacggcgtggccagcgccttcatcctggaggccaccgtgaacgtgatcagcggccccaaggtgctgatgaagcagatccccatctggctgcccctgggcatcgccgaccagaagacctacagcttcgacagcaccaccgccgccatcatgctggccagctacaccatcacccacttcggcaaggccaacaaccccctggtgagagtgaacagactgggccagggcatccccgaccaccccctgagactgctgagaatgggcaaccaggccttcctgcaggagttcgtgctgccccccgtgcagctgccccagtacttcaccttcgacctgaccgccctgaagctggtgacccagcccctgcccgccgccacctggaccgacgagacccccagcaacctgagcggcgccctgagacccggcctgagcttccaccccaagctgagacccgtgctgctgcccggcaagaccggcaagaagggccacgtgagcgacctgaccgcccccgacaagatccagaccatcgtgaacctgatgcaggacttcaagatcgtgcccatcgaccccgccaagagcatcatcggcatcgaggtgcccgagctgctggtgcacaagctgaccggcaagaagatgagccagaagaacggccagcccatcatccccgtgctgctgcccaagtacatcggcctggaccccatcagccccggcgacctgaccatggtgatcacccccgactacgacgactgccacagccccgccagctgcagctacctgagcgagaagtga

SEQ ID NO: 120 - RNA, 981 nt, synthetic construct
  misc_feature 1..981: note = TAFV VP40, Cote dIvoire 1994, optimized mRNA Sequence
  source 1..981: mol_type = other RNA
atgagaagaatcatcctgcccaccgccccccccgagtacatggaggccgtgtaccccatgagaaccatgaacagcggcgccgacaacaccgccagcggccccaactacaccaccaccggcgtgatgaccaacgacacccccagcaacagcctgagacccgtggccgacgacaacatcgaccaccccagccacacccccaacagcgtggccagcgccttcatcctggaggccatggtgaacgtgatcagcggccccaaggtgctgatgaagcagatccccatctggctgcccctgggcgtgagcgaccagaagacctacagcttcgacagcaccaccgccgccatcatgctggccagctacaccatcacccacttcggcaagaccagcaaccccctggtgagaatcaacagactgggccccggcatccccgaccaccccctgagactgctgagaatcggcaaccaggccttcctgcaggagttcgtgctgccccccgtgcagctgccccagtacttcaccttcgacctgaccgccctgaagctgatcacccagcccctgcccgccgccacctggaccgacgagacccccgccgtgagcaccggcaccctgagacccggcatcagcttccaccccaagctgagacccatcctgctgcccggcagagccggcaagaagggcagcaacagcgacctgaccagccccgacaagatccaggccatcatgaacttcctgcaggacctgaagatcgtgcccatcgaccccaccaagaacatcatgggcatcgaggtgcccgagctgctggtgcacagactgaccggcaagaagaccaccaccaagaacggccagcccatcatccccatcctgctgcccaagtacatcggcctggaccccctgagccagggcgacctgaccatggtgatcacccaggactgcgacagctgccacagccccgccagcctgccccccgtgaacgagaagtga

SEQ ID NO: 121 - RNA, 2088 nt, synthetic construct
  misc_feature 1..2088: note = MARV NP, Angola 2005, optimized mRNA Sequence
  source 1..2088: mol_type = other RNA
atggacctgcacagcctgctggagctgggcaccaagcccaccgccccccacgtgagaaacaagaaggtgatcctgttcgacaccaaccaccaggtgagcatctgcaaccagatcatcgacgccatcaacagcggcatcgacctgggcgacctgctggagggcggcctgctgaccctgtgcgtggagcactactacaacagcgacaaggacaagttcaacaccagccccatcgccaagtacctgagagacgccggctacgagttcgacgtgatcaagaacgccgacgccaccagattcctggacgtgatccccaacgagccccactacagccccctgatcctggccctgaagaccctggagagcaccgagagccagagaggcagaatcggcctgttcctgagcttctgcagcctgttcctgcccaagctggtggtgggcgacagagccagcatcgagaaggccctgagacaggtgaccgtgcaccaggagcagggcatcgtgacctaccccaaccactggctgaccaccggccacatgaaggtgatcttcggcatcctgagaagcagcttcatcctgaagttcgtgctgatccaccagggcgtgaacctggtgaccggccacgacgcctacgacagcatcatcagcaacagcgtgggccagaccagattcagcggcctgctgatcgtgaagaccgtgctggagttcatcctgcagaagaccgacagcggcgtgaccctgcaccccctggtgagaaccagcaaggtgaagaacgaggtggccagcttcaagcaggccctgagcaacctggccagacacggcgagtacgcccccttcgccagagtgctgaacctgagcggcatcaacaacctggagcacggcctgtacccccagctgagcgccatcgccctgggcgtggccaccgcccacggcagcaccctggccggcgtgaacgtgggcgagcagtaccagcagctgagagaggccgcccacgacgccgaggtgaagctgcagagaagacacgagcaccaggagatccaggccatcgccgaggacgacgaggagagaaagatcctggagcagttccacctgcagaagaccgagatcacccacagccagaccctggccgtgctgagccagaagagagagaagctggccagactggccgccgagatcgagaacaacatcgtggaggaccagggcttcaagcagagccagaacagagtgagccagagcttcctgaacgaccccacccccgtggaggtgaccgtgcaggccagacccatcaacagacccaccgccctgcccccccccgtggacagcaagatcgagcacgagagcaccgaggacagcagcagcagcagcagcttcgtggacctgaacgaccccttcgccctgctgaacgaggacgaggacaccctggacgacagcgtgatgatccccagcaccaccagcagagagttccagggcatccccgagccccccagacagagccaggacatcgacaacagccagggcaagcaggaggacgagagcaccaacctgatcaagaagcccttcctgagataccaggagctgccccccgtgcaggaggacgacgagagcgagtacaccaccgacagccaggagagcatcgaccagcccggcagcgacaacgagcagggcgtggacctgccccccccccccctgtacgcccaggagaagagacaggaccccatccagcaccccgccgtgagcagccaggaccccttcggcagcatcggcgacgtgaacggcgacatcctggagcccatcagaagccccagcagccccagcgccccccaggaggacaccagagccagagaggcctacgagctgagccccgacttcaccaactacgaggacaaccagcagaactggccccagagagtggtgaccaagaagggcagaaccttcctgtaccccaacgacctgctgcagaccaacccccccgagagcctgatcaccgccctggtggaggagtaccagaaccccgtgagcgccaaggagctgcaggccgactggcccgacatgagcttcgacgagagaagacacgtggccatgaacctgtga

SEQ ID NO: 122 - RNA, 2220 nt, synthetic construct
  misc_feature 1..2220: note = BDBV NP, Uganda 2007, optimized mRNA Sequence
  source 1..2220: mol_type = other RNA
atggaccccagacccatcagaacctggatgatgcacaacaccagcgaggtggaggccgactaccacaagatcctgaccgccggcctgagcgtgcagcagggcatcgtgagacagagaatcatccccgtgtaccagatcagcaacctggaggaggtgtgccagctgatcatccaggccttcgaggccggcgtggacttccaggacagcgccgacagcttcctgctgatgctgtgcctgcaccacgcctaccagggcgactacaagcagttcctggagagcaacgccgtgaagtacctggagggccacggcttcagattcgagatgaagaagaaggagggcgtgaagagactggaggagctgctgcccgccgccagcagcggcaagaacatcaagagaaccctggccgccatgcccgaggaggagaccaccgaggccaacgccggccagttcctgagcttcgccagcctgttcctgcccaagctggtggtgggcgagaaggcctgcctggagaaggtgcagagacagatccaggtgcacgccgagcagggcctgatccagtaccccaccagctggcagagcgtgggccacatgatggtgatcttcagactgatgagaaccaacttcctgatcaagttcctgctgatccaccagggcatgcacatggtggccggccacgacgccaacgacgccgtgatcgccaacagcgtggcccaggccagattcagcggcctgctgatcgtgaagaccgtgctggaccacatcctgcagaagaccgagcacggcgtgagactgcaccccctggccagaaccgccaaggtgaagaacgaggtgagcagcttcaaggccgccctggccagcctggcccagcacggcgagtacgcccccttcgccagactgctgaacctgagcggcgtgaacaacctggagcacggcctgttcccccagctgagcgccatcgccctgggcgtggccaccgcccacggcagcaccctggccggcgtgaacgtgggcgagcagtaccagcagctgagagaggccgccaccgaggccgagaagcagctgcagaagtacgccgagagcagagagctggaccacctgggcctggacgaccaggagaagaagatcctgaaggacttccaccagaagaagaacgagatcagcttccagcagaccaccgccatggtgaccctgagaaaggagagactggccaagctgaccgaggccatcaccagcaccagcatcctgaagaccggcagaagatacgacgacgacaacgacatccccttccccggccccatcaacgacaacgagaacagcggccagaacgacgacgaccccaccgacagccaggacaccaccatccccgacgtgatcatcgaccccaacgacggcggctacaacaactacagcgactacgccaacgacgccgccagcgcccccgacgacctggtgctgttcgacctggaggacgaggacgacgccgacaaccccgcccagaacacccccgagaagaacgacagacccgccaccaccaagctgagaaacggccaggaccaggacggcaaccagggcgagaccgccagccccagagtggcccccaaccagtacagagacaagcccatgccccaggtgcaggacagaagcgagaaccacgaccagaccctgcagacccagagcagagtgctgacccccatcagcgaggaggccgaccccagcgaccacaacgacggcgacaacgagagcatcccccccctggagagcgacgacgagggcagcaccgacaccaccgccgccgagaccaagcccgccaccgccccccccgcccccgtgtacagaagcatcagcgtggacgacagcgtgcccagcgagaacatccccgcccagagcaaccagaccaacaacgaggacaacgtgagaaacaacgcccagagcgagcagagcatcgccgagatgtaccagcacatcctgaagacccagggccccttcgacgccatcctgtactaccacatgatgaaggaggagcccatcatcttcagcaccagcgacggcaaggagtacacctaccccgacagcctggaggacgagtaccccccctggctgagcgagaaggaggccatgaacgaggacaacagattcatcaccatggacggccagcagttctactggcccgtgatgaaccacagaaacaagttcatggccatcctgcagcaccacagatga

SEQ ID NO: 123 - RNA, 2217 nt, synthetic construct
  misc_feature 1..2217: note = SUDV NP, Gulu, Uganda 2000,optimized mRNA Sequence
  source 1..2217: mol_type = other RNA
atggacaagagagtgagaggcagctgggccctgggcggccagagcgaggtggacctggactaccacaagatcctgaccgccggcctgagcgtgcagcagggcatcgtgagacagagagtgatccccgtgtacgtggtgagcgacctggagggcatctgccagcacatcatccaggccttcgaggccggcgtggacttccaggacaacgccgacagcttcctgctgctgctgtgcctgcaccacgcctaccagggcgaccacagactgttcctgaagagcgacgccgtgcagtacctggagggccacggcttcagattcgaggtgagagagaaggagaacgtgcacagactggacgagctgctgcccaacgtgaccggcggcaagaacctgagaagaaccctggccgccatgcccgaggaggagaccaccgaggccaacgccggccagttcctgagcttcgccagcctgttcctgcccaagctggtggtgggcgagaaggcctgcctggagaaggtgcagagacagatccaggtgcacgccgagcagggcctgatccagtaccccaccagctggcagagcgtgggccacatgatggtgatcttcagactgatgagaaccaacttcctgatcaagttcctgctgatccaccagggcatgcacatggtggccggccacgacgccaacgacaccgtgatcagcaacagcgtggcccaggccagattcagcggcctgctgatcgtgaagaccgtgctggaccacatcctgcagaagaccgacctgggcgtgagactgcaccccctggccagaaccgccaaggtgaagaacgaggtgagcagcttcaaggccgccctgggcagcctggccaagcacggcgagtacgcccccttcgccagactgctgaacctgagcggcgtgaacaacctggagcacggcctgtacccccagctgagcgccatcgccctgggcgtggccaccgcccacggcagcaccctggccggcgtgaacgtgggcgagcagtaccagcagctgagagaggccgccaccgaggccgagaagcagctgcagcagtacgccgagaccagagagctggacaacctgggcctggacgagcaggagaagaagatcctgatgagcttccaccagaagaagaacgagatcagcttccagcagaccaacgccatggtgaccctgagaaaggagagactggccaagctgaccgaggccatcaccaccgccagcaagatcaaggtgggcgacagataccccgacgacaacgacatccccttccccggccccatctacgacgagacccaccccaaccccagcgacgacaaccccgacgacagcagagacaccaccatccccggcggcgtggtggacccctacgacgacgagagcaacaactaccccgactacgaggacagcgccgagggcaccaccggcgacctggacctgttcaacctggacgacgacgacgacgacagccagcccggcccccccgacagaggccagagcaaggagagagccgccagaacccacggcctgcaggaccccaccctggacggcgccaagaaggtgcccgagctgacccccggcagccaccagcccggcaacctgcacatcaccaagcccggcagcaacaccaaccagccccagggcaacatgagcagcaccctgcagagcatgacccccatccaggaggagagcgagcccgacgaccagaaggacgacgacgacgagagcctgaccagcctggacagcgagggcgacgaggacgtggagagcgtgagcggcgagaacaaccccaccgtggccccccccgcccccgtgtacaaggacaccggcgtggacaccaaccagcagaacggccccagcaacgccgtggacggccagggcagcgagagcgaggccctgcccatcaaccccgagaagggcagcgccctggaggagacctactaccacctgctgaagacccagggccccttcgaggccatcaactactaccacctgatgagcgacgagcccatcgccttcagcaccgagagcggcaaggagtacatcttccccgacagcctggaggaggcctaccccccctggctgagcgagaaggaggccctggagaaggagaacagatacctggtgatcgacggccagcagttcctgtggcccgtgatgagcctgcaggacaagttcctggccgtgctgcagcacgactga

SEQ ID NO: 124 - RNA, 2220 nt, synthetic construct
  misc_feature 1..2220: note = TAFV NP, Cote dIvoire 1994,optimized mRNA Sequence
  source 1..2220: mol_type = other RNA
atggagagcagagcccacaaggcctggatgacccacaccgccagcggcttcgagaccgactaccacaagatcctgaccgccggcctgagcgtgcagcagggcatcgtgagacagagagtgatccaggtgcaccaggtgaccaacctggaggagatctgccagctgatcatccaggccttcgaggccggcgtggacttccaggagagcgccgacagcttcctgctgatgctgtgcctgcaccacgcctaccagggcgactacaagcagttcctggagagcaacgccgtgaagtacctggagggccacggcttcagattcgaggtgagaaagaaggagggcgtgaagagactggaggagctgctgcccgccgccagcagcggcaagagcatcagaagaaccctggccgccatgcccgaggaggagaccaccgaggccaacgccggccagttcctgagcttcgccagcctgttcctgcccaagctggtggtgggcgagaaggcctgcctggagaaggtgcagagacagatccaggtgcacagcgagcagggcctgatccagtaccccaccgcctggcagagcgtgggccacatgatggtgatcttcagactgatgagaaccaacttcctgatcaagttcctgctgatccaccagggcatgcacatggtggccggccacgacgccaacgacgccgtgatcgccaacagcgtggcccaggccagattcagcggcctgctgatcgtgaagaccgtgctggaccacatcctgcagaagaccgagcacggcgtgagactgcaccccctggccagaaccgccaaggtgaagaacgaggtgaacagcttcaaggccgccctgagcagcctggcccagcacggcgagtacgcccccttcgccagactgctgaacctgagcggcgtgaacaacctggagcacggcctgttcccccagctgagcgccatcgccctgggcgtggccaccgcccacggcagcaccctggccggcgtgaacgtgggcgagcagtaccagcagctgagagaggccgccaccgaggccgagaagcagctgcagaagtacgccgagagcagagagctggaccacctgggcctggacgaccaggagaagaagatcctgaaggacttccaccagaagaagaacgagatcagcttccagcagaccaccgccatggtgaccctgagaaaggagagactggccaagctgaccgaggccatcaccagcaccagcctgctgaagaccggcaagcagtacgacgacgacaacgacatccccttccccggccccatcaacgacaacgagaacagcgagcagcaggacgacgaccccaccgacagccaggacaccaccatccccgacatcatcgtggaccccgacgacggcagatacaacaactacggcgactaccccagcgagaccgccaacgcccccgaggacctggtgctgttcgacctggaggacggcgacgaggacgaccacagacccagcagcagcagcgagaacaacaacaagcacagcctgaccggcaccgacagcaacaagaccagcaactggaacagaaaccccaccaacatgcccaagaaggacagcacccagaacaacgacaaccccgcccagagagcccaggagtacgccagagacaacatccaggacacccccaccccccacagagccctgacccccatcagcgaggagaccggcagcaacggccacaacgaggacgacatcgacagcatcccccccctggagagcgacgaggagaacaacaccgagaccaccatcaccaccaccaagaacaccaccgccccccccgcccccgtgtacagaagcaacagcgagaaggagcccctgccccaggagaagagccagaagcagcccaaccaggtgagcggcagcgagaacaccgacaacaagccccacagcgagcagagcgtggaggagatgtacagacacatcctgcagacccagggccccttcgacgccatcctgtactactacatgatgaccgaggagcccatcgtgttcagcaccagcgacggcaaggagtacgtgtaccccgacagcctggagggcgagcaccccccctggctgagcgagaaggaggccctgaacgaggacaacagattcatcaccatggacgaccagcagttctactggcccgtgatgaaccacagaaacaagttcatggccatcctgcagcaccacaagtga

SEQ ID NO: 125 - RNA, 2031 nt, synthetic construct
  misc_feature 1..2031: note = EBOV GP, Mayinga, Zaire 1976, optimized mRNA Sequence
  source 1..2031: mol_type = other RNA
atgggcgtgaccgggatcctgcagctccccagagacaggttcaagcggacaagctttttcctttgggtcattatactgtttcaacgcactttctccatccctttgggagttattcacaactctacgttacaggtgtcagatgtagacaaactggtgtgccgagataagctaagtagcaccaatcagctccgttccgtcggtctgaaccttgagggcaatggggtggccacagacgttccatctgctactaaaagatggggatttaggtcgggcgtgccgcccaaggtcgtgaactacgaagcaggtgagtgggccgaaaattgttataacctggagatcaaaaagcctgatgggtcagaatgcttgccagcggctcccgacggaattcggggcttccctcgctgtagatacgtacataaggttagcggcaccgggccatgcgccggagattttgcattccacaaagagggtgcctttttcctctatgacaggctggctagtacagtgatctaccggggcaccacttttgcagagggggtcgtggccttcctgatactcccccaggctaagaaagattttttctcctctcatcctttacgcgaaccagtgaatgccacagaggacccgagctccggatattactcaaccacgatccgatatcaagcgactggcttcggtaccaacgaaacagagtacctgtttgaggtcgataatcttacttacgttcagttggaaagtagattcaccccccagtttctgctacagctcaacgagacaatttataccagcgggaagaggtctaatacgactggaaaactgatctggaaggtgaaccctgaaattgacacaaccatcggcgagtgggcattctgggaaactaagaaaaatcttacacggaagatacgttccgaggagctgtcatttaccgtagtgagcaacggcgccaaaaatattagtgggcaatctccagctcgcacgtccagcgatcccggaaccaacacaactaccgaagaccacaagatcatggcctcggagaactcttccgcaatggtccaggtgcattcacagggtagagaagctgccgttagtcacttgacaactctcgcgaccattagcacatcccctcagtctctgaccactaaaccaggccccgacaatagcacgcacaacacaccggtgtacaagttagatatctcagaggccacccaagtcgaacagcatcacaggcggactgacaatgatagtaccgcatccgacacacctagcgctaccactgccgctgggcccccaaaagccgagaacacaaatacgtctaagtcaaccgatttcctggaccctgcaactacaaccagtccccagaaccatagcgagacagccggaaataacaatactcaccaccaagataccggcgaagagtccgcttcttcgggtaagctcgggcttatcacgaacaccattgcgggcgtggcaggactgataacaggcgggcgacgcactagaagggaagccatcgtaaatgctcagccaaaatgtaaccccaatttgcattattggaccacacaggacgagggagccgcaattggtctagcctggatcccttactttggcccagctgccgaagggatatatattgagggactgatgcacaaccaggatggcctcatctgcggtctgcggcaacttgcaaatgagactacccaggcgctgcagttgttcctccgcgctacgacagaactgagaacctttagcatcttaaacaggaaggccattgacttcctgctacagcgttgggggggcacttgtcatatcctcggaccggattgctgtattgagccccacgactggacaaaaaacataaccgacaagatcgatcaaattatccatgactttgttgataaaactctgcctgaccagggcgataatgacaactggtggaccgggtggcggcagtggatcccagcaggaattggtgtgacaggcgtcataatcgccgtgattgctcttttctgcatctgtaagtttgtcttctga

SEQ ID NO: 126 - RNA, 2031 nt, synthetic construct
  misc_feature 1..2031: note = EBOV GP, Sierra Leone 2014, optimized mRNA Sequence
  source 1..2031: mol_type = other RNA
atgggcgtgaccgggatcctgcagctccccagagacaggttcaagcggacatcctttttcctttgggtcattatactgtttcaacgcactttctccatccctttgggagttattcacaactctacgttacaggtgtcagatgtagacaaactggtgtgccgagataagctaagtagcaccaatcagctccgttccgtcggtctgaaccttgagggcaatggggtggccacagacgttccatctgtgactaaaagatggggatttaggtcgggcgtcccgcccaaggtggtaaactacgaagctggtgagtgggcagaaaattgttataacctggagatcaaaaagcctgatgggtcagaatgcttgccagccgcgcccgacggaatccggggcttccctcgctgtagatacgttcataaggtgagcggcaccgggccatgcgctggagattttgccttccacaaagagggtgcatttttcctctatgacaggctggccagtacagtcatctaccggggcaccacttttgctgagggggtggtggcattcctgatactcccccaggccaagaaagatttcttctcctctcatcctttacgcgaaccagtcaatgctacagaggacccgagctccggatattactcaaccacgatccgatatcaagccactggcttcggtaccaacgaaacagagtacctgtttgaggttgataatcttacttacgtgcagttggaaagtagattcaccccgcagtttctgctacagctcaacgagacaatttatgcgagcgggaagaggtctaataccacgggaaaactgatctggaaggtaaaccctgaaattgacactacaatcggcgagtgggcattctgggaaaccaagaaaaatcttactcggaagatacgttccgaggagctgtcatttacagccgtgagcaacggcccaaagaatattagtgggcaatctcccgctcgcacctccagcgatcctgaaacgaacaccacaaacgaggaccacaagatcatggcctcggaaaattcttccgcaatggtccaggtgcattcacagggaagaaaagctgccgttagtcacttgactaccctcgcgacaattagcacttccccacagcccccgaccacaaagaccggtcctgacaactctactcacaatacgcccgtgtacaaactggatatcagcgaggccacacaagtcggccagcatcacaggcgggcagacaacgattcaaccgctagtgacactccacccgccaccacagctgccgggcccttaaaggcagaaaataccaacacttccaagagcgccgattctctggacctcgctacaacgacctcaccacagaattatagtgagactgcgggaaacaataacacacatcaccaagataccggcgaggaaagcgcatcctctggtaaacttgggctgatcacaaatactattgccggcgtggctggattgataaccggcgggcgacgcacgagaagggaggtaatcgttaacgcccagcccaagtgtaatcctaacctacactactggaccacacaggacgaaggagcagccattggtctggcttggatcccatatttcggcccggccgcagaggggatatacactgaaggactcatgcataatcaggatggcctgatttgcggtctgcggcaactggcgaacgaaaccacacaggctttgcagctctttctgcgcgccactaccgagttaagaacgttctcgatcctgaacaggaaagcaatcgactttctactccagcgttggggcggcacatgtcacattctgggacccgattgctgtatcgaacctcatgactggaccaagaatattactgacaaaatagatcaaatcattcacgacttcgtggataagacacttccagaccagggcgataacgacaattggtggaccgggtggcggcagtggatccccgccggaatcggtgtcactggcgtgattatagctgtcatcgccttgttttgcatttgtaagttcgtgttttga

SEQ ID NO: 127 - RNA, 2046 nt, synthetic construct
  misc_feature 1..2046: note = MARV GP, Angola 2005, optimized mRNA Sequence
  source 1..2046: mol_type = other RNA
atgaaaaccacatgcctgctcatcagccttattctgatacagggcgtgaaaactttgcccatcttagagattgcctccaacatccaacctcagaatgtcgactctgtttgttcagggacgctgcagaaaaccgaagatgtgcacctaatgggattcacactcagtggtcagaaagtagctgacagcccactggaggcatccaagagatgggcctttagggcgggcgtgccgcccaaaaacgtcgaatacactgagggggaagaggctaagacctgctataatatttctgtgacagatccttcgggaaagtcacttctgttggacccacccaccaacatccgggattaccctaaatgtaagactatacatcacatccaaggccagaatccacatgcccagggtattgcactccacctgtggggcgccttctttctgtatgaccgcatcgctagcacaaccatgtaccgaggaaaagttttcacggagggcaacattgcagccatgatcgtgaataagactgtccataaaatgatatttagtcgtcagggccaagggtatagacacatgaacctcacctccacaaataagtactggacttctagcaacggaacccagacaaatgataccggttgcttcggcacgttacaggaatataactccactaagaatcagacatgtgctccctcaaagaagcctctgccacttccgaccgcccaccccgaggtgaaattgaccagtacaagcaccgacgcgacgaagctgaacaccacagatcctaattctgacgatgaggacctaactacctccgggtcaggaagcggcgagcaagagccatacacaaccagtgatgcagccaccaaacagggcctctcttccacaatgccccctaccccaagcccccagccgtcgactcctcaacagggcggaaacaacacgaatcattctcagggcgtagttacagaacccggcaagaccaacactaccgctcagccatccatgcctccccacaatacaaccactatttcaacaaacaatacgagtaaacataacctgagcaccccatccgtgcccatccaaaatgccactaactacaatacacagtctaccgcacctgagaacgaacagacaagcgctccatcaaagactacccttctgccgacggagaatcccaccacagccaagagtactaactccaccaaaagccctacaactaccgtcccaaatacgacaaacaagtattctacctcacccagtcctactccaaatagcacagcgcagcacttggtgtactttaggcggaaacgcaacattctctggagagaaggggacatgttcccctttctggacggattaatcaacgccccgatcgatttcgaccctgtgccaaataccaagactatttttgatgagtcctcttcgagcggtgcatccgctgaggaggaccaacacgcctcacccaacataagtctgaccctctcttatttccctaaagtcaatgagaacacagctcatagcggcgaaaatgagaacgattgcgacgccgaacttaggatctggtccgttcaggaggatgacctggcagccgggttgtcatggattccctttttcggaccaggcatcgagggtctatacacggctgggctgataaagaatcagaacaatctcgtgtgtcggctgcgccgacttgcgaaccagaccgcaaagagcctggaattgctcctgagagtaactacagaggaaaggaccttctctttaattaatcggcacgccatcgattttctgctagctcgttggggcggaacatgcaaagtgctcggccctgactgttgcatcgggattgaggatctgtcccgcaacatcagtgaacaaattgaccagataaagaaagacgagcagaaggagggaactggttggggccttgggggaaaatggtggaccagcgattggggcgtcttgacgaatctgggtatcctcctgcttctgtctattgccgtgttgatcgcactctcctgtatctgcagaattttcacaaagtatatagggtga

SEQ ID NO: 128 - RNA, 981 nt, synthetic construct
  misc_feature 1..981: note = EBOV VP40, Mayinga, Zaire 1976, optimized mRNA Sequence
  source 1..981: mol_type = other RNA
atgagaagggtgatcctgcccaccgcccctccagagtacatggaagctatttatccggtccggagcaactccacaatagcacgcggcgggaactctaacactggattcctcacgcccgagtcagttaatggtgacacccctagtaacccacttcgacccatcgccgatgacacaattgatcacgcgagccatactcctggctccgtgagctcggcttttatcctggaagccatggtaaatgtgatttcagggccaaaggtcttgatgaaacagatccccatatggttacctctgggagtggcagaccaaaagacctacagcttcgatagtacaaccgccgctatcatgctagcatcctatactattacacactttggcaaagccaccaacccactcgttcgtgtgaatagactgggtccggggatccccgaccatcctcttaggctgttgcggattggaaaccaggctttcctccaggagtttgtcctgccacccgtgcagctgcctcaatacttcacgtttgatctcactgccttaaagctgatcacccagccacttcccgcggcaacatggactgacgataccccgacaggctctaatggcgccttgcgccctgggatatccttccaccccaaactgcggccaattctactccctaacaagtccggaaagaaaggtaattcagctgacctgaccagtcccgaaaagatccaggccattatgacgagccttcaggattttaaaatcgtaccaatcgaccccactaagaacattatgggcatagaggttcctgagacactggtgcataaattgaccgggaagaaggtcacttctaaaaatggacaaccaatcattccggtgctcctgcccaagtatatcggcttagatcctgtggcaccaggtgacctgacaatggtcataacccaggactgcgatacgtgtcactcccccgcttcactccctgccgttattgaaaaatga

SEQ ID NO: 129 - RNA, 981 nt, synthetic construct
  misc_feature 1..981: note = EBOV VP40, Sierra Leone 2014,optimized mRNA Sequence
  source 1..981: mol_type = other RNA
atgagaagggtgatcctgcccaccgcccctccagagtacatggaagctatttatccggcacggagcaactccacaatagcccgcggcgggaactctaacactggattcctcacgcccgagtcagtcaatggtgacacccctagtaacccacttcgacccatcgcggatgacacaattgatcacgctagccatactcctggctccgtttcttcggcctttatcctggaagcaatggtgaatgtaatttcagggccaaaggtgttgatgaaacagatccccatatggttacctctgggagtcgccgaccaaaagacctacagcttcgatagtacaaccgctgcaatcatgctagcctcctatactattacacactttggcaaagctaccaacccactcgtgcgtgttaatagactgggtccggggatccccgaccatcctcttaggctgttgcggattggaaaccaggccttcctccaggagtttgtgctgccacccgtccagctgcctcaatacttcacgtttgatctcactgcgttaaagctgatcacccagccacttcccgcagccacatggactgacgataccccgacaggctctaatggcgctttgcgccctgggatatccttccaccccaaactgcggccaattctactccctaacaagtccggaaagaaaggtaattcagccgacctgaccagtcccgaaaagatccaggcaattatgacgagccttcaggattttaaaatcgtgccaatcgaccccactaagaacattatgggcatagaggtacctgagacactggttcataaattgaccgggaagaaggtgacttctaaaaatggacaaccaatcattccggtcctcctgcccaagtatatcggcttagatcctgtggctccaggtgacctgacaatggtgataacccaggactgcgatacgtgtcactcccccgcctcactccctgcggtcgttgaaaaatga

SEQ ID NO: 130 - RNA, 912 nt, synthetic construct
  misc_feature 1..912: note = MARV VP40, Angola 2005,optimized mRNA Sequence
  source 1..912: mol_type = other RNA
atggccagctcctctaactacaatacctatatgcagtacctgaacccgcctccatatgctgaccacggcgcaaatcaactcatcccggccgatcagctttcaaaccagcaggggattacacccaattacgtgggagatctgaacttggatgaccaattcaagggtaatgtctgccatgcgtttactttagaggctataatcgatattagtgcctataacgaaagaacggttaaaggcgtgcctgcatggctgccactagggatcatgagcaatttcgagtaccccctcgcccacaccgtagctgcactgcttacaggatcctatactattacccagtttacacataacggccagaagttcgtgagggtcaatcggctgggtaccgggatccctgcccacccattgcgcatgctccgagaaggaaaccaggcttttatacaaaatatggtgatcccccgtaacttctctactaatcagtttacatacaacctgaccaacctggttctctcggtgcagaaattacctgacgatgcctggcggccatcaaaggacaaactgattggcaatacgatgcatccggcggtcagcgtgcaccccaaccttcctccaatcgtattgcccactgttaagaagcaggcatacaggcaacacaaaaatcctaacaatggcccactgctagccattagtgggatcctccatcagctgcgggtggagaaggtccccgaaaagacctcccttttccgcatatctctgccggctgatatgtttagcgtgaaggagggaatgatgaaaaagagaggtgagaactcccctgtggtctatttccaggcccccgaaaattttccattgaacggcttcaataacaggcaagttgtgctcgcatacgctaatcctacactgtcagccgtatga

SEQ ID NO: 131 - RNA, 2220 nt, synthetic construct
  misc_feature 1..2220: note = EBOV NP, Zaire 1976, optimized mRNA Sequence
  source 1..2220: mol_type = other RNA
atggacagcagaccccagaagatctggatggccccttccctgaccgagtctgatatggactaccacaaaattctcacagctggcctttcagtgcaacaggggatagtcaggcagcgggttatcccagtgtatcaggtaaacaatctggaagagatttgccaattgatcattcaggcattcgaagccggagtggattttcaggagagtgcggacagcttcttactgatgctatgtctccatcacgcttaccagggtgattataagctgtttcttgaatccggcgccgtcaaatacctggaggggcatggattccgctttgaggtgaagaaacgagacggcgttaagcgtttggaagagctcctgccggcagtgagctcgggtaagaacatcaaaagaactctggccgctatgcccgaagaggagacgaccgaagcaaatgccgggcaattcctctcatttgctagcttattcctgcctaaacttgtcgtgggagagaaagcctgcttggaaaaggtacagaggcagatacaggttcacgcggagcaaggcctgatccagtatccaacagcatggcagagtgtgggccatatgatggtcatttttcggctaatgcgcactaacttcctcatcaaatttctgcttattcaccaagggatgcacatggtggccggacatgatgctaatgacgccgtgatctccaactctgtcgcacaggctagattcagcggtctgttgatagttaagaccgtgctcgatcacattctgcagaaaacagaaaggggcgtacggttacatcccctggcccgcaccgcgaaagtgaagaatgaggtcaactcctttaaagccgcactctcaagtcttgctaagcacggggagtacgcccctttcgctcgactgttgaatctaagcggagtgaacaatctggaacacggcctcttcccacagctgtctgccatcgcacttggtgttgccactgctcatgggtccacactggcgggagtgaacgtcggcgagcaatatcagcagttgagagaagcagccaccgaggctgaaaaacagctccaacagtacgccgagtcaagggagctggaccacttaggcctggatgaccaggaaaagaaaattctaatgaattttcatcagaagaagaacgagatcagcttccaacagacgaatgcaatggtgactctccggaaagaacgtctggccaaacttaccgaggctatcacagccgcaagtttgcccaaaacttctgggcactacgatgacgacgatgacattccttttccaggaccgataaacgatgacgataaccccggtcatcaggacgatgaccctaccgattcccaggacacaaccatcccagatgtagttgtggaccccgacgatggcagctatggggaataccaatcgtattctgagaatggaatgaacgcgcctgacgatctggtcctcttcgacctggatgaggacgatgaggacacgaagccagtgcccaatcgctccactaaaggcggtcagcagaagaactcacaaaaggggcagcacattgagggcagacagacacagagtaggccgatccaaaatgtccctggaccccaccggaccatacatcacgctagcgccccacttactgataacgaccgacgcaatgaaccttccggctctacaagccccagaatgctgaccccaattaacgaggaggcagatcccttggacgatgccgacgacgaaacgtcaagtctccctccactggagtccgatgacgaagagcaggatagggacgggaccagcaatcggacaccgactgtggctccccctgccccagtttaccgcgatcattctgaaaagaaggagttaccccaggacgagcagcaagatcaggaccacacccaggaagcaagaaaccaggattcagacaatacacaaagtgagcacagctttgaagagatgtataggcatatcctgcgttcccagggacctttcgatgctgtgctatactatcacatgatgaaagacgaaccagtagtcttttctacttcggatggtaaggagtacacctaccccgacagcctcgaggaagagtatccgccttggctgacagaaaaagaggccatgaacgaagagaatcggttcgtgacccttgacggccagcaattttactggccagttatgaaccataagaataagttcatggcgatcttgcagcaccatcagtga

SEQ ID NO: 132 - RNA, 2220 nt, synthetic construct
  misc_feature 1..2220: note = EBOV NP, Sierra Leone 2014, optimized mRNA Sequence
  source 1..2220: mol_type = other RNA
atggacagcagaccccagaaggtgtggatgaccccttccctgacagagtctgatatggactaccacaaaatcctcactgccggcctttcagtccaacaggggattgttaggcagcgggtgataccagtatatcaggtgaacaatctggaagagatctgccaattgattatccaggctttcgaagcaggagtcgattttcaggagagtgccgacagcttcttactgatgctatgtctccatcacgcgtaccagggtgattataagctgtttcttgaatccggcgctgtgaaatacctggaggggcatggattccgctttgaggttaagaaatgcgacggcgtgaagcgattggaagagctcctgccggccgtctcttcgggtcgtaacattaagagaacgctggcagccatgcccgaagaggaaaccacagaagctaatgcagggcaattcctctcatttgccagcttattcctgcctaaacttgtggtaggagagaaggcttgtttggaaaaagttcagaggcagatccaggtgcacgccgagcaaggcctgatacagtatccaactgcgtggcagagtgtcggccatatgatggtgatctttcggctaatgcgcaccaacttcctcattaagtttctgcttatccaccaagggatgcacatggtggcaggacatgatgccaatgacgctgtcatttccaactctgttgcccaggcaagattcagcggtctgttgatcgtgaaaacagtactcgatcacatactgcagaaaaccgaaaggggcgtgcggttacatcccctggctcgcactgccaaggtcaaaaatgaggtgaactcctttaaggcggccctctcaagtcttgcaaaacacggggagtacgctcctttcgcccgactgttgaatctaagcggagttaacaatctggaacacggcctcttcccacagctgtctgctattgcccttggtgtggcaacagcccatgggtccaccctggctggagtcaacgtgggcgagcaatatcagcagttgagagaagcggcaacggaggccgaaaagcagctccaacagtacgctgagtcaagggagctggaccacttaggcctggatgaccaggaaaagaagatcctaatgaattttcatcagaaaaagaacgagattagcttccaacagactaatgccatggtaaccctccggaaggaacgtctggcaaaacttacagaggccatcactgctgccagtttgcccaagacctctgggcactacgatgacgacgatgacatcccttttccaggaccgattaacgatgacgataaccccggtcatcaggacgatgaccctacagattcccaggacaccacgataccagatgttgtggtcgaccccgacgatggcgggtatggagaataccaaagctattcggagaatggcatgtctgcacctgacgatctggtgctcttcgacctggatgaggacgatgaggacactaaaccagtccccaaccgctccacaaagggtgggcagcagaaaaattcacaaaagggccagcacaccgagggacggcagactcagagtacaccgacccaaaacgtgacgggccctaggcggaccatccaccatgcgagcgctccccttacagataatgaccgacgcaacgaaccatccgggtctactagccctagaatgctgacccccattaatgaggaggccgatccattggacgatgcagacgacgaaacatcaagtctcccgcctctggagtccgatgacgaagagcaggatagggacggaactagcaaccggaccccaacagttgccccgcccgctcctgtgtaccgcgatcactctgaaaagaaagagttaccacaggacgagcagcaagatcaggaccatatccaggaagccagaaatcaggattcagacaacacccaacccgagcacagttttgaagagatgtataggcacatactgcgtagccagggtcctttcgatgcagtactatactatcatatgatgaaggacgaaccagtcgtgttttccacttctgatggcaaagagtacacgtaccccgactcgctcgaggaagagtatccgccttggctgacagaaaaggaggctatgaatgacgaaaaccggttcgttacccttgatgggcagcaattttactggccagtgatgaatcaccgaaacaaattcatggccattttgcagcatcaccagtga

SEQ ID NO: 133 - RNA, 2031 nt, synthetic construct
  misc_feature 1..2031: note = BDBV GP, Uganda 2007,optimized mRNA Sequence
  source 1..2031: mol_type = other RNA
atggtgaccagcggcatcctgcagctccccagagagaggttccggaaaacatcctttttcgtctgggttattatactttttcacaaagtgttccctatcccactgggggtagtgcataacaatactttgcaagtctctgacattgataagttagtgtgccgcgacaaactgtcaagtacgagccagctaaagtccgttggactcaacctggaaggtaatggcgtggccaccgatgtcccgacagctactaaacgatgggggtttcgtgcaggagtgccccctaaggtagttaactacgaggccggcgaatgggcggagaattgttataaccttgacatcaagaaagctgatggttctgaatgcctgccagaggcccccgagggggtgagaggattccctaggtgtcggtacgtccacaaggtgtcgggcaccggcccatgccccgaagggtatgcatttcataaagagggagccttctttttgtacgaccgcctcgcttcaacaattatctatagaagcaccactttcagtgaaggtgtggtcgcatttctgatactgcctgagacaaagaaagatttctttcagtccccaccgctccacgagcccgccaatatgaccacggacccttctagctactatcatactgttaccttaaactacgtggctgataatttcggcacaaacatgactaatttcctgtttcaggtagaccaccttacctacgtgcaattggaaccaaggttcacaccccagtttctggtccagctaaacgaaaccatctatacgaatgggcggcgctccaacactacaggaaccctcatttggaaggtgaatcctactgttgatacaggcgtgggtgaatgggccttctgggagaacaagaaaaactttaccaagacgctgtcaagtgaagagcttagcgtcatcttcgtgccacgagcgcaggaccccgggtctaatcaaaagaccaaggtaacaccgacttcctttgcaaacaatcagacctcaaagaaccacgaggatctggttcctgaggaccccgccagcgtggtccaggtgagagacttgcagagggagaatacagtcccaactcctcccccagataccgtgcccacaaccctcattcctgacactatggaagagcaaacgacaagtcattacgaaccaccgaacatctctcggaatcaccaggagcgtaacaataccgctcatcccgagactctggccaacaatcctccagataacaccacaccctccacccctccacaggacggagaacgcactagctcgcacacaacgccctctccgcggcctgttccaacctccactatacaccccacaaccagggagacacatattcctactaccatgacgacctcacacgatacagacagtaatcggcccaacccaatcgatattagcgaatccactgagcctggccccttaaccaatacaactcgaggcgcagctaacctgctcaccgggagccgcagaacgaggcgggaaatcacacttcgcacccaagccaagtgtaacccaaatctgcattattggactacacaggacgagggagcggccatcggtttggcatggattccgtacttcggccctgctgccgaggggatatataccgaaggaatcatgcacaaccagaatggcctaatttgcggtctgagacagctcgctaacgagactacccaagccctgcagctttttctgagggcaacaacggaattgcgtaccttcagcatcctcaatcggaaagccatagattttctgttacagcgatggggcggcacttgtcatattctgggacccgactgctgtatcgagccacacgattggacaaagaacatcaccgacaagattgaccagatcattcacgatttcatagacaaacccctacctgatcaaacagacaatgataactggtggactggctggagacagtgggtgccagctgggatcggaattaccggtgtaatcatcgcggtcattgcactcctgtgcatatgtaagtttcttttgtga

SEQ ID NO: 134 - RNA, 2031 nt, synthetic construct
  misc_feature 1..2031: note = SUDV GP, Gulu, Uganda 2007, optimized mRNA Sequence
  source 1..2031: mol_type = other RNA
atgggcgggctgagcctccttcagctgcccagagacaagttcaggaaatcctctttcttcgtgtgggtcatcattttgtttcaaaaggccttctcaatgcctttaggagttgtgaccaacagtacactggaggtaactgaaatagatcagctagtgtgcaaagaccacctcgctagcacggatcagctgaagtccgtcggtcttaatctggagggctctggggtgtcgaccgacatcccatcagcaacaaaacggtggggatttcgcagcggcgttccgcccaaggtggtcagttacgaagccggtgagtgggcggaaaactgttataatttggagattaagaaacctgatgggtccgagtgcctcccaccccctccagacggagtgcgaggcttcccccgttgtagatacgtacataaggctcagggcactgggccttgcccaggagattatgcctttcacaaagacggtgcattctttctgtacgataggctggcctctaccgttatctatcggggcgtgaacttcgctgaaggggtcattgcatttctcatcttagccaagccgaaagagacattcctgcaaagcccgcctatacgcgaagctgtgaattacaccgagaacacttcctcatattacgccacaagttaccttgagtatgaaatcgagaattttggagcgcagcatagcaccacgttgttcaagattgacaacaatactttcgtgagactggataggccacacaccccgcagtttctattccagctcaacgacacaatccatctgcaccaacagctttctaatactaccggccggctgatttggacattggatgcaaacatcaatgccgacataggtgaatgggctttttgggagaacaagaaaaatctctccgaacagctgcgcggggaggagttatcattcgaagccctgagcctcaacgaaaccgaggacgatgacgcagctagttctcgaattacgaagggaagaatctccgatagggccactcggaaatacagcgaccttgtccctaagaactcgccaggcatggttcccctgcacattccggagggcgaaacaaccttgccttctcagaactccactgaggggcgtcgcgtgggagtaaacacacaagaaaccatcacggaaaccgcggccacaatcattggtactaatggcaaccatatgcagatatcaaccatcgggattagacccagtagctcccagatcccatctagctcacctacaactgcacccagtccagaggctcaaacccccacaacccacacttccggacctagcgtgatggccacggaagagccaacaaccccgcccggctcttcacctggtccaactaccgaagctcccacactaaccactcctgagaatataacaacggccgtcaaaaccgtgctgccacaggagagtactagcaacgggctcattacatccaccgttacaggcatcctgggatctcttggcctgaggaagcggtcgcgacgccagactaataccaaagcaacggggaagtgtaaccccaatttgcattattggaccgcccaggaacaacacaacgctgcgggaatcgcatggattccgtactttggtcctggcgccgaggggatctatacagaaggactcatgcacaatcagaacgctctggtgtgcggcttaagacagctggccaatgagactacccaggcactacaactcttcctgagggccacaactgagcttcggacctacacgattttgaaccgcaaggctatagattttctgctcagaaggtggggtgggacatgtcgtatcctgggcccagactgctgtattgaaccccatgattggaccaaaaatatcactgacaagatcaaccagattatacacgatttcatcgacaaccctcttcccaatcaggataacgacgacaattggtggacaggatggcggcagtggattccagccggcatcgggattaccggaatcataatcgcaattatcgcgctgttgtgcgtctgtaaactcctgtgctga

SEQ ID NO: 135 - RNA, 2031 nt, synthetic construct
  misc_feature 1..2031: note = TAFV GP, Cote dIvoire 1994, optimized mRNA Sequence
  source 1..2031: mol_type = other RNA
atgggcgccagcgggatcctgcagctccccagagagaggttccggaaaacctcctttttcgtgtgggtcattatactttttcacaaagttttctctatccctctgggagtggtacataacaatacattgcaagtgtcagacattgataagtttgtctgccgcgacaaattaagtagcacttcccagctgaagtctgtgggtctaaacctcgaaggcaatggggttgctacggatgtgccaaccgcaacaaaacgatggggattccgtgccggcgtcccgcccaaggtggtaaactgtgaggcgggtgaatgggctgagaattgctacaacctggccatcaagaaagttgacgggtcggaatgtctccctgaggcaccagagggagtgagagattttcccaggtgccggtatgtccacaaggtgtcaggcactggcccttgtccagggggactggccttccataaagaaggtgctttcttcttgtacgaccgcctcgcaagcaccattatctatagaggcacaacctttgccgagggggtgatagctttcctgatcctgcccaaggccaggaaagatttcttccagagtcctccactccacgaaccggcgaatatgactacagacccctcctcttactatcataccacgactattaactacgtcgttgataatttcggaaccaacacaactgagtttttattccaggtggaccacctgacctatgtacaacttgaggcacggtttacacctcagttcttggtgctgctaaatgaaaccatctacagcgataaccgccgatccaatacgactggcaagctcatttggaagatcaacccaacagtcgacacctcaatgggtgagtgggccttttgggaaaataaaaagaacttcactaaaacactgagtagcgaggaactttcttttgtgcccgttcctgaaacccagaaccaggtgctggatacgaccgctacagtctccccacccatatcagcccacaatcatgcagctgaggaccacaaggaattggtgagcgaggacagtactccggtagttcaaatgcagaacattaaagggaaggataccatgcctacaactgtgaccggagtccccacaaccactccatctcctttccccatcaatgccagaaacacggaccatacaaaatcctttattggcctcgaaggcccacaggaggatcacagcaccactcagcccgcgaaaaccacatcgcaacctaccaattctactgaatccacaacgctgaacccaacctcagagccgagtagcagggggactggaccctcctctcctacagtgccaaataccacagagagccacgccgaattaggtaagactacccccacgaccctgcctgagcagcatacagcagcttcagccatcccacgggctgtccaccccgacgaactcagtggcccggggttccttactaacaccatccgtggagtgacaaatctgttgactggctcccgcagaaaaaggcgggatgttacccctaacacgcagccaaagtgcaatcccaacctacattactggacagccctggacgagggtgcagccattgggctcgcttggataccttattttggccccgcggcagaaggaatctacaccgagggcattatggagaatcaaaacgggctgatctgtggacttcgacagctggccaatgaaactacacaggctttgcagctcttcctgcgcgccaccactgagttaagaacctttagcatactgaacaggaaagcaattgatttcctactccaacggtggggtggcacatgccacatcctggggccagactgttgcatcgaacctcaggattggacgaagaacattaccgacaaaatcgatcagattatacatgacttcgtggacaataaccttcccaatcagaacgatggatctaattggtggactggctggaagcaatgggtaccagccggtatcgggattacaggcgtcatcatcgctattatagccttgctgtgtatctgcaagtttatgctctga

SEQ ID NO: 136 - RNA, 981 nt, synthetic construct
  misc_feature 1..981: note = BDBV VP40, Uganda 2007, optimized mRNA Sequence
  source 1..981: mol_type = other RNA
atgagaagggccatcctgcccaccgctcctccagagtacattgaagcagtgtatccgatgcggacagtcagcacttccataaactctacggcctcaggccccaatttccctgcgccagacgttatgatgagtgatacccccagcaactccctccgccctatcgctgacgataatattgaccacccatctcatacacccacttcggtgtcaagcgcctttatccttgaggcaatggtaaacgtgattagtgggcctaaggtcctgatgaaacagatcccaatatggttgccgttaggagtggccgatcaaaagacctactccttcgactctacaaccgctgcaatcatgctggccagctatactattacacactttggtaaaacctccaatcccctagttcgaatcaaccgtctcggccctgggattccagatcatcccctgagacttctgaggatcggaaatcaggctttcttgcaggaatttgtgctccctccagtccagctgccccaatacttcacgtttgacctgactgccctcaagttaataacccagccgctgcctgcggcaacatggactgatgacacccccacaggcccaaccggtattcttcggcctgggatctcattccaccccaaattgcgcccaattctgctacccggaaagacgggcaagagaggcagtagctctgatctcacttcccctgacaaaatccaggccatcatgaactttctgcaggatcttaagctggtgccaattgacccggctaaaaatataatggggatcgaggtacccgaattgctcgttcataggctgacaggaaagaaaattaccactaagaacggtcaacctatcataccaattttactgcccaagtatatcggcatggaccctatctcacagggggatctcacaatggtgattacccaggactgcgatacgtgtcacagcccagccagtctcccgccggtgtctgagaaatga

SEQ ID NO: 137 - RNA, 981 nt, synthetic construct
  misc_feature 1..981: note = SUDV VP40, Gulu, Uganda 2000, optimized mRNA Sequence
  source 1..981: mol_type = other RNA
atgagaagggtgaccgtccccacagcccctccagcttacgcagacatcggctatccgatgagcatgctgcccattaagtcctctcgggccgtttcagggatacagcaaaaacaggaggtgctccctggaatggatactccaagtaacagcatgcgccccgtagcggacgataatatcgaccacacgtcccatacccctaacggtgtggcttctgccttcattcttgaagcaacagtcaatgtgatctcgggcccaaaggttctgatgaaacagattcccatctggttgcctttagggatagccgatcagaaaacttactcatttgacagcaccacagctgcaatcatgctggccagttataccattactcacttcggaaaagctaacaatccactcgtgcgagtcaaccgtctcggccaaggtatcccggatcatcccctgagacttctgaggatggggaatcaggcctttttgcaggagttcgtgctccctccagtacagctgccccaatactttacattcgacctgaccgcgctcaagttagttacgcagcctctgccagcagccacttggaccgatgaaacaccctccaacctttctggagctttgcggccgggcctgagctttcaccctaagctacgccccgtgctcctgccaggcaaaactgggaagaaaggacatgtctccgaccttaccgcccctgataagattcagacaatcgtgaatctgatgcaggacttcaaaatagtgcccattgatccagcaaagtcaatcattggtatcgaggtccccgaattgctcgttcacaagctgaccggcaaaaagatgagtcaaaagaacgggcagcctatcattccagtgttactgccgaagtatataggactcgaccccatcagccctggcgaccttacgatggtaattactccagattacgacgattgccactctcccgcttcctgttcatatctgagcgagaaatga

SEQ ID NO: 138 - RNA, 981 nt, synthetic construct
  misc_feature 1..981: note = TAFV VP40, Cote dIvoire 1994, optimized mRNA Sequence
  source 1..981: mol_type = other RNA
atgagaaggatcattctgcccaccgcccctccagagtacatggaagctgtgtatccgatgcggacaatgaacagcggcgcagacaatactgcctccgggcccaactacacgaccacaggagtcatgactaatgataccccttctaactcactccgcccagttgcggacgataatatagaccaccccagtcatacacctaacagcgtggcttccgccttcatccttgaggcaatggtaaatgtgatttctggtccaaaggtcctgatgaaacagatccccatttggttgcctttaggcgtgtcggatcaaaagacctattcatttgacagcactacagccgctatcatgctggcaagttacaccataacgcacttcgggaaaacttccaacccactcgttcgaatcaatcgtctcggaccgggcattcccgatcatcctctgagacttctgaggatcggtaaccaggcctttttgcaggagttcgtgctcccacccgtccagctgcctcaatattttaccttcgacctgacagctctcaagttaattactcagccactgcccgccgcgacctggacagatgaaaccccggcagtgtctacggggactcttcggcctggaatcagctttcaccccaaattgcgcccaatactgctacctggcagagccggcaagaaggggtccaattcagacctcacaagtcccgataaaattcaggctatcatgaacttcctgcaggaccttaagattgtaccaatcgatcccaccaaaaatatcatgggaattgaagttcctgagctgttggtgcataggctcactggtaagaaaacaaccacgaagaacggccaaccaataatcccgattctgttacccaagtacatcgggctggaccctctcagccagggagatcttaccatggtcataacacaggattgcgactcttgtcactccccagcctcactgccccctgtgaatgagaaatga

SEQ ID NO: 139 - RNA, 2088 nt, synthetic construct
  misc_feature 1..2088: note = MARV NP, Angola 2005, optimized mRNA Sequence
  source 1..2088: mol_type = other RNA
atggacctgcacagcctccttgagctgggcaccaagcccacagcccctcatgtgagaaacaaaaaggtcatcttgttcgatactaatcaccaggtttccatttgcaaccaaataatcgacgctattaattctgggatcgatttaggagatctgctagaaggtggcctcctgacgctttgtgtggagcattactataactcagataaagacaagtttaataccagtccaattgcaaaatacctgagggatgccgggtatgagttcgacgtaatcaagaacgcggatgctacacggtttttggacgtgataccgaatgagccccactacagccctctcatcctggccctgaaaactctcgaatccaccgagtctcagcgcggacgaattggcttattcctgtcgttttgctcacttttcttgccaaaactggtcgtgggtgatcgtgcaagcatcgagaaggccctaagacaggttacagtgcatcaggaacaagggattgtcacctatcccaaccactggctcactacaggacacatgaaagtgatctttggcatactgaggagttccttcattcttaagtttgtactgatccatcagggcgttaatttggtgaccgggcacgacgcttacgattctattatcagcaactccgtcggacagacgcggttctcaggcctcctgatcgtgaaaactgtgttagagtttattctgcagaaaaccgacagtggcgtcacactccatcctcttgttcgcactagcaaggtgaaaaatgaagtagcatctttcaagcaagccctgtccaacttggctagacacggggagtatgccccatttgcgagggtgctaaatctgtcaggaataaacaatctcgagcacggcctgtacccccagcttagcgcaatcgccctgggtgtcgctaccgcccatgggagtacattggcaggagtgaacgttggcgaacagtaccagcaactccgggaggctgcccacgacgcggaagtgaaactgcagcgccgacatgagcaccaggaaattcaagccatcgcagaggatgacgaggaaagaaagatattagagcagttccatctgcagaaaaccgaaattacgcactctcagactctagctgtcctctcccaaaagagggagaaactggcccggcttgctgccgaaatcgagaacaatatcgtggaggatcagggcttcaagcagagccagaaccgtgtatcgcaatctttcttgaatgaccctacaccagttgaagtgaccgtccaggcacgcccgattaacagacccactgccctgcctccacccgtggattccaagatcgagcacgaatcaacagaggacagtagctcctctagctcattcgtcgatctcaatgacccttttgctctgcttaacgaagatgaggacaccctggatgacagtgtgatgattccatccacgaccagcagggagttccaggggatacccgaaccgcctcggcagtctcaagacatcgataattcacagggaaaacaggaggacgaaagtacaaacttgattaagaaaccctttctccgatatcaggagctgccacctgttcaagaggatgacgaaagcgagtacactaccgattcccaggaatctatcgaccagcccggttcggataatgagcaaggcgtggacttaccaccccctccactgtatgcgcaggaaaagcgccaggatccgatccagcatcccgcagtaagctcccaagaccctttcgggtcaattggagatgtcaacggcgacatactagagccaatcagaagtccctctagcccttccgccccacaggaggatacaagggctcgggaagcctacgagctctcacccgactttactaattatgaggacaaccagcagaattggccgcaacgcgtggttaccaaaaagggtagaacattcctgtaccctaacgatcttttgcagaccaatccacccgagagcctgattactgcactcgtggaagagtatcagaaccctgtctctgccaaggagctgcaggctgactggcccgatatgtcctttgacgaaaggcgtcacgtggccatgaacttatga

SEQ ID NO: 140 - RNA, 2220 nt, synthetic construct
  misc_feature 1..2220: note = BDBV NP, Uganda 2007, optimized mRNA Sequence
  source 1..2220: mol_type = other RNA
atggaccccagacctatcaggacctggatgatgcacaacacaagcgaggtggaagccgattaccataagattctgactgctggcctctccgtccagcaagggatagttcggcagcgcatcattccagtgtatcagatctctaatcttgaggaagtatgccagctgattatccaagcattcgaggccggagtggactttcaggattcagcggacagtttcttgttaatgctgtgtctacaccatgcttaccagggtgattataaacagtttctcgaaagcaacgccgtcaagtacctggagggccacgggttccgatttgagatgaaaaagaaagaaggagtgaagcgtcttgaggaactgttgccggcagcctcctctggcaagaatataaaaagaacgctcgctgcaatgcccgaggaggaaaccacagaggccaacgctggtcaattcctgtcgtttgcctcactgttcctccctaagttagttgtgggggaaaaagcgtgcctggagaaggtccagaggcagatccaggtgcatgcagaacaaggacttattcagtatccaactagctggcagagtgtaggccacatgatggttatctttcggttgatgcgcaccaatttcctgattaaatttctactcatccaccaaggcatgcatatggtggccgggcacgacgctaacgatgccgtcatagcaaattccgtggctcaggccagattctctggactgcttattgtgaaaacagtcctggaccatatcttgcagaaaaccgagcacggtgttaggctccaccccctggcgcggactgccaaagtgaagaacgaggtaagctcctttaaagcagctttagcctcactggctcagcatggcgaatacgcccctttcgcacgcctccttaatctgagtggggtgaacaatttggagcacggactattcccacaactgagcgccattgctctcggcgtcgcgacagcacatggttctaccctggccggggtgaacgttggagaacagtatcagcagcttcgagaggctgccacggaagcagagaagcaactgcagaaatacgccgagtccagagaattggatcacctcggcctggacgatcaggagaagaaaatcttaaaggactttcatcagaagaaaaatgaaatctcattccaacagactaccgctatggtgacactgaggaaggagcggctagccaaactcactgaagcaattaccagcacaagtatactgaaaaccggccgtcgctacgatgacgacaacgatatcccctttcctgggccaattaatgacaacgagaactctggacagaatgatgacgatccgacggactcccaggatactacaatccccgacgtcataattgatcctaacgacggtggctataataactacagcgattatgcgaatgacgctgcctcggcaccagacgatcttgtgttgttcgacctggaggatgaggacgatgccgacaaccccgctcaaaatacccctgagaaaaacgatagaccagccactacaaagctcaggaatgggcaggaccaggatggaaaccaaggcgaaaccgcatctccccgggtagctccgaatcagtaccgagacaagcctatgccccaggttcaggatcgctccgagaaccacgaccaaacgctgcagacccagtcaagagtgcttacaccaatcagtgaggaagccgatcctagcgaccacaatgacggtgataacgagtccatcccgccactggaatctgacgatgaggggagcactgacaccacagcggccgaaactaaacccgctaccgcacctccagccccggtctataggtcaattagtgtggatgactccgtccccagcgagaatatccctgctcagtctaaccaaacaaacaatgaggataacgtgcggaataacgcccagtcagaacagagtattgcagagatgtaccagcatatattgaaaacccaaggcccatttgacgcgatcctctattaccacatgatgaaagaagagcccattatcttcagcacttccgatggaaaggaatacacgtatcctgactctctggaggatgagtacccaccctggttatcggaaaaagaggccatgaatgaggacaaccgctttatcacaatggatggccagcagttctattggccggttatgaatcatagaaacaagtttatggctattctgcaacaccacaggtga

SEQ ID NO: 141 - RNA, 2217 nt, synthetic construct
  misc_feature 1..2217: note = SUDV NP, Gulu, Uganda 2000,optimized mRNA Sequence
  source 1..2217: mol_type = other RNA
atggacaagagagtgaggggcagctgggccctgggcggacagtccgaggtcgatctcgactaccacaaaatccttaccgctggtctgtctgttcaacagggcattgtgcggcagcgcgtaatacccgtgtatgtcgtgtcagatttggaagggatctgccagcatattatccaagcattcgaggccggagttgactttcaggataacgcggacagtttcttactgctactctgtctgcaccatgcttaccagggcgatcaccgactttttctgaagagcgacgccgtgcagtatttggaaggtcatgggttccgttttgaggtcagagaaaaagagaatgtgcacaggctcgatgagctgctgcctaacgtaacaggaggcaagaatctccggcgcactttagcagccatgccagaagaggaaacgaccgaggctaacgcaggccaattcctgtcctttgcctctcttttcttgccgaaactggttgtgggggagaaggcttgcctagaaaaggtccagagacagattcaggtgcacgccgagcaaggactcatccagtaccccacatcgtggcagtcagtgggtcatatgatggtcatatttaggctgatgcggactaatttccttatcaaatttctgttgattcaccaaggcatgcatatggttgcggggcacgacgcaaacgataccgtgatcagcaatagtgtagcccaggctcgcttctccggactcctgattgtgaaaacagtcttagaccacatcctgcagaaaaccgatctcggcgtgcgacttcatcctctggccagaactgcaaaggttaaaaacgaagtgtctagctttaaggctgccttgggttccctagcgaagcacggggagtatgccccattcgcaaggctgctcaatctgtcaggagtcaacaatcttgaacatggcctgtacccccagttgagtgctatagccctcggcgtggctacagcccacgggagcaccctggcaggagtaaacgttggtgagcaatatcagcagttacgggaggccgctacggaagcggagaaacagctgcaacagtacgcagaaactcgtgagctagacaatctcggcctggacgaacaggagaagaaaattcttatgtctttccatcagaagaaaaacgagatctcctttcaacagaccaatgccatggtgacattgcgcaaggaaagactggctaaactcactgaggccattaccacagcatcaaagatcaaggtcggggataggtaccctgacgataacgacatcccattccccggacctatttatgatgaaacccacccaaacccgagcgacgataatcccgacgatagtcgggacacgactatacctggcggtgtggtcgatccatacgacgacgagtctaacaattatcccgattacgaggactccgccgaggggacaaccggcgatctggacctttttaacctggatgacgatgacgatgacagccagcctggaccacccgatcgaggccagtcgaaagagcgcgctgccagaactcacgggttgcaagacccgacactcgatggagcaaagaaagtgcctgaactgacccccggttctcatcagccaggcaatttacacatcacgaagcctgggtccaacaccaatcagccccaaggaaacatgtcaagtacactgcagagcatgactccaattcaggaggaatccgagcccgacgaccagaaagatgacgatgacgagtctctaaccagcctcgattcagaaggcgacgaggatgttgaaagtgtgtccggtgagaataaccctacagtagcgccaccggctcccgtctataaggacactggggtggataccaatcaacagaacggccctagcaatgccgttgacggacagggctctgaatcagaggcactgccaatcaaccccgagaaggggagtgcccttgaagagacatactatcatttgctgaaaacccagggacctttcgaagctataaattactaccacctcatgagcgatgagccaattgccttttccactgaatctggtaaggagtatatcttccccgactcgctggaggaagcatacccgccttggttaagcgagaaagaagctctggagaaggaaaacaggtatcttgtgatcgatggccaacagtttttgtggccagtcatgtccctccaggacaaattcctggccgtgctacagcacgactga

SEQ ID NO: 142 - RNA, 2220 nt, synthetic construct
  misc_feature 1..2220: note = TAFV NP, Cote dIvoire 1994,optimized mRNA Sequence
  source 1..2220: mol_type = other RNA
atggagagcagagcccacaaggcttggatgacccatacagcatccggcttcgaaactgactaccacaaaatcctgacggccgggctctctgtgcagcaaggaattgtcaggcagcgggttatacaggtgcatcaggtaaccaaccttgaggaaatctgccaactgattatccaggcgtttgaggctggtgtggatttccaggaatcagccgacagtttcttgttaatgctgtgtctacaccatgcatatcagggcgattacaagcaattcctcgagagcaatgccgtcaaatatctggaggggcacggatttcgcttcgaagtgcgaaagaaagagggcgttaagcgtcttgaagagctgttgcccgctgcatcctctggtaagtcgattagaaggacactcgccgctatgcctgaggaagagactaccgaagccaacgcggggcagtttctgtcattcgcaagcctgtttctcccaaaattagtggtcggagagaaggcctgcctggaaaaagtgcagcggcagatccaagtacacagtgagcagggccttatacagtacccgacagcttggcaatccgttggccatatgatggtgatcttccgcttgatgagaaccaattttctgattaagttcctactcatccaccaggggatgcatatggtcgccggacacgacgcaaacgatgctgtgattgccaattctgtggcgcaggccaggtttagcggtctgcttatcgtcaaaactgttctggaccacatattgcagaaaacagagcatggcgtgcggctccaccccctggcacgcaccgctaaggtaaagaacgaagtgaactccttcaaggccgctttatcaagtctggcccaacatggggagtatgcacctttcgcccgactccttaacctgagcggagtcaataacttggaacacggcctatttccacagctgtctgctattgcgctcggtgtggcaacggcccatgggtccactctggctggagttaatgtgggcgagcagtaccagcaacttagagaagccgcaaccgaggccgagaaacagctgcagaagtatgctgaatcaagggagttggatcacctcggcctggacgatcaggaaaagaagatcttaaaagacttccaccaaaagaagaacgagattagctttcagcagacaactgccatggtcaccctgcggaaagaacgtctagcaaagctcacagaggcgatcaccagtacgtctctgcttaaaactgggaagcagtacgatgacgataatgacatccccttccctggaccaattaacgacaatgagaactccgaacaacaggatgacgatcccacagacagccaggataccactatacctgacatcattgtggatccagacgatggtcgctacaacaattatggcgactacccgtcggagacagctaacgcccccgaagatttggtactgtttgacctcgaggacggggatgaggacgatcatagaccttcttcctcaagtgaaaataacaataaacacagcctgaccggaacggactccaacaagacctctaattggaacaggaatccaacaaacatgcccaagaaagatagcactcaaaataacgacaatcctgcacagcgggcccaggagtatgctcgagataacatccaggacaccccaacaccccatcgcgcccttactccgatatcagaagaaaccggcagtaatggtcacaacgaagatgacattgattccatccctcccctggagagcgacgaggaaaacaatacagaaaccactatcacgacaaccaagaacactaccgcaccacccgctcccgtttacagatctaattcagaaaaagagccattgccccaagaaaagagtcagaaacagcctaaccaggtgagcgggtccgagaatacagataacaagccacactctgagcaatcggtcgaagagatgtataggcatattctccagacccagggcccgttcgacgccatcctgtactattacatgatgactgaagagcccattgtgtttagcacatccgacggaaaggaatacgtctatcctgattcattagagggcgagcacccaccctggctgagtgaaaaagaggcgctaaatgaggacaaccggttcataacgatggatgaccagcaattttactggcctgtgatgaatcatcgcaacaagttcatggccatcctccagcaccataaatga

SEQ ID NO: 143 - RNA, 2031 nt, synthetic construct
  misc_feature 1..2031: note = EBOV GP, Mayinga, Zaire 1976, optimized mRNA Sequence
  source 1..2031: mol_type = other RNA
atgggcgtgaccggcatcctgcagctgccccgcgaccgcttcaagcgcaccagcttcttcctgtgggtgatcatcctgttccagcgcaccttcagcatccccctgggcgtgatccacaacagcaccctgcaggtgagcgacgtggacaagctggtgtgccgcgacaagctgagcagcaccaaccagctgcgcagcgtgggcctgaacctggagggcaacggcgtggccaccgacgtgcccagcgccaccaagcgctggggcttccgcagcggcgtgccccccaaggtggtgaactacgaggccggcgagtgggccgagaactgctacaacctggagatcaagaagcccgacggcagcgagtgcctgcccgccgcccccgacggcatccgcggcttcccccgctgccgctacgtgcacaaggtgagcggcaccggcccctgcgccggcgacttcgccttccacaaggagggcgccttcttcctgtacgaccgcctggccagcaccgtgatctaccgcggcaccaccttcgccgagggcgtggtggccttcctgatcctgccccaggccaagaaggacttcttcagcagccaccccctgcgcgagcccgtgaacgccaccgaggaccccagcagcggctactacagcaccaccatccgctaccaggccaccggcttcggcaccaacgagaccgagtacctgttcgaggtggacaacctgacctacgtgcagctggagagccgcttcaccccccagttcctgctgcagctgaacgagaccatctacaccagcggcaagcgcagcaacaccaccggcaagctgatctggaaggtgaaccccgagatcgacaccaccatcggcgagtgggccttctgggagaccaagaagaacctgacccgcaagatccgcagcgaggagctgagcttcaccgtggtgagcaacggcgccaagaacatcagcggccagagccccgcccgcaccagcagcgaccccggcaccaacaccaccaccgaggaccacaagatcatggccagcgagaacagcagcgccatggtgcaggtgcacagccagggccgcgaggccgccgtgagccacctgaccaccctggccaccatcagcaccagcccccagagcctgaccaccaagcccggccccgacaacagcacccacaacacccccgtgtacaagctggacatcagcgaggccacccaggtggagcagcaccaccgccgcaccgacaacgacagcaccgccagcgacacccccagcgccaccaccgccgccggcccccccaaggccgagaacaccaacaccagcaagagcaccgacttcctggaccccgccaccaccaccagcccccagaaccacagcgagaccgccggcaacaacaacacccaccaccaggacaccggcgaggagagcgccagcagcggcaagctgggcctgatcaccaacaccatcgccggcgtggccggcctgatcaccggcggccgccgcacccgccgcgaggccatcgtgaacgcccagcccaagtgcaaccccaacctgcactactggaccacccaggacgagggcgccgccatcggcctggcctggatcccctacttcggccccgccgccgagggcatctacatcgagggcctgatgcacaaccaggacggcctgatctgcggcctgcgccagctggccaacgagaccacccaggccctgcagctgttcctgcgcgccaccaccgagctgcgcaccttcagcatcctgaaccgcaaggccatcgacttcctgctgcagcgctggggcggcacctgccacatcctgggccccgactgctgcatcgagccccacgactggaccaagaacatcaccgacaagatcgaccagatcatccacgacttcgtggacaagaccctgcccgaccagggcgacaacgacaactggtggaccggctggcgccagtggatccccgccggcatcggcgtgaccggcgtgatcatcgccgtgatcgccctgttctgcatctgcaagttcgtgttctaa

SEQ ID NO: 144 - RNA, 2031 nt, synthetic construct
  misc_feature 1..2031: note = EBOV GP, Sierra Leone 2014, optimized mRNA Sequence
  source 1..2031: mol_type = other RNA
atgggcgtgaccggcatcctgcagctgccccgcgaccgcttcaagcgcaccagcttcttcctgtgggtgatcatcctgttccagcgcaccttcagcatccccctgggcgtgatccacaacagcaccctgcaggtgagcgacgtggacaagctggtgtgccgcgacaagctgagcagcaccaaccagctgcgcagcgtgggcctgaacctggagggcaacggcgtggccaccgacgtgcccagcgtgaccaagcgctggggcttccgcagcggcgtgccccccaaggtggtgaactacgaggccggcgagtgggccgagaactgctacaacctggagatcaagaagcccgacggcagcgagtgcctgcccgccgcccccgacggcatccgcggcttcccccgctgccgctacgtgcacaaggtgagcggcaccggcccctgcgccggcgacttcgccttccacaaggagggcgccttcttcctgtacgaccgcctggccagcaccgtgatctaccgcggcaccaccttcgccgagggcgtggtggccttcctgatcctgccccaggccaagaaggacttcttcagcagccaccccctgcgcgagcccgtgaacgccaccgaggaccccagcagcggctactacagcaccaccatccgctaccaggccaccggcttcggcaccaacgagaccgagtacctgttcgaggtggacaacctgacctacgtgcagctggagagccgcttcaccccccagttcctgctgcagctgaacgagaccatctacgccagcggcaagcgcagcaacaccaccggcaagctgatctggaaggtgaaccccgagatcgacaccaccatcggcgagtgggccttctgggagaccaagaagaacctgacccgcaagatccgcagcgaggagctgagcttcaccgccgtgagcaacggccccaagaacatcagcggccagagccccgcccgcaccagcagcgaccccgagaccaacaccaccaacgaggaccacaagatcatggccagcgagaacagcagcgccatggtgcaggtgcacagccagggccgcaaggccgccgtgagccacctgaccaccctggccaccatcagcaccagcccccagccccccaccaccaagaccggccccgacaacagcacccacaacacccccgtgtacaagctggacatcagcgaggccacccaggtgggccagcaccaccgccgcgccgacaacgacagcaccgccagcgacaccccccccgccaccaccgccgccggccccctgaaggccgagaacaccaacaccagcaagagcgccgacagcctggacctggccaccaccaccagcccccagaactacagcgagaccgccggcaacaacaacacccaccaccaggacaccggcgaggagagcgccagcagcggcaagctgggcctgatcaccaacaccatcgccggcgtggccggcctgatcaccggcggccgccgcacccgccgcgaggtgatcgtgaacgcccagcccaagtgcaaccccaacctgcactactggaccacccaggacgagggcgccgccatcggcctggcctggatcccctacttcggccccgccgccgagggcatctacaccgagggcctgatgcacaaccaggacggcctgatctgcggcctgcgccagctggccaacgagaccacccaggccctgcagctgttcctgcgcgccaccaccgagctgcgcaccttcagcatcctgaaccgcaaggccatcgacttcctgctgcagcgctggggcggcacctgccacatcctgggccccgactgctgcatcgagccccacgactggaccaagaacatcaccgacaagatcgaccagatcatccacgacttcgtggacaagaccctgcccgaccagggcgacaacgacaactggtggaccggctggcgccagtggatccccgccggcatcggcgtgaccggcgtgatcatcgccgtgatcgccctgttctgcatctgcaagttcgtgttctaa

SEQ ID NO: 145 - RNA, 2046 nt, synthetic construct
  misc_feature 1..2046: note = MARV GP, Angola 2005, optimized mRNA Sequence
  source 1..2046: mol_type = other RNA
atgaagaccacctgcctgctgatcagcctgatcctgatccagggcgtgaagaccctgcccatcctggagatcgccagcaacatccagccccagaacgtggacagcgtgtgcagcggcaccctgcagaagaccgaggacgtgcacctgatgggcttcaccctgagcggccagaaggtggccgacagccccctggaggccagcaagcgctgggccttccgcgccggcgtgccccccaagaacgtggagtacaccgagggcgaggaggccaagacctgctacaacatcagcgtgaccgaccccagcggcaagagcctgctgctggacccccccaccaacatccgcgactaccccaagtgcaagaccatccaccacatccagggccagaacccccacgcccagggcatcgccctgcacctgtggggcgccttcttcctgtacgaccgcatcgccagcaccaccatgtaccgcggcaaggtgttcaccgagggcaacatcgccgccatgatcgtgaacaagaccgtgcacaagatgatcttcagccgccagggccagggctaccgccacatgaacctgaccagcaccaacaagtactggaccagcagcaacggcacccagaccaacgacaccggctgcttcggcaccctgcaggagtacaacagcaccaagaaccagacctgcgcccccagcaagaagcccctgcccctgcccaccgcccaccccgaggtgaagctgaccagcaccagcaccgacgccaccaagctgaacaccaccgaccccaacagcgacgacgaggacctgaccaccagcggcagcggcagcggcgagcaggagccctacaccaccagcgacgccgccaccaagcagggcctgagcagcaccatgccccccacccccagcccccagcccagcaccccccagcagggcggcaacaacaccaaccacagccagggcgtggtgaccgagcccggcaagaccaacaccaccgcccagcccagcatgcccccccacaacaccaccaccatcagcaccaacaacaccagcaagcacaacctgagcacccccagcgtgcccatccagaacgccaccaactacaacacccagagcaccgcccccgagaacgagcagaccagcgcccccagcaagaccaccctgctgcccaccgagaaccccaccaccgccaagagcaccaacagcaccaagagccccaccaccaccgtgcccaacaccaccaacaagtacagcaccagccccagccccacccccaacagcaccgcccagcacctggtgtacttccgccgcaagcgcaacatcctgtggcgcgagggcgacatgttccccttcctggacggcctgatcaacgcccccatcgacttcgaccccgtgcccaacaccaagaccatcttcgacgagagcagcagcagcggcgccagcgccgaggaggaccagcacgccagccccaacatcagcctgaccctgagctacttccccaaggtgaacgagaacaccgcccacagcggcgagaacgagaacgactgcgacgccgagctgcgcatctggagcgtgcaggaggacgacctggccgccggcctgagctggatccccttcttcggccccggcatcgagggcctgtacaccgccggcctgatcaagaaccagaacaacctggtgtgccgcctgcgccgcctggccaaccagaccgccaagagcctggagctgctgctgcgcgtgaccaccgaggagcgcaccttcagcctgatcaaccgccacgccatcgacttcctgctggcccgctggggcggcacctgcaaggtgctgggccccgactgctgcatcggcatcgaggacctgagccgcaacatcagcgagcagatcgaccagatcaagaaggacgagcagaaggagggcaccggctggggcctgggcggcaagtggtggaccagcgactggggcgtgctgaccaacctgggcatcctgctgctgctgagcatcgccgtgctgatcgccctgagctgcatctgccgcatcttcaccaagtacatcggctaa

SEQ ID NO: 146 - RNA, 981 nt, synthetic construct
  misc_feature 1..981: note = EBOV VP40, Mayinga, Zaire 1976, optimized mRNA Sequence
  source 1..981: mol_type = other RNA
atgcgccgcgtgatcctgcccaccgccccccccgagtacatggaggccatctaccccgtgcgcagcaacagcaccatcgcccgcggcggcaacagcaacaccggcttcctgacccccgagagcgtgaacggcgacacccccagcaaccccctgcgccccatcgccgacgacaccatcgaccacgccagccacacccccggcagcgtgagcagcgccttcatcctggaggccatggtgaacgtgatcagcggccccaaggtgctgatgaagcagatccccatctggctgcccctgggcgtggccgaccagaagacctacagcttcgacagcaccaccgccgccatcatgctggccagctacaccatcacccacttcggcaaggccaccaaccccctggtgcgcgtgaaccgcctgggccccggcatccccgaccaccccctgcgcctgctgcgcatcggcaaccaggccttcctgcaggagttcgtgctgccccccgtgcagctgccccagtacttcaccttcgacctgaccgccctgaagctgatcacccagcccctgcccgccgccacctggaccgacgacacccccaccggcagcaacggcgccctgcgccccggcatcagcttccaccccaagctgcgccccatcctgctgcccaacaagagcggcaagaagggcaacagcgccgacctgaccagccccgagaagatccaggccatcatgaccagcctgcaggacttcaagatcgtgcccatcgaccccaccaagaacatcatgggcatcgaggtgcccgagaccctggtgcacaagctgaccggcaagaaggtgaccagcaagaacggccagcccatcatccccgtgctgctgcccaagtacatcggcctggaccccgtggcccccggcgacctgaccatggtgatcacccaggactgcgacacctgccacagccccgccagcctgcccgccgtgatcgagaagtaa

SEQ ID NO: 147 - RNA, 981 nt, synthetic construct
  misc_feature 1..981: note = EBOV VP40, Sierra Leone 2014,optimized mRNA Sequence
  source 1..981: mol_type = other RNA
atgcgccgcgtgatcctgcccaccgccccccccgagtacatggaggccatctaccccgcccgcagcaacagcaccatcgcccgcggcggcaacagcaacaccggcttcctgacccccgagagcgtgaacggcgacacccccagcaaccccctgcgccccatcgccgacgacaccatcgaccacgccagccacacccccggcagcgtgagcagcgccttcatcctggaggccatggtgaacgtgatcagcggccccaaggtgctgatgaagcagatccccatctggctgcccctgggcgtggccgaccagaagacctacagcttcgacagcaccaccgccgccatcatgctggccagctacaccatcacccacttcggcaaggccaccaaccccctggtgcgcgtgaaccgcctgggccccggcatccccgaccaccccctgcgcctgctgcgcatcggcaaccaggccttcctgcaggagttcgtgctgccccccgtgcagctgccccagtacttcaccttcgacctgaccgccctgaagctgatcacccagcccctgcccgccgccacctggaccgacgacacccccaccggcagcaacggcgccctgcgccccggcatcagcttccaccccaagctgcgccccatcctgctgcccaacaagagcggcaagaagggcaacagcgccgacctgaccagccccgagaagatccaggccatcatgaccagcctgcaggacttcaagatcgtgcccatcgaccccaccaagaacatcatgggcatcgaggtgcccgagaccctggtgcacaagctgaccggcaagaaggtgaccagcaagaacggccagcccatcatccccgtgctgctgcccaagtacatcggcctggaccccgtggcccccggcgacctgaccatggtgatcacccaggactgcgacacctgccacagccccgccagcctgcccgccgtggtggagaagtaa

SEQ ID NO: 148 - RNA, 912 nt, synthetic construct
  misc_feature 1..912: note = MARV VP40, Angola 2005,optimized mRNA Sequence
  source 1..912: mol_type = other RNA
atggccagcagcagcaactacaacacctacatgcagtacctgaacccccccccctacgccgaccacggcgccaaccagctgatccccgccgaccagctgagcaaccagcagggcatcacccccaactacgtgggcgacctgaacctggacgaccagttcaagggcaacgtgtgccacgccttcaccctggaggccatcatcgacatcagcgcctacaacgagcgcaccgtgaagggcgtgcccgcctggctgcccctgggcatcatgagcaacttcgagtaccccctggcccacaccgtggccgccctgctgaccggcagctacaccatcacccagttcacccacaacggccagaagttcgtgcgcgtgaaccgcctgggcaccggcatccccgcccaccccctgcgcatgctgcgcgagggcaaccaggccttcatccagaacatggtgatcccccgcaacttcagcaccaaccagttcacctacaacctgaccaacctggtgctgagcgtgcagaagctgcccgacgacgcctggcgccccagcaaggacaagctgatcggcaacaccatgcaccccgccgtgagcgtgcaccccaacctgccccccatcgtgctgcccaccgtgaagaagcaggcctaccgccagcacaagaaccccaacaacggccccctgctggccatcagcggcatcctgcaccagctgcgcgtggagaaggtgcccgagaagaccagcctgttccgcatcagcctgcccgccgacatgttcagcgtgaaggagggcatgatgaagaagcgcggcgagaacagccccgtggtgtacttccaggcccccgagaacttccccctgaacggcttcaacaaccgccaggtggtgctggcctacgccaaccccaccctgagcgccgtgtaa

SEQ ID NO: 149 - RNA, 2220 nt, synthetic construct
  misc_feature 1..2220: note = EBOV NP, Zaire 1976, optimized mRNA Sequence
  source 1..2220: mol_type = other RNA
atggacagccgcccccagaagatctggatggcccccagcctgaccgagagcgacatggactaccacaagatcctgaccgccggcctgagcgtgcagcagggcatcgtgcgccagcgcgtgatccccgtgtaccaggtgaacaacctggaggagatctgccagctgatcatccaggccttcgaggccggcgtggacttccaggagagcgccgacagcttcctgctgatgctgtgcctgcaccacgcctaccagggcgactacaagctgttcctggagagcggcgccgtgaagtacctggagggccacggcttccgcttcgaggtgaagaagcgcgacggcgtgaagcgcctggaggagctgctgcccgccgtgagcagcggcaagaacatcaagcgcaccctggccgccatgcccgaggaggagaccaccgaggccaacgccggccagttcctgagcttcgccagcctgttcctgcccaagctggtggtgggcgagaaggcctgcctggagaaggtgcagcgccagatccaggtgcacgccgagcagggcctgatccagtaccccaccgcctggcagagcgtgggccacatgatggtgatcttccgcctgatgcgcaccaacttcctgatcaagttcctgctgatccaccagggcatgcacatggtggccggccacgacgccaacgacgccgtgatcagcaacagcgtggcccaggcccgcttcagcggcctgctgatcgtgaagaccgtgctggaccacatcctgcagaagaccgagcgcggcgtgcgcctgcaccccctggcccgcaccgccaaggtgaagaacgaggtgaacagcttcaaggccgccctgagcagcctggccaagcacggcgagtacgcccccttcgcccgcctgctgaacctgagcggcgtgaacaacctggagcacggcctgttcccccagctgagcgccatcgccctgggcgtggccaccgcccacggcagcaccctggccggcgtgaacgtgggcgagcagtaccagcagctgcgcgaggccgccaccgaggccgagaagcagctgcagcagtacgccgagagccgcgagctggaccacctgggcctggacgaccaggagaagaagatcctgatgaacttccaccagaagaagaacgagatcagcttccagcagaccaacgccatggtgaccctgcgcaaggagcgcctggccaagctgaccgaggccatcaccgccgccagcctgcccaagaccagcggccactacgacgacgacgacgacatccccttccccggccccatcaacgacgacgacaaccccggccaccaggacgacgaccccaccgacagccaggacaccaccatccccgacgtggtggtggaccccgacgacggcagctacggcgagtaccagagctacagcgagaacggcatgaacgcccccgacgacctggtgctgttcgacctggacgaggacgacgaggacaccaagcccgtgcccaaccgcagcaccaagggcggccagcagaagaacagccagaagggccagcacatcgagggccgccagacccagagccgccccatccagaacgtgcccggcccccaccgcaccatccaccacgccagcgcccccctgaccgacaacgaccgccgcaacgagcccagcggcagcaccagcccccgcatgctgacccccatcaacgaggaggccgaccccctggacgacgccgacgacgagaccagcagcctgccccccctggagagcgacgacgaggagcaggaccgcgacggcaccagcaaccgcacccccaccgtggccccccccgcccccgtgtaccgcgaccacagcgagaagaaggagctgccccaggacgagcagcaggaccaggaccacacccaggaggcccgcaaccaggacagcgacaacacccagagcgagcacagcttcgaggagatgtaccgccacatcctgcgcagccagggccccttcgacgccgtgctgtactaccacatgatgaaggacgagcccgtggtgttcagcaccagcgacggcaaggagtacacctaccccgacagcctggaggaggagtaccccccctggctgaccgagaaggaggccatgaacgaggagaaccgcttcgtgaccctggacggccagcagttctactggcccgtgatgaaccacaagaacaagttcatggccatcctgcagcaccaccagtaa

SEQ ID NO: 150 - RNA, 2220 nt, synthetic construct
  misc_feature 1..2220: note = EBOV NP, Sierra Leone 2014, optimized mRNA Sequence
  source 1..2220: mol_type = other RNA
atggacagccgcccccagaaggtgtggatgacccccagcctgaccgagagcgacatggactaccacaagatcctgaccgccggcctgagcgtgcagcagggcatcgtgcgccagcgcgtgatccccgtgtaccaggtgaacaacctggaggagatctgccagctgatcatccaggccttcgaggccggcgtggacttccaggagagcgccgacagcttcctgctgatgctgtgcctgcaccacgcctaccagggcgactacaagctgttcctggagagcggcgccgtgaagtacctggagggccacggcttccgcttcgaggtgaagaagtgcgacggcgtgaagcgcctggaggagctgctgcccgccgtgagcagcggccgcaacatcaagcgcaccctggccgccatgcccgaggaggagaccaccgaggccaacgccggccagttcctgagcttcgccagcctgttcctgcccaagctggtggtgggcgagaaggcctgcctggagaaggtgcagcgccagatccaggtgcacgccgagcagggcctgatccagtaccccaccgcctggcagagcgtgggccacatgatggtgatcttccgcctgatgcgcaccaacttcctgatcaagttcctgctgatccaccagggcatgcacatggtggccggccacgacgccaacgacgccgtgatcagcaacagcgtggcccaggcccgcttcagcggcctgctgatcgtgaagaccgtgctggaccacatcctgcagaagaccgagcgcggcgtgcgcctgcaccccctggcccgcaccgccaaggtgaagaacgaggtgaacagcttcaaggccgccctgagcagcctggccaagcacggcgagtacgcccccttcgcccgcctgctgaacctgagcggcgtgaacaacctggagcacggcctgttcccccagctgagcgccatcgccctgggcgtggccaccgcccacggcagcaccctggccggcgtgaacgtgggcgagcagtaccagcagctgcgcgaggccgccaccgaggccgagaagcagctgcagcagtacgccgagagccgcgagctggaccacctgggcctggacgaccaggagaagaagatcctgatgaacttccaccagaagaagaacgagatcagcttccagcagaccaacgccatggtgaccctgcgcaaggagcgcctggccaagctgaccgaggccatcaccgccgccagcctgcccaagaccagcggccactacgacgacgacgacgacatccccttccccggccccatcaacgacgacgacaaccccggccaccaggacgacgaccccaccgacagccaggacaccaccatccccgacgtggtggtggaccccgacgacggcggctacggcgagtaccagagctacagcgagaacggcatgagcgcccccgacgacctggtgctgttcgacctggacgaggacgacgaggacaccaagcccgtgcccaaccgcagcaccaagggcggccagcagaagaacagccagaagggccagcacaccgagggccgccagacccagagcacccccacccagaacgtgaccggcccccgccgcaccatccaccacgccagcgcccccctgaccgacaacgaccgccgcaacgagcccagcggcagcaccagcccccgcatgctgacccccatcaacgaggaggccgaccccctggacgacgccgacgacgagaccagcagcctgccccccctggagagcgacgacgaggagcaggaccgcgacggcaccagcaaccgcacccccaccgtggccccccccgcccccgtgtaccgcgaccacagcgagaagaaggagctgccccaggacgagcagcaggaccaggaccacatccaggaggcccgcaaccaggacagcgacaacacccagcccgagcacagcttcgaggagatgtaccgccacatcctgcgcagccagggccccttcgacgccgtgctgtactaccacatgatgaaggacgagcccgtggtgttcagcaccagcgacggcaaggagtacacctaccccgacagcctggaggaggagtaccccccctggctgaccgagaaggaggccatgaacgacgagaaccgcttcgtgaccctggacggccagcagttctactggcccgtgatgaaccaccgcaacaagttcatggccatcctgcagcaccaccagtaa

SEQ ID NO: 151 - RNA, 2031 nt, synthetic construct
  misc_feature 1..2031: note = BDBV GP, Uganda 2007,optimized mRNA Sequence
  source 1..2031: mol_type = other RNA
atggtgaccagcggcatcctgcagctgccccgcgagcgcttccgcaagaccagcttcttcgtgtgggtgatcatcctgttccacaaggtgttccccatccccctgggcgtggtgcacaacaacaccctgcaggtgagcgacatcgacaagctggtgtgccgcgacaagctgagcagcaccagccagctgaagagcgtgggcctgaacctggagggcaacggcgtggccaccgacgtgcccaccgccaccaagcgctggggcttccgcgccggcgtgccccccaaggtggtgaactacgaggccggcgagtgggccgagaactgctacaacctggacatcaagaaggccgacggcagcgagtgcctgcccgaggcccccgagggcgtgcgcggcttcccccgctgccgctacgtgcacaaggtgagcggcaccggcccctgccccgagggctacgccttccacaaggagggcgccttcttcctgtacgaccgcctggccagcaccatcatctaccgcagcaccaccttcagcgagggcgtggtggccttcctgatcctgcccgagaccaagaaggacttcttccagagcccccccctgcacgagcccgccaacatgaccaccgaccccagcagctactaccacaccgtgaccctgaactacgtggccgacaacttcggcaccaacatgaccaacttcctgttccaggtggaccacctgacctacgtgcagctggagccccgcttcaccccccagttcctggtgcagctgaacgagaccatctacaccaacggccgccgcagcaacaccaccggcaccctgatctggaaggtgaaccccaccgtggacaccggcgtgggcgagtgggccttctgggagaacaagaagaacttcaccaagaccctgagcagcgaggagctgagcgtgatcttcgtgccccgcgcccaggaccccggcagcaaccagaagaccaaggtgacccccaccagcttcgccaacaaccagaccagcaagaaccacgaggacctggtgcccgaggaccccgccagcgtggtgcaggtgcgcgacctgcagcgcgagaacaccgtgcccacccccccccccgacaccgtgcccaccaccctgatccccgacaccatggaggagcagaccaccagccactacgagccccccaacatcagccgcaaccaccaggagcgcaacaacaccgcccaccccgagaccctggccaacaacccccccgacaacaccacccccagcacccccccccaggacggcgagcgcaccagcagccacaccacccccagcccccgccccgtgcccaccagcaccatccaccccaccacccgcgagacccacatccccaccaccatgaccaccagccacgacaccgacagcaaccgccccaaccccatcgacatcagcgagagcaccgagcccggccccctgaccaacaccacccgcggcgccgccaacctgctgaccggcagccgccgcacccgccgcgagatcaccctgcgcacccaggccaagtgcaaccccaacctgcactactggaccacccaggacgagggcgccgccatcggcctggcctggatcccctacttcggccccgccgccgagggcatctacaccgagggcatcatgcacaaccagaacggcctgatctgcggcctgcgccagctggccaacgagaccacccaggccctgcagctgttcctgcgcgccaccaccgagctgcgcaccttcagcatcctgaaccgcaaggccatcgacttcctgctgcagcgctggggcggcacctgccacatcctgggccccgactgctgcatcgagccccacgactggaccaagaacatcaccgacaagatcgaccagatcatccacgacttcatcgacaagcccctgcccgaccagaccgacaacgacaactggtggaccggctggcgccagtgggtgcccgccggcatcggcatcaccggcgtgatcatcgccgtgatcgccctgctgtgcatctgcaagttcctgctgtaa

SEQ ID NO: 152 - RNA, 2031 nt, synthetic construct
  misc_feature 1..2031: note = SUDV GP, Gulu, Uganda 2007, optimized mRNA Sequence
  source 1..2031: mol_type = other RNA
atgggcggcctgagcctgctgcagctgccccgcgacaagttccgcaagagcagcttcttcgtgtgggtgatcatcctgttccagaaggccttcagcatgcccctgggcgtggtgaccaacagcaccctggaggtgaccgagatcgaccagctggtgtgcaaggaccacctggccagcaccgaccagctgaagagcgtgggcctgaacctggagggcagcggcgtgagcaccgacatccccagcgccaccaagcgctggggcttccgcagcggcgtgccccccaaggtggtgagctacgaggccggcgagtgggccgagaactgctacaacctggagatcaagaagcccgacggcagcgagtgcctgccccccccccccgacggcgtgcgcggcttcccccgctgccgctacgtgcacaaggcccagggcaccggcccctgccccggcgactacgccttccacaaggacggcgccttcttcctgtacgaccgcctggccagcaccgtgatctaccgcggcgtgaacttcgccgagggcgtgatcgccttcctgatcctggccaagcccaaggagaccttcctgcagagcccccccatccgcgaggccgtgaactacaccgagaacaccagcagctactacgccaccagctacctggagtacgagatcgagaacttcggcgcccagcacagcaccaccctgttcaagatcgacaacaacaccttcgtgcgcctggaccgcccccacaccccccagttcctgttccagctgaacgacaccatccacctgcaccagcagctgagcaacaccaccggccgcctgatctggaccctggacgccaacatcaacgccgacatcggcgagtgggccttctgggagaacaagaagaacctgagcgagcagctgcgcggcgaggagctgagcttcgaggccctgagcctgaacgagaccgaggacgacgacgccgccagcagccgcatcaccaagggccgcatcagcgaccgcgccacccgcaagtacagcgacctggtgcccaagaacagccccggcatggtgcccctgcacatccccgagggcgagaccaccctgcccagccagaacagcaccgagggccgccgcgtgggcgtgaacacccaggagaccatcaccgagaccgccgccaccatcatcggcaccaacggcaaccacatgcagatcagcaccatcggcatccgccccagcagcagccagatccccagcagcagccccaccaccgcccccagccccgaggcccagacccccaccacccacaccagcggccccagcgtgatggccaccgaggagcccaccaccccccccggcagcagccccggccccaccaccgaggcccccaccctgaccacccccgagaacatcaccaccgccgtgaagaccgtgctgccccaggagagcaccagcaacggcctgatcaccagcaccgtgaccggcatcctgggcagcctgggcctgcgcaagcgcagccgccgccagaccaacaccaaggccaccggcaagtgcaaccccaacctgcactactggaccgcccaggagcagcacaacgccgccggcatcgcctggatcccctacttcggccccggcgccgagggcatctacaccgagggcctgatgcacaaccagaacgccctggtgtgcggcctgcgccagctggccaacgagaccacccaggccctgcagctgttcctgcgcgccaccaccgagctgcgcacctacaccatcctgaaccgcaaggccatcgacttcctgctgcgccgctggggcggcacctgccgcatcctgggccccgactgctgcatcgagccccacgactggaccaagaacatcaccgacaagatcaaccagatcatccacgacttcatcgacaaccccctgcccaaccaggacaacgacgacaactggtggaccggctggcgccagtggatccccgccggcatcggcatcaccggcatcatcatcgccatcatcgccctgctgtgcgtgtgcaagctgctgtgctaa

SEQ ID NO: 153 - RNA, 2031 nt, synthetic construct
  misc_feature 1..2031: note = TAFV GP, Cote dIvoire 1994, optimized mRNA Sequence
  source 1..2031: mol_type = other RNA
atgggcgccagcggcatcctgcagctgccccgcgagcgcttccgcaagaccagcttcttcgtgtgggtgatcatcctgttccacaaggtgttcagcatccccctgggcgtggtgcacaacaacaccctgcaggtgagcgacatcgacaagttcgtgtgccgcgacaagctgagcagcaccagccagctgaagagcgtgggcctgaacctggagggcaacggcgtggccaccgacgtgcccaccgccaccaagcgctggggcttccgcgccggcgtgccccccaaggtggtgaactgcgaggccggcgagtgggccgagaactgctacaacctggccatcaagaaggtggacggcagcgagtgcctgcccgaggcccccgagggcgtgcgcgacttcccccgctgccgctacgtgcacaaggtgagcggcaccggcccctgccccggcggcctggccttccacaaggagggcgccttcttcctgtacgaccgcctggccagcaccatcatctaccgcggcaccaccttcgccgagggcgtgatcgccttcctgatcctgcccaaggcccgcaaggacttcttccagagcccccccctgcacgagcccgccaacatgaccaccgaccccagcagctactaccacaccaccaccatcaactacgtggtggacaacttcggcaccaacaccaccgagttcctgttccaggtggaccacctgacctacgtgcagctggaggcccgcttcaccccccagttcctggtgctgctgaacgagaccatctacagcgacaaccgccgcagcaacaccaccggcaagctgatctggaagatcaaccccaccgtggacaccagcatgggcgagtgggccttctgggagaacaagaagaacttcaccaagaccctgagcagcgaggagctgagcttcgtgcccgtgcccgagacccagaaccaggtgctggacaccaccgccaccgtgagcccccccatcagcgcccacaaccacgccgccgaggaccacaaggagctggtgagcgaggacagcacccccgtggtgcagatgcagaacatcaagggcaaggacaccatgcccaccaccgtgaccggcgtgcccaccaccacccccagccccttccccatcaacgcccgcaacaccgaccacaccaagagcttcatcggcctggagggcccccaggaggaccacagcaccacccagcccgccaagaccaccagccagcccaccaacagcaccgagagcaccaccctgaaccccaccagcgagcccagcagccgcggcaccggccccagcagccccaccgtgcccaacaccaccgagagccacgccgagctgggcaagaccacccccaccaccctgcccgagcagcacaccgccgccagcgccatcccccgcgccgtgcaccccgacgagctgagcggccccggcttcctgaccaacaccatccgcggcgtgaccaacctgctgaccggcagccgccgcaagcgccgcgacgtgacccccaacacccagcccaagtgcaaccccaacctgcactactggaccgccctggacgagggcgccgccatcggcctggcctggatcccctacttcggccccgccgccgagggcatctacaccgagggcatcatggagaaccagaacggcctgatctgcggcctgcgccagctggccaacgagaccacccaggccctgcagctgttcctgcgcgccaccaccgagctgcgcaccttcagcatcctgaaccgcaaggccatcgacttcctgctgcagcgctggggcggcacctgccacatcctgggccccgactgctgcatcgagccccaggactggaccaagaacatcaccgacaagatcgaccagatcatccacgacttcgtggacaacaacctgcccaaccagaacgacggcagcaactggtggaccggctggaagcagtgggtgcccgccggcatcggcatcaccggcgtgatcatcgccatcatcgccctgctgtgcatctgcaagttcatgctgtaa

SEQ ID NO: 154 - RNA, 981 nt, synthetic construct
  misc_feature 1..981: note = BDBV VP40, Uganda 2007, optimized mRNA Sequence
  source 1..981: mol_type = other RNA
atgcgccgcgccatcctgcccaccgccccccccgagtacatcgaggccgtgtaccccatgcgcaccgtgagcaccagcatcaacagcaccgccagcggccccaacttccccgcccccgacgtgatgatgagcgacacccccagcaacagcctgcgccccatcgccgacgacaacatcgaccaccccagccacacccccaccagcgtgagcagcgccttcatcctggaggccatggtgaacgtgatcagcggccccaaggtgctgatgaagcagatccccatctggctgcccctgggcgtggccgaccagaagacctacagcttcgacagcaccaccgccgccatcatgctggccagctacaccatcacccacttcggcaagaccagcaaccccctggtgcgcatcaaccgcctgggccccggcatccccgaccaccccctgcgcctgctgcgcatcggcaaccaggccttcctgcaggagttcgtgctgccccccgtgcagctgccccagtacttcaccttcgacctgaccgccctgaagctgatcacccagcccctgcccgccgccacctggaccgacgacacccccaccggccccaccggcatcctgcgccccggcatcagcttccaccccaagctgcgccccatcctgctgcccggcaagaccggcaagcgcggcagcagcagcgacctgaccagccccgacaagatccaggccatcatgaacttcctgcaggacctgaagctggtgcccatcgaccccgccaagaacatcatgggcatcgaggtgcccgagctgctggtgcaccgcctgaccggcaagaagatcaccaccaagaacggccagcccatcatccccatcctgctgcccaagtacatcggcatggaccccatcagccagggcgacctgaccatggtgatcacccaggactgcgacacctgccacagccccgccagcctgccccccgtgagcgagaagtaa

SEQ ID NO: 155 - RNA, 981 nt, synthetic construct
  misc_feature 1..981: note = SUDV VP40, Gulu, Uganda 2000, optimized mRNA Sequence
  source 1..981: mol_type = other RNA
atgcgccgcgtgaccgtgcccaccgccccccccgcctacgccgacatcggctaccccatgagcatgctgcccatcaagagcagccgcgccgtgagcggcatccagcagaagcaggaggtgctgcccggcatggacacccccagcaacagcatgcgccccgtggccgacgacaacatcgaccacaccagccacacccccaacggcgtggccagcgccttcatcctggaggccaccgtgaacgtgatcagcggccccaaggtgctgatgaagcagatccccatctggctgcccctgggcatcgccgaccagaagacctacagcttcgacagcaccaccgccgccatcatgctggccagctacaccatcacccacttcggcaaggccaacaaccccctggtgcgcgtgaaccgcctgggccagggcatccccgaccaccccctgcgcctgctgcgcatgggcaaccaggccttcctgcaggagttcgtgctgccccccgtgcagctgccccagtacttcaccttcgacctgaccgccctgaagctggtgacccagcccctgcccgccgccacctggaccgacgagacccccagcaacctgagcggcgccctgcgccccggcctgagcttccaccccaagctgcgccccgtgctgctgcccggcaagaccggcaagaagggccacgtgagcgacctgaccgcccccgacaagatccagaccatcgtgaacctgatgcaggacttcaagatcgtgcccatcgaccccgccaagagcatcatcggcatcgaggtgcccgagctgctggtgcacaagctgaccggcaagaagatgagccagaagaacggccagcccatcatccccgtgctgctgcccaagtacatcggcctggaccccatcagccccggcgacctgaccatggtgatcacccccgactacgacgactgccacagccccgccagctgcagctacctgagcgagaagtaa

SEQ ID NO: 156 - RNA, 981 nt, synthetic construct
  misc_feature 1..981: note = TAFV VP40, Cote dIvoire 1994, optimized mRNA Sequence
  source 1..981: mol_type = other RNA
atgcgccgcatcatcctgcccaccgccccccccgagtacatggaggccgtgtaccccatgcgcaccatgaacagcggcgccgacaacaccgccagcggccccaactacaccaccaccggcgtgatgaccaacgacacccccagcaacagcctgcgccccgtggccgacgacaacatcgaccaccccagccacacccccaacagcgtggccagcgccttcatcctggaggccatggtgaacgtgatcagcggccccaaggtgctgatgaagcagatccccatctggctgcccctgggcgtgagcgaccagaagacctacagcttcgacagcaccaccgccgccatcatgctggccagctacaccatcacccacttcggcaagaccagcaaccccctggtgcgcatcaaccgcctgggccccggcatccccgaccaccccctgcgcctgctgcgcatcggcaaccaggccttcctgcaggagttcgtgctgccccccgtgcagctgccccagtacttcaccttcgacctgaccgccctgaagctgatcacccagcccctgcccgccgccacctggaccgacgagacccccgccgtgagcaccggcaccctgcgccccggcatcagcttccaccccaagctgcgccccatcctgctgcccggccgcgccggcaagaagggcagcaacagcgacctgaccagccccgacaagatccaggccatcatgaacttcctgcaggacctgaagatcgtgcccatcgaccccaccaagaacatcatgggcatcgaggtgcccgagctgctggtgcaccgcctgaccggcaagaagaccaccaccaagaacggccagcccatcatccccatcctgctgcccaagtacatcggcctggaccccctgagccagggcgacctgaccatggtgatcacccaggactgcgacagctgccacagccccgccagcctgccccccgtgaacgagaagtaa

SEQ ID NO: 157 - RNA, 2088 nt, synthetic construct
  misc_feature 1..2088: note = MARV NP, Angola 2005, optimized mRNA Sequence
  source 1..2088: mol_type = other RNA
atggacctgcacagcctgctggagctgggcaccaagcccaccgccccccacgtgcgcaacaagaaggtgatcctgttcgacaccaaccaccaggtgagcatctgcaaccagatcatcgacgccatcaacagcggcatcgacctgggcgacctgctggagggcggcctgctgaccctgtgcgtggagcactactacaacagcgacaaggacaagttcaacaccagccccatcgccaagtacctgcgcgacgccggctacgagttcgacgtgatcaagaacgccgacgccacccgcttcctggacgtgatccccaacgagccccactacagccccctgatcctggccctgaagaccctggagagcaccgagagccagcgcggccgcatcggcctgttcctgagcttctgcagcctgttcctgcccaagctggtggtgggcgaccgcgccagcatcgagaaggccctgcgccaggtgaccgtgcaccaggagcagggcatcgtgacctaccccaaccactggctgaccaccggccacatgaaggtgatcttcggcatcctgcgcagcagcttcatcctgaagttcgtgctgatccaccagggcgtgaacctggtgaccggccacgacgcctacgacagcatcatcagcaacagcgtgggccagacccgcttcagcggcctgctgatcgtgaagaccgtgctggagttcatcctgcagaagaccgacagcggcgtgaccctgcaccccctggtgcgcaccagcaaggtgaagaacgaggtggccagcttcaagcaggccctgagcaacctggcccgccacggcgagtacgcccccttcgcccgcgtgctgaacctgagcggcatcaacaacctggagcacggcctgtacccccagctgagcgccatcgccctgggcgtggccaccgcccacggcagcaccctggccggcgtgaacgtgggcgagcagtaccagcagctgcgcgaggccgcccacgacgccgaggtgaagctgcagcgccgccacgagcaccaggagatccaggccatcgccgaggacgacgaggagcgcaagatcctggagcagttccacctgcagaagaccgagatcacccacagccagaccctggccgtgctgagccagaagcgcgagaagctggcccgcctggccgccgagatcgagaacaacatcgtggaggaccagggcttcaagcagagccagaaccgcgtgagccagagcttcctgaacgaccccacccccgtggaggtgaccgtgcaggcccgccccatcaaccgccccaccgccctgcccccccccgtggacagcaagatcgagcacgagagcaccgaggacagcagcagcagcagcagcttcgtggacctgaacgaccccttcgccctgctgaacgaggacgaggacaccctggacgacagcgtgatgatccccagcaccaccagccgcgagttccagggcatccccgagcccccccgccagagccaggacatcgacaacagccagggcaagcaggaggacgagagcaccaacctgatcaagaagcccttcctgcgctaccaggagctgccccccgtgcaggaggacgacgagagcgagtacaccaccgacagccaggagagcatcgaccagcccggcagcgacaacgagcagggcgtggacctgccccccccccccctgtacgcccaggagaagcgccaggaccccatccagcaccccgccgtgagcagccaggaccccttcggcagcatcggcgacgtgaacggcgacatcctggagcccatccgcagccccagcagccccagcgccccccaggaggacacccgcgcccgcgaggcctacgagctgagccccgacttcaccaactacgaggacaaccagcagaactggccccagcgcgtggtgaccaagaagggccgcaccttcctgtaccccaacgacctgctgcagaccaacccccccgagagcctgatcaccgccctggtggaggagtaccagaaccccgtgagcgccaaggagctgcaggccgactggcccgacatgagcttcgacgagcgccgccacgtggccatgaacctgtaa

SEQ ID NO: 158 - RNA, 2220 nt, synthetic construct
  misc_feature 1..2220: note = BDBV NP, Uganda 2007, optimized mRNA Sequence
  source 1..2220: mol_type = other RNA
atggacccccgccccatccgcacctggatgatgcacaacaccagcgaggtggaggccgactaccacaagatcctgaccgccggcctgagcgtgcagcagggcatcgtgcgccagcgcatcatccccgtgtaccagatcagcaacctggaggaggtgtgccagctgatcatccaggccttcgaggccggcgtggacttccaggacagcgccgacagcttcctgctgatgctgtgcctgcaccacgcctaccagggcgactacaagcagttcctggagagcaacgccgtgaagtacctggagggccacggcttccgcttcgagatgaagaagaaggagggcgtgaagcgcctggaggagctgctgcccgccgccagcagcggcaagaacatcaagcgcaccctggccgccatgcccgaggaggagaccaccgaggccaacgccggccagttcctgagcttcgccagcctgttcctgcccaagctggtggtgggcgagaaggcctgcctggagaaggtgcagcgccagatccaggtgcacgccgagcagggcctgatccagtaccccaccagctggcagagcgtgggccacatgatggtgatcttccgcctgatgcgcaccaacttcctgatcaagttcctgctgatccaccagggcatgcacatggtggccggccacgacgccaacgacgccgtgatcgccaacagcgtggcccaggcccgcttcagcggcctgctgatcgtgaagaccgtgctggaccacatcctgcagaagaccgagcacggcgtgcgcctgcaccccctggcccgcaccgccaaggtgaagaacgaggtgagcagcttcaaggccgccctggccagcctggcccagcacggcgagtacgcccccttcgcccgcctgctgaacctgagcggcgtgaacaacctggagcacggcctgttcccccagctgagcgccatcgccctgggcgtggccaccgcccacggcagcaccctggccggcgtgaacgtgggcgagcagtaccagcagctgcgcgaggccgccaccgaggccgagaagcagctgcagaagtacgccgagagccgcgagctggaccacctgggcctggacgaccaggagaagaagatcctgaaggacttccaccagaagaagaacgagatcagcttccagcagaccaccgccatggtgaccctgcgcaaggagcgcctggccaagctgaccgaggccatcaccagcaccagcatcctgaagaccggccgccgctacgacgacgacaacgacatccccttccccggccccatcaacgacaacgagaacagcggccagaacgacgacgaccccaccgacagccaggacaccaccatccccgacgtgatcatcgaccccaacgacggcggctacaacaactacagcgactacgccaacgacgccgccagcgcccccgacgacctggtgctgttcgacctggaggacgaggacgacgccgacaaccccgcccagaacacccccgagaagaacgaccgccccgccaccaccaagctgcgcaacggccaggaccaggacggcaaccagggcgagaccgccagcccccgcgtggcccccaaccagtaccgcgacaagcccatgccccaggtgcaggaccgcagcgagaaccacgaccagaccctgcagacccagagccgcgtgctgacccccatcagcgaggaggccgaccccagcgaccacaacgacggcgacaacgagagcatcccccccctggagagcgacgacgagggcagcaccgacaccaccgccgccgagaccaagcccgccaccgccccccccgcccccgtgtaccgcagcatcagcgtggacgacagcgtgcccagcgagaacatccccgcccagagcaaccagaccaacaacgaggacaacgtgcgcaacaacgcccagagcgagcagagcatcgccgagatgtaccagcacatcctgaagacccagggccccttcgacgccatcctgtactaccacatgatgaaggaggagcccatcatcttcagcaccagcgacggcaaggagtacacctaccccgacagcctggaggacgagtaccccccctggctgagcgagaaggaggccatgaacgaggacaaccgcttcatcaccatggacggccagcagttctactggcccgtgatgaaccaccgcaacaagttcatggccatcctgcagcaccaccgctaa

SEQ ID NO: 159 - RNA, 2217 nt, synthetic construct
  misc_feature 1..2217: note = SUDV NP, Gulu, Uganda 2000,optimized mRNA Sequence
  source 1..2217: mol_type = other RNA
atggacaagcgcgtgcgcggcagctgggccctgggcggccagagcgaggtggacctggactaccacaagatcctgaccgccggcctgagcgtgcagcagggcatcgtgcgccagcgcgtgatccccgtgtacgtggtgagcgacctggagggcatctgccagcacatcatccaggccttcgaggccggcgtggacttccaggacaacgccgacagcttcctgctgctgctgtgcctgcaccacgcctaccagggcgaccaccgcctgttcctgaagagcgacgccgtgcagtacctggagggccacggcttccgcttcgaggtgcgcgagaaggagaacgtgcaccgcctggacgagctgctgcccaacgtgaccggcggcaagaacctgcgccgcaccctggccgccatgcccgaggaggagaccaccgaggccaacgccggccagttcctgagcttcgccagcctgttcctgcccaagctggtggtgggcgagaaggcctgcctggagaaggtgcagcgccagatccaggtgcacgccgagcagggcctgatccagtaccccaccagctggcagagcgtgggccacatgatggtgatcttccgcctgatgcgcaccaacttcctgatcaagttcctgctgatccaccagggcatgcacatggtggccggccacgacgccaacgacaccgtgatcagcaacagcgtggcccaggcccgcttcagcggcctgctgatcgtgaagaccgtgctggaccacatcctgcagaagaccgacctgggcgtgcgcctgcaccccctggcccgcaccgccaaggtgaagaacgaggtgagcagcttcaaggccgccctgggcagcctggccaagcacggcgagtacgcccccttcgcccgcctgctgaacctgagcggcgtgaacaacctggagcacggcctgtacccccagctgagcgccatcgccctgggcgtggccaccgcccacggcagcaccctggccggcgtgaacgtgggcgagcagtaccagcagctgcgcgaggccgccaccgaggccgagaagcagctgcagcagtacgccgagacccgcgagctggacaacctgggcctggacgagcaggagaagaagatcctgatgagcttccaccagaagaagaacgagatcagcttccagcagaccaacgccatggtgaccctgcgcaaggagcgcctggccaagctgaccgaggccatcaccaccgccagcaagatcaaggtgggcgaccgctaccccgacgacaacgacatccccttccccggccccatctacgacgagacccaccccaaccccagcgacgacaaccccgacgacagccgcgacaccaccatccccggcggcgtggtggacccctacgacgacgagagcaacaactaccccgactacgaggacagcgccgagggcaccaccggcgacctggacctgttcaacctggacgacgacgacgacgacagccagcccggcccccccgaccgcggccagagcaaggagcgcgccgcccgcacccacggcctgcaggaccccaccctggacggcgccaagaaggtgcccgagctgacccccggcagccaccagcccggcaacctgcacatcaccaagcccggcagcaacaccaaccagccccagggcaacatgagcagcaccctgcagagcatgacccccatccaggaggagagcgagcccgacgaccagaaggacgacgacgacgagagcctgaccagcctggacagcgagggcgacgaggacgtggagagcgtgagcggcgagaacaaccccaccgtggccccccccgcccccgtgtacaaggacaccggcgtggacaccaaccagcagaacggccccagcaacgccgtggacggccagggcagcgagagcgaggccctgcccatcaaccccgagaagggcagcgccctggaggagacctactaccacctgctgaagacccagggccccttcgaggccatcaactactaccacctgatgagcgacgagcccatcgccttcagcaccgagagcggcaaggagtacatcttccccgacagcctggaggaggcctaccccccctggctgagcgagaaggaggccctggagaaggagaaccgctacctggtgatcgacggccagcagttcctgtggcccgtgatgagcctgcaggacaagttcctggccgtgctgcagcacgactaa

SEQ ID NO: 160 - RNA, 2220 nt, synthetic construct
  misc_feature 1..2220: note = TAFV NP, Cote dIvoire 1994,optimized mRNA Sequence
  source 1..2220: mol_type = other RNA
atggagagccgcgcccacaaggcctggatgacccacaccgccagcggcttcgagaccgactaccacaagatcctgaccgccggcctgagcgtgcagcagggcatcgtgcgccagcgcgtgatccaggtgcaccaggtgaccaacctggaggagatctgccagctgatcatccaggccttcgaggccggcgtggacttccaggagagcgccgacagcttcctgctgatgctgtgcctgcaccacgcctaccagggcgactacaagcagttcctggagagcaacgccgtgaagtacctggagggccacggcttccgcttcgaggtgcgcaagaaggagggcgtgaagcgcctggaggagctgctgcccgccgccagcagcggcaagagcatccgccgcaccctggccgccatgcccgaggaggagaccaccgaggccaacgccggccagttcctgagcttcgccagcctgttcctgcccaagctggtggtgggcgagaaggcctgcctggagaaggtgcagcgccagatccaggtgcacagcgagcagggcctgatccagtaccccaccgcctggcagagcgtgggccacatgatggtgatcttccgcctgatgcgcaccaacttcctgatcaagttcctgctgatccaccagggcatgcacatggtggccggccacgacgccaacgacgccgtgatcgccaacagcgtggcccaggcccgcttcagcggcctgctgatcgtgaagaccgtgctggaccacatcctgcagaagaccgagcacggcgtgcgcctgcaccccctggcccgcaccgccaaggtgaagaacgaggtgaacagcttcaaggccgccctgagcagcctggcccagcacggcgagtacgcccccttcgcccgcctgctgaacctgagcggcgtgaacaacctggagcacggcctgttcccccagctgagcgccatcgccctgggcgtggccaccgcccacggcagcaccctggccggcgtgaacgtgggcgagcagtaccagcagctgcgcgaggccgccaccgaggccgagaagcagctgcagaagtacgccgagagccgcgagctggaccacctgggcctggacgaccaggagaagaagatcctgaaggacttccaccagaagaagaacgagatcagcttccagcagaccaccgccatggtgaccctgcgcaaggagcgcctggccaagctgaccgaggccatcaccagcaccagcctgctgaagaccggcaagcagtacgacgacgacaacgacatccccttccccggccccatcaacgacaacgagaacagcgagcagcaggacgacgaccccaccgacagccaggacaccaccatccccgacatcatcgtggaccccgacgacggccgctacaacaactacggcgactaccccagcgagaccgccaacgcccccgaggacctggtgctgttcgacctggaggacggcgacgaggacgaccaccgccccagcagcagcagcgagaacaacaacaagcacagcctgaccggcaccgacagcaacaagaccagcaactggaaccgcaaccccaccaacatgcccaagaaggacagcacccagaacaacgacaaccccgcccagcgcgcccaggagtacgcccgcgacaacatccaggacacccccaccccccaccgcgccctgacccccatcagcgaggagaccggcagcaacggccacaacgaggacgacatcgacagcatcccccccctggagagcgacgaggagaacaacaccgagaccaccatcaccaccaccaagaacaccaccgccccccccgcccccgtgtaccgcagcaacagcgagaaggagcccctgccccaggagaagagccagaagcagcccaaccaggtgagcggcagcgagaacaccgacaacaagccccacagcgagcagagcgtggaggagatgtaccgccacatcctgcagacccagggccccttcgacgccatcctgtactactacatgatgaccgaggagcccatcgtgttcagcaccagcgacggcaaggagtacgtgtaccccgacagcctggagggcgagcaccccccctggctgagcgagaaggaggccctgaacgaggacaaccgcttcatcaccatggacgaccagcagttctactggcccgtgatgaaccaccgcaacaagttcatggccatcctgcagcaccacaagtaa

SEQ ID NO: 161 - RNA, 2031 nt, synthetic construct
  misc_feature 1..2031: note = EBOV GP, Mayinga, Zaire 1976, optimized mRNA Sequence
  source 1..2031: mol_type = other RNA
atgggggtgacgggaattctgcagctacctagagatcggtttaagcggactagctttttcctctgggtcattatcctgttccaaaggaccttctctattccattaggggtcattcataactctacgctgcaggtgtcggatgttgataaactggtgtgccgtgacaaactgagttccacaaaccaactgcgctctgtggggcttaacttggaaggtaacggtgtggcgacagacgttccgtccgcaacaaagaggtgggggttccgatccggagtccccccaaaggtagtgaattacgaggccggggagtgggcagagaattgctataatctcgagattaaaaaacctgacggctctgaatgcctgccagcggcccctgatggtatccgcggattccctcggtgtaggtacgtgcacaaagttagcggaaccggcccgtgcgctggcgactttgcattccataaggaaggtgcttttttcctctacgaccggttggcatccacggtaatctatcggggaacaactttcgccgagggtgtggtagccttcttaatcctccctcaggccaaaaaagacttcttctcctctcacccattgagggagcctgtgaatgcgaccgaagacccttcctcagggtactatagcaccacaatacgttatcaagccaccggttttgggactaacgagaccgagtatctctttgaagtcgacaacctgacttacgtacagctggagagtaggttcacccctcagttcctccttcagctaaatgagaccatctatacgagtggaaaacgttctaacaccactggtaaactaatatggaaagtgaacccagaaattgacacaaccattggagaatgggccttttgggagaccaagaagaacctgacccggaaaatcagaagtgaagagctgtcgtttacagtagtcagtaatggtgctaaaaacatttcaggtcagtccccagcgcgtacttcaagtgacccgggtaccaacacaacaactgaggatcataaaatcatggcaagcgagaattcctctgccatggttcaggttcacagccaaggtcgagaggcggccgtgtcacacctgactactttggccactatttccacctctcctcaatccctcactaccaaacctggccctgataattccacccacaatacccctgtttacaaactggacattagcgaggccactcaggtggaacagcaccaccgcaggactgataatgactcaaccgcgagtgacacaccgagcgcaactacagcagcgggaccgccgaaggctgaaaacactaacaccagcaaaagtacggatttcttggaccccgccacaactacttcacctcaaaatcatagtgaaactgctgggaataataatactcaccatcaggatactggggaagaatctgcttccagcggcaagctgggactgattacaaataccatcgcaggcgtggcgggcctgattaccggcggtaggaggaccaggagagaggctatagtgaatgcacaacccaaatgcaatcccaatctccactattggactacgcaagacgagggcgctgctatcgggctggcgtggatcccctatttcggccccgccgctgagggaatatatattgaaggcctgatgcacaaccaagacggcctgatttgcggtctccggcaactcgccaacgagaccacccaagctctgcagctgtttcttagggccaccaccgaactccgtactttcagcatcctcaaccgcaaggccatcgacttcttactccagcggtgggggggaacctgtcatatactcggccctgactgctgtattgagccccacgactggactaagaatatcacagacaaaatcgaccagattattcacgacttcgtcgataaaactctacccgaccagggcgacaacgataattggtggaccggctggagacaatggattccagccggtatcggcgtcaccggcgtgatcatcgctgtgattgccctcttctgcatttgtaagttcgtcttttag

SEQ ID NO: 162 - RNA, 2031 nt, synthetic construct
  misc_feature 1..2031: note = EBOV GP, Sierra Leone 2014, optimized mRNA Sequence
  source 1..2031: mol_type = other RNA
atgggagtcacaggtatcctccagctcccacgggataggttcaagcgcacctcatttttcctgtgggttattattttattccagcgcaccttttccattcccttaggtgtgatccacaattctactcttcaagtgagcgatgttgataagctggtctgtcgcgataaattgagctccaccaaccagctgagaagtgtgggactaaatcttgagggcaatggcgtagctactgacgttccctccgttacaaagcgctgggggtttcgttccggagtgccccccaaagtcgttaactacgaagctggagagtgggcagaaaactgctacaacctcgaaataaaaaagccggatggatccgaatgcctgccggccgctcctgacggcattcgaggatttcctcgatgcaggtatgtacacaaagtctccggtactggcccatgtgcaggcgattttgcttttcacaaagagggtgcattctttctttacgatcgcctggcttctactgtgatatataggggcaccacgttcgctgaaggcgtagtcgccttcctaattctgcctcaggcaaagaaggattttttctcctcccatcccctcagagaaccagtgaacgctaccgaagatcccagtagcgggtattactcaaccaccatcaggtatcaggccaccgggtttggaactaatgaaacagagtacctcttcgaagtggataatctgacatatgtgcagcttgaatctagattcaccccccaattcctgctccagctgaatgagactatttacgctagcgggaagaggtcgaacaccaccggcaagctcatatggaaggttaaccccgagattgacaccacgataggcgagtgggctttttgggagactaaaaagaacttgactcggaagataagatctgaagagctgagcttcaccgccgtctcgaacgggcccaagaacatctctggtcagtcgccagccaggacctctagcgaccccgagaccaataccacaaatgaggaccacaagattatggcctccgagaattccagcgccatggttcaggtgcactcccagggccgaaaggcggcagtttcacatctgactactttggcgactatttctacttcgccccaaccccccactacgaagactgggccagataattcaacccataatactcctgtctacaaactggacatctctgaggcaactcaggtgggacagcatcataggcgcgccgacaatgatagtaccgcttccgacaccccccctgccacaacagctgccggaccactgaaagccgagaatactaatacatccaagagtgccgactcgctggaccttgccactaccacttcaccacaaaattacagcgaaactgccggaaacaacaacacccatcatcaggataccggagaggaatcagccagctcaggaaagctaggtctgataacgaacactatcgcaggtgtggccgggcttattacagggggaaggagaacccggcgagaagtaatcgtgaatgcccaaccgaagtgtaacccaaatctccactactggactactcaggacgaaggtgccgctatcggactggcttggattccctactttgggcctgccgctgagggcatttacactgaaggtttaatgcacaatcaggatgggcttatttgcggcctgcgccagttagccaacgagacgacacaggctttacagctgtttctgcgggccactaccgagttaaggaccttctccatcttgaacaggaaggccattgatttcctactgcagcggtggggcggaacctgccacatcctggggcctgactgctgcatagaacctcacgactggactaaaaacatcaccgacaagattgatcagattatacacgatttcgtcgataagactttgccagatcaaggcgacaatgacaattggtggacgggttggcggcagtggatcccagctgggattggtgtgactggcgtcattatagctgtcattgctctgttttgtatctgtaaattcgtattctag

SEQ ID NO: 163 - RNA, 2046 nt, synthetic construct
  misc_feature 1..2046: note = MARV GP, Angola 2005, optimized mRNA Sequence
  source 1..2046: mol_type = other RNA
atgaagaccacttgcttgctgatcagcctgattctgattcagggggtgaagacgttaccaatcctggagatcgccagtaacattcagccgcagaatgtggattcagtgtgctccggtacccttcagaaaaccgaggacgtccacctgatgggctttactctgagcggacagaaggtggccgactcgccacttgaggctagcaagcggtgggcattccgcgcaggtgttccacccaagaacgtagaatacacagaaggggaagaggccaagacttgttataacatttccgttactgacccatctggaaaatcccttctcctcgacccacctaccaatatcagagattaccccaaatgtaaaactatccatcacatccaggggcagaatccccatgcccaaggaatcgccttacatctctggggcgcgtttttcctctatgacaggatcgcttctacaaccatgtatcgtggcaaggtattcaccgagggcaacattgctgcgatgattgtcaataagaccgtccacaagatgattttctctcgacaggggcaggggtataggcatatgaatcttacctctacaaataaatactggacctcttcaaatgggacacagaccaacgatacaggctgttttggtactctgcaagaatacaattccactaagaaccaaacctgtgccccttccaagaaaccattgcctctcccaacagctcaccccgaagtcaagctcacctcgaccagcaccgacgctaccaagttgaacacaaccgatccgaacagcgatgatgaggacctgacaactagcggaagcggcagcggcgagcaggaaccctataccacctcagacgcagccacaaagcaggggctgtcaagtacgatgccacccacacccagtccccagcccagcacaccgcagcaggggggtaacaataccaatcattcccagggggtggtaacagagcctggcaagacaaatacaaccgcccagccttccatgccgcctcacaacaccacaacaatttctactaataataccagtaaacataatttgagcaccccctccgttccaattcagaatgccacaaactacaacacacagtcaaccgcccctgagaatgagcagacaagcgccccttccaagaccactctgctgcctacggagaatcccacaacggctaaatcgacgaattctaccaaatcccccacaacaactgtcccaaatacaacgaataaatattccacttccccatctccaactccaaacagcacagcacagcacctggtgtattttaggagaaagagaaacattctctggagggaaggtgatatgtttcctttcttggatggattgataaatgctccaatagacttcgaccctgtgccgaatactaagacgatatttgatgagagcagtagcagcggcgcctctgctgaagaagaccagcatgcgtcgccgaacatttcactgaccttaagttactttcccaaggtcaacgaaaataccgcacactcaggcgaaaacgaaaacgactgtgatgctgaactgcgtatttggtcagtgcaggaggatgatctggcagcaggtctcagctggataccattctttggtcctgggatcgaggggctgtacacggcaggcttaatcaaaaaccagaacaatctggtgtgcaggctgcgtcgcctggctaatcagactgccaagtccctcgaattgcttctgagggtcaccaccgaagaaagaacattctccctgatcaataggcacgcaatagacttcttgttggctcgatggggaggaacgtgcaaggtcctcggacccgactgttgcattgggatcgaagatctcagccgaaatatctctgaacagatcgaccagatcaagaaagatgagcaaaaagagggcacaggctggggactcggagggaaatggtggacttcggactggggtgtcctgaccaacctcggtattcttctgctcctgagtatcgcagttttaatcgccctgtcttgcatctgcaggatcttcactaaatacatcggctaa

SEQ ID NO: 164 - RNA, 981 nt, synthetic construct
  misc_feature 1..981: note = EBOV VP40, Mayinga, Zaire 1976, optimized mRNA Sequence
  source 1..981: mol_type = other RNA
atgagacgggtcatcctacctacagcccccccggaatatatggaggctatttatccagttcggagcaactccactatcgccaggggaggcaatagtaacacagggtttctcactcctgagagcgttaatggggacacaccttcgaatccactgcggccaatcgcagacgatactatcgaccacgcgtcccatacacctggatctgtaagtagcgccttcattttggaagctatggtgaatgttatctcagggcctaaagtgttaatgaaacagattcctatttggctgcctttaggtgtggcagatcaaaagacctatagctttgacagtaccacagctgcaatcatgcttgcttcctacaccataacacacttcggcaaggctaccaaccctttagtgcgcgtgaatcggctcggccctggtatcccagatcaccctctcaggctcctgagaataggaaaccaggcattcctacaggaatttgtgttgcctcctgtgcagctgcctcagtactttacatttgatcttaccgcattgaagctaatcacgcagcccctgccggctgctacgtggacagacgatacgcctactggcagtaatggcgccctcaggccaggaatttccttccatcctaaattgcgccccatcctgctcccaaataaaagcggaaaaaaaggaaattcagcggacctgacttctcctgaaaagatccaagctattatgacttcactccaggacttcaaaattgtgcctatcgacccaacaaaaaacataatgggcatagaagtgcctgaaacactggtgcacaagcttactggtaagaaggtcaccagtaagaacggccagcccattatccctgttttattacccaagtacatcggtctggacccagtggcgcccggagatctgacaatggtgattacccaggactgtgacacttgccacagtccagcaagtctgcccgctgtgattgaaaagtaa

SEQ ID NO: 165 - RNA, 981 nt, synthetic construct
  misc_feature 1..981: note = EBOV VP40, Sierra Leone 2014,optimized mRNA Sequence
  source 1..981: mol_type = other RNA
atgcggcgtgtgattctgccgacggccccacccgaatacatggaggctatctacccagctagatcgaacagcacaatcgcaagaggcggcaattcaaacaccggttttcttactcctgagagtgtaaacggcgacaccccttcaaacccgctcagacccattgccgatgacaccatagatcacgcttcacacaccccaggaagtgtgtcgtcagcctttattctggaggccatggtcaacgtgatatctggcccgaaggtgctgatgaaacagatccccatttggttgccactaggggtagctgatcagaaaacatactcctttgatagtacaactgccgcaatcatgctcgcttcttataccatcacacatttcgggaaagcaaccaatcccctagtgcgcgtgaatagattgggccctgggatacctgatcacccactgcgcctgctgcgaatcggaaatcaggccttcctgcaggaatttgtgctgccaccggtgcagctaccgcaatacttcactttcgatctgacagctttaaagctgatcactcagcccctgcccgccgccacatggacagacgacacccccacaggttctaacggcgcactgcggcccgggatctccttccacccaaagctgaggcccatcctcctgcctaataagtctggaaaaaaaggtaacagcgctgacctgacctcccctgaaaagattcaggctataatgacaagtctgcaggattttaaaatagtacctatcgacccaaccaagaacattatgggtatcgaagtgccggaaacactggtccataagttgactggaaagaaagtcacatcaaagaacgggcagcctatcatcccagttctcctgcccaagtacatcggtctcgacccagtcgcgccaggtgatctgacaatggtgattacacaggactgtgatacctgccacagccctgcttcattacccgctgtagttgaaaagtaa

SEQ ID NO: 166 - RNA, 912 nt, synthetic construct
  misc_feature 1..912: note = MARV VP40, Angola 2005,optimized mRNA Sequence
  source 1..912: mol_type = other RNA
atggcttccagtagcaattataacacttacatgcaatacctgaacccgccgccttacgccgaccatggggcaaaccagctcatccccgcagaccagctgtccaatcagcagggaatcacccccaactacgtcggcgaccttaatctggacgaccagtttaagggaaacgtatgtcatgctttcactctggaggccattattgacatcagcgcctataatgaacggaccgtgaagggagtaccagcttggctgcccctggggattatgtctaattttgaatacccgctggcacataccgtagcggcactacttacaggtagctacacgatcacgcagttcactcacaatgggcagaaattcgtacgagttaaccgtctgggcacaggtatacccgcgcacccgcttagaatgctgcgcgagggcaatcaggcttttattcagaatatggtaatcccgaggaacttcagcacaaatcagttcacttataacttaactaacctggttctgagcgtgcaaaagctccctgatgacgcctggaggcctagcaaggacaagctgataggcaacaccatgcatccagccgtcagtgtccacccaaatctgccacccatcgttctgccaacagtcaaaaagcaggcttaccgccagcataaaaatcctaacaatggccccctgctggctatttcaggaattcttcatcagctgcgggtagagaaagtgccggagaaaacttctcttttccgaatctctctacctgccgacatgttttcagtcaaagaaggtatgatgaagaaacgaggggagaatagccccgtcgtgtattttcaggcacctgaaaattttccattgaacggattcaacaataggcaggtcgttctcgcctacgcaaaccccaccttgtccgcagtctaa

SEQ ID NO: 167 - RNA, 2220 nt, synthetic construct
  misc_feature 1..2220: note = EBOV NP, Zaire 1976, optimized mRNA Sequence
  source 1..2220: mol_type = other RNA
atggattcccggcctcagaaaatttggatggcaccctctctgactgagtcggacatggattaccacaaaatcttaacagccggtctgtctgtccagcagggaatcgtgaggcagagagtgattcccgtctatcaggtcaacaacctcgaagaaatttgtcagctgattattcaggccttcgaggcaggcgttgatttccaggaaagtgccgatagtttccttctgatgctgtgccttcatcacgcctatcagggcgattacaagctgtttctcgaaagcggagccgttaagtatttggaaggccatgggtttcggttcgaggtcaaaaagcgcgatggagtaaagagactggaagaactccttccagccgtttccagcggtaaaaacattaaacggaccctcgcagctatgcccgaagaggagaccacagaggctaatgccggacagttcttaagcttcgcatctctgttcttgcctaagctcgtcgttggggagaaggcttgccttgaaaaagtgcagcgccagatacaggtccacgctgaacaggggctgattcagtacccgactgcctggcagtctgtaggccacatgatggtgatcttccgtcttatgcgcacaaacttcttgattaagtttctgctgattcaccagggaatgcacatggtcgctggccacgacgctaatgatgcagtgataagcaacagtgttgcacaagccaggttttccggcctgctgattgtgaagacggtacttgatcacatcctccagaaaacagagcgaggcgtacgacttcatccgcttgctcgcacagctaaagtcaaaaatgaagtgaacagcttcaaagctgcattatcatccctggcaaagcacggtgaatacgccccctttgccagactcctcaacctcagtggggtgaacaaccttgagcacgggctgttcccccagctcagcgccattgcactgggtgtggcgaccgcccatgggtctacactcgcaggtgttaacgtgggggagcagtatcagcagctgcgcgaagccgccactgaagccgagaaacagctgcaacaatatgccgaatccagggagttagaccacctaggactcgatgaccaggagaaaaagatcctgatgaatttccaccagaagaagaatgaaatctcatttcaacagacaaatgctatggtgacacttagaaaagagcgcctggcgaagctgaccgaagctatcacggccgcttcacttcccaagacgagcggtcattacgacgatgacgatgatattccattccccggtccaatcaatgatgatgacaaccccggccaccaggatgatgatccgacagatagccaggataccaccattcctgacgtcgtagtagacccggacgacggaagctatggggaataccaatcgtattctgagaacggcatgaatgccccagacgaccttgtcttatttgatctcgacgaggatgacgaagataccaaacccgttcccaatcgctctaccaaaggtggtcagcagaaaaatagccaaaagggtcagcacatcgagggccgacagacccaaagcagacccattcagaatgtgcctggaccacacaggacaatccaccacgcttcagcacctcttacagacaatgatcggcgaaacgagccaagtgggtccacgagccctcggatgcttactcccataaacgaagaggccgatccactggacgacgctgacgacgaaacgtcttcactccctcctttggaatcggatgacgaggagcaggatagagatgggacatccaaccgtacccctacggtagctcctcctgcacccgtttacagggaccactctgagaagaaggagctgccccaggacgagcaacaggaccaggatcatacacaggaagcacgcaatcaggattctgataatacgcagtctgagcacagcttcgaggagatgtatcgacatatcctccgttctcaaggtcctttcgacgccgtgctgtattaccatatgatgaaggatgagcccgttgtatttagcacttctgacggtaaggaatacacgtatccagattccttggaagaggagtacccaccatggctgacggagaaagaagccatgaatgaggaaaatagatttgtgacgctggatggacagcagttctactggccagttatgaatcacaaaaataaatttatggctatccttcagcatcaccaatga

SEQ ID NO: 168 - RNA, 2220 nt, synthetic construct
  misc_feature 1..2220: note = EBOV NP, Sierra Leone 2014, optimized mRNA Sequence
  source 1..2220: mol_type = other RNA
atggatagcaggcctcaaaaggtctggatgacaccctcacttaccgaaagcgatatggattaccataagattctgacagcgggcctgtctgtgcagcagggaatcgtgagacagagggtgatcccagtttaccaagtaaacaacctggaggagatctgtcagctgataattcaggccttcgaggctggagtcgactttcaggaatcagcggacagcttcttgctgatgctatgcctgcatcacgcctaccaaggggactacaagctgtttctggagtcaggagctgttaagtaccttgaagggcatggcttccgatttgaagtgaaaaagtgtgacggcgttaagcgactggaggaactgcttcccgctgtgagctccggaagaaatataaaacgcactctagcagccatgccggaagaggagaccacagaagccaacgccggccagtttttaagctttgcctcattgttcttgccgaagttggttgtgggcgagaaggcctgcctggagaaggtacaacgacagatccaggtacatgccgagcaaggcctgatccagtacccgactgcgtggcaatctgtgggccacatgatggtcattttcaggctcatgcgcaccaattttctgattaagtttctcttaattcaccaggggatgcacatggtagctgggcatgatgcaaacgatgctgtgatcagcaattccgttgcccaggctcgcttcagcggcttgctgatcgtgaagaccgtgctggatcatatactccagaaaactgaacgcggcgtgaggcttcaccctctcgccagaaccgccaaagtaaaaaatgaggtaaattcatttaaggctgccctgtctagcctcgccaaacatggagaatatgcccccttcgccaggcttttgaacctgtcaggtgtgaacaacctcgaacatggcttgtttccacaactcagtgccatcgcactgggcgttgcaacagcccacggctcgacgctcgctggggtgaatgtgggggagcagtatcagcagcttcgagaagccgcaacagaggccgagaagcagctacagcagtatgctgagagccgtgaactggaccatcttggcttggatgatcaggaaaagaagatcctgatgaatttccaccagaaaaaaaatgagattagtttccagcagacaaatgcaatggttacgctgcggaaagaaaggctcgctaagttaacagaagcgatcacggccgcttctctccccaagacatctgggcattacgacgacgatgatgacatcccttttcccggccctattaacgacgatgataatcctgggcaccaggatgatgatccaacggacagccaggataccactatcccggacgtagtcgtggacccagatgatggggggtacggtgaataccaatcttactcagaaaacggaatgagtgccccagatgaccttgtgttgtttgatctggacgaggacgatgaggacaccaagcctgtcccgaaccgatctaccaagggcggccagcagaagaactctcaaaaaggccagcacaccgagggtcgtcagacacagagtactccaacccaaaacgtaactggccccagacgcacaatacaccatgcatcagctcctctgaccgacaatgatagacggaatgaaccgtcaggttctacttcgccccgcatgctcacccccattaacgaggaagctgaccctctcgacgatgctgatgatgagaccagcagcctgcctccattggaaagcgatgacgaagagcaggacagggatgggaccagcaatagaacccctactgtcgctcccccagctccagtttatagagaccattcggagaaaaaggaacttccacaggatgagcagcaggaccaggatcacatacaggaggctaggaaccaggattctgataacactcaacccgaacactccttcgaagagatgtaccggcacatcctccgatctcagggacccttcgatgctgtgttatattatcacatgatgaaagatgagcccgtcgtcttctctacctccgacggcaaggagtacacttatccggatagtctagaggaggagtacccgccatggctcacagagaaagaagctatgaatgatgaaaatagattcgtcactctcgatgggcagcagttctactggcccgtcatgaatcatagaaataagtttatggccatcttacagcatcatcagtga

SEQ ID NO: 169 - RNA, 2031 nt, synthetic construct
  misc_feature 1..2031: note = BDBV GP, Uganda 2007,optimized mRNA Sequence
  source 1..2031: mol_type = other RNA
atggtcacctctggcatcttgcagctcccccgagaacggttccggaaaacttcattttttgtctgggtcatcatcctttttcataaggttttccccatcccgctcggggtggtgcataataatacattgcaggtatcagacatcgacaagctggtctgtcgtgataagctttcaagcacatcacagcttaagtcagtggggcttaatttagaaggcaacggcgtggctacggacgtgccaaccgccactaagcggtggggcttcagggccggagtcccaccaaaagtcgtaaactacgaggcgggggaatgggctgagaactgttacaacctggatataaaaaaggccgacggctccgaatgcctgcccgaagcaccagagggcgtgcgcggtttcccgcgctgtcgttatgttcataaggtatctgggaccggcccatgtccagaaggctatgcatttcacaaagaaggcgcatttttcctgtatgacaggctggcaagcacaatcatataccgctctaccacattttccgagggtgtagttgcttttcttatcctccccgaaactaagaaggatttttttcagagtcctccactccacgagcctgctaacatgacaactgacccctcttcctactatcacacagtgactttgaactatgtggcggataactttggcacaaacatgactaatttcttgtttcaggtggatcacctcacatacgtgcaactggaaccgagatttactccacagttcctagtgcagctgaatgaaaccatttatacaaacggacgccgctctaacacaaccggcaccctcatctggaaggtcaaccctaccgtggacaccggtgtaggtgaatgggccttctgggagaataaaaaaaacttcaccaaaaccttatccagtgaagagctgtctgttatctttgtgcccagggcgcaggatccgggaagcaatcaaaagactaaggtgacccccaccagcttcgcaaacaaccagacttcaaaaaatcacgaagatcttgtccctgaagaccccgcttcagtagtacaggttcgggatctgcagcgggaaaacacggtccccacaccaccacccgacactgttcctaccaccctcatccccgataccatggaggagcaaactacgtcgcactacgagccgcctaatatatcaagaaaccatcaggaacgtaacaatactgctcatcccgagacactggccaataatcctcccgacaatactacgccgtcaacgcctccacaggacggcgagagaactagtagtcacacaacaccatctcccaggccggtgcctacctccaccatccaccccacaacacgcgaaacacatattcccacgaccatgacaaccagtcatgataccgattctaaccggccgaatcccattgacatctccgagtcgacagaacccgggccattaactaacaccactcgcggagcagcaaatttgctgacaggctctcgcaggacgagacgagagattactcttaggacacaggctaaatgcaaccctaacctccactattggaccacacaggacgagggcgccgctatcggattggcctggatcccctacttcggacccgctgccgagggcatttataccgaaggaattatgcacaatcagaacggtctgatttgtggtctaaggcaactggctaacgaaacaactcaggcgcttcaactcttcctcagagcgaccaccgaactgaggaccttcagtatcctcaacaggaaggccatagactttttactccagagatggggtggaacatgtcatattctgggacccgattgttgtatagagccacacgactggacgaaaaatatcacagataaaatagatcagattatccatgacttcatagacaagcctctgcccgaccagaccgacaacgataactggtggactggatggcgccaatgggtccccgctggcataggaattactggtgtgattatagctgtcatcgccctgctatgtatctgcaaattcttactctaa

SEQ ID NO: 170 - RNA, 2031 nt, synthetic construct
  misc_feature 1..2031: note = SUDV GP, Gulu, Uganda 2007, optimized mRNA Sequence
  source 1..2031: mol_type = other RNA
atgggtgggttatctctgttgcaactccctagagacaagttcaggaagtcgtctttcttcgtatgggtcatcatactgtttcagaaagcttttagtatgccactgggcgtagtgaccaactctactctggaggtgacagaaattgaccagttggtctgcaaagaccacttagctagtaccgatcagctgaagagtgtgggtttgaatctagaaggcagtggggtgtccaccgatatcccttctgcaaccaaacgctgggggtttcggagtggagtgccccccaaggttgtttcatatgaagctggcgagtgggcggagaattgttataacctagaaatcaagaaaccagatggctctgagtgcctgccaccccctcctgatggtgtgagaggattcccacggtgcaggtatgtgcataaagcccagggaaccgggccatgtcctggtgattacgcttttcacaaggatggagcgttttttctatacgatcggcttgcatccaccgtgatctaccgaggcgtaaactttgccgagggcgtaattgctttccttatcttagccaaaccgaaggagaccttcttgcagtctcctcctatcagagaggccgttaattatacagaaaatacatccagctactacgccacttcttatctggagtatgagatcgagaatttcggggcccagcatagcaccacactctttaagatcgataataacacatttgtgagactggatcgtcctcacacaccccagtttttattccagctaaacgacactatccacctccaccagcagcttagcaataccacagggagactcatctggaccctggacgcgaatattaacgccgatatcggcgagtgggctttttgggagaacaaaaaaaatctgtccgagcagctgcgaggagaggagctcagcttcgaagcgctgtccctgaacgagaccgaggatgacgacgccgccagctctagaattaccaaaggcaggatttccgatagggcaactcgaaagtactccgacctagtgcccaaaaatagcccgggaatggtgcctctgcacattcccgagggagagactaccttacccagtcagaatagtaccgaaggccggagggtgggtgtaaatacccaggagacgataacagaaacagccgctaccattataggcactaatggcaaccatatgcagatctcaacaattggtatccgcccttcttcctcgcagattccctcaagcagtcccaccacagcacccagtccggaggctcaaactcctaccacgcacacgagcgggccttcagttatggccaccgaggaacccaccacacccccggggagtagcccaggtcccaccacagaggcaccaacattgacaacccctgagaacattactaccgccgtgaagactgtgcttccccaggagtcaacctcaaatggcctaataacatccacagtaacgggcatcctggggagtcttggcctgcgaaaaaggtctaggcgtcagactaacaccaaggctacgggcaagtgtaaccccaatttgcattactggaccgctcaggaacagcataacgccgccggaatcgcctggatcccttactttggaccaggcgcagaaggaatatatactgagggcttgatgcataaccagaatgcactcgtttgcgggctccgacagctggcaaacgaaacaacacaggccttacagctattcctaagagcaaccacagagctgcgcacctacactatcttgaatcgtaaagcgattgatttcttgttacgtaggtgggggggcacgtgcaggatcttgggccccgactgttgtatcgagccgcatgactggaccaaaaatatcacggacaagatcaaccagatcatccacgactttatcgataaccctctccccaaccaggacaatgacgataattggtggaccggctggaggcagtggatccctgccggaataggcatcaccggcatcattatcgccattatcgcactgctgtgcgtgtgtaagctgttgtgttga

SEQ ID NO: 171 - RNA, 2031 nt, synthetic construct
  misc_feature 1..2031: note = TAFV GP, Cote dIvoire 1994, optimized mRNA Sequence
  source 1..2031: mol_type = other RNA
atgggagccagcgggattctacaattgcctagagagcgcttccgaaaaactagcttcttcgtctgggtgatcatcttattccacaaggtgttcagcattcctctgggtgtagtccataacaatacgttacaggtgtccgatattgataaattcgtgtgccgagataagctcagctccacttcacaactgaagtccgtggggctgaacttggaaggtaatggcgtagcgacagacgttccgactgccaccaaaaggtggggatttcgagcaggcgtgccgcccaaggtcgttaactgcgaggcaggcgaatgggccgagaactgttacaacttagccatcaagaaggtagatggcagtgagtgtttgcccgaagcccctgaaggagtgcgagatttcccccggtgcaggtatgtacacaaggtatctggtacgggaccatgtccaggtggtcttgcctttcacaaagagggggcatttttcttgtacgatagacttgcatcaaccattatctacagaggaacgacatttgcagaaggggtgattgcctttctcattctgcctaaggccagaaaagactttttccaaagtcctccattgcatgaaccggcgaacatgaccacagacccaagcagttactatcacaccactaccatcaactatgtcgtcgacaacttcggaaccaataccaccgagtttctcttccaagtcgatcacttgacatacgtgcagctggaggcaaggtttaccccgcagttccttgtgcttcttaacgagaccatatatagcgataaccgacggagcaataccactggtaaactgatctggaagatcaatccaacagttgatacctccatgggagagtgggccttctgggagaacaagaaaaatttcactaagacgttatcatccgaggaactctcattcgtacctgtccccgaaacacagaatcaggtgttggacaccacagccaccgtgtcaccccctatcagcgctcacaatcatgccgccgaggaccacaaagaattggtgtcagaggactctacccccgttgtccagatgcagaatatcaaaggaaaggacaccatgcctacaactgtgactggggtaccgacgacaactccatctccgttccctatcaacgctagaaacactgatcataccaagtcgttcattgggctggaaggaccacaggaggatcatagtaccactcagcctgctaagacaacctctcagcccacaaactcgactgagtctaccactctgaaccctaccagcgagcctagctcacggggtactgggcctagctctcccaccgtgccaaacacaactgaatctcacgctgagctgggaaagacaaccccgaccactctgcccgagcaacatactgccgcttccgctattcccagagctgtccatccagatgagctgtccggacccggttttctcacaaacaccattaggggtgtgacaaatctgctgacagggtctaggcgtaagagacgcgacgtgacacccaatacccagccgaagtgcaatcccaacttacactactggacagccctggatgagggcgcggccatcgggctagcttggatcccttattttggccccgcagctgaagggatctacactgaaggaataatggagaaccagaatgggctcatctgcgggttgagacagctcgctaacgagactacccaggccttgcagctgttcctgcgggcgactactgagcttagaacctttagcatcctcaatcgcaaagccatcgacttcctgctgcagcgttggggcggaacttgtcacatactgggcccagactgttgcattgaacctcaggactggacaaagaacattactgacaagatagatcagatcatccacgactttgtggacaacaatctgcctaatcagaatgatgggtcaaattggtggacaggttggaagcaatgggtccctgctgggatcggcattactggtgtaatcatagctattatcgctctcttgtgcatctgtaaattcatgctgtga

SEQ ID NO: 172 - RNA, 981 nt, synthetic construct
  misc_feature 1..981: note = BDBV VP40, Uganda 2007, optimized mRNA Sequence
  source 1..981: mol_type = other RNA
atgaggagggcaatacttccgaccgctcccccagaatatattgaagccgtgtatccaatgaggactgttagtaccagcatcaatagcacagcgtcgggaccaaatttccctgccccggatgtgatgatgagcgacaccccctcaaactctctaagacctatcgctgacgacaacatcgaccatccatcccacaccccaacctcagtttccagtgcattcatactggaggccatggtcaatgtaataagcgggccaaaagtactcatgaaacaaattccaatttggcttccgctcggggtggcagaccagaagacctactcatttgacagcactaccgcagctattatgttagcaagctacacgataacgcactttgggaaaacttccaacccgttagtaagaatcaatcggcttggtcccggtattccggaccatcctcttcgactgttgcgcatagggaatcaggctttcctacaagaatttgttcttccaccagtgcagcttcctcagtactttactttcgatctcacagccctgaagctaatcacccagccattgccagcagcaacatggactgacgatactccaactgggccgaccgggatcctgcgtcctgggatctcttttcatccaaagttacgacccatactcttgccaggtaagacaggcaagaggggctcatcttccgatctcacttctcctgataaaatacaggccattatgaattttctgcaggacctgaagctggtacctatcgatcccgctaaaaacatcatgggtattgaggtccctgagctactcgtgcataggttaaccgggaaaaaaattacaactaaaaatggccaaccaatcatccctatcctcctgcctaaatacatcggaatggaccccatctcacagggcgacctgactatggttatcacccaagactgcgacacttgccatagtcccgctagcctccctcccgtcagcgaaaaatga

SEQ ID NO: 173 - RNA, 981 nt, synthetic construct
  misc_feature 1..981: note = SUDV VP40, Gulu, Uganda 2000, optimized mRNA Sequence
  source 1..981: mol_type = other RNA
atgaggagagtgacagtaccaaccgcccctccggcttacgccgatatcgggtatccaatgtcaatgcttcccatcaagagcagcagggcggtctcgggaatacaacagaagcaggaggtgctgccaggtatggataccccttccaacagcatgcgtcctgtggccgacgataatatcgatcatacaagtcacacaccaaatggcgtggcttctgcattcattctcgaagccaccgtaaacgtcataagcggccccaaggttctcatgaagcagattcctatttggctgccgctcggtatcgcagatcagaagacttactctttcgattcgaccaccgccgccataatgctggcttcatacaccataacacactttgggaaagctaataatccacttgtaagggtgaacagattaggtcagggaatccccgatcaccccctgaggctcctcagaatgggcaaccaggccttcctgcaggaatttgttcttccacctgtccaactgcctcagtacttcacatttgacctgaccgcactgaagttggtaacacagcctcttccggccgctacctggaccgacgagactccatctaatctctccggagctttgcggcctggtttgagtttccacccaaaactccggcccgtactgctccctgggaagacaggcaaaaagggccacgtgtcggatctgactgcccctgacaagattcagaccatagtgaacctgatgcaggacttcaaaatagtgccaatcgatcctgctaagtccattatcgggatcgaagttccagaactgctcgtacacaagctcaccggaaaaaaaatgagccagaaaaacggccagcccatcatacccgtgttgctacctaaatacatcggcctggacccgatttcccccggtgacctgacaatggttatcacacctgactacgacgactgccatagtcccgcgagctgttcttacttatcggaaaagtga

SEQ ID NO: 174 - RNA, 981 nt, synthetic construct
  misc_feature 1..981: note = TAFV VP40, Cote dIvoire 1994, optimized mRNA Sequence
  source 1..981: mol_type = other RNA
atgaggcgaattatcctccctacagcacctccagagtacatggaggccgtctatcctatgaggactatgaatagcggcgctgacaacaccgcttctggccccaattatacgaccacaggggtgatgaccaatgacactcccagcaattcattgaggcccgtggctgacgataatattgaccacccctctcatacacccaatagcgtggcgagtgccttcattctggaagctatggtgaatgtgatatctggcccaaaagtgctgatgaagcagattcctatatggctgcccctcggggtctcagaccagaagacctacagcttcgacagcacaaccgcagctatcatgctcgcttcctacactattactcatttcggaaaaaccagcaacccactcgtccggattaaccgccttggaccaggtatccctgaccacccacttaggctgctaaggataggaaatcaagccttccttcaggagtttgtgctgccccctgtccagttgccccaatacttcacctttgatctgacagcactgaagcttatcacccagcccctgcccgctgcgacctggactgacgagacaccggcagtctctaccggcactttaagacctggcatcagctttcaccccaagctgaggcccatcctgctgccaggaagggctggaaagaagggcagcaactcagacctgacatctccagacaaaattcaagcgatcatgaatttcctgcaggatcttaagatagtgcccatagatcctacaaagaatatcatggggatcgaagtgcccgaattgctggtacatagattaactgggaaaaagactaccactaaaaatgggcagcctattatccctatactgctgcctaagtatatcggcctggaccccctgtctcagggcgatttaactatggtgatcacccaggactgcgactcttgccacagtccggcatcactgccgcccgttaatgagaaatga

SEQ ID NO: 175 - RNA, 2088 nt, synthetic construct
  misc_feature 1..2088: note = MARV NP, Angola 2005, optimized mRNA Sequence
  source 1..2088: mol_type = other RNA
atggacttgcattctctgttagaactcggtacaaagcctaccgccccacacgtgcgtaataagaaagtcatccttttcgatactaaccatcaggtcagcatatgtaatcagatcattgacgccattaacagtggaattgaccttggtgacctcctcgagggaggcttactcaccctttgtgtggaacactactacaactcggacaaagacaagttcaacacctcccccatcgcaaagtacctccgcgacgctgggtatgaatttgacgtaatcaaaaacgcagacgccacacggtttctggatgtgatccctaatgagccacactactcaccgttgatcctggccctcaaaacactggagtctactgaaagtcagaggggcagaattggcctcttcttaagcttctgttcactcttcttgccaaagcttgtcgtgggagacagggccagtatcgagaaggcattgagacaggtcaccgtgcaccaggagcagggcattgtgacgtaccccaatcattggctcacaaccggccacatgaaggtcatatttggtatacttaggtcatcgtttatccttaagtttgtgcttattcaccagggagtgaacctggtgacgggacatgatgcttacgactccatcatatccaattctgtcgggcagacccggttctctggcctgctcatcgtgaagaccgtgcttgaatttattctgcagaagacggattccggcgtgactctgcatcccctggttcgcacctcaaaagtaaagaatgaggtggccagtttcaaacaggctctatccaaccttgcaaggcacggggaatatgctccgttcgccagggtgctgaatttgagcggaattaacaacctggaacatggactatatccccaactctcagccattgccttaggcgtagccaccgcccacgggtccaccctggccggagtcaatgtgggcgagcagtaccaacaattgcgggaggctgcgcatgacgctgaggtgaaactgcaaagacggcatgagcaccaggaaattcaggccatcgctgaggatgacgaagagagaaagattctcgagcagttccatttacagaaaaccgagattactcattcccagactttagcggtactctcgcaaaagcgggagaaactggctcgcttggctgccgagatagaaaataatattgtcgaggatcagggctttaaacaatcacaaaatagggtaagtcaatcttttctcaacgaccccacaccggtggaagtgaccgtccaagcccggcccattaaccgccccactgctctgccccctccagtagactcaaagatagagcatgaatcaaccgaagattcttcctccagtagctcctttgtcgaccttaacgacccattcgctctcctcaatgaagacgaagacaccttagacgactcagtgatgatcccctccacaaccagcagagagtttcagggaatcccggagcctccaaggcagtcccaagatattgataacagccagggcaaacaggaagatgaaagtaccaatttgatcaagaaacccttcctgcgttaccaggaactgccccctgttcaggaagatgacgaatctgaatacaccactgatagccaggagtcaatcgaccagccaggttcagacaacgagcagggcgtggacctgcctcctccccccctctatgcccaggagaaacgtcaggacccaatccaacatcctgctgttagctcccaggatccttttggatcaattggagatgttaacggagacattctggaaccaatccggagtccctcttcaccatccgcacctcaggaggacacacgggccagagaggcgtatgagctgtccccagatttcaccaactacgaggataatcagcagaactggccccagcgcgttgtgacaaaaaagggcagaacgttcctttaccctaatgaccttctgcagactaatccccctgagtcgctcatcaccgccctagtggaagagtaccagaatccggtgagtgccaaggagctgcaggcagactggccggacatgtcctttgatgaaagaaggcacgtcgccatgaacttataa

SEQ ID NO: 176 - RNA, 2220 nt, synthetic construct
  misc_feature 1..2220: note = BDBV NP, Uganda 2007, optimized mRNA Sequence
  source 1..2220: mol_type = other RNA
atggatcctagacccattcgaacgtggatgatgcacaatacatctgaagtggaagccgactaccataaaattctgactgcagggttgtcagtccaacagggcatcgtccgacaacgcattatcccagtgtaccagataagcaatcttgaggaggtgtgccaattgatcattcaggcctttgaggcaggcgtcgactttcaggattctgccgacagttttctcctcatgctctgtttgcaccacgcttaccagggtgactacaaacagtttctcgaaagcaacgccgtgaagtatcttgagggacacggatttcggtttgagatgaagaaaaaggaaggcgtgaaacggctcgaggagcttttacctgcagctagttccgggaagaatattaaacgtactctggctgccatgccggaggaagaaactacagaagcaaatgcaggccagtttctcagctttgcttctttgttcctgcccaagttggtggtgggcgagaaggcatgcctggaaaaagtgcagaggcaaattcaagtgcatgcggaacaggggctaatacagtaccccacgtcctggcaatccgtcggccatatgatggtgatttttcgcctgatgcgcactaattttttgatcaaatttctcttgatacatcaaggaatgcacatggttgcgggtcatgatgctaacgatgctgtaattgccaattcagtggcccaggcaagattcagcggtttattgatagtaaaaaccgtcttggatcacattctccagaaaacagaacacggggtgcggcttcatcctctggcaaggacagccaaggtgaagaacgaggtgagcagctttaaagctgccctggcttctctcgcccagcacggggagtatgccccgttcgccaggctcctcaacttgtctggcgtgaacaatctcgagcatggactgtttccccagctctccgcaatagctctcggggtggcaaccgcacatggttcgacactggctggggtgaacgtgggtgagcaataccagcagcttcgcgaagccgccacagaagccgagaagcagctgcagaaatatgcagaaagcagagagttggaccacctgggcctggacgaccaagaaaagaagatcttaaaagatttccatcagaagaagaatgaaatctctttccaacagaccacggctatggtgaccctccgtaaagagagactcgccaagctgacagaggcgataacctccaccagcattctgaaaacagggagaaggtatgatgacgataacgacatacccttccctggccccatcaacgacaatgagaattcaggccagaacgacgacgatcccacagactcccaggacaccacaatcccagacgtcatcatagacccaaatgatggcgggtacaacaattattccgactacgctaatgatgctgcgtctgctcctgatgacctggttctattcgacctggaagacgaggacgacgcagataaccccgctcaaaacaccccagagaagaatgatagacctgctacaaccaagctacgtaacggtcaggatcaggacgggaatcagggagaaacggccagtcctcgggtggcccctaatcagtatcgagacaaacccatgccccaagtgcaggaccgttctgagaaccacgaccagaccctccagacccagtctcgggtcctgacgcccataagcgaggaagccgacccaagcgatcacaacgacggcgacaacgaaagtatccctcctctggagtcagatgatgaggggagtaccgacactaccgcagccgagaccaaacccgccaccgcccctcccgcacctgtttataggagtattagcgtggacgatagcgttccgagtgagaatatcccggcccagtcaaatcagacaaacaatgaggacaatgtgagaaataacgcacaaagtgaacagtccatcgccgaaatgtatcagcacatcctcaagactcagggcccctttgatgcgattctctactaccatatgatgaaagaggagccgatcatattttcaacttccgatggcaaagaatatacctaccctgatagcctcgaggatgagtaccccccctggctgtccgaaaaagaggctatgaatgaggataaccgcttcattacaatggatggacagcaattctactggcctgttatgaaccacagaaataaattcatggccatattgcagcatcacagatga

SEQ ID NO: 177 - RNA, 2217 nt, synthetic construct
  misc_feature 1..2217: note = SUDV NP, Gulu, Uganda 2000,optimized mRNA Sequence
  source 1..2217: mol_type = other RNA
atggataagagggttcgtgggagctgggccctgggaggccagtctgaagtcgatcttgactaccacaagattttaactgccggtctgtccgtacagcagggtattgtccgccagcgcgtcatccctgtgtacgtggtttctgatcttgaagggatatgccagcatattatccaggctttcgaggccggggttgactttcaggacaacgccgattcgttccttctcctgctctgtctgcaccacgcataccagggcgaccacaggttgttcctcaagtccgatgctgttcaatacctggaaggccatgggttccgatttgaggtgcgggaaaaagaaaatgtgcaccggctggacgaactgctcccaaacgtgactggggggaaaaacctgcggagaacattggccgctatgccggaggaggaaaccactgaggccaacgctgggcagtttctgtcttttgccagccttttcctgcccaaacttgtggtgggtgagaaagcttgtcttgaaaaagtgcagcgccaaatccaagttcacgccgagcagggactgattcagtatcccacttcatggcagtccgtcggccatatgatggtaatcttccggctcatgagaaccaacttcctgatcaaatttctgctgattcaccaaggtatgcatatggttgccgggcacgatgcgaacgacaccgtgatcagcaattcagtggcccaggctcgcttctccggcctgttgatcgtgaaaaccgttctggatcatatcctgcagaaaacagacttgggcgtacggttgcaccctctggctcggacggccaaagtcaagaacgaggtatcaagcttcaaggcagcgctgggtagtctagccaagcatggagagtacgctccgtttgctagactcctcaacctgagtggtgtgaacaaccttgaacacggcctgtatcctcagcttagcgctatcgccctcggcgttgctaccgctcacggaagcacgttagccggggtgaacgtgggcgaacagtatcagcaattgagggaagcagctaccgaggcggagaaacagctgcagcagtatgcagaaacaagggaacttgacaatctgggcctcgacgaacaggagaagaagatcctgatgagttttcaccagaaaaagaacgagatttcgttccaacagacaaatgctatggtgactctgaggaaagagcgcttggccaagttgacagaggcaatcacaaccgctagcaaaataaaggtaggcgacagatatcccgacgataatgatattccttttccagggcccatctatgatgagacgcacccaaatccaagcgatgacaatcccgatgacagcagggatactaccattcccgggggagtggttgacccctatgatgatgaaagtaataattacccagactatgaggatagtgcagaggggaccaccggggacctcgatttgttcaatctggatgatgacgacgatgatagccagccagggcctcctgacaggggccagtctaaggaacgggcagccaggacacacggcttacaggatcctaccctcgacggcgctaaaaaagttccagagttgactcctgggagccatcaaccgggcaatctccatatcaccaagcccggaagcaatacaaatcagcctcagggtaacatgtccagtacattgcagagtatgacccccatccaggaggagagcgaaccggacgatcagaaagatgacgatgacgagtcccttacatccctggactcggagggtgacgaagacgtggagtccgtatcgggggaaaataaccctactgtcgctcctcccgcacccgtatacaaggacaccggtgtagacacaaaccaacagaatgggccctccaatgcagtggacggtcaaggatccgaatctgaagccctccctataaacccagagaagggcagtgctcttgaggaaacctactatcatcttctgaaaactcagggtcctttcgaggccattaactactaccatttaatgtccgacgaaccaattgcattctcaactgaaagcggtaaggaatatatttttcccgactcactggaggaagcctaccctccctggctctccgagaaggaagccctcgagaaagagaatagatacctggtgatagatggccaacagttcctgtggccagtcatgtctttacaagataaattcctggcagtcttacagcacgactga

SEQ ID NO: 178 - RNA, 2220 nt, synthetic construct
  misc_feature 1..2220: note = TAFV NP, Cote dIvoire 1994,optimized mRNA Sequence
  source 1..2220: mol_type = other RNA
atggagagtcgggcacacaaggcctggatgacccatacagccagtggattcgagacagattaccataagatcttgaccgccggcttgagtgtacagcagggcattgtcaggcagagggtgatccaggtgcatcaggtgacgaaccttgaggagatttgccagctgattattcaggcctttgaggcaggggttgattttcaggagtctgctgacagtttcctgttgatgctctgtttgcaccacgcataccagggcgattacaagcagttcctggagagtaacgctgtgaagtatctggagggacacggatttcgcttcgaggtgagaaagaaagaaggagtgaagcgtctcgaggagctgcttcccgcagcttctagtgggaaatctatcaggagaactctcgcggccatgccagaggaggaaactactgaagctaatgctggccagttcttgagctttgcaagccttttcctgcccaagctcgtcgtcggcgagaaagcatgcctggagaaggtccagcgccagatccaagtacattcggaacagggcctcattcaatacccaactgcttggcagtccgtggggcatatgatggttatatttaggcttatgcgtacaaactttttgatcaagtttctgttgatccatcagggcatgcacatggttgcagggcacgacgcaaacgatgcggtgattgcaaattctgtggcccaggctagatttagcggtttgttgatcgtcaagactgttttagaccatatactccaaaagaccgagcacggcgtgaggttgcacccgttggccagaactgctaaggttaaaaacgaagtgaatagctttaaagcagcactcagttccctcgctcagcacggggagtatgccccgttcgcccgtttgcttaatctttctggcgtcaacaaccttgaacacggcctctttcctcagttatcagccattgcgctgggggtggctacagcacacggcagtactctcgcgggcgtaaatgtgggcgaacagtaccagcagttgagggaggcggcgacagaagctgagaaacaactacagaagtatgctgagagccgcgaactggatcaccttggtcttgatgaccaggagaaaaaaatcctgaaagacttccaccagaaaaagaacgaaatatcatttcaacagaccaccgcaatggtgaccctcagaaaggagagactggccaagctgaccgaggcaatcacttccacctcgttacttaagactggaaagcaatatgatgatgacaatgatattccgttccctggaccaataaatgacaacgagaatagtgagcagcaggacgatgatcctaccgatagccaggatacgacaatcccagatatcatagtggaccctgacgatgggaggtacaacaactatggcgattaccccagcgagaccgctaatgcccctgaggacttagtcttgttcgatctggaggatggagacgaggacgaccacaggccctcatctagttcagagaataacaataagcactcactgacaggtaccgactccaataaaacatccaattggaacagaaacccaaccaacatgccaaaaaaggacagcacccagaacaacgataatccagcgcagagggcccaggaatacgctcgtgataatattcaggacacgccaaccccacatcgcgctctgacacctatttccgaggagaccgggtcaaatgggcacaacgaagacgatatcgactcaatcccacccctcgagtctgacgaggaaaacaacactgagactaccataacaacaaccaagaataccaccgcacctccagccccagtctatcgcagcaattcagaaaaggagcctctcccacaggaaaagtcacagaaacagcctaatcaggtgtcaggctccgaaaatactgacaataaacctcattccgaacagagtgttgaagagatgtacagacacatattgcagactcaggggccctttgacgcaattctgtattactatatgatgaccgaagagccaattgtattttccacatctgacggaaaggaatacgtgtatccagattctttggagggggagcacccaccttggctgagcgaaaaagaggctttaaatgaggataatcgctttattacaatggatgatcaacagttttattggcctgtgatgaatcaccgaaataagtttatggctatcttgcaacaccataagtaa

SEQ ID NO: 179 - RNA, 2031 nt, synthetic construct
  misc_feature 1..2031: note = EBOV GP, Mayinga, Zaire 1976, optimized mRNA Sequence
  source 1..2031: mol_type = other RNA
atgggcgttacaggcatcctacaactcccccgcgatagattcaaaagaacttccttctttctctgggttataatcctattccaacgaaccttcagcatacccttgggagtgatccacaactcaacactccaagtttccgacgtcgacaaacttgtatgccgagacaaactttcctcaacaaaccaattacgctcagtcggtctaaacctagaaggcaacggagtcgcaacggacgttccatcagcaaccaaacgatggggattcagatccggagtcccacccaaagtagtcaactacgaagcaggcgaatgggccgaaaactgctacaacctagaaatcaaaaagccagatggatcagagtgtctaccagcagcaccggatggaattagaggtttcccaagatgccgatacgtccacaaagtatcaggaacaggaccatgtgctggagacttcgcgttccacaaagaaggcgcattcttcctttacgatcgacttgcctcaacagtaatatacagaggcaccacctttgcagaaggcgtagtcgcattcctgatcctcccacaagccaagaaagattttttctcctcccaccctctccgagaaccagtgaacgccacagaagacccaagttccggatattattccaccacaatacgataccaagccacaggattcggaaccaacgaaaccgaatacttattcgaagtagataacttaacctatgttcaattagaatctcgctttacaccccaatttctacttcaattaaacgaaacaatatatacatctggaaaacgctcaaatactacaggaaaactcatatggaaagttaacccagaaatagacaccaccataggcgaatgggctttctgggagacaaaaaaaaacctcacacgcaaaatacggagcgaagaactatcattcacggtcgtaagcaatggagcaaaaaacatctcaggccaaagtcccgccagaacatcatcagacccaggaaccaatactactacagaagaccacaaaataatggcctcagaaaactcctcagctatggtccaagtccactctcaaggtcgtgaagccgctgtaagtcacctcacaactctagccaccatatctacttccccacaatccctaacaactaaaccaggccccgataactctacccacaacactcccgtatataaactagacatctctgaagccactcaagtagaacaacaccacagaagaacagacaatgacagcaccgcctccgataccccatcagctacaactgctgccggcccacccaaagctgaaaatacaaacacatcgaaatccactgacttcctagatccagccacaaccacttccccccaaaaccatagcgaaaccgcaggcaacaacaatacacaccaccaagatactggagaggaatccgcaagcagcggaaagctcggcctaatcacaaatacaatcgcaggcgtagccggactcatcacaggtggcagaagaacaagaagagaagccatcgtcaacgcacagccaaaatgcaaccctaacctccactactggacaacgcaagacgaaggagccgccataggtttagcatggatcccgtacttcggcccagcagcagaaggaatctacatcgaaggcctcatgcacaaccaagacggacttatctgcggcctacgccaactcgctaacgaaaccactcaagcactacaactattcctacgcgccaccacagaactacgcaccttctccatactaaacagaaaagcaatcgacttcctcctacaaagatggggtggcacatgccacatcctcggaccagactgctgcatagaaccacatgactggacaaagaatatcacagacaagatagaccaaataattcacgactttgtagacaagacactgccagaccaaggagataatgacaactggtggaccggctggcgacaatggatccccgcaggcatcggcgttaccggagttataatcgccgtaatcgcactcttctgcatctgcaaatttgtattctaa

SEQ ID NO: 180 - RNA, 2031 nt, synthetic construct
  misc_feature 1..2031: note = EBOV GP, Sierra Leone 2014, optimized mRNA Sequence
  source 1..2031: mol_type = other RNA
atgggtgttaccggaatattacaacttcccagggacagatttaaaagaacatccttcttcctatgggtgatcatcttattccagcgcacctttagcattccactaggagtcatacacaactctaccctacaagtcagcgacgttgacaaactagtatgtagagacaaactctcatcgacaaatcaactacgctcagtaggattaaacctagaaggaaacggagtagcaacagacgtaccatcagtgacaaaaagatgggggttcaggtcaggcgtccccccaaaagttgtcaactatgaagccggcgaatgggcagaaaactgctacaacttagaaataaaaaaaccagacggatctgagtgcctcccagcggcaccagacggaatacgtggattccccagatgccgatacgttcacaaagtaagcgggacaggaccctgcgcaggcgacttcgccttccacaaagaaggggcattcttcctatatgaccgactagcatcaaccgtaatatacagaggtaccacattcgcagaaggagtcgtcgccttcctcatcttaccacaggctaaaaaagacttcttctcaagtcacccactacgagagcccgtaaacgctaccgaagacccatctagtggctattactcaacaaccatcagataccaagccacaggcttcggaacaaacgaaaccgaatatttatttgaagtagacaacttaacatatgtacagctagaaagtagattcaccccccaattcctgctccaattaaacgaaacaatatatgcatctggcaagagatccaacaccaccgggaaactaatttggaaagtaaacccagaaatcgacaccactatcggagaatgggcattctgggaaacaaaaaaaaaccttacaaggaaaatccgctccgaggaactatcattcacagcagtatctaacggaccaaaaaacatctcaggccaatcaccagcccgcacatcatccgatcccgaaacaaacacaacaaacgaagaccacaaaataatggcaagcgaaaacagcagtgcaatggtacaagttcactcacagggccgcaaagctgcagtatcccacctgacaaccctagctacaatctcaacctctccgcaaccacccacaaccaagactggaccggacaatagtacacacaacacacctgtctacaaacttgacatctcagaagcaacccaggtcggacaacaccaccgcagagccgacaatgactcaaccgcctccgacaccccccccgcaacaacagcagcaggacctttaaaagctgaaaacaccaacaccagcaaaagcgctgactcccttgacctggcgacaacaacctcccctcaaaactacagtgaaacagccggcaataacaatacacaccatcaagacacaggggaggaaagcgcgagttcaggaaaactaggacttattacaaataccattgccggagtcgccggcttaataaccggcggcagacgcacccgtagagaagttatagtaaacgctcaacccaaatgcaacccaaacctgcattactggactactcaagacgaaggcgccgcaataggcttagcctggatcccgtactttggacccgccgcggaaggcatatataccgaaggacttatgcacaaccaagacggacttatctgcggactaagacagctagccaacgaaactacccaagcactacaactatttctccgcgcaaccacagaactacggacattttcaatactcaacagaaaagcaatagacttcctactccaacgctggggtgggacatgccatattctcggtcccgattgctgtatagagccacacgattggaccaaaaacataaccgacaaaatagaccaaataatccacgatttcgtagacaaaacactccccgaccaaggagacaacgacaattggtggacaggctggcggcaatggatccccgcaggaatcggagttaccggcgtcataatagccgttatcgccctattctgcatatgcaaattcgtattttaa

SEQ ID NO: 181 - RNA, 2046 nt, synthetic construct
  misc_feature 1..2046: note = MARV GP, Angola 2005, optimized mRNA Sequence
  source 1..2046: mol_type = other RNA
atgaaaaccacatgcctactaatctccctcatcctaatccaaggagtaaaaacgctaccaatactagaaatagcttcaaacatacaaccacaaaatgttgatagcgtgtgttcaggaacattgcagaaaacagaagacgtacatctcatgggattcaccctatcaggccaaaaagttgctgattcaccactagaagccagcaaaagatgggcattcagagccggtgtccctcctaaaaacgtagaatacaccgaaggagaagaggcaaaaacctgctacaatatctccgtaactgacccatcaggaaaatccctactcttagacccacccacaaatatacgcgactaccccaaatgtaaaacaattcaccacattcaaggacaaaacccccacgcacaaggaatagcattacatctctggggcgcattcttcctctacgatagaatcgcatcgactaccatgtaccgtggcaaagtcttcacagaaggaaatatcgctgccatgatagtcaacaaaactgtccacaaaatgatattctccagacaaggtcaaggttacagacatatgaatctaacatcaactaacaaatactggacatcaagcaatggtacacaaacaaacgacacaggatgcttcggcactttacaggaatacaactcaaccaagaaccaaacatgcgccccatccaaaaaaccacttcccctcccaaccgcccatcctgaagtcaaactaacctccacatccactgacgcaactaaactgaacactacagatcccaactcagacgacgaagatttaactacctccggctccggaagcggagaacaagaaccatacaccacctcagacgcagccacaaaacaaggcttgagctcaactatgccacctactccgtctccacaaccaagcacaccccaacaaggaggaaataacacaaaccattcacaaggcgttgtcacagaacccggtaaaaccaacacaaccgcacaaccctcaatgcctcctcataacactacgactatatcaactaataatacctccaagcacaacctatcaacaccatcagtcccaatacagaacgccacaaactataacacacaaagcactgcccctgaaaacgaacaaacttccgcaccatccaaaacaactctattaccaacagaaaatccaacaacggccaaatcaacaaactcaaccaaatctcccaccacaacagtaccgaatactacaaataaatactcaacctcaccaagcccaacacccaattctacagcacaacatttagtctacttcagacgaaaacgcaatatcctatggagagaaggagacatgtttcccttcttagatggactaataaacgcacccattgatttcgacccagtaccaaacactaaaacaatattcgacgaaagttcatcctccggagcttcagcagaagaggatcaacacgctagccccaacattagtctcactctaagttatttcccaaaagtcaatgaaaataccgcacattccggcgaaaatgaaaacgactgcgacgcagaactccggatttggtcagtacaagaagacgacctagctgcaggactttcatggataccattttttggccccggaatcgaaggcttatataccgccggattaattaaaaaccaaaacaatttagtctgccggctacgcagactagcgaaccaaacagccaaatccctagaacttttactacgagttaccacagaagaaagaacattctcactaatcaatagacacgccatagacttcctactagcgcgatggggcggaacctgcaaagtattaggccctgactgttgtattggaattgaagacctatctaggaacatatccgaacagatagaccaaataaaaaaggacgaacagaaagaaggcacaggctggggcctaggtggaaaatggtggaccagcgactggggtgttctaacaaatctaggcatcctactcctcctatcaatagccgtactaatagcactttcctgtatctgtcgaatttttacaaaatatataggctaa

SEQ ID NO: 182 - RNA, 981 nt, synthetic construct
  misc_feature 1..981: note = EBOV VP40, Mayinga, Zaire 1976, optimized mRNA Sequence
  source 1..981: mol_type = other RNA
atgcgtcgcgtcatactccccaccgcaccccccgaatacatggaagccatataccccgttcgttccaactcaaccatagcacgaggaggaaattcaaacaccggattcctcacacccgaatctgtcaacggagatacaccttctaacccactaagaccaatcgccgacgacactatagatcacgcatcccacacccccggctcagtttcctccgcatttatcctagaagctatggtaaacgtcatctccggacctaaagtactcatgaaacaaatccccatatggctcccccttggagtcgcagatcaaaaaacctactcattcgactcaacaacagcagcaatcatgctagcctcttataccataacccacttcggaaaagcaacaaatccacttgtacgcgtaaaccgattaggaccaggcatacctgaccacccactcagacttctccgcataggcaaccaagccttcctccaagaattcgtactaccccccgtccaacttccacaatacttcaccttcgacctaactgcactaaaactcataacccaacccctaccagcagccacctggacagacgacaccccaaccggctcaaatggagccctacgccccggaatctcattccacccaaaattacggccaatcctcctaccaaacaaatcaggcaaaaaaggaaattccgccgacctaacatcccctgaaaaaatccaagccatcatgacttcattacaagactttaaaatagtaccaatagacccaacaaaaaacatcatgggcatagaagtcccagaaaccctagtacacaaactaaccggaaagaaagttacatccaaaaacggacaaccaataatacccgtcctactaccaaaatatatcggtttagaccccgtcgcaccaggagacctaaccatggtcatcacccaagactgcgacacatgtcactcacctgcatccctaccagccgtcatcgaaaagtaa

SEQ ID NO: 183 - RNA, 981 nt, synthetic construct
  misc_feature 1..981: note = EBOV VP40, Sierra Leone 2014,optimized mRNA Sequence
  source 1..981: mol_type = other RNA
atgcgccgagtcatcctaccaaccgcacctccagaatacatggaagctatataccccgcccgatccaactccactattgcacgaggcggtaactcaaacaccggtttcctaactcctgaatccgtaaacggcgatacaccctccaacccactacgcccaatagccgatgacacaatcgatcacgcatcacatacaccaggctcagtatcatccgccttcatactcgaagcaatggtcaacgtcatatcaggcccaaaagtcctaatgaaacaaatacccatttggttacctctaggcgtagccgaccaaaaaacatactcattcgactcaaccacagccgcaatcatgctcgcatcatacactatcacccacttcggcaaagcaaccaacccactagtcagagtcaacagactcggcccaggaatcccagatcacccactacgactcctacgaatcggcaaccaagcctttcttcaagaatttgtcctaccaccagtccaactaccccaatacttcaccttcgacttaaccgccctaaaactcataacccaaccacttccagccgcaacctggacagacgatactcctactggatccaacggtgccctccgaccaggaatcagctttcacccaaaactcagacccatcctattaccaaacaaatcaggcaaaaaaggaaactcagcagacctcacatcccccgaaaaaatccaagccataatgacctcactacaagactttaaaatcgtcccaatagacccaaccaaaaacatcatgggcatagaagtacctgaaactctcgtccacaaacttacaggcaaaaaagtaacatcaaaaaacggccaacccataatacccgtcctcctcccaaaatacatcggtctagaccccgtagccccaggagacctcacaatggtaataacccaagactgtgacacatgccactcccctgcatcactacccgcagttgtcgaaaaataa

SEQ ID NO: 184 - RNA, 912 nt, synthetic construct
  misc_feature 1..912: note = MARV VP40, Angola 2005,optimized mRNA Sequence
  source 1..912: mol_type = other RNA
atggcatcatcatccaactacaatacttacatgcaataccttaacccacccccctacgcagaccatggtgctaaccaactcatccccgcagatcaactctcaaaccaacaaggcatcactcccaactacgttggagacctaaatctcgacgaccaattcaaaggaaacgtatgtcatgcattcacccttgaagcaataatagatatatccgcatacaacgaaagaaccgtaaaaggagtccccgcctggctccccttaggaatcatgtcaaatttcgaatacccactagcccataccgtcgcagctctcctaacaggctcctacacaattacacaattcacccacaatggacaaaaatttgtacgagtcaatagactcggcactggtataccagcccatcccctccgaatgttaagagaaggaaaccaagccttcattcaaaacatggtcatcccacgcaacttctctaccaatcaattcacctacaatctaaccaacctagttctctcagtacaaaaactccctgacgatgcttggcgtccaagcaaagacaaactaatcggaaacacaatgcacccagcagtctcagtacaccccaacttacccccaatcgttctccccactgtcaaaaaacaagcatacagacaacacaaaaacccaaacaatggaccactcctcgcaataagcggcatcctccaccaacttcgagtagaaaaagtacccgaaaaaacatccctctttagaatatcactaccagccgatatgttctcagtcaaagaaggaatgatgaaaaaacgaggagaaaactcaccagtagtctatttccaagcaccagaaaacttccctctaaacggctttaacaaccgccaagtagtactagcatacgcaaacccaaccttatcagccgtataa

SEQ ID NO: 185 - RNA, 2220 nt, synthetic construct
  misc_feature 1..2220: note = EBOV NP, Zaire 1976, optimized mRNA Sequence
  source 1..2220: mol_type = other RNA
atggactcacgaccacaaaaaatatggatggcgccatcccttaccgaatccgacatggattaccacaaaatactaacagcaggcttatcagtccaacagggcatagtccgacaacgagtaatacccgtataccaagtcaacaacctcgaagaaatatgtcagcttatcatacaagcatttgaagcaggagtggatttccaagaatcagcagactccttcttactcatgctctgcctacatcacgcatatcaaggcgactataaattatttcttgagtcaggagcagtcaaatatttagaaggccacggattcagattcgaagttaaaaaaagagacggtgtaaaaagactcgaagaactcctaccagcagtctcaagcgggaaaaacatcaaacgaacgttagcggccatgcctgaagaagaaacaaccgaagcgaacgcgggacaatttttatctttcgcatcactattcttacccaaactggtcgtcggagaaaaagcctgcctcgaaaaagtgcaacgccaaatacaagtacacgcagaacaaggcctcatacagtaccccaccgcctggcaatccgtaggccatatgatggtaatcttccgactaatgcgaacaaacttcctgattaaattcctcctaatacaccaaggaatgcacatggtagccgggcacgacgcaaacgacgccgtcatctcaaactccgtcgcccaagccagattctccggtctcttaatcgtaaaaacagtcctcgaccatatactacaaaaaactgaaagaggagtacgattacacccccttgcgagaactgccaaggtcaaaaacgaagtcaacagcttcaaagcagcattatcatctctggcaaagcacggggaatacgcaccatttgccagattactgaacctatccggcgtcaacaacctggaacacggactattcccccaactctcagcaatagcgctaggcgtagcaacagcacacggatcaacattagcgggagtaaacgtaggagaacaataccaacaactaagagaggcagccacagaggcggaaaaacaattacaacaatacgcggaaagccgcgagctcgaccatctcggactagacgaccaggaaaaaaaaatactaatgaactttcatcaaaaaaaaaacgaaatatcctttcaacaaacaaatgccatggtaacactacgcaaagaacgccttgcaaaattaacagaggccattacagcggcatcattacccaagacatcaggacactacgacgacgatgacgatatcccctttcccggtccaataaacgacgacgacaatccaggtcatcaagacgacgacccaacagattcacaagataccacaattccagatgtagttgtagacccagacgacggaagttacggcgaataccaatcctattcagaaaacggcatgaacgctcccgacgacctagtactcttcgacctagatgaagacgacgaagatacaaaacctgttccaaatagaagtaccaaaggcggccagcaaaaaaactcccaaaaaggccaacatatcgaaggcagacaaacccaatcgagaccaatccaaaacgtcccaggaccccaccgaaccatccaccatgcatcagccccacttaccgacaacgaccgaagaaacgaacccagcggctccacatctccgcgaatgctaacaccaatcaacgaagaagcagaccccctcgacgatgctgacgatgaaacttcgagcctcccacccctggagagcgatgatgaagaacaagatcgcgacggcacatccaaccgcacccccacagtagcaccccccgcccccgtatatcgagatcattcagaaaaaaaagaactaccccaagacgaacagcaagaccaagaccacacccaagaagcacgaaatcaggactcagacaacactcagtccgaacacagcttcgaagaaatgtaccgccacatcctacgatcacaaggcccattcgacgccgtattgtactaccacatgatgaaagacgaaccagtagttttctcaaccagtgacggcaaagaatacacatacccagattcattagaagaagagtaccctccctggttaacagaaaaagaagccatgaacgaagaaaaccgattcgtcacattagatggccaacagttttactggccagtcatgaaccataaaaacaaattcatggcgatccttcaacaccaccagtaa

SEQ ID NO: 186 - RNA, 2220 nt, synthetic construct
  misc_feature 1..2220: note = EBOV NP, Sierra Leone 2014, optimized mRNA Sequence
  source 1..2220: mol_type = other RNA
atggactcacgcccccaaaaagtctggatgacaccatcgcttaccgaatcagacatggattatcacaagattctaaccgcaggattaagcgtgcaacaaggaatagtgcgccaacgagtcatcccggtctaccaggtaaacaacctggaagaaatctgtcaactcataatccaagccttcgaagcaggagtagacttccaagaatccgcagactcatttctacttatgctatgcctccaccacgcataccaaggcgactacaaactatttttagaatcaggcgctgtaaaataccttgaaggccatggattccgattcgaggtaaaaaaatgtgacggtgtaaaaagactggaagaattactccctgccgtatcctccggacgtaacatcaagcgaaccctagctgcgatgcctgaagaagagactaccgaagcaaacgctggacaattcctctccttcgccagtctattcctacccaaactagtagtcggagaaaaagcctgccttgaaaaagtccaacgccaaatccaagtacatgccgaacaaggcctaatacaataccccactgcctggcaatcagtaggccacatgatggtaatttttaggttaatgcgaaccaacttcctaatcaaatttctactaatacatcaagggatgcacatggtagccggacacgacgcgaacgacgccgtcatcagcaactcagtagctcaagcaagattctcaggtctactaattgtcaaaacagtcctcgaccatatactacaaaaaactgagcgcggagtaagacttcaccctctagcacgaacagccaaagttaaaaacgaagttaacagcttcaaagcagcactaagcagcttagcaaaacatggcgaatacgcacccttcgcaagactcctcaacctatcaggcgtaaacaacctagaacacggcctattcccacaactatcagccatcgccctcggagtcgccacagcccatggaagtacactcgcaggcgtaaacgttggagaacaataccaacaattacgagaagccgcaacagaagcagaaaagcaacttcagcaatacgctgaaagtagagaattggaccaccttggactagacgaccaagaaaaaaaaatcctaatgaacttccaccaaaaaaaaaacgaaatatccttccaacaaacgaatgccatggtcacactccgaaaagaaaggctcgctaaactcaccgaagcaatcactgccgcatcactccctaaaacctcaggtcactacgacgatgacgatgatatcccatttccaggcccaataaacgacgacgacaacccgggccaccaagacgacgatcccacagactcacaagacaccacaataccagatgtcgtcgtagaccctgacgacggaggatacggagagtaccaaagctactcggaaaacggaatgagcgccccagatgatctagtactattcgatttagacgaagacgatgaagacacaaaaccagtaccaaacaggtccaccaaaggaggacaacaaaaaaacagccagaaaggacaacacaccgaaggccgacaaacgcagtccacaccaacacaaaatgtaacaggaccacgccgtaccatccaccacgcctcagcaccattaaccgataatgaccgacgaaatgaaccatcgggttccaccagcccccgcatgttaactcccataaacgaagaagccgatcccctagacgacgccgacgacgaaacatcctccctcccacccctagaatcagacgacgaagaacaagatcgtgacggaacatcaaacagaacaccaaccgtagccccaccagccccagtctacagggaccattctgaaaaaaaagagcttccccaagatgagcaacaagatcaagaccacatacaggaagcccgcaatcaggactcagacaacactcaaccagaacactcattcgaagaaatgtaccgccacatattaagatcccaaggtcctttcgacgccgtactatactaccatatgatgaaagatgaaccagttgttttctcaacatcagacgggaaagagtacacatacccagactccttagaagaagaatacccaccctggctaacggagaaagaagcgatgaatgatgaaaaccgattcgtaactctcgacggccaacaattttactggccagttatgaaccaccgcaacaaattcatggctatattacaacaccaccaatag

SEQ ID NO: 187 - RNA, 2031 nt, synthetic construct
  misc_feature 1..2031: note = BDBV GP, Uganda 2007,optimized mRNA Sequence
  source 1..2031: mol_type = other RNA
atggtaaccagcggaatactacaactcccaagagaacgatttcgcaaaaccagcttctttgtctgggtcataatattgtttcacaaagtatttccgatcccactaggcgtcgtacataacaacacactccaggtttcagacatcgataaattagtatgccgagacaaactctcctccactagccaactaaaatccgtaggactcaaccttgaaggaaacggggtcgccaccgatgtccccaccgccacaaaacgatggggattccgagctggcgtcccacctaaagtagtcaattatgaagctggagaatgggcagaaaactgctacaaccttgacatcaaaaaagccgacggctccgaatgcctccctgaagccccagaaggcgtaaggggattccccagatgccgatacgtacacaaagtctcaggcacaggaccttgtcccgaaggctacgcattccataaagaaggcgcattcttcctatatgaccgcctggcctccaccattatatacagatccaccacattttccgaaggagtagtcgccttcctaatacttccagaaaccaaaaaagacttctttcaatctccacccctacatgaacctgcgaatatgactacagacccatccagctactaccacacagtaacactcaactatgtcgccgacaattttggaaccaacatgacgaacttcctattccaagtcgaccacctaacttacgtccaactagaaccaagatttacaccccaattccttgtccagctaaacgaaaccatatacacgaacggccgacgatccaacaccaccggcacactcatatggaaagtaaatccaactgttgacaccggagtaggagaatgggctttctgggaaaataaaaaaaacttcacaaaaacactctcgtcagaagaactctctgtcatattcgttccccgggcccaagaccctggatccaaccagaagacaaaagtaacaccaacatctttcgctaacaatcaaacatctaaaaaccacgaagacctcgtacccgaagaccccgcttccgtcgtacaagtacgcgaccttcaacgtgaaaacaccgtccccacaccaccacccgataccgtaccaaccactctaatcccagacacaatggaagaacaaacaacatcacactacgaaccaccaaatatatccaggaatcaccaagaaagaaacaacacagcccatcccgaaacactcgccaataacccacccgacaacaccaccccatcaactcctccacaagatggagaacgaacttcatcacacacaaccccaagccctcgacctgtaccaacctcaaccatacaccctaccacacgagagacccacatccctacaaccatgaccacatcacacgacaccgactcaaatcgaccgaaccctatagacatctcagaatcaaccgaacccggcccacttaccaacacaacaagaggagccgccaatctattaaccggatcacgacgtacccgcagagagattacattacgaactcaggcgaaatgcaaccccaacctccactattggaccacccaagatgaaggcgccgcaattggattagcatggataccttacttcggccccgcagccgaaggcatttacaccgaaggaataatgcacaaccaaaatggactcatctgcggattacgccaacttgcaaacgaaactacacaagccctccaacttttcctcagagccacaacagaattacgcacattctccatcctaaacagaaaagccatagacttcttacttcaaagatggggcggtacatgtcacatccttggcccagattgctgcatagaacctcacgattggacaaaaaatatcacagacaaaatcgatcaaatcatacatgattttatcgacaaacccctacctgaccaaacggacaacgacaactggtggacaggctggcgacaatgggtccccgccggcatcggaataacaggagtaataattgccgtcatagccctcctttgcatttgtaaatttctcctttga

SEQ ID NO: 188 - RNA, 2031 nt, synthetic construct
  misc_feature 1..2031: note = SUDV GP, Gulu, Uganda 2007, optimized mRNA Sequence
  source 1..2031: mol_type = other RNA
atgggagggctaagcctactacaactcccaagagataaattcagaaaatcgagtttcttcgtgtgggtcataatccttttccaaaaggccttcagcatgcccctcggagtagtcactaactctacactcgaagttacggaaattgaccaacttgtatgcaaagaccacctagcatctacagaccaactaaaatcagtcggcctcaacttagaaggttcaggagtaagcacagacatccccagtgccaccaaacgatggggattccgaagtggagtaccacctaaagtagtaagctatgaagcaggtgaatgggcagaaaattgttacaatttagaaataaaaaaacccgatggctcagaatgcctcccacccccacccgatggcgtacgcggcttcccgcgctgcagatatgttcataaagcacaaggaaccggaccatgcccaggtgactatgccttccataaagatggagcctttttcttatacgaccgcctcgcctcaacagtgatataccgcggggtaaactttgcagaaggagtaatcgcatttctcatcttagcaaaaccgaaagaaacattcctccagtcaccacccatccgcgaggcagttaattacacagaaaatacttcatcatattacgccacctcctacttagaatacgaaatagaaaactttggagcacaacactcaacgactctgttcaaaatcgataataacacttttgtacgactagacagaccacatacaccacaattcctattccaactcaatgacaccatacacctccatcaacaattatccaatacaacgggtcgactaatctggaccctagacgcaaacatcaatgcagatataggtgaatgggctttctgggaaaataaaaaaaacctatccgaacaattacggggcgaggaactatcattcgaagccctaagtctaaacgaaaccgaagacgacgacgcagcatcttcacgcataacaaaaggccgaatctccgacagagcaacaaggaaatactccgaccttgtacccaaaaattcacccgggatggtacccctccacataccagaaggcgaaacaacactaccatcacaaaactctacggaaggccgccgagtaggcgtcaacacgcaagaaaccataactgaaaccgcagcaaccatcataggcacaaatggaaaccacatgcaaatctccaccataggaataaggccatcatcaagccaaataccctcaagcagcccaaccacagccccgtccccagaagcacaaacaccaaccactcacacctcgggaccctcagtcatggcgaccgaggaacccaccacaccgcccggtagctcacctggccctactacagaagccccgacccttacaacccccgaaaacataacaactgccgtcaaaaccgtcctaccacaagaatccacttctaacggattaataacgagtacagtaacaggcatattaggttccctaggcctgcgcaaacgaagccgcagacaaactaatacaaaagccacaggaaaatgcaatccaaacctacactattggaccgcacaagaacaacacaacgccgccggcatagcctggattccatactttggacccggcgcagaaggaatatacaccgaaggattaatgcacaaccaaaacgctctcgtttgcggtcttagacaactagcaaatgaaaccacacaagccctgcaactattcttacgtgccacgactgaactaagaacatacaccatccttaaccgcaaagccatagacttcctcctcaggagatggggcgggacctgcagaatcctaggacccgattgctgcatagaaccccacgactggaccaaaaacatcaccgataaaataaaccaaatcatacatgatttcatagacaacccattgcccaaccaagacaacgacgacaactggtggacaggatggagacagtggatccccgcaggcatcggaataaccggaattataatagcaatcatagcactcctttgcgtatgcaaactcctctgttaa

SEQ ID NO: 189 - RNA, 2031 nt, synthetic construct
  misc_feature 1..2031: note = TAFV GP, Cote dIvoire 1994, optimized mRNA Sequence
  source 1..2031: mol_type = other RNA
atgggggcctctggtatccttcaattaccaagagaacgatttcgaaaaacctctttctttgtttgggtcataatactttttcacaaagtattctctatacccctaggtgttgtacacaataacaccctccaagtcagcgacatagacaaattcgtctgccgcgataaactaagctccacatcacaactaaaatccgtcggactaaacctggaaggcaacggagtagccaccgacgtacctacagcaacaaaacgatggggctttcgagccggcgtccccccaaaagtagtaaactgcgaagccggcgaatgggccgaaaattgttataacctagccataaaaaaagtagatggctcagaatgcttacccgaagcaccagaaggcgtgcgcgactttccccgatgccgatatgtacacaaagtatccggcacaggtccatgccccggtggactcgcattccacaaagaaggcgcttttttcctctatgacagattagcctccaccatcatttatcgaggcacaacattcgccgaaggagtcatcgcattcctcatcctcccaaaagcaagaaaagacttcttccaaagccctcctctacacgaacccgcaaatatgacaaccgacccctcttcatactaccacacaacaacaatcaactacgtcgtggataacttcggtactaacacgaccgaatttcttttccaagtagaccaccttacatatgttcaactagaagcgagattcaccccacaattcctagttttactcaacgaaaccatttactcagataaccgccgcagcaacaccacaggaaaactaatctggaaaataaatcccacagtagacacatccatgggcgaatgggccttctgggaaaacaaaaaaaactttacaaaaacattaagctccgaggaactcagcttcgtacccgtccccgagacacaaaatcaagttctagataccacagccacagtttcccccccaatctctgcccacaaccatgccgccgaagatcataaagaactagtatccgaagactccaccccggtcgtgcaaatgcaaaacataaaagggaaagacacaatgccaaccacagttaccggcgtcccaacaacaacgccatccccttttccaataaatgcacgaaacacagaccatacaaaaagtttcatcggcctcgaaggaccacaagaagaccactcaactacccagccagctaaaactacatcccaaccaacaaattccacagaatctacaaccctgaacccaacttcagagccatcctctcgaggtaccggaccctcaagcccaacagtccccaacactaccgaatctcacgccgaactaggaaaaaccacaccaacgaccctcccggaacagcataccgccgcatctgcaataccccgcgcagtacatcccgacgaactcagcggccccggcttcctaaccaacacaatccgaggtgtaacaaatctcttaaccggctcacgcagaaaacgacgagatgtaacccccaacactcaaccaaaatgcaaccccaacctacattactggacagccctcgacgaaggagcagcaattggactagcctggataccgtacttcggtccagctgctgaaggcatttacaccgaaggcattatggaaaaccaaaacggccttatatgcggactccgacaactcgctaacgaaacgacacaagccctccaactattcctaagagcaaccaccgaactacgcacattttcaatattaaaccgaaaggccatcgacttcttactccaaagatggggcgggacatgccacatactaggccccgattgctgtatagaaccacaagactggacaaaaaacataacagataaaatagaccaaataattcacgactttgtagataacaacttaccaaaccaaaacgacggatccaactggtggacaggatggaaacaatgggtcccagccggcataggaataacaggagtaatcatagcaatcattgcattactctgtatatgcaaattcatgctataa

SEQ ID NO: 190 - RNA, 981 nt, synthetic construct
  misc_feature 1..981: note = BDBV VP40, Uganda 2007, optimized mRNA Sequence
  source 1..981: mol_type = other RNA
atgcgacgcgcaatactccccacagctcccccagaatacatcgaagcagtataccccatgcgtacagtctcaacctccataaactcaactgcctcaggtccaaatttccccgcccccgacgtcatgatgtcagacactccttctaattcactaagacctatagcagacgacaacattgaccatccatctcataccccaacctccgtatcttctgccttcatccttgaagccatggtaaatgtcatctcaggacctaaagtactaatgaaacaaatccctatatggctccctctaggagtagcagaccaaaaaacctattctttcgactctacaacagcagccatcatgctagcatcatacacaatcacacacttcggaaaaacctccaaccccctcgtcagaatcaaccgattaggacccggaatcccagaccatccactcagattactacgtataggtaaccaagcatttctccaagaatttgtactacccccagtacaacttccacaatactttacattcgacctaaccgctctcaaactcataacacaaccactaccagcagccacatggaccgacgacacccccacaggcccaacaggaatcctacgcccaggaatctcttttcaccctaaactaagaccaatcctactaccaggaaaaactggaaaaagaggatcatcctccgacctaacatcacctgataaaatccaagctatcatgaacttcctacaagacctaaaactagtaccaatagacccagccaaaaatataatgggcatagaagtcccagaacttcttgtacaccgactcacaggaaaaaaaatcacaacaaaaaatggacaaccaatcataccaatactcctaccaaaatacatcggaatggaccccatctcccaaggcgacctaaccatggtcatcacacaagactgtgacacttgccactccccagcatccctccccccagtatcagaaaaataa

SEQ ID NO: 191 - RNA, 981 nt, synthetic construct
  misc_feature 1..981: note = SUDV VP40, Gulu, Uganda 2000, optimized mRNA Sequence
  source 1..981: mol_type = other RNA
atgcgacgagtcaccgtcccaacagcaccacccgcatacgccgacatcggatacccaatgtcaatgctcccaataaaatcttccagagcagtctcaggcatacagcaaaaacaagaagtccttcccggaatggacacaccatccaattcaatgcgccccgtagcagacgataacattgaccacacctcccacactcccaatggcgtagcctcagcattcatactcgaagctaccgtaaacgtaatatcaggaccaaaagtcctcatgaaacaaataccaatatggctccccctcggtatagccgaccaaaaaacctattcattcgattctacaaccgctgcaatcatgctagcatcatacacaatcacacactttggaaaagcaaacaaccccctagtacgcgtaaacagattaggccaaggaatacctgatcacccactacgcctcctccgcatgggtaaccaagcatttctccaagaattcgtactcccacccgtacaactaccccaatactttacattcgacctaaccgcactaaaacttgtaactcaaccactccctgcagctacctggactgacgaaaccccctctaacctctcaggcgcccttcgaccaggactatcattccaccccaaactcagacccgtcctactacccggaaaaaccggcaaaaaaggacacgtctcagacctaaccgcaccagacaaaatacaaaccatagttaacctaatgcaagacttcaaaatagtccctatagaccccgcaaaatctataataggaatagaagtacccgaactacttgtacataaactcaccggaaaaaaaatgtcacaaaaaaacggtcaacctatcatcccagtactacttccaaaatacataggattagaccccatatccccaggagatctcactatggtaatcacacccgactatgacgactgccactcaccagcatcatgctcatacctctccgaaaaataa

SEQ ID NO: 192 - RNA, 981 nt, synthetic construct
  misc_feature 1..981: note = TAFV VP40, Cote dIvoire 1994, optimized mRNA Sequence
  source 1..981: mol_type = other RNA
atgcgccgaataattctaccaacagccccccccgaatacatggaagccgtttacccaatgcgtaccatgaactccggcgcagacaacaccgcatcaggcccaaactatacaacaacaggtgttatgaccaatgacacaccctccaattccctcagaccagtagcagatgacaacatagaccacccttcacacactcccaactcagtagcatccgcattcattctagaagcaatggttaacgtaatatcaggccctaaagtactaatgaaacaaatacccatatggctaccacttggcgtctcagaccaaaaaacctattccttcgactctaccaccgccgccatcatgctcgcatcatacaccattacccacttcggaaaaacctcaaaccccctggtacgaatcaaccgcttaggaccaggaatcccagaccaccccttacgactcctccgcataggaaaccaagcattcttacaagaattcgtactaccacccgtacaattaccgcaatatttcacattcgacctcaccgccctaaaactaataactcaaccattacccgcagcaacatggacagacgaaacaccagctgtctctacaggaaccctaagaccaggcatctccttccatccaaaattacgccccatactcctccccggcagagccggaaaaaaaggttccaactcagacctaacatctccagacaaaatacaagccataatgaactttctacaagaccttaaaattgtaccaattgacccaacaaaaaatataatgggtatcgaagtccccgaactcctagtacatcgccttaccggcaaaaaaacaaccaccaaaaatggccaaccaataataccaattctacttcctaaatacataggactcgaccccttatcacaaggagacctcacaatggtcatcacacaagattgcgactcctgtcactcacctgcctcattacccccagtcaacgaaaaataa

SEQ ID NO: 193 - RNA, 2088 nt, synthetic construct
  misc_feature 1..2088: note = MARV NP, Angola 2005, optimized mRNA Sequence
  source 1..2088: mol_type = other RNA
atggatctacactctttactagaacttggcacaaaacccacagcaccccacgttcgtaacaaaaaagtcatacttttcgacacaaatcatcaagtatcaatatgtaaccaaattatagatgcaatcaatagcggaatagaccttggagacctactggaaggcggactccttacattatgcgtcgaacactactacaactctgataaagataagtttaatacatccccgatcgccaaatatttaagagacgccggatatgagttcgacgtaataaaaaatgccgatgcaaccagattcttagacgttatacccaatgaacctcactacagccctctcatcctcgcactaaaaaccctagagtcaaccgaatcccaacgaggacgcatcggactatttttatcattctgttcgcttttcctaccaaaactagtagtaggcgatcgtgcctccatagaaaaagctctccgacaagtaacagtacatcaagaacaaggcatagttacctatccaaaccactggctcacaacaggacacatgaaagtcatatttggaatactccgatcctcattcatcctaaaattcgtactaatccaccaaggggtaaacttagttaccggacacgatgcatacgatagcataatatcaaacagtgtaggacaaacacgattttcaggactactaatagtaaaaaccgtattagaatttatactccaaaaaaccgacagtggcgtcactcttcatccactagtccgaacaagcaaagtaaaaaatgaagttgcctcatttaaacaagctctcagtaatcttgctcgccacggggaatacgccccattcgcaagagttttgaacttatcaggcataaataacttagagcacgggctgtatccccaactttccgcaatagctttaggcgtagcaactgctcacggatccacattagcaggcgtaaacgtaggcgagcaatatcaacaacttagagaagcggcccacgacgccgaagtaaaactccaaagacgccatgaacaccaagagatacaagcaatagccgaggatgatgaagaacgaaaaattctagaacaatttcatttacaaaaaacagaaataacacattcccaaactctcgcagtattgtctcaaaaaagagaaaaattagcacgtcttgcagcagaaatcgaaaacaacatcgttgaagaccaaggtttcaaacagtcacaaaacagagtatcgcaatccttcctaaatgaccccacacccgtagaagtcaccgtccaagcaagacctatcaaccgaccaacagcattaccaccaccagttgactcaaaaatagaacatgaatctacagaagattcaagtagttccagcagttttgtagatctaaacgatccattcgccctccttaacgaagacgaagatacacttgacgacagcgttatgataccatctacaacatcgcgggaattccagggtattcctgagcctccaagacaatcacaagatatagacaactcccaaggcaaacaagaagacgaatccacgaacctcataaaaaagccattcctgcgctatcaagaattaccacccgtacaggaagacgatgaatcagaatacactacagattcacaagaaagtatagaccaaccgggctctgacaacgaacaaggcgtagacttaccaccccccccattatacgctcaagaaaaaagacaagacccaatacaacacccagcagtttcaagccaagaccccttcggctcaataggagacgtcaacggcgacattttagaaccaattcgatctcccagttctccgtcagccccacaagaagatacacgagcccgagaagcatacgaactgtctcccgatttcaccaattatgaagacaatcaacaaaattggccacaaagagttgttaccaaaaaaggtcgaacattcctctaccccaacgacttactacaaactaacccccccgaaagcctaattacagcactagtcgaggaataccaaaacccagtctcagcaaaagagctacaagcagattggccagacatgtcattcgatgaacgacgacacgtagccatgaacttataa

SEQ ID NO: 194 - RNA, 2220 nt, synthetic construct
  misc_feature 1..2220: note = BDBV NP, Uganda 2007, optimized mRNA Sequence
  source 1..2220: mol_type = other RNA
atggatccacgaccaataagaacgtggatgatgcacaacacatctgaagtagaagcagactatcataaaattctaacagccggactctcggtacagcaaggaatagtccgacaacgcataatcccggtctaccaaatctccaatttagaagaagtatgtcaactaatcatccaagccttcgaagctggagtcgacttccaagactcagctgattccttcctactcatgctatgcctacaccacgcataccaaggcgactataaacaattcctagaatcaaacgctgtcaaataccttgaaggccacggattccgatttgaaatgaaaaaaaaagaaggcgtcaaacgccttgaagaattattaccagctgcctcttcaggcaaaaacataaaacgaaccctagcagcaatgccagaagaagaaactaccgaagcaaacgcaggccaattcctgtcgttcgcctcactattcctaccaaaactcgtggttggcgaaaaagcctgtctggaaaaagtacaacgacaaatccaagtacatgcagaacaaggattaatacaatatcccacgtcatggcaatcagtgggacacatgatggtcatcttcagactcatgcgcacaaatttcctaataaagtttctcctaatccaccaaggcatgcatatggtcgcaggacatgacgccaacgacgccgtcatagcgaactccgtcgcccaagcccgtttctcaggcctcctaatagtaaaaaccgtactcgaccacatcctccagaaaactgaacacggtgttagactccatccactagcccgcacggcaaaggtaaaaaatgaggtatcctcctttaaagccgcacttgcatccctagcacaacacggcgagtatgcaccattcgcccgacttctcaacctctcaggggtaaacaatctagaacacgggctcttcccacaactatcagcaatcgctctaggcgtcgcaacagcacacggaagcacacttgcaggagtaaacgtaggagaacaatatcaacaactccgagaagcagcaacggaggcagaaaaacaactccagaagtatgcagaatcgagagaactcgaccacttaggacttgacgatcaagaaaaaaaaattctcaaagattttcatcaaaaaaagaacgaaatctcatttcagcaaacaactgcaatggtcactctacgcaaggaacgactggcaaaattaacagaggcaataacatctacaagcatcctaaaaacaggacgaagatatgatgatgataacgatataccattccccggccccataaacgacaacgaaaattcagggcaaaacgacgacgatccaaccgacagccaagatactacaatacccgacgtaataatcgaccccaatgatggcggctataacaattacagtgattatgcaaacgacgcggcctcagcccccgacgatctcgtcctgttcgacttagaagatgaagacgacgccgataatccagcacaaaacacacctgagaaaaacgaccgtccagccaccaccaaactccgaaacggacaagatcaagatggcaatcaaggcgagacagcctcaccaagggttgcacctaatcaatatcgcgataaacccatgccccaagtccaggatcgatccgaaaatcatgaccaaaccctccaaacccaatcacgcgttctcaccccaataagcgaagaagctgatccttcagatcacaacgacggagacaacgaaagtatccccccactagaatcagacgatgaaggttcaactgacacaacagctgcagaaacaaaacctgccacagcgccacccgctcctgtctataggtcaatatcagtcgacgactcagtacccagcgaaaacataccagctcaatctaatcaaacaaataacgaagataacgtacgaaacaacgcccaatccgaacaaagcatcgcagaaatgtaccaacatatactcaagactcaaggcccattcgacgccattctttactaccacatgatgaaagaagaaccaatcatattctcaacatcagacggcaaagagtacacatacccagacagcctagaagacgaatatcccccctggctcagtgaaaaagaagccatgaacgaagacaaccgctttataacaatggacggacaacaattttactggccagtaatgaaccatagaaacaaatttatggccatacttcaacatcaccgataa

SEQ ID NO: 195 - RNA, 2217 nt, synthetic construct
  misc_feature 1..2217: note = SUDV NP, Gulu, Uganda 2000,optimized mRNA Sequence
  source 1..2217: mol_type = other RNA
atggacaaacgcgttagaggttcatgggccctaggtggacaatcagaagtcgacttagactaccacaaaatattaaccgcaggactctccgtacaacaagggatagtaagacaacgagtcataccagtctatgtcgtatccgacctagaaggtatatgccaacacataattcaagcttttgaagccggagtagatttccaagacaacgcagattctttcctactccttctttgcctgcaccacgcttatcaaggagaccatagactcttcctaaaatctgatgccgtacaatacctagaaggccatggttttcggttcgaagtgcgcgaaaaagaaaacgtccacagactcgatgaactcttaccaaacgtaaccggcggcaagaacttacgacgtacattagcagccatgccagaggaagaaacaacagaagcaaacgccggacaattcctaagcttcgcatcattattcctcccaaaactagttgtcggagaaaaagcctgccttgaaaaagtccaacgtcagatacaggtgcatgcagaacagggacttatacaatatccaacctcttggcaatccgtcggacacatgatggtcatattccggttaatgcgaacaaattttttaatcaaatttctcctaattcatcaaggaatgcacatggtggcaggacacgacgccaacgacaccgtcatatccaactcagtcgcgcaagcacgcttttcaggtcttctcatcgtcaaaaccgtactagatcacatcttacaaaaaacagatctcggggtaagactacaccccctcgcacgcaccgccaaagtaaaaaatgaagtatcctcttttaaagccgcactaggctcactagccaaacacggggaatatgctcccttcgcacgtctattaaacctatccggagtaaacaacctagaacacggcttatatccccaactctcagcgatagcactgggtgtcgcaacagcacacggatccactctagcaggagtaaacgtcggcgaacaataccaacaattacgagaagccgccacagaggccgaaaaacaactacaacaatacgccgaaaccagagaactagacaaccttggactcgatgaacaagaaaagaagatcttgatgtcatttcaccaaaaaaaaaatgaaatctcatttcaacaaacgaacgccatggtcaccttaaggaaggaacgacttgcaaaattaaccgaagcgataacaactgctagtaaaattaaagtaggcgaccgctaccccgacgacaacgatataccattccccggacccatatatgacgaaacacacccaaacccttctgacgacaacccagacgattcgcgagacacaaccataccaggaggtgtagtagatccgtacgacgacgagtcaaacaactacccggactatgaagactcagcagagggaaccaccggagacctcgacttattcaacttggacgatgacgatgatgattcacaacctggacccccagatagaggtcaatcgaaagaacgtgcggcaagaactcacggactccaagaccccacactagacggcgctaaaaaagtcccagaattaacacccggcagccaccaacccggcaaccttcacataacaaaaccaggatcaaacacaaaccaacctcaaggcaacatgtcaagcacactccaatcaatgacaccgattcaagaagaaagcgaacccgacgaccaaaaagacgacgacgacgaaagtctaacatcactagactcggaaggagacgaagacgtcgagtcagtctcaggcgaaaacaacccaacagtagccccgccagcacccgtatacaaagacacgggggttgacactaaccaacaaaacggaccatcaaacgcagtagacggccagggatcagaatccgaagcactacccatcaaccccgaaaaaggctcagcattagaagaaacttactatcatcttctgaaaacacaaggccccttcgaagcaataaactactatcacctaatgtctgacgaacccatagcatttagcacagaaagcggcaaagaatatatattcccagactcactagaagaagcatatcccccatggttatcagaaaaagaagctctagaaaaagaaaaccgctacctagtcatagatggacaacaattcctctggccagtaatgtcattacaagacaagttcctagccgtcctccaacacgattaa

SEQ ID NO: 196 - RNA, 2220 nt, synthetic construct
  misc_feature 1..2220: note = TAFV NP, Cote dIvoire 1994,optimized mRNA Sequence
  source 1..2220: mol_type = other RNA
atggaaagcagagcccacaaagcttggatgactcataccgcaagcggcttcgaaacagactaccacaaaatcctaactgcaggtctatcagttcaacaagggattgtccgccaaagagtcatccaagtacaccaagtaactaatctagaagaaatctgtcaactaataatccaagcttttgaagccggagtagactttcaagaaagtgcggacagcttcctcttaatgctatgtctacaccatgcataccaaggcgattacaaacagttcctcgaatcaaacgcagttaaatatctcgaaggtcatggatttcgtttcgaggtaaggaaaaaagaaggagtcaaacggctagaagaactccttccagccgccagctctggaaaatccatccgaagaacattggctgcaatgccagaagaggaaaccacagaagcaaacgcaggacaattcttaagctttgcatccttattcctccccaaactcgtagtaggcgaaaaggcatgcctcgaaaaagtgcaaaggcaaatacaagtacactcagaacaaggcctaatccaatacccaactgcatggcaatcagtaggccatatgatggttatcttcagactaatgcgcaccaacttcctcattaaatttctactaatacaccaaggcatgcatatggtcgcagggcacgacgctaacgacgcagttatcgccaactccgtcgcccaagccagattttctggcctattaatagtaaaaaccgttctagaccacatactccaaaaaaccgagcatggcgtgcgtctacacccacttgcacgtacggcaaaagtaaaaaacgaagtcaattcgttcaaagcagcactatcctctctcgcccaacatggagagtatgcgccatttgcccgactcctcaatctatccggagtaaataaccttgaacatggcctattcccccaactatccgccatcgcactaggcgtagctaccgcacacggctcaacattagccggagtcaacgttggagaacaatatcaacaactcagagaggcagccactgaagcagaaaaacaactccaaaaatatgccgaatcccgagaactcgatcacctaggcttagacgaccaagaaaaaaaaatcctcaaagacttccaccaaaaaaaaaacgaaatatcctttcaacagaccacagccatggttactcttaggaaagaacgattggccaaactcacagaagctataacaagcacttcactactcaaaaccggaaaacagtacgatgatgacaacgatataccattcccaggcccaataaacgacaacgaaaacagcgaacaacaagacgatgaccccaccgacagtcaagacacaacaatacctgatattatcgtagacccagacgatggacgatacaacaactatggagactacccatcagaaaccgccaacgcgcccgaagaccttgtcctattcgacctagaagatggagatgaagacgatcaccgaccctcatcaagcagcgaaaacaacaacaagcactcattaacaggaaccgacagcaacaaaaccagcaattggaatcgtaatcctacaaacatgccaaagaaagacagtacccagaacaacgataacccagcccaaagagcccaagaatatgccagagacaatatccaagatacaccaaccccacacagagccctaaccccgatatcagaagaaactggctccaacggacacaacgaggatgacatagactctatccctccactagaatcagatgaagaaaacaacaccgaaaccacaatcactaccacaaaaaacacaaccgctccacctgctccggtatacagatcaaacagcgaaaaggaacccctaccccaagaaaaaagccaaaaacaaccaaaccaagtgtcaggaagcgaaaatacagacaataaaccacactcagaacaatccgttgaagaaatgtaccgacacattctacagacccagggccccttcgacgcaatcctctactactacatgatgacagaagaacccatagtgttctcgacaagtgatggaaaagaatacgtctaccctgactcactagaaggcgaacaccctccatggttgagcgaaaaagaagccctcaacgaagacaaccgttttattacaatggatgatcaacaattctattggcccgtaatgaaccaccgaaacaaattcatggcaatcctccaacaccacaaataa

SEQ ID NO: 197 - RNA, 2031 nt, synthetic construct
  misc_feature 1..2031: note = EBOV GP, Mayinga, Zaire 1976, optimized mRNA Sequence
  source 1..2031: mol_type = other RNA
atgggagtaacagggatattacaacttcccagagacagatttaaaagaacaagcttcttcttatgggtaataatactattccaacgtacattttcaatcccactaggagtaatacacaacagtacattacaagtcagcgatgtagataaactggtatgtcgcgataaactatcatcaacaaaccaattaagaagcgtaggactaaatttagaaggaaacggagtagcaacagacgttccttcagctactaaaagatggggcttcagatctggagtaccaccaaaagtggttaattatgaagctggagaatgggcagaaaactgctataacctagaaataaaaaaacccgatggatcagaatgtctcccagccgctccagacggaataagaggatttccacgatgtagatatgtccacaaagtatcaggaactggaccatgcgctggtgattttgcctttcataaagaaggcgcttttttcttatatgatagactagcatcaacagttatttatagaggaactacatttgcagaaggagtagtcgcatttctaatattaccacaagccaaaaaagactttttctctagtcatcccctaagagaacccgtcaatgccactgaagacccatcaagcggatactattctactacaataagataccaagcaacaggatttgggacaaacgaaacagaatatttatttgaagttgataatttaacatacgttcaattggaatcaagatttacacctcaatttctgttacaactgaacgaaaccatatacacatcaggcaaaagaagtaacacgaccggaaaactaatctggaaagtaaatccagaaatagacacgactataggagagtgggcattttgggaaactaaaaaaaacttaacaagaaaaataagatccgaagaactctcattcacagtagtaagcaatggagcaaaaaacatatcaggtcaaagtccagcaaggacatcatcagaccccggaacaaacaccaccacagaagatcacaaaataatggcaagcgaaaattcatcagcaatggtccaagtccattcacaaggcagagaagcagctgtatcacatttaacaacattggcaacaatatcaacttcaccacaaagtctaactacaaaaccaggaccagacaatagtactcataatacaccagtttataaattagatatatcagaagcaacacaagtagaacaacatcaccgaagaacagataatgatagtacagcttcagacacaccctcggcaacaacagccgcaggaccacccaaagcggaaaatacaaacacaagtaaatccacagatttcctagatccagcaacaactacttcaccacaaaaccactctgaaacagcaggcaacaacaatacacaccatcaagacacgggagaagaaagtgcttcaagtggaaaattaggcctaataactaatactatcgctggagtcgcaggattaataactggtggaagacgaacacgccgagaagctatagtaaacgctcaaccaaaatgtaatcccaacttacactattggacaactcaagacgaaggagcagcaataggactagcttggataccatattttggaccagcagcagaaggaatctatatcgaaggccttatgcataatcaagatggcctcatatgtggattgcgccaattagccaatgaaacaacacaagccttacaattattcctacgtgcaacaacagaactacgaactttttcaatattaaatagaaaagcaattgatttcttactacaacgatggggtggaacctgtcacatattaggacctgattgctgtatagaaccacacgattggacaaaaaacataacagacaaaatagaccaaataatacacgattttgtggataaaacattacctgatcaaggagacaatgacaactggtggaccggatggagacaatggataccagcaggcatcggagtaacaggtgtaattatagctgtaatagctctattctgcatatgcaaattcgtattctga

SEQ ID NO: 198 - RNA, 2031 nt, synthetic construct
  misc_feature 1..2031: note = EBOV GP, Sierra Leone 2014, optimized mRNA Sequence
  source 1..2031: mol_type = other RNA
atgggtgtaacaggtatcttacaactaccaagagacagatttaaacgtacatcatttttcttatgggtcataatactatttcaacgaacttttagcataccacttggcgtaatccataactcaactcttcaagttagcgatgtagataaacttgtatgtcgcgataaattatcctcaacaaaccaattaagatcagtaggcctcaatttagaaggcaatggagtcgcaacagacgtaccctctgttacaaaacgttggggattcagaagtggggtcccgccaaaagtagtaaactatgaagcaggcgaatgggcagaaaattgttataatcttgaaataaaaaaaccagatggttcagagtgtcttccagcagcccccgacggcatacgtggattcccacgctgtagatacgtacacaaagtatcaggtacaggaccatgcgcaggggactttgcctttcataaagaaggagcatttttcttatatgatagattagcttcaaccgttatctacagaggaacaacctttgcagaaggagttgtagccttcttaatcttaccccaagcaaaaaaagatttcttttcttcacatcctctaagagaaccggttaacgccaccgaagacccatcttcaggctactatagtacaacaataagatatcaagcaacaggatttggaacaaacgaaacagaatacctattcgaagtagataatctaacatatgtccaactagaaagcagattcacaccacaatttttactacaactaaacgaaacaatctatgcaagtggaaaacgatcaaatacgacaggtaaacttatatggaaagtaaacccagaaatagatacaaccatcggcgaatgggcattttgggaaacaaaaaaaaacctaacacgaaaaatcagatctgaagaactctcattcactgcagtttcaaatggaccaaaaaatatatcaggtcaatcaccagcaagaacgtcatcagaccccgaaactaataccacaaacgaagatcataaaattatggctagtgaaaattcatcagctatggtccaagtccactcacaaggccgcaaagcagcagtatcacacctaactaccttagcaactataagcactagccctcaaccaccaacaacaaaaacaggcccagataattctacacacaacacaccagtatacaaacttgacatatcagaagcaacccaagtcggacaacatcacagaagagcagacaacgattcaacagcatctgatacacctcccgcaaccactgcagctggcccactcaaagctgaaaatacaaacacttcaaaaagcgcagactcattagatctagccacaacaacaagtccccaaaactattcagaaacagccggaaataataatacacatcaccaagataccggagaagaatcagcatcatcaggcaaattaggactaataacaaatacaatcgcaggtgtagctggacttataacaggcggtagaagaaccagaagggaagtaatagtaaacgctcaaccaaaatgtaatccaaacctacattactggacaactcaagatgagggagcagctataggcttagcatggataccatactttggaccagcagcagaaggaatatatacagaaggactaatgcataatcaagacggcctaatatgcggtctacgacaactagcaaacgagacaactcaagcacttcaactattcttacgtgctacaactgaactaaggacattctcaatactaaatagaaaagcaatagactttttactacaaagatggggaggtacatgtcatatactaggaccagattgctgtattgaacctcatgattggactaaaaacattacagataaaatagatcaaatcatccacgatttcgtagacaaaacattaccagatcaaggagacaacgacaattggtggacaggttggcgacaatggataccagcaggaataggtgtaacaggagttatcatagctgtcatcgcactattttgcatatgtaaattcgtattttaa

SEQ ID NO: 199 - RNA, 2046 nt, synthetic construct
  misc_feature 1..2046: note = MARV GP, Angola 2005, optimized mRNA Sequence
  source 1..2046: mol_type = other RNA
atgaaaacaacctgcttactaatctctttaatactcatacaaggagtcaaaaccctaccaatacttgaaatcgcatcaaacatccaaccacaaaacgttgactcagtatgcagtggcacattacaaaaaacagaagacgtacatctaatgggatttacactcagcggacaaaaagtcgcagactctccactagaagcaagtaaaagatgggcatttagagctggagttccccccaaaaacgtcgaatatacagaaggagaagaagctaaaacttgttataacatatctgtaacagacccatctggaaaatccctactcttagatccaccaacaaacataagagactatccaaaatgcaaaaccatacaccacatccaaggacaaaacccacacgcacaaggaatcgcattacacctttggggagcattctttctttatgatcggatcgcttcaactactatgtatcgcggaaaagtcttcacagaaggtaacatagcagcgatgatagtcaacaaaacagtacacaaaatgatattcagtagacaaggacaaggataccgacatatgaacctcacatcaacaaataagtactggacttcatccaacggaacacaaactaacgacacaggatgttttggtacattacaagaatataattcaacaaaaaaccaaacttgtgcaccatctaaaaaaccattaccattaccaactgcccaccccgaagtaaaactaacatctacatcaacagatgccacaaaactaaatactacagatccaaattcagacgatgaagacctcaccacttccggctcaggctccggagaacaagaaccatatacaacatctgatgcagccacaaaacaaggactatcttcaaccatgccaccaacaccatcacctcaaccatccacaccacaacaaggaggcaacaatacaaatcactcacaaggcgtagtcaccgaaccaggtaaaactaacacaactgctcaaccaagcatgcctccccacaacacaaccacaatatcgactaacaacaccagcaaacataatctatcaacaccaagcgtacccatccaaaatgcaacaaactataacacccaaagcacagccccagaaaatgaacaaacatcagcaccatcaaaaacaacattactccccacagaaaatcctacaacagcaaaaagcacgaatagtacaaaatccccaacaactaccgtaccaaacacaacaaacaaatattcaacttcgccgtctcccacaccaaactccactgcacaacacttagtatatttccgcagaaaacgtaacatcttatggagagaaggcgatatgttccctttcctagacggcttaataaatgcgcctatagactttgaccccgtcccaaacactaaaacaatattcgatgaaagctcatcctccggcgcatcagcagaagaagaccaacacgcaagtccaaacatatcattaaccttatcttatttccccaaagttaacgaaaacacagcacactcaggagaaaatgaaaacgactgtgacgctgaacttcgtatatggtccgtacaagaagacgatctcgcagctggcctatcatggataccgttttttggccccggaatagagggactctacaccgctggactcataaaaaaccaaaataacctagtctgcagactacgccgcctagcaaatcaaacagcaaaatcactcgaacttctcctcagagtcacaacagaagaacgcactttttcactaataaaccgacacgccatagacttcttactagcaagatggggaggaacttgtaaagtcctaggaccagattgttgtataggcattgaagacttatctagaaacatctccgaacaaatagaccaaataaaaaaagatgaacagaaagaaggaactggatggggactaggaggtaaatggtggacttccgactggggagttctcaccaacctaggaatcctactactcctatcaatagcagtcttaatagcactctcctgcatatgccgtatattcaccaaatacataggctaa

SEQ ID NO: 200 - RNA, 981 nt, synthetic construct
  misc_feature 1..981: note = EBOV VP40, Mayinga, Zaire 1976, optimized mRNA Sequence
  source 1..981: mol_type = other RNA
atgagaagagtaatactaccaacagcaccacccgaatacatggaagcaatataccccgtaagatcaaactcaacaatagcacgaggaggaaattcaaacaccggatttctaacaccagaatccgtaaacggagacacaccatctaacccactaagaccaatagccgacgacacaatagaccacgcatcacacaccccaggctcagtatcatcagcattcattctagaagcaatggtcaacgtaataagcggcccaaaagtactcatgaaacaaataccaatatggctacccctaggcgtcgccgatcaaaaaacctactcttttgactcaacaactgcagcaataatgctcgcctcatacactataacccacttcggaaaagcaacaaacccattagtaagagtaaatagattaggacccggaatacctgaccatccactaagattattaagaataggaaaccaagcattcctacaagaattcgtattaccaccagtacaactaccacaatactttacctttgacctaacagcactaaaactcataacacaaccactaccagcagcaacatggacagacgacacaccaacaggctcaaacggagcactaagaccaggaatatcattccacccaaaactaagacccatactactaccaaacaaatcaggaaaaaaaggaaactctgcagatctaacatcaccagaaaaaatacaagcaataatgacctccctacaagacttcaaaatcgtacccatagacccaacaaaaaacataatgggaatagaagtaccagaaacactagtacacaaactaaccggaaaaaaagtcacctcaaaaaacggacaaccaataataccagtattactcccaaaatacataggacttgacccagtagcaccaggcgacttaacaatggtaatcacacaagactgtgacacatgtcactcaccagcatcactcccagcagtaatagaaaaataa

SEQ ID NO: 201 - RNA, 981 nt, synthetic construct
  misc_feature 1..981: note = EBOV VP40, Sierra Leone 2014,optimized mRNA Sequence
  source 1..981: mol_type = other RNA
atgagaagagtaatcctaccaacagccccacctgaatacatggaagcaatatacccagcaagatctaactcaacaatagcaagaggaggaaactcaaacaccggattcctaacacccgaatctgtaaacggagacacaccctcaaaccccctaagaccaatagcagacgataccatagatcatgcatcacacacaccaggctcagtctcatccgccttcatactagaagcaatggttaacgtaatatcaggaccaaaagtcctaatgaaacaaataccaatctggttaccactaggagtcgccgaccaaaaaacatactccttcgactcaacaacagcagcaataatgctcgcctcctataccataacacacttcggtaaagccacaaacccattagtaagagtaaacagactaggcccaggaatacccgaccacccactaagattactacgaataggaaaccaagcattcctacaagaattcgtactaccaccagtacaactaccacaatacttcacatttgacctaacagcactaaaactaataacacaacccctacccgctgcaacatggaccgacgatacaccaaccggatcaaacggcgcattaagacccggaatatccttccacccaaaacttagaccaatattactacccaacaaatcaggaaaaaaaggaaattccgcagacctaacatcacccgaaaaaatacaagcaataatgacatctctacaagacttcaaaatagtaccaatagacccaacaaaaaacataatgggaatagaagtaccagaaacactagtacacaaattaacaggaaaaaaagtaacatccaaaaacggacaaccaataataccagtcttactacctaaatatataggattagacccagtcgccccaggagacctaaccatggtaataacacaagactgtgacacctgccactcacccgcctcactaccagcagttgtcgaaaaataa

SEQ ID NO: 202 - RNA, 912 nt, synthetic construct
  misc_feature 1..912: note = MARV VP40, Angola 2005,optimized mRNA Sequence
  source 1..912: mol_type = other RNA
atggcctcatcctcaaactacaacacatacatgcaatacttaaacccaccaccatacgcagaccacggagcaaaccaactaataccagcagaccaactctcaaaccaacaaggaataacaccaaactacgtcggagacctaaacttagatgatcaattcaaaggaaacgtatgccacgcattcacactagaagcaataatagatatatcagcatataacgaaagaacagtaaaaggagtaccagcatggctaccattaggaataatgtcaaacttcgaatatccactagctcacacagtagcagcactcttaacaggatcctatacaataacacaattcacacacaacggacaaaaatttgtacgagtcaacagactaggaacaggaataccagcacaccccctacgaatgttaagagaaggcaaccaagcattcatacaaaacatggtaataccaagaaacttctcaacaaaccaatttacatacaacttaacaaacctagtattatcagtacaaaaactcccagacgacgcatggagaccctcaaaagataaattaataggaaacacaatgcacccagcagtatcagtccatcctaatttacccccaatagtactaccaacagtaaaaaaacaagcataccgccaacacaaaaacccaaacaacggaccattactcgcaatttcaggcatactacaccaactaagagtagaaaaagtccccgaaaaaacttcattattccgaatatcactaccagcagacatgttctcagtaaaagaaggaatgatgaaaaaaagaggagaaaactcaccagtagtatactttcaagcacccgaaaacttccctctaaacggattcaacaaccgacaagtagtactagcatacgcaaacccaacactatccgcagtataa

SEQ ID NO: 203 - RNA, 2220 nt, synthetic construct
  misc_feature 1..2220: note = EBOV NP, Zaire 1976, optimized mRNA Sequence
  source 1..2220: mol_type = other RNA
atggatagtcgaccccaaaaaatatggatggcaccatcactaacagaatcagacatggattatcacaaaatacttacagctggtttaagcgtccaacaaggcatagtcagacaaagagttatacctgtttatcaagtaaacaaccttgaagaaatttgccaacttataattcaagcattcgaagctggagttgactttcaagagagcgcagacagcttcttactcatgctatgcctccaccacgcataccaaggagattataaactattcttagaatcaggtgcagtaaaatacctagaaggacatggctttagattcgaagttaaaaaaagagacggagtaaaaagacttgaagaattattacctgcagtcagcagcggaaaaaacataaaaagaacacttgccgcaatgcccgaagaagaaaccacagaagcaaacgcaggacaatttttatctttcgcctctctttttctgccaaaactcgttgtaggagaaaaagcgtgtctagaaaaagtacaaagacaaatacaagtacacgccgaacagggacttatacaataccccacagcatggcagtcggtcgggcacatgatggtcattttcaggctaatgagaacaaattttcttataaagtttttattgatacatcaagggatgcatatggtagctggccatgatgcaaatgatgcagtaatttcaaactcagttgctcaagcaagatttagtggattattaatagtaaaaacagtattagatcacatactacaaaaaacagaacgaggggtacgacttcatccactagcgaggacagcaaaagtcaaaaatgaagtaaatagcttcaaagcagcactatcttctctagcaaaacacggagaatatgcaccgtttgcaagattactaaatctatcaggagttaataaccttgaacacggtttattcccccaactaagtgcaatagccctaggagttgcaacagcccacggatcaacattagcaggtgtaaatgtaggcgaacaataccaacaattaagagaagcagccaccgaggcagaaaaacaattacaacaatatgcagaaagccgagaacttgatcacttaggattagatgatcaagaaaaaaaaattttaatgaattttcaccaaaaaaaaaacgaaatatcatttcaacaaaccaatgctatggtcaccttacgaaaagaaagattagcaaaattaaccgaagcaattacagcagcctcacttcctaagacctcaggtcattatgatgacgacgacgacatcccatttccaggaccaataaatgatgatgacaatccaggacaccaagacgatgacccaacggattcccaagatacaacaataccggatgtagtagtcgatcccgatgacggaagttacggagaatatcaatcatactcagaaaatggaatgaatgcaccagacgatttagttctattcgacttagacgaagatgatgaagacactaaacccgtcccaaacagatcaactaaaggtggacaacagaaaaattcacaaaaaggacaacatattgaaggaagacaaacacaatcacgacccatacaaaacgttcctggaccacatagaacaatccatcatgcatcagcaccattaacagataacgatagaagaaatgaaccctcaggctcaacaagtccaagaatgttaacaccaatcaatgaagaagccgatcctttagacgacgcagatgatgaaacatcaagtttaccacctctagaatccgatgatgaagaacaagacagagatggaacttcaaaccgaactccaacagtcgcaccaccagcccccgtatatcgagaccacagcgaaaaaaaagaattgccacaagatgaacaacaagatcaagatcatacacaagaagcacgtaatcaagatagtgataacacccaaagtgaacatagctttgaagaaatgtaccgacacatactacgtagtcagggaccattcgacgcagtattatactatcatatgatgaaagacgaaccagtagtctttagcacatcagacggaaaagaatacacctatcctgattctctagaagaagaatatccaccttggttgacagaaaaagaagcaatgaacgaagaaaataggttcgtcacattagacggacaacaattttattggccagtcatgaatcacaaaaacaaatttatggctatattacaacaccatcaataa

SEQ ID NO: 204 - RNA, 2220 nt, synthetic construct
  misc_feature 1..2220: note = EBOV NP, Sierra Leone 2014, optimized mRNA Sequence
  source 1..2220: mol_type = other RNA
atggatagtcgaccacaaaaagtatggatgacacccagcctaacagaaagtgatatggactatcacaagatactaacagcaggattatcagtacaacaaggaatagtaagacaaagagtcataccagtttaccaagtaaataatctagaagaaatttgtcaactaataatacaagcatttgaagctggagtagacttccaagaatcagcagacagcttcttgctaatgctatgtttacatcacgcatatcaaggcgattacaaattatttctagaaagcggagcagtaaaatacttagaaggacacggctttagatttgaagttaaaaaatgtgatggagtcaaaagactagaagagctactacctgcggtttcaagcggcagaaatataaaacgaacattagcagctatgccagaagaagaaacaaccgaagcaaatgccggacaatttttatctttcgcttcacttttcctaccaaaactagtagtgggagaaaaagcatgcctagaaaaagtacaaagacaaatacaagttcatgcagaacaaggcttaatacaatacccaacagcttggcaatccgtaggccacatgatggttatattcagattaatgagaactaactttctgatcaaatttctattaatacaccaaggaatgcatatggtagccgggcatgacgcaaatgacgcagtcatttcgaacagcgtggcacaagctagatttagcggtttactaatcgtaaaaacagtattagatcacatactacaaaaaacggaaagaggagtacgacttcaccccctagcaagaacagccaaagtaaaaaacgaagtaaattcattcaaggcagccttatcatcactagcaaaacacggtgaatacgctccatttgcacgtctactaaatttatctggagttaataacctagaacatgggctttttccacaacttagtgcaatcgccctaggtgtggcaactgcccatggaagcacactagcgggagttaatgtaggtgaacaataccaacagcttagagaagcagccacagaagccgaaaaacaactacagcaatacgctgaaagtcgggaactagatcacttaggattagatgatcaagaaaaaaaaatactaatgaattttcatcaaaaaaaaaatgaaatatcattccaacaaaccaatgcaatggtaactctaaggaaagaaagactagccaaactcacggaagctataaccgcagcctcattacctaaaacatcaggtcactatgatgacgatgatgatatacctttcccaggacctataaatgatgatgataacccaggacaccaagacgacgacccaacagattcccaagacaccacaataccagatgtagtagtagacccagatgacggaggatacggtgaatatcagtcatactcagaaaacggaatgtccgcaccagacgatctagtactatttgaccttgatgaagacgatgaagatacaaaaccagtaccaaacagatccacaaaaggaggacaacaaaaaaactcacaaaaaggacaacacacagaaggaagacaaacgcaatcaaccccgacccaaaatgtaacaggaccaagacgaacaatacaccacgcatctgcacccctcacagataacgacagaagaaatgaaccctcaggctcaacaagtccacgaatgcttacacccataaatgaagaagcagacccacttgatgacgccgacgatgaaacatcatcattaccaccattagaaagtgatgacgaagaacaggaccgcgacgggacctcaaataggacaccaacagtagcccctccagcccctgtatatagagaccatagtgaaaaaaaagaactaccacaagatgaacagcaagaccaagatcatatccaagaagcacgaaatcaagattcagataatacacaaccagaacactctttcgaagaaatgtatagacatatcctaagatctcaaggaccgtttgacgcagttttatattatcatatgatgaaagacgaacccgtggtattctcaacaagcgatggaaaagaatatacatacccagattctttagaagaagaatatccaccatggctaactgaaaaagaagccatgaatgacgaaaatagatttgtaactttagatggacaacaattttactggccagtaatgaaccacagaaacaaatttatggcgatacttcaacatcatcaataa

SEQ ID NO: 205 - RNA, 2031 nt, synthetic construct
  misc_feature 1..2031: note = BDBV GP, Uganda 2007,optimized mRNA Sequence
  source 1..2031: mol_type = other RNA
atggtaacctccggcatattacaactaccaagagaacgctttagaaaaacatcgttttttgtctgggtcataatattattccacaaagtcttccccatacccttaggagtcgtacacaacaatacccttcaagtatctgacatagataaattagtatgtagagacaaactcagctcaacatcccaactaaaatcagtaggactaaatttagaaggaaacggtgtagcaacagatgtaccaacagcaaccaaaagatggggatttcgtgctggagtaccaccaaaagtagtcaattacgaagctggagaatgggctgaaaattgctacaacctagatatcaagaaagcagatggatccgaatgtctcccagaagcaccagaaggcgtaagaggatttccaagatgccgatatgtacacaaagtaagcggtacaggaccatgtcccgaaggatacgcattccataaagaaggcgcctttttcttgtacgatagactagcatccacaatcatatacagaagcacaacattcagcgaaggcgtcgtagcattcctaatactaccagaaacaaaaaaagacttcttccaatcaccaccactccacgaaccagcaaacatgaccacagacccaagctcatattatcacacagttacactcaactatgtcgcagataacttcggaaccaacatgactaacttcctattccaagtcgaccaccttacctacgtacaattagaaccaagattcactccccagttcctagtccaattaaacgaaacaatttacacaaatggaagacgttcgaatactacaggaaccctcatatggaaagtaaaccccaccgttgacactggtgtaggagaatgggccttctgggaaaataaaaaaaacttcacaaaaacattatcatcagaagaactatcagtaatctttgtaccccgcgcacaagatccaggatccaatcaaaaaacaaaagtcactccaacatccttcgctaataatcaaacttcaaaaaaccacgaggatctcgtcccagaagatcctgcctctgtagtacaagtacgcgatctacaaagagaaaacacagtaccaactccacccccagacacagtacccacaaccctaataccagacacaatggaagaacaaactacaagccattacgaaccacccaatatatcacgcaaccaccaagagcgaaacaacacagcacatcccgaaacattagctaataatccacccgataatacaacaccttcaacaccaccacaagacggagaaagaacatcctcacatactacaccatcaccacgaccagttccaacaagcacaattcatcccaccacaagagaaacacacatcccaacaactatgacaacatcacatgacacagactcaaatagaccaaacccaatcgacatatccgaatcaacagaaccaggaccattaaccaacacaacaagaggagccgcaaacctcctgaccggatcccggagaacaagacgagaaataactttaagaacacaagcaaaatgcaacccaaacttacactattggaccacccaagatgaaggagcagccataggcctagcgtggataccttatttcggtcctgcagccgaaggcatatacactgaaggaatcatgcacaaccaaaatggactaatatgtggtctaagacaactagctaacgaaacaacacaagctctgcaactatttcttagagcaactacagaactaagaacattctctatactaaacagaaaagcaatagacttcctacttcaaagatggggaggaacttgccacatattaggacccgactgctgcatagaaccacacgactggacaaaaaatataacagacaagatagaccaaataatccacgattttatagataaaccactaccagaccaaacagacaacgataattggtggacaggttggagacaatgggttccagcaggaataggaataacaggtgtaataatcgccgtaatagcattactctgcatatgtaaatttttactttaa

SEQ ID NO: 206 - RNA, 2031 nt, synthetic construct
  misc_feature 1..2031: note = SUDV GP, Gulu, Uganda 2007, optimized mRNA Sequence
  source 1..2031: mol_type = other RNA
atgggcggtctatcactccttcaattacctagagacaaatttcgtaaatcatccttcttcgtttgggttataatactttttcaaaaggcattttcaatgccattaggtgttgtaacgaactcaacattagaagtcacagaaatcgatcaattagtttgcaaagatcatttagcctcaacagaccaacttaaatctgtcggactaaatttagaaggctcaggcgtatcaacagatataccttcagcaacaaaacgatggggattcagatcaggagttccaccaaaagtagtcagctacgaagcaggggaatgggccgaaaactgctataacctcgaaataaaaaaaccagatggatctgaatgtttaccacccccacctgacggagtaagaggattcccacgatgcagatatgtacataaagctcaaggaaccggaccatgccctggagactacgcatttcacaaagacggagcattctttctatacgatagactagcatccacagtaatatatagaggcgttaattttgccgaaggagtaatagcatttctcatattagccaaaccaaaagaaacgtttttacaatcaccacctattagagaagctgtaaattatacagaaaatacaagctcatattatgcaacatcatatttagagtacgaaatcgaaaatttcggcgcacaacactcaaccacattatttaaaattgataataatacattcgtaagactagatcgacctcacaccccccaattcctctttcaacttaatgatacaatccacctacatcaacaattaagtaatacaactggaagattgatctggacacttgacgctaacataaacgcagacataggcgaatgggccttttgggaaaataaaaaaaatttaagcgaacaattacgcggagaagaattatcattcgaagcattgtctcttaacgaaacagaagacgacgatgcagccagctcaagaataacaaaaggtagaatatcagatcgcgccacaagaaaatattctgatttagtacctaaaaattctccaggaatggttcccttacacataccagaaggtgaaactacactcccatctcaaaattcaaccgaagggagacgcgtaggagtaaacacacaagaaaccataaccgaaacagcagcaacaatcataggaacaaacggcaatcacatgcaaatatcaacaataggcataagaccatcatcctctcaaatcccatcatcatcccccacaaccgccccatcaccagaagctcaaacaccaactacacatacctctggaccaagtgtaatggctacagaggaaccaacaacacctccgggcagctcaccaggacctacaacagaagctccaactctaacaacaccagaaaacataaccacagcagtcaaaacagtactaccccaagaatcaacaagcaatggtctaataacatccacagtaacaggcatccttggaagcttaggtctaagaaaacgatctagaagacaaacaaacacaaaagcaacaggtaaatgtaatccgaatttacactattggacagcacaagaacaacacaatgcggcaggaattgcttggatcccatatttcggtcctggcgcagaaggtatatatactgaaggactaatgcataaccaaaatgcattagtatgcggactacgtcaactagctaatgaaacaacacaagccttacaactttttcttagagcaacaacagaattaagaacatatactatactaaacagaaaagctatcgattttttactcagacgatggggaggtacatgtagaatattaggacccgactgctgtatagaaccacatgactggacaaaaaacataacagataaaatcaaccaaataatccatgatttcatagacaatccacttcctaatcaggacaacgatgacaactggtggaccggatggcgacaatggatcccagcaggaataggaataacaggaattatcatcgcaattattgcattactatgtgtatgtaaactcctatgctaa

SEQ ID NO: 207 - RNA, 2031 nt, synthetic construct
  misc_feature 1..2031: note = TAFV GP, Cote dIvoire 1994, optimized mRNA Sequence
  source 1..2031: mol_type = other RNA
atgggcgcatctggaatactacaacttccacgagaacgtttcagaaaaacatctttttttgtatgggtaataatactattccacaaagtattctctatcccattaggcgtcgtacataacaacacactacaagtatccgacatagataaattcgtatgcagggacaagctatcctcaacatcacaactcaaatcagtaggactaaatctagaaggaaacggtgtagccacagacgtcccaaccgcaacaaaacgatggggatttagagcaggagtacctcctaaagtagtaaattgcgaagcaggagaatgggcagaaaactgctataacttagccataaaaaaagtagatggttccgaatgcttaccagaagccccagaaggagttcgcgatttcccacgatgtagatacgtacataaagtgtcaggcactggaccatgccccggtggtcttgccttccataaagaaggtgcatttttcttgtacgatagactagcttcaactataatctatcgtgggacaacattcgcagaaggagttatagcattcctaatattacctaaagcccgaaaagattttttccaatccccaccactccacgaaccagctaatatgacaactgacccatcaagctactaccacaccacaactataaactacgtagtcgacaacttcggaacaaatacaacagaatttctatttcaagtcgatcacctaacatacgttcaactagaagcaagattcacacctcaattcctagtcctcctaaacgaaactatttactcagataacagaagatccaatacaacaggaaaattaatatggaaaataaaccccaccgtagacacttcaatgggagaatgggcattctgggaaaacaaaaaaaacttcacaaaaaccctctcttctgaagaattatcattcgtacccgtgcctgaaacacaaaatcaagtactagatacaacagcaacagtatctccaccaatttctgcacataatcacgctgctgaagatcataaagaactagtatctgaagactctacccccgtagtacaaatgcaaaacataaaaggcaaagacacaatgcccactacagtaacaggagtaccaacaactacaccttcacccttcccaataaacgctcgaaatacagatcatacaaaatctttcattggactagaaggcccacaagaagatcattcaaccacacaaccagcaaaaacaacaagtcaacctacaaattctaccgagtctactactttaaatcctacctccgaaccatcatcccgaggcacaggaccttcttcaccaacagtccctaacaccaccgaatcccatgctgaattaggtaaaacaacaccaacaacattacccgaacaacacactgccgcctccgccatcccaagagccgtccacccagacgaactctcaggacccggattcctcacaaatacgattagaggcgtaacaaacctcctaacaggctcaagacgaaaaagacgcgatgttacacctaacacacaacctaaatgtaaccctaatttacactactggacagcactagacgaaggagcagcaataggtttagcttggataccttattttggtcccgcagcagaaggaatctacacagaaggtattatggaaaaccaaaatggactaatatgcggcttaagacaactagccaacgaaacaacccaagcattacaactattcttaagagcaacaaccgaactaagaacattctcaatactaaatagaaaagcaatcgatttcctactacaaagatggggtggaacttgccacatcttaggacctgactgctgcatagaaccacaagactggactaaaaacataacagataaaatagatcaaataatacacgatttcgtagacaacaacttacccaaccaaaatgacggatcaaattggtggactggctggaaacaatgggtgccagcaggaattggaatcaccggagtaataatcgcaataatcgcattactttgcatttgcaaattcatgctataa

SEQ ID NO: 208 - RNA, 981 nt, synthetic construct
  misc_feature 1..981: note = BDBV VP40, Uganda 2007, optimized mRNA Sequence
  source 1..981: mol_type = other RNA
atgagacgagccatattaccaacagcaccacccgaatacatagaagccgtctacccaatgagaacagtatcaacctcaataaactcaacagcatccggaccaaatttcccagctccagacgtaatgatgtcagacacaccatcaaactccttacgaccaatagcagacgacaacatcgatcacccctctcatacaccaacatctgtatcatcagcatttatacttgaagcaatggtaaacgtaatatcaggaccaaaagtacttatgaaacaaataccaatatggcttccactaggagtcgcagaccaaaaaacatactccttcgattcaacaacagcagccataatgttagcatcatacacaataacccacttcggaaaaacatcaaacccactcgtcagaataaacagattaggacccggaataccagaccaccccttacgactattaagaataggaaaccaagcattcctacaagaattcgtcttaccacccgtccaactcccacaatacttcacatttgacctaacagccctaaaactaataacccaacccctaccagcagcaacatggacagacgacacacccacaggacccacaggaatactaagacccggaatatcatttcacccaaaactaagaccaatactacttccaggaaaaacaggcaaaagaggctcaagctcagacctcacatcaccagacaaaatacaagcaataatgaacttcctacaagacctaaaattagtcccaatagatccagcaaaaaacataatgggaatagaagtaccagaactactagtacacagactaaccggaaaaaaaataaccactaaaaacggacaaccaataataccaatactactacccaaatacataggaatggacccaatatcacaaggagacctaaccatggttataacacaagactgtgacacatgccattcaccagcatccctaccaccagtatcagaaaaataa

SEQ ID NO: 209 - RNA, 981 nt, synthetic construct
  misc_feature 1..981: note = SUDV VP40, Gulu, Uganda 2000, optimized mRNA Sequence
  source 1..981: mol_type = other RNA
atgcgtagagtaacagtaccaacagcaccaccagcttacgcagatataggataccccatgtcaatgttaccaataaaatcctctagagcagtatcaggcatacaacaaaaacaagaagtattaccaggaatggacacaccatcaaattcaatgagaccagtcgcagacgataacatagaccacacatcacacacaccaaacggagtagcctcagcatttattttagaagcaacagtaaacgttatatccggacccaaagtcctaatgaaacaaattcccatatggttacccttaggaatagccgatcaaaaaacatactccttcgattcaacaactgctgcaataatgttagcatcctacacaataacacacttcggaaaagcaaataaccccctagtacgagtaaaccgattaggacaaggcatacccgaccatccattaagactactacgaatgggcaaccaagcattcctacaagaattcgtactaccaccagtacaactaccacaatacttcacattcgacctaacagcattaaaactagtaactcaaccattacctgctgctacctggacagacgaaacaccatcaaacctatcaggagcattacgaccaggactatcctttcacccaaaactcagaccagtactactaccaggaaaaacaggaaaaaaaggccacgtatccgacttaacagccccagacaaaatacaaacaatagtcaacctaatgcaagatttcaaaatagtaccaatagatccagcaaaatcaataatcggaatcgaagtaccagaactactagtacacaaactaacaggaaaaaaaatgtcacaaaaaaacggacaaccaataataccagtactactaccaaaatacataggactagatccaatctcaccaggagacctaactatggtaataacaccagactatgacgactgccactcacccgcatcatgctcatacctctcagaaaaataa

SEQ ID NO: 210 - RNA, 981 nt, synthetic construct
  misc_feature 1..981: note = TAFV VP40, Cote dIvoire 1994, optimized mRNA Sequence
  source 1..981: mol_type = other RNA
atgagacgcataatattaccaacagcacccccagaatatatggaagccgtctaccccatgagaaccatgaactcaggagcagacaacacagcatcaggaccaaattacacaactacaggtgttatgaccaacgacacaccatcaaactccttaagaccagtagccgacgacaacatagaccacccatctcacacaccaaactcagtagcctccgcattcatattagaagcaatggtaaacgtaatatccggacctaaagtattaatgaaacaaataccaatatggttaccacttggagtatcagaccaaaaaacctactcattcgactcaacaacagcagccataatgctcgcatcatacactatcactcacttcggaaaaacatcaaacccactagtaagaatcaatcgactcggtccaggcatacccgaccatcccctcagactactaagaataggaaaccaagcattcttacaagaattcgtactaccaccagtacaactaccccaatactttacattcgatttaacagctctaaaactaataacccaaccactaccagcagcaacatggacagacgaaaccccagcagtatcaacaggtacactccgaccaggaatctcattccacccaaaattaagacctatactactaccaggaagagccggaaaaaaaggatcaaactcagacctcacctccccagacaaaatccaagcaataatgaacttcctacaagacctaaaaatagtaccaatagaccctacaaaaaatataatgggaatagaagtcccagaactattagtacacagactaacaggaaaaaaaacaacaacaaaaaacggacaacccataataccaatactattacccaaatacataggactagaccccctatcacaaggtgacctaaccatggtaatcacacaagactgcgactcatgtcactcacccgcatcactcccacccgtaaacgaaaaataa

SEQ ID NO: 211 - RNA, 2088 nt, synthetic construct
  misc_feature 1..2088: note = MARV NP, Angola 2005, optimized mRNA Sequence
  source 1..2088: mol_type = other RNA
atggacttacattcactcttagaacttggaacaaaaccaacagctccacacgtgagaaacaaaaaagttatactattcgacaccaatcatcaagtctcaatatgtaaccaaatcatagacgcaataaattcaggaatcgacttaggagatctattagaaggcggcctcctaacactctgcgttgaacactactacaactctgacaaagacaaattcaatacatcacctatagcaaaatacctaagagatgccggatacgaatttgatgtcatcaaaaacgcagatgcaaccaggtttcttgacgtcataccaaacgaaccacattacagcccacttatactagccttaaaaaccctagaatctacagaatcacaaagaggacggattggactatttttatcattctgttcacttttcctaccaaaacttgtagtcggagacagagcatcaatagaaaaagccttaagacaagttaccgtacaccaagaacaaggaatcgtcacatatccaaaccattggctcacaaccggacatatgaaagtcatatttggaatactaagatcttccttcattctgaaatttgttttaatacaccaaggtgtcaacctcgtcaccggacacgacgcatatgactccatcataagcaattctgtcggacaaactcgtttctcaggattattaatagtaaaaactgtacttgaatttatactccaaaaaacagattccggcgtaacactacatccacttgtacgtacaagcaaagtcaaaaacgaagtagcctcatttaaacaagccctatcgaatctagcaagacatggagaatacgccccattcgctagagtactaaacttaagtggtataaataacctagagcatggcttatacccacaactttcggcaatcgcactaggagttgcaactgcacacggctcaactctagcaggagtaaatgtaggagaacaataccaacaactaagagaggcagcacacgatgccgaagtaaaattacaaagaagacatgagcatcaggaaatccaagcaatagctgaagacgacgaagaacgcaaaatactcgaacaattccacctccaaaaaacagaaataacacacagtcaaacactagccgtcctatcacaaaaaagagaaaaactagcaagattagcagcagaaatcgaaaataatatagtcgaggatcaaggtttcaaacaatcccaaaatagagttagccaatcattccttaacgaccccacacctgtagaagtaaccgtacaagcacgaccaataaaccgacccactgcactaccaccaccagtagactccaaaatagaacatgaaagtaccgaagattcaagctcatcttcatcgttcgtagatttaaacgaccccttcgcgctacttaacgaagacgaagacactttagatgactcagtaatgataccatcaacaactagcagagaatttcaaggaattcccgaacccccaagacaatcacaagatatcgacaactctcaaggaaaacaagaagatgaatctacaaacctaataaaaaaaccatttctcagataccaagaattaccaccagtccaagaagacgatgaatctgaatatactacagactcccaagaaagcatagaccaaccaggatcagataacgaacaaggtgtagacctaccacccccaccactatacgcccaagaaaaaagacaagacccaatacaacacccagctgtcagttcccaagatcctttcggctccataggagacgtaaacggtgatatactagaaccaatacgaagtccctcatcaccatcagcaccacaagaagacacccgtgcaagagaagcatatgaattatcaccagatttcacaaattacgaagacaatcaacaaaactggccacaaagagtagtaacaaaaaaaggtagaacatttctatacccaaacgacctactacaaactaaccctccagaatcattaatcacagcattagtagaggagtatcaaaatcctgtatcagcaaaagaactacaagccgactggcctgatatgtcattcgatgaacgaagacatgttgcaatgaatttataa

SEQ ID NO: 212 - RNA, 2220 nt, synthetic construct
  misc_feature 1..2220: note = BDBV NP, Uganda 2007, optimized mRNA Sequence
  source 1..2220: mol_type = other RNA
atggacccaagacccataaggacatggatgatgcacaacacaagtgaagttgaagcagactaccacaaaatactaaccgcaggacttagcgtacaacagggaatagtaagacaacgaataatacctgtatatcaaatatcaaacttagaggaagtctgccaacttattatccaagcattcgaagcaggagtcgactttcaagactccgccgatagcttcttactaatgctatgcctacatcacgcttaccaaggagactacaaacaattcctagagtctaacgcagtaaaatatctagaaggacacggattccgatttgaaatgaaaaaaaaagaaggcgttaaaagactagaagaattactaccagccgcatcatctggtaaaaacatcaagagaacattagctgcgatgccagaagaagaaacaacagaagcaaacgcaggtcaatttctatcctttgcttcactctttctaccaaaactagtagtaggagaaaaagcctgcctagaaaaagtacaaagacaaattcaagtacacgccgaacaaggcttaatacaatacccaacatcatggcaatcagtaggacatatgatggttattttccgcctcatgagaacaaacttcctaattaaattcttattaatacatcaaggaatgcatatggtagccggacacgacgcaaatgacgctgtaatagcgaacagcgtagctcaagcaagattcagtggattattaatagtaaaaaccgtactagaccatatcttacaaaaaacagaacatggagtaagactccacccactagctagaaccgcaaaagttaaaaacgaagtttcatctttcaaagccgcattagcatcactagcacaacatggagaatacgcaccattcgcccgacttctaaacttaagcggagtgaataacctagaacacggacttttcccacaactatcagccattgctctaggtgtagctacagcacatggatcaacactagcaggagtcaatgtcggcgaacaatatcaacaactcagagaagcagctacagaggcagagaaacaattacaaaaatatgccgaatcaagagagctggatcacctgggactagatgatcaagaaaaaaaaatactcaaagacttccaccaaaaaaagaacgaaatctcattccaacaaaccactgcaatggtaacattaagaaaagagcgtttagccaaactgacagaagccataactagcacatcaatcctaaaaactggacgaagatacgacgatgacaatgatataccttttccaggcccaattaacgacaacgaaaactcaggccaaaacgatgacgatcctacagattcacaagatacaacaatacccgacgtaattattgacccaaacgatggtggttataataactattcagactatgctaacgacgcagcctcagcaccagacgacctagttctttttgatttagaagatgaagacgatgcagacaatccagcacagaatacaccagaaaagaacgacagaccagctacaacaaaactaagaaacggacaagaccaagacgggaaccaaggcgaaacagcatccccaagagtcgcaccgaatcaataccgagataaaccaatgccacaagttcaagacagaagtgaaaatcacgaccaaaccttacaaactcaatcaagagtactaactccaatctcagaagaggccgacccaagcgatcataatgacggagataacgaatccataccaccactcgaaagtgatgacgaaggatcaacagacacaacagcagcagaaacaaaaccagcaacagcacctccagcaccagtttaccgaagcatatcagtagatgatagcgtaccaagcgaaaacatacctgctcaatcaaaccaaacaaacaacgaagacaacgtaagaaacaatgctcagagtgaacaaagcattgctgaaatgtaccaacacatattaaaaactcaaggaccattcgacgcaatattatattatcatatgatgaaagaagaaccaataatctttagtacaagtgacggaaaagaatacacgtatccggattcattagaagatgaatatcctccatggttatcagaaaaagaagcaatgaatgaagacaacagattcattacaatggacggccaacaattctattggccagtaatgaaccatcgaaataaattcatggctatattgcaacaccatagatga

SEQ ID NO: 213 - RNA, 2217 nt, synthetic construct
  misc_feature 1..2217: note = SUDV NP, Gulu, Uganda 2000,optimized mRNA Sequence
  source 1..2217: mol_type = other RNA
atggataaaagagtacgtggatcatgggcacttggtggacaatcagaagtagacttagactatcacaaaatactaacagcaggacttagcgtacaacaaggtatagtaagacaaagagtaattccagtatatgtagttagtgatcttgaaggaatttgtcaacacataatacaagcattcgaagcaggcgtagatttccaagataatgctgatagtttcctgctcttattatgtcttcaccacgcataccaaggagatcataggctatttctcaaatcagatgcagtacaatatttagaaggacatggttttagatttgaagtaagagaaaaagaaaacgtacatagattagacgaattattacctaatgtaactgggggcaaaaatttaagaagaacattagcagctatgccggaagaagaaacaacagaagccaacgctggtcaatttttatcattcgcatcactattccttcctaaattagtggtaggcgaaaaagcatgtttagaaaaagtccagagacaaattcaagttcatgcggaacaaggtttaatccaataccctactagttggcaatcagttggccatatgatggttatatttagactaatgagaaccaactttcttataaaattcctattaatacatcaaggaatgcatatggtagccggtcacgatgctaacgacacagtaatatccaattcagtagcccaagcacgattctcaggtttacttatagtaaaaacagtcttagaccacatattacaaaaaacagatttaggagttagactacaccctttagcacgaactgcaaaagtaaaaaacgaagtatcttcatttaaagcagcattaggaagccttgcaaaacacggagaatatgctccctttgcaagactcctaaatcttagcggagttaacaacctagagcatggattatatccacaattatcagcaatagcactgggtgtagcaacagcacacgggagtacactagcgggcgtaaacgtaggagaacaatatcaacaacttagagaagccgcaacagaagctgaaaaacaactacaacaatatgctgaaaccagagaattagataatctgggacttgacgaacaagaaaaaaaaatattaatgtcattccatcaaaaaaaaaacgaaataagtttccaacaaacaaacgcaatggttacattaaggaaagaacgactcgcgaaattaacagaagcaattacaaccgcaagcaagatcaaggtaggagacagatatccggacgacaatgatataccattcccaggccctatatatgacgaaacacatccaaacccaagtgatgataacccggacgatagcagggatacaactatacccggaggagtagtagatccctacgatgacgaaagcaataactaccctgattacgaagattctgcagaaggcactacgggagacttagatctatttaatctagatgatgacgatgacgacagccaaccaggaccacctgatagaggccaatccaaagaaagagctgctagaacacatggattacaggacccaacattagacggagctaaaaaagtaccagaattaacaccgggatcacaccaacctggaaatcttcatataacgaaaccaggcagcaacacaaaccaaccacaaggaaatatgtcttcaacattacaaagtatgacaccaattcaagaagaatcagaaccagatgatcaaaaagatgatgatgacgaaagtttaacatcgttggattcagaaggagatgaagacgtagaatctgtttccggagaaaacaatccaacagtagcaccacccgccccggtctataaagatacgggagttgatacaaaccaacaaaacggaccttcaaatgccgtcgacggacaaggttcagaatcagaagcactaccaattaacccagaaaaaggatcagctctcgaagaaacatattatcatctacttaaaacccaaggaccatttgaagcaattaactattaccacctaatgtccgacgaaccaatagcctttagtacagaaagcggaaaagaatatatatttcccgattcactagaagaagcatatccaccatggctttcagaaaaagaggcacttgagaaagaaaaccgatacttagtgatcgatggacaacaatttttatggccagtaatgtcgcttcaagacaaattcctagcggttctacaacacgattaa

SEQ ID NO: 214 - RNA, 2220 nt, synthetic construct
  misc_feature 1..2220: note = TAFV NP, Cote dIvoire 1994,optimized mRNA Sequence
  source 1..2220: mol_type = other RNA
atggagagcagagcacataaagcatggatgacccacacagcatccggattcgaaacagattatcataaaatactaacagcaggtctatcagtacaacaaggcatagtaagacaacgcgtaatacaagttcaccaagtaacaaacttagaagaaatctgtcaactaatcatacaagcattcgaagcaggcgtagatttccaagaatcggccgacagctttcttcttatgttatgtctacatcacgcataccaaggagattacaaacaattccttgaaagtaatgcggttaaatacctagaaggccatggatttagatttgaggtaagaaaaaaagaaggagtcaaaagattagaagaactcctacctgcagcatcctccggcaaatctataagaagaacactcgcagcaatgccagaagaagagaccactgaagcaaacgcaggccaattcctatcatttgcatcattattcctcccaaaactagtagttggtgaaaaagcatgcctagagaaagtacaaaggcaaatacaagtacactccgaacaaggattaatacaatatccaacagcctggcaatctgtaggtcacatgatggtcatctttcgactaatgagaacaaatttcttaatcaaattcctattaattcaccaaggaatgcacatggtagccggacatgatgcaaatgacgcagtaatagccaactcagtagctcaagccagattctcaggccttcttatagttaaaacagttttagaccacatcttacaaaaaacagaacatggcgtaagactacacccattagcaagaacagcaaaagtaaaaaacgaagttaattcttttaaagcagcattaagtagtctagcccaacacggtgaatatgcacccttcgcacgcctacttaatttatcaggagttaataatctcgaacacggtctattcccacaactatctgcaatcgcactcggagttgccaccgctcacggctcgacactcgcaggagtaaacgtcggtgaacaataccaacaacttagagaagcagcaacagaagctgaaaaacagctacaaaaatacgccgaatcgcgagaattagatcatttaggactcgacgaccaagaaaaaaaaatattaaaagattttcatcaaaaaaaaaacgaaatctcattccaacaaacaacagcaatggtaaccctacgcaaagaacgtctcgcaaaattaactgaagccataacttcaacatcgctactaaaaacaggtaaacaatacgacgacgataatgacatacccttcccagggcccataaatgataacgaaaactccgaacaacaagacgacgacccaacagactctcaagacacaactatcccagatatcatagtagatccagacgacggaagatataataactacggtgactaccctagtgaaacagcgaatgcacccgaagatctagtattatttgacctcgaagacggcgatgaagacgatcacagaccatcatcaagtagcgaaaacaataacaaacactccctaactggtaccgactcaaataaaacatcaaactggaaccgtaaccccacaaacatgcctaaaaaagactccacacaaaataacgacaaccctgcacaacgagcccaagaatacgcacgagataatatacaagacaccccaacaccacatcgcgcattaactcccatatccgaggaaactggatcaaatggccacaatgaagatgacattgactccataccaccacttgaatcagacgaggaaaacaacactgaaacaaccataacaacaacaaaaaacaccacagcaccacctgctccagtatacagaagtaattctgaaaaagaacccctacctcaagaaaaatctcaaaaacaacccaatcaagtatccggatcagaaaacactgacaataaaccacactcagaacaatcagttgaagaaatgtatcgacacatcctacaaacacaaggaccattcgatgcaatactatattactacatgatgacagaagaaccaatagtcttctcaacttcagatggaaaagaatatgtataccccgattcgctagaaggcgaacacccaccatggttatcagaaaaagaagcactaaacgaagacaaccgatttataaccatggacgatcaacaattctattggcccgtaatgaaccatagaaacaaatttatggcaatcctacaacaccataaataa

SEQ ID NO: 215 - RNA, 2031 nt, synthetic construct
  misc_feature 1..2031: note = EBOV GP, Mayinga, Zaire 1976, optimized mRNA Sequence
  source 1..2031: mol_type = other RNA
atgggagtcaccggaatccttcaactaccacgcgaccgcttcaaaagaacttcattcttcctctgggtcataatactattccaaaggacattctcaatacccttaggcgtcatccacaactctacactccaagtaagcgacgtcgacaaactagtgtgccgagacaaactatcatcaacaaaccaactacgcagcgtcggcctcaacctcgagggaaacggcgtagcaaccgacgtaccctccgcaacgaaacggtggggattccgttccggcgtcccccccaaagtcgtcaactatgaggccggagagtgggccgaaaactgctacaacctcgaaataaaaaaaccagatgggagcgaatgcctccccgccgcaccagacggaattcgcggctttccccgatgccgatacgtccacaaagtatccggcaccggaccctgtgctggtgacttcgccttccacaaggaaggagcattcttcctatacgaccgactcgcatccactgtcatttaccgaggcaccacgttcgccgaaggtgtcgtcgccttcctcatcctcccacaagcaaaaaaagacttcttcagcagccacccactacgcgaacccgtaaacgcaactgaagacccatcgtccggctactactcaacaaccatccggtaccaagcaacaggcttcggcactaacgaaaccgaatacctctttgaagtcgacaacctgacctatgtacaactagaatcccgcttcaccccccaatttttacttcaactcaacgaaacaatctacacctcaggaaaacgatccaacaccacaggcaagctaatctggaaagtaaaccccgaaatagacacaacaatcggtgaatgggccttctgggaaacaaaaaagaacctaacccgaaaaatccgttctgaagaactgtccttcaccgtagttagcaacggagccaaaaacatctcgggccaatcccctgctcgcacctcatccgaccctggcaccaacacgacaaccgaagaccacaaaataatggcatcagaaaattcctccgcaatggttcaggtccactcccaaggccgcgaagccgcagtctcccacctcaccaccctcgccaccatctccacctccccccaatcactaaccacaaaacctggaccagacaactcaacacataacacccccgtctacaaactagacatatccgaagcaacccaagtcgagcaacaccaccgacgtacagataacgattcaaccgcctccgacacccccagcgccaccaccgccgcaggcccaccaaaagcggaaaacaccaacacatctaaaagcacggacttcctagaccccgccacgacgacctccccccaaaaccactccgaaaccgcaggcaacaacaacacccaccaccaagacaccggtgaagaatccgccagctcaggtaaactaggcctcataactaacacaatagccggcgtagcaggactaatcaccggcggacgacgcacacgccgcgaggcaatagtcaacgcgcaacccaaatgcaacccgaacctacactactggacaacacaagacgaaggagcagccatcggcctagcctggataccgtacttcggcccagccgccgaaggcatatacatcgaaggcctcatgcacaaccaagacggtcttatctgcggtcttagacagctcgcaaacgaaaccacccaagccctgcaactcttcctaagagctactaccgaactacgcacattctccatcctcaaccgcaaagcgatcgacttcctcctccaacgctggggcggcacatgtcacatcctcggccccgactgctgtatagaaccgcacgactggaccaagaacataacagacaaaattgatcaaatcatccacgacttcgtcgacaagaccctccctgatcaaggcgacaacgacaactggtggaccggctggcgtcaatggataccagccggaatcggcgtgaccggcgtcattattgcggtaattgcactattctgcatctgcaagttcgtcttctga

SEQ ID NO: 216 - RNA, 2031 nt, synthetic construct
  misc_feature 1..2031: note = EBOV GP, Sierra Leone 2014, optimized mRNA Sequence
  source 1..2031: mol_type = other RNA
atgggcgtcactggcatcctacaactcccccgcgaccgctttaaacgaacaagcttctttttgtgggttatcatcctcttccaacgcacattctccatcccactgggtgtcatccacaactccaccctacaagtctcagacgtcgacaaactagtctgccgcgacaaactctcatccactaaccaactacgatccgtagggctaaacctagaaggcaatggcgtcgccaccgatgtcccctccgtaacaaaacgatggggcttccgcagcggagtgccacccaaagtcgtcaactacgaagctggagaatgggcagaaaactgctacaacctcgagatcaaaaaaccagacggttcagaatgccttccagccgcaccggacggaataagaggctttccacgatgccgatatgtccataaagtctccggaaccggcccatgtgctggagacttcgcctttcacaaagaaggcgccttcttcttatacgaccgcctcgcctcgacagtcatctaccgcggcacaaccttcgcggaaggcgtcgtcgcattcctaatactcccccaagccaaaaaagacttcttctcctcacaccccctccgcgaaccggtaaacgccaccgaagaccccagttccggttactactccaccaccatccgctaccaagcgaccggattcggcacaaacgaaacagaatatctcttcgaagtcgacaacctcacctacgttcaactcgaatcgcgtttcacaccgcagtttctactacaactcaacgaaaccatctacgccagcggcaaaagatccaacaccacaggtaaactaatctggaaagtaaaccccgaaatagacaccaccataggcgaatgggccttttgggaaacaaagaaaaacctcacccgcaaaatacgatctgaggaactaagcttcaccgccgtctcaaacggcccaaaaaacatatccggccaatcgcccgctcggacctcctccgaccccgagactaacaccaccaacgaagaccacaaaataatggcaagcgagaactcatcagctatggtccaagtccattctcaaggacgcaaagccgcagtcagccacctcacaactctcgcaacaatctccaccagcccccagccccccaccacaaaaacaggcccagacaactcaactcacaacacaccagtctacaaactcgacatctccgaagccacccaagtcggacaacaccaccgccgcgccgacaacgactccaccgcctccgacacgccgcctgccaccaccgccgccggccccctcaaagccgaaaacaccaacacatccaaatccgccgacagcctcgaccttgccaccacaacctccccccaaaactactccgaaaccgccggcaacaacaacacccaccatcaggacaccggcgaagaatcagcctccagcggcaaactcggactcataaccaacacgatcgctggtgtcgccggactcatcaccggcggacgacggacccgccgcgaagtcatcgtcaacgcacaacctaaatgcaacccaaacctacattactggaccacacaagacgaaggtgcagccataggactcgcctggatcccatacttcggccccgccgccgaaggcatctacacagaaggcctgatgcacaaccaagacggactaatctgcggcctacgacaactcgctaacgaaaccacacaagctctccaactatttctacgcgcaaccacagaactacgaacgttctccatactcaaccgaaaagctatcgacttcctactacaacgctggggtggcacctgccacatactcgggcccgactgctgcatagaacctcacgactggacaaaaaacataaccgataaaatcgatcaaatcatacacgacttcgttgacaaaacattgccagaccagggtgacaacgataactggtggaccgggtggcgtcaatggatcccagctggcataggcgtaacaggagtaatcattgcggtcattgccctcttctgcatatgcaaattcgtcttctga

SEQ ID NO: 217 - RNA, 2046 nt, synthetic construct
  misc_feature 1..2046: note = MARV GP, Angola 2005, optimized mRNA Sequence
  source 1..2046: mol_type = other RNA
atgaaaacaacatgccttctgatctccctcatactcatccaaggcgtcaagacgctccccattctcgaaatcgcatccaacattcagccccaaaacgttgactccgtatgctcaggaaccctgcaaaaaacagaagacgtacacctcatgggatttaccctctcaggccaaaaagtggccgactcccccctagaagcatccaaacgatgggccttccgcgccggcgtaccccccaaaaacgtagaatacaccgagggcgaagaagcaaaaacctgctacaacatatcagtcacagacccatccggaaagtcactccttctagacccccccaccaacattcgagactaccccaaatgcaaaacaatccaccacatccagggccaaaacccccacgcccaaggaatagccctccacctctggggagctttcttcctctacgaccgcatcgcatccaccaccatgtaccgaggcaaagtattcaccgaaggaaacatcgccgcaatgatcgtcaacaaaaccgtccacaaaatgatcttctcccgccaaggacaaggctatcgccacatgaacctcacctccacaaacaagtactggacttcctcaaatggcacgcaaaccaacgacaccggatgcttcggcaccctccaggaatacaactcaacaaaaaaccaaacatgcgccccctccaaaaaacccctaccgctccccacagctcaccccgaagtaaaactcacctctacttccacagatgcaactaaactcaacaccacagacccaaactcagacgacgaagacttaaccacaagcggatcaggctccggtgaacaagaaccttacaccaccagcgacgctgctacaaaacaaggcctctcttccaccatgccccccacaccctccccccaaccctccaccccccagcaaggcggaaacaacaccaaccactcccaaggcgtcgtcacggaacctggcaaaaccaacaccactgcccaaccttccatgccgccacacaacacaactacaatatccaccaacaacacctcaaaacacaacctttcaaccccctccgttcccatccaaaacgccactaactacaacacgcaatccaccgcacccgagaacgaacaaacctccgctccctctaaaacgaccctgctccccacagaaaacccaactacagccaaatcaacaaattccaccaaatctccgaccacaacagtccccaacacaaccaacaaatacagcaccagcccctcccccacaccaaactcaaccgcacaacacctagtatacttccgccgcaaacgaaacatcctatggcgagaaggagacatgttcccgttcctcgacggcctcatcaacgcgcccatcgacttcgatcccgtcccgaacaccaaaacaatcttcgacgaatcctcatcctcaggcgcttctgcggaagaagaccagcacgcctcaccaaacatatccctaaccctctcgtacttccccaaagtcaacgaaaacaccgcacactccggagaaaacgaaaacgactgcgacgcagaactccgcatctggagtgtacaagaagacgacctagccgcaggcctgtcatggatccctttcttcggccccggcatcgaaggactctacaccgcgggcctcatcaaaaatcagaacaacctcgtctgccggcttaggcgcctggccaaccaaaccgccaaatcactagaactcctgctccgagtcaccaccgaagagagaaccttcagccttattaaccgacatgctatagacttcctcctagcgagatgggggggcacatgtaaagtcctgggtcccgattgctgcataggcatcgaagacctatcccgaaacatatcagaacagatcgaccagatcaaaaaagatgaacaaaaagaaggcactggatggggcctaggcggtaaatggtggacatccgactggggagttctaaccaacctcggcatacttctcctcctatcaatcgccgttctcatcgcgctttcatgcatctgccgcatattcaccaaatacatcggttag

SEQ ID NO: 218 - RNA, 981 nt, synthetic construct
  misc_feature 1..981: note = EBOV VP40, Mayinga, Zaire 1976, optimized mRNA Sequence
  source 1..981: mol_type = other RNA
atgcgccgagtcattctacccaccgcccctcccgaatacatggaagccatataccccgtccgatccaactccacaatcgcccgcggcggaaactccaacacaggcttcctcacacccgaatcagtcaacggcgacacaccctccaacccactccgccccatcgccgacgacacaatcgaccacgcaagccacacccccggctccgtctcatcagccttcattctcgaagccatggtcaacgttatctcaggaccaaaagtcctcatgaaacaaatccccatctggctccccctcggcgtcgccgaccaaaaaacctactccttcgactccaccacagccgctatcatgctagcctcctacaccatcacacacttcggcaaagccaccaaccccctcgtacgcgtcaaccgcctaggcccaggcatccccgaccaccctttacgccttctccgcataggcaaccaggcattcctccaagaattcgtcctcccacccgtccaactcccccaatacttcaccttcgaccttaccgccctcaaactcatcacccaaccactacccgcagccacctggaccgacgacacacccaccggctccaacggcgccctacgccccggcatctctttccacccaaaactcagacctatcctactccccaacaaatctggaaaaaaaggcaactccgccgacctcacctcccccgaaaaaatccaagccatcatgacctccctccaagacttcaaaatagtacccatcgatccaacaaaaaacatcatgggcatcgaagtccccgaaaccctagtccacaaactcaccggcaaaaaagtcacctccaaaaacggccaacccatcatacccgtcctactccccaaatacatcggcctcgacccagtcgcccccggagacctaaccatggtaatcacacaagactgcgacacctgccactcacccgcctcactccccgccgtcatcgaaaaataa

SEQ ID NO: 219 - RNA, 981 nt, synthetic construct
  misc_feature 1..981: note = EBOV VP40, Sierra Leone 2014,optimized mRNA Sequence
  source 1..981: mol_type = other RNA
atgcgacgagtaatcctaccaaccgccccacccgaatacatggaagccatctaccccgctcgctcaaactccacaatagcccgcggcggcaactcaaacaccggattcctcacccccgaatccgtaaacggcgacaccccctccaacccactccgccccatcgccgacgacacaatcgaccacgcatcccacacccccggctccgtctcctccgccttcattctcgaagccatggtcaacgtcatatcaggccccaaagtcctcatgaaacaaatacccatctggctccctctcggagtcgcagaccaaaaaacctactccttcgactctaccaccgccgccatcatgctagcatcctacaccatcacccacttcggcaaagccaccaaccccctagtccgcgtcaaccgcctaggcccaggcatccccgatcaccccctccgcctcctccgcatcggcaatcaggcattcctacaagaattcgtcctacccccagtccaactcccccaatatttcaccttcgacctcaccgccctcaagctcatcacacaacctctaccagcagccacatggacagacgacacaccaaccggctccaacggcgccctccgcccaggcatctcctttcaccccaaactccgcccaatcctcctccccaacaaatccggcaaaaaaggcaattccgccgacctcacctcacccgaaaaaatccaagccatcatgacctccctacaagacttcaaaatcgtcccaatcgaccccaccaaaaacatcatgggcatcgaagtccccgaaactctagtccacaaactaaccggcaaaaaagtcacatccaaaaacggtcaacccatcatccccgtcctcctccccaaatacatcggcctcgaccccgtcgcacccggcgacctcacaatggtcatcacccaagactgcgacacatgccattccccagcctcactccccgccgtcgtcgaaaaatga

SEQ ID NO: 220 - RNA, 912 nt, synthetic construct
  misc_feature 1..912: note = MARV VP40, Angola 2005,optimized mRNA Sequence
  source 1..912: mol_type = other RNA
atggcctcctcctcaaactacaacacctacatgcaatacctaaacccgccaccatacgccgaccacggcgctaaccaactcatccccgccgaccaactatccaaccaacaaggcatcacccccaactacgtcggcgacctgaacctcgacgaccaattcaaaggcaacgtctgccacgccttcaccctcgaagccatcatcgatatctcagcctacaacgaacgcaccgtaaaaggcgtccccgcctggctccccctaggcatcatgtcaaacttcgaataccccctcgcccacaccgtagccgccctactcactggctcatacaccatcacacaattcacccacaacggccaaaaattcgtccgagtcaaccgcctcggaaccggcatccccgcccacccactccgaatgctccgcgaaggtaaccaagccttcatacaaaacatggtcatcccccgcaacttctccaccaaccaatttacatacaacctcaccaacctcgttctctccgtccaaaaactacccgacgacgcctggcgcccatccaaagacaagctcatcggcaacaccatgcaccccgccgtaagcgtccatcccaacctcccccccatcgtactaccaaccgtcaaaaaacaagcctatcgccaacacaaaaaccccaacaacggacccctcctagccatatccggcatactacaccagctccgtgtcgaaaaagtccccgaaaaaacctccctcttccgcatctccctccccgcagacatgttctcagtcaaagaaggcatgatgaaaaaacgcggcgaaaactccccagtagtctacttccaagcccccgaaaacttccccctcaacggattcaacaaccgccaagtcgtcctcgcctacgccaaccccaccctctcagccgtctaa

SEQ ID NO: 221 - RNA, 2220 nt, synthetic construct
  misc_feature 1..2220: note = EBOV NP, Zaire 1976, optimized mRNA Sequence
  source 1..2220: mol_type = other RNA
atggacagtcggccccaaaagatctggatggcccccagcttaaccgaaagcgacatggactaccacaaaattctgaccgccggcctctcagtccaacaaggaatcgtacgccaacgcgtaatacccgtataccaagtcaacaacctcgaggaaatctgccaactcattatacaggcattcgaagcaggcgtcgacttccaggaatccgccgactccttcctactcatgctatgcctccaccacgcctaccaaggcgactacaagctcttcctggaatccggcgctgtcaaatacctcgaaggtcacggcttccgcttcgaagtaaagaaacgcgacggcgtcaaaagactagaagaactcctccccgcagtctcctcaggaaaaaacatcaagcgaaccctggccgcaatgccagaagaggagaccaccgaagcgaacgcaggccaattcctgtccttcgcctcgctctttctgcccaaactagtcgtcggcgaaaaagcgtgcctcgaaaaggtacaacgacagatccaagtccacgcagaacaaggtctaatccaataccccacagcctggcaatccgtcggacacatgatggtcatcttccgactcatgcgcacaaacttcctgatcaaattcctcctcatccaccagggcatgcacatggtcgcaggccacgatgccaacgacgccgttatctcaaattccgtcgcccaagcccgcttctcaggcctcctcatcgtcaaaaccgtactagaccacatactacaaaaaaccgaacgcggggtccgcctccaccccctcgcacgcaccgcaaaggtcaagaacgaagtcaactccttcaaagccgcactctcctcactagcaaaacacggcgagtacgcaccattcgctcgactactaaacctcagcggagtcaacaatcttgaacacggcctcttcccccaactctcagcaatagcacttggagtagcgacagcccacggatcaacactcgcaggagtcaacgtcggtgagcaataccaacaattaagagaagccgctaccgaagcagagaaacagctccagcaatacgcagagtcccgagaactcgatcatctaggcctagacgaccaagagaaaaagatcttaatgaacttccaccagaagaaaaacgagatttccttccaacaaaccaacgccatggtaaccctccgcaaagaacggctcgccaaactgacggaggccatcaccgcagccagcctccccaaaaccagcggccactacgacgacgacgatgacatcccattcccaggacccatcaacgacgacgacaaccccggtcatcaagacgacgacccaacagacagccaagacacaacaatcccagacgtagtagttgaccctgacgacggaagctatggagaataccaatcctactcagaaaacggcatgaacgcccccgacgacctagtactatttgacctcgacgaagacgacgaagacacaaaaccagtacccaaccgctccacaaagggcggacaacaaaaaaactcacaaaagggccaacacatcgaagggagacaaactcagtcacgcccaatccaaaacgtcccaggaccacaccgaaccatccaccacgcatctgccccactgactgacaacgaccgacgcaacgaaccctcgggctcaaccagccctagaatgctcaccccaataaacgaagaagcagacccacttgacgacgccgacgacgagaccagctccctacccccattagaatccgacgacgaagagcaagacagggacggcactagcaacagaaccccaacagtcgccccaccagcaccagtctaccgcgaccactccgaaaaaaaagagctcccgcaagacgaacagcaagaccaagaccacacccaagaagctcgaaaccaagactccgacaatacacaatcagaacactcattcgaggaaatgtacaggcacatcttacgatcacaaggaccattcgacgcagtcctttactatcacatgatgaaagacgaaccagtcgtctttagcaccagcgacggcaaagaatacacataccccgactccctggaagaggagtaccccccgtggttaacagagaaagaagccatgaatgaagagaaccgatttgtcacgctcgacggtcaacaattctactggcccgttatgaaccacaaaaataaattcatggcgatcctacaacaccaccaataa

SEQ ID NO: 222 - RNA, 2220 nt, synthetic construct
  misc_feature 1..2220: note = EBOV NP, Sierra Leone 2014, optimized mRNA Sequence
  source 1..2220: mol_type = other RNA
atggactctagaccccaaaaagtatggatgaccccctccctcacagaaagcgacatggactaccacaagatcctaaccgccggcctatcagtacaacaaggaatagtccgccaacgcgtaatccccgtctaccaggttaacaacttagaagagatatgccaactcatcatccaagctttcgaagctggcgtagacttccaggaatccgccgacagcttcctcctgatgctgtgcctccaccacgcctaccaaggcgactacaagctattcctagaatccggagccgtaaaatacctagaaggccacggctttcgcttcgaagtaaaaaaatgcgacggcgtcaaacgactggaggaactcctcccagccgtaagctctggccgaaacataaaacgtacattagcagcaatgccagaagaagaaacaacggaagcaaacgccggccaattcctcagcttcgcctcactcttcctccccaagctcgtcgtcggagagaaagcctgcctcgaaaaggtacaaaggcaaatccaggtgcacgccgagcaaggcctgattcaataccccaccgcctggcaatccgtcggtcatatgatggtaatattccgcctcatgcgcaccaatttcctaatcaaattcctcctcatacaccagggcatgcacatggtcgcaggacacgacgcaaacgacgcagtcatctccaactccgtagcacaagcaaggttctcaggcctactcatcgtcaaaaccgttctagaccacatactacaaaaaacagaacgaggagtccggctccaccccctcgctagaacagcaaaagtcaagaacgaagttaactcattcaaggcagcgctatcctccctcgccaaacacggcgaatacgctccctttgcccgcctactaaacttgtccggtgtaaacaacctagaacacggtctattccctcaactctcagccatagccttaggcgtcgccaccgctcatggctccacactcgccggggtaaacgtcggcgaacaataccaacaactaagagaagccgcgacagaagcagaaaaacaactacagcaatatgcagaatcacgtgaactcgaccacctaggtctcgacgaccaagaaaaaaaaatcctcatgaattttcaccaaaaaaaaaacgaaatctcattccaacaaacaaatgccatggtaactctccgcaaagagcgactggccaaactaacagaagcaatcacggcagcctcactcccaaaaaccagcggacattatgacgacgatgacgacatacccttccccggccccataaatgacgacgacaaccccggccaccaggacgacgacccaaccgactcacaagatacaacaatccccgacgtagtcgtcgaccctgatgacggcggctacggagagtaccaatcatactccgaaaacggcatgtctgcccccgacgacctagtactctttgacctggacgaagacgacgaagacacaaaacccgtccccaaccggtccaccaaaggcgggcagcagaaaaacagccaaaaaggccaacacacggaaggacgccaaacccaatccacaccgacccaaaacgtcacaggccccagaagaacaatacaccatgcgtcagcacccctaaccgacaacgaccgacggaacgaaccatcaggctctacatccccccgcatgctcacccccatcaacgaggaagccgacccacttgacgacgcagatgacgaaacctctagcctcccgcccctagaatccgacgatgaggagcaagatcgcgacggaaccagcaaccgcacccctaccgtcgcaccgcccgcacccgtatacagggaccactcagaaaagaaagaactaccccaagacgaacaacaagaccaagaccacatccaagaagcccgcaatcaagactccgataatacgcagcctgaacactcattcgaagagatgtacagacacattctccgatcacagggacccttcgacgcagtcctctactaccacatgatgaaggacgaaccggtagtcttcagcactagcgacggaaaagaatacacataccctgactcactagaggaggaataccccccatggctcacagaaaaagaagcgatgaacgacgaaaaccgcttcgtcacactcgacggccagcaattctactggccagtcatgaaccaccgcaacaaattcatggctatcctccaacaccaccaatga

SEQ ID NO: 223 - RNA, 2031 nt, synthetic construct
  misc_feature 1..2031: note = BDBV GP, Uganda 2007,optimized mRNA Sequence
  source 1..2031: mol_type = other RNA
atggtgacctctggaatactacaactaccacgggaacgattccgcaaaacctctttcttcgtatgggtaatcatactattccacaaagtattccccatcccccttggagtcgtgcataataacacccttcaagtaagtgacatcgacaaacttgtctgccgagacaaactatcatccacatcacaacttaaatcagtcggcctcaatctcgaaggtaacggggtagccaccgacgtacccacagcaaccaaacgatggggcttccgagccggcgttccacccaaagtcgtcaactacgaggcaggagaatgggccgaaaactgctacaacctcgacatcaagaaggcagacggtagtgaatgcctacccgaagctcccgaaggcgtcagaggctttcccaggtgccgctacgttcataaagtatctggaaccggcccctgtccagaaggttacgcattccacaaagaaggcgccttcttcctatacgaccgcctcgcctccaccattatctaccgctccaccaccttcagcgagggagtagttgcctttctaatcctacccgaaacaaaaaaggactttttccaaagcccccccctccacgagcccgcaaacatgacaaccgatccctcatcctattaccacaccgttacactaaactacgtagccgacaacttcggcaccaacatgactaacttcctattccaagtcgaccacttaacgtacgttcaactcgagccccgattcaccccccaattcctagtacaactcaacgaaacaatctacactaacggccgacgctccaacaccacaggaaccctcatctggaaagtcaacccaacagtcgacaccggcgtcggagaatgggccttctgggagaataaaaaaaacttcaccaagaccctctccagcgaggaactctcggtcatcttcgtaccacgcgcacaagacccaggatctaaccaaaaaacaaaggtaacccccacctccttcgcaaacaaccaaacctccaaaaaccacgaagatctagtccctgaagaccccgcctctgtagtacaagtacgagatctccaacgcgaaaacaccgtccccactccccccccagacaccgttccaacaaccctaataccagacacaatggaggaacaaacaacatcccactacgagccccctaacatatcccgcaaccaccaggaacgtaacaacaccgcccatcccgaaactctcgccaacaaccccccagacaacacaaccccctcaacacccccccaagacggagaacggacatcctcccacaccaccccctccccccgcccagtacccacctcaaccatccaccccacaacccgcgaaacacacatccctacaaccatgaccacctcccacgacacagactccaaccgccccaaccctatcgacatctccgaatccaccgaaccaggaccactcaccaacaccacccgaggcgcggcaaatctcctaacaggatcccgacggaccagacgagaaatcaccctcagaacccaggccaaatgcaaccccaatctccactactggaccacacaagacgaaggcgcagccatcggattagcctggataccctatttcggaccagccgccgaaggcatttacaccgaaggcatcatgcacaaccaaaacgggctcatctgcggccttcgccagctcgccaacgaaacaactcaagccctacaactattcctccgagcaaccacagaactacgaactttttcaatcctaaaccgcaaagcaatagacttcctactacaacgttggggaggaacctgccacatcctcggtcccgactgctgcatcgaaccccacgattggaccaaaaacatcaccgacaagatagaccagattatccatgacttcatcgacaagcccctaccagaccaaaccgacaacgataactggtggactggatggcgccaatgggtccccgccggcataggcatcaccggcgtcatcatcgcagtcatcgcactcctctgcatctgtaagttcctcctctag

SEQ ID NO: 224 - RNA, 2031 nt, synthetic construct
  misc_feature 1..2031: note = SUDV GP, Gulu, Uganda 2007, optimized mRNA Sequence
  source 1..2031: mol_type = other RNA
atgggaggtctatccctccttcaactccccagagacaaattcagaaagtccagcttctttgtttgggtaatcatacttttccaaaaagcattctccatgcccctaggcgtagtcacaaactccaccctcgaagtcaccgaaatcgaccaacttgtctgcaaagatcatctcgcgtccacagaccaactaaaatcagtgggactaaacctcgaaggctccggagtctccaccgacataccctctgcaaccaaacgatggggcttccgctccggtgtacccccaaaagtagtcagctacgaagcaggcgaatgggccgaaaactgctataacctcgagataaaaaaacccgacggctccgaatgtctccccccccccccagatggagtacgcgggttcccgagatgtcgctacgttcataaagcccaggggaccggaccctgccccggcgactacgccttccacaaggacggggccttcttcctatacgaccgactcgcctccaccgtaatctaccgcggcgtaaacttcgccgaaggcgtcatcgcattccttatcctagccaaacccaaagaaaccttcctccaaagtcccccaatccgcgaagccgtaaactacaccgaaaacacctcatcctactacgccacctcctacctcgaatacgaaatcgagaatttcggcgcccaacacagcacgaccctcttcaaaatcgataataacaccttcgtacgactagaccgcccacacacaccacaatttctcttccaactcaacgacacaatccacctccaccaacaactctccaacacaacaggccgactcatatggaccctagacgcaaacatcaacgcagacataggagaatgggccttttgggagaacaaaaaaaacttatccgaacaactccggggcgaagaactctccttcgaagcactctccctcaacgaaacagaagacgacgacgccgcctcatcccggattaccaaaggtagaatatcagaccgcgccactcggaaatactccgacctcgtaccaaaaaactcaccaggtatggtacccctccacattcccgaaggtgaaacaaccctccccagccaaaattcaacagaaggcagaagagtgggagtaaacacccaggagaccataacagaaaccgctgccacgatcatcggaaccaacggtaatcatatgcaaatcagcaccatcggaatacgcccatcatcctcccaaatcccaagcagttccccaacgacagccccatcccccgaagcccaaacccccacaacccacacatcaggcccatccgtcatggctaccgaagagcccacaacaccccccggctcatcaccaggccccaccaccgaagcacccactcttacgacccccgaaaacataaccaccgccgtaaaaactgtcctcccccaagagtcaacatccaacggcctaataacatccacagtcaccggcatactcggctcactaggcctccgcaaacgctcaagaagacaaacaaacaccaaagccacaggcaaatgcaacccaaacctccactactggaccgcccaagaacaacacaacgcagccggaattgcctggataccatacttcggcccaggcgctgaaggaatatataccgaaggcctaatgcacaaccaaaatgcgctcgtctgcggcctccgccaactcgcaaacgaaaccacccaagccttacaactattcttacgcgccaccacagaactacgcacatacacgatactaaaccgaaaagcgatcgactttctcctccgccgatggggcggaacctgcagaatcctcggccccgactgctgcatcgaaccacatgactggactaaaaacatcaccgacaaaataaaccaaataatccacgacttcatcgacaatccgctccccaaccaagacaacgacgacaattggtggactggatggcgacaatggatcccagcaggcattggcatcaccggcataatcatagcaatcatcgcactactctgcgtctgcaaactactatgttaa

SEQ ID NO: 225 - RNA, 2031 nt, synthetic construct
  misc_feature 1..2031: note = TAFV GP, Cote dIvoire 1994, optimized mRNA Sequence
  source 1..2031: mol_type = other RNA
atgggcgcgtccggcatccttcaactcccccgcgaacgtttccgtaaaacctccttcttcgtatgggtaataatactcttccacaaagtcttcagcatcccactcggagttgtccataacaatacactccaagtctccgacatcgacaaatttgtttgccgagacaaactatcatcaacttcacaattaaaatccgtcggcctcaacctcgaaggtaacggcgtcgccactgacgtacccacagccaccaagcgctggggcttccgagcaggcgtcccccccaaggttgtcaattgcgaagccggcgagtgggccgaaaactgctacaacctagccatcaaaaaagtcgacggaagcgaatgcttgcccgaagctcccgaaggcgtacgtgatttcccccggtgccgatatgtccacaaagtgtccggcacaggtccctgcccaggtggcctagctttccacaaagaaggagccttcttcctctacgaccgcctcgcctccacaataatataccgcggcacaacatttgccgaaggcgtcatcgccttcctaatccttcccaaggcccgcaaagacttcttccaaagcccccctcttcacgaaccagctaatatgaccacggacccatcctcctactatcacacaacaaccatcaactacgtcgtcgacaacttcggcaccaacaccaccgaattcctctttcaagtcgaccacctcacctacgtacaactcgaggctcgcttcacaccccagttcctcgttctactaaacgaaacaatatactccgacaaccgccgctcgaacaccaccggcaaattaatatggaaaataaacccgaccgtcgacacgagcatgggagaatgggctttctgggagaacaaaaaaaacttcaccaaaacactttcatccgaagaactgtctttcgtacctgttccagaaacccaaaaccaggtactagacactaccgctacggtatccccccctatctccgcccataaccacgcagccgaagaccacaaagaactcgtctccgaagactcaaccccagtcgtacagatgcaaaacatcaaaggcaaagacaccatgccaaccacagtcaccggcgtcccaacaaccactcccagcccattcccaatcaatgcccgcaacacagaccacacaaaatccttcatcggactcgaaggaccccaagaagatcactccaccactcagccagcaaaaacaacatcacaaccaaccaactccaccgaatccactacgttaaaccccaccagcgaaccatcatccagaggaactggaccaagttccccaacagtacccaacaccaccgaatcccacgccgaactcgggaagactacccccactactctccctgaacaacacaccgccgcctcagctatcccaagagccgtccatcccgacgaactctccggacccggcttcctgaccaacacaatccgcggtgtaaccaacctcctcacaggcagcagacgtaaacgacgcgacgtaaccccaaacacccaacccaaatgcaaccctaacctacactactggaccgcattagacgaaggcgcagcgatcggcctcgcctggatcccctattttggccccgccgcagaaggcatctacaccgaaggaatcatggaaaaccagaacggtctaatctgcggcttacgccaactagcaaacgaaaccacgcaagccctccaactattcttacgtgccaccaccgagctccgaaccttttccatcctaaaccgcaaagccatcgacttccttctccaacgctggggaggcacctgccacatcctaggccccgactgctgcatcgaaccccaagactggaccaaaaacatcaccgataaaattgaccagataatccacgacttcgtcgacaacaacctccccaaccaaaacgacggatcaaactggtggaccggctggaaacaatgggtaccggcaggcatcggaatcacaggtgtaatcatagccatcatcgcactactttgcatctgcaaattcatgctttaa

SEQ ID NO: 226 - RNA, 981 nt, synthetic construct
  misc_feature 1..981: note = BDBV VP40, Uganda 2007, optimized mRNA Sequence
  source 1..981: mol_type = other RNA
atgcgccgagccatcctccccaccgccccaccagaatacatcgaagccgtctaccccatgcgcaccgtatccacctctatcaactccacagcatcaggccccaacttccccgcacccgacgtcatgatgtccgacaccccctcaaactcactccgcccaatcgccgacgacaacatagaccacccctcccacacccccacctcagtctcatccgcattcatactagaagccatggtcaacgtcatctccggccccaaagtcctaatgaaacaaattcccatctggctcccactcggcgtcgctgaccaaaagacctattccttcgactcaaccaccgcagccatcatgctagcatcatacaccatcacccacttcggcaaaacctccaacccactagtccgcatcaaccgactcggaccaggtatcccagaccaccccctccgccttctccgcatcggtaaccaagccttcctccaagaattcgtactcccacccgtccaactaccccaatacttcaccttcgacctcacagccctcaaactcatcacccaacccctaccagctgcaacctggaccgacgacacccccaccggccccaccggcatactccgccccggcatctccttccaccccaaacttcgccccatcctcttaccaggtaaaacaggcaaacgcggctcctccagcgacctaacttcccccgacaaaatccaagcaatcatgaacttcctccaagacctcaaactcgtcccgatcgaccccgctaaaaatattatgggaatcgaagtacccgaactcctcgtccaccgcctcaccggtaaaaaaatcacaaccaaaaacggccaacccataatccccatcctcctacccaaatacatcggcatggaccccatttcccaaggcgatctaaccatggtcatcacccaagattgcgacacctgccactcccccgcctccctcccccccgtctccgaaaaataa

SEQ ID NO: 227 - RNA, 981 nt, synthetic construct
  misc_feature 1..981: note = SUDV VP40, Gulu, Uganda 2000, optimized mRNA Sequence
  source 1..981: mol_type = other RNA
atgcgacgcgtcaccgtccccaccgccccccccgcctacgccgacatcggctaccccatgtccatgctcccaatcaaatcctcccgagccgtttccggtatacaacaaaaacaagaagtcctcccaggcatggacaccccttccaactcaatgcgtccagtcgccgacgacaacatcgaccacacctcacacacacccaacggcgtcgcatccgcattcatactagaagccaccgtcaacgtcatctccggccccaaagtcctaatgaaacaaatccctatctggctccccctaggcatcgccgaccaaaaaacctactccttcgactccaccaccgcagccatcatgctcgcctcctacaccatcacccacttcggcaaagccaacaacccactcgtacgcgtgaaccgcctcggccaaggcatccccgaccacccactccgactactccgcatgggcaaccaagccttcctccaagaattcgtcctcccccccgtacaactaccccaatacttcaccttcgatctcacagcactcaaactagttacccaacccctcccagccgccacatggaccgacgaaaccccctccaacctctccggagccctccgaccgggcctctcattccaccccaaactccgacccgtactcctacccggcaaaaccggaaaaaaaggccacgtctccgacctcacagcccccgacaaaatccaaaccatcgtaaacctaatgcaagacttcaaaattgtccccattgacccagcaaaatctatcatcggaatcgaagtccccgaactactcgtccacaaactcaccggcaaaaaaatgtcacaaaaaaacggccaacccatcatcccagttctactccccaaatacatcggcctcgacccaatctcccccggcgacctcaccatggtcataacccccgactacgacgactgccactcccccgcctcctgcagctacctctccgaaaaataa

SEQ ID NO: 228 - RNA, 981 nt, synthetic construct
  misc_feature 1..981: note = TAFV VP40, Cote dIvoire 1994, optimized mRNA Sequence
  source 1..981: mol_type = other RNA
atgcgccgcatcatcctacccaccgccccccccgaatacatggaagccgtctaccccatgcgaaccatgaactccggcgccgacaacaccgcatccggccctaactacaccaccacaggcgtcatgaccaacgacaccccctccaactccctccgccccgtcgccgacgacaacatcgaccacccatcccacaccccaaactctgtcgcatccgccttcatactcgaagccatggtcaatgtcatctccggacccaaagtcctcatgaaacaaatacccatctggctccccctcggcgtctccgaccaaaaaacctactccttcgactccaccacagcagccatcatgctcgccagctacaccatcacccacttcggcaaaacatccaaccccctcgtccgcataaaccgcctaggtcccggtatcccagaccaccccctacgactcctccgtatcggtaaccaagccttcctccaagaattcgtcctcccccccgtccaactcccacaatacttcacatttgacctcaccgccctaaaactaatcacacaacccctccccgccgccacctggaccgacgaaacccccgctgtcagcaccggcacactacgcccaggcatctcctttcaccctaaactccgccccatcctactccccggccgcgccggaaaaaaaggctccaactccgacctcacctcccccgacaaaatccaagccatcatgaacttcctccaagaccttaaaatcgtccctatcgaccccacaaaaaacatcatgggcatcgaagtcccagaactcctcgtccaccgcctaaccggaaaaaaaaccaccactaagaatggccaacccatcattcccatcctcctcccaaaatacatcggcctcgaccccctatcacaaggcgacctcaccatggtaattacccaagactgcgactcctgccactcacccgcctccctcccccccgtcaacgaaaaataa

SEQ ID NO: 229 - RNA, 2088 nt, synthetic construct
  misc_feature 1..2088: note = MARV NP, Angola 2005, optimized mRNA Sequence
  source 1..2088: mol_type = other RNA
atggacctacactcactccttgaactcggaacgaaaccaaccgcccctcacgtcagaaacaagaaggtcatcctcttcgacaccaaccaccaagtcagcatctgcaaccaaataatcgacgccataaactccggcatcgacctaggcgacctcctcgaaggcggcctcctaaccctctgcgtcgaacactactacaactccgacaaggacaaattcaacaccagtcccatcgcaaaatacctacgggatgccggatacgaattcgatgtgataaaaaacgcagacgccacacgattcctcgacgtgatcccgaacgagccccactattcccctcttatcctagcactcaaaaccctggagagcaccgaaagtcaaagagggcgcatcggactgttcctgtccttctgcagcctatttctaccaaaattagtagtaggagaccgcgcctctatcgaaaaagccctacgtcaggtcaccgtccatcaagaacaaggtatagtaacctaccccaaccactggctcacaacaggccacatgaaagtcatcttcggaatcctacgatcaagcttcatccttaagtttgtactcattcatcaaggagtcaatttagtcaccggacacgacgcctacgactcgatcatctcgaactccgtcggccaaacgcgcttctccggcctattaatcgttaaaaccgtcctcgaattcatcctacagaaaacagactctggagtcacactacaccccctagtacgaacctccaaagtaaaaaatgaagtagccagcttcaaacaagccctctccaacttagcccgacacggtgaatacgcccctttcgcgcgcgtactaaacctctcaggcatcaataacctcgaacacggcctctacccacaactttcagccatcgccctaggagtagcaaccgcacacggaagcacactagccggagtaaacgtaggagaacaataccagcaactccgcgaagcagcacacgacgccgaagtaaaactccaacgccgtcacgagcaccaagaaatccaagccatcgcagaagacgacgaagaacgaaaaatcctcgagcaattccacctacaaaagaccgaaatcacccactcccaaaccctggccgtcctctcccagaaacgcgaaaaactcgcacgactcgcagccgaaatcgaaaacaacatcgtcgaagaccaaggctttaaacagagccaaaaccgagtatctcagtccttcctaaacgaccccacaccagtcgaagtaacggtacaagcccgccccatcaaccgccccaccgcactccctcccccagtagactccaaaatcgaacacgaatccacagaagactcctcatcgtcctcatcgttcgttgaccttaatgaccccttcgcactactcaacgaagacgaagacacccttgacgactccgtcatgatcccatccaccacatctagagaattccaaggcataccagaacccccccgacagtcgcaagacatcgacaactcccaaggtaaacaagaagacgaaagcaccaacctcatcaaaaaaccattcctcagataccaagaactacccccagtccaagaagacgacgaatccgaatacactaccgactcccaagaatcgatagaccaacccggatcggacaatgaacaaggcgtcgacttaccacccccaccgctctacgcccaggaaaaacgccaagaccctatccagcatcccgcagtcagctcacaggaccccttcggatccatcggcgacgtcaacggcgacatcctcgagccaatacgcagccccagctccccctccgccccccaggaagacactcgcgcacgagaagcttacgagctatccccagacttcacgaactacgaagacaatcaacaaaactggccccaacgcgtcgtcacaaaaaagggtcgcaccttcctctacccaaacgaccttctccagacaaacccccccgaaagcctaatcacagccctagtcgaggaataccaaaaccccgtatccgcaaaagaactgcaggccgactggccagacatgagtttcgacgaacgccgccacgttgcaatgaacctctaa

SEQ ID NO: 230 - RNA, 2220 nt, synthetic construct
  misc_feature 1..2220: note = BDBV NP, Uganda 2007, optimized mRNA Sequence
  source 1..2220: mol_type = other RNA
atggacccacgaccaatccgcacatggatgatgcacaacacttcagaagtagaagccgactaccacaagatactcacagccggcctctcagtccaacaaggaatcgtccgccaacggatcatacctgtctaccaaatatcaaacctcgaagaagtctgccaactgataatacaagcctttgaagcgggcgtcgactttcaagacagcgcagactcatttctactcatgctttgcctccaccacgcctaccaaggggactataaacaattcctcgaatccaacgcagtcaaatacttagagggccacggctttagattcgaaatgaaaaaaaaggaaggtgtgaaaaggctagaagaactcctccccgcagcctcaagcgggaaaaacattaaacgcaccctggccgccatgcccgaagaagaaactaccgaagcaaacgcgggccaattcctatcattcgcatccctcttcctacccaaactagtcgtcggagaaaaagcgtgcctagaaaaagtccaacgccaaatacaagtacacgccgagcaaggccttatccaatacccaaccagctggcaatccgtaggccacatgatggttatcttccgactaatgcgcactaacttcctaatcaaattcctactaatacaccaaggaatgcacatggtagctgggcacgacgccaacgacgccgttatcgcaaactcagtagcccaagcccgcttctcaggcctcctgatagtcaaaaccgtactggaccacatcttacagaaaacagaacacggagtccgactacaccctctcgcacgtacagccaaagttaaaaacgaagtcagctcattcaaagcagccctagcgtccctagcccaacacggcgaatacgcaccatttgcacgcctactcaacctctcaggagtcaacaacctagaacacggactcttcccccaactctccgccatcgccctaggtgtcgcaacagcccacggaagcaccctagccggtgtcaacgtcggcgaacaataccaacagctcagagaagcagccacggaagccgaaaaacagctccaaaagtacgccgaatcccgagagctcgaccacctgggcctagacgaccaagaaaagaaaatcctcaaagacttccatcaaaagaaaaacgaaatctccttccaacaaaccaccgccatggtcacgctccgcaaggaacggctcgcaaaactaaccgaagccatcaccagcacctcaatcctaaaaaccggacgacgctacgacgatgacaacgacattcctttccccggccctataaacgacaacgagaactcaggacaaaacgacgacgacccaaccgactcgcaagacaccacgattcccgatgtaatcatcgacccaaacgacggtggctacaataattactccgactacgccaacgacgcagcatctgcccccgacgacctagtactattcgacctcgaagatgaggatgacgcagacaatccagcgcaaaacacccccgaaaaaaacgaccggcccgcaacaaccaaactccgaaacggccaagaccaagacggcaaccaaggcgaaacagcctcaccacgcgtcgctccaaaccaataccgcgacaaacccatgccccaagtacaagaccgtagcgaaaaccacgaccaaaccctacagacccaaagccgcgtccttaccccgatctccgaagaagccgacccctccgaccacaacgacggcgacaacgaatccatcccccccttagaaagcgacgacgagggttcaacagacaccaccgccgcagaaacaaaaccagccaccgcaccgccagcccccgtgtaccgatccataagcgtcgacgactcagttccaagcgaaaacatccccgcccaatccaaccaaaccaacaatgaagacaacgttcgcaacaacgctcaatccgagcaatcaatcgcagaaatgtaccagcatatactaaaaacacaaggccccttcgacgccatactctactaccacatgatgaaagaggagcccattatattctcaaccagcgacggaaaagaatacacttatcccgattcactcgaagacgaataccccccctggctctctgaaaaagaagccatgaacgaggataacagatttataaccatggatggacaacaattctactggcctgtcatgaaccaccgaaacaaattcatggcaatattgcagcaccaccgataa

SEQ ID NO: 231 - RNA, 2217 nt, synthetic construct
  misc_feature 1..2217: note = SUDV NP, Gulu, Uganda 2000,optimized mRNA Sequence
  source 1..2217: mol_type = other RNA
atggacaaacgagtacgaggctcctgggccctcggcggacaaagcgaagtcgacctagactatcacaaaatactcacagcaggcctaagcgttcaacagggcattgtccgacaacgtgtcatccccgtctacgtcgtctccgacctcgaaggaatctgccaacacataatccaagccttcgaggccggcgtcgacttccaagataacgcagactctttcctcctactcctctgccttcaccacgcctaccaaggcgaccaccgactattcctaaaatccgacgcggtccagtacctcgaaggacacggcttcagatttgaggtacgcgaaaaggaaaacgtccatcgcctagatgaactactcccgaacgtaacaggaggtaaaaacctgcgccggacccttgccgccatgcccgaggaagaaacaacagaagcaaacgcaggacaattcctttccttcgccagcctcttcctaccgaagctcgtagtaggcgaaaaagcatgcctagaaaaagtccaacgccagatacaagtgcacgccgaacaaggactaatacaataccccacctcctggcaaagcgtcggccacatgatggtgatcttcaggctaatgcgcaccaacttcctaatcaaattcctcctcatccatcagggcatgcacatggtagcaggacatgatgcaaacgacaccgtcatatcgaactccgtcgcacaggcacgattctcaggcctcctaatagtaaaaacagtactagaccacatcctgcaaaaaaccgaccttggagtccgactccaccccctagcaagaactgccaaagtcaaaaacgaagtctcctccttcaaagctgcacttggatcgctcgcaaagcacggcgaatacgccccatttgcccgcttactcaacctcagcggcgtaaacaacctagaacacggcctttacccccaactctccgccatagctctgggcgtagcaaccgcccacgggtccaccctcgccggcgttaacgtgggggaacaataccagcaactccgtgaagccgccacagaggccgaaaagcaactccaacaatacgcagaaaccagagaactagacaacctcggactagatgaacaggagaaaaagatcctcatgtcattccaccaaaaaaaaaacgaaatatcatttcaacagaccaacgcaatggtcaccctccgcaaagaacgcctagcaaaactaacggaggccatcacgacagcctccaaaataaaagtcggagaccgctaccccgacgacaacgacatcccattccccggaccaatctacgacgaaacacaccccaacccttcagacgacaacccagacgactctagagacacaaccatcccaggcggagtcgtcgacccatacgacgacgaatccaacaactaccccgattacgaagactcagcagaaggaaccacgggcgacctagaccttttcaacctcgacgacgacgacgatgactcacaacccggaccacccgaccgcggacagtccaaagaacgcgctgcacgtacccacggcctccaagaccccaccctcgacggagcaaaaaaggtcccagaactgaccccaggcagccaccaaccgggcaacctacacatcaccaagcccggcagcaacaccaaccaacctcaaggcaacatgagcagcacgctacaatccatgacccccatccaagaagaatccgaacccgacgaccagaaagacgacgacgacgaatccttgaccagcctcgactccgaaggcgatgaagacgtcgaatccgtctccggcgaaaacaacccgaccgtagcacctcccgccccagtatacaaagataccggcgtagacaccaaccaacaaaacggacccagcaacgcagtagacggccaaggaagcgagtctgaagcactcccaatcaaccctgaaaaaggctccgctctcgaagagacctactatcacctgttaaagactcaaggccctttcgaagccatcaactactaccacctaatgtccgacgagccaatcgctttctcaactgagtccggaaaggaatacatcttccccgactccctggaagaagcctaccccccctggctgtcagaaaaagaagcactcgaaaaagaaaatcgctacctcgtaatagacggccaacaatttctctggcccgtcatgtcactccaggacaagttccttgcagtcctccaacacgactga

SEQ ID NO: 232 - RNA, 2220 nt, synthetic construct
  misc_feature 1..2220: note = TAFV NP, Cote dIvoire 1994,optimized mRNA Sequence
  source 1..2220: mol_type = other RNA
atggaatcccgggcccacaaagcatggatgacccacaccgcctcggggttcgaaaccgactaccataagatcctaaccgcaggactctcggtccaacaaggcatcgtccgacaacgcgtcatacaagtacaccaagttaccaacctcgaggaaatctgccaactcatcatccaagcatttgaagcaggcgtcgatttccaggagagtgccgatagcttcctcctaatgctctgcctccaccacgcctaccaaggcgattataaacaatttctcgagtccaacgcagtcaaatacctggaaggccatggatttcgcttcgaagttcgaaaaaaggaaggcgtcaagcgcctcgaagaactcctcccggccgcatcatccggtaaaagcatccgtcgcacactagccgcaatgcccgaagaagaaaccaccgaagccaacgccggccaattcctctcattcgcaagcctattcctccccaaactcgtcgtcggagaaaaagcctgcctagagaaagtccagcgtcaaatacaggtacacagcgaacaaggcctcatacaataccccacagcctggcagagcgtcggccacatgatggtcatattcagactaatgagaacaaacttcctaatcaaattcctactcatccaccagggcatgcacatggtagccggccacgacgccaatgacgccgtcatagccaactcagtcgcccaagccagattcagcggccttctcatcgttaagaccgtactcgaccacatcctgcaaaagacagagcacggagtgcgcctacacccattagcccgaactgctaaagtcaaaaacgaagtcaacagtttcaaagccgcactcagcagcctcgcacagcacggagaatacgcacccttcgcccgcctactcaacctaagcggcgtcaacaacctagagcacggcttattcccacagctatcagccatagccctaggcgtcgcgacagcacacggctcaaccctagccggcgtcaacgtaggggaacaataccaacaactccgcgaagcagccaccgaagcagaaaaacagctccaaaaatatgctgagtcccgagaactcgaccacttaggtctagacgaccaggaaaaaaaaatccttaaagacttccatcaaaaaaagaacgaaatcagtttccagcaaacgaccgcaatggtaacactccggaaagaacgcctcgccaaactcacagaagccataacctccacatcactactcaaaaccggcaaacaatacgatgacgacaacgacatacccttccccgggccaataaacgacaacgaaaactcagagcaacaagacgacgaccccaccgactcacaagacacaaccatccccgacatcatagtcgaccccgacgacggccgatacaacaactacggcgactacccatctgaaaccgccaatgcgccagaggacctagtcctattcgacctcgaagacggcgacgaagacgaccaccggccctcatcatccagcgaaaacaacaacaaacactcactaacaggcacagactcaaacaaaacaagcaactggaaccggaatccgacaaacatgcccaaaaaagactcaacgcagaacaacgacaacccggctcaacgcgcacaagaatacgctcgagacaacatccaagacactcccaccccacatagagcactgaccccaatatcagaagaaacaggctcaaacggacacaacgaggacgacatagactccataccccccctagaatcagacgaagagaacaacacagaaacaactatcaccaccacaaaaaacacaaccgcaccccccgcaccagtatatcgcagcaactccgagaaagaacccctccctcaagaaaaatcacagaagcaacccaaccaggtctccggctcagagaataccgacaacaaaccccactcagaacaaagcgtcgaagaaatgtacagacacatcctccaaacacaaggtccattcgatgccatactctactactacatgatgacagaagaacccatcgtattttcaacatctgacggaaaagaatacgtctacccagacagcctagaaggcgaacaccccccatggctatcagaaaaagaagccctcaacgaagacaaccggttcataaccatggacgatcaacaattctactggcccgtcatgaaccaccgaaacaaatttatggcaatcctccaacaccacaagtga

SEQ ID NO: 233 - RNA, 2083 nt, synthetic construct
  misc_feature 1..2083: note = Influenza HA, optimized nucleotide sequence
  source 1..2083: mol_type = other RNA
ggggcgctgcctacggaggtggcagccatctccttctcggcatcaagcttaccatgaaggccatcctggtggtcctcctgtacaccttcgccaccgcgaacgccgacacgctgtgcatcggctaccacgccaacaacagcaccgacaccgtggacaccgtgctcgagaagaacgtcacggtgacccactccgtgaacctgctggaggacaagcacaacgggaagctctgcaagctgcggggcgtcgccccgctgcacctcgggaagtgcaacatcgccggctggatcctggggaacccggagtgcgagagcctgtccaccgcgagctcctggagctacatcgtggagacctccagctccgacaacggcacgtgctaccccggcgacttcatcgactacgaggagctccgcgagcagctgagctccgtgagctccttcgagcggttcgagatcttccccaagaccagctcctggcccaaccacgacagcaacaagggggtcaccgccgcctgcccgcacgccggcgcgaagtccttctacaagaacctgatctggctcgtgaagaaggggaacagctaccccaagctgtccaagagctacatcaacgacaagggcaaggaggtgctggtcctctgggggatccaccaccccagcacctccgccgaccagcagagcctgtaccagaacgccgacgcctacgtgttcgtgggctccagccgctactccaagaagttcaagcccgagatcgccatccggccgaaggtccgcgaccaggagggccggatgaactactactggacgctggtggagcccggggacaagatcaccttcgaggcgaccggcaacctcgtggtcccccgctacgccttcgccatggagcggaacgccgggagcggcatcatcatctccgacacccccgtgcacgactgcaacacgacctgccagaccccgaagggcgccatcaacaccagcctgcccttccagaacatccaccccatcacgatcgggaagtgccccaagtacgtgaagtccaccaagctgcgcctcgcgaccggcctgcggaacgtcccgagcatccagtcccgcgggctgttcggcgccatcgccgggttcatcgagggcggctggaccgggatggtggacggctggtacgggtaccaccaccagaacgagcagggcagcgggtacgccgccgacctcaagtccacgcagaacgcgatcgacgagatcaccaacaaggtgaacagcgtcatcgagaagatgaacacccagttcaccgccgtgggcaaggagttcaaccacctggagaagcggatcgagaacctgaacaagaaggtcgacgacggcttcctcgacatctggacgtacaacgccgagctgctggtgctcctggagaacgagcgcaccctggactaccacgactccaacgtgaagaacctctacgagaaggtccggagccagctgaagaacaacgccaaggagatcgggaacggctgcttcgagttctaccacaagtgcgacaacacctgcatggagtccgtgaagaacgggacctacgactaccccaagtacagcgaggaggccaagctgaaccgcgaggagatcgacggcgtgaagctcgagtccacgcggatctaccagatcctggcgatctacagcaccgtcgccagctccctggtgctcgtggtcagcctgggggccatctccttctggatgtgcagcaacggctccctgcagtgccgcatctgcatctgaccactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt